SEQUENCE LISTING (2 sequences)

SEQ ID NO: 1 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 2 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

(19) United States
(12) Patent Application Publication
(10) Pub. No.: US 2014/0329863 A1
(43) Pub. Date: Nov. 6, 2014

(54) NOVEL ANTIPRION COMPOUNDS

(71) Applicant: The Regents of the University of California, Oakland, CA (US)

(72) Inventors: Adam R. Renslo, Oakland, CA (US); Alejandra Gallardo-Godoy, San Francisco, CA (US); Michael B. Silber, San Francisco, CA (US); Stanley B. Prusiner, San Francisco, CA (US); Kurt Giles, San Francisco, CA (US); Zhe Li, San Francisco, CA (US); R. Jeffrey Neitz, San Francisco, CA (US)

(73) Assignee: The Regents of the University of California, Oakland, CA (US)

(21) Appl. No.: 14/191,008

(22) Filed: Feb. 26, 2014

Related U.S. Application Data
(63) Continuation of application No. PCT/US2012/052558, filed on Aug. 27, 2012.
(60) Provisional application No. 61/528,131, filed on Aug. 26, 2011.
(60) Provisional application No. 61/639,853, filed on Apr. 27, 2012.
(60) Provisional application No. 61/639,858, filed on Apr. 27, 2012.

(51) Int. Cl. C07D 417/14; C07D 417/12

(52) U.S. Cl. CPC C07D 417/14; C07D 417/12
USPC 514/333; 546/256; 546/270.7; 514/342

(57) ABSTRACT

Described herein are novel compositions and methods of treatment addressing diseases such as neurodegenerative diseases, including prion diseases and Alzheimer's disease.

### CROSS-REFERENCES TO RELATED APPLICATIONS

This application claims the benefit of U.S. Provisional Patent Application No. 61/528,131, filed Aug. 26, 2011, 61/639,853, filed Apr. 27, 2012, and 61/639,858, filed on Apr. 27, 2012, which are all incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with government support under grants AG021601, AG031220, AG002132, and AG10770 awarded by the National Institute of Health. The Government has certain rights in the invention.

### REFERENCE TO A “SEQUENCE LISTING,” A TABLE, OR A COMPUTER PROGRAM LISTING APPENDIX SUBMITTED ON A COMPACT DISK

The Sequence Listing written in file 84850-847781_ST25.TXT, created on Aug. 24, 2012, 4,971 bytes, machine format IBM-PC, MS-Windows operating system, is hereby incorporated by reference.

### BACKGROUND OF THE INVENTION

The protein misfolding diseases are a family of debilitating neurological disorders associated with the misprocessing of cellular proteins into alternate non-native isoforms that confer cellular toxicity, often associated with oligomeric deposits derived from misfolded protein. Prominent examples of these disorders include Alzheimer's disease, Huntington's disease, Parkinson's disease, frontotemporal dementias, and the prion diseases Creutzfeldt-Jakob disease in humans, chronic wasting disease in deer, and scrapie in sheep.^{2,3} In prion disease, the endogenous prion protein (PrP^{C}) is converted by an unknown mechanism into a protease-resistant and β-sheet-rich form denoted PrP^{Sc}. This conversion can occur spontaneously, result from inherited mutations in the PrP^{C} gene, or be triggered by infection with exogenous PrP^{Sc}. Prion diseases are invariably fatal, and no viable treatments for these devastating disorders are currently available.

Prion diseases belong to a class of neurodegenerative disorders characterized by the aberrant processing and aggregation of a normally innocuous soluble host protein, denoted the cellular prion protein (PrPC).^{1,2} Disease onset is associated with the accumulation of a β-sheet-rich, infectious isoform, termed PrP^{Sc}, which is formed from the α-helixrich PrP^{C}.^{3, 4}

Human prion diseases include Creutzfeldt-Jakob disease (CJD), kuru, and Gerstmann-Straüssler-Scheinker syndrome.⁵ CJD shares histopathological findings of aggregated misfolded protein deposits in the brain with other human neurodegenerative diseases and proteinopathies, including Alzheimer's disease (AD); Parkinson's disease (PD); tauopathies, such as frontotemporal dementia (FTD); Huntington's disease (HD); and amyotrophic lateral sclerosis (ALS).^{4, 8} AD, HD, PD, and tauopathies involve misprocessing of specific, cellular proteins into alternate non-native isoforms that produce cellular toxicity; these pathogenic proteins then propagate in a prion-like process.^{9, 10, 8, 11-13}

While the mechanisms of protein misfolding and subsequent disease progression remain unclear, it is well known that infectious forms of animal prions can be propagated in cell culture, notably in prion-infected, murine neuroblastoma (ScN2a) cell lines.^{4, 5} Various immunological methods are available to measure prion load in these cell lines and so they have provided a valuable means for evaluating the antiprion properties of large and small molecules alike. Among small molecules that have been reported to possess antiprion properties are the acridines^{6, 7} (e.g., quinacrine, 1 in FIG. 1) and structurally related tricyclic antidepressants; dimeric⁸ and chimeric⁹ analogs of 1; statins¹⁰; 2,4-diphenylthiazole and 2,4-diphenyloxazole amides¹¹; pyrazolones^{12, 13}; indole-3-glyoxylamides¹⁴; and pyridyl hydrazones¹⁵ (e.g., “compound B”, 2). In addition, larger molecules of a polyanionic chemotype (suramin, pentosan polysulfate) or polycationic chemotype (dendritic polyamines, cationic polysaccharides), or PAMAM have been reported to exhibit antiprion activity in cells.^{35, 36} In fact, no small molecule has yet been shown to be broadly effective against a range of prion strains in an animal model of disease¹⁷ and only hydrazone 2 in FIG. 21 has been reported to significantly extend survival in animals (albeit strain-dependent and at high doses).¹⁵

In both cells and animal models, transmission and propagation of prions can be titrated by controlling levels of PrP^{C} expression and prevented if PrP^{C} expression is abolished by knockout (KO) of the PrP gene.¹⁴⁻¹⁷

Because PrP-null mice have no apparent deficits or developmental problems, and have a normal life span,¹⁸ a therapy that effectively lowers PrP^{C} levels might be therapeutically efficacious and well tolerated. In contrast to PrP^{C}, PrP^{Sc} forms insoluble fibrils, then aggregates, some form of which is neurotoxic.¹⁹⁻²¹ Therapies targeting PrP^{Sc} in the brain by halting the formation or increasing clearance should also be therapeutically desirable.

### BRIEF SUMMARY OF THE INVENTION

In a first aspect is a compound having the formula:

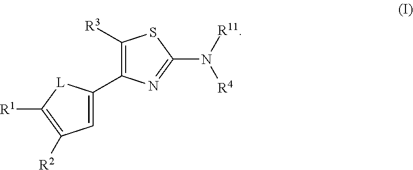

L is —CR⁶═CH—, —S—, or —O—. R⁷, R⁸, R⁹ and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Where each R⁷, R⁸, R⁹, and R¹⁰ is different, they may be referred to, for example, as R⁷′, R⁷″, R⁷′″, R⁷″″, and so on. R¹¹ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —C(O)R¹², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹² is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl substituted or unsubstituted heterocycloalkyl, substituted or unsubstituted aryl, or substituted or unsubstituted heteroaryl. R¹, R², R³, R⁴, and R⁶ are independently hydrogen, halogen, —CX₃, —OCX₃, —CN, —SO₂Cl, —SOR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryThe symbol v is independently 1 or 2. The symbol m is independently an integer from 1 to 2. The symbol n is independently an integer from 0 to 4. X is independently Cl, —Br, —I, or —F.

Two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl. Adjacent R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl. The symbol t4 is an integer from 0 to 2. Adjacent R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl. Adjacent R¹ and R² substituents may optionally be joined to form a substituted or unsubstituted cycloalkyl, substituted or unsubstituted heterocycloalkyl, substituted or unsubstituted aryl, or substituted or unsubstituted heteroaryl.

In another aspect is a compound having the formula:

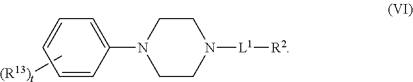

X, m, n, v, R², R⁷, R⁸, R⁹, and R¹⁹ are as described herein (e.g. formula (I) to (V), including embodiments). L¹ is a bond, substituted or unsubstituted alkylene, substituted or unsubstituted heteroalkylene, —NR^{1A}—, —O—, —S—, —C(O)—, or —CHR^{1A}—. R^{1A} is independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹³ is independently hydrogen, halogen, —CX^{a}₃, —CN, —SO₂Cl, —SOᵣR¹⁷, —SOₚNR¹⁴R¹⁵, NHNH₂, ONR¹⁴R¹⁵, —NHC═(O)NHNH₂, —NHC═(O)NR¹⁴R¹⁵, —N(O)_{q}—, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl.

R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol t is independently an integer from 0 to 5. The symbol q is independently an integer from 1 to 2. The symbol r is independently an integer from 0 to 4. X^{a} is independently —Cl, —Br, —I, or —F. Y is independently, —N═, or —N⁺(O⁻)═, or —C(R¹³)═. Where each R¹⁴, R¹⁵, R¹⁶, and R¹⁷ is different, they may be referred to, for example, as R¹⁷′, R¹⁷″, R¹⁷′″, R¹⁷″″, and so on.

R^{13a}, R^{13b}, and R^{13c} are independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{13a} and R^{13b} or R^{13b} and R^{13c} may optionally be joined to form a substituted or unsubstituted cycloalkyl, substituted or unsubstituted heterocycloalkyl, substituted or unsubstituted aryl, or substituted or unsubstituted heteroaryl.

In another aspect is a compound having the formula:

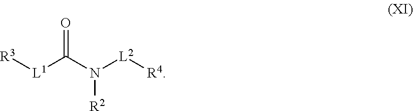

L¹, R^{1A}, X, m, n, v, R², R³, R⁴, R⁷, R⁸, R⁹, and R¹⁰ are as described herein (e.g. formula (I) to (X), including embodiments). L² is a bond, substituted or unsubstituted alkylene, substituted or unsubstituted heteroalkylene, —NR^{1B}—, —O—, —S—, —C(O)—, or —CHR^{1B}—.

R^{1B} is independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O), —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In another aspect, is a compound having the formula:

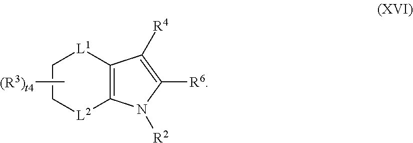

L¹, L², x, R^{1A}, R^{1B}, R², R³, R⁴, R⁶, R⁷, R⁸, R⁹, R¹⁰, m, n, and v are as described herein (e.g. formula (I) to (XV), including embodiments). The symbol t4 is an integer from 0 to 2.

In another aspect is a compound having the formula:

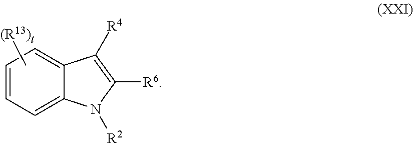

R², R⁴, R⁶, R⁷, R⁸, R⁹, R¹⁰, R¹³, R¹⁴, R¹⁵, R¹⁶, R¹⁷, X, X^{a}, m, v, n, p, q, r, and t are as described herein (e.g. formula (I) to (XX), including embodiments).

In another aspect is a compound having the formula:

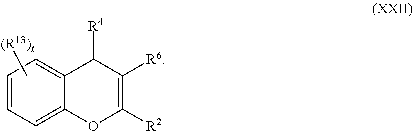

R², R⁴, R⁶, R⁷, R⁸, R⁹, R¹⁰, R¹³, R¹⁴, R¹⁵, R¹⁶, R¹⁷, X^{a}, m, v, n, p, q, r, and t are as described herein (e.g. formula (I) to (XXI), including embodiments).

In another aspect is a compound having the formula:

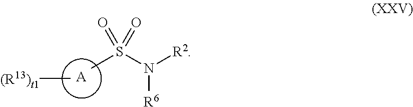

Ring A, R², R⁶, R⁷, R⁸, R⁹, R¹⁰, R¹³, R¹⁴, R¹⁵, R¹⁶, R¹⁷, X, X^{a}, m, p, v, n, r and q are as described herein (e.g. formula (I) to (XXIV), including embodiments).

The symbol t1 is independently an integer from 0 to 8.

Ring A is substituted or unsubstituted cycloalkyl, substituted or unsubstituted heterocycloalkyl, substituted or unsubstituted aryl, or substituted or unsubstituted heteroaryl.

In another aspect is a compound having the formula:

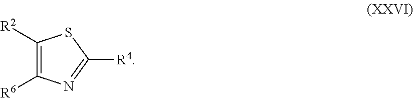

R², R⁴, R⁶, R⁷, R⁸, R⁹, R¹⁰, X, m, v, and n are as described herein (e.g. formula (I) to (XXV), including embodiments).

In another aspect is a compound having the formula:

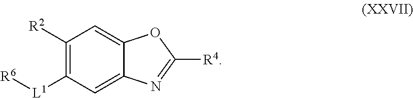

L¹, R^{1A}, R², R⁴, R⁶, R⁷, R⁸, R⁹, R¹⁰, X, m, v, and n are as described herein (e.g. formula (I) to (XXVI), including embodiments).

In another aspect is a compound having the formula:

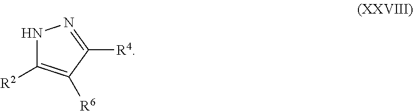

R², R⁴, R⁶, R⁷, R⁸, R⁹, R¹⁰, X, m, v, and n are as described herein (e.g. formula (I) to (XXVII), including embodiments).

In another aspect is a compound having the formula:

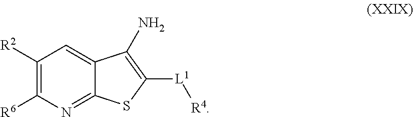

L¹, R^{1A}, R², R⁴, R⁶, R⁷, R⁸, R⁹, R¹⁰, X, m, v and n are as described herein (e.g. formula (I) to (XXVIII), including embodiments).

In another aspect is a compound having the formula:

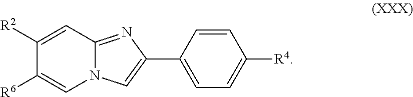

R², R⁴, R⁶, R⁷, R⁸, R⁹, R¹⁰, X, m, v, and n are as described herein (e.g. formula (I) to (XXIX), including embodiments).

In another aspect is a compound having the formula:

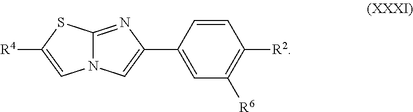

R², R⁴, R⁶, R⁷, R⁸, R⁹, R¹⁰, X, m, v, and n are as described herein (e.g. formula (I) to (XXX), including embodiments).

In another aspect is a compound having the formula:

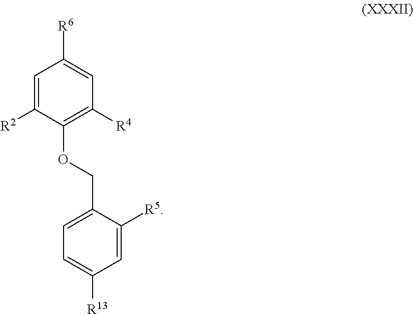

X, X^{a}, m, n, p, q, r, v, R², R⁴, R⁶, R⁷, R⁸, R⁹, R¹⁰, R¹³, R¹⁴, R¹⁵, R¹⁶, and R¹⁷ are as described herein (e.g. formula (I) to (XXXI), including embodiments).

R⁵ is independently hydrogen, halogen, —CX^{b}3, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl.

R^{7b}, R^{8b}, R^{9b}, and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Where each R^{7b}, R^{8b}, R^{9b}, and R^{10b} is different, they may be referred to, for example, as R^{7b}′, R^{7b}″, R^{7b}′″, R^{7b}″″, and so on. The symbol v1 is independently 1 or 2. The symbol m1 is independently an integer from 1 to 2. The symbol n1 is independently an integer from 0 to 4. X^{b} is independently —Cl, —Br, —I, or —F.

In another aspect is a compound having the formula:

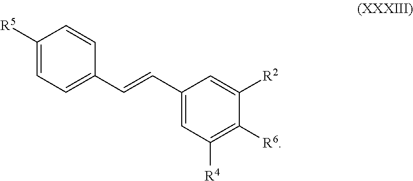

X, X^{b}, m, n, v, m, vl, nl, R², R⁴, R⁵, R⁶, R⁷, R⁸, R⁹, R¹⁰, R^{7b}, R^{8b}, R^{9b}, and R^{10b} are as described herein (e.g. formula (I) to (XXXII), including embodiments).

In another aspect is a compound having the formula:

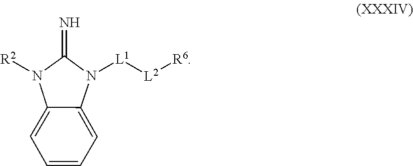

L¹, L², R^{1A}, R^{1B}, X, m, n, v, R², R⁶, R⁷, R⁸, R⁹, and R¹⁰ are as described herein (e.g. formula (I) to (XXXIII), including embodiments).

In another aspect is a compound having the formula:

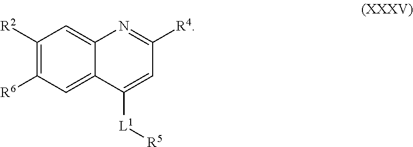

L¹, R^{1A}, X, X^{b}, m, n, v, m1, v1, n1, R², R⁴, R⁵, R⁶, R⁷, R⁸, R⁹, R¹⁰, R^{7b}, R^{8b}, R^{9b}, and R^{10b} are as described herein (e.g. formula (I) to (XXXIV), including embodiments).

In another aspect is a compound having the formula:

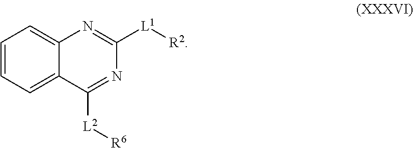

L¹, L², R^{1A}, R^{1B}, X, m, n, v, R², R⁶, R⁷, R⁸, R⁹, and R¹⁰ are as described herein (e.g. formula (I) to (XXXV), including embodiments).

In another aspect is a compound having the formula:

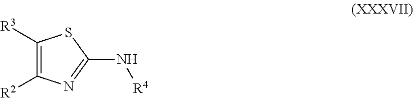

X, m, n, v, R², R³, R⁴, R⁷, R⁸, R⁹, and R¹⁰ are as described herein (e.g. formula (I) to (XXXVI), including embodiments).

In another aspect is a compound having the formula:

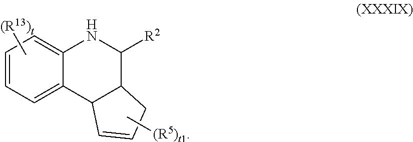

X, X^{a}, X^{b}, m, n, p, q, r, v, m1, v1, n1, t, t1, R², R⁵, R⁶, R⁷, R⁸, R⁹, R¹⁰, R^{7b}, R^{8b}, R^{9b}, R^{10b}, R¹³, R¹⁴, R¹⁵, R¹⁶, and R¹⁷ are as described herein (e.g. formula (I) to (XXXVIII), including embodiments).

In another aspect is a compound selected from any of the tables, figures, or charts provided herein (e.g. table 2, 4, 5, 6, 8, 9, 18, 19, 20, 24, 25, 26, 27, 28, FIG. 20, 31, Chart 1, 2, 3, 4, or 5).

In another aspect is a pharmaceutical composition including a pharmaceutically acceptable excipient and a compound as provided herein (e.g. formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), or (XLII), including embodiments; a compound selected from any of the tables, figures, or charts provided herein (e.g. table 2, 4, 5, 6, 8, 9, 18, 19, 20, 24, 25, 26, 27, 28, FIG. 20, 31, Chart 1, 2, 3, 4, or 5).

In another aspect is a method of treating a disease in a patient in need of such treatment, the method including administering a therapeutically effective amount of a compound as provided herein (e.g. formula (I), (II), (III), (IV), (V), (VI), (VII), (VII), (VII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), or (XLII), including embodiments; a compound selected from any of the tables, figures, or charts provided herein (e.g. table 2, 4, 5, 6, 8, 9, 18, 19, 20, 24, 25, 26, 27, 28, FIG. 20, 31, Chart 1, 2, 3, 4, or 5).

In another aspect is a method of decreasing the amount of a prion protein in a cell, the method including contacting the cell with a compound as provided herein (e.g. formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), or (XLII), including embodiments; a compound selected from any of the tables, figures, or charts provided herein (e.g. table 2, 4, 5, 6, 8, 9, 18, 19, 20, 24, 25, 26, 27, 28, FIG. 20, 31, Chart 1, 2, 3, 4, or 5).

### BRIEF DESCRIPTION OF THE DRAWINGS

Chart 1. Chemical structures and antiprion activity of select AMT compounds.

Chart 2. Chemical structures and antiprion activity of select AMT compounds.

Chart 3. Chemical structures and antiprion activity of select AMT compounds.

Chart 4. Chemical structures and antiprion activity of analogs 38 and 39, in which ring fusion enforces a co-planar A/B-ring conformation. Activities of the corresponding unconstrained analogs 25 and 14 are shown for comparison.

Chart 5. Chemical structures and antiprion activity of analogs with modified A-B ring linkages (46) or B-C ring linkages (47-50). The introduction of an amide linkage was better tolerated at the A-B ring connection (46) than at the B-C ring connection (47 and 48). Alkylation or acylation of the amino B-C ring linkage was tolerated (17 vs 49 and 50) demonstrating that a hydrogen bond donor may not be required in this position.

Table 1 to 34.

FIG. 1. Z-scores in 190 assay runs for PrP^{C} in human IMR32 neuroblastoma (open circles) and T98G glioblastoma (filled circles) cells.

FIG. 2. Distribution of inhibition of PrP^{C} in IMR32 cells, by chemical scaffold (top). The number of compounds tested for each chemical scaffold is indicated (bottom).

FIG. 3. Distribution of inhibition of PrP^{C} in T98G cells, by chemical scaffold (top). The number of compounds tested for each chemical scaffold is indicated (bottom).

FIG. 4. Z′ and or Z scores for 200 assay runs in dividing (open circles) and stationary (filled circles) ScN2a-c13 cells, respectively.

FIG. 5. Distribution of inhibition of PrPsc in dividing ScN2a-c13 cells, by chemical scaffold (top). Inhibition of PrPsC is expressed as a relative percentage compared to PrP^{Sc} in ScN2a-c13 cells treated with DMSO (negative control). The number of compounds tested for each chemical scaffold is indicated (bottom).

FIG. 6. Distribution of inhibition of PrP^{Sc} in nondividing (stationary) ScN2a-c13 cells, by chemical scaffold (top). Inhibition of PrP^{Sc} is expressed as a relative percentage compared to PrP^{Sc} in ScN2a-c13 cells treated with DMSO (negative control). The number of compounds tested for each chemical scaffold is indicated (bottom).

FIG. 7. (left) EC₅₀ and LC₅₀ curves for three IND compounds from the aminothiazole (a), benzamide (b), and benzoxazole (c) lead series in dividing ScN2a-c13 cells. EC₅₀ values are from dose-response experiments in ELISA and Western immunoblots (right); LC₅₀ values are from calcein AM assays. Percent maximal inhibition is normalized to the maximal absorbance measured at the highest concentration tested. In the Western blots, actin levels are shown as a control. Molecular masses are based on the migration of protein standards, shown in kilodaltons (kDa).

FIG. 8. Comparison of EC₅₀s (nM) from ELISAs and Westerns (dividing ScN2a-c13 cells) (N=3). Dashed line represents the line of identity, solid black line represents the least-squares regression fit. Concentrations are plotted log-log

FIG. 9. EC₅₀ results in dividing ScN2a-c13 cells by scaffold, expressed as a percentage of total compounds tested for each scaffold. For each scaffold, potency (<1 μM in middle row, 1-10 μM in last row from front) and coplanarity (front row) are shown. For the coplanarity row, a column with an intercept of 0.1 indicates coplanar compounds and a column with an intercept of ˜0 indicates noncoplanar compounds.

FIG. 10. Number of compounds that lower PrP^{C} levels in IMR32 cells and T98G cells, and also reduce PrP^{Sc} levels in dividing ScN2a-c13 cells (dPrP^{Sc}) and nondividing ScN2a-c13 (ndPrP^{Sc}). All possible combinations for the 4 assays are shown, with the exact number of compounds indicated above each bar.

FIG. 11. Brain and plasma concentration in mice after oral administration of 10 mg/kg of IND-0052851 (a) and IND-0045193 (b). Chemical structures for these are found in Table 2. Data points and bars signify the mean±SD representing two mice at each time point. Each drug was given in a separate experiment.

FIG. 12. Metabolic profile (UV chromatogram at 268 nm) IND24 in human, mouse, rat and dog liver microsomal incubations (A). The parent drug is not shown in the chromatogram. Metabolites M2, M3 and M4 had the same mass spectrum as depicted in Table 7. Extracted ion chromatogram and mass spectrum of unchanged IND24 following liver microsomal incubations (B).

FIG. 13. Extracted ion chromatograms of oxidative metabolites of IND81 at m/z 367 following its incubation with human, mouse, rat and dog liver microsomes (A). The UV chromatogram of the metabolic profile could not be obtained due to low intensity of the metabolites in the incubation mixture. Extracted ion chromatogram and mass spectrum of IND81 following incubation with liver microsomes (B).

FIG. 14. Metabolic schemes of IND24 (A) and IND81 (B) following incubation with human, mouse, rat and dog liver microsomes. For IND24, metabolite M4 was only observed following incubation with dog liver microsomes.

FIG. 15. Brain exposure (AUCₗₐₛₜ [μM*h]) to 2-AMTs after a single 40 mg/kg (A) or 10 mg/kg (B) oral dose.

FIG. 16. The ratios of maximal brain concentration (Cₘₐₓ) to EC₅₀ value for 2-AMTs following a single 40 mg/kg (A) or 10 mg/kg (B) oral dose.

FIG. 17. “Steady-state” concentrations (Cₛₛ) of IND24 (A), IND81 (B), and IND54304 (C) in brain (▴) and plasma (▪) following three-day dosing in liquid diet. For IND24 and IND81, doses were 40, 80, 130 and 210 mg/kg/day. For IND54304, doses were 25, 50, 100 and 210 mg/kg/day.

FIG. 18. “Steady-state” concentrations (Cₛₛ) of 10 2-AMT compounds in brain and plasma following three-day dosing in liquid diet. Doses were 40, 80, 130 and 210 mg/kg/day. Mean values (N=2) are plotted; mean±SD values are shown in Table 22. In a couple of cases, concentrations at low doses were below the lower level of quantification by LC/MS/MS; these brain concentrations appear as missing values.

FIG. 19. Brain (black bars) and plasma (white bars) concentrations of IND24 when dosed orally for 3 days at 75 mg/kg in female FVB mice in formulations containing varying final amounts of PEG 400. Mean±SD values are shown.

FIG. 20. Examples of compounds with antiprion activity.

FIG. 21. Structures of small molecules with antiprion properties.

FIG. 22. Summary of structure-antiprion activity relationships for 2-aminothiazole analogs. The three rings are arbitrarily denoted A, B, and C for convenience.

FIG. 23. Brain and plasma concentrations (μM) of aminothiazole 27 in mice after three days of feeding. Compound 27 was administered at the indicated doses as part of a rodent liquid diet (n=3 per dosing group). Shows that 27 is brain penetrant.

FIG. 24. 1D NOESY Spectra for compound 49, supporting the assigned sites of methylation and acylation respectively. Observed NOEs are between the CH₃ group (N—CH₃ or N—Ac) and the indicated positions on the quinoline ring.

FIG. 25. 1D NOESY Spectra for compound 50, supporting the assigned sites of methylation and acylation respectively. Observed NOEs are between the CH₃ group (N—CH₃ or N—Ac) and the indicated positions on the quinoline ring.

FIG. 26: Distribution of inhibition of PrP^{C} for confirmed SPC hits (N=138) in T98G cells, by chemical scaffold (top). The number of compounds tested for each chemical scaffold is indicated (bottom).

FIG. 27: Distribution of inhibition of PrP^{C} for confirmed SPC hits (N=114) in IMR32 cells, by chemical scaffold (top). The number of compounds tested for each chemical scaffold is indicated (bottom).

FIG. 28: EC₅₀ ELISA and calcein (cell viability) curves for two potent confirmed SPC hits in T98G cells from each of three representative scaffolds: Amide (A and B), Aminothiazole (C and D), and Chromene (E and F). Percent maximal inhibition is normalized to the maximal absorbance measured at the highest concentration tested. See Table 2 for exact EC₅₀ values.

FIG. 29: EC₅₀ ELISA (Y-axis on left) and calcein (cell viability) (Y-axis on right) curves for three potent confirmed SPC hits in T98G (A, C, E) and N2a-c13 (B, D, F) cells. Percent maximal inhibition is normalized to the maximal absorbance measured at the highest concentration tested. See Table 2 for exact EC₅₀ values.

FIG. 30: Confirmed SPC hit overlapping in the PrPC assays in T98G and IMR32 cells and in the ScN2a-c13 assay in dividing cells.

FIG. 31: Concentrations in brain (▪) and plasma () after a single 10 mg/kg PO dose, and in brain (♦) and plasma (▴) after a single 10 mg/kg IP dose for six confirmed hits, each representing a unique chemical scaffold. IND8541 (A), IND30802 (B), IND87406 (C), IND116071 (D), IND116088 (E), IND126328 (F).

FIG. 32: Cₘₐₓ(brain):EC50 ratio after a single 10 mg/kg PO (left) and IP (right) dose for 28 confirmed SPC hits tested in potency (EC50) and in vivo pharmacokinetic studies. Brain concentrations were below the lower level of quantitation after PO dosing for 14 of 28, precluding calculation of a ratio for these.

FIG. 33: Brain (A) and plasma (B) concentrations of IND30802 and brain (C) and plasma (D) concentrations of IND84706 after three days of IP dosing at 20 (square), 50 (circle), and 100 (triangle) mg/kg QD.

FIG. 34: Metabolic stability of 6 confirmed hits, representing six chemical scaffolds, in mouse () and human (▪) liver microsomes. IND-0008541 (A), IND-0030802 (B), IND-0087406 (C), IND-0116071 (D), IND-0116088 (E), IND-0126328 (F).

FIG. 35: Potency (EC50) curves as measured by ELISA (solid circle) for IND24 (1.27 μM), IND81 (1.95 μM), IND22 (1.46 μM), IND120 (0.229 μM), IND112 (0.251 μM), and IND52 (4.95 μM), and by calcein for cell viability (open circle), where EC50 for all six was >10 μM. All EC50 values are based on N=3.

FIG. 36: AMT analogs synthesized and tested for potency with calculated parameters.

FIG. 37: Metabolic stability and hepatic extraction ratio in animal (by gender) and human microsomes.

FIG. 38: Single-dose plasma pharmacokinetics of IND24, IND81, IND22 and Compd B in female FVB mice. Intravenous (IV) dose was 1 mg/kg; dose by oral gavage (PO) was 10 mg/kg. Mean±SD shown for Cₘₐₓ and AUC values (N=2).

FIG. 39: Synthesis of compound B.

FIG. 40: (FIG. 5A) Metabolic profile (UV chromatogram at 268 nm) of IND24 in human, mouse, rat and dog liver microsomal incubations. The parent drug is not shown in the chromatogram. (FIG. 5B) Extracted ion chromatogram and mass spectrum of unchanged IND24 following liver microsomal incubations.

FIG. 41: Brain exposure (AUClast [μM*h]) to 2-AMTs and Compd B after a single 40 mg/kg (A) or 10 mg/kg (B) oral dose.

FIG. 42: The ratios of maximal brain concentration (Cmax) to EC50 value for 2-AMTs and Compd B following a single 40 mg/kg (A) or 10 mg/kg (B) oral dose.

FIG. 43: “Steady-state” concentrations (Css) of IND24 (A), IND81 (B), IND22 (C) and Compd B (D), in brain (▴) and plasma (▪) following three-day dosing in liquid diet. For IND24, IND81, and IND22 doses were 25, 75, 125 and 210 mg/kg/day. For Compd B, doses were 25, 50, 100 and 150 mg/kg/day.

FIG. 44: Examples of leads that lower PrP^{Sc} levels in dividing and nondividing ScN2a-c13 cells.

FIG. 45: z-scores in 190 assay runs for PrPc in human IMR32 meuroblastoma (O) and T98G glioblastoma () cells.

FIG. 46. AUC values for brain (black) and plasma (white) and AUC/EC50 ratios for brain (vertical stripes) and plasma (horizontal stripes) based on brain and plasma concentrations for ten AMT analogs and Compd B. Values are based on a dose of 210 or 100 mg/kg/day given for 3 days for the AMT analogs and Compd B, respectively. AUC values calculated from C_{3-day}×τ, as described in the Methods.

FIG. 47: AUC values for brain (black) and plasma (white) and AUC/EC₅₀ ratios for brain (vertical stripes) and plasma (horizontal stripes) based on brain and plasma concentrations and corrected for fraction unbound for IND81, IND24, and Compd B. Values are based on a dose of 210 mg/kg/day (IND81 and IND24) or 100 mg/kg/day (Compd B) given for 3 days. AUC values calculated from C_{3-day}×τ, as described in the Methods.

FIG. 48: Compounds. “EC50” determined with ScN2a-c13 assay, as described herein. “EC50 (T98G)” determined with PrPC assay as described herein. Cmax values refer to maximal brain concentrations following dosing to mice. PO refers to oral dosing, IP to intraperitoneal dosing. C24 hr values refer to brain concentration in mice 24 hrs after a three-day dosing regimen

FIG. 49: Compounds“EC50” determined with ScN2a-c13 assay, as described herein.

FIG. 50: Compounds “EC50” determined with ScN2a-c13 assay, as described herein.

FIG. 51: Compounds

FIG. 52: Compounds “EC50” determined with ScN2a-c13 assay, as described herein. “EC50 (T98G)” determined with PrPC assay as described herein. Cmax values refer to maximal brain concentrations following dosing to mice. PO refers to oral dosing, IP to intraperitoneal dosing.

FIG. 53: Compounds “EC50” determined with ScN2a-c13 assay, as described herein. “EC50 (T98G)” determined with PrPC assay as described herein.

Scheme 1. General procedure for synthesis of antiprion 2-aminothiazoles. Conditions: (a) PhSCN, acetone, reflux; (b) NaOH, MeOH, reflux; (c) EtOH, reflux.

Scheme 2. ^{α}Hantzsch-type synthesis of 2-aminothiazole analogs from amines via thiourea intermediates. Reagents and conditions: (a) PhSCN, acetone, reflux; (b) NaOH, MeOH, reflux; (c) bromoacetophenone, EtOH, reflux.

Scheme 3. Synthesis of aminothiazole analogs 38 and 39 bearing a fused A-B ring system. Reagents and conditions: a) Br₂, Et₂O (for 65); b) 52, EtOH, 60° C.

Scheme 4. Synthesis of aminothiazole analog 46. Reagents and conditions: a) Ethyl bromopyruvate, EtOH; b) 5N HCl, MW, 10 min, 130° C.; c) 3,4-dimethoxyphenylamine, HATU, THF.

Scheme 5. Synthesis of aminothiazole analog 48. Reagents and conditions: a) NH₂C(═S)CO₂Et, EtOH; b) 5N NaOH, MeOH; c) isoquinolin-3-ylamine, HATU, THF

Scheme 6. Synthesis of aminothiazole analog 47. Reagents and conditions: a) NH₂C(═S)NH₂, EtOH; (ii) 5N NaOH, MeOH; b) isoquinoline-3-carboxylic acid, HATU, Et₃N, THF.

Scheme 7. Synthesis of aminothiazole analogs 49-50. Reagents and conditions: a) NaH, MeI, THF (for 49); b) acetic anhydride, 100° C. (for 50)

Scheme 8. Synthesis of Compd B.

Seqence Listing. (A) P04156, amino acid sequence of human prion protein, PrP; (B) P04925, amino acid sequence of mouse prion protein, PrP.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′-C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2 m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′-C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

A heteroaryl group substituent may be a —O⁻ bonded to a ring heteroatom nitrogen.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A)-OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₉ cycloalkyl, and each substituted or unsubstituted heterocycloalkyl is a substituted or unsubstituted 4 to 9 membered heterocycloalkyl, and each substituted or unsubstituted aryl is a substituted or unsubstituted 5 to 14 membered aryl, and each substituted or unsubstituted heteroaryl is a substituted or unsubstituted 5 to 14 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl, and each substituted or unsubstituted aryl is a substituted or unsubstituted 5 to 10 membered aryl, and each substituted or unsubstituted heteroaryl is a substituted or unsubstituted 5 to 10 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₉ cycloalkyl, and/or each substituted or unsubstituted heterocycloalkyl is a substituted or unsubstituted 3 to 9 membered heterocycloalkyl, and/or each substituted or unsubstituted aryl is a substituted or unsubstituted 5 to 14 membered aryl, and/or each substituted or unsubstituted heteroaryl is a substituted or unsubstituted 5 to 14 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₉ cycloalkylene, each substituted or unsubstituted heterocycloalkylene is a substituted or unsubstituted 3 to 9 membered heterocycloalkylene, and/or each substituted or unsubstituted arylene is a substituted or unsubstituted 5 to 14 membered arylene, and/or each substituted or unsubstituted heteroarylene is a substituted or unsubstituted 5 to 14 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl, and/or each substituted or unsubstituted aryl is a substituted or unsubstituted 5 to 10 membered aryl, and/or each substituted or unsubstituted heteroaryl is a substituted or unsubstituted 5 to 10 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene, and/or each substituted or unsubstituted arylene is a substituted or unsubstituted 5 to 10 membered arylene, and/or each substituted or unsubstituted heteroarylene is a substituted or unsubstituted 5 to 10 membered heteroarylene. In some embodiments, the compound is a chemical species set forth in the Examples section below or in any of the tables or figures or charts included herein.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853). The methods above may be used to synthesize single molecular species.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. For example certain methods herein treat prion disease by decreasing the production of PrP^{C} or PrP^{Sc}, decreasing the formation of PrP^{Sc} from PrP^{C}, decreasing the existing amount of PrP^{Sc}, decreasing the amount of PrP^{Sc} oligomers, decreasing the amount of PrP^{Sc} aggregates, increasing the clearance of PrP^{Sc}, increasing the degradation of PrP^{Sc}, increasing survival, increasing mental wellbeing, increasing mental function, slowing the decrease of mental function, or extending survival. For example certain methods herein treat Alzheimer's disease by decreasing the production of amyloid beta, decreasing the formation of amyloid plaques, decreasing the formation of neurofibrillary tangles, decreasing the formation of neuritic plaques, decreasing the formation of Senile plaques, decreasing the existing amount of neurofibrillary tangles, decreasing the existing amount of neuritic plaques, decreasing the existing amount of Senile plaques, increasing survival, increasing mental wellbeing, increasing mental function, slowing the decrease of mental function, decreasing dementia, delaying the onset of dementia, improving cognitive skills, decreasing the loss of cognitive skills, improving memory, decreasing the degradation of memory, or extending survival. The term “treating,” and conjugations thereof, include prevention of an injury, pathology, condition, or disease. For example, in some embodiments, treating Bovine spongiform encephalopathy in a cow or bovine includes preventing the onset of Bovine spongiform encephalopathy. For example, in some embodiments, treating Alzheimer's disease in a human includes preventing the onset of Alzheimer's disease.

An “effective amount” is an amount sufficient to accomplish a stated purpose (e.g. achieve the effect for which it is administered, treat a disease, reduce enzyme activity, reduce one or more symptoms of a disease or condition, reduce the amount of a misfolded protein in a cell, reduce the amount of PrP^{Sc} in a cell, reduce the amount of PrP^{C} in a cell, reduce the amount of amyloid beta, reduce the amount of amyloid plaques, reduce the activity of gamma secretase, reduce the activity of beta secretase). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist (e.g. gamma secretase, beta secretase). A “function disrupting amount,” as used herein, refers to the amount of antagonist required to disrupt the function of an enzyme or protein relative to the absence of the antagonist (e.g. gamma secretase, beta secretase). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

The term “contacting” may include allowing two species to react, interact, or physically touch, wherein the two species may be a compound as described herein and a misfolded protein. In some embodiments, the misfolded protein may be a prion protein. In some embodiments, the misfolded protein may be amyloid beta or beta amyloid. In some embodiments, the misfolded protein may be huntingtin. In some embodiments, the misfolded protein may be an alpha synuclein. In some embodiments contacting includes allowing a compound described herein to interact with a protein or enzyme that promotes the formation of misfolded proteins (e.g. gamma secretase, beta secretase). In some embodiments, the protein or enzyme promotes the formation of PrP^{Sc}. In some embodiments, the protein or enzyme increases the amount of PrP^{Sc} or PrP^{C}. In some embodiments, the protein or enzyme decreases the amount of PrP^{Sc}. In some embodiments, the protein or enzyme promotes the formation of amyloid beta. In some embodiments, the protein or enzyme increases the amount of amyloid beta or amyloid plaques.

As defined herein, the term “inhibition”, “inhibit”, “inhibiting” and the like in reference to a protein-inhibitor interaction means negatively affecting (e.g. decreasing) the activity or function of the protein (e.g. decreasing the misfolding or aggregation of proteins) relative to the activity or function of the protein in the absence of the inhibitor. In some embodiments inhibition refers to reduction of a disease or symptoms of disease. In some embodiments, inhibition refers to a reduction in the presence of a disease-related protein (e.g. a prion or another misfolded protein or PrP^{Sc} or PrP^{C} or amyloid beta or amyloid precursor protein). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound that inhibits aggregation of a protein or production of a protein, e.g., by binding, partially or totally blocking stimulation (e.g. production), decreasing, preventing, or delaying activation (e.g. of a protein or enzyme involved in producing a misfolded protein or PrP^{Sc} or amyloid beta, such as gamma secretase or beta secretase), or inactivating, desensitizing, or down-regulating signal transduction or enzymatic activity (e.g. activity responsible for producing a misfolded protein or PrP^{Sc} or amyloid beta, such as gamma secretase or beta secretase). Inhibition may also reduce the amount of a protein by increasing clearance or degradation of the protein (e.g. PrP^{Sc}, amyloid beta, amyloid precursor protein).

The term “modulator” refers to a composition that increases or decreases the level of a target molecule or the function of a target molecule (e.g. a target may be a prion protein and the function in a disease state of a prion protein, such as PrP^{Sc}, may be to aggregate or a target may be a amyloid beta and the function in a disease state of amyloid beta may be to aggregate). In some embodiments, a prion modulator or PrP^{Sc} modulator is a compound that reduces the amount of PrP^{Sc} in a cell or reduces the aggregation of PrP^{Sc} into fibrils, plaques, or bundles. A PrP^{Sc} modulator may reduce or increase an enzyme activity that results in a reduction of the amount of PrP^{Sc} in a cell or results in a reduction of aggregated PrP^{Sc}. In some embodiments, a Prion disease modulator is a compound that reduces the severity of one or more symptoms of a prion disease (e.g. loss of mental function, loss of cognitive function). In some embodiments, an amyloid beta modulator is a compound that reduces the amount of amyloid beta in a patient or reduces the aggregation of amyloid beta into fibrils, plaques, or bundles. An amyloid beta modulator may reduce or increase an enzyme activity that results in a reduction of the amount of amyloid beta in a patient or results in a reduction of aggregated amyloid beta. In some embodiments, an Alzheimer's disease modulator is a compound that reduces the severity of one or more symptoms of Alzheimer's disease (e.g. loss of mental function, loss of cognitive function). A gamma secretase modulator is a composition (e.g. compound described herein) that increases or decreases the level of activity or function of gamma secretase. A gamma secretase inhibitor is a composition (e.g. compound described herein) that decreases the level of activity or function of gamma secretase. A beta secretase modulator is a composition (e.g. compound described herein) that increases or decreases the level of activity or function of beta secretase. A beta secretase inhibitor is a composition (e.g. compound described herein) that decreases the level of activity or function of beta secretase.

“Patient” or “subject in need thereof” refers to a living organism suffering from or prone to a condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is bovine. In some embodiments, a patient is a cow. In some embodiments, a patient is mammal.

“Disease” or “condition” refer to a state of being or health status of a patient or subject capable of being treated with the compounds or methods provided herein. In some embodiments, the disease is a disease related to (e.g. caused by) a protein misfolding and/or protein aggregation (e.g. neurodegenerative diseases, Alzheimer's disease, Parkinson's disease, Huntington's Disease, frontotemporal dementia, Bovine spongiform encephalopathy (BSE), Creutzfeldt-Jakob disease, Gerstmann-Strussler-Scheinker syndrome, kuru, prion disease.) Examples of diseases, disorders, or conditions include, but are not limited to, neurodegenerative diseases, frontotemporal dementia, cancer, cardiovascular disease, hypertension, Syndrome X, depression, anxiety, glaucoma, human immunodeficiency virus (HIV) or acquired immunodeficiency syndrome (AIDS), neurodegeneration, Alzheimer's disease, Parkinson's disease, cognition enhancement, Cushing's Syndrome, Addison's Disease, osteoporosis, frailty, muscle frailty, inflammatory diseases, osteoarthritis, rheumatoid arthritis, asthma and rhinitis, adrenal function-related ailments, viral infection, immunodeficiency, immunomodulation, autoimmune diseases, allergies, wound healing, compulsive behavior, multi-drug resistance, addiction, psychosis, anorexia, cachexia, post-traumatic stress syndrome, post-surgical bone fracture, medical catabolism, major psychotic depression, mild cognitive impairment, psychosis, dementia, hyperglycemia, stress disorders, antipsychotic induced weight gain, delirium, cognitive impairment in depressed patients, cognitive deterioration in individuals with Down's syndrome, psychosis associated with interferon-alpha therapy, chronic pain, pain associated with gastroesophageal reflux disease, postpartum psychosis, postpartum depression, neurological disorders in premature infants, migraine headaches, stroke, aneurysm, brain aneurysm, cerebral aneurysm, brain attack, cerebrovascular accident, ischemia, thrombosis, arterial embolism, hemorrhage, transient ischemic attack, anemia, embolism, systemic hypoperfusion, venous thrombosis, arthritis, reperfusion injury, skin diseases or conditions, acne, acne vulgaris, keratosis pilaris, acute, promyelocytic leukemia, baldness, acne rosacea, harlequin ichthyosis, xeroderma pigmentosum, keratoses, neuroblastoma, fibrodysplasia ossificans progressive, eczema, rosacea, sun damage, wrinkles, or cosmetic conditions. In some instances, “disease” or “condition” refer to cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “neurodegenerative disease” refers to a disease or condition in which the function of a subject's nervous system becomes impaired. Examples of neurodegenerative diseases that may be treated with a compound or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Strussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to

Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “autoimmune disease” refers to a disease or condition in which a subject's immune system irregularly responds to one or more components (e.g. biomolecule, protein, cell, tissue, organ, etc.) of the subject. In some embodiments, an autoimmune disease is a condition in which the subject's immune system irregularly reacts to one or more components of the subject as if such components were not self. Exemplary autoimmune diseases that may be treated with a compound or method provided herein include Acute Disseminated Encephalomyelitis (ADEM), Acute necrotizing hemorrhagic leukoencephalitis, Addison's disease, Agammaglobulinemia, Asthma, Allergic asthma, Allergic rhinitis, Alopecia greata, Amyloidosis, Ankylosing spondylitis, Anti-GBM/Anti-TBM nephritis, Antiphospholipid syndrome (APS), Arthritis, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Axonal & neuronal neuropathies, Balo disease, Behcet's disease, Bullous pemphigoid, Cardiomyopathy, Castleman disease, Celiac sprue, Chagas disease, Chronic inflammatory demyelinating polyneuropathy (CIDP), Chronic recurrent multifocal osteomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic fasciitis, Erythema nodosum, Experimental allergic encephalomyelitis, Evans syndrome, Fibrosing alveolitis, Giant cell arteritis (temporal arteritis), Glomerulonephritis, Goodpasture's syndrome, Graves' disease, Grave's ophthalmopathy, Guillain-Barre syndrome, Hashimoto's encephalitis, Hashimoto's thyroiditis, Hemolytic anemia, Henoch-Schonlein purpura, Herpes gestationis, Hypogammaglobulinemia, Ichthyosis, Idiopathic thrombocytopenic purpura (ITP), IgA nephropathy, IgG4-related sclerosing disease, Immunoregulatory lipoproteins, Inclusion body myositis, Inflammatory bowel disease, Insulin-dependent diabetes (typel), Interstitial cystitis, Juvenile arthritis, Juvenile diabetes, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic, arthritis, Idiopathic pulmonary fibrosis, Pyoderma gangrenous, Pure red cell aplasia, Raynauds phenomenon, Reflex sympathetic dystrophy Reiter's syndrome, Relapsing polychondritis, Restless legs syndrome, Retroperitoneal Fibrosis, Rheumatic fever Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, or Wegener's granulomatosis.

As used herein, the term “inflammatory disease” refers to any disease characterized by abnormal inflammation. Exemplary inflammatory diseases that may be treated with a compound or method provided herein include arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, or allergic asthma.

As used herein, the term “cardiovascular disease” refers to a disease or condition affecting the heart or blood vessels. In embodiments, cardiovascular disease includes diseases caused by or exacerbated by atherosclerosis. Exemplary cardiovascular diseases that may be treated with a compound or method provided herein include Alcoholic cardiomyopathy, Coronary artery disease, Congenital heart disease, Arrhythmogenic right ventricular cardiomyopathy, Restrictive cardiomyopathy, Noncompaction Cardiomyopathy, diabetes mellitus, hypertension, hyperhomocysteinemia, hypercholesterolemia, Atherosclerosis, Ischemic heart disease, Heart failure, Cor pulmonale, Hypertensive heart disease, Left ventricular hypertrophy, Coronary heart disease, (Congestive) heart failure, Hypertensive cardiomyopathy, Cardiac arrhythmias, Inflammatory heart disease, Endocarditis, Inflammatory cardiomegaly, Myocarditis, Valvular heart disease, stroke, or myocardial infarction. In some embodiments, treating a cardiovascular disease includes treating a condition or symptom caused by a cardiovascular disease. A non-limiting example of such a treatment is treating complications due to a myocardial infarction, after the myocardial infarction has occurred.

As used herein, the term “cancer” refers to all types of cancer, neoplasm or malignant tumors found in mammals, including leukemia, carcinomas and sarcomas. Exemplary cancers that may be treated with a compound or method provided herein include cancer of the brain, breast, cervix, colon, head & neck, liver, kidney, lung, non-small cell lung, melanoma, mesothelioma, ovary, sarcoma, stomach, uterus or Medulloblastoma. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “Prion disease” refers to any neurodegenerative disease associated with a misfolded or abnormally folded protein, which is disease-associated when misfolded or abnormally folded, but is not disease-associated when folded in another conformation. The prion protein associated with the prion disease may induce other non-disease-associated prion proteins to become disease-related by inducing abnormal folding. Examples of prion diseases include Bovine spongiform encephalopathy (BSE), Creutzfeldt-Jakob disease, Gerstmann-Strussler-Scheinker syndrome, kuru, chronic wasting disease, and scrapie. The non-disease associated prion protein is sometimes referred to as “PrP^{C}”. The disease-associated prion protein is sometimes referred to as “Prp^{Sc}”.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine. and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

### II. Compounds

In a first aspect is a compound having the formula:

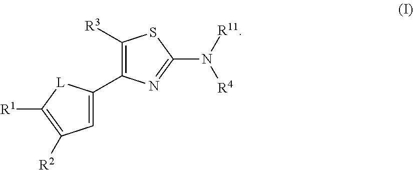

L is —CR⁶═CH—, —S—, or —O—. R⁷, R⁸, R⁹ and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹¹ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —C(O)R¹², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹² is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl substituted or unsubstituted heterocycloalkyl, substituted or unsubstituted aryl, or substituted or unsubstituted heteroaryl. R¹, R², R³, R⁴, and R⁶ are independently hydrogen, halogen, —CX₃, —OCX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol v is independently 1 or 2. The symbol m is independently an integer from 1 to 2. The symbol n is independently an integer from 0 to 4. X is independently —Cl, —Br, —I, or —F. Two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl. Adjacent R¹ and R² substituents may optionally be joined to form a substituted or unsubstituted cycloalkyl, substituted or unsubstituted heterocycloalkyl, substituted or unsubstituted aryl, or substituted or unsubstituted heteroaryl. Adjacent R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl. Adjacent R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, L, X, v, m, n, R¹, R², R³, R⁴, R⁶, R⁷, R⁸, R⁹, R¹⁰, R₁₁, and R¹², are as described in the paragraphs below in a compound of formula (I). In some embodiments, these values are included in any other formula described herein. In some embodiments, adjacent R¹ and R² substituents are joined to form a substituted or unsubstituted heterocycloalkyl, R²⁰-substituted or unsubstituted heterocycloalkyl, or R²³-substituted or unsubstituted heterocycloalkyl. In some embodiments, adjacent R¹ and R² substituents are joined to form a substituted or unsubstituted 2,3-dihydro-1,4-dioxinyl, R²⁰-substituted or unsubstituted 2,3-dihydro-1,4-dioxinyl, or R²³-substituted or unsubstituted 2,3-dihydro-1,4-dioxinyl. In some embodiments, adjacent R¹ and R² substituents are joined to form a substituted or unsubstituted phenyl, R²⁰-substituted or unsubstituted phenyl, or R²³-substituted or unsubstituted phenyl. In some embodiments, adjacent R¹ and R² substituents are joined to form an unsubstituted phenyl. Where each R⁷, R⁸, R⁹, and R¹⁰, X, n, v, and m is different, they may be referred to, for example, as R^{7c}, R^{7d}, R^{7ex}, R^{7f}, R^{7g}, R^{7h}, R⁷ⁱ, R^{8c}, R^{8d}, R^{8e}, R^{8f}, R^{8g}, R^{8h}, R⁸ⁱ, R^{9c}, R^{9d}, R^{9e}, R^{9f}, R^{9g}, R^{9h}, R⁹ⁱ, R^{10c}, R^{10d}, R^{10e}, R^{10f}, R^{10g}, R^{10h}, R¹⁰ⁱ, X^{c}, X^{d}, X^{e}, X^{f}, X^{g}, X^{h}, Xⁱ, n^{c}, n^{d}, n^{e}, n^{f}, n^{g}, n^{h}, nⁱ, v^{c}, v^{d}, v^{e}, v^{f}, v^{g}, V^{h}, vⁱ, m^{c}, m^{d}, m^{e}, m^{f}, m^{g}, m^{h}, mⁱ, and so on, wherein each R^{7c}, R^{7d}, R^{7e}, R^{7f}, R^{7g}, R^{7h}, R⁷ⁱ is defined the same as R⁷, each R^{8c}, R^{8d}, R^{8e}, R^{8f}, R^{8g}, R^{8h}, R⁸ⁱ is defined the same as R⁸, each R^{9c}, R^{9d}, R^{9e}, R^{9f}, R^{9g}, R^{9h}, R⁹ⁱ is defined the same as R⁹, each R^{10c}, R^{10d}, R^{10e}, R^{10f}, R^{10g}, R^{10h}, R¹⁰ⁱ is defined the same as R¹⁰, each X^{c}, X^{d}, X^{e}, X^{f}, X^{g}, X^{h}, Xⁱ is defined the same as X, each n^{c}, n^{d}, n^{e}, n^{f}, n^{g}, n^{h}, nⁱ is defined the same as n, each v^{c}, v^{d}, v^{f}, v^{g}, v^{h}, vⁱ is defined the same as v, each m^{c}, m^{d}, m^{e}, m^{f}, m^{g}, m^{h}, mⁱ is defined the same as m. In some embodiments, R² is defined by R^{7c}, R^{8c}, R^{9c}, R^{10c}, X^{c}, n^{c}, v^{c}, and m^{c}. In some embodiments, R³ is defined by R^{7d}, R^{8d}, R^{9d}, R^{10d}, X^{d}, n^{d}, v^{d}, and m^{d}. In some embodiments, R⁴ is defined by R^{7e}, R^{8e}, R^{9e}, R^{10e}, X^{e}, n^{e}, v^{e}, and m^{e}. In some embodiments, R⁶ is defined by R^{7f}, R^{8f}, R^{9f}, R^{10f}, X^{f}, n^{f}, v^{f}, and m^{f}. In some embodiments, R¹ is defined by R^{7g}, R^{8g}, R^{9g}, R^{10g}, X^{g}, n^{g}, v^{g}, and m^{g}. In some embodiments, R^{1A} is defined by R^{7h}, R^{8h}, R^{9h}, R^{10h}, X^{h}, V^{h}, and m^{h}. In some embodiments, R^{1B} is defined by R⁷ⁱ, R⁸ⁱ, R⁹ⁱ, R¹⁰ⁱ, Xⁱ, nⁱ, vⁱ, and mⁱ. Where c, d, e, f, g, h, and i denote substituents of R², R³, R⁴, R⁶, R¹¹, R^{1A}, and R^{1B} respectively.

In some embodiments, L is —CR⁶═CH—. In some embodiments, L is —S—. In some embodiments, L is —O—. In some embodiments, R⁶ is hydrogen, —CN, or —OR¹⁰. In some embodiments, R⁶ is hydrogen. In some embodiments, R⁶ is —CN. In some embodiments, R⁶ is —OR¹⁰.

In some embodiments, R¹ is substituted or unsubstituted cycloalkyl, substituted or unsubstituted heterocycloalkyl, substituted or unsubstituted aryl, or substituted or unsubstituted heteroaryl. In some embodiments, R¹ is substituted or unsubstituted aryl or substituted or unsubstituted heteroaryl. In some embodiments, R¹ is substituted or unsubstituted heteroaryl. In some embodiments, R¹ is substituted heteroaryl. In some embodiments, R¹ is R²⁰-substituted heteroaryl. In some embodiments, R²⁰ is independently F, —OCH(CH₃)₂, —OCH₂CH₂OCH₃, —OCH₃, —N(CH₃)₂, methyl, —OCH₂CN, pyridinyl (pyridyl), piperazinyl, or 1-(4-methyl)piperazinyl. In some embodiments, R¹ is unsubstituted heteroaryl. In some embodiments, R¹ is pyridinyl (pyridyl). In some embodiments, R¹ is 2-pyridinyl (2-pyridyl). In some embodiments, R¹ is 3-pyridinyl (3-pyridyl). In some embodiments, R¹ is 4-pyridyl. In some embodiments, R¹ is substituted or unsubstituted aryl. In some embodiments, R¹ is unsubstituted aryl. In some embodiments, R¹ is phenyl. In some embodiments, R¹ is R²⁰-substituted aryl. In some embodiments, R²⁰ is independently substituted with F, —OCH(CH₃)₂, —OCH₂CH₂OCH₃, —OCH₃, —N(CH₃)₂, methyl, —OCH₂CN, pyridyl, piperazinyl, or 1-(4-methyl)piperazinyl. In some embodiments, R¹ is substituted aryl or substituted heteroaryl. In some embodiments, R¹ is aryl substituted at the para position relative to the L-containing ring (i.e. ring with R² substituent). In some embodiments, R¹ is aryl substituted at the meta position relative to the L-containing ring. In some embodiments, R¹ is heteroaryl substituted at the para position relative to the L-containing ring. In some embodiments, R¹ is heteroaryl substituted at the meta position relative to the L-containing ring.

In some embodiments, R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹ is substituted or unsubstituted alkyl. In some embodiments, R¹ is substituted or unsubstituted heteroalkyl. In some embodiments, R¹ is substituted or unsubstituted cycloalkyl. In some embodiments, R¹ is substituted or unsubstituted heterocycloalkyl. In some embodiments, R¹ is substituted or unsubstituted aryl. In some embodiments, R¹ is substituted or unsubstituted heteroaryl. In some embodiments, R¹ is unsubstituted alkyl. In some embodiments, R¹ is unsubstituted heteroalkyl. In some embodiments, R¹ is unsubstituted cycloalkyl. In some embodiments, R¹ is unsubstituted heterocycloalkyl. In some embodiments, R¹ is unsubstituted aryl. In some embodiments, R¹ is unsubstituted heteroaryl. In some embodiments, R¹ is substituted alkyl. In some embodiments, R¹ is substituted heteroalkyl. In some embodiments, R¹ is substituted cycloalkyl. In some embodiments, R¹ is substituted heterocycloalkyl. In some embodiments, R¹ is substituted aryl. In some embodiments, R¹ is substituted heteroaryl. In some embodiments, R¹ is R²⁰-substituted alkyl. In some embodiments, R¹ is R²⁰-substituted heteroalkyl. In some embodiments, R¹ is R²⁰-substituted cycloalkyl. In some embodiments, R¹ is R²⁰-substituted heterocycloalkyl. In some embodiments, R¹ is R²⁰-substituted aryl. In some embodiments, R¹ is R²⁰-substituted heteroaryl. In some embodiments, R¹ is substituted or unsubstituted phenyl. In some embodiments, R¹ is R²⁰-substituted or unsubstituted phenyl. In some embodiments, R¹ is substituted phenyl. In some embodiments, R¹ is unsubstituted phenyl. In some embodiments, R¹ is R²⁰-substituted phenyl. In some embodiments, R¹ is substituted or unsubstituted thienyl. In some embodiments, R¹ is R²⁰-substituted or unsubstituted thienyl. In some embodiments, R¹ is substituted thienyl. In some embodiments, R¹ is unsubstituted thienyl. In some embodiments, R¹ is R²⁰-substituted thienyl. In some embodiments, R¹ is substituted or unsubstituted 4,5,6,7-tetrahydrobenzo[b]thienyl. In some embodiments, R¹ is R²⁰-substituted or unsubstituted 4,5,6,7-tetrahydrobenzo[b]thienyl. In some embodiments, R¹ is substituted 4,5,6,7-tetrahydrobenzo[b]thienyl. In some embodiments, R¹ is unsubstituted 4,5,6,7-tetrahydrobenzo[b]thienyl. In some embodiments, R¹ is R²⁰-substituted 4,5,6,7-tetrahydrobenzo[b]thienyl. In some embodiments, R¹ is substituted or unsubstituted pyridyl. In some embodiments, R¹ is R²⁰-substituted or unsubstituted pyridyl. In some embodiments, R¹ is substituted pyridyl. In some embodiments, R¹ is unsubstituted pyridyl. In some embodiments, R¹ is R²⁰-substituted pyridyl. In some embodiments, R¹ is substituted or unsubstituted 1,3,4-thiadiazolyl. In some embodiments, R¹ is R²⁰-substituted or unsubstituted 1,3,4-thiadiazolyl. In some embodiments, R¹ is substituted 1,3,4-thiadiazolyl. In some embodiments, R¹ is unsubstituted 1,3,4-thiadiazolyl. In some embodiments, R¹ is R²⁰-substituted 1,3,4-thiadiazolyl. In some embodiments, R¹ is substituted or unsubstituted piperidinyl. In some embodiments, R¹ is R²⁰-substituted or unsubstituted piperidinyl. In some embodiments, R¹ is substituted piperidinyl. In some embodiments, R¹ is unsubstituted piperidinyl. In some embodiments, R¹ is R²⁰-substituted piperidinyl. In some embodiments, R¹ is substituted or unsubstituted piperazinyl. In some embodiments, R¹ is R²⁰-substituted or unsubstituted piperazinyl. In some embodiments, R¹ is substituted piperazinyl. In some embodiments, R¹ is unsubstituted piperazinyl. In some embodiments, R¹ is R²⁰-substituted piperazinyl. In some embodiments, R¹ is substituted or unsubstituted oxazolyl. In some embodiments, R¹ is R²⁰-substituted or unsubstituted oxazolyl. In some embodiments, R¹ is substituted oxazolyl. In some embodiments, R¹ is unsubstituted oxazolyl. In some embodiments, R¹ is R²⁰-substituted oxazolyl. In some embodiments, R¹ is substituted or unsubstituted thiazolyl. In some embodiments, R¹ is R²⁰-substituted or unsubstituted thiazolyl. In some embodiments, R¹ is substituted thiazolyl. In some embodiments, R¹ is unsubstituted thiazolyl. In some embodiments, R¹ is R²⁰-substituted thiazolyl. In some embodiments, R¹ is R²⁰-substituted or unsubstituted benzo[d]oxazolyl. In some embodiments, R¹ is R²⁰-substituted benzo[d]oxazolyl. In some embodiments, R¹ is unsubstituted benzo[d]oxazolyl. In some embodiments, R¹ is substituted or unsubstituted tetrahydrothienyl. In some embodiments, R¹ is R²⁰-substituted or unsubstituted tetrahydrothienyl. In some embodiments, R¹ is substituted tetrahydrothienyl. In some embodiments, R¹ is unsubstituted tetrahydrothienyl. In some embodiments, R¹ is R²⁰-substituted tetrahydrothienyl. In some embodiments, R¹ is substituted or unsubstituted 2,3-dihydro-1H-pyrazolyl. In some embodiments, R¹ is R²⁰-substituted or unsubstituted 2,3-dihydro-1H-pyrazolyl. In some embodiments, R¹ is substituted 2,3-dihydro-1H-pyrazolyl. In some embodiments, R¹ is unsubstituted 2,3-dihydro-1H-pyrazolyl. In some embodiments, R¹ is R²⁰-substituted 2,3-dihydro-1H-pyrazolyl. In some embodiments, R¹ is substituted or unsubstituted (C₁-C₄) alkyl. In some embodiments, R¹ is R²⁰-substituted or unsubstituted (C₁-C₄) alkyl. In some embodiments, R¹ is substituted (C₁-C₄) alkyl. In some embodiments, R¹ is unsubstituted (C₁-C₄) alkyl. In some embodiments, R¹ is R²⁰-substituted (C₁-C₄) alkyl. In some embodiments, R¹ is substituted or unsubstituted ethyl. In some embodiments, R¹ is R²⁰-substituted or unsubstituted ethyl. In some embodiments, R¹ is substituted ethyl. In some embodiments, R¹ is unsubstituted ethyl. In some embodiments, R¹ is R²⁰-substituted ethyl.

In some embodiments, R¹ is R²⁰-substituted or unsubstituted imidazolyl. In some embodiments, R¹ is substituted imidazolyl. In some embodiments, R¹ is unsubstituted imidazolyl. In some embodiments, R¹ is R²⁰-substituted or unsubstituted 4H-1,2,4-triazolyl. In some embodiments, R¹ is substituted 4H-1,2,4-triazolyl. In some embodiments, R¹ is unsubstituted 4H-1,2,4-triazolyl. In some embodiments, R¹ is R²⁰-substituted or unsubstituted triazolyl. In some embodiments, R¹ is substituted triazolyl. In some embodiments, R¹ is unsubstituted triazolyl. In some embodiments, R¹ is substituted or unsubstituted naphthyl. In some embodiments, R¹ is substituted or unsubstituted furanyl. In some embodiments, R¹ is substituted or unsubstituted quinolinyl. In some embodiments, R¹ is unsubstituted naphthyl. In some embodiments, R¹ is unsubstituted furanyl. In some embodiments, R¹ is unsubstituted quinolinyl. In some embodiments, R¹ is substituted naphthyl. In some embodiments, R¹ is substituted furanyl. In some embodiments, R¹ is substituted quinolinyl. In some embodiments, R¹ is R²⁰-substituted naphthyl. In some embodiments, R¹ is R²⁰-substituted furanyl. In some embodiments, R¹ is R²⁰-substituted quinolinyl. In some embodiments, R¹ is substituted or unsubstituted morpholinyl. In some embodiments, R¹ is R²⁰-substituted or unsubstituted morpholinyl. In some embodiments, R¹ is substituted morpholinyl. In some embodiments, R¹ is unsubstituted morpholinyl. In some embodiments, R¹ is R²⁰-substituted morpholinyl. In some embodiments, R¹ is substituted or unsubstituted piperazinyl. In some embodiments, R¹ is R²⁰-substituted or unsubstituted piperazinyl. In some embodiments, R¹ is substituted piperazinyl. In some embodiments, R¹ is unsubstituted piperazinyl. In some embodiments, R¹ is R²⁰-substituted piperazinyl. In some embodiments, R¹ is substituted or unsubstituted pyrazolyl (e.g. 1H-pyrazolyl). In some embodiments, R¹ is R²⁰-substituted or unsubstituted pyrazolyl (e.g. 1H-pyrazolyl). In some embodiments, R¹ is substituted pyrazolyl (e.g. 1H-pyrazolyl). In some embodiments, R¹ is unsubstituted pyrazolyl (e.g. 1H-pyrazolyl). In some embodiments, R¹ is R²⁰-substituted pyrazolyl (e.g. 1H-pyrazolyl). In some embodiments, R¹ is substituted or unsubstituted isoxazolyl. In some embodiments, R¹ is R²⁰-substituted or unsubstituted isoxazolyl. In some embodiments, R¹ is substituted isoxazolyl. In some embodiments, R¹ is unsubstituted isoxazolyl. In some embodiments, R¹ is R²⁰-substituted isoxazolyl.

In some embodiments, R¹ is substituted with one R²⁰. In some embodiments, R¹ is substituted with two optionally different R²⁰. In some embodiments, R¹ is substituted with three optionally different R²⁰. In some embodiments, R¹ is substituted with four optionally different R²⁰.

In some embodiments, two adjacent R²⁰ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁰ substituents are joined to form a substituted cycloalkyl. In some embodiments, two adjacent R²⁰ substituents are joined to form an unsubstituted cycloalkyl. In some embodiments, two adjacent R²⁰ substituents are joined to form a substituted heterocycloalkyl. In some embodiments, two adjacent R²⁰ substituents are joined to form an unsubstituted heterocycloalkyl. In some embodiments, two adjacent R²⁰ substituents are joined to form a substituted aryl. In some embodiments, two adjacent R²⁰ substituents are joined to form an unsubstituted aryl. In some embodiments, two adjacent R²⁰ substituents are joined to form a substituted heteroaryl. In some embodiments, two adjacent R²⁰ substituents are joined to form an unsubstituted heteroaryl.

In some embodiments, R²⁰ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R²¹-substituted or unsubstituted piperidinyl, R²¹-substituted or unsubstituted piperazinyl, R²¹-substituted or unsubstituted thiazolyl, R²¹-substituted or unsubstituted oxazolyl, R²¹-substituted or unsubstituted phenyl, R²¹-substituted or unsubstituted thienyl, R²¹-substituted or unsubstituted 4,5,6,7-tetrahydrobenzo[b]thienyl, R²¹— substituted or unsubstituted pyridyl, or R²¹-substituted or unsubstituted 1,3,4-thiadiazolyl. In some embodiments, R²⁰ is R²¹-substituted substituted or unsubstituted benzo[d]oxazolyl. In some embodiments, R²⁰ is R²¹-substituted or unsubstituted imidazolyl. In some embodiments, R²⁰ is R²¹-substituted imidazolyl. In some embodiments, R²⁰ is unsubstituted imidazolyl. In some embodiments, R²⁰ is R²¹-substituted or unsubstituted 4H-1,2,4-triazolyl. In some embodiments, R²⁰ is R²¹-substituted 4H-1,2,4-triazolyl. In some embodiments, R²⁰ is unsubstituted 4H-1,2,4-triazolyl. In some embodiments, R²⁰ is R²¹-substituted substituted or unsubstituted triazolyl. In some embodiments, R²⁰ is R²¹-substituted triazolyl. In some embodiments, R²⁰ is unsubstituted triazolyl. In some embodiments, R²⁰ is independently R²¹-substituted or unsubstituted alkyl, R²¹-substituted or unsubstituted heteroalkyl, R²¹-substituted or unsubstituted cycloalkyl, R²¹ substituted or unsubstituted heterocycloalkyl, R²¹-substituted or unsubstituted aryl, or R²¹-substituted or unsubstituted heteroaryl. In some embodiments, R²⁰ is independently R²¹-substituted alkyl. In some embodiments, R²⁰ is unsubstituted alkyl. In some embodiments, R²⁰ is R²¹-substituted heteroalkyl. In some embodiments, R²⁰ is unsubstituted heteroalkyl. In some embodiments, R²⁰ is R²¹-substituted cycloalkyl. In some embodiments, R²⁰ is unsubstituted cycloalkyl. In some embodiments, R²⁰ is R²¹-substituted substituted heterocycloalkyl. In some embodiments, R²⁰ is unsubstituted heterocycloalkyl. In some embodiments, substituted aryl. In some embodiments, R²⁰ is R²⁰ is R²¹-substituted R²¹—unsubstituted aryl. In some embodiments, R²⁰ is R²¹-substituted heteroaryl. In some embodiments, R²⁰ is unsubstituted heteroaryl. In some embodiments, R²⁰ is substituted with one R²¹. In some embodiments, R²⁰ is substituted with two optionally different R²¹. In some embodiments, R²⁰ is substituted with three optionally different R²¹. In some embodiments, R²⁰ is substituted with four optionally different R²¹. In some embodiments, R²⁰ is independently oxo. In some embodiments, R²⁰ is independently —Br. In some embodiments, R²⁰ is independently —F. In some embodiments, R²⁰ is independently —Cl. In some embodiments, R²⁰ is independently —I. In some embodiments, R²⁰ is independently —CH₃. In some embodiments, R²⁰ is independently —OCH₃. In some embodiments, R²⁰ is independently (C₁-C₄) alkyl. In some embodiments, R²⁰ is independently (C₁-C₈) alkyl. In some embodiments, R²⁰ is independently (C₇-C₁₀) alkyl. In some embodiments, R²⁰ is independently (C₆-C₁₂) alkyl. In some embodiments, R²⁰ is independently phenyl. In some embodiments, R²⁰ is independently —OH. In some embodiments, R²⁰ is independently —CF₃.

In some embodiments, two adjacent R²¹ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²¹ substituents are joined to form a substituted cycloalkyl. In some embodiments, two adjacent R²¹ substituents are joined to form an unsubstituted cycloalkyl. In some embodiments, two adjacent R²¹ substituents are joined to form a substituted heterocycloalkyl. In some embodiments, two adjacent R²¹ substituents are joined to form an unsubstituted heterocycloalkyl. In some embodiments, two adjacent R²¹ substituents are joined to form a substituted aryl. In some embodiments, two adjacent R²¹ substituents are joined to form an unsubstituted aryl. In some embodiments, two adjacent R²¹ substituents are joined to form a substituted heteroaryl. In some embodiments, two adjacent R²¹ substituents are joined to form an unsubstituted heteroaryl.

In some embodiments, R²¹ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R²¹ is R²²-substituted or unsubstituted alkyl, R²²-substituted or unsubstituted heteroalkyl, R²²-substituted or unsubstituted cycloalkyl, R²²-substituted or unsubstituted heterocycloalkyl, R²²-substituted or unsubstituted aryl, or R²²-substituted or unsubstituted heteroaryl. In some embodiments, R²¹ is CH₃. In some embodiments, R²¹ is independently R²²-substituted or unsubstituted piperidinyl, R²²-substituted or unsubstituted piperazinyl, R²²-substituted or unsubstituted thiazolyl, R²²-substituted or unsubstituted oxazolyl, R²²-substituted or unsubstituted phenyl, R²²-substituted or unsubstituted thienyl, R²²-substituted or unsubstituted 4,5,6,7-tetrahydrobenzo[b]thienyl, R²²-substituted or unsubstituted pyridyl, or R²²-substituted or unsubstituted 1,3,4-thiadiazolyl. In some embodiments, R²¹ is R²²-substituted or unsubstituted benzo[d]oxazolyl. In some embodiments, R²¹ is R²²-substituted or unsubstituted imidazolyl. In some embodiments, R²¹ is R²²-substituted imidazolyl. In some embodiments, R²¹ is unsubstituted imidazolyl. In some embodiments, R²¹ is R²²-substituted or unsubstituted 4H-1,2,4-triazolyl. In some embodiments, R²¹ is R²²-substituted 4H-1,2,4-triazolyl. In some embodiments, R²¹ is unsubstituted 4H-1,2,4-triazolyl. In some embodiments, R²¹ is R²²-substituted or unsubstituted triazolyl. In some embodiments, R²¹ is R²²-substituted triazolyl. In some embodiments, R²¹ is unsubstituted triazolyl. In some embodiments, R²¹ is independently R²²-substituted or unsubstituted alkyl, R²²-substituted or unsubstituted heteroalkyl, R²²-substituted or unsubstituted cycloalkyl, R²² substituted or unsubstituted heterocycloalkyl, R²²-substituted or unsubstituted aryl, or R²²-substituted or unsubstituted heteroaryl. In some embodiments, R²¹ is independently R²²-substituted alkyl. In some embodiments, R²¹ is unsubstituted alkyl. In some embodiments, R²¹ is R²²-substituted heteroalkyl. In some embodiments, R²¹ is unsubstituted heteroalkyl. In some embodiments, R²¹ is R²²-substituted cycloalkyl. In some embodiments, R²¹ is unsubstituted cycloalkyl. In some embodiments, R²¹ is R²²-substituted heterocycloalkyl. In some embodiments, R²¹ is unsubstituted heterocycloalkyl. In some embodiments, R²¹ is R²²-substituted aryl. In some embodiments, R²¹ is unsubstituted aryl. In some embodiments, R²¹ is R²²-substituted heteroaryl. In some embodiments, R²¹ is unsubstituted heteroaryl. In some embodiments, R²¹ is substituted with one R²². In some embodiments, R²¹ is substituted with two optionally different R²². In some embodiments, R²¹ is substituted with three optionally different R²². In some embodiments, R²¹ is substituted with four optionally different R²². In some embodiments, R²¹ is independently oxo. In some embodiments, R²¹ is independently —Br. In some embodiments, R²¹ is independently —F. In some embodiments, R²¹ is independently —Cl. In some embodiments, R²¹ is independently —I. In some embodiments, R²¹ is independently —CH₃. In some embodiments, R²¹ is independently —OCH₃. In some embodiments, R²¹ is independently (C₁-C₄) alkyl. In some embodiments, R²¹ is independently (C₁-C₈) alkyl. In some embodiments, R²¹ is independently (C₇-C₁₀) alkyl. In some embodiments, R²¹ is independently (C₆-C₁₂) alkyl. In some embodiments, R²¹ is independently phenyl. In some embodiments, R²¹ is independently —OH. In some embodiments, R²¹ is independently —CF₃. In some embodiments, R²¹ is R²²-substituted or unsubstituted benzo[d]oxazolyl. In some embodiments, R²¹ is R²²-substituted benzo[d]oxazolyl. In some embodiments, R²¹ is unsubstituted benzo[d]oxazolyl. In some embodiments, R²¹ is R²²-substituted or unsubstituted imidazolyl. In some embodiments, R²¹ is R²²-substituted imidazolyl. In some embodiments, R²¹ is unsubstituted imidazolyl. In some embodiments, R²¹ is R²²-substituted or unsubstituted 4H-1,2,4-triazolyl. In some embodiments, R²¹ is R²²-substituted 4H-1,2,4-triazolyl. In some embodiments, R²¹ is unsubstituted 4H-1,2,4-triazolyl. In some embodiments, R²¹ is R²²— substituted or unsubstituted triazolyl. In some embodiments, R²¹ is R²²-substituted triazolyl. In some embodiments, R²¹ is unsubstituted triazolyl. In some embodiments, R²¹ is R²²-substituted or unsubstituted (C₁-C₄) alkyl. In some embodiments, R²¹ is 2-propyl. In some embodiments, R²¹ is methyl. In some embodiments, R²¹ is ethyl. In some embodiments, R²¹ is propyl. In some embodiments, R²¹ is butyl. In some embodiments, R²¹ is cyclopropyl. In some embodiments, R²¹ is cyclobutyl.

In some embodiments, R² is hydrogen. In some embodiments, R² is —OCH₃. In some embodiments, R² is substituted or unsubstituted alkyl. In some embodiments, R² is substituted alkyl. In some embodiments, R² is unsubstituted alkyl. In some embodiments, R² is substituted or unsubstituted heteroalkyl. In some embodiments, R² is substituted heteroalkyl. In some embodiments, R² is unsubstituted heteroalkyl.

In some embodiments, R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R² is substituted or unsubstituted alkyl. In some embodiments, R² is substituted or unsubstituted heteroalkyl. In some embodiments, R² is substituted or unsubstituted cycloalkyl. In some embodiments, R² is substituted or unsubstituted heterocycloalkyl. In some embodiments, R² is substituted or unsubstituted aryl. In some embodiments, R² is substituted or unsubstituted heteroaryl. In some embodiments, R² is unsubstituted alkyl. In some embodiments, R² is unsubstituted heteroalkyl. In some embodiments, R² is unsubstituted cycloalkyl. In some embodiments, R² is unsubstituted heterocycloalkyl. In some embodiments, R² is unsubstituted aryl. In some embodiments, R² is unsubstituted heteroaryl. In some embodiments, R² is substituted alkyl. In some embodiments, R² is substituted heteroalkyl. In some embodiments, R² is substituted cycloalkyl. In some embodiments, R² is substituted heterocycloalkyl. In some embodiments, R² is substituted aryl. In some embodiments, R² is substituted heteroaryl. In some embodiments, R² is R²³-substituted alkyl. In some embodiments, R² is R²³-substituted heteroalkyl. In some embodiments, R² is R²³-substituted cycloalkyl. In some embodiments, R² is R²³-substituted heterocycloalkyl. In some embodiments, R² is R²³-substituted aryl. In some embodiments, R² is R²³-substituted heteroaryl. In some embodiments, R² is substituted or unsubstituted phenyl. In some embodiments, R² is R²³-substituted or unsubstituted phenyl. In some embodiments, R² is substituted phenyl. In some embodiments, R² is unsubstituted phenyl. In some embodiments, R² is R²³-substituted phenyl. In some embodiments, R² is substituted or unsubstituted thienyl. In some embodiments, R² is R²³-substituted or unsubstituted thienyl. In some embodiments, R² is substituted thienyl. In some embodiments, R² is unsubstituted thienyl. In some embodiments, R² is R²³-substituted thienyl. In some embodiments, R² is substituted or unsubstituted 4,5,6,7-tetrahydrobenzo[b]thienyl. In some embodiments, R² is R²³-substituted or unsubstituted 4,5,6,7-tetrahydrobenzo[b]thienyl. In some embodiments, R² is substituted 4,5,6,7-tetrahydrobenzo[b]thienyl. In some embodiments, R² is unsubstituted 4,5,6,7-tetrahydrobenzo[b]thienyl. In some embodiments, R² is R²³-substituted 4,5,6,7-tetrahydrobenzo[b]thienyl. In some embodiments, R² is substituted or unsubstituted pyridyl. In some embodiments, R² is R²³-substituted or unsubstituted pyridyl. In some embodiments, R² is substituted pyridyl. In some embodiments, R² is unsubstituted pyridyl. In some embodiments, R² is R²³-substituted pyridyl. In some embodiments, R² is substituted or unsubstituted 1,3,4-thiadiazolyl. In some embodiments, R² is R²³-substituted or unsubstituted 1,3,4-thiadiazolyl. In some embodiments, R² is substituted 1,3,4-thiadiazolyl. In some embodiments, R² is unsubstituted 1,3,4-thiadiazolyl. In some embodiments, R² is R²³-substituted 1,3,4-thiadiazolyl. In some embodiments, R² is substituted or unsubstituted piperidinyl. In some embodiments, R² is R²³-substituted or unsubstituted piperidinyl. In some embodiments, R² is substituted piperidinyl. In some embodiments, R² is unsubstituted piperidinyl. In some embodiments, R² is R²³-substituted piperidinyl. In some embodiments, R² is substituted or unsubstituted piperazinyl. In some embodiments, R² is R²³-substituted or unsubstituted piperazinyl. In some embodiments, R² is substituted piperazinyl. In some embodiments, R² is unsubstituted piperazinyl. In some embodiments, R² is R²³-substituted piperazinyl. In some embodiments, R² is substituted or unsubstituted oxazolyl. In some embodiments, R² is R²³-substituted or unsubstituted oxazolyl. In some embodiments, R² is substituted oxazolyl. In some embodiments, R² is unsubstituted oxazolyl. In some embodiments, R² is R²³-substituted oxazolyl. In some embodiments, R² is substituted or unsubstituted thiazolyl. In some embodiments, R² is R²³-substituted or unsubstituted thiazolyl. In some embodiments, R² is substituted thiazolyl. In some embodiments, R² is unsubstituted thiazolyl. In some embodiments, R² is R²³-substituted thiazolyl. In some embodiments, R² is R²³-substituted or unsubstituted benzo[d]oxazolyl. In some embodiments, R² is R²³-substituted benzo[d]oxazolyl. In some embodiments, R² is unsubstituted benzo[d]oxazolyl. In some embodiments, R² is substituted or unsubstituted tetrahydrothienyl. In some embodiments, R² is R²³-substituted or unsubstituted tetrahydrothienyl. In some embodiments, R² is substituted tetrahydrothienyl. In some embodiments, R² is unsubstituted tetrahydrothienyl. In some embodiments, R² is R²³-substituted tetrahydrothienyl. In some embodiments, R² is substituted or unsubstituted 2,3-dihydro-1H-pyrazolyl. In some embodiments, R² is R²³-substituted or unsubstituted 2,3-dihydro-1H-pyrazolyl. In some embodiments, R² is substituted 2,3-dihydro-1H-pyrazolyl. In some embodiments, R² is unsubstituted 2,3-dihydro-1H-pyrazolyl. In some embodiments, R² is R²³-substituted 2,3-dihydro-1H-pyrazolyl. In some embodiments, R² is substituted or unsubstituted (C₁-C₄) alkyl. In some embodiments, R² is R²³-substituted or unsubstituted (C₁-C₄) alkyl. In some embodiments, R² is substituted (C₁-C₄) alkyl. In some embodiments, R² is unsubstituted (C₁-C₄) alkyl. In some embodiments, R² is R²³-substituted (C₁-C₄) alkyl. In some embodiments, R² is substituted or unsubstituted ethyl. In some embodiments, R² is R²³-substituted or unsubstituted ethyl. In some embodiments, R² is substituted ethyl. In some embodiments, R² is unsubstituted ethyl. In some embodiments, R² is R²³-substituted ethyl.

In some embodiments, R² is R²³-substituted or unsubstituted imidazolyl. In some embodiments, R² is substituted imidazolyl. In some embodiments, R² is unsubstituted imidazolyl. In some embodiments, R² is R²³-substituted or unsubstituted 4H-1,2,4-triazolyl. In some embodiments, R² is substituted 4H-1,2,4-triazolyl. In some embodiments, R² is unsubstituted 4H-1,2,4-triazolyl. In some embodiments, R² is R²³-substituted or unsubstituted triazolyl. In some embodiments, R² is substituted triazolyl. In some embodiments, R² is unsubstituted triazolyl. In some embodiments, R² is substituted or unsubstituted naphthyl. In some embodiments, R² is substituted or unsubstituted furanyl. In some embodiments, R² is substituted or unsubstituted quinolinyl. In some embodiments, R² is unsubstituted naphthyl. In some embodiments, R² is unsubstituted furanyl. In some embodiments, R² is unsubstituted quinolinyl. In some embodiments, R² is substituted naphthyl. In some embodiments, R² is substituted furanyl. In some embodiments, R² is substituted quinolinyl. In some embodiments, R² is R²³-substituted naphthyl. In some embodiments, R² is R²³-substituted furanyl. In some embodiments, R² is R²³-substituted quinolinyl. In some embodiments, R² is substituted or unsubstituted morpholinyl. In some embodiments, R² is R²³-substituted or unsubstituted morpholinyl. In some embodiments, R² is substituted morpholinyl. In some embodiments, R² is unsubstituted morpholinyl. In some embodiments, R² is R²³-substituted morpholinyl. In some embodiments, R² is substituted or unsubstituted piperazinyl. In some embodiments, R² is R²³-substituted or unsubstituted piperazinyl. In some embodiments, R² is substituted piperazinyl. In some embodiments, R² is unsubstituted piperazinyl. In some embodiments, R² is R²³-substituted piperazinyl. In some embodiments, R² is substituted or unsubstituted pyrazolyl (e.g. 1H-pyrazolyl). In some embodiments, R² is R²³-substituted or unsubstituted pyrazolyl (e.g. 1H-pyrazolyl). In some embodiments, R² is substituted pyrazolyl (e.g. 1H-pyrazolyl). In some embodiments, R² is unsubstituted pyrazolyl (e.g. 1H-pyrazolyl). In some embodiments, R² is R²³-substituted pyrazolyl (e.g. 1H-pyrazolyl). In some embodiments, R² is substituted or unsubstituted isoxazolyl. In some embodiments, R² is R²³-substituted or unsubstituted isoxazolyl. In some embodiments, R² is substituted isoxazolyl. In some embodiments, R² is unsubstituted isoxazolyl. In some embodiments, R² is R²³-substituted isoxazolyl.

In some embodiments, R² is substituted with one R²³. In some embodiments, R² is substituted with two optionally different R²³. In some embodiments, R² is substituted with three optionally different R²³. In some embodiments, R² is substituted with four optionally different R²³. In some embodiments, two adjacent R²³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²³ substituents are joined to form a substituted cycloalkyl. In some embodiments, two adjacent R²³ substituents are joined to form an unsubstituted cycloalkyl. In some embodiments, two adjacent R²³ substituents are joined to form a substituted heterocycloalkyl. In some embodiments, two adjacent R²³ substituents are joined to form an unsubstituted heterocycloalkyl. In some embodiments, two adjacent R²³ substituents are joined to form a substituted aryl. In some embodiments, two adjacent R²³ substituents are joined to form an unsubstituted aryl. In some embodiments, two adjacent R²³ substituents are joined to form a substituted heteroaryl. In some embodiments, two adjacent R²³ substituents are joined to form an unsubstituted heteroaryl.

In some embodiments, R²³ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R²⁴-substituted or unsubstituted piperidinyl, R²⁴-substituted or unsubstituted piperazinyl, R²⁴-substituted or unsubstituted thiazolyl, R²⁴-substituted or unsubstituted oxazolyl, R²⁴-substituted or unsubstituted phenyl, R²⁴-substituted or unsubstituted thienyl, R²⁴-substituted or unsubstituted 4,5,6,7-tetrahydrobenzo[b]thienyl, R²⁴-substituted or unsubstituted pyridyl, or R²⁴-substituted or unsubstituted 1,3,4-thiadiazolyl. In some embodiments, R²³ is R²⁴-substituted or unsubstituted benzo[d]oxazolyl. In some embodiments, R²³ is R²⁴-substituted or unsubstituted imidazolyl. In some embodiments, R²³ is R²⁴-substituted imidazolyl. In some embodiments, R²³ is unsubstituted imidazolyl. In some embodiments, R²³ is R²⁴-substituted or unsubstituted 4H-1,2,4-triazolyl. In some embodiments, R²³ is R²⁴-substituted 4H-1,2,4-triazolyl. In some embodiments, R²³ is unsubstituted 4H-1,2,4-triazolyl. In some embodiments, R²³ is R²⁴-substituted or unsubstituted triazolyl. In some embodiments, R²³ is R²⁴-substituted triazolyl. In some embodiments, R²³ is unsubstituted triazolyl. In some embodiments, R²³ is independently R²⁴-substituted or unsubstituted alkyl, R²⁴-substituted or unsubstituted heteroalkyl, R²⁴-substituted or unsubstituted cycloalkyl, R²⁴ substituted or unsubstituted heterocycloalkyl, R²⁴-substituted or unsubstituted aryl, or R²⁴-substituted or unsubstituted heteroaryl. In some embodiments, R²³ is independently R²⁴-substituted alkyl. In some embodiments, R²³ is unsubstituted alkyl. In some embodiments, R²³ is R²⁴-substituted heteroalkyl. In some embodiments, R²³ is unsubstituted heteroalkyl. In some embodiments, R²³ is R²⁴-substituted cycloalkyl. In some embodiments, R²³ is unsubstituted cycloalkyl. In some embodiments, R²³ is R²⁴-substituted heterocycloalkyl. In some embodiments, R²³ is unsubstituted heterocycloalkyl. In some embodiments, R²³ is R²⁴-substituted aryl. In some embodiments, R²³ is unsubstituted aryl. In some embodiments, R²³ is R²⁴-substituted heteroaryl. In some embodiments, R²³ is unsubstituted heteroaryl. In some embodiments, R²³ is substituted with one R²⁴. In some embodiments, R²³ is substituted with two optionally different R²⁴. In some embodiments, R²³ is substituted with three optionally different R²⁴. In some embodiments, R²³ is substituted with four optionally different R²⁴. In some embodiments, R²³ is independently oxo. In some embodiments, R²³ is independently —Br. In some embodiments, R²³ is independently —F. In some embodiments, R²³ is independently —Cl. In some embodiments, R²³ is independently —I. In some embodiments, R²³ is independently —CH₃. In some embodiments, R²³ is independently —OCH₃. In some embodiments, R²³ is independently (C₁-C₄) alkyl. In some embodiments, R²³ is independently (C₁-C₈) alkyl. In some embodiments, R²³ is independently (C₇-C₁₀) alkyl. In some embodiments, R²³ is independently (C₆-C₁₂) alkyl. In some embodiments, R²³ is independently phenyl. In some embodiments, R²³ is independently —OH. In some embodiments, R²³ is independently —CF₃.

In some embodiments, two adjacent R²⁴ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁴ substituents are joined to form a substituted cycloalkyl. In some embodiments, two adjacent R²⁴ substituents are joined to form an unsubstituted cycloalkyl. In some embodiments, two adjacent R²⁴ substituents are joined to form a substituted heterocycloalkyl. In some embodiments, two adjacent R²⁴ substituents are joined to form an unsubstituted heterocycloalkyl. In some embodiments, two adjacent R²⁴ substituents are joined to form a substituted aryl. In some embodiments, two adjacent R²⁴ substituents are joined to form an unsubstituted aryl. In some embodiments, two adjacent R²⁴ substituents are joined to form a substituted heteroaryl. In some embodiments, two adjacent R²⁴ substituents are joined to form an unsubstituted heteroaryl.

In some embodiments, R²⁴ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R²⁴ is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, or R²⁵-substituted or unsubstituted heteroaryl. In some embodiments, R²⁴ is CH₃. In some embodiments, R²⁴ is independently R²⁵-substituted or unsubstituted piperidinyl, R²⁵-substituted or unsubstituted piperazinyl, R²⁵-substituted or unsubstituted thiazolyl, R²⁵-substituted or unsubstituted oxazolyl, R²⁵-substituted or unsubstituted phenyl, R²⁵-substituted or unsubstituted thienyl, R²⁵-substituted or unsubstituted 4,5,6,7-tetrahydrobenzo[b]thienyl, R²⁵-substituted or unsubstituted pyridyl, or R²⁵-substituted or unsubstituted 1,3,4-thiadiazolyl. In some embodiments, R²⁴ is R²⁵-substituted or unsubstituted benzo[d]oxazolyl. In some embodiments, R²⁴ is R²⁵-substituted or unsubstituted imidazolyl. In some embodiments, R²⁴ is R²⁵-substituted imidazolyl. In some embodiments, R²⁴ is unsubstituted imidazolyl. In some embodiments, R²⁴ is R²⁵-substituted or unsubstituted 4H-1,2,4-triazolyl. In some embodiments, R²⁴ is R²⁵-substituted 4H-1,2,4-triazolyl. In some embodiments, R²⁴ is unsubstituted 4H-1,2,4-triazolyl. In some embodiments, R²⁴ is R²⁵-substituted or unsubstituted triazolyl. In some embodiments, R²⁴ is R²⁵-substituted triazolyl. In some embodiments, R²⁴ is unsubstituted triazolyl. In some embodiments, R²⁴ is independently R²⁵-substituted or unsubstituted alkyl, R²⁵-substituted or unsubstituted heteroalkyl, R²⁵-substituted or unsubstituted cycloalkyl, R²⁵ substituted or unsubstituted heterocycloalkyl, R²⁵-substituted or unsubstituted aryl, or R²⁵-substituted or unsubstituted heteroaryl. In some embodiments, R²⁴ is independently R²⁵-substituted alkyl. In some embodiments, R²⁴ is unsubstituted alkyl. In some embodiments, R²⁴ is R²⁵-substituted heteroalkyl. In some embodiments, R²⁴ is unsubstituted heteroalkyl. In some embodiments, R²⁴ is R²⁵-substituted substituted cycloalkyl. In some embodiments, R²⁴ is unsubstituted cycloalkyl. In some embodiments, R²⁴ is R²⁵-substituted heterocycloalkyl. In some embodiments, R²⁴ is unsubstituted heterocycloalkyl. In some embodiments, R²⁴ is R²⁵-substituted aryl. In some embodiments, R²⁴ is unsubstituted aryl. In some embodiments, R²⁴ is R²⁵-substituted heteroaryl. In some embodiments, R²⁴ is unsubstituted heteroaryl. In some embodiments, R²⁴ is substituted with one R²⁵. In some embodiments, R²⁴ is substituted with two optionally different R²⁵. In some embodiments, R²⁴ is substituted with three optionally different R²⁵. In some embodiments, R²⁴ is substituted with four optionally different R²⁵. In some embodiments, R²⁴ is independently oxo. In some embodiments, R²⁴ is independently —Br. In some embodiments, R²⁴ is independently —F. In some embodiments, R²⁴ is independently —Cl. In some embodiments, R²⁴ is independently —I. In some embodiments, R²⁴ is independently —CH₃. In some embodiments, R²⁴ is independently —OCH₃. In some embodiments, R²⁴ is independently (C₁-C₄) alkyl. In some embodiments, R²⁴ is independently (C₁-C₈) alkyl. In some embodiments, R²⁴ is independently (C₇-C₁₀) alkyl. In some embodiments, R²⁴ is independently (C₆-C₁₂) alkyl. In some embodiments, R²⁴ is independently phenyl. In some embodiments, R²⁴ is independently —OH. In some embodiments, R²⁴ is independently —CF₃. In some embodiments, R²⁴ is R²⁵-substituted or unsubstituted benzo[d]oxazolyl. In some embodiments, R²⁴ is R²⁵-substituted benzo[d]oxazolyl. In some embodiments, R²⁴ is unsubstituted benzo[d]oxazolyl. In some embodiments, R²⁴ is R²⁵-substituted or unsubstituted imidazolyl. In some embodiments, R²⁴ is R²⁵-substituted imidazolyl. In some embodiments, R²⁴ is unsubstituted imidazolyl. In some embodiments, R²⁴ is R²⁵-substituted or unsubstituted 4H-1,2,4-triazolyl. In some embodiments, R²⁴ is R²⁵-substituted 4H-1,2,4-triazolyl. In some embodiments, R²⁴ is unsubstituted 4H-1,2,4-triazolyl. In some embodiments, R²⁴ is R²⁵-substituted or unsubstituted triazolyl. In some embodiments, R²⁴ is R²⁵-substituted triazolyl. In some embodiments, R²⁴ is unsubstituted triazolyl. In some embodiments, R²⁴ is R²⁵-substituted or unsubstituted (C₁-C₄) alkyl. In some embodiments, R²⁴ is 2-propyl. In some embodiments, R²⁴ is methyl. In some embodiments, R²⁴ is ethyl. In some embodiments, R²⁴ is propyl. In some embodiments, R²⁴ is butyl. In some embodiments, R²⁴ is cyclopropyl. In some embodiments, R²⁴ is cyclobutyl.

In some embodiments, R³ is hydrogen or substituted or unsubstituted alkyl. In some embodiments, R³ is hydrogen. In some embodiments, R³ is substituted or unsubstituted alkyl. In some embodiments, R³ is substituted alkyl. In some embodiments, R³ is unsubstituted alkyl.

In some embodiments, R³ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R³ is substituted or unsubstituted alkyl. In some embodiments, R³ is substituted or unsubstituted heteroalkyl. In some embodiments, R³ is substituted or unsubstituted cycloalkyl. In some embodiments, R³ is substituted or unsubstituted heterocycloalkyl. In some embodiments, R³ is substituted or unsubstituted aryl. In some embodiments, R³ is substituted or unsubstituted heteroaryl. In some embodiments, R³ is unsubstituted alkyl. In some embodiments, R³ is unsubstituted heteroalkyl. In some embodiments, R³ is unsubstituted cycloalkyl. In some embodiments, R³ is unsubstituted heterocycloalkyl. In some embodiments, R³ is unsubstituted aryl. In some embodiments, R³ is unsubstituted heteroaryl. In some embodiments, R³ is substituted alkyl. In some embodiments, R³ is substituted heteroalkyl. In some embodiments, R³ is substituted cycloalkyl. In some embodiments, R³ is substituted heterocycloalkyl. In some embodiments, R³ is substituted aryl. In some embodiments, R³ is substituted heteroaryl. In some embodiments, R³ is R²⁶-substituted alkyl. In some embodiments, R³ is R²⁶-substituted heteroalkyl. In some embodiments, R³ is R²⁶-substituted cycloalkyl. In some embodiments, R³ is R²⁶-substituted heterocycloalkyl. In some embodiments, R³ is R²⁶-substituted aryl. In some embodiments, R³ is R²⁶-substituted heteroaryl. In some embodiments, R³ is substituted or unsubstituted phenyl. In some embodiments, R³ is R²⁶-substituted or unsubstituted phenyl. In some embodiments, R³ is substituted phenyl. In some embodiments, R³ is unsubstituted phenyl. In some embodiments, R³ is R²⁶-substituted phenyl. In some embodiments, R³ is substituted or unsubstituted thienyl. In some embodiments, R³ is R²⁶-substituted or unsubstituted thienyl. In some embodiments, R³ is substituted thienyl. In some embodiments, R³ is unsubstituted thienyl. In some embodiments, R³ is R²⁶-substituted thienyl. In some embodiments, R³ is substituted or unsubstituted 4,5,6,7-tetrahydrobenzo[b]thienyl. In some embodiments, R³ is R²⁶-substituted or unsubstituted 4,5,6,7-tetrahydrobenzo[b]thienyl. In some embodiments, R³ is substituted 4,5,6,7-tetrahydrobenzo[b]thienyl. In some embodiments, R³ is unsubstituted 4,5,6,7-tetrahydrobenzo[b]thienyl. In some embodiments, R³ is R²⁶-substituted 4,5,6,7-tetrahydrobenzo[b]thienyl. In some embodiments, R³ is substituted or unsubstituted pyridyl. In some embodiments, R³ is R²⁶-substituted or unsubstituted pyridyl. In some embodiments, R³ is substituted pyridyl. In some embodiments, R³ is unsubstituted pyridyl. In some embodiments, R³ is R²⁶-substituted pyridyl. In some embodiments, R³ is substituted or unsubstituted 1,3,4-thiadiazolyl. In some embodiments, R³ is R²⁶-substituted or unsubstituted 1,3,4-thiadiazolyl. In some embodiments, R³ is substituted 1,3,4-thiadiazolyl. In some embodiments, R³ is unsubstituted 1,3,4-thiadiazolyl. In some embodiments, R³ is R²⁶-substituted 1,3,4-thiadiazolyl. In some embodiments, R³ is substituted or unsubstituted piperidinyl. In some embodiments, R³ is R²⁶-substituted or unsubstituted piperidinyl. In some embodiments, R³ is substituted piperidinyl. In some embodiments, R³ is unsubstituted piperidinyl. In some embodiments, R³ is R²⁶-substituted piperidinyl. In some embodiments, R³ is substituted or unsubstituted piperazinyl. In some embodiments, R³ is R²⁶-substituted or unsubstituted piperazinyl. In some embodiments, R³ is substituted piperazinyl. In some embodiments, R³ is unsubstituted piperazinyl. In some embodiments, R³ is R²⁶-substituted piperazinyl. In some embodiments, R³ is substituted or unsubstituted oxazolyl. In some embodiments, R³ is R²⁶-substituted or unsubstituted oxazolyl. In some embodiments, R³ is substituted oxazolyl. In some embodiments, R³ is unsubstituted oxazolyl. In some embodiments, R³ is R²⁶-substituted oxazolyl. In some embodiments, R³ is substituted or unsubstituted thiazolyl. In some embodiments, R³ is R²⁶-substituted or unsubstituted thiazolyl. In some embodiments, R³ is substituted thiazolyl. In some embodiments, R³ is unsubstituted thiazolyl. In some embodiments, R³ is R²⁶-substituted thiazolyl. In some embodiments, R³ is R²⁶-substituted or unsubstituted benzo[d]oxazolyl. In some embodiments, R³ is R²⁶-substituted benzo[d]oxazolyl. In some embodiments, R³ is unsubstituted benzo[d]oxazolyl. In some embodiments, R³ is substituted or unsubstituted tetrahydrothienyl. In some embodiments, R³ is R²⁶-substituted or unsubstituted tetrahydrothienyl. In some embodiments, R³ is substituted tetrahydrothienyl. In some embodiments, R³ is unsubstituted tetrahydrothienyl. In some embodiments, R³ is R²⁶-substituted tetrahydrothienyl. In some embodiments, R³ is substituted or unsubstituted 2,3-dihydro-1H-pyrazolyl. In some embodiments, R³ is R²⁶-substituted or unsubstituted 2,3-dihydro-1H-pyrazolyl. In some embodiments, R³ is substituted 2,3-dihydro-1H-pyrazolyl. In some embodiments, R³ is unsubstituted 2,3-dihydro-1H-pyrazolyl. In some embodiments, R³ is R²⁶-substituted 2,3-dihydro-1H-pyrazolyl. In some embodiments, R³ is substituted or unsubstituted (C₁-C₄) alkyl. In some embodiments, R³ is R²⁶-substituted or unsubstituted (C₁-C₄) alkyl. In some embodiments, R³ is substituted (C₁-C₄) alkyl. In some embodiments, R³ is unsubstituted (C₁-C₄) alkyl. In some embodiments, R³ is R²⁶-substituted (C₁-C₄) alkyl. In some embodiments, R³ is substituted or unsubstituted ethyl. In some embodiments, R³ is R²⁶-substituted or unsubstituted ethyl. In some embodiments, R³ is substituted ethyl. In some embodiments, R³ is unsubstituted ethyl. In some embodiments, R³ is R²⁶-substituted ethyl.

In some embodiments, R³ is R²⁶-substituted or unsubstituted imidazolyl. In some embodiments, R³ is substituted imidazolyl. In some embodiments, R³ is unsubstituted imidazolyl. In some embodiments, R³ is R²⁶-substituted or unsubstituted 4H-1,2,4-triazolyl. In some embodiments, R³ is substituted 4H-1,2,4-triazolyl. In some embodiments, R³ is unsubstituted 4H-1,2,4-triazolyl. In some embodiments, R³ is R²⁶-substituted or unsubstituted triazolyl. In some embodiments, R³ is substituted triazolyl. In some embodiments, R³ is unsubstituted triazolyl. In some embodiments, R³ is substituted or unsubstituted naphthyl. In some embodiments, R³ is substituted or unsubstituted furanyl. In some embodiments, R³ is substituted or unsubstituted quinolinyl. In some embodiments, R³ is unsubstituted naphthyl. In some embodiments, R³ is unsubstituted furanyl. In some embodiments, R³ is unsubstituted quinolinyl. In some embodiments, R³ is substituted naphthyl. In some embodiments, R³ is substituted furanyl. In some embodiments, R³ is substituted quinolinyl. In some embodiments, R³ is R²⁶-substituted naphthyl. In some embodiments, R³ is R²⁶-substituted furanyl. In some embodiments, R³ is R²⁶-substituted quinolinyl. In some embodiments, R³ is substituted or unsubstituted morpholinyl. In some embodiments, R³ is R²⁶-substituted or unsubstituted morpholinyl. In some embodiments, R³ is substituted morpholinyl. In some embodiments, R³ is unsubstituted morpholinyl. In some embodiments, R³ is R²⁶-substituted morpholinyl. In some embodiments, R³ is substituted or unsubstituted piperazinyl. In some embodiments, R³ is R²⁶-substituted or unsubstituted piperazinyl. In some embodiments, R³ is substituted piperazinyl. In some embodiments, R³ is unsubstituted piperazinyl. In some embodiments, R³ is R²⁶-substituted piperazinyl. In some embodiments, R³ is substituted or unsubstituted pyrazolyl (e.g. 1H-pyrazolyl). In some embodiments, R³ is R²⁶-substituted or unsubstituted pyrazolyl (e.g. 1H-pyrazolyl). In some embodiments, R³ is substituted pyrazolyl (e.g. 1H-pyrazolyl). In some embodiments, R³ is unsubstituted pyrazolyl (e.g. 1H-pyrazolyl). In some embodiments, R³ is R²⁶-substituted pyrazolyl (e.g. 1H-pyrazolyl). In some embodiments, R³ is substituted or unsubstituted isoxazolyl. In some embodiments, R³ is R²⁶-substituted or unsubstituted isoxazolyl. In some embodiments, R³ is substituted isoxazolyl. In some embodiments, R³ is unsubstituted isoxazolyl. In some embodiments, R³ is R²⁶-substituted isoxazolyl.

In some embodiments, R³ is substituted with one R²⁶. In some embodiments, R³ is substituted with two optionally different R²⁶. In some embodiments, R³ is substituted with three optionally different R²⁶. In some embodiments, R³ is substituted with four optionally different R²⁶. In some embodiments, two adjacent R²⁶ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁶ substituents are joined to form a substituted cycloalkyl. In some embodiments, two adjacent R²⁶ substituents are joined to form an unsubstituted cycloalkyl. In some embodiments, two adjacent R²⁶ substituents are joined to form a substituted heterocycloalkyl. In some embodiments, two adjacent R²⁶ substituents are joined to form an unsubstituted heterocycloalkyl. In some embodiments, two adjacent R²⁶ substituents are joined to form a substituted aryl. In some embodiments, two adjacent R²⁶ substituents are joined to form an unsubstituted aryl. In some embodiments, two adjacent R²⁶ substituents are joined to form a substituted heteroaryl. In some embodiments, two adjacent R²⁶ substituents are joined to form an unsubstituted heteroaryl.

In some embodiments, R²⁶ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R²⁷-substituted or unsubstituted piperidinyl, R²⁷-substituted or unsubstituted piperazinyl, R²⁷-substituted or unsubstituted thiazolyl, R²⁷-substituted or unsubstituted oxazolyl, R²⁷-substituted or unsubstituted phenyl, R²⁷-substituted or unsubstituted thienyl, R²⁷-substituted or unsubstituted 4,5,6,7-tetrahydrobenzo[b]thienyl, R²⁷-substituted or unsubstituted pyridyl, or R²⁷-substituted or unsubstituted 1,3,4-thiadiazolyl. In some embodiments, R²⁶ is R²⁷⁻substituted or unsubstituted benzo[d]oxazolyl. In some embodiments, R²⁶ is R²⁷-substituted or unsubstituted imidazolyl. In some embodiments, R²⁶ is R²⁷-substituted imidazolyl. In some embodiments, R²⁶ is unsubstituted imidazolyl. In some embodiments, R²⁶ is R²⁷-substituted or unsubstituted 4H-1,2,4-triazolyl. In some embodiments, R²⁶ is R²⁷-substituted 4H-1,2,4-triazolyl. In some embodiments, R²⁶ is unsubstituted 4H-1,2,4-triazolyl. In some embodiments, R²⁶ is R²⁷-substituted substituted or unsubstituted triazolyl. In some embodiments, R²⁶ is R²⁷-substituted triazolyl. In some embodiments, R²⁶ is unsubstituted triazolyl. In some embodiments, R²⁶ is independently R²⁷-substituted or unsubstituted alkyl, R²⁷-substituted or unsubstituted heteroalkyl, R²⁷-substituted or unsubstituted cycloalkyl, R²⁷ substituted or unsubstituted heterocycloalkyl, R²⁷-substituted or unsubstituted aryl, or R²⁷-substituted or unsubstituted heteroaryl. In some embodiments, R²⁶ is independently R²⁷-substituted alkyl. In some embodiments, R²⁶ is unsubstituted alkyl. In some embodiments, R²⁶ is R²⁷-substituted heteroalkyl. In some embodiments, R²⁶ is unsubstituted heteroalkyl. In some embodiments, R²⁶ is R²⁷-substituted cycloalkyl. In some embodiments, R²⁶ is unsubstituted cycloalkyl. In some embodiments, R²⁶ is R²⁷-substituted heterocycloalkyl. In some embodiments, R²⁶ is unsubstituted heterocycloalkyl. In some embodiments, R²⁶ is R²⁷-substituted aryl. In some embodiments, R²⁶ is unsubstituted aryl. In some embodiments, R²⁶ is R²⁷-substituted heteroaryl. In some embodiments, R²⁶ is unsubstituted heteroaryl. In some embodiments, R²⁶ is substituted with one R²⁷. In some embodiments, R²⁶ is substituted with two optionally different R²⁷. In some embodiments, R²⁶ is substituted with three optionally different R²⁷. In some embodiments, R²⁶ is substituted with four optionally different R²⁷. In some embodiments, R²⁶ is independently oxo. In some embodiments, R²⁶ is independently —Br. In some embodiments, R²⁶ is independently —F. In some embodiments, R²⁶ is independently —Cl. In some embodiments, R²⁶ is independently —I. In some embodiments, R²⁶ is independently —CH₃. In some embodiments, R²⁶ is independently —OCH₃. In some embodiments, R²⁶ is independently (C₁-C₄) alkyl. In some embodiments, R²⁶ is independently (C₁-C₈) alkyl. In some embodiments, R²⁶ is independently (C₇-C₁₀) alkyl. In some embodiments, R²⁶ is independently (C₆-C₁₂) alkyl. In some embodiments, R²⁶ is independently phenyl. In some embodiments, R²⁶ is independently —OH. In some embodiments, R²⁶ is independently —CF₃. In some embodiments, R²⁶ is independently —CN. In some embodiments, R²⁶ is independently phenyl.

In some embodiments, two adjacent R²⁷ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁷ substituents are joined to form a substituted cycloalkyl. In some embodiments, two adjacent R²⁷ substituents are joined to form an unsubstituted cycloalkyl. In some embodiments, two adjacent R²⁷ substituents are joined to form a substituted heterocycloalkyl. In some embodiments, two adjacent R²⁷ substituents are joined to form an unsubstituted heterocycloalkyl. In some embodiments, two adjacent R²⁷ substituents are joined to form a substituted aryl. In some embodiments, two adjacent R²⁷ substituents are joined to form an unsubstituted aryl. In some embodiments, two adjacent R²⁷ substituents are joined to form a substituted heteroaryl. In some embodiments, two adjacent R²⁷ substituents are joined to form an unsubstituted heteroaryl.

In some embodiments, R²⁷ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R²⁷ is R²⁸-substituted or unsubstituted alkyl, R²⁸-substituted or unsubstituted heteroalkyl, R²⁸-substituted or unsubstituted cycloalkyl, R²⁸-substituted or unsubstituted heterocycloalkyl, R²⁸-substituted or unsubstituted aryl, or R²⁸-substituted or unsubstituted heteroaryl. In some embodiments, R²⁷ is CH₃. In some embodiments, R²⁷ is independently R²⁸-substituted or unsubstituted piperidinyl, R²⁸-substituted or unsubstituted piperazinyl, R²⁸-substituted or unsubstituted thiazolyl, R²⁸-substituted or unsubstituted oxazolyl, R²⁸-substituted or unsubstituted phenyl, R²⁸-substituted or unsubstituted thienyl, R²⁸-substituted or unsubstituted 4,5,6,7-tetrahydrobenzo[b]thienyl, R²⁸-substituted or unsubstituted pyridyl, or R²⁸-substituted or unsubstituted 1,3,4-thiadiazolyl. In some embodiments, R²⁷ is R²⁸-substituted or unsubstituted benzo[d]oxazolyl. In some embodiments, R²⁷ is R²⁸-substituted or unsubstituted imidazolyl. In some embodiments, R²⁷ is R²⁸-substituted imidazolyl. In some embodiments, R²⁷ is unsubstituted imidazolyl. In some embodiments, R²⁷ is R²⁸-substituted or unsubstituted 4H-1,2,4-triazolyl. In some embodiments, R²⁷ is R²⁸-substituted 4H-1,2,4-triazolyl. In some embodiments, R²⁷ is unsubstituted 4H-1,2,4-triazolyl. In some embodiments, R²⁷ is R²⁸-substituted or unsubstituted triazolyl. In some embodiments, R²⁷ is R²⁸-substituted triazolyl. In some embodiments, R²⁷ is unsubstituted triazolyl. In some embodiments, R²⁷ is independently R²⁸-substituted or unsubstituted alkyl, R²⁸-substituted or unsubstituted heteroalkyl, R²⁸-substituted or unsubstituted cycloalkyl, R²⁸ substituted or unsubstituted heterocycloalkyl, R²⁸-substituted or unsubstituted aryl, or R²⁸-substituted or unsubstituted heteroaryl. In some embodiments, R²⁷ is independently R²⁸-substituted alkyl. In some embodiments, R²⁷ is unsubstituted alkyl. In some embodiments, R²⁷ is R²⁸-substituted heteroalkyl. In some embodiments, R²⁷ is unsubstituted heteroalkyl. In some embodiments, R²⁷ is R²⁸-substituted cycloalkyl. In some embodiments, R²⁷ is unsubstituted cycloalkyl. In some embodiments, R²⁷ is R²⁸-substituted heterocycloalkyl. In some embodiments, R²⁷ is unsubstituted heterocycloalkyl. In some embodiments, R²⁷ is R²⁸-substituted aryl. In some embodiments, R²⁷ is unsubstituted aryl. In some embodiments, R²⁷ is R²⁸-substituted heteroaryl. In some embodiments, R²⁷ is unsubstituted heteroaryl. In some embodiments, R²⁷ is substituted with one R²⁸. In some embodiments, R²⁷ is substituted with two optionally different R²⁸. In some embodiments, R²⁷ is substituted with three optionally different R²⁸. In some embodiments, R²⁷ is substituted with four optionally different R²⁸. In some embodiments, R²⁷ is independently oxo. In some embodiments, R²⁷ is independently —Br. In some embodiments, R²⁷ is independently —F. In some embodiments, R²⁷ is independently —Cl. In some embodiments, R²⁷ is independently —I. In some embodiments, R²⁷ is independently —CH₃. In some embodiments, R²⁷ is independently —OCH₃. In some embodiments, R²⁷ is independently (C₁-C₄) alkyl. In some embodiments, R²⁷ is independently (C₁-C₈) alkyl. In some embodiments, R²⁷ is independently (C₇-C₁₀) alkyl. In some embodiments, R²⁷ is independently (C₆-C₁₂) alkyl. In some embodiments, R²⁷ is independently phenyl. In some embodiments, R²⁷ is independently —OH. In some embodiments, R²⁷ is independently —CF₃. In some embodiments, R²⁷ is R²⁸-substituted or unsubstituted benzo[d]oxazolyl. In some embodiments, R²⁷ is R²⁸-substituted benzo[d]oxazolyl. In some embodiments, R²⁷ is unsubstituted benzo[d]oxazolyl. In some embodiments, R²⁷ is R²⁸-substituted or unsubstituted imidazolyl. In some embodiments, R²⁷ is R²⁸-substituted imidazolyl. In some embodiments, R²⁷ is unsubstituted imidazolyl. In some embodiments, R²⁷ is R²⁸-substituted or unsubstituted 4H-1,2,4-triazolyl. In some embodiments, R²⁷ is R²⁸-substituted 4H-1,2,4-triazolyl. In some embodiments, R²⁷ is unsubstituted 4H-1,2,4-triazolyl. In some embodiments, R²⁷ is R²⁸-substituted or unsubstituted triazolyl. In some embodiments, R²⁷ is R²⁸-substituted triazolyl. In some embodiments, R²⁷ is unsubstituted triazolyl. In some embodiments, R²⁷ is R²⁸-substituted or unsubstituted (C₁-C₄) alkyl. In some embodiments, R²⁷ is 2-propyl. In some embodiments, R²⁷ is methyl. In some embodiments, R²⁷ is ethyl. In some embodiments, R²⁷ is propyl. In some embodiments, R²⁷ is butyl. In some embodiments, R²⁷ is cyclopropyl. In some embodiments, R²⁷ is cyclobutyl.

In some embodiments, R⁴ is hydrogen, substituted or unsubstituted (C₁-C₄)alkyl, substituted or unsubstituted (C₃-C₆)cycloalkyl, or aryl. In some embodiments, R⁴ is hydrogen. In some embodiments, R⁴ is methyl. In some embodiments, R⁴ is ethyl. In some embodiments, R⁴ is n-propyl. In some embodiments, R⁴ is isopropyl. In some embodiments, R⁴ is t-butyl. In some embodiments, R⁴ is hydrogen, substituted or unsubstituted (C₁-C₄)alkyl. In some embodiments, R⁴ is methyl, ethyl,n-propyl, isopropyl, t-butyl, or —CF₃. In some embodiments, R⁴ is substituted or unsubstituted (C₃-C₆)cycloalkyl. In some embodiments, R⁴ is substituted (C₃-C₆)cycloalkyl. In some embodiments, R⁴ is unsubstituted (C₃-C₆)cycloalkyl. In some embodiments, R⁴ is cyclopropyl, cyclobutyl, cyclopentyl, or cyclohexyl. In some embodiments, R⁴ is cyclopropyl. In some embodiments, R⁴ is cyclobutyl. In some embodiments, R⁴ is cyclopentyl. In some embodiments, R⁴ is cyclohexyl. In some embodiments, R⁴ is —C(O)R⁹. In some embodiments, R⁴ is —C(O)CH₃. In some embodiments, R⁴ is —C(O)CH₂CH₃. In some embodiments, R⁴ is —CH₂CCH.

In some embodiments, R⁷, R⁸, R⁹, and R¹⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹¹ is substituted or unsubstituted heteroaryl or —C(O)R¹². In some embodiments, R⁹ is CH₂CCH. In some embodiments, R⁹ is CH₃. In some embodiments, R¹⁹ is CH₂CH₃. In some embodiments, R¹⁹ is CH₃.

In some embodiments, R¹¹ is substituted or unsubstituted heteroaryl. In some embodiments, R¹¹ is substituted heteroaryl. In some embodiments, R¹¹ is substituted pyridinyl (pyridyl). In some embodiments, R¹¹ is unsubstituted heteroaryl. In some embodiments, R¹² is substituted or unsubstituted cycloalkyl. In some embodiments, R¹² is unsubstituted cycloalkyl. In some embodiments, R¹² is cyclopropyl. In some embodiments, R¹² is cyclobutyl. In some embodiments, R¹² is cyclopentyl.

In some embodiments, R¹¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹¹ is substituted or unsubstituted alkyl. In some embodiments, R¹¹ is substituted or unsubstituted heteroalkyl. In some embodiments, R¹¹ is substituted or unsubstituted cycloalkyl. In some embodiments, R¹¹ is substituted or unsubstituted heterocycloalkyl. In some embodiments, R¹¹ is substituted or unsubstituted aryl. In some embodiments, R¹¹ is substituted or unsubstituted heteroaryl. In some embodiments, R¹¹ is unsubstituted alkyl. In some embodiments, R¹¹ is unsubstituted heteroalkyl. In some embodiments, R¹¹ is unsubstituted cycloalkyl. In some embodiments, R¹¹ is unsubstituted heterocycloalkyl. In some embodiments, R¹¹ is unsubstituted aryl. In some embodiments, R¹¹ is unsubstituted heteroaryl. In some embodiments, R¹¹ is substituted alkyl. In some embodiments, R¹¹ is substituted heteroalkyl. In some embodiments, R¹¹ is substituted cycloalkyl. In some embodiments, R¹¹ is substituted heterocycloalkyl. In some embodiments, R¹¹ is substituted aryl. In some embodiments, R¹¹ is substituted heteroaryl. In some embodiments, R¹¹ is R⁵⁰-substituted alkyl. In some embodiments, R¹¹ is R⁵⁰-substituted heteroalkyl. In some embodiments, R¹¹ is R⁵⁰-substituted cycloalkyl. In some embodiments, R¹¹ is R⁵⁹-substituted heterocycloalkyl. In some embodiments, R¹¹ is R⁵⁰-substituted aryl. In some embodiments, R¹¹ is R⁵⁰-substituted heteroaryl. In some embodiments, R¹¹ is substituted or unsubstituted phenyl. In some embodiments, R¹¹ is R⁵⁰-substituted or unsubstituted phenyl. In some embodiments, R¹¹ is substituted phenyl. In some embodiments, R¹¹ is unsubstituted phenyl. In some embodiments, R¹¹ is R⁵⁰-substituted phenyl. In some embodiments, R¹¹ is substituted or unsubstituted thienyl. In some embodiments, R¹¹ is R⁵⁰-substituted or unsubstituted thienyl. In some embodiments, R¹¹ is substituted thienyl. In some embodiments, R¹¹ is unsubstituted thienyl. In some embodiments, R¹¹ is R⁵⁰-substituted thienyl. In some embodiments, R¹¹ is substituted or unsubstituted 4,5,6,7-tetrahydrobenzo[b]thienyl. In some embodiments, R¹¹ is R⁵⁰-substituted or unsubstituted 4,5,6,7-tetrahydrobenzo[b]thienyl. In some embodiments, R¹¹ is substituted 4,5,6,7-tetrahydrobenzo[b]thienyl. In some embodiments, R¹¹ is unsubstituted 4,5,6,7-tetrahydrobenzo[b]thienyl. In some embodiments, R¹¹ is R⁵⁰-substituted 4,5,6,7-tetrahydrobenzo[b]thienyl. In some embodiments, R¹¹ is substituted or unsubstituted pyridyl. In some embodiments, R¹¹ is R⁵⁰-substituted or unsubstituted pyridyl. In some embodiments, R¹¹ is substituted pyridyl. In some embodiments, R¹¹ is unsubstituted pyridyl. In some embodiments, R¹¹ is R⁵⁰-substituted pyridyl. In some embodiments, R¹¹ is substituted or unsubstituted 1,3,4-thiadiazolyl. In some embodiments, R¹¹ is R⁵⁰-substituted or unsubstituted 1,3,4-thiadiazolyl. In some embodiments, R¹¹ is substituted 1,3,4-thiadiazolyl. In some embodiments, R¹¹ is unsubstituted 1,3,4-thiadiazolyl. In some embodiments, R¹¹ is R⁵⁰-substituted 1,3,4-thiadiazolyl. In some embodiments, R¹¹ is substituted or unsubstituted piperidinyl. In some embodiments, R¹¹ is R⁵⁰-substituted or unsubstituted piperidinyl. In some embodiments, R¹¹ is substituted piperidinyl. In some embodiments, R¹¹ is unsubstituted piperidinyl. In some embodiments, R¹¹ is R⁵⁰-substituted piperidinyl. In some embodiments, R¹¹ is substituted or unsubstituted piperazinyl. In some embodiments, R¹¹ is R⁵⁰-substituted or unsubstituted piperazinyl. In some embodiments, R¹¹ is substituted piperazinyl. In some embodiments, R¹¹ is unsubstituted piperazinyl. In some embodiments, R¹¹ is R⁵⁰-substituted piperidinyl. In some embodiments, R¹¹ is substituted piperazinyl. In unsubstituted oxazolyl. In some embodiments, R¹¹ is R⁵⁰-substituted or unsubstituted oxazolyl. In some embodiments, R¹¹ is substituted oxazolyl. In some embodiments, R¹¹ is unsubstituted oxazolyl. In some embodiments, R¹¹ is R⁵⁰-substituted oxazolyl. In some embodiments, R¹¹ is substituted or unsubstituted thiazolyl. In some embodiments, R¹¹ is R⁵⁰-substituted or unsubstituted thiazolyl. In some embodiments, R¹¹ is substituted thiazolyl. In some embodiments, R¹¹ is unsubstituted thiazolyl. In some embodiments, R¹¹ is R⁵⁰-substituted thiazolyl. In some embodiments, R¹¹ is R⁵⁰-substituted embodiments, R¹¹ is substituted or unsubstituted benzo[d]oxazolyl. In some embodiments, R¹¹ is R⁵⁰-substituted benzo[d]oxazolyl. In some embodiments, R¹¹ is unsubstituted benzo[d]oxazolyl. In some embodiments, R¹¹ is substituted or unsubstituted tetrahydrothienyl. In some embodiments, R¹¹ is R⁵⁰-substituted or unsubstituted tetrahydrothienyl. In some embodiments, R¹¹ is substituted tetrahydrothienyl. In some embodiments, R¹¹ is unsubstituted tetrahydrothienyl. In some embodiments, R¹¹ is R⁵⁰-substituted tetrahydrothienyl. In some embodiments, R¹¹ is substituted or unsubstituted 2,3-dihydro-1H-pyrazolyl. In some embodiments, R¹¹ is R⁵⁰-substituted or unsubstituted 2,3-dihydro-1H-pyrazolyl. In some embodiments, R¹¹ is substituted 2,3-dihydro-1H-pyrazolyl. In some embodiments, R¹¹ is unsubstituted 2,3-dihydro-1H-pyrazolyl. In some embodiments, R¹¹ is R⁵⁰-substituted substituted 2,3-dihydro-1H-pyrazolyl. In some embodiments, R¹¹ is substituted or unsubstituted (C₁-C₄) alkyl. In some embodiments, R¹¹ is R⁵⁰-substituted or unsubstituted (C₁-C₄) alkyl. In some embodiments, R¹¹ is substituted (C₁-C₄) alkyl. In some embodiments, R¹¹ is unsubstituted (C₁-C₄) alkyl. In some embodiments, R¹¹ is R⁵⁰-substituted (C₁-C₄) alkyl. In some embodiments, R¹¹ is substituted or unsubstituted ethyl. In some embodiments, R¹¹ is R⁵⁰-substituted substituted or unsubstituted ethyl. In some embodiments, R¹¹ is substituted ethyl. In some embodiments, R¹¹ is unsubstituted ethyl. In some embodiments, R¹¹ is R⁵⁰-substituted ethyl.

In some embodiments, R¹¹ is R⁵⁰-substituted or unsubstituted imidazolyl. In some embodiments, R¹¹ is substituted imidazolyl. In some embodiments, R¹¹ is unsubstituted imidazolyl. In some embodiments, R¹¹ is R⁵⁰-substituted or unsubstituted 4H-1,2,4-triazolyl. In some embodiments, R¹¹ is substituted 4H-1,2,4-triazolyl. In some embodiments, R¹¹ is unsubstituted 4H-1,2,4-triazolyl. In some embodiments, R¹¹ is R⁵⁰-substituted or unsubstituted triazolyl. In some embodiments, R¹¹ is substituted triazolyl. In some embodiments, R¹¹ is unsubstituted triazolyl. In some embodiments, R¹¹ is substituted or unsubstituted naphthyl. In some embodiments, R¹¹ is substituted or unsubstituted furanyl. In some embodiments, R¹¹ is substituted or unsubstituted quinolinyl. In some embodiments, R¹¹ is unsubstituted naphthyl. In some embodiments, R¹¹ is unsubstituted furanyl. In some embodiments, R¹¹ is unsubstituted quinolinyl. In some embodiments, R¹¹ is substituted naphthyl. In some embodiments, R¹¹ is substituted furanyl. In some embodiments, R¹¹ is substituted quinolinyl. In some embodiments, R¹¹ is R⁵⁰-substituted substituted furanyl. In some embodiments, R¹¹ is R⁵⁰— R⁵⁰-substituted naphthyl. In some embodiments, R¹¹ is substituted quinolinyl. In some embodiments, R¹¹ is substituted or unsubstituted morpholinyl. In some embodiments, R¹¹ is R⁵⁰-substituted or unsubstituted morpholinyl. In some embodiments, R¹¹ is substituted morpholinyl. In some embodiments, R¹¹ is unsubstituted morpholinyl. In some embodiments, R¹¹ is R⁵⁰-substituted morpholinyl. In some embodiments, R¹¹ is substituted or unsubstituted piperazinyl. In some embodiments, R¹¹ is R⁵⁰-substituted or unsubstituted piperazinyl. In some embodiments, R¹¹ is substituted piperazinyl. In some embodiments, R¹¹ is unsubstituted embodiments, R¹¹ is R⁵⁰-substituted piperazinyl. In some embodiments, R¹¹ is substituted piperazinyl. In some embodiments, R¹¹ is substituted or unsubstituted pyrazolyl (e.g. 1H-pyrazolyl). In some embodiments, R¹¹ is R⁵⁰-substituted or unsubstituted pyrazolyl (e.g. 1H-pyrazolyl). In some embodiments, R¹¹ is substituted pyrazolyl (e.g. 1H-pyrazolyl). In some embodiments, R¹¹ is unsubstituted pyrazolyl (e.g. 1H-pyrazolyl). In some embodiments, R¹¹ is R⁵⁰-substituted pyrazolyl (e.g. 1H-pyrazolyl). In some embodiments, R¹¹ is substituted or unsubstituted isoxazolyl. In some embodiments, R¹¹ is R⁵⁰-substituted or unsubstituted isoxazolyl. In some embodiments, R¹¹ is substituted isoxazolyl. In some embodiments, R¹¹ is unsubstituted isoxazolyl. In some embodiments, R¹¹ is R⁵⁰-substituted isoxazolyl. In some embodiments, R¹¹ is substituted or unsubstituted isoquinolinyl. In some embodiments, R¹¹ is R⁵⁰-substituted or unsubstituted isoquinolinyl. In some embodiments, R¹¹ is substituted isoquinolinyl. In some embodiments, R¹¹ is unsubstituted isoquinolinyl. In some embodiments, R¹¹ is R⁵⁰-substituted isoquinolinyl. In some embodiments, R¹¹ is substituted or unsubstituted quinolinyl. In some embodiments, R¹¹ is R⁵⁰-substituted or unsubstituted quinolinyl. In some embodiments, R¹¹ is substituted quinolinyl. In some embodiments, R¹¹ is unsubstituted quinolinyl. In some embodiments, R¹¹ is R⁵⁰-substituted quinolinyl. In some embodiments, R¹¹ is substituted or unsubstituted pyrazinyl. In some embodiments, R¹¹ is R⁵⁰-substituted or unsubstituted pyrazinyl. In some embodiments, R¹¹ is substituted pyrazinyl. In some embodiments, R¹¹ is unsubstituted pyrazinyl. In some embodiments, R¹¹ is R⁵⁰-substituted pyrazinyl. In some embodiments, R⁵⁰ is independently —F, —OCH(CH₃)₂, —OCH₂CH₂OCH₃, —OCH₃, —N(CH₃)₂, methyl, —OCH₂CN, pyridinyl (pyridyl), piperazinyl, or 1-(4-methyl)piperazinyl. In some embodiments, R⁵⁰ is unsubstituted heteroaryl. In some embodiments, R⁵⁰ is pyridinyl (pyridyl). In some embodiments, R⁵⁰ is 2-pyridinyl (2-pyridyl). In some embodiments, R⁵⁰ is 3-pyridinyl (3-pyridyl). In some embodiments, R⁵⁰ is 4-pyridyl. In some embodiments, R⁵⁰ is substituted or unsubstituted aryl. In some embodiments, R¹ is unsubstituted aryl. In some embodiments, R⁵⁰ is phenyl. In some embodiments, R⁵⁰ is R⁵¹-substituted aryl. In some embodiments, R⁵⁰ is independently substituted with F, —OCH(CH₃)₂, —OCH₂CH₂OCH₃, —OCH₃, —N(CH₃)₂, methyl, —OCH₂CN, pyridyl, piperazinyl, or 1-(4-methyl)piperazinyl. In some embodiments, R⁵⁰ is cyclopropyl. In some embodiments, R⁵⁰ is —CF₃. In some embodiments, R⁵⁰ is —CH₂OH.

In some embodiments, R¹¹ is substituted with one R⁵⁰. In some embodiments, R¹¹ is substituted with two optionally different R⁵⁰. In some embodiments, R¹¹ is substituted with three optionally different R⁵⁰. In some embodiments, R¹¹ is substituted with four optionally different R⁵⁰. In some embodiments, two adjacent R⁵⁰ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R⁵⁰ substituents are joined to form a substituted cycloalkyl. In some embodiments, two adjacent R⁵⁰ substituents are joined to form an unsubstituted cycloalkyl. In some embodiments, two adjacent R⁵⁰ substituents are joined to form a substituted heterocycloalkyl. In some embodiments, two adjacent R⁵⁰ substituents are joined to form an unsubstituted heterocycloalkyl. In some embodiments, two adjacent R⁵⁰ substituents are joined to form a substituted aryl. In some embodiments, two adjacent R⁵⁰ substituents are joined to form an unsubstituted aryl. In some embodiments, two adjacent R⁵⁰ substituents are joined to form a substituted heteroaryl. In some embodiments, two adjacent R⁵⁰ substituents are joined to form an unsubstituted heteroaryl.

In some embodiments, R⁵⁰ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R⁵¹-substituted or unsubstituted piperidinyl, R⁵¹-substituted or unsubstituted piperazinyl, R⁵¹-substituted or unsubstituted thiazolyl, R⁵¹-substituted or unsubstituted oxazolyl, R⁵¹-substituted or unsubstituted phenyl, R⁵¹-substituted or unsubstituted thienyl, R⁵¹-substituted or unsubstituted 4,5,6,7-tetrahydrobenzo[b]thienyl, R⁵¹-substituted or unsubstituted pyridyl, or R⁵¹-substituted or unsubstituted 1,3,4-thiadiazolyl. In some embodiments, R⁵⁰ is R⁵¹-substituted or unsubstituted benzo[d]oxazolyl. In some embodiments, R⁵⁰ is R⁵¹-substituted or unsubstituted imidazolyl. In some embodiments, R⁵⁰ is R⁵¹-substituted imidazolyl. In some embodiments, R⁵⁰ is unsubstituted imidazolyl. In some embodiments, R⁵⁰ is R⁵¹-substituted or unsubstituted 4H-1,2,4-triazolyl. In some embodiments, R⁵⁰ is R⁵¹-substituted 4H-1,2,4-triazolyl. In some embodiments, R⁵⁰ is unsubstituted 4H-1,2,4-triazolyl. In some embodiments, R⁵⁰ is R⁵¹-substituted or unsubstituted triazolyl. In some embodiments, R⁵⁰ is R⁵¹-substituted triazolyl. In some embodiments, R⁵⁰ is unsubstituted triazolyl. In some embodiments, R⁵⁰ is independently R⁵¹-substituted or unsubstituted alkyl, R⁵¹-substituted or unsubstituted heteroalkyl, R⁵¹-substituted or unsubstituted cycloalkyl, R⁵¹ substituted or unsubstituted heterocycloalkyl, R⁵¹-substituted or unsubstituted aryl, or R⁵¹-substituted or unsubstituted heteroaryl. In some embodiments, R⁵⁰ is independently R⁵¹-substituted alkyl. In some embodiments, R⁵⁰ is unsubstituted alkyl. In some embodiments, R⁵⁰ is R⁵¹-substituted heteroalkyl. In some embodiments, R⁵⁰ is unsubstituted heteroalkyl. In some embodiments, R⁵⁰ is R⁵¹-substituted cycloalkyl. In some embodiments, R⁵⁰ is unsubstituted cycloalkyl. In some embodiments, R⁵⁰ is R⁵¹-substituted heterocycloalkyl. In some embodiments, R⁵⁰ is unsubstituted heterocycloalkyl. In some embodiments, R⁵⁰ is R⁵¹-substituted aryl. In some embodiments, R⁵⁰ is unsubstituted aryl. In some embodiments, R⁵⁰ is R⁵¹-substituted heteroaryl. In some embodiments, R⁵⁰ is unsubstituted heteroaryl. In some embodiments, R⁵⁰ is substituted with one R⁵¹. In some embodiments, R⁵⁰ is substituted with two optionally different R⁵¹. In some embodiments, R⁵⁰ is substituted with three optionally different R⁵¹. In some embodiments, R⁵⁰ is substituted with four optionally different R⁵¹. In some embodiments, R⁵⁰ is independently oxo. In some embodiments, R⁵⁰ is independently —Br. In some embodiments, R⁵⁰ is independently —F. In some embodiments, R⁵⁰ is independently —Cl. In some embodiments, R⁵⁰ is independently —I. In some embodiments, R⁵⁰ is independently —CH₃. In some embodiments, R⁵⁰ is independently —OCH₃. In some embodiments, R⁵⁰ is independently (C₁-C₄) alkyl. In some embodiments, R⁵⁰ is independently (C₁-C₈) alkyl. In some embodiments, R⁵⁰ is independently (C₇-C₁₀) alkyl. In some embodiments, R⁵⁰ is independently (C₆-C₁₂) alkyl. In some embodiments, R⁵⁰ is independently phenyl. In some embodiments, R⁵⁰ is independently —OH. In some embodiments, R⁵⁰ is independently —CF₃.

In some embodiments, two adjacent R⁵¹ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R⁵¹ substituents are joined to form a substituted cycloalkyl. In some embodiments, two adjacent R⁵¹ substituents are joined to form an unsubstituted cycloalkyl. In some embodiments, two adjacent R⁵¹ substituents are joined to form a substituted heterocycloalkyl. In some embodiments, two adjacent R⁵¹ substituents are joined to form an unsubstituted heterocycloalkyl. In some embodiments, two adjacent R⁵¹ substituents are joined to form a substituted aryl. In some embodiments, two adjacent R⁵¹ substituents are joined to form an unsubstituted aryl. In some embodiments, two adjacent R⁵¹ substituents are joined to form a substituted heteroaryl. In some embodiments, two adjacent R⁵¹ substituents are joined to form an unsubstituted heteroaryl.

In some embodiments, R⁵¹ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R⁵¹ is R⁵²-substituted or unsubstituted alkyl, R⁵²-substituted or unsubstituted heteroalkyl, R⁵²-substituted or unsubstituted cycloalkyl, R⁵²-substituted or unsubstituted heterocycloalkyl, R⁵²-substituted or unsubstituted aryl, or R⁵²-substituted or unsubstituted heteroaryl. In some embodiments, R⁵¹ is CH₃. In some embodiments, R⁵¹ is independently R⁵²-substituted or unsubstituted piperidinyl, R⁵²-substituted or unsubstituted piperazinyl, R⁵²-substituted or unsubstituted thiazolyl, R⁵²-substituted or unsubstituted oxazolyl, R⁵²-substituted or unsubstituted phenyl, R⁵²-substituted or unsubstituted thienyl, R⁵²-substituted or unsubstituted 4,5,6,7-tetrahydrobenzo[b]thienyl, R⁵²-substituted or unsubstituted pyridyl, or R⁵²-substituted or unsubstituted 1,3,4-thiadiazolyl. In some embodiments, R⁵¹ is R⁵²-substituted or unsubstituted benzo[d]oxazolyl. In some embodiments, R⁵¹ is R⁵²-substituted or unsubstituted imidazolyl. In some embodiments, R⁵¹ is R⁵²-substituted imidazolyl. In some embodiments, R⁵¹ is unsubstituted imidazolyl. In some embodiments, R⁵¹ is R⁵²-substituted or unsubstituted 4H-1,2,4-triazolyl. In some embodiments, R⁵¹ is R⁵²-substituted 4H-1,2,4-triazolyl. In some embodiments, R⁵¹ is unsubstituted 4H-1,2,4-triazolyl. In some embodiments, R⁵¹ is R⁵²-substituted or unsubstituted triazolyl. In some embodiments, R⁵¹ is R⁵²-substituted triazolyl. In some embodiments, R⁵¹ is unsubstituted triazolyl. In some embodiments, R⁵¹ is independently R⁵²-substituted or unsubstituted alkyl, R⁵²-substituted or unsubstituted heteroalkyl, R⁵²-substituted or unsubstituted cycloalkyl, R⁵² substituted or unsubstituted heterocycloalkyl, R⁵²-substituted or unsubstituted aryl, or R⁵²-substituted or unsubstituted heteroaryl. In some embodiments, R⁵¹ is independently R⁵²-substituted alkyl. In some embodiments, R⁵¹ is unsubstituted alkyl. In some embodiments, R⁵¹ is R⁵²-substituted heteroalkyl. In some embodiments, R⁵¹ is unsubstituted heteroalkyl. In some embodiments, R⁵¹ is R⁵²-substituted cycloalkyl. In some embodiments, R⁵¹ is unsubstituted cycloalkyl. In some embodiments, R⁵¹ is R⁵²-substituted heterocycloalkyl. In some embodiments, R⁵¹ is unsubstituted heterocycloalkyl. In some embodiments, R⁵¹ is R⁵²-substituted aryl. In some embodiments, R⁵¹ is unsubstituted aryl. In some embodiments, R⁵¹ is R⁵²-substituted heteroaryl. In some embodiments, R⁵¹ is unsubstituted heteroaryl. In some embodiments, R⁵¹ is substituted with one R⁵². In some embodiments, R⁵¹ is substituted with two optionally different R⁵². In some embodiments, R⁵¹ is substituted with three optionally different R⁵². In some embodiments, R⁵¹ is substituted with four optionally different R⁵². In some embodiments, R⁵¹ is independently oxo. In some embodiments, R⁵¹ is independently —Br. In some embodiments, R⁵¹ is independently —F. In some embodiments, R⁵¹ is independently —Cl. In some embodiments, R⁵¹ is independently —I. In some embodiments, R⁵¹ is independently —CH₃. In some embodiments, R⁵¹ is independently —OCH₃. In some embodiments, R⁵¹ is independently (C₁-C₄) alkyl. In some embodiments, R⁵¹ is independently (C₁-C₈) alkyl. In some embodiments, R⁵¹ is independently (C₇-C₁₀) alkyl. In some embodiments, R⁵¹ is independently (C₆-C₁₂) alkyl. In some embodiments, R⁵¹ is independently phenyl. In some embodiments, R⁵¹ is independently —OH. In some embodiments, R⁵¹ is independently —CF₃. In some embodiments, R⁵¹ is R⁵²-substituted or unsubstituted benzo[d]oxazolyl. In some embodiments, R⁵¹ is R⁵²-substituted benzo[d]oxazolyl. In some embodiments, R⁵¹ is unsubstituted benzo[d]oxazolyl. In some embodiments, R⁵¹ is R⁵²-substituted or unsubstituted imidazolyl. In some embodiments, R⁵¹ is R⁵²-substituted imidazolyl. In some embodiments, R⁵¹ is unsubstituted imidazolyl. In some embodiments, R⁵¹ is R⁵²-substituted or unsubstituted 4H-1,2,4-triazolyl. In some embodiments, R⁵¹ is R⁵²-substituted 4H-1,2,4-triazolyl. In some embodiments, R⁵¹ is unsubstituted 4H-1,2,4-triazolyl. In some embodiments, R⁵¹ is R⁵²-substituted or unsubstituted triazolyl. In some embodiments, R⁵¹ is R⁵²-substituted triazolyl. In some embodiments, R⁵¹ is unsubstituted triazolyl. In some embodiments, R⁵¹ is R⁵²-substituted or unsubstituted (C₁-C₄) alkyl. In some embodiments, R⁵¹ is 2-propyl. In some embodiments, R⁵¹ is methyl. In some embodiments, R⁵¹ is ethyl. In some embodiments, R⁵¹ is propyl. In some embodiments, R⁵¹ is butyl. In some embodiments, R⁵¹ is cyclopropyl. In some embodiments, R⁵¹ is cyclobutyl.

In some embodiments v is 1. In other embodiments, v is 2. In some embodiments m is 1. In some embodiments m is 2. In some embodiments n is 0. In some embodiments n is 1. In some embodiments n is 2. In some embodiments n is 3. In some embodiments n is 4. In some embodiments, X is —Cl. In some embodiments, X is —Br. In some embodiments, X is —I. In some embodiments, X is —F.

In some embodiments, the compound having formula I is a compound having the formula:

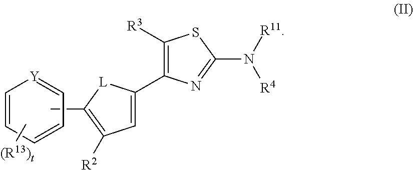

L, X, v, m, n, R¹, R², R³, R⁴, R⁶, R⁷, R⁸, R⁹, R¹⁰, R¹¹, and R¹² are as described herein (e.g. formula I, including embodiments).

R¹³ is independently hydrogen, halogen, —CX^{a}₃, —CN, —SO₂Cl, —SOᵣR¹⁷, —SOₚNR¹⁴R¹⁵, —NHNH₂, ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments R¹³ is attached to the ring at a position para or meta to the L-containing ring (i.e. the ring having an R² substituent). In some embodiments R¹³ is attached to the ring at a position para to the L-containing ring. In some embodiments R¹³ is attached to the ring at a position meta to the L-containing ring. In some embodiments, R¹³ is independently —F, —OCH(CH₃)₂, —OCH₂CH₂OCH₃, —OCH₃, —N(CH₃)₂, methyl, —OCH₂CN, pyridinyl (pyridyl), piperazinyl, or 1-(4-methyl)piperazinyl. X^{a} is independently —Cl, —Br, —I, or —F. In some embodiments, X^{a} is —Cl. In some embodiments, X^{a} is —Br. In some embodiments, X^{a} is —I. In some embodiments, X^{a} is —F.

In some embodiments, a compound of formula (II) is substituted with one R¹³. In some embodiments, a compound of formula (II) is substituted with two optionally different R¹³. In some embodiments, a compound of formula (II) is substituted with three optionally different R¹³. In some embodiments, a compound of formula (II) is substituted with four optionally different R¹³. In some embodiments, a compound of formula (II) is substituted with five optionally different R¹³. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents are joined to form a substituted cycloalkyl. In some embodiments, two adjacent R¹³ substituents are joined to form an unsubstituted cycloalkyl. In some embodiments, two adjacent R¹³ substituents are joined to form a substituted heterocycloalkyl. In some embodiments, two adjacent R¹³ substituents are joined to form an unsubstituted heterocycloalkyl. In some embodiments, two adjacent R¹³ substituents are joined to form a substituted aryl. In some embodiments, two adjacent R¹³ substituents are joined to form an unsubstituted aryl. In some embodiments, two adjacent R¹³ substituents are joined to form a substituted heteroaryl. In some embodiments, two adjacent R¹³ substituents are joined to form an unsubstituted heteroaryl.

In some embodiments, R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R¹³ is independently halogen, —CX^{a}₃, —CN, —N(O)_{q}, —NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹³ is independently halogen, —CF₃, —CN, —N(O)₂, —NH₂, or —OH. In some embodiments, R¹³ is independently unsubstituted alkyl, unsubstituted heteroalkyl, unsubstituted cycloalkyl, unsubstituted heterocycloalkyl, unsubstituted aryl, or unsubstituted heteroaryl. In some embodiments, R¹³ is —Cl. In some embodiments, R¹³ is —Br. In some embodiments, R¹³ is —I. In some embodiments, R¹³ is —F. In some embodiments, R¹³ is independently two different halogens. In some embodiments, R¹³ is —CF₃. In some embodiments, R¹³ is —CN. In some embodiments, R¹³ is —N(O)₂. In some embodiments, R¹³ is —NH₂. In some embodiments, R¹³ is —OH. In some embodiments, R¹³ is —C(O)CH₃. In some embodiments, R¹³ is —OCH₃. In some embodiments, R¹³ is unsubstituted phenyl.

In some embodiments, R¹³ is R⁵⁶-substituted or unsubstituted heteroaryl. In some embodiments, R¹³ is R⁵⁶-substituted heteroaryl. In some embodiments, R¹³ is unsubstituted heteroaryl. In some embodiments, R¹³ is R⁵⁶-substituted or unsubstituted piperidinyl. In some embodiments, R¹³ is R⁵⁶-substituted or unsubstituted piperazinyl. In some embodiments, R¹³ is R⁵⁶-substituted or unsubstituted thiazolyl. In some embodiments, R¹³ is R⁵⁶-substituted or unsubstituted oxazolyl. In some embodiments, R¹³ is R⁵⁶-substituted or unsubstituted phenyl. In some embodiments, R¹³ is R⁵⁶-substituted or unsubstituted thienyl. In some embodiments, R¹³ is R⁵⁶-substituted or unsubstituted 4,5,6,7-tetrahydrobenzo[b]thienyl. In some embodiments, R¹³ is R⁵⁶-substituted or unsubstituted pyridyl. In some embodiments, R¹³ is R⁵⁶-substituted or unsubstituted 1,3,4-thiadiazolyl. In some embodiments, R¹³ is R⁵⁶-substituted or unsubstituted benzo[d]oxazolyl. In some embodiments, R¹³ is R⁵⁶-substituted or unsubstituted imidazolyl. In some embodiments, R¹³ is R⁵⁶-substituted imidazolyl. In some embodiments, R¹³ is unsubstituted imidazolyl. In some embodiments, R¹³ is R⁵⁶-substituted or unsubstituted 4H-1,2,4-triazolyl. In some embodiments, R¹³ is R⁵⁶-substituted 4H-1,2,4-triazolyl. In some embodiments, R¹³ is unsubstituted 4H-1,2,4-triazolyl. In some embodiments, R¹³ is R⁵⁶-substituted or unsubstituted triazolyl. In some embodiments, R¹³ is R⁵⁶-substituted triazolyl. In some embodiments, R¹³ is unsubstituted triazolyl.

In some embodiments, R⁵⁶ is independently halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R⁵⁶ is independently R⁵⁷-substituted or unsubstituted alkyl, R⁵⁷-substituted or unsubstituted heteroalkyl, R⁵⁷-substituted or unsubstituted cycloalkyl, R⁵⁷ substituted or unsubstituted heterocycloalkyl, R⁵⁷-substituted or unsubstituted aryl, or R⁵⁷-substituted or unsubstituted heteroaryl. In some embodiments, R⁵⁶ is —CH₃.

In some embodiments, R¹³ is substituted with one R⁵⁶. In some embodiments, R¹³ is substituted with two optionally different R⁵⁶. In some embodiments, R¹³ is substituted with three optionally different R⁵⁶. In some embodiments, R¹³ is substituted with four optionally different R⁵⁶. In some embodiments, R¹³ is substituted with five optionally different R⁵⁶. In some embodiments, two adjacent R⁵⁶ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R⁵⁶ substituents are joined to form a substituted cycloalkyl. In some embodiments, two adjacent R⁵⁶ substituents are joined to form an unsubstituted cycloalkyl. In some embodiments, two adjacent R⁵⁶ substituents are joined to form a substituted heterocycloalkyl. In some embodiments, two adjacent R⁵⁶ substituents are joined to form an unsubstituted heterocycloalkyl. In some embodiments, two adjacent R⁵⁶ substituents are joined to form a substituted aryl. In some embodiments, two adjacent R⁵⁶ substituents are joined to form an unsubstituted aryl. In some embodiments, two adjacent R⁵⁶ substituents are joined to form a substituted heteroaryl. In some embodiments, two adjacent R⁵⁶ substituents are joined to form an unsubstituted heteroaryl.

In some embodiments, R¹³ is —NR¹⁴R¹⁵. In some embodiments, R¹⁵ is R⁶²-substituted or unsubstituted heteroaryl. In some embodiments, R¹⁵ is R⁶²-substituted heteroaryl. In some embodiments, R¹⁵ is unsubstituted heteroaryl. In some embodiments, R¹⁵ is R⁶²-substituted or unsubstituted piperidinyl, R⁶²-substituted or unsubstituted piperazinyl, R⁶²-substituted or unsubstituted thiazolyl, R⁶²-substituted or unsubstituted oxazolyl, R⁶²-substituted or unsubstituted phenyl, R⁶²-substituted or unsubstituted thienyl, R⁶²-substituted or unsubstituted 4,5,6,7-tetrahydrobenzo[b]thienyl, R⁶²-substituted or unsubstituted pyridyl, or R⁶²-substituted or unsubstituted 1,3,4-thiadiazolyl. In some embodiments, R¹⁵ is R⁶²-substituted or unsubstituted benzo[d]oxazolyl. In some embodiments, R¹⁵ is R⁶²-substituted or unsubstituted imidazolyl. In some embodiments, R¹⁵ is R⁶²-substituted imidazolyl. In some embodiments, R¹⁵ is unsubstituted imidazolyl. In some embodiments, R¹⁵ is R⁶²-substituted or unsubstituted 4H-1,2,4-triazolyl. In some embodiments, R¹⁵ is R⁶²-substituted 4H-1,2,4-triazolyl. In some embodiments, R¹⁵ is unsubstituted 4H-1,2,4-triazolyl. In some embodiments, R¹⁵ is R⁶²-substituted or unsubstituted triazolyl. In some embodiments, R¹⁵ is R⁶²-substituted triazolyl. In some embodiments, R¹⁵ is unsubstituted triazolyl. In some embodiments, R¹⁵ is R⁶²-substituted or unsubstituted benzo[g]quinolone. In some embodiments, R¹⁴ is R⁵⁹-substituted or unsubstituted benzo[g]quinolone. In some embodiments, R¹⁴ is R⁵⁹-substituted or unsubstituted heteroaryl. In some embodiments, R¹⁴ is R⁵⁹-substituted heteroaryl. In some embodiments, R¹⁴ is unsubstituted heteroaryl. In some embodiments, R¹⁴ is R⁵⁹-substituted or unsubstituted piperidinyl, R⁵⁹-substituted or unsubstituted piperazinyl, R⁵⁹-substituted or unsubstituted thiazolyl, R⁵⁹-substituted or unsubstituted oxazolyl, R⁵⁹-substituted or unsubstituted phenyl, R⁵⁹-substituted or unsubstituted thienyl, R⁵⁹-substituted or unsubstituted 4,5,6,7-tetrahydrobenzo[b]thienyl, R⁵⁹-substituted or unsubstituted pyridyl, or R⁵⁹-substituted or unsubstituted 1,3,4-thiadiazolyl. In some embodiments, R¹⁴ is R⁵⁹-substituted or unsubstituted benzo[d]oxazolyl. In some embodiments, R¹⁴ is R⁵⁹-substituted or unsubstituted imidazolyl. In some embodiments, R¹⁴ is R⁵⁹-substituted imidazolyl. In some embodiments, R¹⁴ is unsubstituted imidazolyl. In some embodiments, R¹⁴ is R⁵⁹-substituted or unsubstituted 4H-1,2,4-triazolyl. In some embodiments, R¹⁴ is R⁵⁹-substituted 4H-1,2,4-triazolyl. In some embodiments, R¹⁴ is unsubstituted 4H-1,2,4-triazolyl. In some embodiments, R¹⁴ is R⁵⁹-substituted or unsubstituted triazolyl. In some embodiments, R¹⁴ is R⁵⁹-substituted triazolyl. In some embodiments, R¹⁴ is unsubstituted triazolyl.

In some embodiments, the symbols p and q are independently an integer from 1 to 2. In some embodiments, the symbol r is independently an integer from 0 to 4. In some embodiments, the symbol t is an integer from 0 to 8. In some embodiments, the symbol X^{a} is independently —Cl, —Br, —I, or —F. In some embodiments, p is 1. In some embodiments, p is 2. In some embodiments, q is 1. In some embodiments, q is 2. In some embodiments, r is 0. In some embodiments, r is 1. In some embodiments, r is 2. In some embodiments, r is 3. In some embodiments, r is 4. In some embodiments, m is 1. In some embodiments, m is 2. In some embodiments, v is 1. In some embodiments, v is 2. In some embodiments, n is 0. In some embodiments, n is 1. In some embodiments, n is 2. In some embodiments, n is 3. In some embodiments, n is 4. In some embodiments, t is 0. In some embodiments, t is 1. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, X^{a} is —Cl. In some embodiments, X^{a} is —Br. In some embodiments, X^{a} is —I. In some embodiments, X^{a} is —F. In some embodiments, X is —Cl. In some embodiments, X is —Br. In some embodiments, X is —I. In some embodiments, X is —F.

R¹⁴, R¹⁵, R¹⁶, and R¹⁷ are independently hydrogen, halogen, —CF₃₅—CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹⁴, R¹⁵, R¹⁶, and R¹⁷ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol t is independently an integer from 0 to 5. In some embodiments, t is 0. In some embodiments, t is 1. In some embodiments, t is 2. In some embodiments, t is 3. In some embodiments, t is 4. In some embodiments, t is 5. The symbol p is independently 1 or 2. In some embodiments, p is 1. In some embodiments, p is 2. The symbol q is independently an integer from 1 to 2. In some embodiments, q is 1. In some embodiments, q is 2. The symbol r is independently an integer from 0 to 4. In some embodiments, r is 0. In some embodiments, r is 1. In some embodiments, r is 2. In some embodiments, r is 3. In some embodiments, r is 4. Y is independently, —N═, or —N⁺(O⁻)═, or —C(R¹³)═. In some embodiments, Y is N═. In other embodiments, Y is —N⁺(O⁻)=. In some embodiments, Y is CH═. In some embodiments, Y is —C(R¹³)═. Where each R¹⁴, R¹⁵, R¹⁶, and R¹⁷, X^{a}, r, p, and q is different, they may be referred to, for example, as R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R¹³ is defined by R^{14c}, R^{15c}, R^{16c}, R^{17c}, X^{ac}, r^{c}, p^{c}, and q^{c}. In some embodiments, R¹³ is defined by R^{14d}, R^{15d}, R^{16d}, R^{17d}, X^{ad}, r^{d}, p^{d}, and q^{d}. In some embodiments, R¹³ is defined by R^{14e}, R^{15e}, R^{16e}, and R^{17e}, X^{ae}, r^{e}, p^{e}, and q^{e}. In some embodiments, R¹³ is defined by R^{14f}, R^{15f}, R^{16f}, and R^{17f}, X^{af}, r^{f}, p^{f}, and q^{f}. In some embodiments, R¹³ is defined by R^{14g}, R^{15g}, R^{16g}, and R^{17g}, X^{ag}, r^{g}, p^{g}, and q^{g}. Where c, d, e, f, and g denote substituents of different R¹³ respectively.

In some embodiments, the compound having formula I is a compound having the formula:

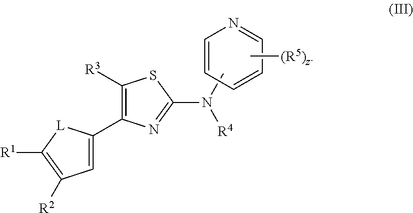

L, X, X^{a}, Y, m, n, p, q, r, t, v, R¹, R², R³, R⁴, R⁶, R⁷, R⁸, R⁹, R¹⁰, R¹¹, R¹², R¹³, R¹⁴, R¹⁵, R¹⁶, and R¹⁷ are as described herein (e.g. formula (I) and (II), including embodiments).

R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R⁵ substituents are joined to form a substituted or unsubstituted aryl. In some embodiments, two adjacent R⁵ substituents are joined to form a substituted or unsubstituted phenyl. In some embodiments, two adjacent R⁵ substituents are joined to form an unsubstituted phenyl.

R^{7b}, R^{8b}, R^{9b}, and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R^{7b}, R^{8b}, R^{9b}, and R^{10b} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Where each R^{7b}, R^{8b}, R^{9b}, and R^{10b}, X^{b}, n1, v1, and m1 is different, they may be referred to, for example, as R^{7bc}, R^{7bc}, R^{7be}, R^{7bf}, R^{7bg}, R^{8bc}, R^{8bd}, R^{8be}, R^{8bf}, R^{8bg}, R^{9bc}, R^{9bd}, R^{9be}, R^{9bf}, R_{9bg}, R^{10bc}, R^{10bc}, R^{10be}, R^{10bf}, R^{10bg}, X^{bc}, X^{bd}, X^{be}, X^{bf}, X^{bg}, n1^{c}, n1^{d}, n1^{e}, n1^{f}, n1^{g}, v1^{c}, v1^{d}, v1^{e}, v1^{f}, v1^{g}, m1^{c}, m1^{c}, m1^{d}, m1^{e}, m1^{f}, m1^{g}, and so on, wherein each R^{7bc}, R^{7bd}, R^{7be}, R^{7bf}, R^{7bg} is defined the same as R^{7b}, each R^{8bc}, R^{8bd}, R^{8be}, R^{8bf}, R^{8bg} is defined the same as R^{8b}, each R^{9bc}, R^{9bd}, R^{9be}, R^{9bf}, R^{9bg} is defined the same as R^{9b}, each R^{10bc}, R^{10bd}, R^{10be}, R^{10bf}, R^{10bg} is defined the same as R^{10b}, each X^{bc}, X^{bd}, X^{be}, X^{bf}, X^{bg} is defined the same as X^{b}, each n1^{c}, n1^{d}, n1^{e}, n1^{f}, n1^{g} is defined the same as n1, each v1^{c}, v1^{d}, v1^{e}, v1^{f}, v1^{g} is defined the same as v1, each m1^{c}, m1^{d}, m1^{f}, m1^{g} is defined the same as m1. In some embodiments, R⁵ is defined by R^{7bc}, R^{8bc}, R^{9bc}, R^{10bc}, X^{bc}, n1^{c}, v1^{c}, and m1^{c}. In some embodiments, R⁵ is defined by R^{7bd}, R^{8bd}, R^{9bd}, R^{10bd}, X^{bd}, n1^{d}, v1^{d}, m1^{d}. In some embodiments, R⁵ is defined by R^{7be}, R^{8be}, R^{9be}, R^{10be}, X^{be}, n1^{e}, v1^{e}, and m1^{e}. In some embodiments, R⁵ is defined by R^{7bf}, R^{8bf}, R^{9bf}, R^{10bf}, X^{bf}, n1^{f}, v1^{f} and m1^{f}. In some embodiments, R⁵ is defined by R^{7bg}, R^{8bg}, R^{9bg}, R^{10bg}, X^{bg}, n1^{g}, v1^{g}, and m1^{g}. Where c, d, e, f, and g denote substituents of different R⁵ respectively. The symbol z is independently an integer from 0 to 4. In some embodiments, z is 0. In some embodiments, z is 1. In some embodiments, z is 2. In some embodiments, z is 3. In some embodiments, z is 4. The symbol v1 is independently 1 or 2. In some embodiments, v1 is 1. In some embodiments, v1 is 2. The symbol m1 is independently an integer from 1 to 2. In some embodiments, m1 is 1. In some embodiments, m1 is 2. The symbol n1 is independently an integer from 0 to 4. In some embodiments, n1 is 0. In some embodiments, n1 is 1. In some embodiments, n1 is 2. In some embodiments, n1 is 3. In some embodiments, n1 is 4. X^{b} is independently —Cl, —Br, —I, or —F. In some embodiments, X^{b} is —Cl. In some embodiments, X^{b} is —Br. In some embodiments, X^{b} is —I. In some embodiments, X^{b} is —F.

In some embodiments, R⁵ is substituted or unsubstituted alkyl or substituted or unsubstituted heteroalkyl. In some embodiments, R⁵ is unsubstituted alkyl or substituted or unsubstituted heteroalkyl. In some embodiments, R⁵ is unsubstituted alkyl. In some embodiments, R⁵ is unsubstituted (C₁-C₄)alkyl. In some embodiments, R⁵ is methyl. In some embodiments, R⁵ is —OR^{10b} and R^{10b} is substituted or unsubstituted alkyl. In some embodiments, R^{10b} is substituted or unsubstituted (C₁-C₄)alkyl. In some embodiments, R^{10b} is methyl.

In some embodiments, the compound having formula I, II, or III, is a compound having the formula:

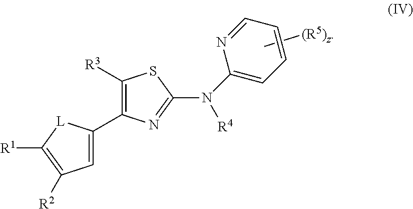

L, X, X^{a}, X^{b}, Y, m, m1, n, n1, p, q, r, t, v, v1, z, R¹, R², R³, R⁴, R⁵, R⁶, R⁷, R⁸, R⁹, R¹⁰, R^{7b}, R^{8b}, R^{9b}, R^{10b}, R¹¹, R¹², R¹³, R¹⁴, R¹⁵, R¹⁶, and R¹⁷ are as described herein (e.g. formula (I), (II), and (III), including embodiments).

In some embodiments, the compound having formula I, II, III, or IV, is a compound having formula:

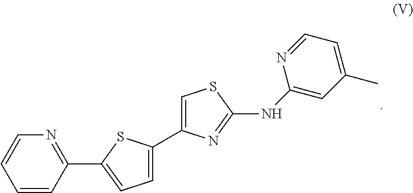 [SMILES: Cc1ccnc(Nc2nc(-c3ccc(-c4ccccn4)s3)cs2)c1]

In some embodiments, the compound having formula I, II, III, or IV, is a compound having formula:

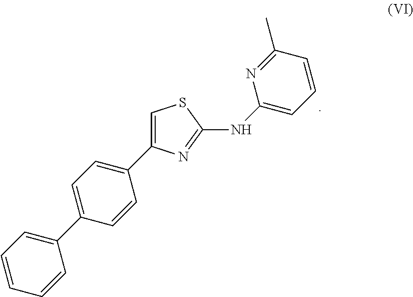 [SMILES: Cc1cccc(Nc2nc(-c3ccc(-c4ccccc4)cc3)cs2)n1]

In another aspect is a compound having the formula:

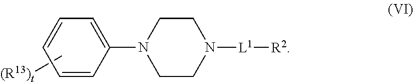

X, X^{a}, m, n, p, q, r, t, v, R², R⁷, R⁸, R⁹, R¹⁰, R¹³, R¹⁴, R¹⁵, R¹⁶, and R¹⁷ are as described herein (e.g. formula (I) to (VII), including embodiments). L¹ is a bond, substituted or unsubstituted alkylene, substituted or unsubstituted heteroalkylene, —NR^{1A}—, —O—, —S—, —C(O)—, or —CHR^{1A}—. R^{1A} is independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, L¹, R^{1A}, X, X^{a}, m, n, p, q, r, t, v, R², R⁷, R⁸, R⁹, R¹⁰, R¹³, R¹⁴, R¹⁵, R¹⁶, and R¹⁷, are as described in the paragraphs below in a compound of formula (VI). In some embodiments, these values are included in any other formula described herein. In some embodiments, R^{1A} and R² are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O), —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, the symbols m, p, q, and v are independently an integer from 1 to 2. In some embodiments, the symbols n and r are independently an integer from 0 to 4. In some embodiments, the symbol t is independently an integer from 0 to 8. In some embodiments, the symbols X and X^{a} are independently —Cl, —Br, —I, or —F.

In some embodiments, L¹ is a bond, substituted or unsubstituted alkylene, substituted or unsubstituted heteroalkylene, —NR^{1A}—, —O—, —S—, —C(O)—, or —CHR^{1A}—. In some embodiments, L¹ is a bond. In some embodiments, L¹ is a substituted alkylene. In some embodiments, L¹ is an unsubstituted alkylene. In some embodiments, L¹ is an unsubstituted (C₁-C₄) alkylene. In some embodiments, L¹ is a methylene. In some embodiments, L¹ is an ethylene. In some embodiments, L¹ is a propylene. In some embodiments, L¹ is a butylene. In some embodiments, L¹ is a substituted heteroalkylene. In some embodiments, L¹ is a heteroalkylene substituted with oxo, halogen, —CCl₃, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, L¹ is a heteroalkylene substituted with oxo and halogen. In some embodiments, L¹ is a heteroalkylene substituted with oxo. In some embodiments, L¹ is a heteroalkylene substituted with halogen. In some embodiments, L¹ is a heteroalkylene substituted with oxo and —Cl. In some embodiments, L¹ is —NHCH(CCl₃)CH₂C(O)—. In some embodiments, L¹ is an unsubstituted heteroalkylene. In some embodiments, L¹ is —NR^{1A}—. In some embodiments, L¹ is —NH—. In some embodiments, L¹ is —O—. In some embodiments, L¹ is —S—. In some embodiments, L¹ is —C(O)—. In some embodiments, L¹ is —CHR^{1A}—.

In some embodiments, R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R² is substituted or unsubstituted alkyl. In some embodiments, R² is substituted or unsubstituted heteroalkyl. In some embodiments, R² is substituted or unsubstituted cycloalkyl. In some embodiments, R² is substituted or unsubstituted heterocycloalkyl. In some embodiments, R² is substituted or unsubstituted aryl. In some embodiments, R² is substituted or unsubstituted heteroaryl. In some embodiments, R² is unsubstituted alkyl. In some embodiments, R² is unsubstituted heteroalkyl. In some embodiments, R² is unsubstituted cycloalkyl. In some embodiments, R² is unsubstituted heterocycloalkyl. In some embodiments, R² is unsubstituted aryl. In some embodiments, R² is unsubstituted heteroaryl. In some embodiments, R² is substituted alkyl. In some embodiments, R² is substituted heteroalkyl. In some embodiments, R² is substituted cycloalkyl. In some embodiments, R² is substituted heterocycloalkyl. In some embodiments, R² is substituted aryl. In some embodiments, R² is substituted heteroaryl. In some embodiments, R² is R²³-substituted alkyl. In some embodiments, R² is R²³-substituted heteroalkyl. In some embodiments, R² is R²³-substituted cycloalkyl. In some embodiments, R² is R²³-substituted heterocycloalkyl. In some embodiments, R² is R²³-substituted aryl. In some embodiments, R² is R²³-substituted heteroaryl. In some embodiments, R² is substituted or unsubstituted phenyl. In some embodiments, R² is R²³-substituted or unsubstituted phenyl. In some embodiments, R² is substituted phenyl. In some embodiments, R² is unsubstituted phenyl. In some embodiments, R² is R²³-substituted phenyl. In some embodiments, R² is substituted or unsubstituted thienyl. In some embodiments, R² is R²³-substituted or unsubstituted thienyl. In some embodiments, R² is substituted thienyl. In some embodiments, R² is unsubstituted thienyl. In some embodiments, R² is R²³-substituted thienyl. In some embodiments, R² is substituted or unsubstituted 4,5,6,7-tetrahydrobenzo[b]thienyl. In some embodiments, R² is R²³-substituted or unsubstituted 4,5,6,7-tetrahydrobenzo[b]thienyl. In some embodiments, R² is substituted 4,5,6,7-tetrahydrobenzo[b]thienyl. In some embodiments, R² is unsubstituted 4,5,6,7-tetrahydrobenzo[b]thienyl. In some embodiments, R² is R²³-substituted 4,5,6,7-tetrahydrobenzo[b]thienyl. In some embodiments, R² is substituted or unsubstituted pyridyl. In some embodiments, R² is R²³-substituted or unsubstituted pyridyl. In some embodiments, R² is substituted pyridyl. In some embodiments, R² is unsubstituted pyridyl. In some embodiments, R² is R²³-substituted pyridyl. In some embodiments, R² is substituted or unsubstituted 1,3,4-thiadiazolyl. In some embodiments, R² is R²³-substituted or unsubstituted 1,3,4-thiadiazolyl. In some embodiments, R² is substituted 1,3,4-thiadiazolyl. In some embodiments, R² is unsubstituted 1,3,4-thiadiazolyl. In some embodiments, R² is R²³-substituted 1,3,4-thiadiazolyl. In some embodiments, R² is substituted or unsubstituted piperidinyl. In some embodiments, R² is R²³-substituted or unsubstituted piperidinyl. In some embodiments, R² is substituted piperidinyl. In some embodiments, R² is unsubstituted piperidinyl. In some embodiments, R² is R²³-substituted piperidinyl. In some embodiments, R² is substituted or unsubstituted piperazinyl. In some embodiments, R² is R²³-substituted or unsubstituted piperazinyl. In some embodiments, R² is substituted piperazinyl. In some embodiments, R² is unsubstituted piperazinyl. In some embodiments, R² is R²³-substituted piperazinyl. In some embodiments, R² is substituted or unsubstituted oxazolyl. In some embodiments, R² is R²³-substituted or unsubstituted oxazolyl. In some embodiments, R² is substituted oxazolyl. In some embodiments, R² is unsubstituted oxazolyl. In some embodiments, R² is R²³-substituted oxazolyl. In some embodiments, R² is substituted or unsubstituted thiazolyl. In some embodiments, R² is R²³-substituted or unsubstituted thiazolyl. In some embodiments, R² is substituted thiazolyl. In some embodiments, R² is unsubstituted thiazolyl. In some embodiments, R² is R²³-substituted thiazolyl. In some embodiments, R² is R²³-substituted or unsubstituted benzo[d]oxazolyl. In some embodiments, R² is R²³-substituted benzo[d]oxazolyl. In some embodiments, R² is unsubstituted benzo[d]oxazolyl. In some embodiments, R² is substituted or unsubstituted tetrahydrothienyl. In some embodiments, R² is R²³-substituted or unsubstituted tetrahydrothienyl. In some embodiments, R² is substituted tetrahydrothienyl. In some embodiments, R² is unsubstituted tetrahydrothienyl. In some embodiments, R² is R²³-substituted tetrahydrothienyl. In some embodiments, R² is substituted or unsubstituted 2,3-dihydro-1H-pyrazolyl. In some embodiments, R² is R²³-substituted or unsubstituted 2,3-dihydro-1H-pyrazolyl. In some embodiments, R² is substituted 2,3-dihydro-1H-pyrazolyl. In some embodiments, R² is unsubstituted 2,3-dihydro-1H-pyrazolyl. In some embodiments, R² is R²³-substituted 2,3-dihydro-1H-pyrazolyl. In some embodiments, R² is substituted or unsubstituted (C₁-C₄) alkyl. In some embodiments, R² is R²³-substituted or unsubstituted (C₁-C₄) alkyl. In some embodiments, R² is substituted (C₁-C₄) alkyl. In some embodiments, R² is unsubstituted (C₁-C₄) alkyl. In some embodiments, R² is R²³-substituted (C₁-C₄) alkyl. In some embodiments, R² is substituted or unsubstituted ethyl. In some embodiments, R² is R²³-substituted or unsubstituted ethyl. In some embodiments, R² is substituted ethyl. In some embodiments, R² is unsubstituted ethyl. In some embodiments, R² is R²³-substituted ethyl.

In some embodiments, R² is R²³-substituted or unsubstituted imidazolyl. In some embodiments, R² is substituted imidazolyl. In some embodiments, R² is unsubstituted imidazolyl. In some embodiments, R² is R²³-substituted or unsubstituted 4H-1,2,4-triazolyl. In some embodiments, R² is substituted 4H-1,2,4-triazolyl. In some embodiments, R² is unsubstituted 4H-1,2,4-triazolyl. In some embodiments, R² is R²³-substituted or unsubstituted triazolyl. In some embodiments, R² is substituted triazolyl. In some embodiments, R² is unsubstituted triazolyl. In some embodiments, R² is substituted or unsubstituted naphthyl. In some embodiments, R² is substituted or unsubstituted furanyl. In some embodiments, R² is substituted or unsubstituted quinolinyl. In some embodiments, R² is unsubstituted naphthyl. In some embodiments, R² is unsubstituted furanyl. In some embodiments, R² is unsubstituted quinolinyl. In some embodiments, R² is substituted naphthyl. In some embodiments, R² is substituted furanyl. In some embodiments, R² is substituted quinolinyl. In some embodiments, R² is R²³-substituted naphthyl. In some embodiments, R² is R²³-substituted furanyl. In some embodiments, R² is R²³-substituted quinolinyl.

In some embodiments, R² is substituted with one R²³. In some embodiments, R² is substituted with two optionally different R²³. In some embodiments, R² is substituted with three optionally different R²³. In some embodiments, R² is substituted with four optionally different R²³. In some embodiments, two adjacent R²³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²³ substituents are joined to form a substituted cycloalkyl. In some embodiments, two adjacent R²³ substituents are joined to form an unsubstituted cycloalkyl. In some embodiments, two adjacent R²³ substituents are joined to form a substituted heterocycloalkyl. In some embodiments, two adjacent R²³ substituents are joined to form an unsubstituted heterocycloalkyl. In some embodiments, two adjacent R²³ substituents are joined to form a substituted aryl. In some embodiments, two adjacent R²³ substituents are joined to form an unsubstituted aryl. In some embodiments, two adjacent R²³ substituents are joined to form a substituted heteroaryl. In some embodiments, two adjacent R²³ substituents are joined to form an unsubstituted heteroaryl.

In some embodiments, R²³ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R²⁴-substituted or unsubstituted piperidinyl, R²⁴-substituted or unsubstituted piperazinyl, R²⁴-substituted or unsubstituted thiazolyl, R²⁴-substituted or unsubstituted oxazolyl, R²⁴-substituted or unsubstituted phenyl, R²⁴-substituted or unsubstituted thienyl, R²⁴-substituted or unsubstituted 4,5,6,7-tetrahydrobenzo[b]thienyl, R²⁴-substituted or unsubstituted pyridyl, or R²⁴-substituted or unsubstituted 1,3,4-thiadiazolyl. In some embodiments, R²³ is R²⁴-substituted or unsubstituted benzo[d]oxazolyl. In some embodiments, R²³ is R²⁴-substituted or unsubstituted imidazolyl. In some embodiments, R²³ is R²⁴-substituted imidazolyl. In some embodiments, R²³ is unsubstituted imidazolyl. In some embodiments, R²³ is R²⁴-substituted or unsubstituted 4H-1,2,4-triazolyl. In some embodiments, R²³ is R²⁴-substituted 4H-1,2,4-triazolyl. In some embodiments, R²³ is unsubstituted 4H-1,2,4-triazolyl. In some embodiments, R²³ is R²⁴-substituted or unsubstituted triazolyl. In some embodiments, R²³ is R²⁴-substituted triazolyl. In some embodiments, R²³ is unsubstituted triazolyl. In some embodiments, R²³ is independently R²⁴-substituted or unsubstituted alkyl, R²⁴-substituted or unsubstituted heteroalkyl, R²⁴-substituted or unsubstituted cycloalkyl, R²⁴ substituted or unsubstituted heterocycloalkyl, R²⁴-substituted or unsubstituted aryl, or R²⁴-substituted or unsubstituted heteroaryl. In some embodiments, R²³ is independently R²⁴-substituted alkyl. In some embodiments, R²³ is unsubstituted alkyl. In some embodiments, R²³ is R²⁴-substituted heteroalkyl. In some embodiments, R²³ is unsubstituted heteroalkyl. In some embodiments, R²³ is R²⁴-substituted cycloalkyl. In some embodiments, R²³ is unsubstituted cycloalkyl. In some embodiments, R²³ is R²⁴-substituted heterocycloalkyl. In some embodiments, R²³ is unsubstituted heterocycloalkyl. In some embodiments, R²³ is R²⁴-substituted aryl. In some embodiments, R²³ is unsubstituted aryl. In some embodiments, R²³ is R²⁴-substituted heteroaryl. In some embodiments, R²³ is unsubstituted heteroaryl. In some embodiments, R²³ is substituted with one R²⁴. In some embodiments, R²³ is substituted with two optionally different R²⁴. In some embodiments, R²³ is substituted with three optionally different R²⁴. In some embodiments, R²³ is substituted with four optionally different R²⁴. In some embodiments, R²³ is independently oxo. In some embodiments, R²³ is independently —Br. In some embodiments, R²³ is independently —F. In some embodiments, R²³ is independently —Cl. In some embodiments, R²³ is independently —I. In some embodiments, R²³ is independently —CH₃. In some embodiments, R²³ is independently —OCH₃. In some embodiments, R²³ is independently (C₁-C₄) alkyl. In some embodiments, R²³ is independently (C₁-C₈) alkyl. In some embodiments, R²³ is independently (C₇-C₁₀) alkyl. In some embodiments, R²³ is independently (C₆-C₁₂) alkyl. In some embodiments, R²³ is independently phenyl. In some embodiments, R²³ is independently —OH. In some embodiments, R²³ is independently —CF₃.

In some embodiments, two adjacent R²⁴ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁴ substituents are joined to form a substituted cycloalkyl. In some embodiments, two adjacent R²⁴ substituents are joined to form an unsubstituted cycloalkyl. In some embodiments, two adjacent R²⁴ substituents are joined to form a substituted heterocycloalkyl. In some embodiments, two adjacent R²⁴ substituents are joined to form an unsubstituted heterocycloalkyl. In some embodiments, two adjacent R²⁴ substituents are joined to form a substituted aryl. In some embodiments, two adjacent R²⁴ substituents are joined to form an unsubstituted aryl. In some embodiments, two adjacent R²⁴ substituents are joined to form a substituted heteroaryl. In some embodiments, two adjacent R²⁴ substituents are joined to form an unsubstituted heteroaryl.

In some embodiments, R²⁴ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R²⁴ is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, or R²⁵-substituted or unsubstituted heteroaryl. In some embodiments, R²⁴ is CH₃. In some embodiments, R²⁴ is independently R²⁵-substituted or unsubstituted piperidinyl, R²⁵-substituted or unsubstituted piperazinyl, R²⁵-substituted or unsubstituted thiazolyl, R²⁵-substituted or unsubstituted oxazolyl, R²⁵-substituted or unsubstituted phenyl, R²⁵-substituted or unsubstituted thienyl, R²⁵-substituted or unsubstituted 4,5,6,7-tetrahydrobenzo[b]thienyl, R²⁵-substituted or unsubstituted pyridyl, or R²⁵-substituted or unsubstituted 1,3,4-thiadiazolyl. In some embodiments, R²⁴ is R²⁵-substituted or unsubstituted benzo[d]oxazolyl. In some embodiments, R²⁴ is R²⁵-substituted or unsubstituted imidazolyl. In some embodiments, R²⁴ is R²⁵-substituted imidazolyl. In some embodiments, R²⁴ is unsubstituted imidazolyl. In some embodiments, R²⁴ is R²⁵-substituted or unsubstituted 4H-1,2,4-triazolyl. In some embodiments, R²⁴ is R²⁵-substituted 4H-1,2,4-triazolyl. In some embodiments, R²⁴ is unsubstituted 4H-1,2,4-triazolyl. In some embodiments, R²⁴ is R²⁵-substituted or unsubstituted triazolyl. In some embodiments, R²⁴ is R²⁵-substituted triazolyl. In some embodiments, R²⁴ is unsubstituted triazolyl. In some embodiments, R²⁴ is independently R²⁵-substituted or unsubstituted alkyl, R²⁵-substituted or unsubstituted heteroalkyl, R²⁵-substituted or unsubstituted cycloalkyl, R²⁵ substituted or unsubstituted heterocycloalkyl, R²⁵-substituted or unsubstituted aryl, or R²⁵-substituted or unsubstituted heteroaryl. In some embodiments, R²⁴ is independently R²⁵-substituted alkyl. In some embodiments, R²⁴ is unsubstituted alkyl. In some embodiments, R²⁴ is R²⁵-substituted heteroalkyl. In some embodiments, R²⁴ is unsubstituted heteroalkyl. In some embodiments, R²⁴ is R²⁵-substituted substituted cycloalkyl. In some embodiments, R²⁴ is unsubstituted cycloalkyl. In some embodiments, R²⁴ is R²⁵-substituted heterocycloalkyl. In some embodiments, R²⁴ is unsubstituted heterocycloalkyl. In some embodiments, R²⁴ is R²⁵-substituted aryl. In some embodiments, R²⁴ is unsubstituted aryl. In some embodiments, R²⁴ is R²⁵-substituted heteroaryl. In some embodiments, R²⁴ is unsubstituted heteroaryl. In some embodiments, R²⁴ is substituted with one R²⁵. In some embodiments, R²⁴ is substituted with two optionally different R²⁵. In some embodiments, R²⁴ is substituted with three optionally different R²⁵. In some embodiments, R²⁴ is substituted with four optionally different R²⁵. In some embodiments, R²⁴ is independently oxo. In some embodiments, R²⁴ is independently —Br. In some embodiments, R²⁴ is independently —F. In some embodiments, R²⁴ is independently —Cl. In some embodiments, R²⁴ is independently —I. In some embodiments, R²⁴ is independently —CH₃. In some embodiments, R²⁴ is independently —OCH₃. In some embodiments, R²⁴ is independently (C₁-C₄) alkyl. In some embodiments, R²⁴ is independently (C₁-C₈) alkyl. In some embodiments, R²⁴ is independently (C₇-C₁₀) alkyl. In some embodiments, R²⁴ is independently (C₆-C₁₂) alkyl. In some embodiments, R²⁴ is independently phenyl. In some embodiments, R²⁴ is independently —OH. In some embodiments, R²⁴ is independently —CF₃. In some embodiments, R²⁴ is R²⁵-substituted or unsubstituted benzo[d]oxazolyl. In some embodiments, R²⁴ is R²⁵-substituted benzo[d]oxazolyl. In some embodiments, R²⁴ is unsubstituted benzo[d]oxazolyl. In some embodiments, R²⁴ is R²⁵-substituted or unsubstituted imidazolyl. In some embodiments, R²⁴ is R²⁵-substituted imidazolyl. In some embodiments, R²⁴ is unsubstituted imidazolyl. In some embodiments, R²⁴ is R²⁵-substituted or unsubstituted 4H-1,2,4-triazolyl. In some embodiments, R²⁴ is R²⁵-substituted 4H-1,2,4-triazolyl. In some embodiments, R²⁴ is unsubstituted 4H-1,2,4-triazolyl. In some embodiments, R²⁴ is R²⁵-substituted or unsubstituted triazolyl. In some embodiments, R²⁴ is R²⁵-substituted triazolyl. In some embodiments, R²⁴ is unsubstituted triazolyl. In some embodiments, R²⁴ is R²⁵-substituted or unsubstituted (C₁-C₄) alkyl. In some embodiments, R²⁴ is 2-propyl. In some embodiments, R²⁴ is methyl. In some embodiments, R²⁴ is ethyl. In some embodiments, R²⁴ is propyl. In some embodiments, R²⁴ is butyl. In some embodiments, R²⁴ is cyclopropyl. In some embodiments, R²⁴ is cyclobutyl.

In some embodiments, R¹³ is independently hydrogen, oxo, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl.

In some embodiments, a compound of formula (VI) is substituted with one R¹³. In some embodiments, a compound of formula (VI) is substituted with two optionally different R¹³. In some embodiments, a compound of formula (VI) is substituted with three optionally different R¹³. In some embodiments, a compound of formula (VI) is substituted with four optionally different R¹³. In some embodiments, a compound of formula (VI) is substituted with five optionally different R¹³. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents are joined to form a substituted cycloalkyl. In some embodiments, two adjacent R¹³ substituents are joined to form an unsubstituted cycloalkyl. In some embodiments, two adjacent R¹³ substituents are joined to form a substituted heterocycloalkyl. In some embodiments, two adjacent R¹³ substituents are joined to form an unsubstituted heterocycloalkyl. In some embodiments, two adjacent R¹³ substituents are joined to form a substituted aryl. In some embodiments, two adjacent R¹³ substituents are joined to form an unsubstituted aryl. In some embodiments, two adjacent R¹³ substituents are joined to form a substituted heteroaryl. In some embodiments, two adjacent R¹³ substituents are joined to form an unsubstituted heteroaryl.

In some embodiments, R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R¹³ is independently halogen, —CX^{a}₃, —CN, —N(O)_{q}, —NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹³ is independently halogen, —CF₃, —CN, —N(O)₂, —NH₂, or —OH. In some embodiments, R¹³ is independently unsubstituted alkyl, unsubstituted heteroalkyl, unsubstituted cycloalkyl, unsubstituted heterocycloalkyl, unsubstituted aryl, or unsubstituted heteroaryl. In some embodiments, R¹³ is —Cl. In some embodiments, R¹³ is —Br. In some embodiments, R¹³ is —I. In some embodiments, R¹³ is —F. In some embodiments, R¹³ is independently two different halogens. In some embodiments, R¹³ is —CF₃. In some embodiments, R¹³ is —CN. In some embodiments, R¹³ is —N(O)₂. In some embodiments, R¹³ is —NH₂. In some embodiments, R¹³ is —OH. In some embodiments, R¹³ is C(O)CH₃. In some embodiments, R¹³ is —OCH₃. In some embodiments, R¹³ is unsubstituted phenyl.

In some embodiments, R¹³ is R⁵⁶-substituted or unsubstituted heteroaryl. In some embodiments, R¹³ is R⁵⁶-substituted heteroaryl. In some embodiments, R¹³ is unsubstituted heteroaryl. In some embodiments, R¹³ is R⁵⁶-substituted or unsubstituted piperidinyl. In some embodiments, R¹³ is R⁵⁶-substituted or unsubstituted piperazinyl. In some embodiments, R¹³ is R⁵⁶-substituted or unsubstituted thiazolyl. In some embodiments, R¹³ is R⁵⁶-substituted or unsubstituted oxazolyl. In some embodiments, R¹³ is R⁵⁶-substituted or unsubstituted phenyl. In some embodiments, R¹³ is R⁵⁶-substituted or unsubstituted thienyl. In some embodiments, R¹³ is R⁵⁶-substituted or unsubstituted 4,5,6,7-tetrahydrobenzo[b]thienyl. In some embodiments, R¹³ is R⁵⁶-substituted or unsubstituted pyridyl. In some embodiments, R¹³ is R⁵⁶-substituted or unsubstituted 1,3,4-thiadiazolyl. In some embodiments, R¹³ is R⁵⁶-substituted or unsubstituted benzo[d]oxazolyl. In some embodiments, R¹³ is R⁵⁶-substituted or unsubstituted imidazolyl. In some embodiments, R¹³ is R⁵⁶-substituted imidazolyl. In some embodiments, R¹³ is unsubstituted imidazolyl. In some embodiments, R¹³ is R⁵⁶-substituted or unsubstituted 4H-1,2,4-triazolyl. In some embodiments, R¹³ is R⁵⁶-substituted 4H-1,2,4-triazolyl. In some embodiments, R¹³ is unsubstituted 4H-1,2,4-triazolyl. In some embodiments, R¹³ is R⁵⁶-substituted or unsubstituted triazolyl. In some embodiments, R¹³ is R⁵⁶-substituted triazolyl. In some embodiments, R¹³ is unsubstituted triazolyl.

In some embodiments, R⁵⁶ is independently halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R⁵⁶ is independently R⁵⁷-substituted or unsubstituted alkyl, R⁵⁷-substituted or unsubstituted heteroalkyl, R⁵⁷-substituted or unsubstituted cycloalkyl, R⁵⁷ substituted or unsubstituted heterocycloalkyl, R⁵⁷-substituted or unsubstituted aryl, or R⁵⁷-substituted or unsubstituted heteroaryl. In some embodiments, R⁵⁶ is —CH₃.

In some embodiments, R¹³ is substituted with one R⁵⁶. In some embodiments, R¹³ is substituted with two optionally different R⁵⁶. In some embodiments, R¹³ is substituted with three optionally different R⁵⁶. In some embodiments, R¹³ is substituted with four optionally different R⁵⁶. In some embodiments, R¹³ is substituted with five optionally different R⁵⁶. In some embodiments, two adjacent R⁵⁶ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R⁵⁶ substituents are joined to form a substituted cycloalkyl. In some embodiments, two adjacent R⁵⁶ substituents are joined to form an unsubstituted cycloalkyl. In some embodiments, two adjacent R⁵⁶ substituents are joined to form a substituted heterocycloalkyl. In some embodiments, two adjacent R⁵⁶ substituents are joined to form an unsubstituted heterocycloalkyl. In some embodiments, two adjacent R⁵⁶ substituents are joined to form a substituted aryl. In some embodiments, two adjacent R⁵⁶ substituents are joined to form an unsubstituted aryl. In some embodiments, two adjacent R⁵⁶ substituents are joined to form a substituted heteroaryl. In some embodiments, two adjacent R⁵⁶ substituents are joined to form an unsubstituted heteroaryl.

In some embodiments, R¹³ is —NR¹⁴R¹⁵. In some embodiments, R¹⁵ is R⁶²-substituted or unsubstituted heteroaryl. In some embodiments, R¹⁵ is R⁶²-substituted heteroaryl. In some embodiments, R¹⁵ is unsubstituted heteroaryl. In some embodiments, R¹⁵ is R⁶²-substituted or unsubstituted piperidinyl, R⁶²-substituted or unsubstituted piperazinyl, R⁶²-substituted or unsubstituted thiazolyl, R⁶²-substituted or unsubstituted oxazolyl, R⁶²-substituted or unsubstituted phenyl, R⁶²-substituted or unsubstituted thienyl, R⁶²-substituted or unsubstituted 4,5,6,7-tetrahydrobenzo[b]thienyl, R⁶²-substituted or unsubstituted pyridyl, or R⁶²-substituted or unsubstituted 1,3,4-thiadiazolyl. In some embodiments, R¹⁵ is R⁶²-substituted or unsubstituted benzo[d]oxazolyl. In some embodiments, R¹⁵ is R⁶²-substituted or unsubstituted imidazolyl. In some embodiments, R¹⁵ is R⁶²-substituted imidazolyl. In some embodiments, R¹⁵ is unsubstituted imidazolyl. In some embodiments, R¹⁵ is R⁶²-substituted or unsubstituted 4H-1,2,4-triazolyl. In some embodiments, R¹⁵ is R⁶²-substituted 4H-1,2,4-triazolyl. In some embodiments, R¹⁵ is unsubstituted 4H-1,2,4-triazolyl. In some embodiments, R¹⁵ is R⁶²-substituted or unsubstituted triazolyl. In some embodiments, R¹⁵ is R⁶²-substituted triazolyl. In some embodiments, R¹⁵ is unsubstituted triazolyl. In some embodiments, R¹⁵ is R⁶²-substituted or unsubstituted benzo[g]quinolone. In some embodiments, R¹⁴ is R⁵⁹-substituted or unsubstituted benzo[g]quinolone. In some embodiments, R¹⁴ is R⁵⁹-substituted or unsubstituted heteroaryl. In some embodiments, R¹⁴ is R⁵⁹-substituted heteroaryl. In some embodiments, R¹⁴ is unsubstituted heteroaryl. In some embodiments, R¹⁴ is R⁵⁹-substituted or unsubstituted piperidinyl, R⁵⁹-substituted or unsubstituted piperazinyl, R⁵⁹-substituted or unsubstituted thiazolyl, R⁵⁹-substituted or unsubstituted oxazolyl, R⁵⁹-substituted or unsubstituted phenyl, R⁵⁹-substituted or unsubstituted thienyl, R⁵⁹-substituted or unsubstituted 4,5,6,7-tetrahydrobenzo[b]thienyl, R⁵⁹-substituted or unsubstituted pyridyl, or R⁵⁹-substituted or unsubstituted 1,3,4-thiadiazolyl. In some embodiments, R¹⁴ is R⁵⁹-substituted R⁵⁹-substituted or unsubstituted benzo[d]oxazolyl. In some embodiments, R¹⁴ is R⁵⁹-substituted or unsubstituted imidazolyl. In some embodiments, R¹⁴ is R⁵⁹-substituted imidazolyl. In some embodiments, R¹⁴ is unsubstituted imidazolyl. In some embodiments, R¹⁴ is R⁵⁹-substituted or unsubstituted 4H-1,2,4-triazolyl. In some embodiments, R¹⁴ is R⁵⁹-substituted 4H-1,2,4-triazolyl. In some embodiments, R¹⁴ is unsubstituted 4H-1,2,4-triazolyl. In some embodiments, R¹⁴ is R⁵⁹-substituted or unsubstituted triazolyl. In some embodiments, R¹⁴ is R⁵⁹-substituted triazolyl. In some embodiments, R¹⁴ is unsubstituted triazolyl.

In some embodiments, the symbols p and q are independently an integer from 1 to 2. In some embodiments, the symbol r is independently an integer from 0 to 4. In some embodiments, the symbol t is an integer from 0 to 8. In some embodiments, the symbol X^{a} is independently —Cl, —Br, —I, or —F. In some embodiments, p is 1. In some embodiments, p is 2. In some embodiments, q is 1. In some embodiments, q is 2. In some embodiments, r is 0. In some embodiments, r is 1. In some embodiments, r is 2. In some embodiments, r is 3. In some embodiments, r is 4. In some embodiments, m is 1. In some embodiments, m is 2. In some embodiments, v is 1. In some embodiments, v is 2. In some embodiments, n is 0. In some embodiments, n is 1. In some embodiments, n is 2. In some embodiments, n is 3. In some embodiments, n is 4. In some embodiments, t is 0. In some embodiments, t is 1. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, X^{a} is —Cl. In some embodiments, X^{a} is —Br. In some embodiments, X^{a} is —I. In some embodiments, X^{a} is —F. In some embodiments, X is —Cl. In some embodiments, X is —Br. In some embodiments, X is —I. In some embodiments, X is —F. Where each R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R¹³ is defined by R^{14c}, R^{15c}, R^{16c}, R^{17c}, X^{ac}, r^{c}, p^{c}, and q^{c}. In some embodiments, R¹³ is defined by R^{14d}, R^{15d}, R^{16d}, R^{17d}, X^{ad}, r^{c}, p^{c}, and q^{c}. In some embodiments, R¹³ is defined by R^{14e}, R^{15e}, R^{16e}, and R^{17e}, X^{ae}, r^{e}, p^{e}, and q^{e}. In some embodiments, R¹³ is defined by R^{14f}, R^{15f}, R^{16f}, and R^{17f}, X^{af}, r^{f}, p^{f}, and q^{f}. In some embodiments, R¹³ is defined by R^{14g}, R^{15g}, R^{16g}, and R^{17g}, X^{ag}, r^{g}, p^{g}, and q^{g}. Where c, d, e, f, and g denote substituents of different R¹³ respectively.

In some embodiments, the compound having formula (VII) is a compound having the formula:

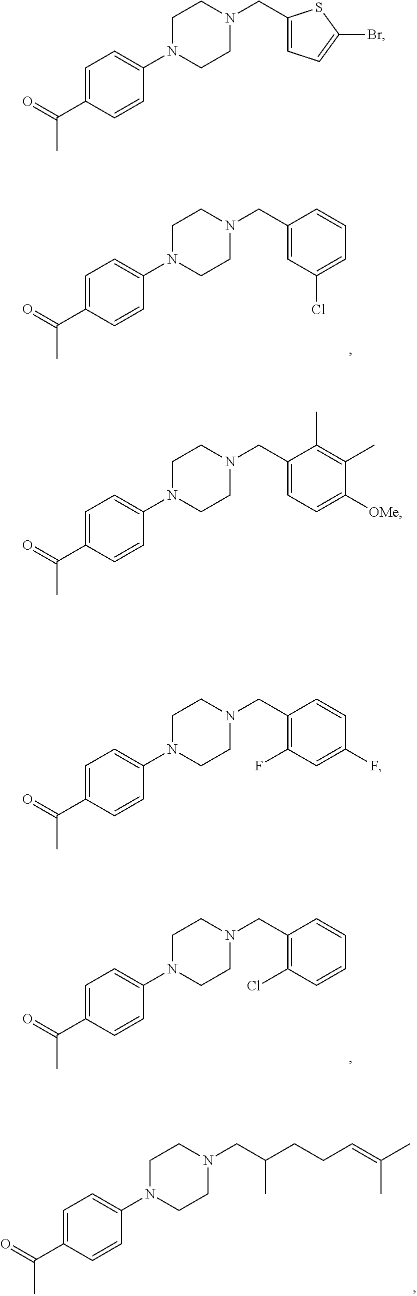 [SMILES: CC(=O)c1ccc(N2CCN(Cc3ccc(Br)s3)CC2)cc1 ; CC(=O)c1ccc(N2CCN(Cc3cccc(Cl)c3)CC2)cc1 ; COc1ccc(CN2CCN(c3ccc(C(C)=O)cc3)CC2)c(C)c1C ; CC(=O)c1ccc(N2CCN(Cc3ccc(F)cc3F)CC2)cc1 ; CC(=O)c1ccc(N2CCN(Cc3ccccc3Cl)CC2)cc1 ; CC(=O)c1ccc(N2CCN(CC(C)CCC=C(C)C)CC2)cc1]

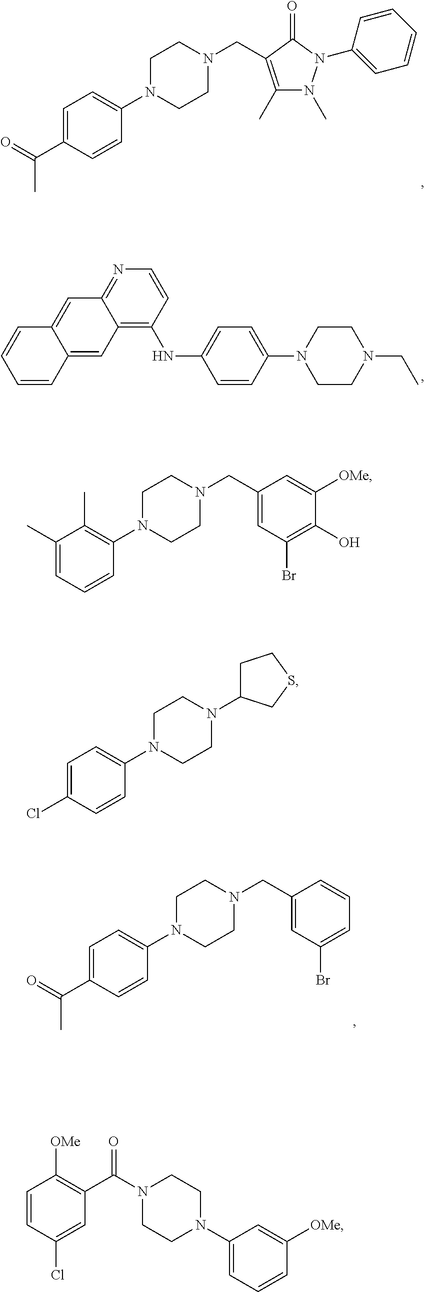

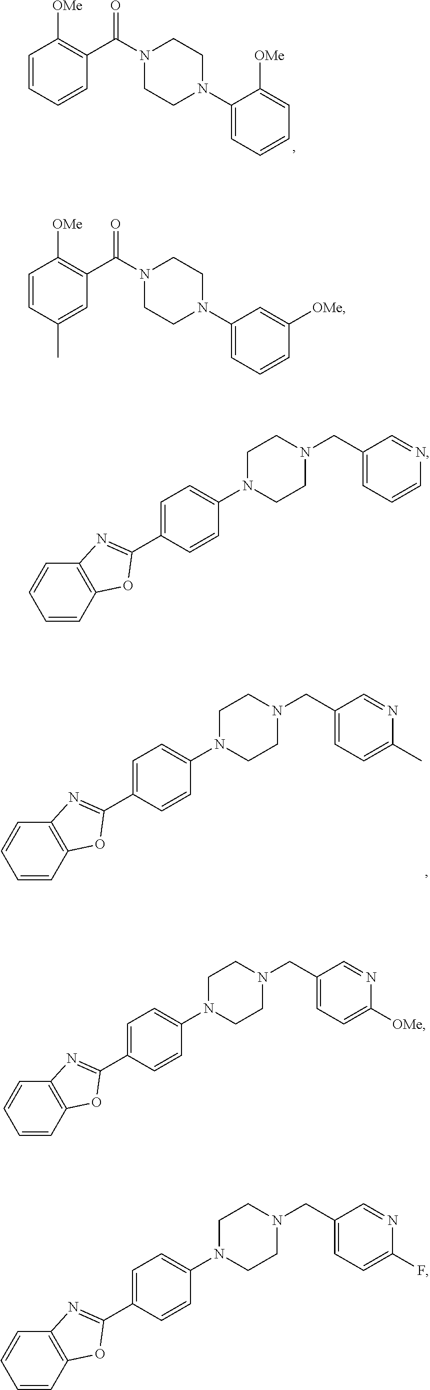 [SMILES: COc1ccccc1C(=O)N1CCN(c2ccccc2OC)CC1 ; COc1cccc(N2CCN(C(=O)c3cc(C)ccc3OC)CC2)c1 ; c1cncc(CN2CCN(c3ccc(-c4nc5ccccc5o4)cc3)CC2)c1 ; Cc1ccc(CN2CCN(c3ccc(-c4nc5ccccc5o4)cc3)CC2)cn1 ; COc1ccc(CN2CCN(c3ccc(-c4nc5ccccc5o4)cc3)CC2)cn1 ; Fc1ccc(CN2CCN(c3ccc(-c4nc5ccccc5o4)cc3)CC2)cn1]

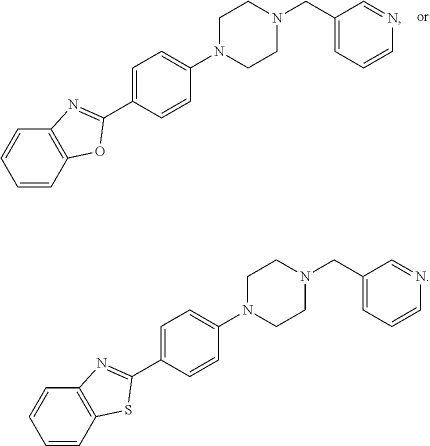 [SMILES: c1cncc(CN2CCN(c3ccc(-c4nc5ccccc5o4)cc3)CC2)c1 ; "or" ; c1cncc(CN2CCN(c3ccc(-c4nc5ccccc5s4)cc3)CC2)c1]

In some embodiments, the compound having formula (VII) is a compound having the formula:

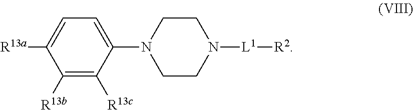

L¹, R^{1A}, X, X^{a}, m, n, p, q, r, t, v, R², R⁷, R⁸, R⁹, R¹⁰, R¹³, R¹⁴, R¹⁵, R¹⁶, and R¹⁷ are as described herein (e.g. formula (I) to (VII), including embodiments).

R^{13a}, R^{13b}, and R^{13e} are independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Where each R¹⁴, R¹⁵, R¹⁶, and R¹⁷ is different they may be referred to for example as R¹⁴′, R¹⁴″, and so on. R^{13a} and R^{13b} or R^{13b} and R^{13c} may optionally be joined to form a substituted or unsubstituted cycloalkyl, substituted or unsubstituted heterocycloalkyl, substituted or unsubstituted aryl, or substituted or unsubstituted heteroaryl. Where each R¹⁴, R¹⁵, R¹⁶, and R¹⁷, X^{a}, r, p, and q is different, they may be referred to, for example, as R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R^{13a} is defined by R^{14c}, R^{15c}, R^{16c}, R^{17c}, X^{ac}, r^{c}, p^{c}, and q^{c}. In some embodiments, R^{13b} is defined by R^{14d}, R^{15d}, R^{16d}, R^{17d}, X^{ad}, r^{d}, p^{d}, and q^{d}. In some embodiments, R^{13c} is defined by R^{14e}, R^{15e}, R^{16e}, and R^{17e}, X^{ae}, r^{e}, p^{e}, and q^{e}. Where c, d, and e denote substituents of R^{13a}, R^{13b}, and R^{13c} respectively.

In some embodiments, the compound having formula (VII) is a compound having the formula:

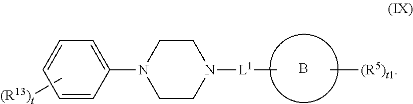

L¹, R^{1A}, X, X^{a}, m, n, p, q, r, t, v, R², R⁷, R⁸, R⁹, R¹⁰, R^{7b}, R^{8b}, R^{9b}, R^{10b}, R¹³, R¹⁴, R¹⁵, R¹⁶, and R¹⁷ are as described herein (e.g. formula (I) to (VIII), including embodiments).
Ring B is substituted or unsubstituted cycloalkyl, substituted or unsubstituted heterocycloalkyl, substituted or unsubstituted aryl, or substituted or unsubstituted heteroaryl.

The symbol t1 is independently an integer from 0 to 8. Where each R^{7b}, R^{8b}, R^{9b}, and R^{10b}, X^{b}, n1, v1, and m1 is different, they may be referred to, for example, as R^{7bc}, R^{7bd}, R^{7be}, R^{7bf}, R^{7bg}, R^{8bc}, R^{8bd}, R^{8be}, R^{8bf}, R^{8bg}, R^{9bc}, R^{9bd}, R^{9be}, R^{9bf}, R_{9bg}, R^{10bc}, R^{10bc}, R^{10be}, R^{10bf}, R^{10bg}, X^{bc}, X^{bd}, X^{be}, X^{bf}, X^{bg}, n1^{c}, n1^{d}, n1^{e}, n1^{f}, n1^{g}, v1^{c}, v1^{d}, v1^{e}, v1^{f}, v1^{g}, m1^{c}, m1^{d}, m1^{e}, m1^{f}, m1^{g}, and so on, wherein each R^{7bc}, R^{7bd}, R^{7be}, R^{7bf}, R^{7bg} is defined the same as R^{7b}, each R^{8bc}, R^{8bd}, R^{8be}, R^{8bf}, R^{8bg} is defined the same as R^{8b}, each R^{9bc}, R^{9bd}, R^{9be}, R^{9bf}, R^{9bg} is defined the same as R^{9b}, each R^{10bc}, R^{10bd}, R^{10be}, R^{10bf}, R^{10bg} is defined the same as R^{10b}, each X^{bc}, X^{bd}, X^{be}, X^{bf}, X^{bg} is defined the same as X^{b}, each n1^{c}, n1^{d}, n1^{e}, n1^{f}, n1^{g} is defined the same as n1, each v1^{c}, v1^{d}, v1^{e}, v1^{f}, v1^{g} is defined the same as v1, each m1^{c}, m1^{d}, m1^{e}, m1^{f}, m1^{g} is defined the same as m1. In some embodiments, R⁵ is defined by R^{7bc}, R^{8bc}, R^{9bc}, R^{10bc}, X^{bc}, n1^{c}, v1^{c}, and m1^{c}. In some embodiments, R⁵ is defined by R^{7bd}, R^{8bd}, R^{9bd}, R^{10bd}, X^{bd}, n1^{d}, v1^{d}, m1^{d}. In some embodiments, R⁵ is defined by R^{7be}, R^{8be}, R^{9be}, R^{10be}, X^{be}, n1^{e}, v1^{e}, and m1^{e}. In some embodiments, R⁵ is defined by R^{7bf}, R^{8bf}, R^{9bf}, R^{10bf}, X^{bf}, n1^{f}, v1^{f} and m1^{f}. In some embodiments, R⁵ is defined by R^{7bg}, R^{8bg}, R^{9bg}, R^{10bg}, X^{bg}, n1^{g}, v1^{g}, and m1^{g}. Where c, d, e, f, and g denote substituents of different R⁵ respectively.

In some embodiments, the compound having formula (IX) is a compound having the formula:

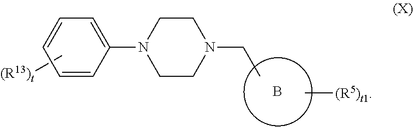

Ring B, L¹, R^{1A}, X, X^{a}, X^{b}, m, n, p, q, r, t, v, m1, v1, n1, t1, R², R⁵, R⁷, R⁸, R⁹, R¹⁰, R^{7b}, R^{8b}, R^{9b}, R^{10b}, R¹³, R¹⁴, R¹⁵, R¹⁶, and R¹⁷ are as described herein (e.g. formula (I) to (IX), including embodiments).

In another aspect is a compound having the formula:

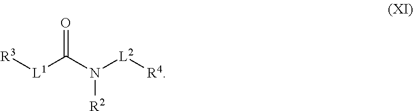

L¹, R^{1A}, X, m, n, v, R², R³, R⁴, R⁷, R⁸, R⁹, and R¹⁰ are as described herein (e.g. formula (I) to (X), including embodiments). L² is a bond, substituted or unsubstituted alkylene, substituted or unsubstituted heteroalkylene, —NR^{1B}, —O—, —S—, —C(O)—, or —CHR^{1B}—.

R^{1B} is independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, L¹, L², R^{1A}, R^{1B}, X, m, n, v, R², R³, R⁴, R⁷, R⁸, R⁹, and R¹⁰, are as described in the paragraphs below in a compound of formula (XI). In some embodiments, these values are included in any other formula described herein. In some embodiments, L¹ is a bond, substituted or unsubstituted alkylene, substituted or unsubstituted heteroalkylene, —NR^{1A}, —O—, —S—, —C(O)—, or —CHR^{1A}—. In some embodiments, L¹ is a bond. In some embodiments, L¹ is a substituted alkylene. In some embodiments, L¹ is an unsubstituted alkylene. In some embodiments, L¹ is an unsubstituted (C₁-C₄) alkylene. In some embodiments, L¹ is a methylene. In some embodiments, L¹ is an ethylene. In some embodiments, L¹ is a propylene. In some embodiments, L¹ is a butylene. In some embodiments, L¹ is a substituted heteroalkylene. In some embodiments, L¹ is a heteroalkylene substituted with oxo, halogen, —CCl₃, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, L¹ is a heteroalkylene substituted with oxo and halogen. In some embodiments, L¹ is a heteroalkylene substituted with oxo. In some embodiments, L¹ is a heteroalkylene substituted with halogen. In some embodiments, L¹ is a heteroalkylene substituted with oxo and —Cl. In some embodiments, L¹ is —NHCH(CCl₃)CH₂C(O)—. In some embodiments, L¹ is an unsubstituted heteroalkylene. In some embodiments, L¹ is —NR^{1A}—. In some embodiments, L¹ is —NH—. In some embodiments, L¹ is —O—. In some embodiments, L¹ is —S—. In some embodiments, L¹ is —C(O)—. In some embodiments, L¹ is —CHR^{1A}—. In some embodiments, L² is a bond. In some embodiments, L² is a substituted alkylene. In some embodiments, L² is an unsubstituted alkylene. In some embodiments, L² is an unsubstituted (C₁-C₄) alkylene. In some embodiments, L² is a methylene. In some embodiments, L² is an ethylene. In some embodiments, L² is a propylene. In some embodiments, L² is a butylene. In some embodiments, L² is a substituted heteroalkylene. In some embodiments, L² is a heteroalkylene substituted with oxo, halogen, —CCl₃, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, L² is a heteroalkylene substituted with oxo and halogen. In some embodiments, L² is a heteroalkylene substituted with oxo. In some embodiments, L² is a heteroalkylene substituted with halogen. In some embodiments, L² is a heteroalkylene substituted with oxo and —Cl. In some embodiments, L² is —NHCH(CCl₃)CH₂C(O)—. In some embodiments, L² is an unsubstituted heteroalkylene. In some embodiments, L² is —NH—. In some embodiments, L² is —O—. In some embodiments, L² is —S—. In some embodiments, L² is —C(O)—. In some embodiments, L² is —NR^{1B}—. In some embodiments, L² is —O—. In some embodiments, L² is —S—. In some embodiments, L² is —C(O)—. In some embodiments, L² is —CHR^{1B}—. In some embodiments, R^{1A}, R^{1B}, R², R³, and R⁴ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, the symbols m and v are independently an integer from 1 to 2. In some embodiments, the symbol n is independently an integer from 0 to 4. In some embodiments, the symbol X is independently —Cl, —Br, —I, or —F. In some embodiments, the symbol m is 1. In some embodiments, the symbol m is 2. In some embodiments, the symbol v is 1. In some embodiments, the symbol v is 2. In some embodiments, the symbol n is 0. In some embodiments, the symbol n is 1. In some embodiments, the symbol n is 2. In some embodiments, the symbol n is 3. In some embodiments, the symbol n is 4. In some embodiments, X is Cl. In some embodiments, X is Br. In some embodiments, X is —I. In some embodiments, X is —F.

In some embodiments, R³ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R³ is substituted or unsubstituted alkyl. In some embodiments, R³ is substituted or unsubstituted heteroalkyl. In some embodiments, R³ is substituted or unsubstituted cycloalkyl. In some embodiments, R³ is substituted or unsubstituted heterocycloalkyl. In some embodiments, R³ is substituted or unsubstituted aryl. In some embodiments, R³ is substituted or unsubstituted heteroaryl. In some embodiments, R³ is unsubstituted alkyl. In some embodiments, R³ is unsubstituted heteroalkyl. In some embodiments, R³ is unsubstituted cycloalkyl. In some embodiments, R³ is unsubstituted heterocycloalkyl. In some embodiments, R³ is unsubstituted aryl. In some embodiments, R³ is unsubstituted heteroaryl. In some embodiments, R³ is substituted alkyl. In some embodiments, R³ is substituted heteroalkyl. In some embodiments, R³ is substituted cycloalkyl. In some embodiments, R³ is substituted heterocycloalkyl. In some embodiments, R³ is substituted aryl. In some embodiments, R³ is substituted heteroaryl. In some embodiments, R³ is R²⁶-substituted alkyl. In some embodiments, R³ is R²⁶-substituted heteroalkyl. In some embodiments, R³ is R²⁶-substituted cycloalkyl. In some embodiments, R³ is R²⁶-substituted heterocycloalkyl. In some embodiments, R³ is R²⁶-substituted aryl. In some embodiments, R³ is R²⁶-substituted heteroaryl. In some embodiments, R³ is substituted or unsubstituted phenyl. In some embodiments, R³ is substituted or unsubstituted naphthyl. In some embodiments, R³ is substituted or unsubstituted thienyl. In some embodiments, R³ is substituted or unsubstituted furanyl. In some embodiments, R³ is substituted or unsubstituted pyridyl (pyridinyl). In some embodiments, R³ is substituted or unsubstituted quinolinyl. In some embodiments, R³ is unsubstituted phenyl. In some embodiments, R³ is unsubstituted naphthyl. In some embodiments, R³ is unsubstituted thienyl. In some embodiments, R³ is unsubstituted furanyl. In some embodiments, R³ is unsubstituted pyridyl (pyridinyl). In some embodiments, R³ is unsubstituted quinolinyl. In some embodiments, R³ is substituted phenyl. In some embodiments, R³ is substituted naphthyl. In some embodiments, R³ is substituted thienyl. In some embodiments, R³ is substituted furanyl. In some embodiments, R³ is substituted pyridyl (pyridinyl). In some embodiments, R³ is substituted quinolinyl. In some embodiments, R³ is R²⁶-substituted phenyl. In some embodiments, R³ is R²⁶-substituted naphthyl. In some embodiments, R³ is R²⁶-substituted thienyl. In some embodiments, R³ is R²⁶-substituted furanyl. In some embodiments, R³ is R²⁶-substituted pyridyl (pyridinyl). In some embodiments, R³ is R²⁶-substituted quinolinyl. In some embodiments, R²⁶ is oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, ONH₂, or —NHC═(O)NHNH₂.

In some embodiments, R³ is substituted with one R²⁶. In some embodiments, R³ is substituted with two optionally different R²⁶. In some embodiments, R³ is substituted with three optionally different R²⁶. In some embodiments, R³ is substituted with four optionally different R²⁶. In some embodiments, two adjacent R²⁶ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁶ substituents are joined to form a substituted cycloalkyl. In some embodiments, two adjacent R²⁶ substituents are joined to form an unsubstituted cycloalkyl. In some embodiments, two adjacent R²⁶ substituents are joined to form a substituted heterocycloalkyl. In some embodiments, two adjacent R²⁶ substituents are joined to form an unsubstituted heterocycloalkyl. In some embodiments, two adjacent R²⁶ substituents are joined to form a substituted aryl. In some embodiments, two adjacent R²⁶ substituents are joined to form an unsubstituted aryl. In some embodiments, two adjacent R²⁶ substituents are joined to form a substituted heteroaryl. In some embodiments, two adjacent R²⁶ substituents are joined to form an unsubstituted heteroaryl.

In some embodiments, R²⁶ is oxo, halogen, —CH₂OH, —CH₃, —OCH₃, —CF₃, —OCF₃, —CN, —OH, —NH₂, —COOH, —CONH₂, —NO₂, —SH, —SO₂Cl, —SO₃H, —SO₄H, —SO₂NH₂, —NHNH₂, —ONH₂, or NHC═(O)NHNH₂. In some embodiments, R²⁶ is —Cl, —Br, —I, —F, —CH₂OH, —CH₃, —OCH₃, —CF₃, —OCF₃, —OH, —NH₂, or —NO₂. In some embodiments, R²⁶ is —NO₂. In some embodiments, R²⁶ is —OCH₃. In some embodiments, R²⁶ is —OH. In some embodiments, R²⁶ is —F. In some embodiments, R²⁶ is —Cl. In some embodiments, R²⁶ is —CH₂OH. In some embodiments, R²⁶ is —CF₃. In some embodiments, R²⁶ is —OCF₃. In some embodiments, R²⁶ is —CH₃. In some embodiments, R²⁶ is unsubstituted alkyl, unsubstituted heteroalkyl, unsubstituted cycloalkyl, unsubstituted heterocycloalkyl, unsubstituted aryl, or unsubstituted heteroaryl. In some embodiments, R²⁶ is R²⁷-substituted alkyl, R²⁷-substituted heteroalkyl, R²⁷-substituted cycloalkyl, R²⁷ substituted heterocycloalkyl, R²⁷-substituted aryl, or R²⁷-substituted heteroaryl. In some embodiments, R²⁶ is unsubstituted alkyl. In some embodiments, R²⁶ is unsubstituted heteroalkyl. In some embodiments, R²⁶ is unsubstituted cycloalkyl. In some embodiments, R²⁶ is unsubstituted heterocycloalkyl. In some embodiments, R²⁶ is unsubstituted aryl. In some embodiments, R²⁶ is unsubstituted heteroaryl. In some embodiments, R²⁶ is R²⁷-substituted alkyl. In some embodiments, R²⁶ is R²⁷-substituted heteroalkyl. In some embodiments, R²⁶ is R²⁷-substituted cycloalkyl. In some embodiments, R²⁶ is R²⁷ substituted heterocycloalkyl. In some embodiments, R²⁶ is R²⁷-substituted aryl. In some embodiments, R²⁶ is R²⁷⁻substituted heteroaryl. In some embodiments, R²⁶ is R²⁷-substituted or unsubstituted phenyl. In some embodiments, R²⁶ is R²⁷-substituted or unsubstituted piperazinyl. In some embodiments, R²⁶ is R²⁷-substituted or unsubstituted piperidinyl. In some embodiments, R²⁶ is R²⁷-substituted or unsubstituted pyridinyl (pyridinyl). In some embodiments, R³ is substituted with one R²⁶. In some embodiments, R³ is substituted with two optionally different R²⁶. In some embodiments, R³ is substituted with three optionally different R²⁶. In some embodiments, R³ is substituted with four optionally different R²⁶. In some embodiments, R²⁷ is oxo, halogen, —CH₂OH, —CH₃, —OCH₃, —CF₃, —OCF₃, —CN, —OH, —NH₂, —COOH, —CONH₂, —NO₂, —SH, —SO₂Cl, —SO₃H, —SO₄H, —SO₂NH₂, —NHNH₂, —ONH₂, or —NHC═(O)NHNH₂. In some embodiments, R²⁷ is R²⁸-substituted or unsubstituted alkyl, R²⁸-substituted or unsubstituted heteroalkyl, R²⁸-substituted or unsubstituted cycloalkyl, R²⁸-substituted or unsubstituted heterocycloalkyl, R²⁸-substituted or unsubstituted aryl, or R²⁸-substituted or unsubstituted heteroaryl. In some embodiments, R²⁷ is unsubstituted phenyl. In some embodiments, R²⁷ is substituted phenyl. In some embodiments, R²⁷ is R²⁸-substituted or unsubstituted aryl. In some embodiments, R²⁷ is R²⁸-substituted or unsubstituted alkyl. In some embodiments, R²⁷ is —CH₃. In some embodiments, R²⁶ is substituted with one R²⁷. In some embodiments, R²⁶ is substituted with two optionally different R²⁷. In some embodiments, R²⁶ is substituted with three optionally different R²⁷. In some embodiments, R²⁶ is substituted with four optionally different R²⁷.

In some embodiments, two adjacent R²⁷ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁷ substituents are joined to form a substituted cycloalkyl. In some embodiments, two adjacent R²⁷ substituents are joined to form an unsubstituted cycloalkyl. In some embodiments, two adjacent R²⁷ substituents are joined to form a substituted heterocycloalkyl. In some embodiments, two adjacent R²⁷ substituents are joined to form an unsubstituted heterocycloalkyl. In some embodiments, two adjacent R²⁷ substituents are joined to form a substituted aryl. In some embodiments, two adjacent R²⁷ substituents are joined to form an unsubstituted aryl. In some embodiments, two adjacent R²⁷ substituents are joined to form a substituted heteroaryl. In some embodiments, two adjacent R²⁷ substituents are joined to form an unsubstituted heteroaryl.

In some embodiments, R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R² is substituted or unsubstituted alkyl. In some embodiments, R² is substituted or unsubstituted heteroalkyl. In some embodiments, R² is substituted or unsubstituted cycloalkyl. In some embodiments, R² is substituted or unsubstituted heterocycloalkyl. In some embodiments, R² is substituted or unsubstituted aryl. In some embodiments, R² is substituted or unsubstituted heteroaryl. In some embodiments, R² is unsubstituted alkyl. In some embodiments, R² is unsubstituted heteroalkyl. In some embodiments, R² is unsubstituted cycloalkyl. In some embodiments, R² is unsubstituted heterocycloalkyl. In some embodiments, R² is unsubstituted aryl. In some embodiments, R² is unsubstituted heteroaryl. In some embodiments, R² is substituted alkyl. In some embodiments, R² is substituted heteroalkyl. In some embodiments, R² is substituted cycloalkyl. In some embodiments, R² is substituted heterocycloalkyl. In some embodiments, R² is substituted aryl. In some embodiments, R² is substituted heteroaryl. In some embodiments, R² is R²³-substituted alkyl. In some embodiments, R² is R²³-substituted heteroalkyl. In some embodiments, R² is R²³-substituted cycloalkyl. In some embodiments, R² is R²³-substituted heterocycloalkyl. In some embodiments, R² is R²³-substituted aryl. In some embodiments, R² is R²³-substituted heteroaryl. In some embodiments, R² is substituted or unsubstituted phenyl. In some embodiments, R² is R²³-substituted or unsubstituted phenyl. In some embodiments, R² is substituted phenyl. In some embodiments, R² is unsubstituted phenyl. In some embodiments, R² is R²³-substituted phenyl. In some embodiments, R² is substituted or unsubstituted thienyl. In some embodiments, R² is R²³-substituted or unsubstituted thienyl. In some embodiments, R² is substituted thienyl. In some embodiments, R² is unsubstituted thienyl. In some embodiments, R² is R²³-substituted thienyl. In some embodiments, R² is substituted or unsubstituted 4,5,6,7-tetrahydrobenzo[b]thienyl. In some embodiments, R² is R²³-substituted or unsubstituted 4,5,6,7-tetrahydrobenzo[b]thienyl. In some embodiments, R² is substituted 4,5,6,7-tetrahydrobenzo[b]thienyl. In some embodiments, R² is unsubstituted 4,5,6,7-tetrahydrobenzo[b]thienyl. In some embodiments, R² is R²³-substituted 4,5,6,7-tetrahydrobenzo[b]thienyl. In some embodiments, R² is substituted or unsubstituted pyridyl. In some embodiments, R² is R²³-substituted or unsubstituted pyridyl. In some embodiments, R² is substituted pyridyl. In some embodiments, R² is unsubstituted pyridyl. In some embodiments, R² is R²³-substituted pyridyl. In some embodiments, R² is substituted or unsubstituted 1,3,4-thiadiazolyl. In some embodiments, R² is R²³-substituted or unsubstituted 1,3,4-thiadiazolyl. In some embodiments, R² is substituted 1,3,4-thiadiazolyl. In some embodiments, R² is unsubstituted 1,3,4-thiadiazolyl. In some embodiments, R² is R²³-substituted 1,3,4-thiadiazolyl. In some embodiments, R² is substituted or unsubstituted piperidinyl. In some embodiments, R² is R²³-substituted or unsubstituted piperidinyl. In some embodiments, R² is substituted piperidinyl. In some embodiments, R² is unsubstituted piperidinyl. In some embodiments, R² is R²³-substituted piperidinyl. In some embodiments, R² is substituted or unsubstituted piperazinyl. In some embodiments, R² is R²³-substituted or unsubstituted piperazinyl. In some embodiments, R² is substituted piperazinyl. In some embodiments, R² is unsubstituted piperazinyl. In some embodiments, R² is R²³-substituted piperazinyl. In some embodiments, R² is substituted or unsubstituted oxazolyl. In some embodiments, R² is R²³-substituted or unsubstituted oxazolyl. In some embodiments, R² is substituted oxazolyl. In some embodiments, R² is unsubstituted oxazolyl. In some embodiments, R² is R²³-substituted oxazolyl. In some embodiments, R² is substituted or unsubstituted thiazolyl. In some embodiments, R² is R²³-substituted or unsubstituted thiazolyl. In some embodiments, R² is substituted thiazolyl. In some embodiments, R² is unsubstituted thiazolyl. In some embodiments, R² is R²³-substituted thiazolyl. In some embodiments, R² is R²³-substituted or unsubstituted benzo[d]oxazolyl. In some embodiments, R² is R²³-substituted benzo[d]oxazolyl. In some embodiments, R² is unsubstituted benzo[d]oxazolyl.

In some embodiments, R² is R²³-substituted or unsubstituted imidazolyl. In some embodiments, R² is substituted imidazolyl. In some embodiments, R² is unsubstituted imidazolyl. In some embodiments, R² is R²³-substituted or unsubstituted 4H-1,2,4-triazolyl. In some embodiments, R² is substituted 4H-1,2,4-triazolyl. In some embodiments, R² is unsubstituted 4H-1,2,4-triazolyl. In some embodiments, R² is R²³-substituted or unsubstituted triazolyl. In some embodiments, R² is substituted triazolyl. In some embodiments, R² is unsubstituted triazolyl. In some embodiments, R² is substituted or unsubstituted naphthyl. In some embodiments, R² is substituted or unsubstituted furanyl. In some embodiments, R² is substituted or unsubstituted quinolinyl. In some embodiments, R² is unsubstituted naphthyl. In some embodiments, R² is unsubstituted furanyl. In some embodiments, R² is unsubstituted quinolinyl. In some embodiments, R² is substituted naphthyl. In some embodiments, R² is substituted furanyl. In some embodiments, R² is substituted quinolinyl. In some embodiments, R² is R²³-substituted naphthyl. In some embodiments, R² is R²³-substituted furanyl. In some embodiments, R² is R²³-substituted quinolinyl.

In some embodiments, R² is substituted with one R²³. In some embodiments, R² is substituted with two optionally different R²³. In some embodiments, R² is substituted with three optionally different R²³. In some embodiments, R² is substituted with four optionally different R²³. In some embodiments, two adjacent R²³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²³ substituents are joined to form a substituted cycloalkyl. In some embodiments, two adjacent R²³ substituents are joined to form an unsubstituted cycloalkyl. In some embodiments, two adjacent R²³ substituents are joined to form a substituted heterocycloalkyl. In some embodiments, two adjacent R²³ substituents are joined to form an unsubstituted heterocycloalkyl. In some embodiments, two adjacent R²³ substituents are joined to form a substituted aryl. In some embodiments, two adjacent R²³ substituents are joined to form an unsubstituted aryl. In some embodiments, two adjacent R²³ substituents are joined to form a substituted heteroaryl. In some embodiments, two adjacent R²³ substituents are joined to form an unsubstituted heteroaryl.

In some embodiments, R²³ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R²⁴-substituted or unsubstituted piperidinyl, R²⁴-substituted or unsubstituted piperazinyl, R²⁴-substituted or unsubstituted thiazolyl, R²⁴-substituted or unsubstituted oxazolyl, R²⁴-substituted or unsubstituted phenyl, R²⁴-substituted or unsubstituted thienyl, R²⁴-substituted or unsubstituted 4,5,6,7-tetrahydrobenzo[b]thienyl, R²⁴-substituted or unsubstituted pyridyl, or R²⁴-substituted or unsubstituted 1,3,4-thiadiazolyl. In some embodiments, R²³ is R²⁴-substituted or unsubstituted benzo[d]oxazolyl. In some embodiments, R²³ is R²⁴-substituted or unsubstituted imidazolyl. In some embodiments, R²³ is R²⁴-substituted imidazolyl. In some embodiments, R²³ is unsubstituted imidazolyl. In some embodiments, R²³ is R²⁴-substituted or unsubstituted 4H-1,2,4-triazolyl. In some embodiments, R²³ is R²⁴-substituted 4H-1,2,4-triazolyl. In some embodiments, R²³ is unsubstituted 4H-1,2,4-triazolyl. In some embodiments, R²³ is R²⁴-substituted or unsubstituted triazolyl. In some embodiments, R²³ is R²⁴-substituted triazolyl. In some embodiments, R²³ is unsubstituted triazolyl. In some embodiments, R²³ is independently R²⁴-substituted or unsubstituted alkyl, R²⁴-substituted or unsubstituted heteroalkyl, R²⁴-substituted or unsubstituted cycloalkyl, R²⁴ substituted or unsubstituted heterocycloalkyl, R²⁴-substituted or unsubstituted aryl, or R²⁴-substituted or unsubstituted heteroaryl. In some embodiments, R²³ is independently R²⁴-substituted alkyl. In some embodiments, R²³ is unsubstituted alkyl. In some embodiments, R²³ is R²⁴-substituted heteroalkyl. In some embodiments, R²³ is unsubstituted heteroalkyl. In some embodiments, R²³ is R²⁴-substituted cycloalkyl. In some embodiments, R²³ is unsubstituted cycloalkyl. In some embodiments, R²³ is R²⁴ substituted heterocycloalkyl. In some embodiments, R²³ is unsubstituted heterocycloalkyl. In some embodiments, R²³ is R²⁴-substituted aryl. In some embodiments, R²³ is unsubstituted aryl. In some embodiments, R²³ is R²⁴-substituted heteroaryl. In some embodiments, R²³ is unsubstituted heteroaryl. In some embodiments, R²³ is substituted with one R²⁴. In some embodiments, R²³ is substituted with two optionally different R²⁴. In some embodiments, R²³ is substituted with three optionally different R²⁴. In some embodiments, R²³ is substituted with four optionally different R²⁴.

In some embodiments, two adjacent R²⁴ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁴ substituents are joined to form a substituted cycloalkyl. In some embodiments, two adjacent R²⁴ substituents are joined to form an unsubstituted cycloalkyl. In some embodiments, two adjacent R²⁴ substituents are joined to form a substituted heterocycloalkyl. In some embodiments, two adjacent R²⁴ substituents are joined to form an unsubstituted heterocycloalkyl. In some embodiments, two adjacent R²⁴ substituents are joined to form a substituted aryl. In some embodiments, two adjacent R²⁴ substituents are joined to form an unsubstituted aryl. In some embodiments, two adjacent R²⁴ substituents are joined to form a substituted heteroaryl. In some embodiments, two adjacent R²⁴ substituents are joined to form an unsubstituted heteroaryl. In some embodiments, R²⁴ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R²⁴ is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, or R²⁵-substituted or unsubstituted heteroaryl. In some embodiments, R²⁴ is —CH₃. In some embodiments, R²⁴ is R²⁵-substituted or unsubstituted benzo[d]oxazolyl. In some embodiments, R²⁴ is R²⁵-substituted benzo[d]oxazolyl. In some embodiments, R²⁴ is unsubstituted benzo[d]oxazolyl. In some embodiments, R²⁴ is R²⁵-substituted or unsubstituted imidazolyl. In some embodiments, R²⁴ is R²⁵-substituted imidazolyl. In some embodiments, R²⁴ is unsubstituted imidazolyl. In some embodiments, R²⁴ is R²⁵-substituted or unsubstituted 4H-1,2,4-triazolyl. In some embodiments, R²⁴ is R²⁵-substituted 4H-1,2,4-triazolyl. In some embodiments, R²⁴ is unsubstituted 4H-1,2,4-triazolyl. In some embodiments, R²⁴ is R²⁵-substituted or unsubstituted triazolyl. In some embodiments, R²⁴ is R²⁵-substituted triazolyl. In some embodiments, R²⁴ is unsubstituted triazolyl. In some embodiments, R²⁴ is R²⁵-substituted or unsubstituted (C₁-C₄) alkyl. In some embodiments, R²⁴ is 2-propyl. In some embodiments, R²⁴ is methyl. In some embodiments, R²⁴ is ethyl. In some embodiments, R²⁴ is propyl. In some embodiments, R²⁴ is butyl. In some embodiments, R²⁴ is cyclopropyl. In some embodiments, R²⁴ is cyclobutyl.

In some embodiments, R⁴ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁴ is substituted or unsubstituted alkyl. In some embodiments, R⁴ is substituted or unsubstituted heteroalkyl. In some embodiments, R⁴ is substituted or unsubstituted cycloalkyl. In some embodiments, R⁴ is substituted or unsubstituted heterocycloalkyl. In some embodiments, R⁴ is substituted or unsubstituted aryl. In some embodiments, R⁴ is substituted or unsubstituted heteroaryl. In some embodiments, R⁴ is unsubstituted alkyl. In some embodiments, R⁴ is unsubstituted heteroalkyl. In some embodiments, R⁴ is unsubstituted cycloalkyl. In some embodiments, R⁴ is unsubstituted heterocycloalkyl. In some embodiments, R⁴ is unsubstituted aryl. In some embodiments, R⁴ is unsubstituted heteroaryl. In some embodiments, R⁴ is substituted alkyl. In some embodiments, R⁴ is substituted heteroalkyl. In some embodiments, R⁴ is substituted cycloalkyl. In some embodiments, R⁴ is substituted heterocycloalkyl. In some embodiments, R⁴ is substituted aryl. In some embodiments, R⁴ is substituted heteroaryl. In some embodiments, R⁴ is R²⁹-substituted alkyl. In some embodiments, R⁴ is R²⁹-substituted heteroalkyl. In some embodiments, R⁴ is R²⁹-substituted cycloalkyl. In some embodiments, R⁴ is R²⁹-substituted heterocycloalkyl. In some embodiments, R⁴ is R²⁹-substituted aryl. In some embodiments, R⁴ is R²⁹-substituted heteroaryl. In some embodiments, R⁴ is substituted or unsubstituted phenyl. In some embodiments, R⁴ is R²⁹-substituted or unsubstituted phenyl. In some embodiments, R⁴ is substituted phenyl. In some embodiments, R⁴ is unsubstituted phenyl. In some embodiments, R⁴ is R²⁹-substituted phenyl. In some embodiments, R⁴ is substituted or unsubstituted thienyl. In some embodiments, R⁴ is R²⁹-substituted or unsubstituted thienyl. In some embodiments, R⁴ is substituted thienyl. In some embodiments, R⁴ is unsubstituted thienyl. In some embodiments, R⁴ is R²⁹-substituted thienyl. In some embodiments, R⁴ is substituted or unsubstituted 4,5,6,7-tetrahydrobenzo[b]thienyl. In some embodiments, R⁴ is R²⁹-substituted or unsubstituted 4,5,6,7-tetrahydrobenzo[b]thienyl. In some embodiments, R⁴ is substituted 4,5,6,7-tetrahydrobenzo[b]thienyl. In some embodiments, R⁴ is unsubstituted 4,5,6,7-tetrahydrobenzo[b]thienyl. In some embodiments, R⁴ is R²⁹-substituted 4,5,6,7-tetrahydrobenzo[b]thienyl. In some embodiments, R⁴ is substituted or unsubstituted pyridyl. In some embodiments, R⁴ is R²⁹-substituted or unsubstituted pyridyl. In some embodiments, R⁴ is substituted pyridyl. In some embodiments, R⁴ is unsubstituted pyridyl. In some embodiments, R⁴ is R²⁹-substituted pyridyl. In some embodiments, R⁴ is substituted or unsubstituted 1,3,4-thiadiazolyl. In some embodiments, R⁴ is R²⁹-substituted or unsubstituted 1,3,4-thiadiazolyl. In some embodiments, R⁴ is substituted 1,3,4-thiadiazolyl. In some embodiments, R⁴ is unsubstituted 1,3,4-thiadiazolyl. In some embodiments, R⁴ is R²⁹-substituted 1,3,4-thiadiazolyl. In some embodiments, R⁴ is substituted or unsubstituted piperidinyl. In some embodiments, R⁴ is R²⁹-substituted or unsubstituted piperidinyl. In some embodiments, R⁴ is substituted piperidinyl. In some embodiments, R⁴ is unsubstituted piperidinyl. In some embodiments, R⁴ is R²⁹-substituted piperidinyl. In some embodiments, R⁴ is substituted or unsubstituted piperazinyl. In some embodiments, R⁴ is R²⁹-substituted or unsubstituted piperazinyl. In some embodiments, R⁴ is substituted piperazinyl. In some embodiments, R⁴ is unsubstituted piperazinyl. In some embodiments, R⁴ is R²⁹-substituted piperazinyl. In some embodiments, R⁴ is substituted or unsubstituted oxazolyl. In some embodiments, R⁴ is R²⁹-substituted or unsubstituted oxazolyl. In some embodiments, R⁴ is substituted oxazolyl. In some embodiments, R⁴ is unsubstituted oxazolyl. In some embodiments, R⁴ is R²⁹-substituted oxazolyl. In some embodiments, R⁴ is substituted or unsubstituted thiazolyl. In some embodiments, R⁴ is R²⁹-substituted or unsubstituted thiazolyl. In some embodiments, R⁴ is substituted thiazolyl. In some embodiments, R⁴ is unsubstituted thiazolyl. In some embodiments, R⁴ is R²⁹-substituted thiazolyl. In some embodiments, R⁴ is R²⁹-substituted or unsubstituted benzo[d]oxazolyl. In some embodiments, R⁴ is R²⁹-substituted benzo[d]oxazolyl. In some embodiments, R⁴ is unsubstituted benzo[d]oxazolyl.

In some embodiments, R⁴ is R²⁹-substituted or unsubstituted imidazolyl. In some embodiments, R⁴ is R²⁹-substituted imidazolyl. In some embodiments, R⁴ is unsubstituted imidazolyl. In some embodiments, R⁴ is R²⁹-substituted or unsubstituted 4H-1,2,4-triazolyl. In some embodiments, R⁴ is R²⁹-substituted 4H-1,2,4-triazolyl. In some embodiments, R⁴ is unsubstituted 4H-1,2,4-triazolyl. In some embodiments, R⁴ is R²⁹-substituted or unsubstituted triazolyl. In some embodiments, R⁴ is R²⁹-substituted triazolyl. In some embodiments, R⁴ is unsubstituted triazolyl. In some embodiments, R⁴ is substituted or unsubstituted naphthyl. In some embodiments, R⁴ is substituted or unsubstituted furanyl. In some embodiments, R⁴ is substituted or unsubstituted quinolinyl. In some embodiments, R⁴ is unsubstituted naphthyl. In some embodiments, R⁴ is unsubstituted furanyl. In some embodiments, R⁴ is unsubstituted quinolinyl. In some embodiments, R⁴ is substituted naphthyl. In some embodiments, R⁴ is substituted furanyl. In some embodiments, R⁴ is substituted quinolinyl. In some embodiments, R⁴ is R²⁹-substituted naphthyl. In some embodiments, R⁴ is R²⁹-substituted furanyl. In some embodiments, R⁴ is R²⁹-substituted quinolinyl.

In some embodiments, R⁴ is substituted with one R²⁹. In some embodiments, R⁴ is substituted with two optionally different R²⁹. In some embodiments, R⁴ is substituted with three optionally different R²⁹. In some embodiments, R⁴ is substituted with four optionally different R²⁹. In some embodiments, two adjacent R²⁹ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁹ substituents are joined to form a substituted cycloalkyl. In some embodiments, two adjacent R²⁹ substituents are joined to form an unsubstituted cycloalkyl. In some embodiments, two adjacent R²⁹ substituents are joined to form a substituted heterocycloalkyl. In some embodiments, two adjacent R²⁹ substituents are joined to form an unsubstituted heterocycloalkyl. In some embodiments, two adjacent R²⁹ substituents are joined to form a substituted aryl. In some embodiments, two adjacent R²⁹ substituents are joined to form an unsubstituted aryl. In some embodiments, two adjacent R²⁹ substituents are joined to form a substituted heteroaryl. In some embodiments, two adjacent R²⁹ substituents are joined to form an unsubstituted heteroaryl. In some embodiments, R²⁹ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R³⁰-substituted or unsubstituted piperidinyl, R³⁰-substituted or unsubstituted piperazinyl, R³⁰-substituted or unsubstituted thiazolyl, R³⁰-substituted or unsubstituted oxazolyl, R³⁰-substituted or unsubstituted phenyl, R³⁰-substituted or unsubstituted thienyl, R³⁰-substituted or unsubstituted 4,5,6,7-tetrahydrobenzo[b]thienyl, R³⁰-substituted or unsubstituted pyridyl, or R³⁰-substituted or unsubstituted 1,3,4-thiadiazolyl. In some embodiments, R²⁹ is R³⁰-substituted or unsubstituted benzo[d]oxazolyl. In some embodiments, R²⁹ is R³⁰-substituted or unsubstituted imidazolyl. In some embodiments, R²⁹ is R³⁰-substituted imidazolyl. In some embodiments, R²⁹ is unsubstituted imidazolyl. In some embodiments, R²⁹ is R³⁰-substituted or unsubstituted 4H-1,2,4-triazolyl. In some embodiments, R²⁹ is R³⁰-substituted 4H-1,2,4-triazolyl. In some embodiments, R²⁹ is unsubstituted 4H-1,2,4-triazolyl. In some embodiments, R²⁹ is R³⁰-substituted or unsubstituted triazolyl. In some embodiments, R²⁹ is R³⁰-substituted triazolyl. In some embodiments, R²⁹ is unsubstituted triazolyl. In some embodiments, R²⁹ is independently R³⁰-substituted or unsubstituted alkyl, R³⁰-substituted or unsubstituted heteroalkyl, R³⁰-substituted or unsubstituted cycloalkyl, R³⁰ substituted or unsubstituted heterocycloalkyl, R³⁰-substituted or unsubstituted aryl, or R³⁰-substituted or unsubstituted heteroaryl. In some embodiments, R²⁹ is independently R³⁰-substituted alkyl. In some embodiments, R²⁹ is unsubstituted alkyl. In some embodiments, R²⁹ is R³⁰-substituted heteroalkyl. In some embodiments, R²⁹ is unsubstituted heteroalkyl. In some embodiments, R²⁹ is R³⁰-substituted cycloalkyl. In some embodiments, R²⁹ is unsubstituted cycloalkyl. In some embodiments, R²⁹ is R³⁰ substituted heterocycloalkyl. In some embodiments, R²⁹ is unsubstituted heterocycloalkyl. In some embodiments, R²⁹ is R³⁰-substituted aryl. In some embodiments, R²⁹ is unsubstituted aryl. In some embodiments, R²⁹ is R³⁰-substituted heteroaryl. In some embodiments, R²⁹ is unsubstituted heteroaryl. In some embodiments, R²⁹ is substituted with one R³⁰. In some embodiments, R²⁹ is substituted with two optionally different R³⁰. In some embodiments, R²⁹ is substituted with three optionally different R³⁰. In some embodiments, R²⁹ is substituted with four optionally different R³⁰.

In some embodiments, two adjacent R³⁰ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R³⁰ substituents are joined to form a substituted cycloalkyl. In some embodiments, two adjacent R³⁰ substituents are joined to form an unsubstituted cycloalkyl. In some embodiments, two adjacent R³⁰ substituents are joined to form a substituted heterocycloalkyl. In some embodiments, two adjacent R³⁰ substituents are joined to form an unsubstituted heterocycloalkyl. In some embodiments, two adjacent R³⁰ substituents are joined to form a substituted aryl. In some embodiments, two adjacent R³⁰ substituents are joined to form an unsubstituted aryl. In some embodiments, two adjacent R³⁰ substituents are joined to form a substituted heteroaryl. In some embodiments, two adjacent R³⁰ substituents are joined to form an unsubstituted heteroaryl.

In some embodiments, R³⁰ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R³⁰ is R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl. In some embodiments, R³⁰ is CH₃. In some embodiments, R³⁰ is R³¹-substituted or unsubstituted benzo[d]oxazolyl. In some embodiments, R³⁰ is R³¹-substituted or unsubstituted imidazolyl. In some embodiments, R³⁰ is R³¹-substituted imidazolyl. In some embodiments, R³⁰ is unsubstituted imidazolyl. In some embodiments, R³⁰ is R³¹-substituted or unsubstituted 4H-1,2,4-triazolyl. In some embodiments, R³⁰ is R³¹-substituted 4H-1,2,4-triazolyl. In some embodiments, R³⁰ is unsubstituted 4H-1,2,4-triazolyl. In some embodiments, R³⁰ is R³¹-substituted or unsubstituted triazolyl. In some embodiments, R³⁰ is R³¹-substituted triazolyl. In some embodiments, R³⁰ is unsubstituted triazolyl. In some embodiments, R³⁰ is R³¹-substituted benzo[d]oxazolyl. In some embodiments, R³⁰ is unsubstituted benzo[d]oxazolyl. In some embodiments, R³⁰ is R³¹-substituted or unsubstituted (C₁-C₄) alkyl. In some embodiments, R³⁰ is 2-propyl. In some embodiments, R³⁰ is methyl. In some embodiments, R³⁰ is ethyl. In some embodiments, R³⁰ is propyl. In some embodiments, R³⁰ is butyl. In some embodiments, R³⁰ is cyclopropyl. In some embodiments, R³⁰ is cyclobutyl. In some embodiments, R³⁰ is substituted with one R³¹. In some embodiments, R³⁰ is substituted with two optionally different R³¹. In some embodiments, R³⁰ is substituted with three optionally different R³¹. In some embodiments, R³⁰ is substituted with four optionally different R³¹. In some embodiments, two adjacent R³¹ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R³¹ substituents are joined to form a substituted cycloalkyl. In some embodiments, two adjacent R³¹ substituents are joined to form an unsubstituted cycloalkyl. In some embodiments, two adjacent R³¹ substituents are joined to form a substituted heterocycloalkyl. In some embodiments, two adjacent R³¹ substituents are joined to form an unsubstituted heterocycloalkyl. In some embodiments, two adjacent R³¹ substituents are joined to form a substituted aryl. In some embodiments, two adjacent R³¹ substituents are joined to form an unsubstituted aryl. In some embodiments, two adjacent R³¹ substituents are joined to form a substituted heteroaryl. In some embodiments, two adjacent R³¹ substituents are joined to form an unsubstituted heteroaryl.

In some embodiments, R² and R⁴ may optionally be joined to form a substituted or unsubstituted heterocycloalkyl or substituted or unsubstituted heteroaryl. In some embodiments, R² and R⁴ are joined to form a substituted heterocycloalkyl. In some embodiments, R² and R⁴ are joined to form an unsubstituted heterocycloalkyl. In some embodiments, R² and R⁴ are joined to form a substituted aryl. In some embodiments, R² and R⁴ are joined to form a substituted heteroaryl.

In some embodiments, R² and R⁴ are joined to form an unsubstituted heteroaryl. In some embodiments, R² and R⁴ are joined to form an R²³-substituted heterocycloalkyl. In some embodiments, R² and R⁴ are joined to form an R²³-substituted heteroaryl. In some embodiments, R² and R⁴ are joined to form an unsubstituted heterocycloalkyl. In some embodiments, R² and R⁴ are joined to form an unsubstituted heteroaryl. In some embodiments, R² and R⁴ are joined to form a substituted or unsubstituted pyridyl. In some embodiments, R² and R⁴ are joined to form an R²³-substituted or unsubstituted pyridyl. In some embodiments, R² and R⁴ are joined to form a substituted pyridyl. In some embodiments, R² and R⁴ are joined to form an unsubstituted pyridyl. In some embodiments, R² and R⁴ are joined to form an R²³-substituted pyridyl. In some embodiments, R² and R⁴ are joined to form a substituted or unsubstituted 1,3,4-thiadiazolyl. In some embodiments, R² and R⁴ are joined to form an R²³-substituted or unsubstituted 1,3,4-thiadiazolyl. In some embodiments, R² and R⁴ are joined to form a substituted 1,3,4-thiadiazolyl. In some embodiments, R² and R⁴ are joined to form an unsubstituted 1,3,4-thiadiazolyl. In some embodiments, R² and R⁴ are joined to form an R²³-substituted 1,3,4-thiadiazolyl. In some embodiments, R² and R⁴ are joined to form a substituted or unsubstituted piperidinyl. In some embodiments, R² and R⁴ are joined to form an R²³-substituted or unsubstituted piperidinyl. In some embodiments, R² and R⁴ are joined to form a substituted piperidinyl. In some embodiments, R² and R⁴ are joined to form an unsubstituted piperidinyl. In some embodiments, R² and R⁴ are joined to form an R²³-substituted piperidinyl. In some embodiments, R² and R⁴ are joined to form a substituted or unsubstituted piperazinyl. In some embodiments, R² and R⁴ are joined to form an R²³-substituted or unsubstituted piperazinyl. In some embodiments, R² and R⁴ are joined to form a substituted piperazinyl. In some embodiments, R² and R⁴ are joined to form an unsubstituted piperazinyl. In some embodiments, R² and R⁴ are joined to form an R²³-substituted piperazinyl. In some embodiments, R² and R⁴ are joined to form a substituted or unsubstituted oxazolyl. In some embodiments, R² and R⁴ are joined to form an R²³-substituted or unsubstituted oxazolyl. In some embodiments, R² and R⁴ are joined to form a substituted oxazolyl. In some embodiments, R² and R⁴ are joined to form an unsubstituted oxazolyl. In some embodiments, R² and R⁴ are joined to form an R²³-substituted oxazolyl. In some embodiments, R² and R⁴ are joined to form a substituted or unsubstituted thiazolyl. In some embodiments, R² and R⁴ are joined to form an R²³-substituted or unsubstituted thiazolyl. In some embodiments, R² and R⁴ are joined to form a substituted thiazolyl. In some embodiments, R² and R⁴ are joined to form an unsubstituted thiazolyl. In some embodiments, R² and R⁴ are joined to form an R²³-substituted thiazolyl. In some embodiments, R² and R⁴ are joined to form an R²³-substituted or unsubstituted benzo[d]oxazolyl. In some embodiments, R² and R⁴ are joined to form an R²³-substituted benzo[d]oxazolyl. In some embodiments, R² and R⁴ are joined to form an unsubstituted benzo[d]oxazolyl. In some embodiments, R² and R⁴ are joined to form an R²³-substituted or unsubstituted imidazolyl. In some embodiments, R² and R⁴ are joined to form a substituted imidazolyl. In some embodiments, R² and R⁴ are joined to form an unsubstituted imidazolyl. In some embodiments, R² and R⁴ are joined to form an R²³-substituted or unsubstituted 4H-1,2,4-triazolyl. In some embodiments, R² and R⁴ are joined to form a substituted 4H-1,2,4-triazolyl. In some embodiments, R² and R⁴ are joined to form an unsubstituted 4H-1,2,4-triazolyl. In some embodiments, R² and R⁴ are joined to form an R²³-substituted or unsubstituted triazolyl. In some embodiments, R² and R⁴ are joined to form a substituted triazolyl. In some embodiments, R² and R⁴ are joined to form an unsubstituted triazolyl. In some embodiments, R² and R⁴ are joined to form a substituted or unsubstituted quinolinyl. In some embodiments, R² and R⁴ are joined to form an unsubstituted quinolinyl. In some embodiments, R² and R⁴ are joined to form a substituted quinolinyl. In some embodiments, R² and R⁴ are joined to form an R²³-substituted quinolinyl. In some embodiments, the ring formed by R² and R⁴ is substituted with one R²³. In some embodiments, the ring formed by R² and R⁴ is substituted with two optionally different R²³. In some embodiments, the ring formed by R² and R⁴ is substituted with three optionally different R²³. In some embodiments, the ring formed by R² and R⁴ is substituted with four optionally different R²³.

In some embodiments, two adjacent R²³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²³ substituents are joined to form a substituted cycloalkyl. In some embodiments, two adjacent R²³ substituents are joined to form an unsubstituted cycloalkyl. In some embodiments, two adjacent R²³ substituents are joined to form a substituted heterocycloalkyl. In some embodiments, two adjacent R²³ substituents are joined to form an unsubstituted heterocycloalkyl. In some embodiments, two adjacent R²³ substituents are joined to form a substituted aryl. In some embodiments, two adjacent R²³ substituents are joined to form an unsubstituted aryl. In some embodiments, two adjacent R²³ substituents are joined to form a substituted heteroaryl. In some embodiments, two adjacent R²³ substituents are joined to form an unsubstituted heteroaryl. In some embodiments, R²³ is independently oxo. In some embodiments, two adjacent R²³ substituents are joined to form an R²⁴-substituted cycloalkyl. In some embodiments, two adjacent R²³ substituents are joined to form an R²⁴-substituted heterocycloalkyl. In some embodiments, two adjacent R²³ substituents are joined to form an R²⁴-substituted aryl. In some embodiments, two adjacent R²³ substituents are joined to form an R²⁴-substituted heteroaryl. In some embodiments, two adjacent R²³ substituents are joined to form an R²⁴-substituted phenyl. In some embodiments, two adjacent R²³ substituents are joined to form an unsubstituted phenyl. In some embodiments, two adjacent R²³ substituents are joined to form an R²⁴-substituted pyridyl. In some embodiments, two adjacent R²³ substituents are joined to form an unsubstituted pyridyl. In some embodiments, two adjacent R²³ substituents are joined to form an R²⁴-substituted or unsubstituted piperidinyl, R²⁴-substituted or unsubstituted piperazinyl, R²⁴-substituted or unsubstituted thiazolyl, R²⁴-substituted or unsubstituted oxazolyl, R²⁴-substituted or unsubstituted phenyl, R²⁴-substituted or unsubstituted thienyl, R²⁴-substituted or unsubstituted 4,5,6,7-tetrahydrobenzo[b]thienyl, R²⁴-substituted or unsubstituted pyridyl, or R²⁴-substituted or unsubstituted 1,3,4-thiadiazolyl. In some embodiments, two adjacent R²³ substituents are joined to form an R²⁴-substituted or unsubstituted benzo[d]oxazolyl. In some embodiments, two adjacent R²³ substituents are joined to form an R²⁴-substituted or unsubstituted imidazolyl. In some embodiments, two adjacent R²³ substituents are joined to form an R²⁴-substituted imidazolyl. In some embodiments, two adjacent R²³ substituents are joined to form an unsubstituted imidazolyl. In some embodiments, two adjacent R²³ substituents are joined to form an R²⁴-substituted or unsubstituted 4H-1,2,4-triazolyl. In some embodiments, two adjacent R²³ substituents are joined to form an R²⁴-substituted 4H-1,2,4-triazolyl. In some embodiments, two adjacent R²³ substituents are joined to form an unsubstituted 4H-1,2,4-triazolyl. In some embodiments, two adjacent R²³ substituents are joined to form an R²⁴-substituted or unsubstituted triazolyl. In some embodiments, two adjacent R²³ substituents are joined to form an R²⁴-substituted triazolyl. In some embodiments, two adjacent R²³ substituents are joined to form an unsubstituted triazolyl.

In some embodiments, R² and R⁴ are joined to form an R²⁹-substituted heterocycloalkyl. In some embodiments, R² and R⁴ are joined to form an R²⁹-substituted heteroaryl. In some embodiments, R² and R⁴ are joined to form an unsubstituted heterocycloalkyl. In some embodiments, R² and R⁴ are joined to form an unsubstituted heteroaryl. In some embodiments, R² and R⁴ are joined to form a substituted or unsubstituted pyridyl. In some embodiments, R² and R⁴ are joined to form an R²⁹-substituted or unsubstituted pyridyl. In some embodiments, R² and R⁴ are joined to form a substituted pyridyl. In some embodiments, R² and R⁴ are joined to form an unsubstituted pyridyl. In some embodiments, R² and R⁴ are joined to form an R²⁹-substituted pyridyl. In some embodiments, R² and R⁴ are joined to form a substituted or unsubstituted 1,3,4-thiadiazolyl. In some embodiments, R² and R⁴ are joined to form an R²⁹-substituted or unsubstituted 1,3,4-thiadiazolyl. In some embodiments, R² and R⁴ are joined to form a substituted 1,3,4-thiadiazolyl. In some embodiments, R² and R⁴ are joined to form an unsubstituted 1,3,4-thiadiazolyl. In some embodiments, R² and R⁴ are joined to form an R²⁹-substituted 1,3,4-thiadiazolyl. In some embodiments, R² and R⁴ are joined to form a substituted or unsubstituted piperidinyl. In some embodiments, R² and R⁴ are joined to form an R²⁹-substituted or unsubstituted piperidinyl. In some embodiments, R² and R⁴ are joined to form a substituted piperidinyl. In some embodiments, R² and R⁴ are joined to form an unsubstituted piperidinyl. In some embodiments, R² and R⁴ are joined to form an R²⁹-substituted piperidinyl. In some embodiments, R² and R⁴ are joined to form a substituted or unsubstituted piperazinyl. In some embodiments, R² and R⁴ are joined to form an R²⁹-substituted or unsubstituted piperazinyl. In some embodiments, R² and R⁴ are joined to form a substituted piperazinyl. In some embodiments, R² and R⁴ are joined to form an unsubstituted piperazinyl. In some embodiments, R² and R⁴ are joined to form an R²⁹-substituted piperazinyl. In some embodiments, R² and R⁴ are joined to form a substituted or unsubstituted oxazolyl. In some embodiments, R² and R⁴ are joined to form an R²⁹-substituted or unsubstituted oxazolyl. In some embodiments, R² and R⁴ are joined to form a substituted oxazolyl. In some embodiments, R² and R⁴ are joined to form an unsubstituted oxazolyl. In some embodiments, R² and R⁴ are joined to form an R²⁹-substituted oxazolyl. In some embodiments, R² and R⁴ are joined to form a substituted or unsubstituted thiazolyl. In some embodiments, R² and R⁴ are joined to form an R²⁹-substituted or unsubstituted thiazolyl. In some embodiments, R² and R⁴ are joined to form a substituted thiazolyl. In some embodiments, R² and R⁴ are joined to form an unsubstituted thiazolyl. In some embodiments, R² and R⁴ are joined to form an R²⁹-substituted thiazolyl. In some embodiments, R² and R⁴ are joined to form an R²⁹-substituted or unsubstituted benzo[d]oxazolyl. In some embodiments, R² and R⁴ are joined to form an R²⁹-substituted benzo[d]oxazolyl. In some embodiments, R² and R⁴ are joined to form an unsubstituted benzo[d]oxazolyl. In some embodiments, R² and R⁴ are joined to form an R²⁹-substituted or unsubstituted imidazolyl. In some embodiments, R² and R⁴ are joined to form a substituted imidazolyl. In some embodiments, R² and R⁴ are joined to form an unsubstituted imidazolyl. In some embodiments, R² and R⁴ are joined to form an R²⁹-substituted or unsubstituted 4H-1,2,4-triazolyl. In some embodiments, R² and R⁴ are joined to form a substituted 4H-1,2,4-triazolyl. In some embodiments, R² and R⁴ are joined to form an unsubstituted 4H-1,2,4-triazolyl. In some embodiments, R² and R⁴ are joined to form an R²⁹-substituted or unsubstituted triazolyl. In some embodiments, R² and R⁴ are joined to form a substituted triazolyl. In some embodiments, R² and R⁴ are joined to form an unsubstituted triazolyl. In some embodiments, R² and R⁴ are joined to form a substituted or unsubstituted quinolinyl. In some embodiments, R² and R⁴ are joined to form an unsubstituted quinolinyl. In some embodiments, R² and R⁴ are joined to form a substituted quinolinyl. In some embodiments, R² and R⁴ are joined to form an R²⁹-substituted quinolinyl. In some embodiments, the ring formed by R² and R⁴ is substituted with one R²⁹. In some embodiments, the ring formed by R² and R⁴ is substituted with two optionally different R²⁹. In some embodiments, the ring formed by R² and R⁴ is substituted with three optionally different R²⁹. In some embodiments, the ring formed by R² and R⁴ is substituted with four optionally different R²⁹. In some embodiments, two adjacent R²⁹ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁹ substituents are joined to form a substituted cycloalkyl. In some embodiments, two adjacent R²⁹ substituents are joined to form an unsubstituted cycloalkyl. In some embodiments, two adjacent R²⁹ substituents are joined to form a substituted heterocycloalkyl. In some embodiments, two adjacent R²⁹ substituents are joined to form an unsubstituted heterocycloalkyl. In some embodiments, two adjacent R²⁹ substituents are joined to form a substituted aryl. In some embodiments, two adjacent R²⁹ substituents are joined to form an unsubstituted aryl. In some embodiments, two adjacent R²⁹ substituents are joined to form a substituted heteroaryl. In some embodiments, two adjacent R²⁹ substituents are joined to form an unsubstituted heteroaryl. In some embodiments, R²⁹ is independently oxo. In some embodiments, two adjacent R²⁹ substituents are joined to form an R³⁰-substituted cycloalkyl. In some embodiments, two adjacent R²⁹ substituents are joined to form an R³⁰-substituted heterocycloalkyl. In some embodiments, two adjacent R²⁹ substituents are joined to form an R³⁰-substituted aryl. In some embodiments, two adjacent R²⁹ substituents are joined to form an R³⁰-substituted heteroaryl. In some embodiments, two adjacent R²⁹ substituents are joined to form an R³⁰-substituted phenyl. In some embodiments, two adjacent R²⁹ substituents are joined to form an unsubstituted phenyl. In some embodiments, two adjacent R²⁹ substituents are joined to form an R³⁰-substituted pyridyl. In some embodiments, two adjacent R²⁹ substituents are joined to form an unsubstituted pyridyl. In some embodiments, two adjacent R²⁹ substituents are joined to form an R³⁰-substituted or unsubstituted piperidinyl, R³⁰-substituted or unsubstituted piperazinyl, R³⁰-substituted or unsubstituted thiazolyl, R³⁰-substituted or unsubstituted oxazolyl, R³⁰-substituted or unsubstituted phenyl, R³⁰-substituted or unsubstituted thienyl, R³⁰-substituted or unsubstituted 4,5,6,7-tetrahydrobenzo[b]thienyl, R³⁰-substituted or unsubstituted pyridyl, or R³⁰-substituted or unsubstituted 1,3,4-thiadiazolyl. In some embodiments, two adjacent R²⁹ substituents are joined to form an R³⁰-substituted or unsubstituted benzo[d]oxazolyl. In some embodiments, two adjacent R²⁹ substituents are joined to form an R³⁰-substituted or unsubstituted imidazolyl. In some embodiments, two adjacent R²⁹ substituents are joined to form an R³⁰-substituted imidazolyl. In some embodiments, two adjacent R²⁹ substituents are joined to form an unsubstituted imidazolyl. In some embodiments, two adjacent R²⁹ substituents are joined to form an R³⁰-substituted or unsubstituted 4H-1,2,4-triazolyl. In some embodiments, two adjacent R²⁹ substituents are joined to form an R³⁰-substituted 4H-1,2,4-triazolyl. In some embodiments, two adjacent R²⁹ substituents are joined to form an unsubstituted 4H-1,2,4-triazolyl. In some embodiments, two adjacent R²⁹ substituents are joined to form an R³⁰-substituted or unsubstituted triazolyl. In some embodiments, two adjacent R²⁹ substituents are joined to form an R³⁰-substituted triazolyl. In some embodiments, two adjacent R²⁹ substituents are joined to form an unsubstituted triazolyl. Where each R⁷, R⁸, R⁹, and R¹⁰, X, n, v, and m is different, they may be referred to, for example, as R^{7c}, R^{7d}, R^{7e}, R^{7f}, R^{7g}, R^{7h}, R⁷ⁱ, R^{8c}, R^{8d}, R^{8e}, R^{8f}, R^{8g}, R^{8h}, R⁸ⁱ, R^{9c}, R^{9d}, R^{9e}, R^{9f}, R^{9g}, R^{9h}, R⁹ⁱ, R^{10c}, R^{10d}, R^{10e}, R^{10f}, R^{10g}, R^{10h}, R¹⁰ⁱ, X^{c}, X^{d}, X^{e}, X^{f}, X^{g}, X^{h}, Xⁱ, n^{c}, n^{d}, n^{e}, n^{f}, n^{g}, n^{h}, nⁱ, v^{c}, v^{d}, v^{e}, v^{f}, v^{g}, V^{h}, vⁱ, m^{c}, m^{d}, m^{e}, m^{f}, m^{g}, m^{h}, mⁱ, and so on, wherein each R^{7c}, R^{7d}, R^{7e}, R^{7f}, R^{7g}, R^{7h}, R⁷ⁱ is defined the same as R⁷, each R^{8c}, R^{8d}, R^{8e}, R^{8f}, R^{8g}, R^{8h}, R⁸ⁱ is defined the same as R⁸, each R^{9c}, R^{9d}, R^{9e}, R^{9f}, R^{9g}, R^{9h}, R⁹ⁱ is defined the same as R⁹, each R^{10c}, R^{10d}, R^{10e}, R^{10f}, R^{10g}, R^{10h}, R¹⁰ⁱ is defined the same as R¹⁰, each X^{c}, X^{d}, X^{e}, X^{f}, X^{g}, X^{h}, Xⁱ is defined the same as X, each n^{c}, n^{d}, n^{e}, n^{f}, n^{g}, n^{h}, nⁱ is defined the same as n, each v^{c}, v^{d}, v^{f}, v^{g}, v^{h}, vⁱ is defined the same as v, each m^{c}, m^{d}, m^{e}, m^{f}, m^{g}, m^{h}, mⁱ is defined the same as m. In some embodiments, R² is defined by R^{7c}, R^{8c}, R^{9c}, R^{10c}, X^{c}, n^{c}, v^{c}, and m^{c}. In some embodiments, R³ is defined by R^{7d}, R^{8d}, R^{9d}, R^{10d}, X^{d}, n^{d}, v^{d}, and m^{d}. In some embodiments, R⁴ is defined by R^{7e}, R^{8e}, R^{9e}, R^{10e}, X^{e}, n^{e}, v^{e}, and m^{e}. In some embodiments, R⁶ is defined by R^{7f}, R^{8f}, R^{9f}, R^{10f}, X^{f}, n^{f}, v^{f}, and m^{f}. In some embodiments, R¹ is defined by R^{7g}, R^{8g}, R^{9g}, R^{10g}, X^{g}, n^{g}, v^{g}, and m^{g}. In some embodiments, R^{1A} is defined by R^{7h}, R^{8h}, R^{9h}, R^{10h}, X^{h}, V^{h}, and m^{h}. In some embodiments, R^{1B} is defined by R⁷ⁱ, R⁸ⁱ, R⁹ⁱ, R¹⁰ⁱ, Xⁱ, nⁱ, vⁱ, and mⁱ. Where c, d, e, f, g, h, and i denote substituents of R², R³, R⁴, R⁶, R¹¹, R^{1A}, and R^{1B} respectively.

In some embodiments, the compound having formula (XI) is a compound having the formula:

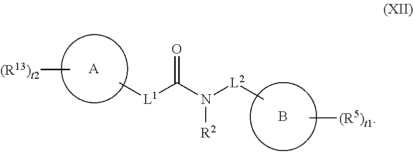

Ring B, L¹, L², R^{1A}, R^{1B}, X, X^{a}, X^{b}, m, n, p, q, r, v, m1, v1, n1, t1, R², R³, R⁴, R⁵, R⁷, R⁸, R⁹, R¹⁰, R^{7b}, R^{8b}, R^{9b}, R^{10b}, R¹³, R¹⁴, R¹⁵, R¹⁶, and R¹⁷ are as described herein (e.g. formula (I) to (XI), including embodiments).

Ring A is substituted or unsubstituted cycloalkyl, substituted or unsubstituted heterocycloalkyl, substituted or unsubstituted aryl, or substituted or unsubstituted heteroaryl.

The symbol t2 is an integer from 0 to 8. Where each R¹⁴, R¹⁵, R¹⁶, and R¹⁷, X^{a}, r, p, and q is different, they may be referred to, for example, as R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R¹³ is defined by R^{14c}, R^{15c}, R^{16c}, R^{17c}, X^{ac}, r^{c}, p^{c}, and q^{c}. In some embodiments, R¹³ is defined by R^{14d}, R^{15d}, R^{16d}, R^{17d}, X^{ad}, r^{d}, p^{d}, and q^{d}. In some embodiments, R¹³ is defined by R^{14e}, R^{15e}, R^{16e}, and R^{17e}, X^{ae}, r^{e}, p^{e}, and q^{e}. In some embodiments, R¹³ is defined by R^{14f}, R^{15f}, R^{16f}, and R^{17f}, X^{af}, r^{f}, p^{f}, and q^{f}. In some embodiments, R¹³ is defined by R^{14g}, R^{15g}, R^{16g}, and R^{17g}, X^{ag}, r^{g}, p^{g}, and q^{g}. Where c, d, e, f, and g denote substituents of different R¹³ respectively. Where each R^{7b}, R^{8b}, R^{9b}, and R^{10b}, X^{b}, n1, v1, and m1 is different, they may be referred to, for example, as R^{7bc}, R^{7bc}, R^{7be}, R^{7bf}, R^{7bg}, R^{8bc}, R^{8bd}, R^{8be}, R^{8bf}, R^{8bg}, R^{9bc}, R^{9bd}, R^{9be}, R^{9bf}, R_{9bg}, R^{10bc}, R^{10bc}, R^{10be}, R^{10bf}, R^{10bg}, X^{bc}, X^{bd}, X^{be}, X^{bf}, X^{bg}, n1^{c}, n1^{d}, n1^{e}, n1^{f}, n1^{g}, v1^{c}, v1^{d}, v1^{e}, v1^{f}, v1^{g}, m1^{c}, m1^{c}, m1^{d}, m1^{e}, m1^{f}, m1^{g}, and so on, wherein each R^{7bc}, R^{7bd}, R^{7be}, R^{7bf}, R^{7bg} is defined the same as R^{7b}, each R^{8bc}, R^{8bd}, R^{8be}, R^{8bf}, R^{8bg} is defined the same as R^{8b}, each R^{9bc}, R^{9bd}, R^{9be}, R^{9bf}, R^{9bg} is defined the same as R^{9b}, each R^{10bc}, R^{10bd}, R^{10be}, R^{10bf}, R^{10bg} is defined the same as R^{10b}, each X^{bc}, X^{bd}, X^{be}, X^{bf}, X^{bg} is defined the same as X^{b}, each n1^{c}, n1^{d}, n1^{e}, n1^{f}, n1^{g} is defined the same as n1, each v1^{c}, v1^{d}, v1^{e}, v1^{f}, v1^{g} is defined the same as v1, each m1^{c}, m1^{d}, m1^{f}, m1^{g} is defined the same as m1. In some embodiments, R⁵ is defined by R^{7bc}, R^{8bc}, R^{9bc}, R^{10bc}, X^{bc}, n1^{c}, v1^{c}, and m1^{c}. In some embodiments, R⁵ is defined by R^{7bd}, R^{8bd}, R^{9bd}, R^{10bd}, X^{bd}, n1^{d}, v1^{d}, m1^{d}. In some embodiments, R⁵ is defined by R^{7be}, R^{8be}, R^{9be}, R^{10be}, X^{be}, n1^{e}, v1^{e}, and m1^{e}. In some embodiments, R⁵ is defined by R^{7bf}, R^{8bf}, R^{9bf}, R^{10bf}, X^{bf}, n1^{f}, v1^{f} and m1^{f}. In some embodiments, R⁵ is defined by R^{7bg}, R^{8bg}, R^{9bg}, R^{10bg}, X^{bg}, n1^{g}, v1^{g}, and m1^{g}. Where c, d, e, f, and g denote substituents of different R⁵ respectively.

In some embodiments, the compound having formula XII is a compound having the formula:

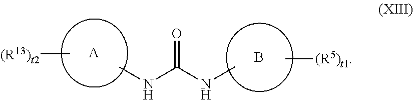

Ring A, Ring B, X^{a}, X^{b}, p, q, r, m1, v1, n1, t1, t2, R⁵, R^{7b}, R^{8b}, R^{9b}, R^{10b}, R¹³, R¹⁴, R¹⁵, R¹⁶, and R¹⁷ are as described herein (e.g. formula (I) to (XII), including embodiments).

In some embodiments, L¹, L², R^{1A}, R^{1B}, X, m, n, v, R², R³, R⁴, R⁷, R⁸, R⁹, and R¹⁰, are as described in the paragraphs below in a compound of formula (XIII). In some embodiments, these values are included in any other formula described herein. In some embodiments, ring A is substituted or unsubstituted cycloalkyl, substituted or unsubstituted heterocycloalkyl, substituted or unsubstituted aryl, or substituted or unsubstituted heteroaryl. In some embodiments, ring B is substituted or unsubstituted cycloalkyl, substituted or unsubstituted heterocycloalkyl, substituted or unsubstituted aryl, or substituted or unsubstituted heteroaryl. In some embodiments, R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, R^{7b}, R^{8b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹³ is independently hydrogen, halogen, —CX^{a}, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, ml, p, q, and v1 are independently an integer from 1 to 2. In some embodiments, the symbols n, n1, and r are independently an integer from 0 to 4. In some embodiments, the symbol t1 is an integer from 0 to 8. In some embodiments, the symbol t2 is an integer from 0 to 8. In some embodiments, the symbols Xa and X^{b} are independently —Cl, —Br, —I, or —F. In some embodiments, the symbol m1 is 1. In some embodiments, the symbol m1 is 2. In some embodiments, the symbol v1 is 1. In some embodiments, the symbol v1 is 2. In some embodiments, p is 1. In some embodiments, p is 2. In some embodiments, q is 1. In some embodiments, q is 2. In some embodiments, the symbol n is 0. In some embodiments, the symbol n is 1. In some embodiments, the symbol n is 2. In some embodiments, the symbol n is 3. In some embodiments, the symbol n is 4. In some embodiments, the symbol n1 is 0. In some embodiments, the symbol n1 is 1. In some embodiments, the symbol n1 is 2. In some embodiments, the symbol n1 is 3. In some embodiments, the symbol n1 is 4. In some embodiments, r is 0. In some embodiments, r is 1. In some embodiments, r is 2. In some embodiments, r is 3. In some embodiments, r is 4. In some embodiments, t1 is 0. In some embodiments, t1 is 1. In some embodiments, t1 is 2. In some embodiments, t1 is 3. In some embodiments, t1 is 4. In some embodiments, t1 is 5. In some embodiments, t1 is 6. In some embodiments, t1 is 7. In some embodiments, t1 is 8. In some embodiments, t2 is 0. In some embodiments, t2 is 1. In some embodiments, t2 is 2. In some embodiments, t2 is 3. In some embodiments, t2 is 4. In some embodiments, t2 is 5. In some embodiments, t2 is 6. In some embodiments, t2 is 7. In some embodiments, t2 is 8. In some embodiments, X′ is —Cl. In some embodiments, X^{a} is —Br. In some embodiments, X^{a} is —I. In some embodiments, X^{a} is —F. In some embodiments, X^{b} is —Cl. In some embodiments, X^{b} is —Br. In some embodiments, X^{b} is —I. In some embodiments, X^{b} is —F.

In some embodiments, ring A is substituted or unsubstituted cycloalkyl. In some embodiments, ring A is substituted or unsubstituted heterocycloalkyl. In some embodiments, ring A is substituted or unsubstituted aryl. In some embodiments, ring A is substituted or unsubstituted heteroaryl. In some embodiments, ring A is unsubstituted cycloalkyl. In some embodiments, ring A is unsubstituted heterocycloalkyl. In some embodiments, ring A is unsubstituted aryl. In some embodiments, ring A is unsubstituted heteroaryl. In some embodiments, ring A is substituted cycloalkyl. In some embodiments, ring A is substituted heterocycloalkyl. In some embodiments, ring A is substituted aryl. In some embodiments, ring A is substituted heteroaryl. In some embodiments, ring A is substituted phenyl. In some embodiments, ring A is unsubstituted phenyl. In some embodiments, R¹³ is independently halogen, —CX^{a}₃, —CN, —N(O)_{q}, —NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹³ is independently halogen, —CF₃, —CN, —N(O)₂, —NH₂, or —OH. In some embodiments, R¹³ is independently unsubstituted alkyl, unsubstituted heteroalkyl, unsubstituted cycloalkyl, unsubstituted heterocycloalkyl, unsubstituted aryl, or unsubstituted heteroaryl. In some embodiments, R¹³ is —Cl. In some embodiments, R¹³ is —Br. In some embodiments, R¹³ is —I. In some embodiments, R¹³ is —F. In some embodiments, R¹³ is independently two different halogens. In some embodiments, R¹³ is —CF₃. In some embodiments, R¹³ is —CN. In some embodiments, R¹³ is —N(O)₂. In some embodiments, R¹³ is —NH₂. In some embodiments, R¹³ is —OH. In some embodiments, ring B is substituted or unsubstituted cycloalkyl. In some embodiments, ring B is substituted or unsubstituted heterocycloalkyl. In some embodiments, ring B is substituted or unsubstituted aryl. In some embodiments, ring B is substituted or unsubstituted heteroaryl. In some embodiments, ring B is unsubstituted cycloalkyl. In some embodiments, ring B is unsubstituted heterocycloalkyl. In some embodiments, ring B is unsubstituted aryl. In some embodiments, ring B is unsubstituted heteroaryl. In some embodiments, ring B is substituted cycloalkyl. In some embodiments, ring B is substituted heterocycloalkyl. In some embodiments, ring B is substituted aryl. In some embodiments, ring B is substituted heteroaryl. In some embodiments, ring B is substituted phenyl. In some embodiments, ring B is unsubstituted phenyl. In some embodiments, R⁵ is independently halogen, —CX^{b}₃, —CN, —N(O)ₘ₁, —NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁵ is independently halogen, —CF₃, —CN, —N(O)₂, —NH₂, or —OH. In some embodiments, R⁵ is independently unsubstituted alkyl, unsubstituted heteroalkyl, unsubstituted cycloalkyl, unsubstituted heterocycloalkyl, unsubstituted aryl, or unsubstituted heteroaryl. In some embodiments, R⁵ is —Cl. In some embodiments, R⁵ is —Br. In some embodiments, R⁵ is —I. In some embodiments, R⁵ is —F. In some embodiments, R⁵ is independently two different halogens. In some embodiments, R⁵ is —CF₃. In some embodiments, R⁵ is —CN. In some embodiments, R⁵ is —N(O)₂. In some embodiments, R⁵ is —NH₂. In some embodiments, R⁵ is —OH.

In some embodiments, multiple R^{aa} substituents may be present on the same compound, wherein R^{aa} is any R group described herein, and each R^{aa} may be different (e.g. SR^{bb} and —OR^{bb}, if both such groups are possible R^{aa} groups) and each R^{bb} group within each R^{aa} may be different even if the R^{bb} groups have the same identifying number (e.g. SR^{bb} and —OR^{bb}, wherein the R^{bb} of SR^{bb} is for example substituted alkyl or a CH₂CH₂OH group and the R^{bb} of OR^{bb} is for example, unsubstituted heteroaryl or unsubstituted indolyl). Where each R^{7b}, R^{8b}, R^{9b}, and R^{10b}, X^{b}, n1, v1, and m1 is different, they may be referred to, for example, as R^{7bc}, R^{7bd}, R^{7be}, R^{7bf}, R^{bg}, R^{8bc}, R^{8bd}, R^{8be}, R^{8bf}, R^{8bg}, R^{9bc}, R^{9bd}, R^{9be}, R^{9bf}, R_{9bg}, R^{10bc}, R^{10bc}, R^{10be}, R^{10bf}, R^{10bg}, X^{bc}, X^{bd}, X^{be}, X^{bf}, X^{bg}, n1^{c}, n1^{d}, n1^{e}, n1^{f}, n1^{g}, v1^{c}, v1^{d}, v1^{e}, v1^{f}, v1^{g}, m1^{c}, m1^{c}, m1^{d}, m1^{e}, m1^{f}, m1^{g}, and so on, wherein each R^{7bc}, R^{7bd}, R^{7be}, R^{7bf}, R^{7bg} is defined the same as R^{7b}, each R^{8bc}, R^{8bd}, R^{8be}, R^{8bf}, R^{8bg} is defined the same as R^{8b}, each R^{9bc}, R^{9bd}, R^{9be}, R^{9bf}, R^{9bg} is defined the same as R^{9b}, each R^{10bc}, R^{10bd}, R^{10be}, R^{10bf}, R^{10bg} is defined the same as R^{10b}, each X^{bc}, X^{bd}, X^{be}, X^{bf}, X^{bg} is defined the same as X^{b}, each n1^{c}, n1^{d}, n1^{e}, n1^{f}, n1^{g} is defined the same as n1, each v1^{c}, v1^{d}, v1^{e}, v1^{f}, v1^{g} is defined the same as v1, each m1^{c}, m1^{d}, m1^{f}, m1^{g} is defined the same as m1. In some embodiments, R⁵ is defined by R^{7bc}, R^{8bc}, R^{9bc}, R^{10bc}, X^{bc}, n1^{c}, v1^{c}, and m1^{c}. In some embodiments, R⁵ is defined by R^{7bd}, R^{8bd}, R^{9bd}, R^{10bd}, X^{bd}, n1^{d}, v1^{d}, m1^{d}. In some embodiments, R⁵ is defined by R^{7be}, R^{8be}, R^{9be}, R^{10be}, X^{be}, n1^{e}, v1^{e}, and m1^{e}. In some embodiments, R⁵ is defined by R^{7bf}, R^{8bf}, R^{9bf}, R^{10bf}, X^{bf}, n1^{f}, v1^{f} and m1^{f}. In some embodiments, R⁵ is defined by R^{7bg}, R^{8bg}, R^{9bg}, R^{10bg}, X^{bg}, n1^{g}, v1^{g}, and m1^{g}. Where c, d, e, f, and g denote substituents of different R⁵ respectively. Where each R¹⁴, R¹⁵, R¹⁶, and R¹⁷, X^{a}, r, p, and q is different, they may be referred to, for example, as R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R¹³ is defined by R^{14c}, R^{15c}, R^{16c}, R^{17c}, X^{ac}, r^{c}, p^{c}, and q^{c}. In some embodiments, R¹³ is defined by R^{14d}, R^{15d}, R^{16d}, R^{17d}, X^{ad}, r^{d}, p^{d}, and q^{d}. In some embodiments, R¹³ is defined by R^{14e}, R^{15e}, R^{16e}, and R^{17e}, X^{ae}, r^{e}, p^{e}, and q^{e}. In some embodiments, R¹³ is defined by R^{14f}, R^{15f}, R^{16f}, and R^{17f}, X^{af}, r^{f}, p^{f}, and q^{f}. In some embodiments, R¹³ is defined by R^{14g}, R^{15g}, R^{16g}, and R^{17g}, X^{ag}, r^{g}, p^{g}, and q^{g}. Where c, d, e, f, and g denote substituents of different R¹³ respectively.

In some embodiments, the compound having formula XII is a compound having the formula:

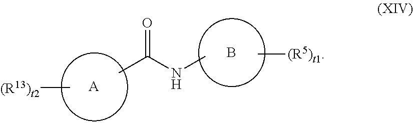

Ring A, Ring B, X^{a}, X^{b}, p, q, r, m1, v1, n1, t1, t2, R⁵, R^{7b}, R^{8b}, R^{9b}, R^{10b}, R¹³, R¹⁴, R¹⁵, R¹⁶, and R¹⁷ are as described herein (e.g. formula (I) to (XIII), including embodiments).

In some embodiments, the compound having formula XII is a compound having the formula:

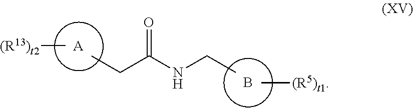

Ring A, Ring B, X^{a}, X^{b}, p, q, r, m1, v1, n1, t1, t2, R⁵, R^{7b}, R^{8b}, R^{9b}, R^{10b}, R¹³, R¹⁴, R¹⁵, R¹⁶, and R¹⁷ are as described herein (e.g. formula (I) to (XIV), including embodiments).

In some embodiments, the compound having formula XI is a compound having the formula:

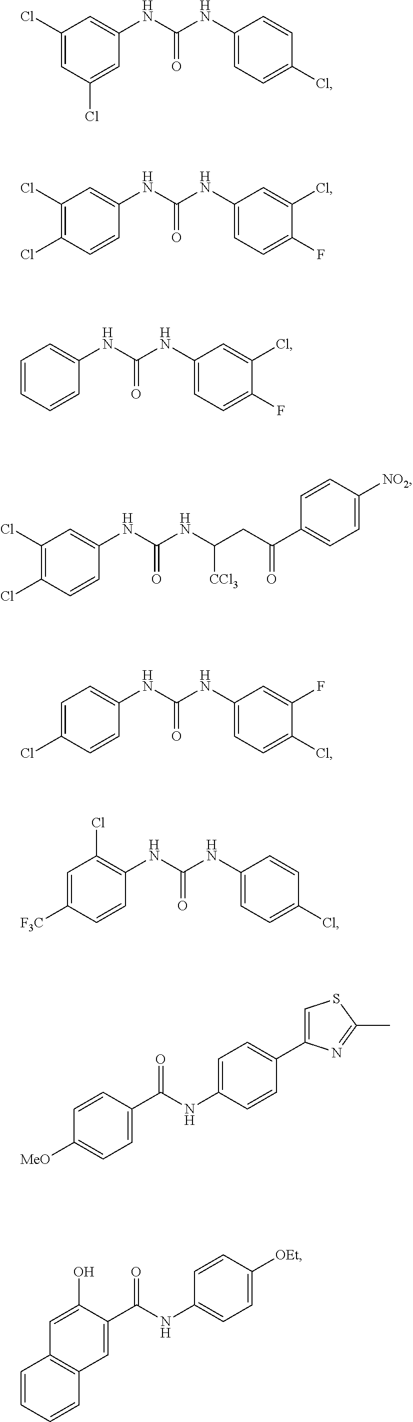

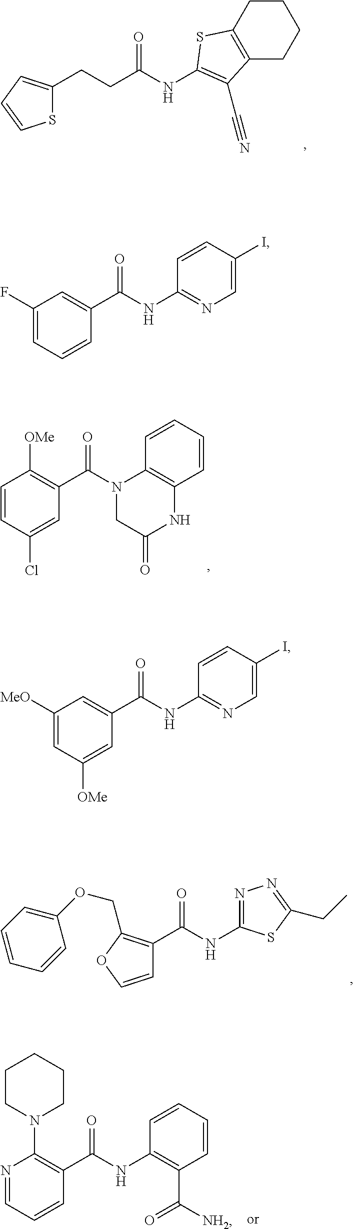

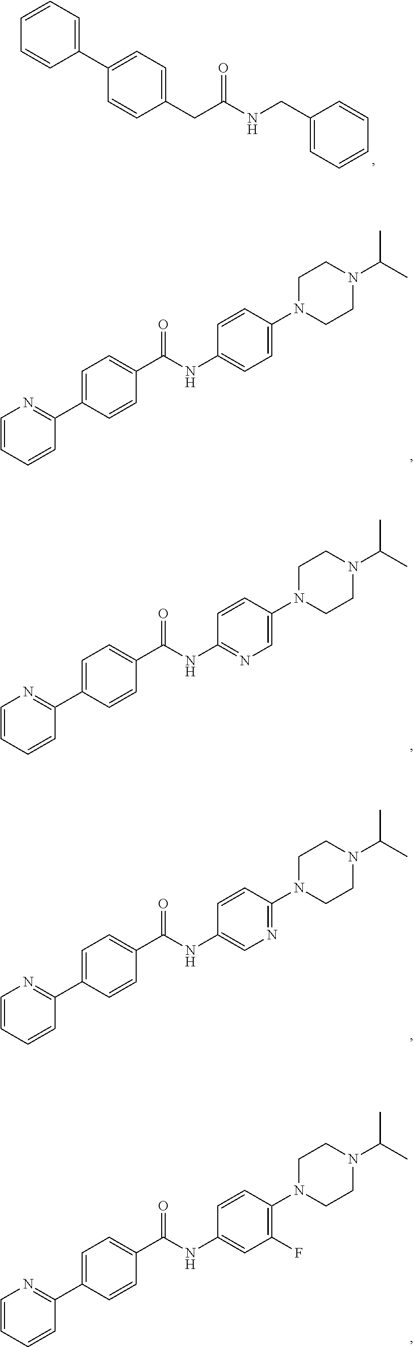 [SMILES: O=C(Cc1ccc(-c2ccccc2)cc1)NCc1ccccc1 ; CC(C)N1CCN(c2ccc(NC(=O)c3ccc(-c4ccccn4)cc3)cc2)CC1 ; CC(C)N1CCN(c2ccc(NC(=O)c3ccc(-c4ccccn4)cc3)nc2)CC1 ; CC(C)N1CCN(c2ccc(NC(=O)c3ccc(-c4ccccn4)cc3)cn2)CC1 ; CC(C)N1CCN(c2ccc(NC(=O)c3ccc(-c4ccccn4)cc3)cc2F)CC1]

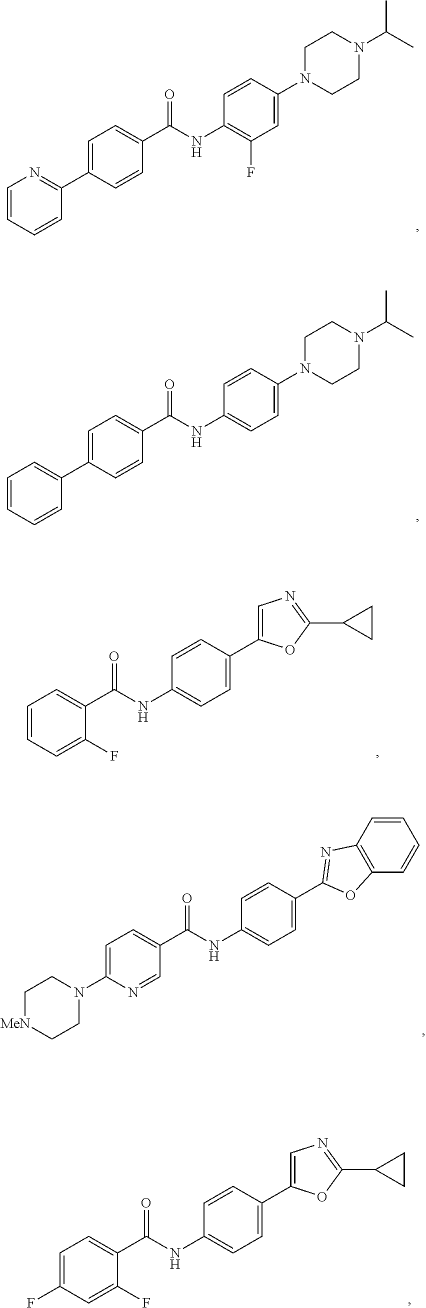 [SMILES: CC(C)N1CCN(c2ccc(NC(=O)c3ccc(-c4ccccn4)cc3)c(F)c2)CC1 ; CC(C)N1CCN(c2ccc(NC(=O)c3ccc(-c4ccccc4)cc3)cc2)CC1 ; O=C(Nc1ccc(-c2cnc(C3CC3)o2)cc1)c1ccccc1F ; CN1CCN(c2ccc(C(=O)Nc3ccc(-c4nc5ccccc5o4)cc3)cn2)CC1 ; O=C(Nc1ccc(-c2cnc(C3CC3)o2)cc1)c1ccc(F)cc1F]

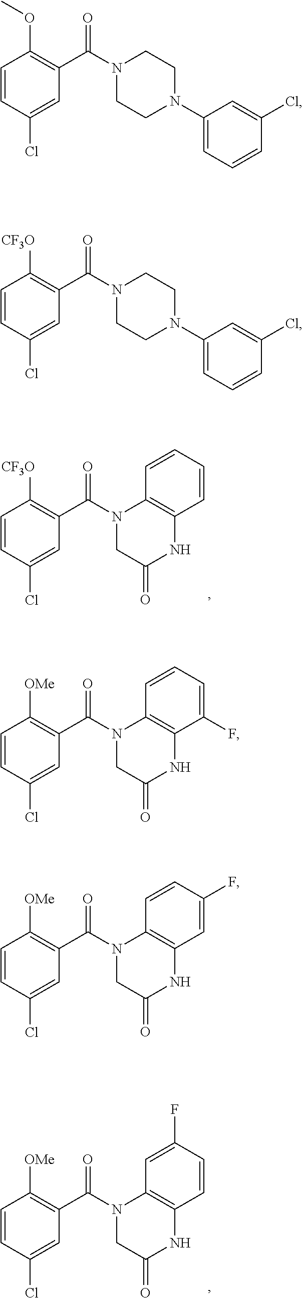

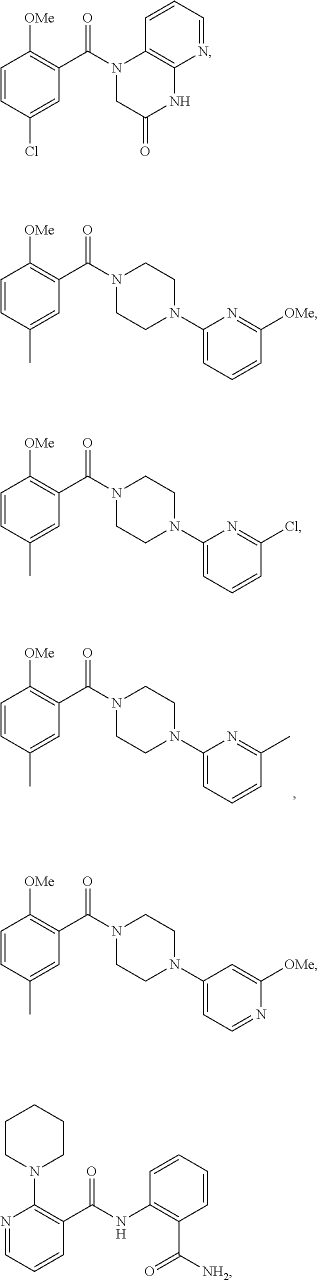 [SMILES: COc1ccc(Cl)cc1C(=O)N1CC(=O)Nc2ncccc21 ; COc1cccc(N2CCN(C(=O)c3cc(C)ccc3OC)CC2)n1 ; COc1ccc(C)cc1C(=O)N1CCN(c2cccc(Cl)n2)CC1 ; COc1ccc(C)cc1C(=O)N1CCN(c2cccc(C)n2)CC1 ; COc1cc(N2CCN(C(=O)c3cc(C)ccc3OC)CC2)ccn1 ; NC(=O)c1ccccc1NC(=O)c1cccnc1N1CCCCC1]

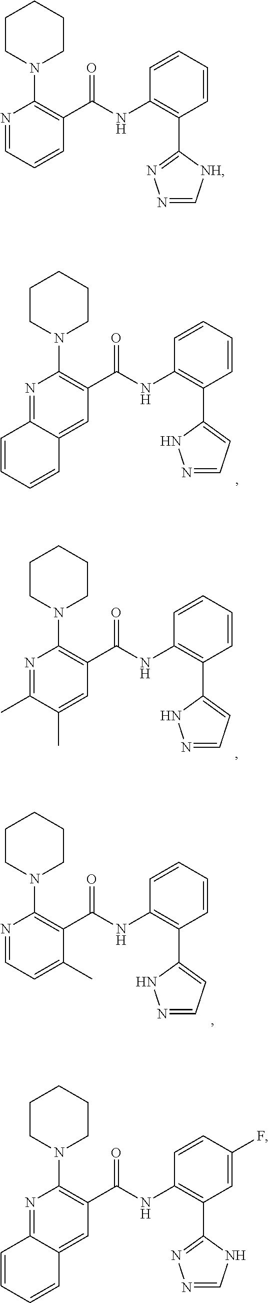 [SMILES: O=C(Nc1ccccc1-c1nnc[nH]1)c1cccnc1N1CCCCC1 ; O=C(Nc1ccccc1-c1ccn[nH]1)c1cc2ccccc2nc1N1CCCCC1 ; Cc1cc(C(=O)Nc2ccccc2-c2ccn[nH]2)c(N2CCCCC2)nc1C ; Cc1ccnc(N2CCCCC2)c1C(=O)Nc1ccccc1-c1ccn[nH]1 ; O=C(Nc1ccc(F)cc1-c1nnc[nH]1)c1cc2ccccc2nc1N1CCCCC1]

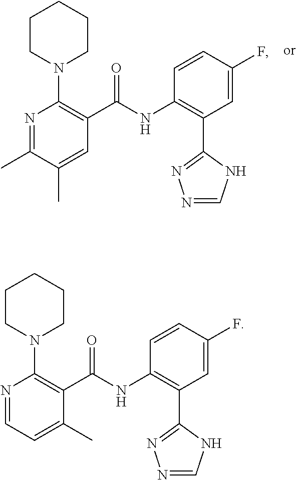 [SMILES: Cc1cc(C(=O)Nc2ccc(F)cc2-c2nnc[nH]2)c(N2CCCCC2)nc1C ; "or" ; Cc1ccnc(N2CCCCC2)c1C(=O)Nc1ccc(F)cc1-c1nnc[nH]1]

In another aspect, is a compound having the formula:

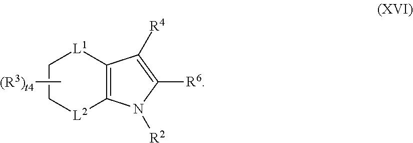

L¹, L², X, R^{1A}, R^{1B}, R², R³, R⁴, R⁶, R⁷, R⁸, R⁹, R¹⁰, m, n and v are as described herein (e.g. formula (I) to (XV), including embodiments).

In some embodiments, two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, R² and R⁶ substituents may optionally be joined to form a substituted or unsubstituted heterocycloalkyl or substituted or unsubstituted heteroaryl. In some embodiments, R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl. The symbol t4 is an integer from 0 to 2.

In some embodiments the compound having formula XVI is a compound having the formula:

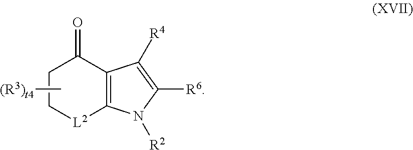

L², R^{1B}, R², R³, R⁴, R⁶, R⁷, R⁸, R⁹, R¹⁰, X, M, v, n, t4 are as described herein (e.g. formula (I) to (XVI), including embodiments).

In some embodiments, two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, R² and R⁶ substituents may optionally be joined to form a substituted or unsubstituted heterocycloalkyl or substituted or unsubstituted heteroaryl. In some embodiments, R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl.

In some embodiments, the compound having formula XVI is a compound having the formula:

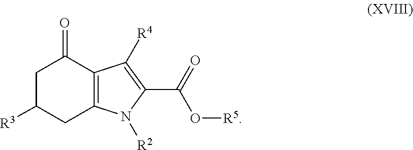

R², R³, R⁴, R⁵, R⁷, R⁸, R⁹, R¹⁰, R^{7b}, R^{8b}, R^{9b}, R^{10b}, X, X^{b}, m, v, n, m1, v1, n1, are as described herein (e.g. formula (I) to (XVII), including embodiments). In some embodiments, two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl.

In some embodiments, the compound having formula XVI is a compound having the formula:

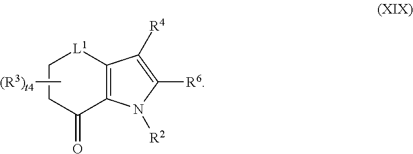

L¹, R^{1A}, R², R³, R⁴, R⁵, R⁶, R⁷, R⁸, R⁹, R¹⁰, R^{7b}, R^{8b}, R^{9b}, R^{10b}, X, X^{b}, m, m1, v, v1, n, n1, and t4 are as described herein (e.g. formula (I) to (XVIII), including embodiments).

In some embodiments, the compound having formula XVI is a compound having the formula:

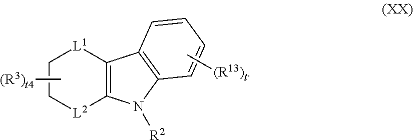

L¹, L², R^{1A}, R^{1B}, R², R³, R⁴, R⁵, R⁶, R⁷, R⁸, R⁹, R¹⁰, R^{7b}, R^{8b}, R^{9b}, R^{10b}, R¹³, R¹⁴, R¹⁵, R¹⁶, R¹⁷, X, X^{a}, X^{b}, m, m1, v, v1, n, n1, p, q, r, t, and t4 are as described herein (e.g. formula (I) to (XIX), including embodiments).

In some embodiments, two adjacent R¹³ substituents may optionally be joined to form a substituted or unsubstitued cycloalkyl, substituted or unsubstituted heterocycloalkyl, substituted or unsubstituted aryl, or substituted or unsubstituted heteroaryl.

In some ebodiments, the compound having formula XVI is a compound having the formula:

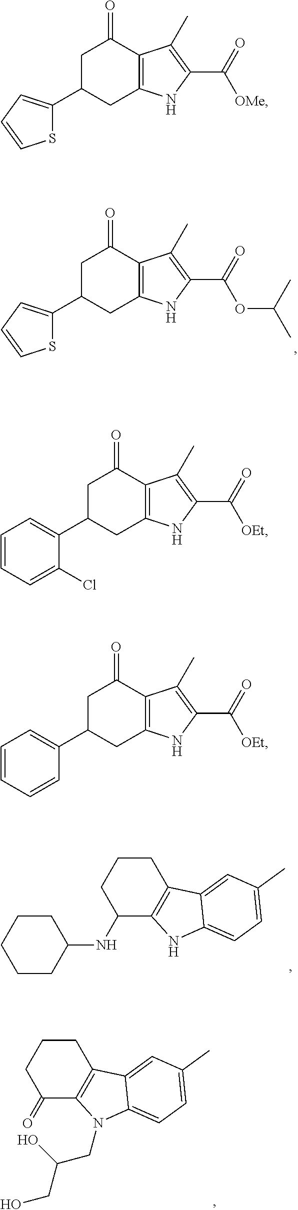 [SMILES: COC(=O)c1[nH]c2c(c1C)C(=O)CC(c1cccs1)C2 ; Cc1c(C(=O)OC(C)C)[nH]c2c1C(=O)CC(c1cccs1)C2 ; CCOC(=O)c1[nH]c2c(c1C)C(=O)CC(c1ccccc1Cl)C2 ; CCOC(=O)c1[nH]c2c(c1C)C(=O)CC(c1ccccc1)C2 ; Cc1ccc2[nH]c3c(c2c1)CCCC3NC1CCCCC1 ; Cc1ccc2c(c1)c1c(n2CC(O)CO)C(=O)CCC1]

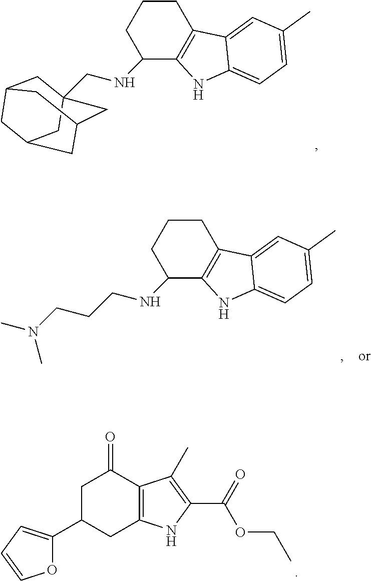

In another aspect is a compound having the formula:

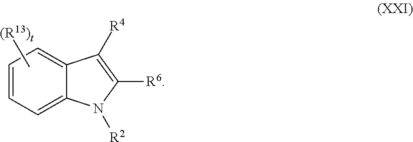

R², R⁴, R⁶, R⁷, R⁸, R⁹, R¹⁰, R¹³, R¹⁴, R¹⁵, R¹⁶, R¹⁷, X, X^{a}, m, v, n, p, q, r, and t are as described herein (e.g. formula (I) to (XX), including embodiments).

In some embodiments, R² and R⁶ substituents may optionally be joined to form a substituted or unsubstituted heterocycloalkyl or substituted or unsubstituted heteroaryl. In some embodiments, R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl

In some embodiments, the compound having formula XXI is a compound having the formula:

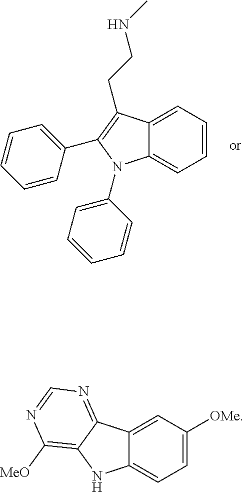 [SMILES: CNCCc1c(-c2ccccc2)n(-c2ccccc2)c2ccccc12 ; COc1ccc2[nH]c3c(OC)ncnc3c2c1]

In another aspect is a compound having the formula:

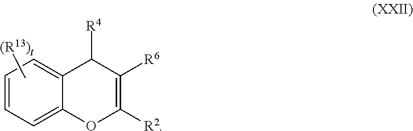

R², R⁴, R⁶, R⁷, R⁸, R⁹, R¹⁰, R¹³, R¹⁴, R¹⁵, R¹⁶, R¹⁷, X, X^{a}, m, v, n, p, q, r, and t are as described herein (e.g. formula (I) to (XXI), including embodiments).

In some embodiments, R² and R⁶ substituents may optionally be joined to form a substituted or unsubstituted heterocycloalkyl or substituted or unsubstituted heteroaryl. In some embodiments, R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl

In some embodiments the compound having formula XXII is a compound having the formula:

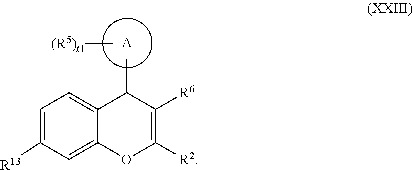

Ring A, R², R⁶, R⁷, R⁸, R⁹, R¹⁰, R¹³, R¹⁴, R¹⁵, R¹⁶, R¹⁷, X, X^{a}, m, p, v, n, r, q, and t1 are as described herein (e.g. formula (I) to (XXII), including embodiments).

In some embodiments, two adjacent R⁵ substituents may optionally be joined to form a substituted or unsubstitued cycloalkyl, substituted or unsubstituted heterocycloalkyl, substituted or unsubstituted aryl, or substituted or unsubstituted heteroaryl.

In some embodiments the compound having formula XXII is a compound having the formula:

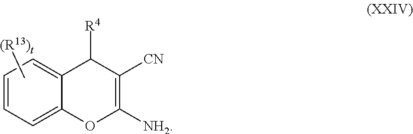

R⁴, R⁷, R⁸, R⁹, R¹⁰, R¹³, R¹⁴, R¹⁵, R¹⁶, R¹⁷, X^{a}, X, m, p, v, n, r, q, and t are as described herein (e.g. formula (I) to (XXIII), including embodiments).

In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl.

In some embodiments the compound having formula XXII is a compound having the formula:

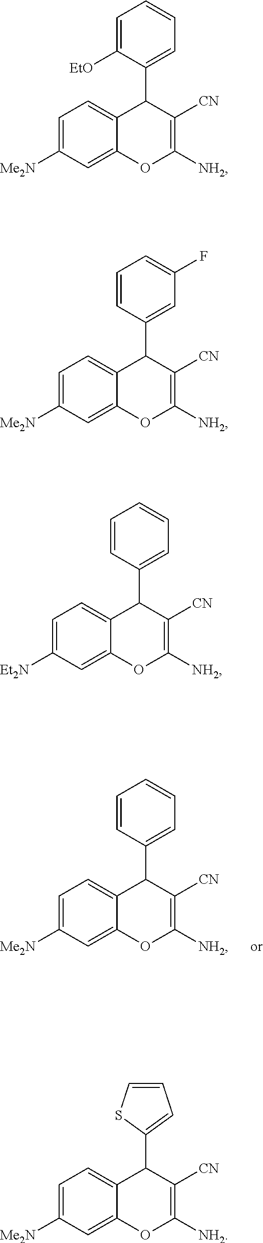

In another aspect is a compound having the formula:

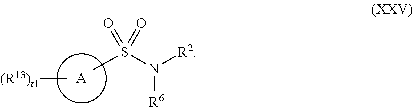

Ring A, R², R⁶, R⁷, R⁸, R⁹, R¹⁰, R¹³, R¹⁴, R¹⁵, R¹⁶, R¹⁷, X, X^{a}, m, p, v, n, r, q, and t1 are as described herein (e.g. formula (I) to (XXIV), including embodiments).

In some embodiments, R² and R⁶ substituents may optionally be joined to form a substituted or unsubstituted heterocycloalkyl or substituted or unsubstituted heteroaryl. In some embodiments, two adjacent R¹³ substituents may optionally be joined to form a substituted or unsubstitued cycloalkyl, substituted or unsubstituted heterocycloalkyl, substituted or unsubstituted aryl, or substituted or unsubstituted heteroaryl.

In some embodiments, Ring A, R², R⁶, R⁷, R⁸, R⁹, R¹⁰, R¹³, R¹⁴, R¹⁵, R¹⁶, R¹⁷, X, X^{a}, m, p, v, n, r, q, and t1, are as described in the paragraphs below in a compound of formula (XXV). In some embodiments, these values are included in any other formula described herein. In some embodiments, Ring A is a substituted or unsubstituted aryl. In some embodiments, Ring A is a substituted or unsubstituted heteroaryl. In some embodiments, Ring A is an unsubstituted aryl. In some embodiments, Ring A is an unsubstituted heteroaryl. In some embodiments, Ring A is a substituted or unsubstituted phenyl. In some embodiments, Ring A is an unsubstituted phenyl. In some embodiments, Ring A is a substituted or unsubstituted naphthyl. In some embodiments, Ring A is an unsubstituted naphthyl. In some embodiments, Ring A is a substituted or unsubstituted dihydrobenzofuran. In some embodiments, Ring A is an unsubstituted dihydrobenzofuran. In some embodiments, Ring A is a substituted or unsubstituted benzofuran. In some embodiments, Ring A is an unsubstituted benzofuran. In some embodiments, Ring A is a substituted or unsubstituted fused ring heterocycloalkyl-aryl. In some embodiments, Ring A is an unsubstituted fused ring heterocycloalkyl-aryl. In some embodiments, Ring A is a substituted or unsubstituted pyridinyl. In some embodiments, Ring A is an unsubstituted pyridinyl. In some embodiments, R² and R⁶ are independently hydrogen, halogen, —CX₃, —CN, —SO₂Cl, —SOₙR¹⁰, SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R² and R⁶ substituents may optionally be joined to form a substituted or unsubstituted heterocycloalkyl or substituted or unsubstituted heteroaryl. In some embodiments, R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R² is hydrogen. In some embodiments, R⁶ is hydrogen. In some embodiments, R² is substituted or unsubstituted aryl or heteroaryl. In some embodiments, R² is substituted or unsubstituted aryl. In some embodiments, R² is substituted or unsubstituted heteroaryl. In some embodiments, R² is unsubstituted aryl. In some embodiments, R² is substituted aryl. In some embodiments, R² is substituted heteroaryl. In some embodiments, R² is unsubstituted heteroaryl. In some embodiments, R² is R²³-substituted aryl. In some embodiments, R²³ is R²⁴-substituted or unsubstituted alkyl. In some embodiments, R²³ is R²⁴-substituted or unsubstituted heteroalkyl. In some embodiments, R²³ is unsubstituted alkyl. In some embodiments, R²³ is unsubstituted (C₁-C₄) alkyl. In some embodiments, R²³ is unsubstituted heteroalkyl. In some embodiments, R²³ is unsubstituted alkoxy. In some embodiments, R²³ is unsubstituted (C₁-C₄) alkoxy. In some embodiments, R²³ is methyl. In some embodiments, R²³ is OCH₃. In some embodiments, R²³ is halogen. In some embodiments, R²³ is —Cl. In some embodiments, R² is substituted with two R²³ substituents, wherein the two R²³ substituents are different. In some embodiments, R² is substituted with two R²³ substituents, wherein the two R²³ substituents are identical. In some embodiments, R² is substituted with two R²³ substituents, wherein the two R²³ substituents are selected from unsubstituted alkyl, unsubstituted heteroalkyl, and halogen. In some embodiments, R² is substituted with three R²³ substituents, wherein the three R²³ substituents are different. In some embodiments, R² is substituted with three R²³ substituents, wherein the three R²³ substituents are identical. In some embodiments, R⁶ is substituted or unsubstituted aryl or heteroaryl. In some embodiments, R⁶ is substituted or unsubstituted aryl. In some embodiments, R⁶ is substituted or unsubstituted heteroaryl. In some embodiments, R⁶ is unsubstituted aryl. In some embodiments, R⁶ is substituted aryl. In some embodiments, R⁶ is substituted heteroaryl. In some embodiments, R⁶ is unsubstituted heteroaryl. In some embodiments, R⁶ is R³⁵-substituted aryl. In some embodiments, R³⁵ is R³⁶-substituted or unsubstituted alkyl. In some embodiments, R³⁵ is R³⁶-substituted or unsubstituted heteroalkyl. In some embodiments, R³⁵ is unsubstituted alkyl. In some embodiments, R³⁵ is unsubstituted (C₁-C₄) alkyl. In some embodiments, R³⁵ is unsubstituted heteroalkyl. In some embodiments, R³⁵ is unsubstituted alkoxy. In some embodiments, R³⁵ is unsubstituted (C₁-C₄) alkoxy. In some embodiments, R³⁵ is methyl. In some embodiments, R³⁵ is OCH₃. In some embodiments, R³⁵ is halogen. In some embodiments, R³⁵ is —Cl. In some embodiments, R⁶ is substituted with two R³⁵ substituents, wherein the two R³⁵ substituents are different. In some embodiments, R⁶ is substituted with two R³⁵ substituents, wherein the two R³⁵ substituents are identical. In some embodiments, R⁶ is substituted with two R³⁵ substituents, wherein the two R³⁵ substituents are selected from unsubstituted alkyl, unsubstituted heteroalkyl, and halogen. In some embodiments, R⁶ is substituted with three R³⁵ substituents, wherein the three R³⁵ substituents are different. In some embodiments, R⁶ is substituted with three R³⁵ substituents, wherein the three R³⁵ substituents are identical. In some embodiments, R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, the symbols m, p, q, and v are independently an integer from 1 to 2. In some embodiments, the symbols n and r are independently an integer from 0 to 4. In some embodiments, the symbol t1 is an integer from 0 to 8. In some embodiments, the symbols X and X^{a} are independently —Cl, —Br, —I, or —F. In some embodiments, R¹³ is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹³ is a substituted or unsubstituted alkyl. In some embodiments, R¹³ is an unsubstituted alkyl. In some embodiments, R¹³ is a methyl. In some embodiments, R¹³ is a substituted or unsubstituted heteroalkyl. In some embodiments, R¹³ is an unsubstituted heteroalkyl. In some embodiments, R¹³ is a OCH₃. In some embodiments, R¹³ is a substituted or unsubstituted heteroaryl. In some embodiments, R¹³ is an unsubstituted heteroaryl. In some embodiments, R¹³ is a substituted or unsubstituted pyridinyl. In some embodiments, R¹³ is a pyrrolidin-2-one. In some embodiments, R¹³ is a pyrrolidinyl-2-one. In some embodiments, two adjacent R¹³ substituents are joined to form a substituted or unsubstituted heterocycloalkyl. In some embodiments, two adjacent R¹³ substituents are joined to form an unsubstituted heterocycloalkyl. In some embodiments, two adjacent R¹³ substituents are joined to form a substituted or unsubstituted dihydrofuran. In some embodiments, two adjacent R¹³ substituents are joined to form an unsubstituted dihydrofuran. In some embodiments, the symbol t1 is 1. In some embodiments, the symbol t1 is 2. In some embodiments, the symbol t1 is 3. In some embodiments, the symbol t1 is 4. In some embodiments, the symbol t1 is 5. In some embodiments, the symbol t1 is 6. In some embodiments, the symbol t1 is 7. In some embodiments, the symbol t1 is 8. Where each R⁷, R⁸, R⁹, and R¹⁰, X, n, v, and m is different, they may be referred to, for example, as R^{7c}, R^{7d}, R^{7e}, R^{7f}, R^{7g}, R^{7h}, R⁷ⁱ, R^{8c}, R^{8d}, R^{8e}, R^{8f}, R^{8g}, R^{8h}, R⁸ⁱ, R^{9c}, R^{9d}, R^{9e}, R^{9f}, R^{9g}, R^{9h}, R⁹ⁱ, R^{10c}, R^{10d}, R^{10e}, R^{10f}, R^{10g}, R^{10h}, R¹⁰ⁱ, X^{c}, X^{d}, X^{e}, X^{f}, X^{g}, X^{h}, Xⁱ, n^{c}, n^{d}, n^{e}, n^{f}, n^{g}, n^{h}, nⁱ, v^{c}, v^{d}, v^{e}, v^{f}, v^{g}, V^{h}, vⁱ, m^{c}, m^{d}, m^{e}, m^{f}, m^{g}, m^{h}, mⁱ, and so on, wherein each R^{7c}, R^{7d}, R^{7e}, R^{7f}, R^{7g}, R^{7h}, R⁷ⁱ is defined the same as R⁷, each R^{8c}, R^{8d}, R^{8e}, R^{8f}, R^{8g}, R^{8h}, R⁸ⁱ is defined the same as R⁸, each R^{9c}, R^{9d}, R^{9e}, R^{9f}, R^{9g}, R^{9h}, R⁹ⁱ is defined the same as R⁹, each R^{10c}, R^{10d}, R^{10e}, R^{10f}, R^{10g}, R^{10h}, R¹⁰ⁱ is defined the same as R¹⁰, each X^{c}, X^{d}, X^{e}, X^{f}, X^{g}, X^{h}, Xⁱ is defined the same as X, each n^{c}, n^{d}, n^{e}, n^{f}, n^{g}, n^{h}, nⁱ is defined the same as n, each v^{c}, v^{d}, v^{f}, v^{g}, v^{h}, vⁱ is defined the same as v, each m^{c}, m^{d}, m^{e}, m^{f}, m^{g}, m^{h}, mⁱ is defined the same as m. In some embodiments, R² is defined by R^{7c}, R^{8c}, R^{9c}, R^{10c}, X^{c}, n^{c}, v^{c}, and m^{c}. In some embodiments, R³ is defined by R^{7d}, R^{8d}, R^{9d}, R^{10d}, X^{d}, n^{d}, v^{d}, and m^{d}. In some embodiments, R⁴ is defined by R^{7e}, R^{8e}, R^{9e}, R^{10e}, X^{e}, n^{e}, v^{e}, and m^{e}. In some embodiments, R⁶ is defined by R^{7f}, R^{8f}, R^{9f}, R^{10f}, X^{f}, n^{f}, v^{f}, and m^{f}. In some embodiments, R¹ is defined by R^{7g}, R^{8g}, R^{9g}, R^{10g}, X^{g}, n^{g}, v^{g}, and m^{g}. In some embodiments, R^{1A} is defined by R^{7h}, R^{8h}, R^{9h}, R^{10h}, X^{h}, V^{h}, and m^{h}. In some embodiments, R^{1B} is defined by R⁷ⁱ, R⁸ⁱ, R⁹ⁱ, R¹⁰ⁱ, Xⁱ, nⁱ, vⁱ, and mⁱ. Where c, d, e, f, g, h, and i denote substituents of R², R³, R⁴, R⁶, R¹¹, R^{1A}, and R^{1B} respectively. Where each R¹⁴, R¹⁵, R¹⁶, and R¹⁷, X^{a}, r, p, and q is different, they may be referred to, for example, as R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R¹³ is defined by R^{14c}, R^{15c}, R^{16c}, R^{17c}, X^{ac}, r^{c}, p^{c}, and q^{c}. In some embodiments, R¹³ is defined by R^{14d}, R^{15d}, R^{16d}, R^{17d}, X^{ad}, r^{d}, p^{d}, and q^{d}. In some embodiments, R¹³ is defined by R^{14e}, R^{15e}, R^{16e}, and

R^{17e}, X^{ae}, r^{e}, p^{e}, and q^{e}. In some embodiments, R¹³ is defined by R^{14f}, R^{15f}, R^{16f}, and R^{17f}, X^{af}, r^{f}, p^{f}, and q^{f}. In some embodiments, R¹³ is defined by R^{14g}, R^{15g}, R^{16g}, and R^{17g}, X^{ag}, r^{g}, p^{g}, and q^{g}. Where c, d, e, f, and g denote substituents of different R¹³ respectively.

In some embodiments the compound having formula XXV is a compound having the formula:

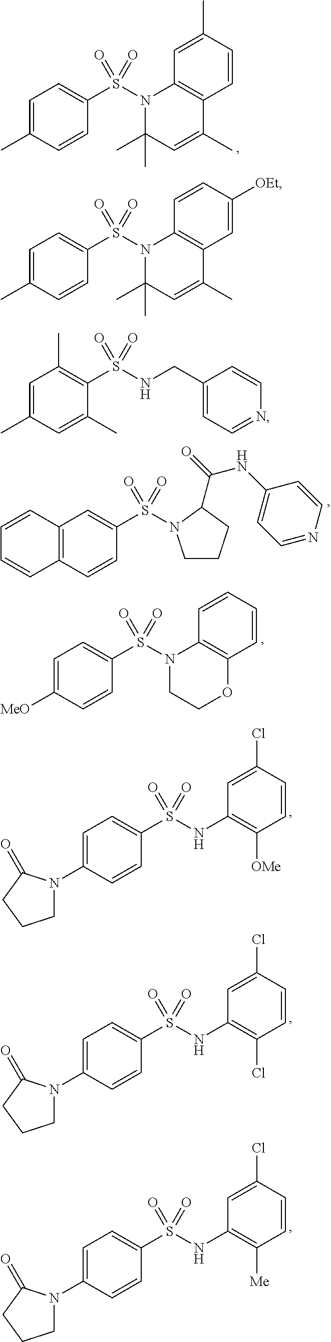

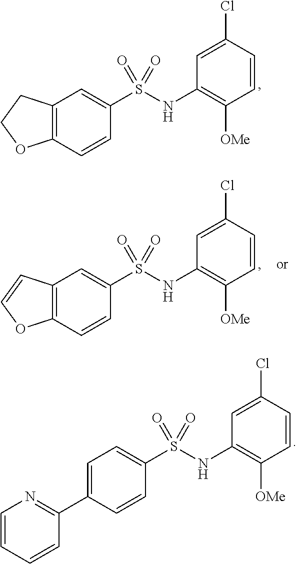

In another aspect is a compound having the formula:

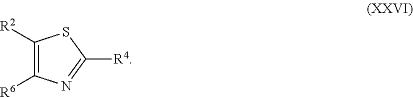

R², R⁴, R⁶, R⁷, R⁸, R⁹, R¹⁰, X, m, v, and n are as described herein (e.g. formula (I) to (XXV), including embodiments).

In some embodiments, R² and R⁶ substituents may optionally be joined to form a substituted or unsubstituted heterocycloalkyl or substituted or unsubstituted heteroaryl.

In some embodiments the compound having formula XVI is a compound having the formula:

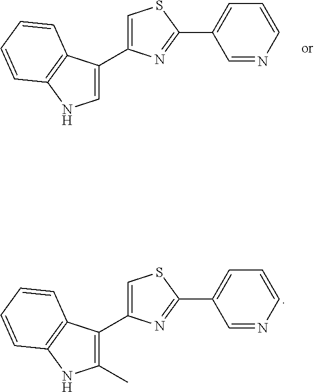 [SMILES: c1cncc(-c2nc(-c3c[nH]c4ccccc34)cs2)c1 ; Cc1[nH]c2ccccc2c1-c1csc(-c2cccnc2)n1]

In another aspect is a compound having the formula:

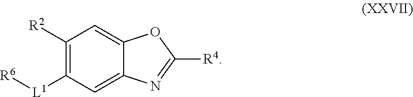

L¹, R^{1A}, R², R⁴, R⁶, R⁷, R⁸, R⁹, R¹⁰, X, m, v, and n are as described herein (e.g. formula (I) to (XXVI), including embodiments).

In some embodiments, R² and R⁶ substituents may optionally be joined to form a substituted or unsubstituted heterocycloalkyl or substituted or unsubstituted heteroaryl.

In some embodiments the compound having formula XXVII is a compound having the formula:

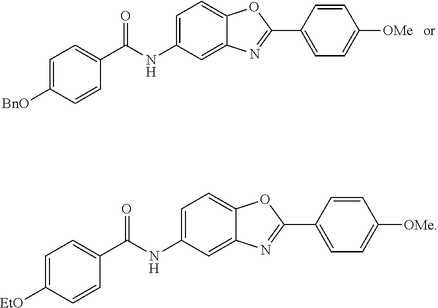 [SMILES: COc1ccc(-c2nc3cc(NC(=O)c4ccc(OCc5ccccc5)cc4)ccc3o2)cc1 ; "or" ; CCOc1ccc(C(=O)Nc2ccc3oc(-c4ccc(OC)cc4)nc3c2)cc1]

In another aspect is a compound having the formula:

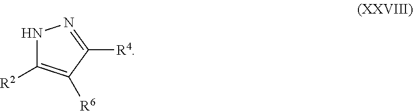

R², R⁴, R⁶, R⁷, R⁸, R⁹, R¹⁰, X, m, v, and n are as described herein (e.g. formula (I) to (XXVII), including embodiments).

In some embodiments, R² and R⁶ substituents may optionally be joined to form a substituted or unsubstituted heterocycloalkyl or substituted or unsubstituted heteroaryl. In some embodiments, R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl.

In some embodiments the compound having formula XXVIII is a compound having the formula:

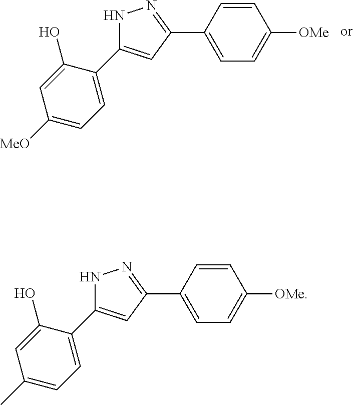 [SMILES: COc1ccc(-c2cc(-c3ccc(OC)cc3O)[nH]n2)cc1 ; "or" ; COc1ccc(-c2cc(-c3ccc(C)cc3O)[nH]n2)cc1]

In another aspect is a compound having the formula:

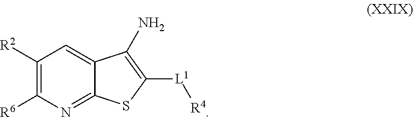

L¹, R^{1A}, R², R⁴, R⁶, R⁷, R⁸, R⁹, R¹⁰, X, m, v, and n are as described herein (e.g. formula (I) to (XXVIII), including embodiments).

In some embodiments, R² and R⁶ substituents may optionally be joined to form a substituted or unsubstituted heterocycloalkyl or substituted or unsubstituted heteroaryl.

In some embodiments the compound having formula XXIX is a compound having the formula:

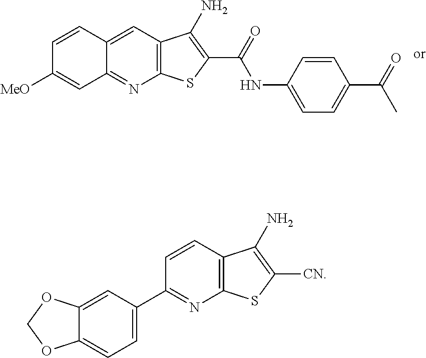 [SMILES: COc1ccc2cc3c(N)c(C(=O)Nc4ccc(C(C)=O)cc4)sc3nc2c1 ; "or" ; N#Cc1sc2nc(-c3ccc4c(c3)OCO4)ccc2c1N]

In another aspect is a compound having the formula:

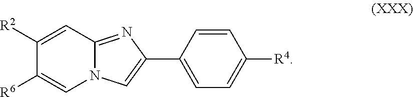

R², R⁴, R⁶, R⁷, R⁸, R⁹, R¹⁰, X, m, v, and n are as described herein (e.g. formula (I) to (XXIX), including embodiments).

In some embodiments, R² and R⁶ substituents may optionally be joined to form a substituted or unsubstituted heterocycloalkyl or substituted or unsubstituted heteroaryl.

In some embodiments the compound having formula XXX is a compound having the formula:

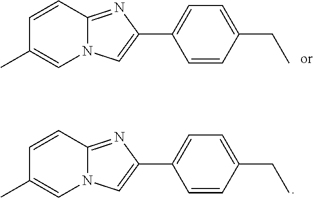

In another aspect is a compound having the formula:

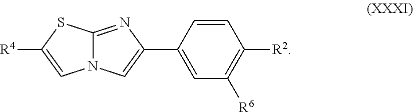

R², R⁴, R⁶, R⁷, R⁸, R⁹, R¹⁰, X, m, v, and n are as described herein (e.g. formula (I) to (XXX), including embodiments).

In some embodiments, R² and R⁶ substituents may optionally be joined to form a substituted or unsubstituted heterocycloalkyl or substituted or unsubstituted heteroaryl.

In some embodiments the compound having formula XXXI is a compound having formula:

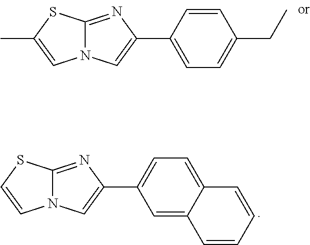 [SMILES: CCc1ccc(-c2cn3cc(C)sc3n2)cc1 ; c1ccc2cc(-c3cn4ccsc4n3)ccc2c1]

In another aspect is a compound having the formula:

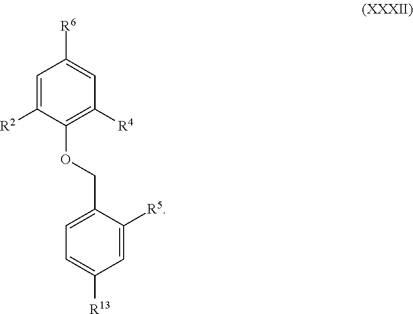

X, X^{a}, X^{b}, m, n, p, q, r, v, m1, v1, n1, R², R⁴, R⁵, R⁶, R⁷, R⁸, R⁹, R¹⁰, R^{7b}, R^{8b}, R^{9b}, R^{10b}, R¹³, R¹⁴, R¹⁵, R¹⁶, and R¹⁷ are as described herein (e.g. formula (I) to (XXXI), including embodiments).

In some embodiments, R² and R⁶ substituents may optionally be joined to form a substituted or unsubstituted heterocycloalkyl or substituted or unsubstituted heteroaryl. In some embodiments, R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl.

In some embodiments, the compound having formula XXXII is a compound having the formula:

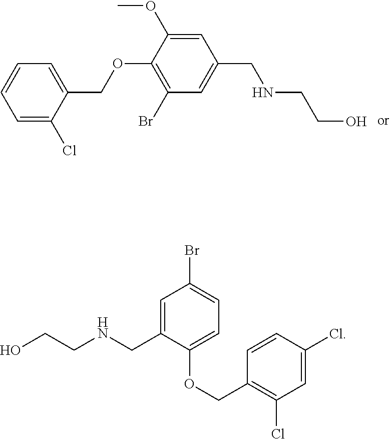 [SMILES: COc1cc(CNCCO)cc(Br)c1OCc1ccccc1Cl ; "or" ; OCCNCc1cc(Br)ccc1OCc1ccc(Cl)cc1Cl]

In another aspect is a compound having the formula:

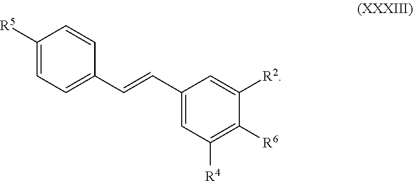

X, X^{b}, m, n, v, m1, v1, n1, R², R⁴, R⁵, R⁶, R⁷, R⁸, R⁹, R¹⁰R^{7b}, R^{8b}, R^{9b}, and R^{10b}, are as described herein (e.g. formula (I) to (XXXII), including embodiments).

In some embodiments, R² and R⁶ substituents may optionally be joined to form a substituted or unsubstituted heterocycloalkyl or substituted or unsubstituted heteroaryl. In some embodiments, R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl.

In some embodiments the compound having formula XXXIII is a compound having the formula:

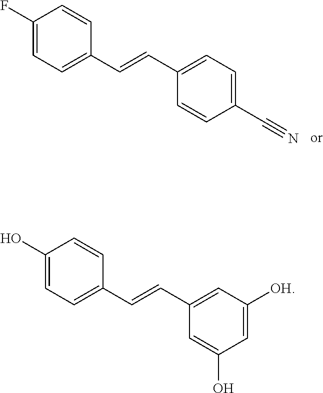 [SMILES: N#Cc1ccc(/C=C/c2ccc(F)cc2)cc1 ; "or" ; Oc1ccc(/C=C/c2cc(O)cc(O)c2)cc1]

In another aspect is a compound having the formula:

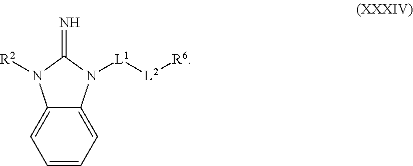

L¹, L², R^{1A}, R^{1B}, X, m, n, v, R², R⁶, R⁷, R⁸, R⁹, and R¹⁰ are as described herein (e.g. formula (I) to (XXXIII), including embodiments).

In some embodiments the compound having formula XXXIV is a compound having the formula:

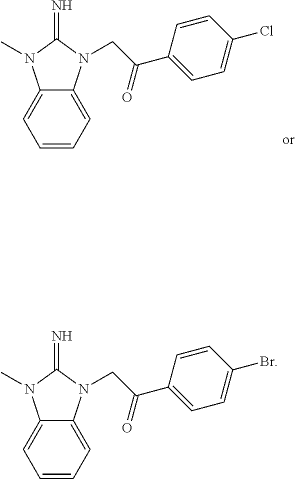 [SMILES: Cn1c(=N)n(CC(=O)c2ccc(Cl)cc2)c2ccccc21 ; Cn1c(=N)n(CC(=O)c2ccc(Br)cc2)c2ccccc21]

In another aspect is a compound having the formula:

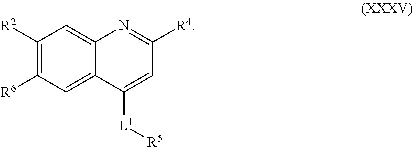

L¹, R^{1A}, X, X^{b}, m, n, v, m1, v1, n1, R², R⁴, R⁵, R⁶, R⁷, R⁸, R⁹, R¹⁰, R^{7b}, R^{8b}, R^{9b}, and R^{10b} are as described herein (e.g. formula (I) to (XXXIV), including embodiments).

In some embodiments, R² and R⁶ substituents may optionally be joined to form a substituted or unsubstituted heterocycloalkyl or substituted or unsubstituted heteroaryl.

In some embodiments the compound having formula XXXV is a compound having the formula:

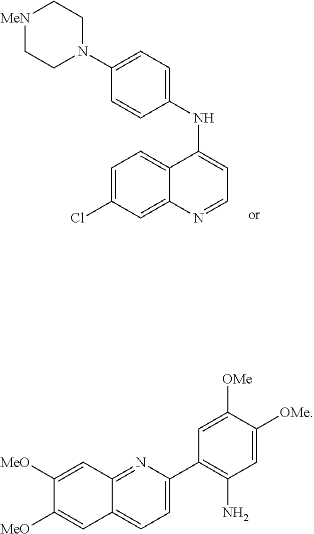 [SMILES: CN1CCN(c2ccc(Nc3ccnc4cc(Cl)ccc34)cc2)CC1 ; COc1cc(N)c(-c2ccc3cc(OC)c(OC)cc3n2)cc1OC]

In another aspect is a compound having the formula:

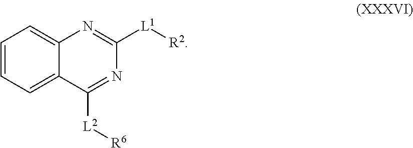

L¹, L², R^{1A}, R^{1B}, X, m, n, v, R², R⁶, R⁷, R⁸, R⁹, and R¹⁰ are as described herein (e.g. formula (I) to (XXXV), including embodiments).

In some embodiments, the compound having formula XXXVI is a compound having the formula:

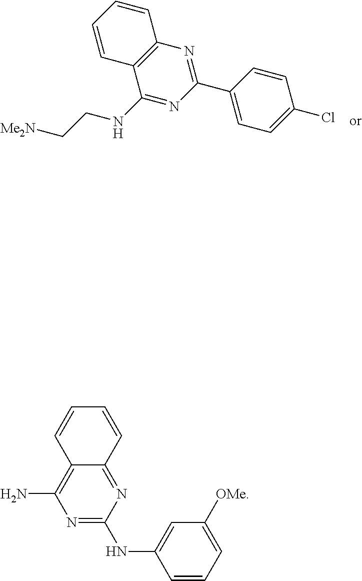 [SMILES: CN(C)CCNc1nc(-c2ccc(Cl)cc2)nc2ccccc12 ; COc1cccc(Nc2nc(N)c3ccccc3n2)c1]

In another aspect is a compound having the formula:

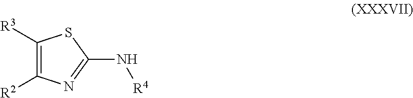

X, m, n, v, R², R³, R⁴, R⁷, R⁸, R⁹, and R¹⁰ are as described herein (e.g. formula (I) to (XXXVI), including embodiments).

In some embodiments, X, m, n, v, R², R³, R⁴, R⁷, R⁸, R⁹, and R¹⁰, are as described in the paragraphs below in a compound of formula (XXXVII). In some embodiments, these values are included in any other formula described herein. In some embodiments, R² is hydrogen. In some embodiments, R² is —OCH₃. In some embodiments, R² is substituted or unsubstituted alkyl. In some embodiments, R² is substituted alkyl. In some embodiments, R² is unsubstituted alkyl. In some embodiments, R² is substituted or unsubstituted heteroalkyl. In some embodiments, R² is substituted heteroalkyl. In some embodiments, R² is unsubstituted heteroalkyl.

In some embodiments, R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R² is substituted or unsubstituted alkyl. In some embodiments, R² is substituted or unsubstituted heteroalkyl. In some embodiments, R² is substituted or unsubstituted cycloalkyl. In some embodiments, R² is substituted or unsubstituted heterocycloalkyl. In some embodiments, R² is substituted or unsubstituted aryl. In some embodiments, R² is substituted or unsubstituted heteroaryl. In some embodiments, R² is unsubstituted alkyl. In some embodiments, R² is unsubstituted heteroalkyl. In some embodiments, R² is unsubstituted cycloalkyl. In some embodiments, R² is unsubstituted heterocycloalkyl. In some embodiments, R² is unsubstituted aryl. In some embodiments, R² is unsubstituted heteroaryl. In some embodiments, R² is substituted alkyl. In some embodiments, R² is substituted heteroalkyl. In some embodiments, R² is substituted cycloalkyl. In some embodiments, R² is substituted heterocycloalkyl. In some embodiments, R² is substituted aryl. In some embodiments, R² is substituted heteroaryl. In some embodiments, R² is R²³-substituted alkyl. In some embodiments, R² is R²³-substituted heteroalkyl. In some embodiments, R² is R²³-substituted cycloalkyl. In some embodiments, R² is R²³-substituted heterocycloalkyl. In some embodiments, R² is R²³-substituted aryl. In some embodiments, R² is R²³-substituted heteroaryl. In some embodiments, R² is substituted or unsubstituted phenyl. In some embodiments, R² is R²³-substituted or unsubstituted phenyl. In some embodiments, R² is substituted phenyl. In some embodiments, R² is unsubstituted phenyl. In some embodiments, R² is R²³-substituted phenyl. In some embodiments, R² is substituted or unsubstituted thienyl. In some embodiments, R² is R²³-substituted or unsubstituted thienyl. In some embodiments, R² is substituted thienyl. In some embodiments, R² is unsubstituted thienyl. In some embodiments, R² is R²³-substituted thienyl. In some embodiments, R² is substituted or unsubstituted 4,5,6,7-tetrahydrobenzo[b]thienyl. In some embodiments, R² is R²³-substituted or unsubstituted 4,5,6,7-tetrahydrobenzo[b]thienyl. In some embodiments, R² is substituted 4,5,6,7-tetrahydrobenzo[b]thienyl. In some embodiments, R² is unsubstituted 4,5,6,7-tetrahydrobenzo[b]thienyl. In some embodiments, R² is R²³-substituted 4,5,6,7-tetrahydrobenzo[b]thienyl. In some embodiments, R² is substituted or unsubstituted pyridyl. In some embodiments, R² is R²³-substituted or unsubstituted pyridyl. In some embodiments, R² is substituted pyridyl. In some embodiments, R² is unsubstituted pyridyl. In some embodiments, R² is R²³-substituted pyridyl. In some embodiments, R² is substituted or unsubstituted 1,3,4-thiadiazolyl. In some embodiments, R² is R²³-substituted or unsubstituted 1,3,4-thiadiazolyl. In some embodiments, R² is substituted 1,3,4-thiadiazolyl. In some embodiments, R² is unsubstituted 1,3,4-thiadiazolyl. In some embodiments, R² is R²³-substituted 1,3,4-thiadiazolyl. In some embodiments, R² is substituted or unsubstituted piperidinyl. In some embodiments, R² is R²³-substituted or unsubstituted piperidinyl. In some embodiments, R² is substituted piperidinyl. In some embodiments, R² is unsubstituted piperidinyl. In some embodiments, R² is R²³-substituted piperidinyl. In some embodiments, R² is substituted or unsubstituted piperazinyl. In some embodiments, R² is R²³-substituted or unsubstituted piperazinyl. In some embodiments, R² is substituted piperazinyl. In some embodiments, R² is unsubstituted piperazinyl. In some embodiments, R² is R²³-substituted piperazinyl. In some embodiments, R² is substituted or unsubstituted oxazolyl. In some embodiments, R² is R²³-substituted or unsubstituted oxazolyl. In some embodiments, R² is substituted oxazolyl. In some embodiments, R² is unsubstituted oxazolyl. In some embodiments, R² is R²³-substituted oxazolyl. In some embodiments, R² is substituted or unsubstituted thiazolyl. In some embodiments, R² is R²³-substituted or unsubstituted thiazolyl. In some embodiments, R² is substituted thiazolyl. In some embodiments, R² is unsubstituted thiazolyl. In some embodiments, R² is R²³-substituted thiazolyl. In some embodiments, R² is R²³-substituted or unsubstituted benzo[d]oxazolyl. In some embodiments, R² is R²³-substituted benzo[d]oxazolyl. In some embodiments, R² is unsubstituted benzo[d]oxazolyl. In some embodiments, R² is substituted or unsubstituted tetrahydrothienyl. In some embodiments, R² is R²³-substituted or unsubstituted tetrahydrothienyl. In some embodiments, R² is substituted tetrahydrothienyl. In some embodiments, R² is unsubstituted tetrahydrothienyl. In some embodiments, R² is R²³-substituted tetrahydrothienyl. In some embodiments, R² is substituted or unsubstituted 2,3-dihydro-1H-pyrazolyl. In some embodiments, R² is R²³-substituted or unsubstituted 2,3-dihydro-1H-pyrazolyl. In some embodiments, R² is substituted 2,3-dihydro-1H-pyrazolyl. In some embodiments, R² is unsubstituted 2,3-dihydro-1H-pyrazolyl. In some embodiments, R² is R²³-substituted 2,3-dihydro-1H-pyrazolyl. In some embodiments, R² is substituted or unsubstituted (C₁-C₄) alkyl. In some embodiments, R² is R²³-substituted or unsubstituted (C₁-C₄) alkyl. In some embodiments, R² is substituted (C₁-C₄) alkyl. In some embodiments, R² is unsubstituted (C₁-C₄) alkyl. In some embodiments, R² is R²³-substituted (C₁-C₄) alkyl. In some embodiments, R² is substituted or unsubstituted ethyl. In some embodiments, R² is R²³-substituted or unsubstituted ethyl. In some embodiments, R² is substituted ethyl. In some embodiments, R² is unsubstituted ethyl. In some embodiments, R² is R²³-substituted ethyl.

In some embodiments, R² is R²³-substituted or unsubstituted imidazolyl. In some embodiments, R² is substituted imidazolyl. In some embodiments, R² is unsubstituted imidazolyl. In some embodiments, R² is R²³-substituted or unsubstituted 4H-1,2,4-triazolyl. In some embodiments, R² is substituted 4H-1,2,4-triazolyl. In some embodiments, R² is unsubstituted 4H-1,2,4-triazolyl. In some embodiments, R² is R²³-substituted or unsubstituted triazolyl. In some embodiments, R² is substituted triazolyl. In some embodiments, R² is unsubstituted triazolyl. In some embodiments, R² is substituted or unsubstituted naphthyl. In some embodiments, R² is substituted or unsubstituted furanyl. In some embodiments, R² is substituted or unsubstituted quinolinyl. In some embodiments, R² is unsubstituted naphthyl. In some embodiments, R² is unsubstituted furanyl. In some embodiments, R² is unsubstituted quinolinyl. In some embodiments, R² is substituted naphthyl. In some embodiments, R² is substituted furanyl. In some embodiments, R² is substituted quinolinyl. In some embodiments, R² is R²³-substituted naphthyl. In some embodiments, R² is R²³-substituted furanyl. In some embodiments, R² is R²³-substituted quinolinyl. In some embodiments, R² is substituted or unsubstituted morpholinyl. In some embodiments, R² is R²³-substituted or unsubstituted morpholinyl. In some embodiments, R² is substituted morpholinyl. In some embodiments, R² is unsubstituted morpholinyl. In some embodiments, R² is R²³-substituted morpholinyl. In some embodiments, R² is substituted or unsubstituted piperazinyl. In some embodiments, R² is R²³-substituted or unsubstituted piperazinyl. In some embodiments, R² is substituted piperazinyl. In some embodiments, R² is unsubstituted piperazinyl. In some embodiments, R² is R²³-substituted piperazinyl. In some embodiments, R² is substituted or unsubstituted pyrazolyl (e.g. 1H-pyrazolyl). In some embodiments, R² is R²³-substituted or unsubstituted pyrazolyl (e.g. 1H-pyrazolyl). In some embodiments, R² is substituted pyrazolyl (e.g. 1H-pyrazolyl). In some embodiments, R² is unsubstituted pyrazolyl (e.g. 1H-pyrazolyl). In some embodiments, R² is R²³-substituted pyrazolyl (e.g. 1H-pyrazolyl). In some embodiments, R² is substituted or unsubstituted benzofuranyl. In some embodiments, R² is R²³-substituted or unsubstituted benzofuranyl. In some embodiments, R² is substituted benzofuranyl. In some embodiments, R² is unsubstituted benzofuranyl. In some embodiments, R² is R²³-substituted benzofuranyl. In some embodiments, R² is substituted or unsubstituted isoxazolyl. In some embodiments, R² is R²³-substituted or unsubstituted isoxazolyl. In some embodiments, R² is substituted isoxazolyl. In some embodiments, R² is unsubstituted isoxazolyl. In some embodiments, R² is R²³-substituted isoxazolyl. In some embodiments, R² is substituted or unsubstituted imidazo[2,1-b]thiazolyl. In some embodiments, R² is R²³-substituted or unsubstituted imidazo[2,1-b]thiazolyl. In some embodiments, R² is substituted imidazo[2,1-b]thiazolyl. In some embodiments, R² is unsubstituted imidazo[2,1-b]thiazolyl. In some embodiments, R² is R²³-substituted imidazo[2,1-b]thiazolyl. In some embodiments, R² is substituted or unsubstituted indolyl. In some embodiments, R² is R²³-substituted or unsubstituted indolyl. In some embodiments, R² is substituted indolyl. In some embodiments, R² is unsubstituted indolyl. In some embodiments, R² is R²³-substituted indolyl. In some embodiments, R² is substituted or unsubstituted 1H-imidazo[1,2-a]imidazolyl. In some embodiments, R² is R²³-substituted or unsubstituted 1H-imidazo[1,2-a]imidazolyl. In some embodiments, R² is substituted 1H-imidazo[1,2-a]imidazolyl. In some embodiments, R² is unsubstituted 1H-imidazo[1,2-a]imidazolyl. In some embodiments, R² is R²³-substituted 1H-imidazo[1,2-a]imidazolyl.

In some embodiments, R² is substituted with one R²³. In some embodiments, R² is substituted with two optionally different R²³. In some embodiments, R² is substituted with three optionally different R²³. In some embodiments, R² is substituted with four optionally different R²³. In some embodiments, two adjacent R²³ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²³ substituents are joined to form a substituted cycloalkyl. In some embodiments, two adjacent R²³ substituents are joined to form an unsubstituted cycloalkyl. In some embodiments, two adjacent R²³ substituents are joined to form a substituted heterocycloalkyl. In some embodiments, two adjacent R²³ substituents are joined to form an unsubstituted heterocycloalkyl. In some embodiments, two adjacent R²³ substituents are joined to form a substituted aryl. In some embodiments, two adjacent R²³ substituents are joined to form an unsubstituted aryl. In some embodiments, two adjacent R²³ substituents are joined to form a substituted heteroaryl. In some embodiments, two adjacent R²³ substituents are joined to form an unsubstituted heteroaryl.

In some embodiments, R²³ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, —CH₃, R²⁴-substituted or unsubstituted piperidinyl, R²⁴-substituted or unsubstituted piperazinyl, R²⁴-substituted or unsubstituted thiazolyl, R²⁴-substituted or unsubstituted oxazolyl, R²⁴-substituted or unsubstituted phenyl, R²⁴-substituted or unsubstituted thienyl, R²⁴-substituted or unsubstituted 4,5,6,7-tetrahydrobenzo[b]thienyl, R²⁴-substituted or unsubstituted pyridyl, or R²⁴-substituted or unsubstituted 1,3,4-thiadiazolyl. In some embodiments, R²³ is R²⁴-substituted or unsubstituted benzo[d]oxazolyl. In some embodiments, R²³ is R²⁴-substituted or unsubstituted imidazolyl. In some embodiments, R²³ is R²⁴-substituted imidazolyl. In some embodiments, R²³ is unsubstituted imidazolyl. In some embodiments, R²³ is R²⁴-substituted or unsubstituted 4H-1,2,4-triazolyl. In some embodiments, R²³ is R²⁴-substituted 4H-1,2,4-triazolyl. In some embodiments, R²³ is unsubstituted 4H-1,2,4-triazolyl. In some embodiments, R²³ is R²⁴-substituted or unsubstituted triazolyl. In some embodiments, R²³ is R²⁴-substituted triazolyl. In some embodiments, R²³ is unsubstituted triazolyl. In some embodiments, R²³ is independently R²⁴-substituted or unsubstituted alkyl, R²⁴-substituted or unsubstituted heteroalkyl, R²⁴-substituted or unsubstituted cycloalkyl, R²⁴ substituted or unsubstituted heterocycloalkyl, R²⁴-substituted or unsubstituted aryl, or R²⁴-substituted or unsubstituted heteroaryl. In some embodiments, R²³ is independently R²⁴-substituted alkyl. In some embodiments, R²³ is unsubstituted alkyl. In some embodiments, R²³ is R²⁴-substituted heteroalkyl. In some embodiments, R²³ is unsubstituted heteroalkyl. In some embodiments, R²³ is R²⁴-substituted cycloalkyl. In some embodiments, R²³ is unsubstituted cycloalkyl. In some embodiments, R²³ is R²⁴-substituted heterocycloalkyl. In some embodiments, R²³ is unsubstituted heterocycloalkyl. In some embodiments, R²³ is R²⁴-substituted aryl. In some embodiments, R²³ is unsubstituted aryl. In some embodiments, R²³ is R²⁴-substituted heteroaryl. In some embodiments, R²³ is unsubstituted heteroaryl. In some embodiments, R²³ is substituted with one R²⁴. In some embodiments, R²³ is substituted with two optionally different R²⁴. In some embodiments, R²³ is substituted with three optionally different R²⁴. In some embodiments, R²³ is substituted with four optionally different R²⁴. In some embodiments, R²³ is independently oxo. In some embodiments, R²³ is independently —Br. In some embodiments, R²³ is independently —F. In some embodiments, R²³ is independently —Cl. In some embodiments, R²³ is independently —I. In some embodiments, R²³ is independently —CH₃. In some embodiments, R²³ is independently —OCH₃. In some embodiments, R²³ is independently (C₁-C₄) alkyl. In some embodiments, R²³ is independently (C₁-C₈) alkyl. In some embodiments, R²³ is independently (C₇-C₁₀) alkyl. In some embodiments, R²³ is independently (C₆-C₁₂) alkyl. In some embodiments, R²³ is independently phenyl. In some embodiments, R²³ is independently —OH. In some embodiments, R²³ is independently —CF₃.

In some embodiments, two adjacent R²⁴ substituents may optionally be joined to form a substituted or unsubstituted cycloalkyl, substituted or unsubstituted heterocycloalkyl, substituted or unsubstituted aryl, or substituted or unsubstituted heteroaryl. In some embodiments, two adjacent R²⁴ substituents are joined to form a substituted cycloalkyl. In some embodiments, two adjacent R²⁴ substituents are joined to form an unsubstituted cycloalkyl. In some embodiments, two adjacent R²⁴ substituents are joined to form a substituted heterocycloalkyl. In some embodiments, two adjacent R²⁴ substituents are joined to form an unsubstituted heterocycloalkyl. In some embodiments, two adjacent R²⁴ substituents are joined to form a substituted aryl. In some embodiments, two adjacent R²⁴ substituents are joined to form an unsubstituted aryl. In some embodiments, two adjacent R²⁴ substituents are joined to form a substituted heteroaryl. In some embodiments, two adjacent R²⁴ substituents are joined to form an unsubstituted heteroaryl.

In some embodiments, R²⁴ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —Cl, —F, —I, —Br, —OCH₂CH₃, —OCH₃, —OCF₃, —CH₂CH₃, or —CH₃. In some embodiments, R²⁴ is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, or R²⁵-substituted or unsubstituted heteroaryl. In some embodiments, R²⁴ is CH₃. In some embodiments, R²⁴ is independently R²⁵-substituted or unsubstituted piperidinyl, R²⁵-substituted or unsubstituted piperazinyl, R²⁵-substituted or unsubstituted thiazolyl, R²⁵-substituted or unsubstituted oxazolyl, R²⁵-substituted or unsubstituted phenyl, R²⁵-substituted or unsubstituted thienyl, R²⁵-substituted or unsubstituted 4,5,6,7-tetrahydrobenzo[b]thienyl, R²⁵-substituted or unsubstituted pyridyl, or R²⁵-substituted or unsubstituted 1,3,4-thiadiazolyl. In some embodiments, R²⁴ is R²⁵-substituted or unsubstituted benzo[d]oxazolyl. In some embodiments, R²⁴ is R²⁵-substituted or unsubstituted imidazolyl. In some embodiments, R²⁴ is R²⁵-substituted imidazolyl. In some embodiments, R²⁴ is unsubstituted imidazolyl. In some embodiments, R²⁴ is R²⁵-substituted or unsubstituted 4H-1,2,4-triazolyl. In some embodiments, R²⁴ is R²⁵-substituted 4H-1,2,4-triazolyl. In some embodiments, R²⁴ is unsubstituted 4H-1,2,4-triazolyl. In some embodiments, R²⁴ is R²⁵-substituted or unsubstituted triazolyl. In some embodiments, R²⁴ is R²⁵-substituted triazolyl. In some embodiments, R²⁴ is unsubstituted triazolyl. In some embodiments, R²⁴ is independently R²⁵-substituted or unsubstituted alkyl, R²⁵-substituted or unsubstituted heteroalkyl, R²⁵-substituted or unsubstituted cycloalkyl, R²⁵ substituted or unsubstituted heterocycloalkyl, R²⁵-substituted or unsubstituted aryl, or R²⁵-substituted or unsubstituted heteroaryl. In some embodiments, R²⁴ is independently R²⁵-substituted alkyl. In some embodiments, R²⁴ is unsubstituted alkyl. In some embodiments, R²⁴ is R²⁵-substituted heteroalkyl. In some embodiments, R²⁴ is unsubstituted heteroalkyl. In some embodiments, R²⁴ is R²⁵-substituted cycloalkyl. In some embodiments, R²⁴ is unsubstituted cycloalkyl. In some embodiments, R²⁴ is R²⁵-substituted heterocycloalkyl. In some embodiments, R²⁴ is unsubstituted heterocycloalkyl. In some embodiments, R²⁴ is R²⁵-substituted aryl. In some embodiments, R²⁴ is unsubstituted aryl. In some embodiments, R²⁴ is R²⁵-substituted heteroaryl. In some embodiments, R²⁴ is unsubstituted heteroaryl. In some embodiments, R²⁴ is substituted with one R²⁵. In some embodiments, R²⁴ is substituted with two optionally different R²⁵. In some embodiments, R²⁴ is substituted with three optionally different R²⁵. In some embodiments, R²⁴ is substituted with four optionally different R²⁵. In some embodiments, R²⁴ is independently oxo. In some embodiments, R²⁴ is independently —Br. In some embodiments, R²⁴ is independently —F. In some embodiments, R²⁴ is independently —Cl. In some embodiments, R²⁴ is independently —I. In some embodiments, R²⁴ is independently —CH₃. In some embodiments, R²⁴ is independently —OCH₃. In some embodiments, R²⁴ is independently (C₁-C₄) alkyl. In some embodiments, R²⁴ is independently (C₁-C₈) alkyl. In some embodiments, R²⁴ is independently (C₇-C₁₀) alkyl. In some embodiments, R²⁴ is independently (C₆-C₁₂) alkyl. In some embodiments, R²⁴ is independently phenyl. In some embodiments, R²⁴ is independently —OH. In some embodiments, R²⁴ is independently —CF₃. In some embodiments, R²⁴ is R²⁵-substituted or unsubstituted benzo[d]oxazolyl. In some embodiments, R²⁴ is R²⁵-substituted benzo[d]oxazolyl. In some embodiments, R²⁴ is unsubstituted benzo[d]oxazolyl. In some embodiments, R²⁴ is R²⁵-substituted or unsubstituted imidazolyl. In some embodiments, R²⁴ is R²⁵-substituted imidazolyl. In some embodiments, R²⁴ is unsubstituted imidazolyl. In some embodiments, R²⁴ is R²⁵-substituted or unsubstituted 4H-1,2,4-triazolyl. In some embodiments, R²⁴ is R²⁵-substituted 4H-1,2,4-triazolyl. In some embodiments, R²⁴ is unsubstituted 4H-1,2,4-triazolyl. In some embodiments, R²⁴ is R²⁵-substituted or unsubstituted triazolyl. In some embodiments, R²⁴ is R²⁵-substituted triazolyl. In some embodiments, R²⁴ is unsubstituted triazolyl. In some embodiments, R²⁴ is R²⁵-substituted or unsubstituted (C₁-C₄) alkyl. In some embodiments, R²⁴ is 2-propyl. In some embodiments, R²⁴ is methyl. In some embodiments, R²⁴ is ethyl. In some embodiments, R²⁴ is propyl. In some embodiments, R²⁴ is butyl. In some embodiments, R²⁴ is cyclopropyl. In some embodiments, R²⁴ is cyclobutyl. Where each R⁷, R⁸, R⁹, and R¹⁰, X, n, v, and m is different, they may be referred to, for example, as R^{7c}, R^{7d}, R^{7e}, R^{7f}, R^{7g}, R^{7h}, R⁷ⁱ, R^{8c}, R^{8d}, R^{8e}, R^{8f}, R^{8g}, R^{8h}, R⁸ⁱ, R^{9c}, R^{9d}, R^{9e}, R^{9f}, R^{9g}, R^{9h}, R⁹ⁱ, R^{10c}, R^{10d}, R^{10e}, R^{10f}, R^{10g}, R^{10h}, R¹⁰ⁱ, X^{c}, X^{d}, X^{e}, X^{f}, X^{g}, X^{h}, Xⁱ, n^{c}, n^{d}, n^{e}, n^{f}, n^{g}, n^{h}, nⁱ, v^{c}, v^{d}, v^{e}, v^{f}, v^{g}, V^{h}, vⁱ, m^{c}, m^{d}, m^{e}, m^{f}, m^{g}, m^{h}, mⁱ, and so on, wherein each R^{7c}, R^{7d}, R^{7e}, R^{7f}, R^{7g}, R^{7h}, R⁷ⁱ is defined the same as R⁷, each R^{8c}, R^{8d}, R^{8e}, R^{8f}, R^{8g}, R^{8h}, R⁸ⁱ is defined the same as R⁸, each R^{9c}, R^{9d}, R^{9e}, R^{9f}, R^{9g}, R^{9h}, R⁹ⁱ is defined the same as R⁹, each R^{10c}, R^{10d}, R^{10e}, R^{10f}, R^{10g}, R^{10h}, R¹⁰ⁱ is defined the same as R¹⁰, each X^{c}, X^{d}, X^{e}, X^{f}, X^{g}, X^{h}, Xⁱ is defined the same as X, each n^{c}, n^{d}, n^{e}, n^{f}, n^{g}, n^{h}, nⁱ is defined the same as n, each v^{c}, v^{d}, v^{f}, v^{g}, v^{h}, vⁱ is defined the same as v, each m^{c}, m^{d}, m^{e}, m^{f}, m^{g}, m^{h}, mⁱ is defined the same as m. In some embodiments, R² is defined by R^{7c}, R^{8c}, R^{9c}, R^{10c}, X^{c}, n^{c}, v^{c}, and m^{c}. In some embodiments, R³ is defined by R^{7d}, R^{8d}, R^{9d}, R^{10d}, X^{d}, n^{d}, v^{d}, and m^{d}. In some embodiments, R⁴ is defined by R^{7e}, R^{8e}, R^{9e}, R^{10e}, X^{e}, n^{e}, v^{e}, and m^{e}. In some embodiments, R⁶ is defined by R^{7f}, R^{8f}, R^{9f}, R^{10f}, X^{f}, n^{f}, v^{f}, and m^{f}. In some embodiments, R¹ is defined by R^{7g}, R^{8g}, R^{9g}, R^{10g}, X^{g}, n^{g}, v^{g}, and m^{g}. In some embodiments, R^{1A} is defined by R^{7h}, R^{8h}, R^{9h}, R^{10h}, X^{h}, n^{h}, and m^{h}. In some embodiments, R^{1B} is defined by R⁷ⁱ, R⁸ⁱ, R⁹ⁱ, R¹⁰ⁱ, Xⁱ, nⁱ, vⁱ, and mⁱ. Where c, d, e, f, g, h, and i denote substituents of R², R³, R⁴, R⁶, R¹¹, R^{1A}, and R^{1B} respectively.

In some embodiments the compound having formula XXXVII is a compound having the formula:

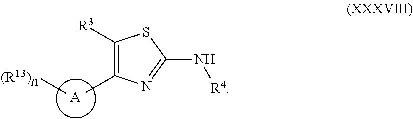

Ring A, R³, R⁴, R⁷, R⁸, R⁹, R¹⁰, R¹³, R¹⁴, R¹⁵, R¹⁶, R¹⁷, X, X^{a}, m, p, v, n, r, q, and t1 are as described herein (e.g. formula (I) to (XXXVII), including embodiments).

In some embodiments, two adjacent R¹³ substituents may optionally be joined to form a substituted or unsubstitued cycloalkyl, substituted or unsubstituted heterocycloalkyl, substituted or unsubstituted aryl, or substituted or unsubstituted heteroaryl

In some embodiments, the compound having formula XXXVII is a compound having the formula:

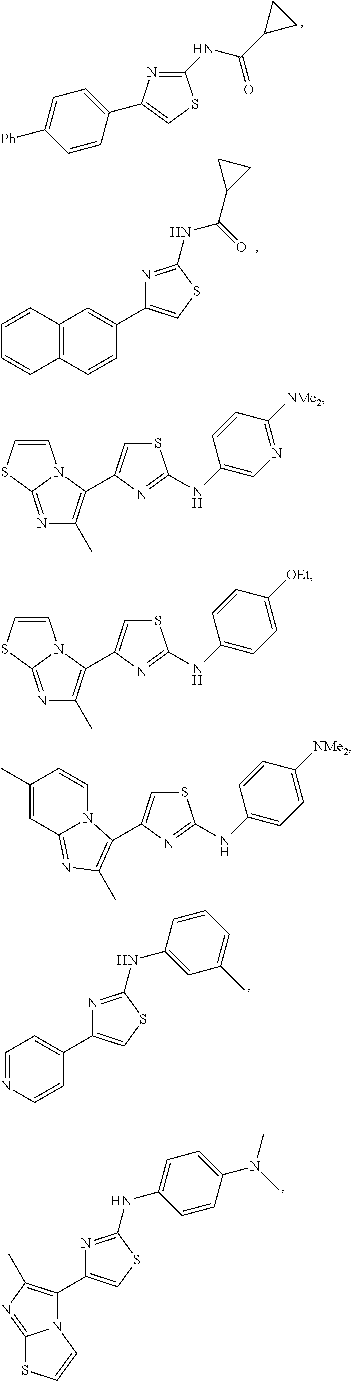

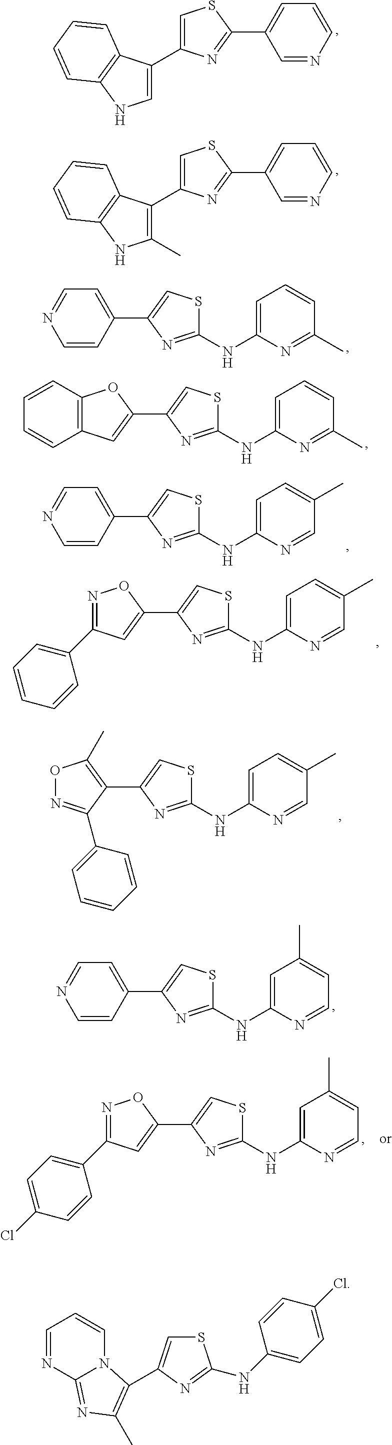

In another aspect is a compound having the formula:

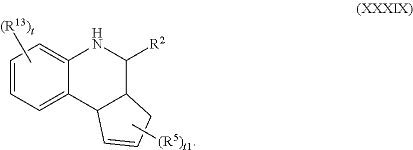

X, X^{a}, X^{b}, m, n, p, q, r, v, m1, v1, n1, t, t1, R², R⁵, R⁶, R⁷, R⁸, R⁹, R¹⁰, R^{7b}, R^{8b}, R^{9b}, R^{10b}, R¹³, R¹⁴, R¹⁵, R¹⁶, and R¹⁷ are as described herein (e.g. formula (I) to (XXXVIII), including embodiments).

In some embodiments, two adjacent R⁵ substituents may optionally be joined to form a substituted or unsubstitued cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstitued cycloalkyl, substituted or unsubstituted heterocycloalkyl, substituted or unsubstituted aryl, or substituted or unsubstituted heteroaryl.

In some embodiments, the compound having formula XXXIX is a compound having the formula:

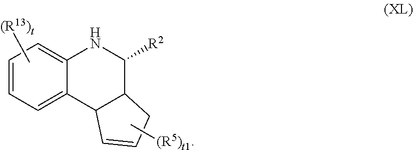

R², R⁵, R⁷, R⁸, R⁹, R¹⁰, R^{7b}, R^{8b}, R^{9b}, R^{10b}, R¹³, R¹⁴, R¹⁵, R¹⁶, R¹⁷, X, X^{a}, X^{b}, n, n1, v, v1, m, m1, r, p, q, t, and t1 are as described herein (e.g. formula (I) to (XXXIX), including embodiments).

In some embodiments, two adjacent R⁵ substituents may optionally be joined to form a substituted or unsubstitued cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstitued cycloalkyl, substituted or unsubstituted heterocycloalkyl, substituted or unsubstituted aryl, or substituted or unsubstituted heteroaryl.

In some embodiments, the compound having formula XXXIX is a compound having the formula:

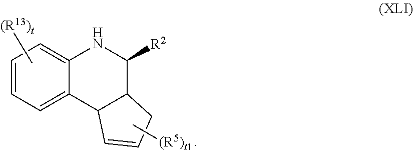

R², R⁵, R⁷, R⁸, R⁹, R¹⁰, R^{7b}, R^{8b}, R^{9b}, R^{10b}, R¹³, R¹⁴, R¹⁵, R¹⁶, R¹⁷, X, X^{a}, X^{b}, n, n1, v, v1, m, m1, r, p, q, t, and t1 are as described herein (e.g. formula (I) to (XL), including embodiments).

In some embodiments, two adjacent R⁵ substituents may optionally be joined to form a substituted or unsubstitued cycloalkyl, substituted or unsubstituted heterocycloalkyl, substituted or unsubstituted aryl, or substituted or unsubstituted heteroaryl. In some embodiments, two adjacent R¹³ substituents may optionally be joined to form a substituted or unsubstitued cycloalkyl, substituted or unsubstituted heterocycloalkyl, substituted or unsubstituted aryl, or substituted or unsubstituted heteroaryl.

In some embodiments, the compound having formula XXXIX is a compound having the formula:

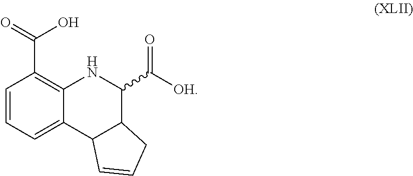 [SMILES: O=C(O)c1cccc2c1NC(C(=O)O)C1CC=CC21]

In another aspect is a compound of Table 2, 4, 5, 6, 8, 9, 18, 19, 20, 24, 25, 26, 27, 28, FIG. 20, 31, Chart 1, 2, 3, 4, or 5. In the compounds of formula (I) through (XLII) and any compound selected from the compounds of formula (I) through (XLII), including any embodiments, wherein the compound(s) are described herein using any of the variables R⁷, R⁸, R⁹, R¹⁰, X, n, v, m, R², R³, R⁴, R⁶, R¹, R^{1A}, or R^{1B} and where each R⁷, R⁸, R⁹, and R¹⁰, X, n, v, and m is different, they may be referred to, for example, as R^{7c}, R^{7d}, R^{7e}, R^{7f}, R^{7g}, R^{7h}, R⁷ⁱ, R^{8c}, R^{8d}, R^{8e}, R^{8f}, R^{8g}, R^{8h}, R⁸ⁱ, R^{9c}, R^{9d}, R^{9e}, R^{9f}, R^{9g}, R^{9h}, R⁹ⁱ, R^{10c}, R^{10d}, R^{10e}, R^{10f}, R^{10g}, R^{10h}, R¹⁰ⁱ, X^{c}, X^{d}, X^{e}, X^{f}, X^{g}, X^{h}, Xⁱ, n^{c}, n^{d}, n^{e}, n^{f}, n^{g}, n^{h}, nⁱ, v^{c}, v^{d}, v^{e}, v^{f}, v^{g}, v^{h}, vⁱ, m^{c}, m^{d}, m^{e}, m^{f}, m^{g}, m^{h}, mⁱ, and so on, wherein each R^{7c}, R^{7d}, R^{7e}, R^{7f}, R^{7g}, R^{7h}, R⁷ⁱ is defined the same as R⁷, each R^{8c}, R^{8d}, R^{8e}, R^{8f}, R^{8g}, R^{8h}, R⁸ⁱ is defined the same as R⁸, each R^{9c}, R^{9d}, R^{9e}, R^{9f}, R^{9g}, R^{9h}, R⁹ⁱ is defined the same as R⁹, each R^{10c}, R^{10d}, R^{10e}, R^{10f}, R^{10g}, R^{10h}, R¹⁰ⁱ is defined the same as R¹⁰, each X^{c}, X^{d}, X^{e}, X^{f}, X^{g}, X^{h}, Xⁱ is defined the same as X, each n^{c}, n^{d}, n^{e}, n^{f}, n^{g}, n^{h}, nⁱ is defined the same as n, each v^{c}, v^{d}, v^{f}, v^{g}, v^{h}, vⁱ is defined the same as v, each m^{c}, m^{d}, m^{e}, m^{f}, m^{g}, m^{h}, mⁱ is defined the same as m. In some embodiments, R² is defined by R^{7c}, R^{8c}, R^{9c}, R^{10c}, X^{c}, n^{c}, v^{c}, and m^{c}. In some embodiments, R³ is defined by R^{7d}, R^{8d}, R^{9d}, R^{10d}, X^{d}, n^{d}, v^{d}, and m^{d}. In some embodiments, R⁴ is defined by R^{7e}, R^{8e}, R^{9e}, R^{10e}, X^{e}, n^{e}, v^{e}, and m^{e}. In some embodiments, R⁶ is defined by R^{7f}, R^{8f}, R^{9f}, R^{10f}, X^{f}, n^{f}, v^{f}, and m^{f}. In some embodiments, R¹ is defined by R^{7g}, R^{8g}, R^{9g}, R^{10g}, X^{g}, n^{g}, v^{g}, and m^{g}. In some embodiments, R^{1A} is defined by R^{7h}, R^{8h}, R^{9h}, R^{10h}, X^{h}, V^{h}, and m^{h}. In some embodiments, R^{1B} is defined by R⁷ⁱ, R⁸ⁱ, R⁹ⁱ, R¹⁰ⁱ, Xⁱ, nⁱ, vⁱ, and mⁱ. Where c, d, e, f, g, h, and i denote substituents of R², R³, R⁴, R⁶, R¹¹, R^{1A}, and R^{1B} respectively. In the compounds of formula (I) through (XLII) and any compound selected from the compounds of formula (I) through (XLII), including any embodiments, wherein the compound(s) are described herein using any of the variables R¹⁴, R¹⁵, R¹⁶, R¹⁷, X^{a}, r, p, q, or R¹³, and where each R¹⁴, R¹⁵, R¹⁶, and R¹⁷, X^{a}, r, p, and q is different, they may be referred to, for example, as R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R¹³ is defined by R^{14c}, R^{15c}, R^{16c}, R^{17c}, X^{ac}, r^{c}, p^{c}, and q^{c}. In some embodiments, R¹³ is defined by R^{14d}, R^{15d}, R^{16d}, R^{17d}, X^{ad}, r^{d}, p^{d}, and q^{d}. In some embodiments, R¹³ is defined by R^{14e}, R^{15e}, R^{16e}, and R^{17e}, X^{ae}, r^{e}, p^{e}, and q^{e}. In some embodiments, R¹³ is defined by R^{14f}, R^{15f}, R^{16f}, and R^{17f}, X^{af}, r^{f}, p^{f}, and q^{f}. In some embodiments, R¹³ is defined by R^{14g}, R^{15g}, R^{16g}, and R^{17g}, X^{ag}, r^{g}, p^{g}, and q^{g}. Where c, d, e, f, and g denote substituents of different R¹³ respectively. In the compounds of formula (I) through (XLII) and any compound selected from the compounds of formula (I) through (XLII), including any embodiments, wherein the compound(s) are described herein using any of the variables R¹⁴, R¹⁵, R¹⁶, R¹⁷, X^{a}, r, p, q, R^{13a}, R^{13b}, or R^{13c}, and where each R¹⁴, R¹⁵, R¹⁶, and R¹⁷, X^{a}, r, p, and q is different, they may be referred to, for example, as R^{14c}, R^{14d}, R^{14e}, R^{14f}, R^{14g}, R^{15c}, R^{15d}, R^{15e}, R^{15f}, R^{15g}, R^{16c}, R^{16d}, R^{16e}, R^{16f}, R^{16g}, R^{17c}, R^{17d}, R^{17e}, R^{17f}, R^{17g}, X^{ac}, X^{ad}, X^{ae}, X^{af}, X^{ag}, r^{c}, r^{d}, r^{e}, r^{f}, r^{g}, p^{c}, p^{d}, p^{e}, p^{f}, p^{g}, q^{c}, q^{d}, q^{e}, q^{f}, q^{g}, and so on, wherein each R^{14c}, R^{14d}, R^{14e}, R^{14f}, R^{14g} is defined the same as R¹⁴, each R^{15c}, R^{15d}, R^{15e}, R^{15f}, R^{15g} is defined the same as R¹⁵, each R^{16c}, R^{16d}, R^{16e}, R^{16f}, R^{16g} is defined the same as R¹⁶, each R^{17c}, R^{17d}, R^{17e}, R^{17f}, R^{17g} is defined the same as R¹⁷, each X^{ac}, X^{ad}, X^{ae}, X^{af}, X^{ag} is defined the same as X^{a}, each r^{c}, r^{d}, r^{e}, r^{f}, r^{g} is defined the same as r, each p^{c}, p^{d}, p^{e}, p^{f}, p^{g} is defined the same as p, each q^{c}, q^{d}, q^{e}, q^{f}, q^{g} is defined the same as q. In some embodiments, R¹³ is defined by R^{14c}, R^{15c}, R^{16c}, R^{17c}, X^{ac}, r^{c}, p^{c}, and q^{c}. In some embodiments, R¹³ is defined by R^{14d}, R^{15d}, R^{16d}, R^{17d}, X^{ad}, r^{d}, p^{d}, and q^{d}. In some embodiments, R¹³ is defined by R^{14e}, R^{15e}, R^{16e}, and R^{17e}, X^{ae}, r^{e}, p^{e}, and q^{e}. Where c, d, and e denote substituents of R^{13a}, R^{13b}, and R^{13c} respectively. In the compounds of formula (I) through (XLII) and any compound selected from the compounds of formula (I) through (XLII), including any embodiments, wherein the compound(s) are described herein using any of the variables R^{7b}, R^{8b}, R^{9b}, and R^{10b}, X^{b}, n1, v1, and m1, or R⁵, and where each R^{7b}, R^{8b}, R^{9b}, and R^{10b}, X^{b}, n1, v1, and m1 is different, they may be refereed to, for example, as R^{7bc}, R^{7bd}, R^{7be}, R^{bf}, R^{7bg}, R^{8bc}, R^{8bd}, R^{8be}, R^{8bf}, R^{8bg}, R^{9bc}, R^{9bd}, R^{9be}, R^{9bf}, R_{9bg}, R^{10bc}, R^{10bc}, R^{10be}, R^{10bf}, R^{10bg}, X^{bc}, X^{bd}, X^{be}, X^{bf}, X^{bg}, n1^{c}, n1^{d}, n1^{e}, n1^{f}, n1^{g}, v1^{c}, v1^{d}, v1^{e}, v1^{f}, v1^{g}, m1^{c}, m1^{d}, m1^{e}, m1^{f}, m1^{g}, and so on, wherein each R^{7bc}, R^{7bd}, R^{7be}, R^{7bf}, R^{7bg} is defined the same as R^{7b}, each R^{8bc}, R^{8bd}, R^{8be}, R^{8bf}, R^{8bg} is defined the same as R^{8b}, each R^{9bc}, R^{9bd}, R^{9be}, R^{9bf}, R^{9bg} is defined the same as R^{9b}, each R^{10bc}, R^{10bd}, R^{10be}, R^{10bf}, R^{10bg} is defined the same as R^{10b}, each X^{bc}, X^{bd}, X^{be}, X^{bf}, X^{bg} is defined the same as X^{b}, each n1^{c}, n1^{d}, n1^{e}, n1^{f}, n1^{g} is defined the same as n1, each v1^{c}, v1^{d}, v1^{e}, v1^{f}, v1^{g} is defined the same as v1, each m1^{c}, m1^{d}, m1^{e}, m1^{f}, m1^{g} is defined the same as m1. In some embodiments, R⁵ is defined by R^{7bc}, R^{8bc}, R^{9bc}, R^{10bc}, X^{bc}, n1^{c}, v1^{c}, and m1^{c}. In some embodiments, R⁵ is defined by R^{7bd}, R^{8bd}, R^{9bd}, R^{10bd}, X^{bd}, n1^{d}, v1^{d}, m1^{d}. In some embodiments, R⁵ is defined by R^{7be}, R^{8be}, R^{9be}, R^{10be}, X^{be}, n1^{e}, v1^{e}, and m1^{e}. In some embodiments, R⁵ is defined by R^{7bf}, R^{8bf}, R^{9bf}, R^{10bf}, X^{bf}, n1^{f}, v1^{f} and m1^{f}. In some embodiments, R⁵ is defined by R^{7bg}, R^{8bg}, R^{9bg}, R^{10bg}, X^{bg}, n1^{g}, v1^{g}, and m1^{g}. Where c, d, e, f, and g denote substituents of different R⁵ respectively.

In some embodiments of the compounds provided herein, R¹ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²⁰-substituted or unsubstituted alkyl, R²⁰-substituted or unsubstituted heteroalkyl, R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl. In some embodiments, two adjacent R¹ substituents may optionally be joined to form a substituted (e.g. R²⁰-substituted) or unsubstituted cycloalkyl, substituted (e.g. R²⁰-substituted) or unsubstituted heterocycloalkyl, substituted (e.g. R²⁰-substituted) or unsubstituted aryl, or substituted (e.g. R²⁰-substituted) or unsubstituted heteroaryl.

R²⁰ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl. Two adjacent R²⁰ substituents may optionally be joined to form a substituted (e.g. R²¹-substituted) or unsubstituted cycloalkyl, substituted (e.g. R²¹-substituted) or unsubstituted heterocycloalkyl, substituted (e.g. R²¹-substituted) or unsubstituted aryl, or substituted (e.g. R²¹-substituted) or unsubstituted heteroaryl.

R²¹ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²²-substituted or unsubstituted alkyl, R²²-substituted or unsubstituted heteroalkyl, R²²-substituted or unsubstituted cycloalkyl, R²²-substituted or unsubstituted heterocycloalkyl, R²²-substituted or unsubstituted aryl, or R²²-substituted or unsubstituted heteroaryl. Two adjacent R²¹ substituents may optionally be joined to form a substituted (e.g. R²²-substituted) or unsubstituted cycloalkyl, substituted (e.g. R²²-substituted) or unsubstituted heterocycloalkyl, substituted (e.g. R²²-substituted) or unsubstituted aryl, or substituted (e.g. R²²-substituted) or unsubstituted heteroaryl.

In some embodiments of the compounds provided herein, R² is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²³-substituted or unsubstituted alkyl, R²³-substituted or unsubstituted heteroalkyl, R²³-substituted or unsubstituted cycloalkyl, R²³-substituted or unsubstituted heterocycloalkyl, R²³-substituted or unsubstituted aryl, or R²³-substituted or unsubstituted heteroaryl. In some embodiments, two adjacent R² substituents may optionally be joined to form a substituted (e.g. R²³-substituted) or unsubstituted cycloalkyl, substituted (e.g. R²³-substituted) or unsubstituted heterocycloalkyl, substituted (e.g. R²³-substituted) or unsubstituted aryl, or substituted (e.g. R²³-substituted) or unsubstituted heteroaryl.

R²³ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²⁴-substituted or unsubstituted alkyl, R²⁴-substituted or unsubstituted heteroalkyl, R²⁴-substituted or unsubstituted cycloalkyl, R²⁴-substituted or unsubstituted heterocycloalkyl, R²⁴-substituted or unsubstituted aryl, or R²⁴-substituted or unsubstituted heteroaryl. Two adjacent R²³ substituents may optionally be joined to form a substituted (e.g. R²⁴-substituted) or unsubstituted cycloalkyl, substituted (e.g. R²⁴-substituted) or unsubstituted heterocycloalkyl, substituted (e.g. R²⁴-substituted) or unsubstituted aryl, or substituted (e.g. R²⁴-substituted) or unsubstituted heteroaryl.

R²⁴ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, or R²⁵-substituted or unsubstituted heteroaryl. Two adjacent R²⁴ substituents may optionally be joined to form a substituted (e.g. R²⁵-substituted) or unsubstituted cycloalkyl, substituted (e.g. R²⁵-substituted) or unsubstituted heterocycloalkyl, substituted (e.g. R²⁵-substituted) or unsubstituted aryl, or substituted (e.g. R²⁵-substituted) or unsubstituted heteroaryl.

In some embodiments of the compounds provided herein, R³ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²⁶-substituted or unsubstituted alkyl, R²⁶-substituted or unsubstituted heteroalkyl, R²⁶-substituted or unsubstituted cycloalkyl, R²⁶-substituted or unsubstituted heterocycloalkyl, R²⁶-substituted or unsubstituted aryl, or R²⁶-substituted or unsubstituted heteroaryl. In some embodiments, two adjacent R³ substituents may optionally be joined to form a substituted (e.g. R²⁶-substituted) or unsubstituted cycloalkyl, substituted (e.g. R²⁶-substituted) or unsubstituted heterocycloalkyl, substituted (e.g. R²⁶-substituted) or unsubstituted aryl, or substituted (e.g. R²⁶-substituted) or unsubstituted heteroaryl.

R²⁶ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²⁷-substituted or unsubstituted alkyl, R²⁷-substituted or unsubstituted heteroalkyl, R²⁷-substituted or unsubstituted cycloalkyl, R²⁷ substituted or unsubstituted heterocycloalkyl, R²⁷-substituted or unsubstituted aryl, or R²⁷-substituted or unsubstituted heteroaryl. Two adjacent R²⁶ substituents may optionally be joined to form a substituted (e.g. R²⁷-substituted) or unsubstituted cycloalkyl, substituted (e.g. R²⁷-substituted) or unsubstituted heterocycloalkyl, substituted (e.g. R²⁷-substituted) or unsubstituted aryl, or substituted (e.g. R²⁷-substituted) or unsubstituted heteroaryl.

R²⁷ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R²⁸-substituted or unsubstituted alkyl, R²⁸-substituted or unsubstituted heteroalkyl, R²⁸-substituted or unsubstituted cycloalkyl, R²⁸-substituted or unsubstituted heterocycloalkyl, R²⁸-substituted or unsubstituted aryl, or R²⁸-substituted or unsubstituted heteroaryl. Two adjacent R²⁷ substituents may optionally be joined to form a substituted (e.g. R²⁸-substituted) or unsubstituted cycloalkyl, substituted (e.g. R²⁸-substituted) or unsubstituted heterocycloalkyl, substituted (e.g. R²⁸-substituted) or unsubstituted aryl, or substituted (e.g. R²⁸-substituted) or unsubstituted heteroaryl.

In some embodiments of the compounds provided herein, R⁴ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —C(O)CH₃, —NHC═(O)NH₂, —NHSO₂H, —NHC═(O)H, —NHC(O)—OH, —NHOH, —OCF₃, —OCHF₂, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl. In some embodiments, two adjacent R⁴ substituents may optionally be joined to form a substituted (e.g. R²⁹-substituted) or unsubstituted cycloalkyl, substituted (e.g. R²⁹-substituted) or unsubstituted heterocycloalkyl, substituted (e.g. R²⁹-substituted) or unsubstituted aryl, or substituted (e.g. R²⁹-substituted) or unsubstituted heteroaryl.

R²⁹ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³⁰-substituted or unsubstituted alkyl, R³⁰-substituted or unsubstituted heteroalkyl, R³⁰-substituted or unsubstituted cycloalkyl, R³⁰ substituted or unsubstituted heterocycloalkyl, R³⁰-substituted or unsubstituted aryl, or R³⁰-substituted or unsubstituted heteroaryl. Two adjacent R²⁹ substituents may optionally be joined to form a substituted (e.g. R³⁰-substituted) or unsubstituted cycloalkyl, substituted (e.g. R³⁰-substituted) or unsubstituted heterocycloalkyl, substituted (e.g. R³⁰-substituted) or unsubstituted aryl, or substituted (e.g. R³⁰-substituted) or unsubstituted heteroaryl.

R³⁰ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl. Two adjacent R³⁰ substituents may optionally be joined to form a substituted (e.g. R³¹-substituted) or unsubstituted cycloalkyl, substituted (e.g. R³¹-substituted) or unsubstituted heterocycloalkyl, substituted (e.g. R³¹-substituted) or unsubstituted aryl, or substituted (e.g. R³¹-substituted) or unsubstituted heteroaryl.

In some embodiments of the compounds provided herein, R⁵ is hydrogen, halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³²-substituted or unsubstituted alkyl, R³²-substituted or unsubstituted heteroalkyl, R³²-substituted or unsubstituted cycloalkyl, R³²-substituted or unsubstituted heterocycloalkyl, R³²-substituted or unsubstituted aryl, or R³²-substituted or unsubstituted heteroaryl. In some embodiments, two adjacent R⁵ substituents may optionally be joined to form a substituted (e.g. R³²-substituted) or unsubstituted cycloalkyl, substituted (e.g. R³²-substituted) or unsubstituted heterocycloalkyl, substituted (e.g. R³²-substituted) or unsubstituted aryl, or substituted (e.g. R³²-substituted) or unsubstituted heteroaryl.

R³² is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³³-substituted or unsubstituted alkyl, R³³-substituted or unsubstituted heteroalkyl, R³³-substituted or unsubstituted cycloalkyl, R³³ substituted or unsubstituted heterocycloalkyl, R³³-substituted or unsubstituted aryl, or R³³-substituted or unsubstituted heteroaryl. Two adjacent R³² substituents may optionally be joined to form a substituted (e.g. R³³-substituted) or unsubstituted cycloalkyl, substituted (e.g. R³³-substituted) or unsubstituted heterocycloalkyl, substituted (e.g. R³³-substituted) or unsubstituted aryl, or substituted (e.g. R³³-substituted) or unsubstituted heteroaryl.

R³³ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl. Two adjacent R³³ substituents may optionally be joined to form a substituted (e.g. R³⁴-substituted) or unsubstituted cycloalkyl, substituted (e.g. R³⁴-substituted) or unsubstituted heterocycloalkyl, substituted (e.g. R³⁴-substituted) or unsubstituted aryl, or substituted (e.g. R³⁴-substituted) or unsubstituted heteroaryl.

In some embodiments of the compounds provided herein, R⁶ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³⁵-substituted or unsubstituted alkyl, R³⁵-substituted or unsubstituted heteroalkyl, R³⁵-substituted or unsubstituted cycloalkyl, R³⁵-substituted or unsubstituted heterocycloalkyl, R³⁵-substituted or unsubstituted aryl, or R³⁵-substituted or unsubstituted heteroaryl. In some embodiments, two adjacent R⁶ substituents may optionally be joined to form a substituted (e.g. R³⁵-substituted) or unsubstituted cycloalkyl, substituted (e.g. R³⁵-substituted) or unsubstituted heterocycloalkyl, substituted (e.g. R³⁵-substituted) or unsubstituted aryl, or substituted (e.g. R³⁵-substituted) or unsubstituted heteroaryl.

R³⁵ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³⁶-substituted or unsubstituted alkyl, R³⁶-substituted or unsubstituted heteroalkyl, R³⁶-substituted or unsubstituted cycloalkyl, R³⁶ substituted or unsubstituted heterocycloalkyl, R³⁶-substituted or unsubstituted aryl, or R³⁶-substituted or unsubstituted heteroaryl. Two adjacent R³⁵ substituents may optionally be joined to form a substituted (e.g. R³⁶-substituted) or unsubstituted cycloalkyl, substituted (e.g. R³⁶-substituted) or unsubstituted heterocycloalkyl, substituted (e.g. R³⁶-substituted) or unsubstituted aryl, or substituted (e.g. R³⁶-substituted) or unsubstituted heteroaryl.

R³⁶ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³⁷-substituted or unsubstituted alkyl, R³⁷-substituted or unsubstituted heteroalkyl, R³⁷-substituted or unsubstituted cycloalkyl, R³⁷-substituted or unsubstituted heterocycloalkyl, R³⁷-substituted or unsubstituted aryl, or R³⁷-substituted or unsubstituted heteroaryl. Two adjacent R³⁶ substituents may optionally be joined to form a substituted (e.g. R³⁷-substituted) or unsubstituted cycloalkyl, substituted (e.g. R³⁷-substituted) or unsubstituted heterocycloalkyl, substituted (e.g. R³⁷-substituted) or unsubstituted aryl, or substituted (e.g. R³⁷-substituted) or unsubstituted heteroaryl.

In some embodiments of the compounds provided herein, R⁷ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³⁸-substituted or unsubstituted alkyl, R³⁸-substituted or unsubstituted heteroalkyl, R³⁸-substituted or unsubstituted cycloalkyl, R³⁸-substituted or unsubstituted heterocycloalkyl, R³⁸-substituted or unsubstituted aryl, or R³⁸-substituted or unsubstituted heteroaryl.

R³⁸ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R³⁹-substituted or unsubstituted alkyl, R³⁹-substituted or unsubstituted heteroalkyl, R³⁹-substituted or unsubstituted cycloalkyl, R³⁹ substituted or unsubstituted heterocycloalkyl, R³⁹-substituted or unsubstituted aryl, or R³⁹-substituted or unsubstituted heteroaryl.

R³⁹ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴⁰-substituted or unsubstituted alkyl, R⁴⁰-substituted or unsubstituted heteroalkyl, R⁴⁰-substituted or unsubstituted cycloalkyl, R⁴⁰-substituted or unsubstituted heterocycloalkyl, R⁴⁰-substituted or unsubstituted aryl, or R⁴⁰-substituted or unsubstituted heteroaryl.

In some embodiments of the compounds provided herein, R⁸ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴¹-substituted or unsubstituted alkyl, R⁴¹-substituted or unsubstituted heteroalkyl, R⁴¹-substituted or unsubstituted cycloalkyl, R⁴¹-substituted or unsubstituted heterocycloalkyl, R⁴¹-substituted or unsubstituted aryl, or R⁴¹-substituted or unsubstituted heteroaryl.

R⁴¹ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴²-substituted or unsubstituted alkyl, R⁴²-substituted or unsubstituted heteroalkyl, R⁴²-substituted or unsubstituted cycloalkyl, esubstituted or unsubstituted heterocycloalkyl, R⁴²-substituted or unsubstituted aryl, or R⁴²-substituted or unsubstituted heteroaryl.

R⁴² is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴³-substituted or unsubstituted alkyl, R⁴³-substituted or unsubstituted heteroalkyl, R⁴³-substituted or unsubstituted cycloalkyl, R⁴³-substituted or unsubstituted heterocycloalkyl, R⁴³-substituted or unsubstituted aryl, or R⁴³— substituted or unsubstituted heteroaryl.

In some embodiments of the compounds provided herein, R⁹ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴⁴-substituted or unsubstituted alkyl, R⁴⁴-substituted or unsubstituted heteroalkyl, R⁴⁴-substituted or unsubstituted cycloalkyl, R⁴⁴-substituted or unsubstituted heterocycloalkyl, R⁴⁴-substituted or unsubstituted aryl, or R⁴⁴-substituted or unsubstituted heteroaryl.

R⁴⁴ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴⁵-substituted or unsubstituted alkyl, R⁴⁵-substituted or unsubstituted heteroalkyl, R⁴⁵-substituted or unsubstituted cycloalkyl, esubstituted or unsubstituted heterocycloalkyl, R⁴⁵-substituted or unsubstituted aryl, or R⁴⁵-substituted or unsubstituted heteroaryl.

R⁴⁵ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴⁶-substituted or unsubstituted alkyl, R⁴⁶-substituted or unsubstituted heteroalkyl, R⁴⁶-substituted or unsubstituted cycloalkyl, R⁴⁶-substituted or unsubstituted heterocycloalkyl, R⁴⁶-substituted or unsubstituted aryl, or R⁴⁶-substituted or unsubstituted heteroaryl.

In some embodiments of the compounds provided herein, R¹⁰ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴⁷-substituted or unsubstituted alkyl, R⁴⁷-substituted or unsubstituted heteroalkyl, R⁴⁷-substituted or unsubstituted cycloalkyl, R⁴⁷-substituted or unsubstituted heterocycloalkyl, R⁴⁷-substituted or unsubstituted aryl, or R⁴⁷-substituted or unsubstituted heteroaryl.

R⁴⁷ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴⁸-substituted or unsubstituted alkyl, R⁴⁸-substituted or unsubstituted heteroalkyl, R⁴⁸-substituted or unsubstituted cycloalkyl, R⁴⁸ substituted or unsubstituted heterocycloalkyl, R⁴⁸-substituted or unsubstituted aryl, or R⁴⁸-substituted or unsubstituted heteroaryl.

R⁴⁸ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁴⁹-substituted or unsubstituted alkyl, R⁴⁹-substituted or unsubstituted heteroalkyl, R⁴⁹-substituted or unsubstituted cycloalkyl, R⁴⁹-substituted or unsubstituted heterocycloalkyl, R⁴⁹-substituted or unsubstituted aryl, or R⁴⁹-substituted or unsubstituted heteroaryl.

In some embodiments of the compounds provided herein, R¹¹ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵⁰-substituted or unsubstituted alkyl, R⁵⁰-substituted or unsubstituted heteroalkyl, R⁵⁰-substituted or unsubstituted cycloalkyl, R⁵⁰-substituted or unsubstituted heterocycloalkyl, R⁵⁰-substituted or unsubstituted aryl, or R⁵⁰-substituted or unsubstituted heteroaryl. In some embodiments, two adjacent R¹¹ substituents may optionally be joined to form a substituted (e.g. R⁵⁰-substituted) or unsubstituted cycloalkyl, substituted (e.g. R⁵⁰-substituted) or unsubstituted heterocycloalkyl, substituted (e.g. R⁵⁰-substituted) or unsubstituted aryl, or substituted (e.g. R⁵⁰-substituted) or unsubstituted heteroaryl.

R⁵⁰ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵¹-substituted or unsubstituted alkyl, R⁵¹-substituted or unsubstituted heteroalkyl, R⁵¹-substituted or unsubstituted cycloalkyl, R⁵¹ substituted or unsubstituted heterocycloalkyl, R⁵¹-substituted or unsubstituted aryl, or R⁵¹-substituted or unsubstituted heteroaryl. Two adjacent R⁵⁰ substituents may optionally be joined to form a substituted (e.g. R⁵¹-substituted) or unsubstituted cycloalkyl, substituted (e.g. R⁵¹-substituted) or unsubstituted heterocycloalkyl, substituted (e.g. R⁵¹-substituted) or unsubstituted aryl, or substituted (e.g. R⁵¹-substituted) or unsubstituted heteroaryl.

R⁵¹ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵²-substituted or unsubstituted alkyl, R⁵²-substituted or unsubstituted heteroalkyl, R⁵²-substituted or unsubstituted cycloalkyl, R⁵²-substituted or unsubstituted heterocycloalkyl, R⁵²-substituted or unsubstituted aryl, or R⁵²-substituted or unsubstituted heteroaryl. Two adjacent R⁵¹ substituents may optionally be joined to form a substituted (e.g. R⁵²-substituted) or unsubstituted cycloalkyl, substituted (e.g. R⁵²-substituted) or unsubstituted heterocycloalkyl, substituted (e.g. R⁵²-substituted) or unsubstituted aryl, or substituted (e.g. R⁵²-substituted) or unsubstituted heteroaryl.

In some embodiments of the compounds provided herein, R¹² is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵³-substituted or unsubstituted alkyl, R⁵³-substituted or unsubstituted heteroalkyl, R⁵³-substituted or unsubstituted cycloalkyl, R⁵³-substituted or unsubstituted heterocycloalkyl, R⁵³-substituted or unsubstituted aryl, or R⁵³-substituted or unsubstituted heteroaryl. In some embodiments, two adjacent R¹² substituents may optionally be joined to form a substituted (e.g. R⁵³-substituted) or unsubstituted cycloalkyl, substituted (e.g. R⁵³-substituted) or unsubstituted heterocycloalkyl, substituted (e.g. R⁵³-substituted) or unsubstituted aryl, or substituted (e.g. R⁵³-substituted) or unsubstituted heteroaryl.

R⁵³ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵⁴-substituted or unsubstituted alkyl, R⁵⁴-substituted or unsubstituted heteroalkyl, R⁵⁴-substituted or unsubstituted cycloalkyl, R⁵⁴ substituted or unsubstituted heterocycloalkyl, R⁵⁴-substituted or unsubstituted aryl, or R⁵⁴-substituted or unsubstituted heteroaryl. Two adjacent R⁵³ substituents may optionally be joined to form a substituted (e.g. R⁵⁴-substituted) or unsubstituted cycloalkyl, substituted (e.g. R⁵⁴-substituted) or unsubstituted heterocycloalkyl, substituted (e.g. R⁵⁴-substituted) or unsubstituted aryl, or substituted (e.g. R⁵⁴-substituted) or unsubstituted heteroaryl.

R⁵⁴ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵⁵-substituted or unsubstituted alkyl, R⁵⁵-substituted or unsubstituted heteroalkyl, R⁵⁵-substituted or unsubstituted cycloalkyl, R⁵⁵-substituted or unsubstituted heterocycloalkyl, R⁵⁵-substituted or unsubstituted aryl, or R⁵⁵-substituted or unsubstituted heteroaryl. Two adjacent R⁵⁴ substituents may optionally be joined to form a substituted (e.g. R⁵⁵-substituted) or unsubstituted cycloalkyl, substituted (e.g. R⁵⁵-substituted) or unsubstituted heterocycloalkyl, substituted (e.g. R⁵⁵-substituted) or unsubstituted aryl, or substituted (e.g. R⁵⁵-substituted) or unsubstituted heteroaryl.

In some embodiments of the compounds provided herein, R¹³ is hydrogen, oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵⁶-substituted or unsubstituted alkyl, R⁵⁶-substituted or unsubstituted heteroalkyl, R⁵⁶-substituted or unsubstituted cycloalkyl, R⁵⁶-substituted or unsubstituted heterocycloalkyl, R⁵⁶-substituted or unsubstituted aryl, or R⁵⁶-substituted or unsubstituted heteroaryl. In some embodiments, two adjacent R¹³ substituents may optionally be joined to form a substituted (e.g. R⁵⁶-substituted) or unsubstituted cycloalkyl, substituted (e.g. R⁵⁶-substituted) or unsubstituted heterocycloalkyl, substituted (e.g. R⁵⁶-substituted) or unsubstituted aryl, or substituted (e.g. R⁵⁶-substituted) or unsubstituted heteroaryl.

R⁵⁶ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵⁷-substituted or unsubstituted alkyl, R⁵⁷-substituted or unsubstituted heteroalkyl, R⁵⁷-substituted or unsubstituted cycloalkyl, R⁵⁷ substituted or unsubstituted heterocycloalkyl, R⁵⁷-substituted or unsubstituted aryl, or R⁵⁷-substituted or unsubstituted heteroaryl. Two adjacent R⁵⁶ substituents may optionally be joined to form a substituted (e.g. R⁵⁷-substituted) or unsubstituted cycloalkyl, substituted (e.g. R⁵⁷-substituted) or unsubstituted heterocycloalkyl, substituted (e.g. R⁵⁷-substituted) or unsubstituted aryl, or substituted (e.g. R⁵⁷-substituted) or unsubstituted heteroaryl.

R⁵⁷ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵⁸-substituted or unsubstituted alkyl, R⁵⁸-substituted or unsubstituted heteroalkyl, R⁵⁸-substituted or unsubstituted cycloalkyl, R⁵⁸-substituted or unsubstituted heterocycloalkyl, R⁵⁸-substituted or unsubstituted aryl, or R⁵⁸-substituted or unsubstituted heteroaryl. Two adjacent R⁵⁷ substituents may optionally be joined to form a substituted (e.g. R⁵⁸-substituted) or unsubstituted cycloalkyl, substituted (e.g. R⁵⁸-substituted) or unsubstituted heterocycloalkyl, substituted (e.g. R⁵⁸-substituted) or unsubstituted aryl, or substituted (e.g. R⁵⁸-substituted) or unsubstituted heteroaryl.

In another embodiment of the compounds provided herein, R¹⁴ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁵⁹-substituted or unsubstituted alkyl, R⁵⁹-substituted or unsubstituted heteroalkyl, R⁵⁹-substituted or unsubstituted cycloalkyl, R⁵⁹-substituted or unsubstituted heterocycloalkyl, R⁵⁹-substituted or unsubstituted aryl, or R⁵⁹-substituted or unsubstituted heteroaryl.

R⁵⁹ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, R⁶⁰-substituted or unsubstituted alkyl, R⁶⁰-substituted or unsubstituted heteroalkyl, R⁶⁰-substituted or unsubstituted cycloalkyl, R⁶⁰-substituted or unsubstituted heterocycloalkyl, R⁶⁰-substituted or unsubstituted aryl, or R⁶⁰-substituted or unsubstituted heteroaryl.

R⁶⁰ independently is halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶¹-substituted or unsubstituted alkyl, R⁶¹-substituted or unsubstituted heteroalkyl, R⁶¹-substituted or unsubstituted cycloalkyl, R⁶¹-substituted or unsubstituted heterocycloalkyl, R⁶¹-substituted or unsubstituted aryl, or R⁶¹-substituted or unsubstituted heteroaryl.

In a further embodiment of the compounds provided herein, R¹⁵ is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶²-substituted or unsubstituted alkyl, R⁶²-substituted or unsubstituted heteroalkyl, R⁶²-substituted or unsubstituted cycloalkyl, R⁶²-substituted or unsubstituted heterocycloalkyl, R⁶²-substituted or unsubstituted aryl, or R⁶²-substituted or unsubstituted heteroaryl.

R⁶² is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶³-substituted or unsubstituted alkyl, R⁶³-substituted or unsubstituted heteroalkyl, R⁶³-substituted or unsubstituted cycloalkyl, R⁶³-substituted or unsubstituted heterocycloalkyl, R⁶³-substituted or unsubstituted aryl, or R⁶³-substituted or unsubstituted heteroaryl.

R⁶³ independently is halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶⁴-substituted or unsubstituted alkyl, R⁶⁴-substituted or unsubstituted heteroalkyl, R⁶⁴-substituted or unsubstituted cycloalkyl, R⁶⁴-substituted or unsubstituted heterocycloalkyl, R⁶⁴-substituted or unsubstituted aryl, or R⁶⁴-substituted or unsubstituted heteroaryl.

In some embodiments of the compounds provided herein, R¹⁶ is independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶⁵-substituted or unsubstituted alkyl, R⁶⁵-substituted or unsubstituted heteroalkyl, R⁶⁵-substituted or unsubstituted cycloalkyl, R⁶⁵-substituted or unsubstituted heterocycloalkyl, R⁶⁵-substituted or unsubstituted aryl, or R⁶⁵-substituted or unsubstituted heteroaryl.

R⁶⁵ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶⁶-substituted or unsubstituted alkyl, R⁶⁶-substituted or unsubstituted heteroalkyl, R⁶⁶-substituted or unsubstituted cycloalkyl, R⁶⁶-substituted or unsubstituted heterocycloalkyl, R⁶⁶-substituted or unsubstituted aryl, or R⁶⁶-substituted or unsubstituted heteroaryl.

R⁶⁶ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶⁷-substituted or unsubstituted alkyl, R⁶⁷-substituted or unsubstituted heteroalkyl, R⁶⁷-substituted or unsubstituted cycloalkyl, R⁶⁷-substituted or unsubstituted heterocycloalkyl, R⁶⁷-substituted or unsubstituted aryl, or R⁶⁷-substituted or unsubstituted heteroaryl.

In some embodiments of the compounds provided herein, R¹⁷ is independently hydrogen, halogen, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶⁸-substituted or unsubstituted alkyl, R⁶⁸-substituted or unsubstituted heteroalkyl, R⁶⁸-substituted or unsubstituted cycloalkyl, R⁶⁸-substituted or unsubstituted heterocycloalkyl, R⁶⁸-substituted or unsubstituted aryl, or R⁶⁸-substituted or unsubstituted heteroaryl.

R⁶⁸ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁶⁹-substituted or unsubstituted alkyl, R⁶⁹-substituted or unsubstituted heteroalkyl, R⁶⁹-substituted or unsubstituted cycloalkyl, R⁶⁹-substituted or unsubstituted heterocycloalkyl, R⁶⁹-substituted or unsubstituted aryl, or R⁶⁹-substituted or unsubstituted heteroaryl.

R⁶⁹ is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R⁷⁰-substituted or unsubstituted alkyl, R⁷⁰-substituted or unsubstituted heteroalkyl, R⁷⁰-substituted or unsubstituted cycloalkyl, R⁷⁰-substituted or unsubstituted heterocycloalkyl, R⁷⁰-substituted or unsubstituted aryl, or R⁷⁰-substituted or unsubstituted heteroaryl.

In some embodiments of the compounds provided herein, R^{7b} is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{38b}-substituted or unsubstituted alkyl, R^{38b}-substituted or unsubstituted heteroalkyl, R^{38b}-substituted or unsubstituted cycloalkyl, R^{38b}-substituted or unsubstituted heterocycloalkyl, R^{38b}-substituted or unsubstituted aryl, or R^{38b}-substituted or unsubstituted heteroaryl.

R^{38b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{39b}-substituted or unsubstituted alkyl, R^{39b}-substituted or unsubstituted heteroalkyl, R^{39b}-substituted or unsubstituted cycloalkyl, R^{39b} substituted or unsubstituted heterocycloalkyl, R^{39b}-substituted or unsubstituted aryl, or R^{39b}— substituted or unsubstituted heteroaryl.

R^{39b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{40b}-substituted or unsubstituted alkyl, R^{40b}-substituted or unsubstituted heteroalkyl, R^{40b}-substituted or unsubstituted cycloalkyl, R^{40b}-substituted or unsubstituted heterocycloalkyl, R^{40b}-substituted or unsubstituted aryl, or R^{40b}-substituted or unsubstituted heteroaryl.

In some embodiments of the compounds provided herein, R^{8b} is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{41b}-substituted or unsubstituted alkyl, R^{41b}-substituted or unsubstituted heteroalkyl, R^{41b}-substituted or unsubstituted cycloalkyl, R^{41b}-substituted or unsubstituted heterocycloalkyl, R^{41b}-substituted or unsubstituted aryl, or R^{41b}-substituted or unsubstituted heteroaryl.

R^{41b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{42b}-substituted or unsubstituted alkyl, R^{42b}-substituted or unsubstituted heteroalkyl, R^{42b}-substituted or unsubstituted cycloalkyl, R^{42b} substituted or unsubstituted heterocycloalkyl, R^{42b}-substituted or unsubstituted aryl, or R^{42b}-substituted or unsubstituted heteroaryl.

R^{42b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{43b}-substituted or unsubstituted alkyl, R^{43b}-substituted or unsubstituted heteroalkyl, R^{43b}-substituted or unsubstituted cycloalkyl, R^{43b}-substituted or unsubstituted heterocycloalkyl, R^{43b}-substituted or unsubstituted aryl, or R^{43b}-substituted or unsubstituted heteroaryl.

In some embodiments of the compounds provided herein, R^{9b} is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{44b}-substituted or unsubstituted alkyl, R^{44b}-substituted or unsubstituted heteroalkyl, R^{44b}-substituted or unsubstituted cycloalkyl, R^{44b}-substituted or unsubstituted heterocycloalkyl, R^{44b}-substituted or unsubstituted aryl, or R^{44b}-substituted or unsubstituted heteroaryl.

R^{44b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{45b}-substituted or unsubstituted alkyl, R^{45b}-substituted or unsubstituted heteroalkyl, R^{45b}-substituted or unsubstituted cycloalkyl, R^{45b} substituted or unsubstituted heterocycloalkyl, R^{45b}-substituted or unsubstituted aryl, or R^{45b}-substituted or unsubstituted heteroaryl.

R^{45b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{46b}-substituted or unsubstituted alkyl, R^{46b}-substituted or unsubstituted heteroalkyl, R^{46b}-substituted or unsubstituted cycloalkyl, R^{46b}-substituted or unsubstituted heterocycloalkyl, R^{46b}-substituted or unsubstituted aryl, or R^{46b}-substituted or unsubstituted heteroaryl.

In some embodiments of the compounds provided herein, R^{10b} is hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{47b}-substituted or unsubstituted alkyl, R^{47b}-substituted or unsubstituted heteroalkyl, R^{47b}-substituted or unsubstituted cycloalkyl, R^{47b}-substituted or unsubstituted heterocycloalkyl, R^{47b}-substituted or unsubstituted aryl, or R^{47b}-substituted or unsubstituted heteroaryl.

R^{47b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{48b}-substituted or unsubstituted alkyl, R^{48b}-substituted or unsubstituted heteroalkyl, R^{48b}-substituted or unsubstituted cycloalkyl, R^{48b} substituted or unsubstituted heterocycloalkyl, R^{48b}-substituted or unsubstituted aryl, or R^{48b}-substituted or unsubstituted heteroaryl.

R^{48b} is independently halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{49b}-substituted or unsubstituted alkyl, R^{49b}-substituted or unsubstituted heteroalkyl, R^{49b}-substituted or unsubstituted cycloalkyl, R^{49b}-substituted or unsubstituted heterocycloalkyl, R^{49b}-substituted or unsubstituted aryl, or R^{48b}-substituted or unsubstituted heteroaryl.

In some embodiments of the compounds provided herein, R²², R²⁵, R²⁸, R³¹, R³⁴, R³⁷, R⁴⁰, R⁴³, R⁴⁶, R⁴⁹, R⁵², R⁵⁵, R⁵⁸, R⁶¹, R⁶⁴, R⁶⁷, R⁷⁰, R^{40b}, R^{43b}, R^{46b}, R^{49b}, are independently hydrogen, halogen, oxo, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

In some embodiments, the compound is any one of the compounds described herein or in any table, chart, or figure presented herein.

### III. Methods of Treatment

In another aspect is a method of treating a disease in a patient in need of such treatment, the method including administering a therapeutically effective amount of a compound as provided herein (e.g. formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), or (XLII), including embodiments; a compound of Table 2, 4, 5, 6, 8, 9, 18, 19, 20, 24, 25, 26, 27, 28, FIG. 20, 31, Chart 1, 2, 3, 4, or 5). In some embodiments, the compound is a compound of formula V. In some embodiments, the compound is a compound of formula VI. In a further embodiment, the method of treating a disease includes administering compounds selected from compound having formula (III), (V), (VI), or (VII), including embodiments thereof, and combinations thereof. In another embodiment, the method of treating a disease includes administering compounds selected from compounds having formula (XI), (XVI), (XXI), (XXII), (XXV), (XXVI), (XXVII), (XXVIII), (XXIX), (XXX), (XXXI), (XXXII), (XXXIII), (XXXIV), (XXXV), (XXXVI), (XXXVII), or (XXXIX), including embodiments thereof, or a combination thereof. In some embodiments, the compound is one of the compounds in Table 2, 4, 5, 6, 8, 9, 18, 19, 20, 24, 25, 26, 27, 28, FIG. 20, 31, Chart 1, 2, 3, 4, or 5. In some embodiments, the disease is a neurodegenerative disease. In some embodiments, the disease is a prion disease. In some embodiments, the disease is Creutzfeldt-Jakob disease. In some embodiments, the disease is Alzheimer's disease, Amyotrophic lateral sclerosis, Huntington's disease, or Parkinson's disease. In some embodiments, thethe disease is Alzheimer's disease. In some embodiments, the disease is Amyotrophic lateral sclerosis. In some embodiments, the disease is Huntington's disease. In some embodiments, the disease is Parkinson's disease. In some embodiments, the disease is Bovine spongiform encephalopathy. In some embodiments, the disease is Gerstmann-Strussler-Scheinker syndrome. In some embodiments, the disease is kuru. In some embodiments, the disease is chronic wasting disease. In some embodiments, the disease is scrapie.

In some embodiments, the disease is frontotemporal dementia. In some embodiments, the patient is a human.

In another aspect is a method of decreasing the amount of a prion protein (e.g. Human PrP Uniport P04156 or Mouse PrP Uniport P04925) in a cell, the method including contacting the cell with a compound as provided herein (e.g. formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XOH), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), or (XLII), including embodiments; a compound of Table 2, 4, 5, 6, 8, 9, 18, 19, 20, 24, 25, 26, 27, 28, FIG. 20, 31, Chart 1, 2, 3, 4, or 5). In some embodiments the method of decreasing the amount of prion protein in a cell includes contacting the cell with compound having formula (III), (V), or (VI), including embodiments thereof, and combinations thereof. In a further embodiment the method of decreasing the amount of prio protein in a cell includes contacting the cell with a compound having formula (VII), (XI), (XVI), (XXI), (XXII), (XXV), (XXVI), (XXVII), (XXVIII), (XXIX), (XXX), (XXXI), (XXXII), (XXXIII), (XXXIV), (XXXV), (XXXVI), (XXXVII), (XXXIX), including embodiments thereof, or a combination thereof. In some embodiments, the prion protein is a PrP^{C} protein (e.g. Human PrP Uniport P04156 or Mouse PrP Uniport P04925). In some embodiments, the prion protein is a PrP^{Sc} protein (e.g. Human PrP Uniport P04156 or Mouse PrP Uniport P04925). In some embodiments, the method includes decreasing the amount of a prion protein. In some embodiments, the method includes decreasing the amount of a PrP^{C} protein. In some embodiments, the method includes decreasing the amount of a PrP^{Sc} protein. In some embodiments, the method includes decreasing the production of a prion protein. In some embodiments, the method includes decreasing the production of a PrP^{C} protein. In some embodiments, the method includes decreasing the production of a PrP^{Sc} protein. In some embodiments, the method includes decreasing the amount of an existing prion protein. In some embodiments, the method includes decreasing the amount of existing PrP^{C} protein. In some embodiments, the method includes decreasing the amount of existing PrP^{Sc} protein. In some embodiments, the method includes increasing the degradation of a prion protein. In some embodiments, the method includes increasing the degradation of PrP^{C} protein. In some embodiments, the method includes increasing the degradation of PrP^{Sc} protein. In some embodiments, the method includes reducing the conversion of PrP^{C} to PrP^{Sc}. In some embodiments, the method includes contacting the cell with a compound of formula V. In some embodiments, the method includes contacting the cell with a compound of formula VI. In some embodiments, the method includes contacting the cell with a compound of Table 2, 4, 5, 6, 8, 9, 18, 19, 20, 24, 25, 26, 27, 28, FIG. 20, Chart 1, 2, 3, 4, or 5. In some embodiments of the method, the prion protein is a human prion protein. In some embodiments of the method, the PrP^{C} is human PrP^{C}. In some embodiments of the method, the PrP^{Sc} is human PrP^{Sc}. In some embodiments of the method, the PrP^{C} is bovine PrP^{C}. In some embodiments of the method, the PrP^{Sc} is bovine PrP^{Sc}.

In another aspect is a method of decreasing the amount of amyloid beta includes administering to a patient a compound as provided herein (e.g. formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XOH), (XXIV), (XXV), (XXVI), (XXVII), (XXVIII), (XXIX), (XXX), (XXXI), (XXXII), (XOCH), (XXXIV), (XXXV), (XXXVI), (XXXVII), (XXXVIII), (XXXIX), (XL), (XLI), or (XIII), including embodiments; a compound of Table 2, 4, 5, 6, 8, 9, 18, 19, 20, 24, 25, 26, 27, 28, FIG. 20, 31, Chart 1, 2, 3, 4, or 5). In some embodiments the method of decreasing the amount of amyloid beta includes administering to a patient a compound having formula (III), (V), or (VI), including embodiments thereof, and combinations thereof. In a further embodiment the method of decreasing the amount of amyloid beta includes administering to a patient a compound having formula (VII), (XI), (XVI), (XXI), (XXII), (XXV), (XXVI), (XXVII), (XXVIII), (XXIX), (XXX), (XXXI), (XXXII), (XOCH), (XXXIV), (XXXV), (XXXVI), (XXXVII), (XXXIX), including embodiments thereof, or a combination thereof. In some embodiments, the method includes decreasing the amount of amyloid beta. In some embodiments, the method includes decreasing the amount of amyloid precursor protein. In some embodiments, the method includes decreasing the amount of an amyloid protein. In some embodiments, the method includes decreasing the production of amyloid precursor protein. In some embodiments, the method includes decreasing the production of amyloid beta. In some embodiments, the method includes decreasing the production of an amyloid protein (e.g. amyloid precursor or any fragment thereof). In some embodiments, the method includes decreasing the amount of an existing amyloid protein. In some embodiments, the method includes decreasing the amount of existing amyloid precursor protein. In some embodiments, the method includes decreasing the amount of an existing amyloid precursor protein fragment. In some embodiments, the method includes decreasing the amount of existing amyloid beta. In some embodiments, the method includes increasing the degradation of an amyloid precursor protein fragment. In some embodiments, the method includes increasing the degradation of amyloid precursor protein. In some embodiments, the method includes increasing the degradation of amyloid beta. In some embodiments, the method includes reducing the conversion of amyloid precursor protein to amyloid beta. In some embodiments, the method includes administering a compound of formula V. In some embodiments, the method includes administering a compound of formula VI. In some embodiments, the method includes administering a compound of Table 2, 4, 5, 6, 8, 9, 18, 19, 20, 24, 25, 26, 27, 28, FIG. 20, Chart 1, 2, 3, 4, or 5. In some embodiments of the method, the amyloid precursor protein is human amyloid precursor protein. In some embodiments of the method, the amyloid beta is human amyloid beta.

In some embodiments, any of the methods includes the use of any of the compounds described herein or in any table, chart, or figure presented herein.

### IV. Pharmaceutical Compositions

In another aspect is a pharmaceutical composition including a pharmaceutically acceptable excipient and a compound as provided herein (e.g. formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XOCH), (XXXIV), (XXXV), (XXXVI), (XXXVII), (XXXVIII), (XXXIX), (XL), (XLI), or (XLII), including embodiments; a compound of Table 2, 4, 5, 6, 8, 9, 18, 19, 20, 24, 25, 26, 27, 28, FIG. 20, 31, Chart 1, 2, 3, 4, or 5). In some embodiments, the pharmaceutical composition includes a pharmaceutically acceptable excipient and a compound of formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XOCH), (XXXIV), (XXXV), (XXXVI), (XXXVII), (XXXVIII), (XXXIX), (XL), (XLI), or (XLII). In some embodiments, the pharmaceutical composition includes a pharmaceutically acceptable excipient and a compound of formula V. In some embodiments, the pharmaceutical composition includes a pharmaceutically acceptable excipient and a compound of formula VI. In some embodiments, the compound is a compound having formula (III), (V), or (VI), including embodiments thereof, or a combination thereof. In some embodiments, the compound is a compound having formula (III), (V), or (VI), including embodiments thereof, or a combination thereof, in combination with a pharmaceutically acceptable excipient. In another embodiment, the compound is a compound having formula (VII), (XI), (XVI), (XXI), (XXII), (XXV), (XXVI), (XXVII), (XXVIII), (XXIX), (XXX), (XXXI), (XXXII), (XXXIII), (XXXIV), (XXXV), (XXXVI), (XXXVII), or (XXXIX), including embodiments thereof, or a combination thereof. In another embodiment, the compound is a compound having formula (VII), (XI), (XVI), (XXI), (XXII), (XXV), (XXVI), (XXVII), (XXVIII), (XXIX), (XXX), (XXXI), (XXXII), (XXXIII), (XXXIV), (XXXV), (XXXVI), (XXXVII), or (XXXIX), including embodiments thereof, or a combination thereof, in combination with a pharmaceutically acceptable excipient. In some embodiments, the pharmaceutical compositions include pharmaceutically acceptable salts of the compounds. In certain embodiments, the compounds are covalently attached to a carrier moiety. In certain embodiments, the compounds are non-covalently linked to a carrier moiety.

The pharmaceutical compositions include optical isomers, diastereomers, or pharmaceutically acceptable salts of the modulators disclosed herein. The compound included in the pharmaceutical composition may be covalently attached to a carrier moiety, as described above. Alternatively, the compound included in the pharmaceutical composition is not covalently linked to a carrier moiety.

The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

The compounds of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance, that may also act as diluents, flavoring agents, binders, preservatives, tablet disintegrating agents, or an encapsulating material.

In powders, the carrier is a finely divided solid in a mixture with the finely divided active component (e.g. a compound provided herein. In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. prion protein, amyloid beta, alpha-synuclein, huntingtin), and/or reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., prion disease, protein misfolding disease, Creutzfeldt-Jakob disease, Gerstmann-Strussler-Scheinker syndrome, kuru), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, p. 1, 1975. The exact formulation, route of administration and dosage can be chosen by the individual physician in view of the patient's condition and the particular method in which the compound is used.

### V. Administration

The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The pharmaceutical compositions of the present invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds described herein can be used in combination with one another, with other active agents known to be useful in treating a disease associated with misfolded proteins, prion proteins, or protein aggregates, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In some embodiments, a pharmaceutical composition as described herein includes a compound selected from any of the tables, figures, or charts provided herein.

### EMBODIMENTS

1. A compound having the formula:

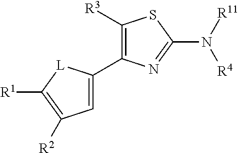

wherein, L is —CR⁶═CH—, —S—, or —O—; R¹ is substituted or unsubstituted cycloalkyl, substituted or unsubstituted heterocycloalkyl, substituted or unsubstituted aryl, or substituted or unsubstituted heteroaryl; R², R³ and R⁶ are independently hydrogen, halogen, —CX₃, —CN, —SO₂Cl, —SOₙR¹⁰, —SOᵥNR⁷R⁸, —NHNH₂, —ONR⁷R⁸, NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ is hydrogen, —C(O)CH₃, substituted or unsubstituted (C₁-C₄)alkyl, substituted or unsubstituted (C₃-C₆)cycloalkyl, or aryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹¹ is substituted or unsubstituted heteroaryl or —C(O)R¹²; R¹² is substituted or unsubstituted cycloalkyl; v is independently 1 or 2; m is independently an integer from 1 to 2; n is independently an integer from 0 to 4; X is independently —Cl, —Br, —I, or —F.

2. The compound of embodiment 1, wherein R¹² is unsubstituted cycloalkyl.

3. The compound of any one of embodiments 1 or 2, wherein R¹² is unsubstituted cyclopropyl.

4. The compound of any one of embodiments 1 to 3, wherein R¹¹ is substituted or unsubstituted heteroaryl.

5. The compound of any one of embodiments 1 to 4 having the formula:

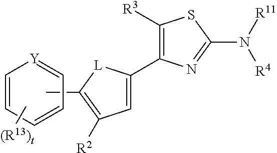

wherein, R¹³ is independently hydrogen, halogen, —CX₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —C(O)R¹⁶ C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁶ and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂-substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; t is independently an integer from 0 to 5; p is independently 1 or 2; q is independently an integer from 1 to 2; r is independently an integer from 0 to 4; X is independently —Cl, —Br, —I, or —F; Y is independently —N═ or —N⁺(O⁻)—.

6. The compound of any one of embodiments 1 to 5 having the formula:

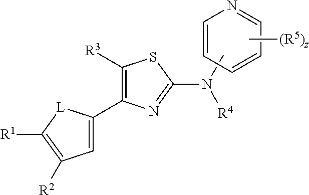

wherein, R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl; R^{7b}, R^{8b}, R^{9b}, and R^{10b} are independently hydrogen, halogen, —CF₃₅—CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; z is independently an integer from 0 to 4; Y1 is independently 1 or 2; m1 is independently an integer from 1 to 2; n1 is independently an integer from 0 to 4; X^{b} is independently —Cl, —Br, —I, or —F.

7. The compound of one of embodiments 1 to 6, wherein L is —CR⁶═CH—.

8. The compound of one of embodiments 1 to 7, wherein R⁶ is hydrogen, —CN, or —OR¹⁰.

9. The compound of one of embodiments 1 to 8, wherein R⁶ is hydrogen.

10. The compound of one of embodiments 1 to 9, wherein L is —S—.

11. The compound of one of embodiments 1 to 10, wherein L is —O—.

12. The compound of one of embodiments 1 to 11 wherein R² is hydrogen.

13. The compound of one of embodiments 1 to 12, wherein R¹ is substituted or unsubstituted aryl or substituted or unsubstituted heteroaryl;

14. The compound of one of embodiments 1 to 13, wherein R¹ is substituted or unsubstituted aryl.

15. The compound of one of embodiments 1 to 14, wherein R¹ is unsubstituted aryl.

16. The compound of one of embodiments 1 to 15, wherein R¹ is unsubstituted phenyl.

17. The compound of one of embodiments 1 to 16 wherein R¹ is substituted or unsubstituted heteroaryl.

18. The compound of one of embodiments 1 to 17, wherein R¹ is unsubstituted heteroaryl.

19. The compound of one of embodiments 1 to 18, wherein R¹ is unsubstituted pyridyl.

20. The compound of one of embodiments 1 to 19, wherein R¹ is 2-pyridyl.

21. The compound of one of embodiments 1 to 20, wherein R³ is hydrogen or substituted or unsubstituted alkyl.

22. The compound of one of embodiments 1 to 21, wherein R³ is hydrogen.

23. The compound of one of embodiments 1 to 22, wherein R⁴ is hydrogen, substituted or unsubstituted (C₁-C₄)alkyl.

24. The compound of one of embodiments 1 to 23, wherein R⁴ is hydrogen.

25. The compound of one of embodiments 1 to 23 wherein R⁴ is methyl, ethyl, n-propyl, isopropyl, t-butyl, or —CF₃.

26. The compound of any one of one of embodiments 1 to 25, having the formula:

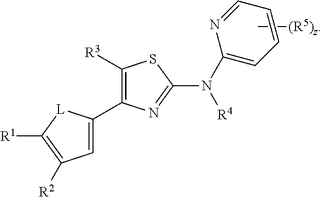

27. The compound of one of embodiments 1 to 26, wherein R⁵ is unsubstituted alkyl or substituted or unsubstituted heteroalkyl.

28. The compound of one of embodiments 1 to 27, wherein R⁵ is unsubstituted alkyl.

29. The compound of one of embodiments 1 to 28, wherein R⁵ is unsubstituted (C₁-C₄)alkyl.

30. The compound of one of embodiments 1 to 29, wherein R⁵ is methyl.

31. The compound of one of embodiments 1 to 27, wherein R⁵ is —OR^{10b} and wherein R^{10b} is substituted or unsubstituted alkyl.

32. The compound of one of embodiments 1 to 31, wherein R^{10b} is substituted or unsubstituted (C₁-C₄)alkyl.

33. The compound of one of embodiments 1 to 32, wherein R^{10b} is methyl.

34. The compound of one of embodiments 1 to 33, wherein z is 1.

35. The compound of one of embodiments 1 to 34, having the formula:

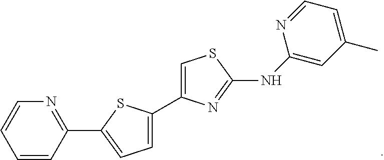 [SMILES: Cc1ccnc(Nc2nc(-c3ccc(-c4ccccn4)s3)cs2)c1]

36. The compound of one of embodiments 1 to 34, having the formula:

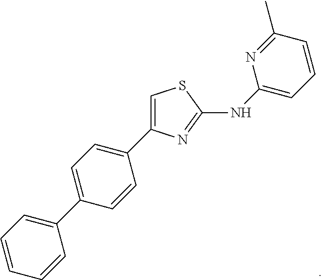 [SMILES: Cc1cccc(Nc2nc(-c3ccc(-c4ccccc4)cc3)cs2)n1]

37. A pharmaceutical composition comprising a pharmaceutically acceptable excipient and a compound of one of embodiments 1 to 36.

38. A method of treating a disease in a patient in need of such treatment, said method comprising administering a therapeutically effective amount of a compound of one of embodiments 1 to 36.

39. The method of embodiment 38, wherein the disease is a neurodegenerative disease.

40. The method of embodiment 39, wherein the disease is a prion disease.

41. The method of embodiment 40, wherein the disease is Creutzfeldt-Jakob disease.

42. The method of embodiment 39, wherein the disease is Alzheimer's disease, Amyotrophic lateral sclerosis, Huntington's disease, or Parkinson's disease.

43. The method of embodiment 42, wherein the disease is Alzheimer's disease.

44. A method of decreasing the amount of a prion protein in a cell, said method comprising contacting said cell with a compound of one of embodiments 1 to 36.

45. A compound having the formula:

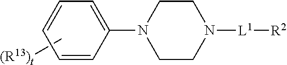

wherein, L¹ is a bond, substituted or unsubstituted alkylene, substituted or unsubstituted heteroalkylene, —NR^{1A}—, —O—, —S—, —C(O)—, or —CHR^{1A}—; R^{1A} and R² are independently hydrogen, halogen, —CX₃, —CN, —SO₂Cl, —SOR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. m, p, q, and v are independently an integer from 1 to 2; n and r are independently an integer from 0 to 4; t is independently an integer from 0 to 5; X and Xa are independently —Cl, —Br, —I, or —F.

46. The compound of embodiment 45 having the formula:

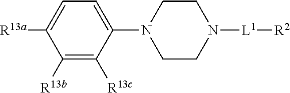

wherein, R^{13a}, R^{13b}, and R^{13c} are independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{13a} and R^{13b} or R^{13b} and R^{13c} may optionally be joined to form a substituted or unsubstituted cycloalkyl, substituted or unsubstituted heterocycloalkyl, substituted or unsubstituted aryl, or substituted or unsubstituted heteroaryl.

47. The compound of one of embodiments 45 to 46 having the formula:

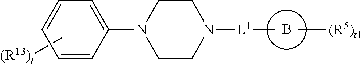

wherein, ring B is substituted or unsubstituted cycloalkyl, substituted or unsubstituted heterocycloalkyl, substituted or unsubstituted aryl, or substituted or unsubstituted heteroaryl; R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl; R^{7b}, R^{8b}, R^{9b}, and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; ml and vl are independently 1 or 2; n1 is independently an integer from 0 to 4; t1 is an integer from 0 to 8; X^{b} is independently —Cl, —Br, —I, or —F.

48. The compound of embodiment 47 having the formula:

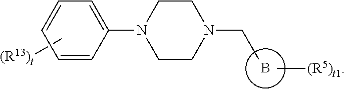

49. The compound of one of embodiments 45 to 48 having the formula:

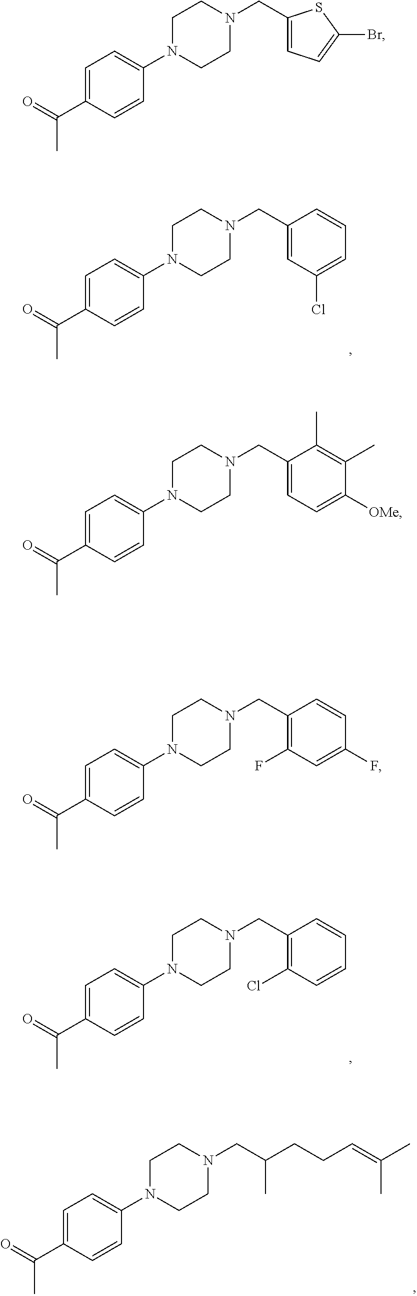 [SMILES: CC(=O)c1ccc(N2CCN(Cc3ccc(Br)s3)CC2)cc1 ; CC(=O)c1ccc(N2CCN(Cc3cccc(Cl)c3)CC2)cc1 ; COc1ccc(CN2CCN(c3ccc(C(C)=O)cc3)CC2)c(C)c1C ; CC(=O)c1ccc(N2CCN(Cc3ccc(F)cc3F)CC2)cc1 ; CC(=O)c1ccc(N2CCN(Cc3ccccc3Cl)CC2)cc1 ; CC(=O)c1ccc(N2CCN(CC(C)CCC=C(C)C)CC2)cc1]

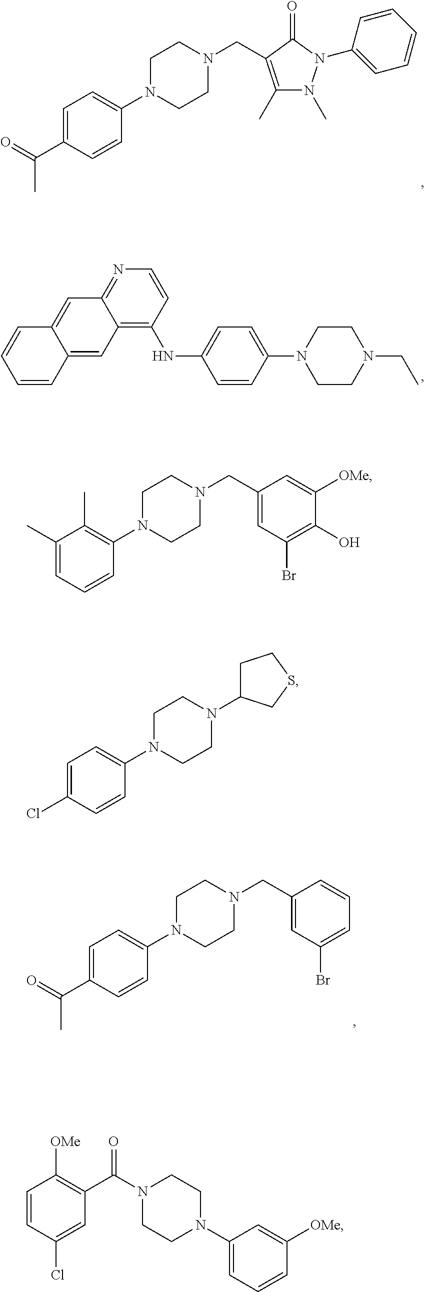

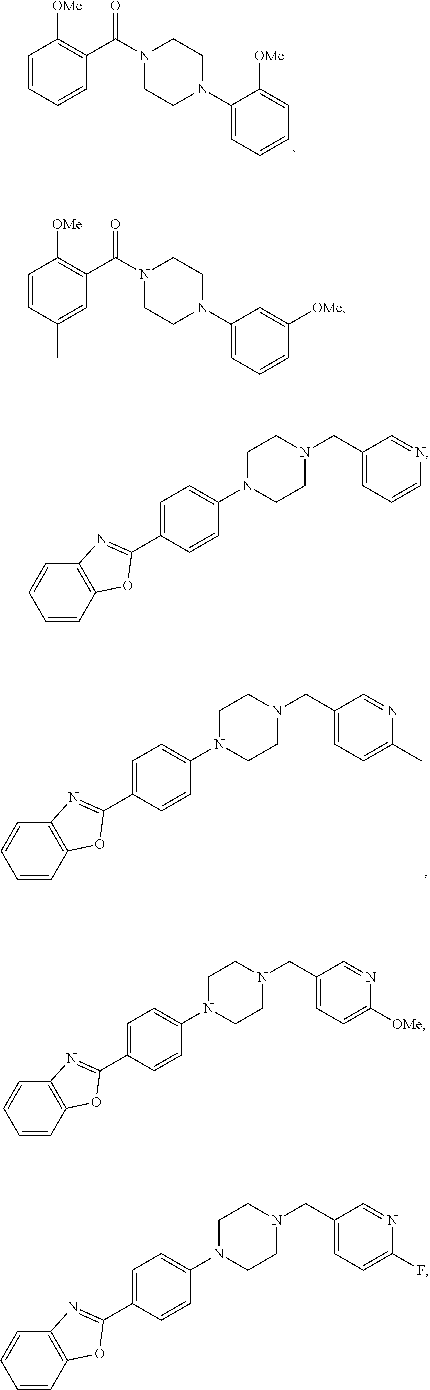 [SMILES: COc1ccccc1C(=O)N1CCN(c2ccccc2OC)CC1 ; COc1cccc(N2CCN(C(=O)c3cc(C)ccc3OC)CC2)c1 ; c1cncc(CN2CCN(c3ccc(-c4nc5ccccc5o4)cc3)CC2)c1 ; Cc1ccc(CN2CCN(c3ccc(-c4nc5ccccc5o4)cc3)CC2)cn1 ; COc1ccc(CN2CCN(c3ccc(-c4nc5ccccc5o4)cc3)CC2)cn1 ; Fc1ccc(CN2CCN(c3ccc(-c4nc5ccccc5o4)cc3)CC2)cn1]

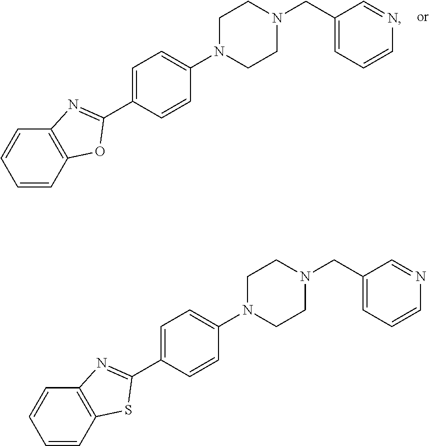 [SMILES: c1cncc(CN2CCN(c3ccc(-c4nc5ccccc5o4)cc3)CC2)c1 ; "or" ; c1cncc(CN2CCN(c3ccc(-c4nc5ccccc5s4)cc3)CC2)c1]

50. A compound having the formula:

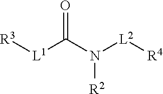

wherein, L¹ is a bond, substituted or unsubstituted alkylene, substituted or unsubstituted heteroalkylene, —NR^{1A}—, —O—, —S—, —C(O)—, or —CHR^{1A}—; L² is a bond, substituted or unsubstituted alkylene, substituted or unsubstituted heteroalkylene, —NR^{1B}—, —O—, —S—, —C(O)—, or —CHR^{1B}—; R^{1A}, R^{1B}, R2, R³, and R⁴ are independently hydrogen, halogen, —CX₃, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

51. The compound of embodiment 50 having the formula:

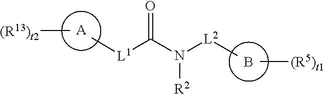

ring A is substituted or unsubstituted cycloalkyl, substituted or unsubstituted heterocycloalkyl, substituted or unsubstituted aryl, or substituted or unsubstituted heteroaryl; ring B is substituted or unsubstituted cycloalkyl, substituted or unsubstituted heterocycloalkyl, substituted or unsubstituted aryl, or substituted or unsubstituted heteroaryl; R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl; R^{7b}R^{8b}, R^{9b}, and R^{10b} are independently hydrogen, halogen, —CF₃, —OH, —NH₂, —COOH, —CONH₂, —NO₂, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂—, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. m1, p, q, and v1 are independently an integer from 1 to 2; n, n1, and r are independently an integer from 0 to 4; t1 is an integer from 0 to 8; t2 is an integer from 0 to 8; X^{a} and X^{b} are independently —Cl, —Br, —I, or —F.

52. The compound of embodiment 51 having the formula:

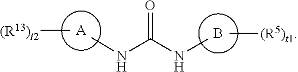

53. The compound of embodiment 51 having the formula:

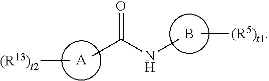

54. The compound of embodiment 51 having the formula:

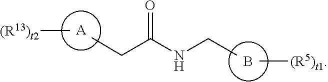

55. The compound of one of embodiments 50 to 54 having the formula:

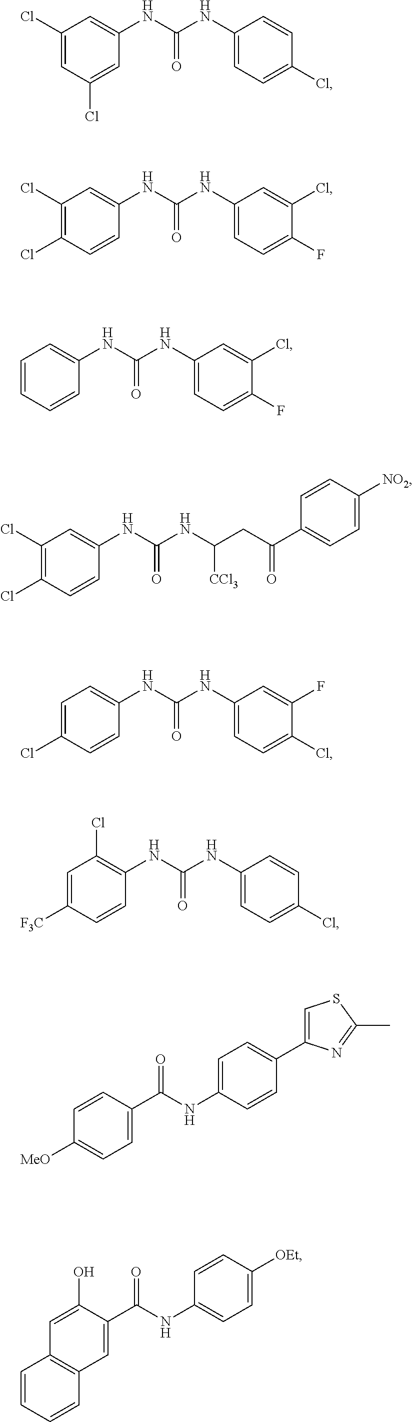

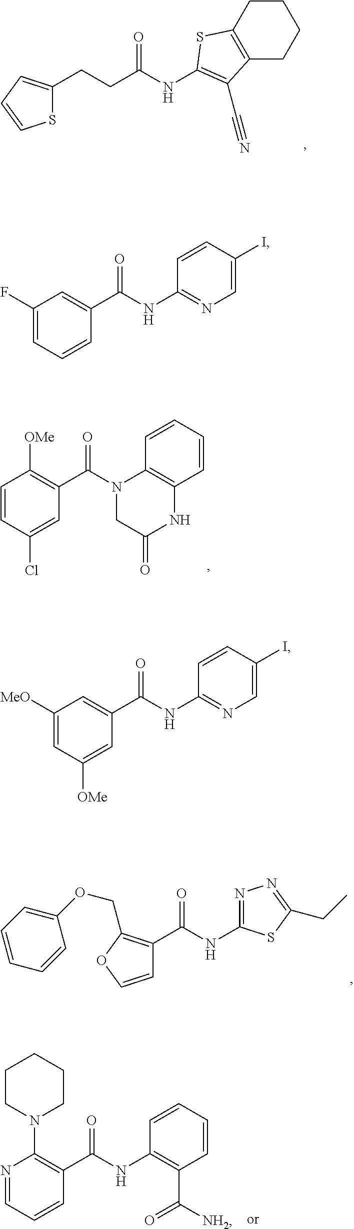

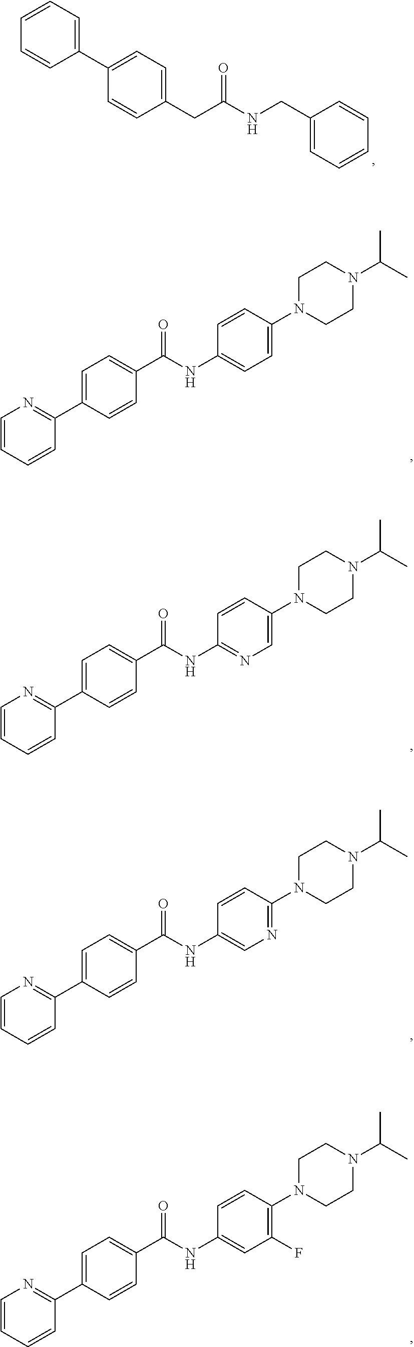 [SMILES: O=C(Cc1ccc(-c2ccccc2)cc1)NCc1ccccc1 ; CC(C)N1CCN(c2ccc(NC(=O)c3ccc(-c4ccccn4)cc3)cc2)CC1 ; CC(C)N1CCN(c2ccc(NC(=O)c3ccc(-c4ccccn4)cc3)nc2)CC1 ; CC(C)N1CCN(c2ccc(NC(=O)c3ccc(-c4ccccn4)cc3)cn2)CC1 ; CC(C)N1CCN(c2ccc(NC(=O)c3ccc(-c4ccccn4)cc3)cc2F)CC1]

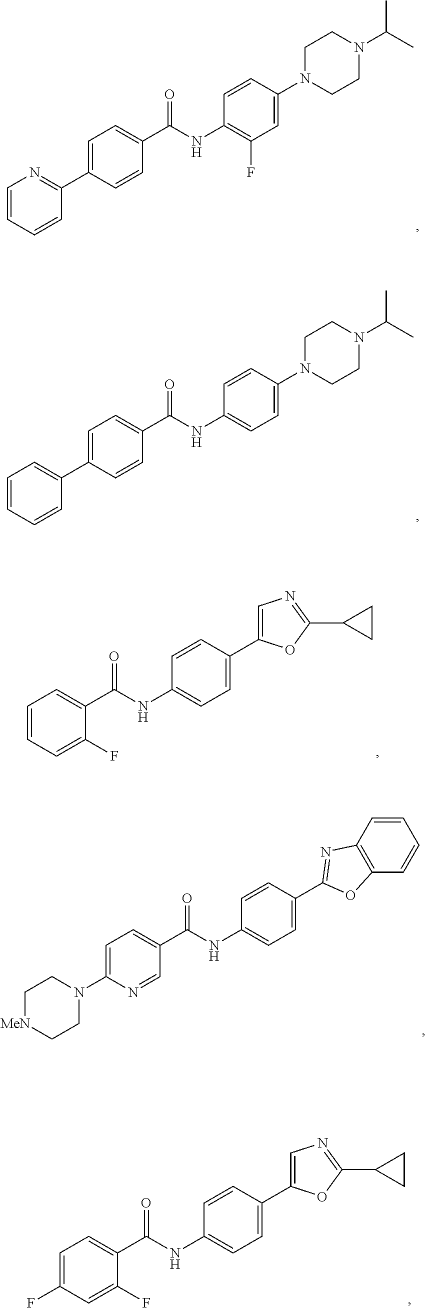 [SMILES: CC(C)N1CCN(c2ccc(NC(=O)c3ccc(-c4ccccn4)cc3)c(F)c2)CC1 ; CC(C)N1CCN(c2ccc(NC(=O)c3ccc(-c4ccccc4)cc3)cc2)CC1 ; O=C(Nc1ccc(-c2cnc(C3CC3)o2)cc1)c1ccccc1F ; CN1CCN(c2ccc(C(=O)Nc3ccc(-c4nc5ccccc5o4)cc3)cn2)CC1 ; O=C(Nc1ccc(-c2cnc(C3CC3)o2)cc1)c1ccc(F)cc1F]

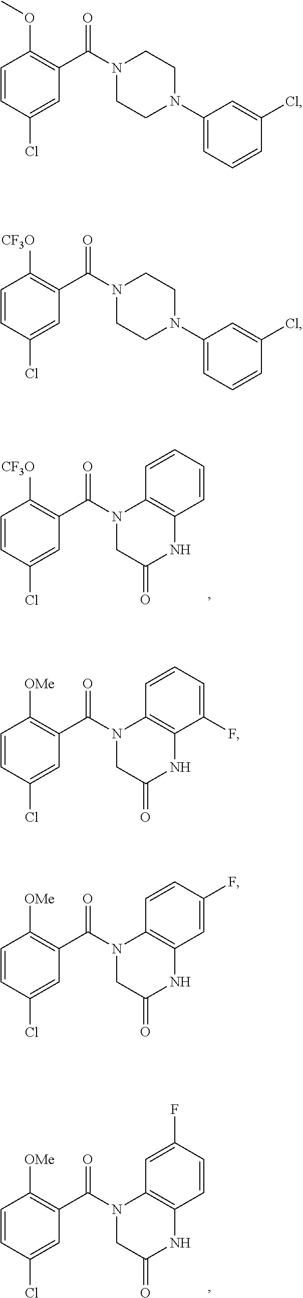

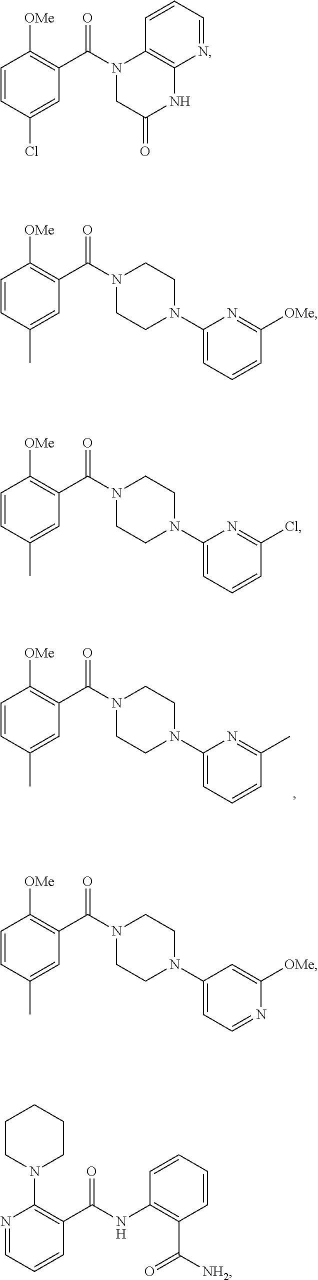 [SMILES: COc1ccc(Cl)cc1C(=O)N1CC(=O)Nc2ncccc21 ; COc1cccc(N2CCN(C(=O)c3cc(C)ccc3OC)CC2)n1 ; COc1ccc(C)cc1C(=O)N1CCN(c2cccc(Cl)n2)CC1 ; COc1ccc(C)cc1C(=O)N1CCN(c2cccc(C)n2)CC1 ; COc1cc(N2CCN(C(=O)c3cc(C)ccc3OC)CC2)ccn1 ; NC(=O)c1ccccc1NC(=O)c1cccnc1N1CCCCC1]

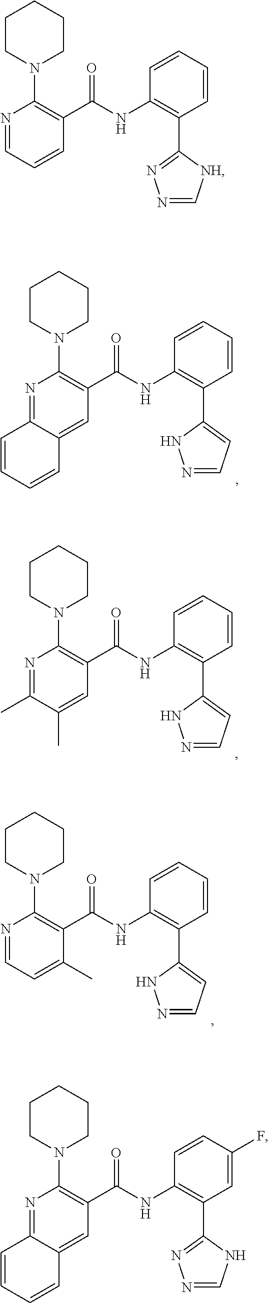 [SMILES: O=C(Nc1ccccc1-c1nnc[nH]1)c1cccnc1N1CCCCC1 ; O=C(Nc1ccccc1-c1ccn[nH]1)c1cc2ccccc2nc1N1CCCCC1 ; Cc1cc(C(=O)Nc2ccccc2-c2ccn[nH]2)c(N2CCCCC2)nc1C ; Cc1ccnc(N2CCCCC2)c1C(=O)Nc1ccccc1-c1ccn[nH]1 ; O=C(Nc1ccc(F)cc1-c1nnc[nH]1)c1cc2ccccc2nc1N1CCCCC1]

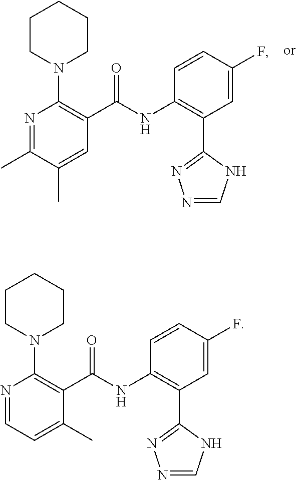 [SMILES: Cc1cc(C(=O)Nc2ccc(F)cc2-c2nnc[nH]2)c(N2CCCCC2)nc1C ; "or" ; Cc1ccnc(N2CCCCC2)c1C(=O)Nc1ccc(F)cc1-c1nnc[nH]1]

56. A compound having the formula:

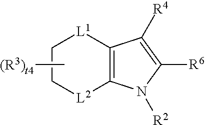

wherein, L¹ is a bond, substituted or unsubstituted alkylene, substituted or unsubstituted heteroalkylene, —NR^{1A}—, —O—, —S—, —C(O)—, or —CHR^{1A}—; L² is a bond, substituted or unsubstituted alkylene, substituted or unsubstituted heteroalkylene, —NR^{1B}—, —O—, —S—, —C(O)—, or —CHR^{1B}—; R^{1A}, R^{1B}, R², R³, R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R³ substituents may optionally be joined to form a substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted heterocycloalkyl or substituted or unsubstituted heteroaryl; R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; t4 is independently 0 to 2; X is independently —Cl, —Br, —I, or —F.

57. The compound of embodiment 56 having the formula:

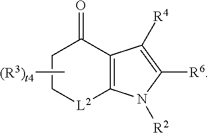

58. The compound of one of embodiments 56 to 57 having the formula:

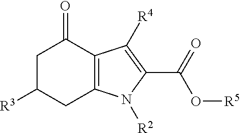

wherein, R⁵ is independently hydrogen, halogen, —CX^{b}₃, —CN, —SO₂Cl, —SOₙ₁R^{10b}, —SOᵥ₁NR^{7b}, R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl; R^{7b}, R^{8b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m1 and v1 are independently an integer from 1 to 2; n1 is independently an integer from 0 to 4; X^{b} is independently —Cl, —Br, —I, or —F.

59. The compound of embodiment 56 having the formula:

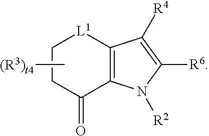

60. The compound of embodiment 56 having the formula:

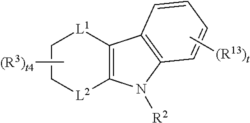

wherein, R¹³ is independently hydrogen, halogen, —CX^{a}₃, —CN, —SO₂Cl, —SOᵣR¹⁷, —SOₚNR¹⁴R¹⁵, —NHNH₂, ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; p and q are independently an integer from 1 to 2; r is independently an integer from 0 to 4; t is an integer from 0 to 4; X^{a} is independently —Cl, —Br, —I, or —F.

61. The compound of one of embodiments 56 to 60 having the formula:

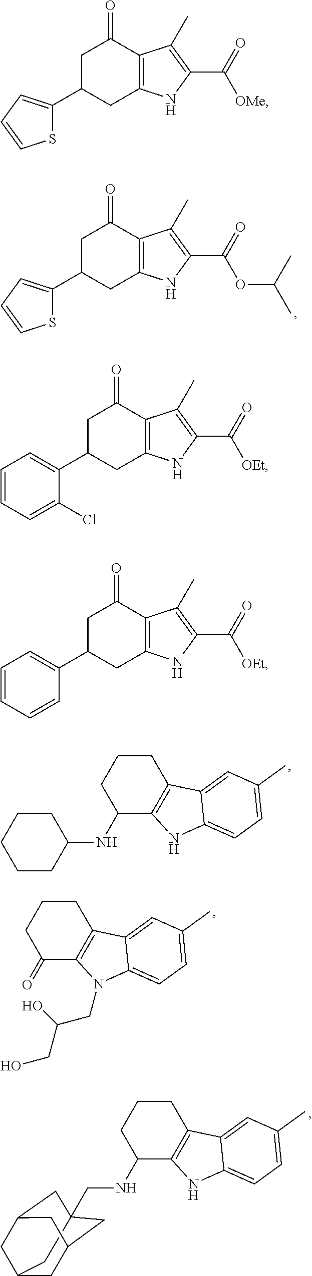

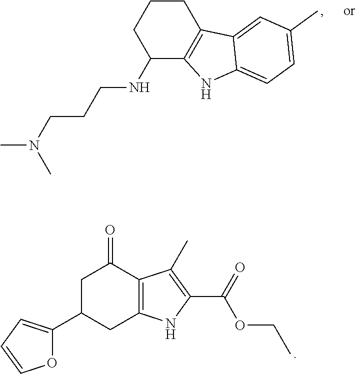

62. A compound having the formula:

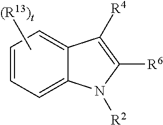

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted heterocycloalkyl or substituted or unsubstituted heteroaryl; R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —C(O)—OR¹⁶, —C(O)NR¹⁴R¹⁵, —OR₁₇, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, p, q, and v are independently an integer from 1 to 2; n and r are independently an integer from 0 to 4; t is an integer from 0 to 4; X and X^{a} are independently —Cl, —Br, —I, or —F.

63. The compound of embodiment 62 having the formula:

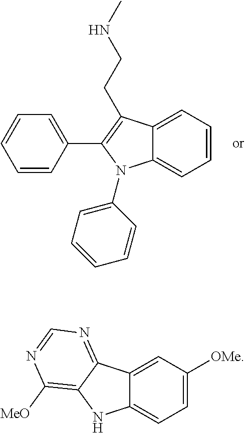 [SMILES: CNCCc1c(-c2ccccc2)n(-c2ccccc2)c2ccccc12 ; COc1ccc2[nH]c3c(OC)ncnc3c2c1]

64. A compound having the formula:

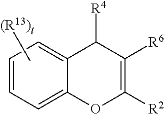

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, p, q, and v are independently an integer from 1 to 2; n and r are independently an integer from 0 to 4; t is an integer from 0 to 4; X and X′ are independently —Cl, —Br, —I, or —F.

65. The compound of embodiment 64 having the formula:

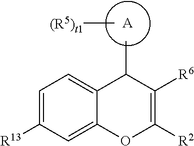

wherein, ring A is substituted or unsubstituted cycloalkyl, substituted or unsubstituted heterocycloalkyl, substituted or unsubstituted aryl, or substituted or unsubstituted heteroaryl; R⁵ is independently hydrogen, halogen, —CX^{b}3, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, R^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl; R^{7b}, R^{8b}, R^{9b}, and R^{19b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m1 and v1 are independently an integer from 1 to 2; n1 is independently an integer from 0 to 4; t1 is an integer from 0 to 8; X^{b} is independently —Cl, —Br, —I, or —F.

66. The compound of one of embodiments 64 to 65 having the formula:

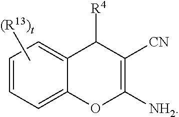

67. The compound of embodiment 64 having the formula:

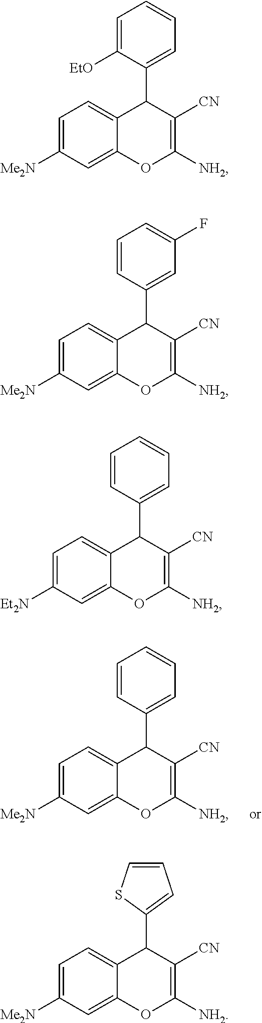

68. A compound having the formula:

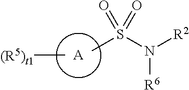

wherein, R² and R⁶ are independently hydrogen, halogen, —CX₃, —CN, —SO₂Cl, —SOₙR¹⁰, —SOᵥNR⁷R⁸, —NHNH₂, ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted heterocycloalkyl or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, p, q, and v are independently an integer from 1 to 2; n and r are independently an integer from 0 to 4; t1 is an integer from 0 to 8; X and X^{a} are independently —Cl, —Br, —I, or —F.

69. The compound of embodiment 68 having the formula:

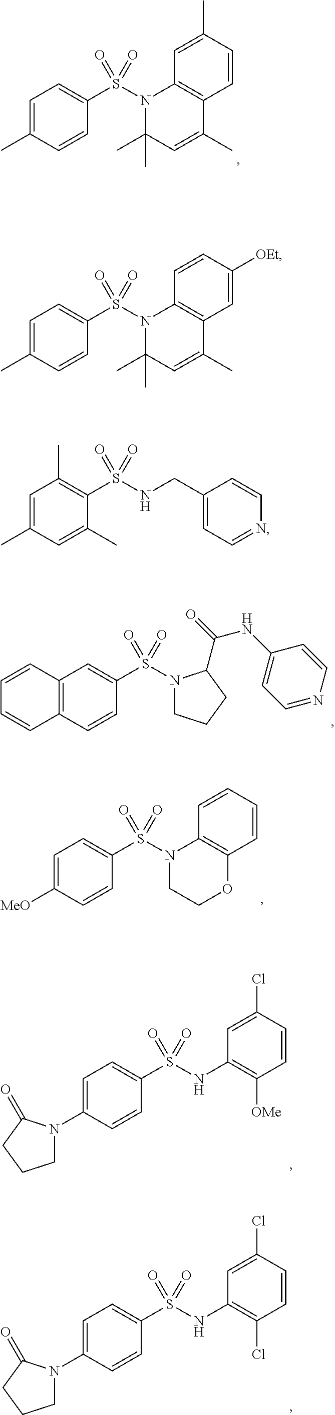

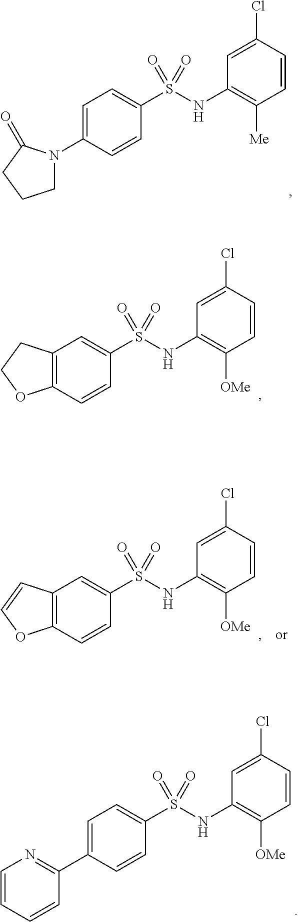 [SMILES: Cc1ccc(Cl)cc1NS(=O)(=O)c1ccc(N2CCCC2=O)cc1 ; COc1ccc(Cl)cc1NS(=O)(=O)c1ccc2c(c1)CCO2 ; COc1ccc(Cl)cc1NS(=O)(=O)c1ccc2occc2c1 ; COc1ccc(Cl)cc1NS(=O)(=O)c1ccc(-c2ccccn2)cc1]

70. A compound having the formula:

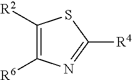

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

71. The compound of embodiment 70 having the formula:

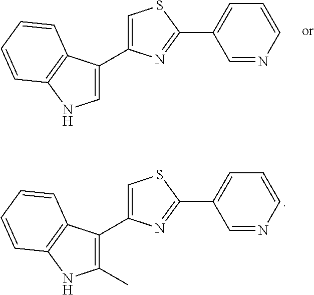 [SMILES: c1cncc(-c2nc(-c3c[nH]c4ccccc34)cs2)c1 ; Cc1[nH]c2ccccc2c1-c1csc(-c2cccnc2)n1]

72. A compound having the formula:

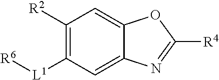

wherein, L¹ is a bond, substituted or unsubstituted alkylene, substituted or unsubstituted heteroalkylene, —NR^{1A}—, —O—, —S—, —C(O)—, or —CHR^{1A}—; R^{1A}, R², R⁴, and R⁶ are independently hydrogen, halogen, —CX₃, —CN, —SO₂Cl, —SOₙR¹⁰, —SOᵥNR⁷R⁸, —NHNH₂, ONR⁷R⁸, —NHC═(O)NHNH₂, —NHC═(O)NR⁷R⁸, —N(O), —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

73. The compound of embodiment 72 having the formula:

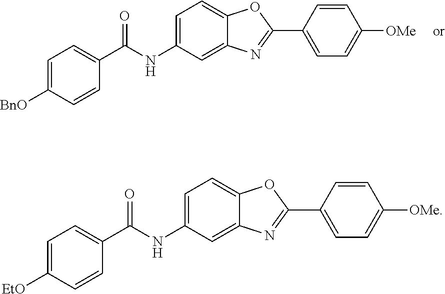 [SMILES: COc1ccc(-c2nc3cc(NC(=O)c4ccc(OCc5ccccc5)cc4)ccc3o2)cc1 ; CCOc1ccc(C(=O)Nc2ccc3oc(-c4ccc(OC)cc4)nc3c2)cc1]

74. A compound having the formula:

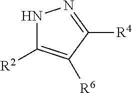

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

75. The compound of embodiment 74 having the formula:

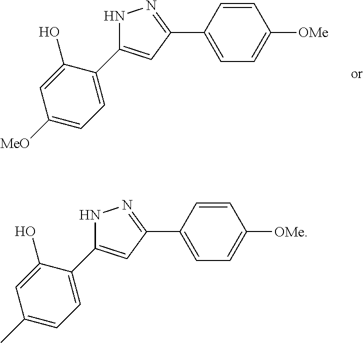 [SMILES: COc1ccc(-c2cc(-c3ccc(OC)cc3O)[nH]n2)cc1 ; COc1ccc(-c2cc(-c3ccc(C)cc3O)[nH]n2)cc1]

76. A compound having the formula:

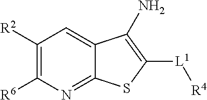

wherein, L¹ is a bond, substituted or unsubstituted alkylene, substituted or unsubstituted heteroalkylene, —NR^{1A}, —O—, —S—, —C(O)—, or —CHR^{1A}—; R^{1A}, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)—NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

77. The compound of embodiment 76 having the formula:

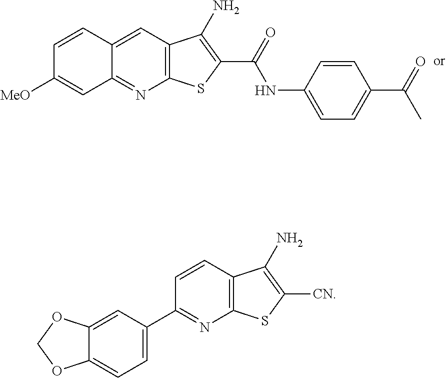 [SMILES: COc1ccc2cc3c(N)c(C(=O)Nc4ccc(C(C)=O)cc4)sc3nc2c1 ; "or" ; N#Cc1sc2nc(-c3ccc4c(c3)OCO4)ccc2c1N]

78. A compound having the formula:

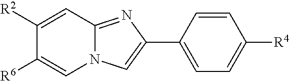

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O), —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

79. The compound of embodiment 78 having the formula:

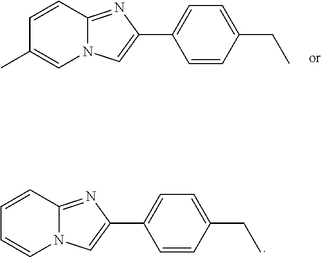 [SMILES: CCc1ccc(-c2cn3cc(C)ccc3n2)cc1 ; "or" ; CCc1ccc(-c2cn3ccccc3n2)cc1]

80. A compound having the formula:

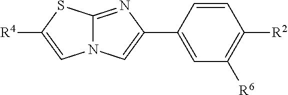

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

81. The compound of embodiment 80 having the formula:

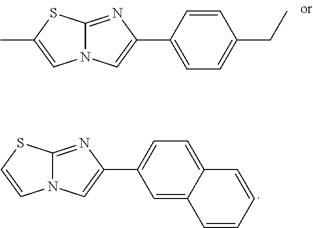 [SMILES: CCc1ccc(-c2cn3cc(C)sc3n2)cc1 ; c1ccc2cc(-c3cn4ccsc4n3)ccc2c1]

82. A compound having the formula:

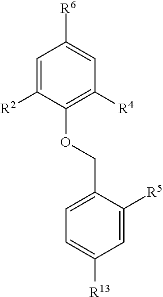

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{7b}, R^{8b}, R^{9b}, and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶ and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, m1, p, q, v, and v1 are independently an integer from 1 to 2; n, n1, and r are independently an integer from 0 to 4; X, Xa and X^{b} are independently —Cl, —Br, —I, or —F.

83. The compound of embodiment 82 having the formula:

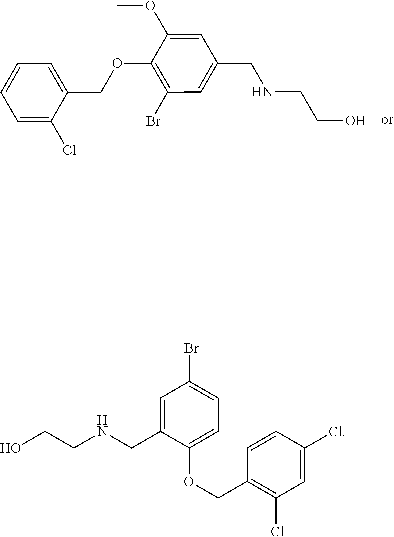 [SMILES: COc1cc(CNCCO)cc(Br)c1OCc1ccccc1Cl ; OCCNCc1cc(Br)ccc1OCc1ccc(Cl)cc1Cl]

84. A compound having the formula:

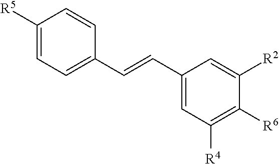

wherein, R², R⁴, and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O), —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁴ and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{7b}, R^{8b}, R_{9b}, and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, ml, v, and vl are independently an integer from 1 to 2; n and n1 are independently an integer from 0 to 4; X and X^{b} are independently —Cl, —Br, —I, or —F.

85. The compound of embodiment 84 having the formula:

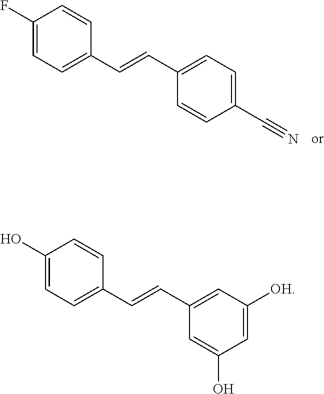 [SMILES: N#Cc1ccc(/C=C/c2ccc(F)cc2)cc1 ; Oc1ccc(/C=C/c2cc(O)cc(O)c2)cc1]

86. A compound having the formula:

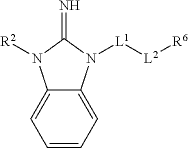

wherein, L¹ is a bond, substituted or unsubstituted alkylene, substituted or unsubstituted heteroalkylene, —NR^{1A}—, —O—, —S—, —C(O)—, or —CHR^{1A}—;
L² is a bond, substituted or unsubstituted alkylene, substituted or unsubstituted heteroalkylene, —NR^{1B}—, —O—, —S—, —C(O)—, or —CHR^{1B}—; R^{1A}, R^{1B}, R² and R⁶ are independently hydrogen, halogen, —CX₃, —CN, —SO₂Cl, —SOₙR¹⁰, —SOᵥNR⁷R⁸, —NHNH₂, ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

87. The compound of embodiment 86 having the formula:

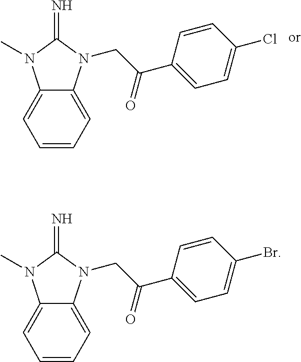 [SMILES: Cn1c(=N)n(CC(=O)c2ccc(Cl)cc2)c2ccccc21 ; "or" ; Cn1c(=N)n(CC(=O)c2ccc(Br)cc2)c2ccccc21]

88. A compound having the formula:

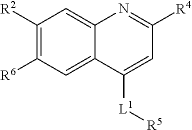

wherein, L¹ is a bond, substituted or unsubstituted alkylene, substituted or unsubstituted heteroalkylene, —NR^{1A}, —O—, —S—, —C(O)—, or —CHR^{1A}—, R^{1A}, R², R⁴, and R⁶ are independently hydrogen, halogen, —CX₃₅—CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R⁶ substituents may optionally be joined to form a substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃₅—CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted —SOᵥ₁NR^{7b}R^{8b}, heteroaryl; R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{7b}, R^{8b} and R^{10b} are independently hydrogen, halogen, —CF₃₅—CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, ml, v, and vl are independently an integer from 1 to 2; n and n1 are independently an integer from 0 to 4; X and X^{b} are independently —Cl, —Br, —I, or —F.

89. The compound of embodiment 88 having the formula:

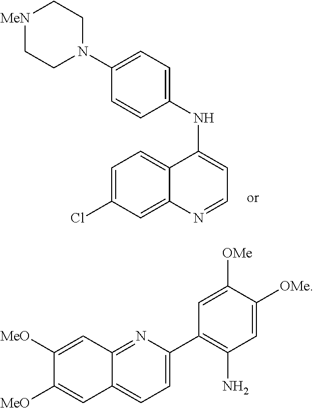

90. A compound having the formula:

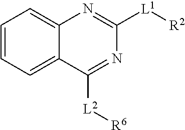

wherein, L¹ is a bond, substituted or unsubstituted alkylene, substituted or unsubstituted heteroalkylene, —NR^{1A}—, —O—, —S—, —C(O)—, or —CHR^{1A}—; L² is a bond, substituted or unsubstituted alkylene, substituted or unsubstituted heteroalkylene, —NR^{1B}—, —O—, —S—, —C(O)—, or —CHR^{1B}—; R^{1A}, R^{1B}, R² and R⁶ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

91. The compound of embodiment 90 having the formula:

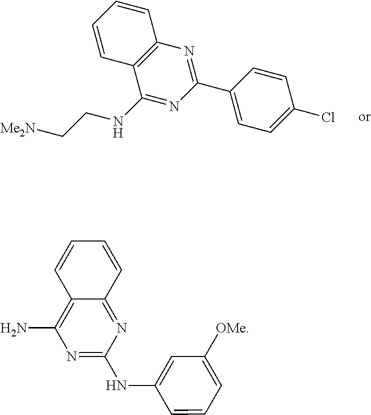 [SMILES: CN(C)CCNc1nc(-c2ccc(Cl)cc2)nc2ccccc12 ; COc1cccc(Nc2nc(N)c3ccccc3n2)c1]

92. A compound having the formula:

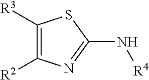

wherein, R², R³, and R⁴ are independently hydrogen, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m and v are independently an integer from 1 to 2; n is independently an integer from 0 to 4; X is independently —Cl, —Br, —I, or —F.

93. The compound of embodiment 92 having the formula:

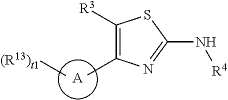

wherein, ring A is substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O)_{q}, —NR Jtc C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, p, q, and v are independently an integer from 1 to 2; n and r are independently an integer from 0 to 4; t1 is an integer from 0 to 8; X and X^{a} are independently —Cl, —Br, —I, or —F.

94. The compound of one of embodiments 92 to 93 having the formula:

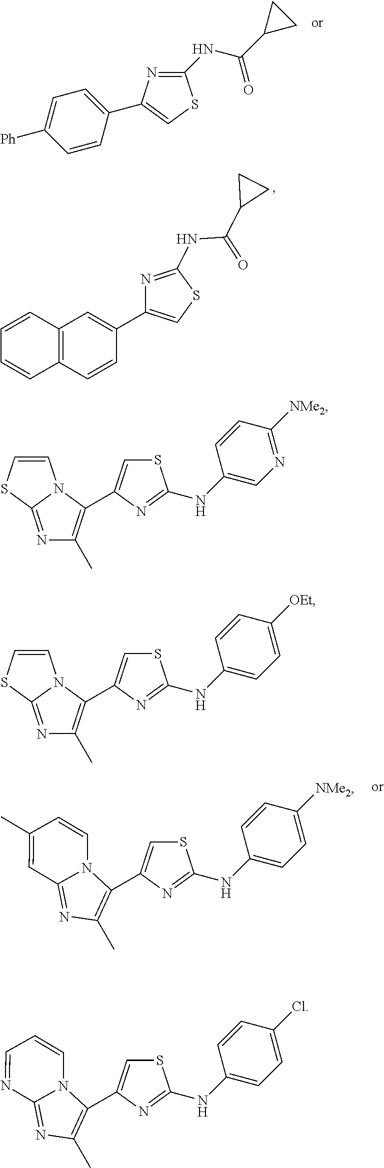

95. A compound having the formula:

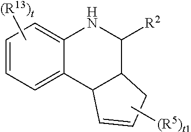

wherein, R² is independently hydrogen, halogen, —CX₃, —CN, —SO₂Cl, —SOᵥR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NeR^{8b}, —N(O)ₘ₁, —NR^{7b}R^{8b}, —C(O)R^{9b}, —C(O)—OR^{9b}, —C(O)NR^{7b}R^{8b}, —OR^{10b}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl; R^{7b}, R^{8b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is independently hydrogen, halogen, —CX^{a}₃, —CN, —SO₂Cl, —SOᵣR¹⁷, —SOₚNR¹⁴R¹⁵, —NHNH₂, —ONR¹⁴R¹⁵, —NHC═(O)NHNH₂, —NHC═(O)NR¹⁴R¹⁵, —N(O), —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl; R¹⁴, R¹⁵, R¹⁶, and R¹⁷ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; m, ml, p, q, v, and vl are independently an integer from 1 to 2; n, nl, and r are independently an integer from 0 to 4; t is an integer from 0 to 4; t1 is an integer from 0 to 6; X, X^{a}, and X^{b} are independently —Cl, —Br, —I, or —F.

96. The compound of embodiment 95 having the formula:

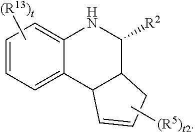

97. The compound of one of embodiments 95 to 96 having the formula:

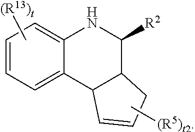

98. The compound of any one of embodiments 95 to 97 having the formula:

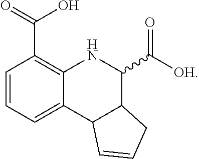 [SMILES: O=C(O)c1cccc2c1NC(C(=O)O)C1CC=CC21]

99. A pharmaceutical composition comprising a pharmaceutically acceptable excipient and a compound of any one of embodiments 45 to 98.

100. A method of treating a disease in a patient in need of such treatment, said method comprising administering a therapeutically effective amount of a compound of any one of embodiments 45 to 98.

101. The method of embodiment 100, wherein the disease is a neurodegenerative disease.

102. The method of embodiment 101, wherein the disease is a prion disease.

103. The method of embodiment 102, wherein the disease is Creutzfeldt-Jakob disease.

104. The method of embodiment 101, wherein the disease is Alzheimer's disease, Amyotrophic lateral sclerosis, Huntington's disease, or Parkinson's disease.

105. The method of embodiment 104, wherein the disease is Alzheimer's disease.

106. A method of decreasing the amount of a prion protein in a cell, said method comprising contacting said cell with a compound of any one of embodiments 45 to 98.

107. A method of decreasing the amount of amyloid beta in a patient, said method comprising administering to said patient an effective amount of a compound selected from the group consisting of the compounds in embodiments 1 to 36 and 45 to 98.

108. A method of decreasing the level of activity of gamma secretase in a patient, said method comprising administering to said patient an effective amount of a compound selected from the group consisting of the compounds in embodiments 1 to 36 and 45 to 98.

### VI. Examples

### A. Screening Overview

Using novel, reliable and robust HTS assays and methods, we discovered diverse chemical leads and selected analogs that have good potency in lowering levels of PrP^{C} in human neuroblastoma and glioblastoma cell lines as well as those lowering levels of PrP^{Sc} in dividing cells and nondividing ScN2a-c13 cells.

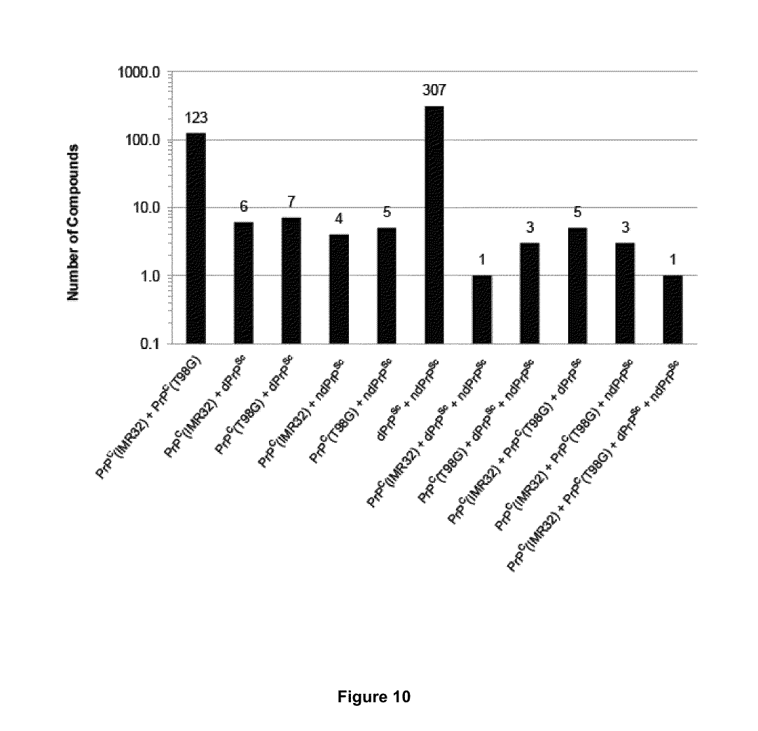

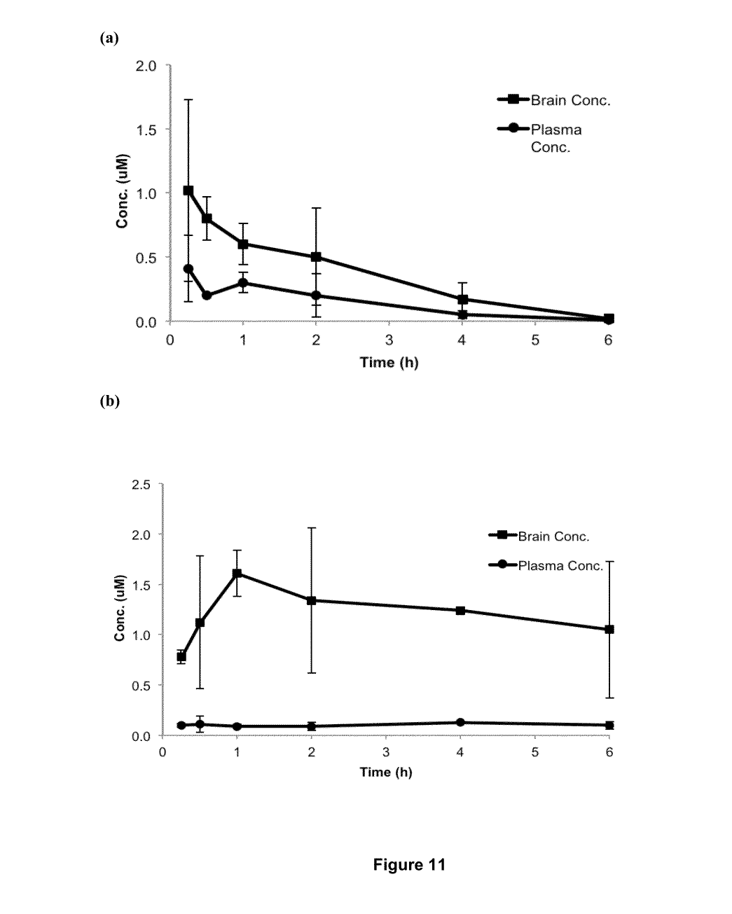

From 123 confirmed SPC hits in the PrP^{C} assays (FIG. 10) and 307 confirmed SPC hits in the dividing and nondividing PrP^{Sc} assays, 13 compounds were found to reduce both PrP^{C} and PrP^{Sc} levels. One of these, a tetrahydroquinoline, was active in all assays. All of these compounds and the leads they represent were nontoxic to cells, as measured by the calcein AM assay. It is not surprising the PrP^{C} and PrP^{Sc} assays yielded different SPC hits. This may be due to the fact that different cell lines and different assay conditions were used in each. Several purchased analogs from hits that reduced PrP^{Sc} in cells also attained high brain concentrations in mice after oral dosing, including the two shown (FIG. 11b). Optimization of these analogs for “drug-like properties” is underway.

We also tested FDA-approved drugs in the PrP^{C} and PrP^{Sc} assays. From the HTS of 1,700 FDA-approved drugs, we found four that lowered levels of both PrP^{C} and PrP^{Sc}, including three HMG-CoA reductase inhibitors (simvastatin, fluvastatin and pravastatin) and the antiprotozoal drug, quinacrine. The other 15 FDA drugs positive in the PrP^{Sc} assay (19 total) were similar to those previously reported by others. Additionally, 50 FDA-approved drugs were positive in the PrP^{C} ELISA and EC₅₀ assays and potency estimates were generated for both the ELISA and the calcein cell viability assays. However, because the EC₅₀ concentrations needed for antiprion efficacy for both PrP^{Sc} and PrP^{C} ELISA assays typically ranged from 1-10 μM and they were within 2- to 5-fold those causing cell viability issues in the calcein assays, the therapeutic index for these may be small.

With one exception, none of the drugs or experimental compounds previously identified to lower PrP^{Sc} levels in murine cell lines has increased the survival of prion-infected mice. “Compound B,” which extended the incubation times in prion-infected mice (Kawasaki, Y. et al., J. Virol. 2007, 81, 12889), may not be acceptable for use in humans because it contains a hydrazone moiety that is metabolically unstable and could likely lead to a reactive intermediate causing potentially serious adverse effects, drug-drug interactions, or both (Jonen, H. G. et al., J. Biol. Chem. 1982, 257, 4404; Malca-Mor, L.; Stark, A. A. Appl. Environ. Microbiol. 1982, 44, 801; Walton, K. et al., Carcinogenesis 1997, 18, 1603). Indeed, our studies with Compound B revealed lethal toxicity when administered in vivo at doses ≧150 mg/kg/day.

Quinacrine, an FDA-approved drug reported to lower levels of PrP^{Sc} in prion-infected cells, was surprisingly shown to be ineffective in prion-infected mouse models (Collins, S. J. et al., Ann. Neurol. 2002, 52, 503; Gayrard, V. et al., J. Pharmacol. 2005, 144, 386; Nakajima, M. et al., Dement. Geriatr. Cogn. Disord. 2004, 17, 158). A recent report found that quinacrine was ineffective over time in nondividing cells, which led to the postulation that drug-resistant prions were forming in culture over the course of the 5-day assay (Ghaemmaghami, S. et al., PLoS Pathog. 2009, 5, e1000673). This possibility might explain the failure of quinacrine to extend survival of prion-infected mice and argues that antiprion compounds may need to be potent in both dividing and nondividing prion-infected cells in order to be effective in vivo in mice and patients with prion disease. Despite its potency in dividing cells and successive extension of survival in infected mice, Compound B is inactive in nondividing cells. We identified experimental compounds that are active in dividing and nondividing cells, and will test these candidates in prion-infected mouse models.

Our results show that with reliable HTS assays in hand to identify compounds that lower both PrP^{C} and PrP^{Sc} levels in dividing and nondividing cells, it has been possible to generate hits and novel leads. In our search for hits, we used predictions of bioactivity and a metric of dissimilarity (Keiser, M. J. et al., Nat. Biotechnol. 2007, 25, 197), (Jonen, H. G. et al., J. Biol. Chem. 1982, 257, 4404) to prioritize our screening libraries where the top 650 of 3,014 plates were selected based on their SEA scores. This strategy successfully identified and confirmed many leads for each of the four assays. Furthermore, SAR-by-catalog successfully identified many analogs that led to the identification of several promising new leads: two compounds have shown to be absorbed orally and reach the brain at high concentrations. With many scaffolds now identified from each of the assays, along with many purchased analogs tested and found to be potent, a major effort is now underway to use SAR, driven by medicinal chemistry, pharmacokinetics, formulations, and toxicology from each series and assays. The candidate compounds will be evaluated in prion-infected animals. SAR-by-synthesis programs are now underway to optimize these promising leads to ensure they have good overall drug-like properties. Together, these strategies and efforts offer promising progress towards developing a cocktail of effective therapies for human prion diseases.

### B. Aminothiazole SAR Overview

The antiprion action of new aminothiazole analogs was evaluated using a new ‘clone-3’ cell line (denoted ScN2a-c13(Ghaemmaghami, S et al., J Biol Chem 2010, 285, 10415-10423)) that expresses a higher level of PrP^{Sc} as compared to the ScN2a cell line. In general we have found that EC₅₀ values for antiprion compounds tend to be 10-fold higher in the high-expressing cells, and thus the ScN2a-c13 cell line represents a more stringent test of antiprion action. The EC₅₀ values presented in the discussion below represent mean values from three separate determinations using ScN2a-c13 cells. The precision of the assay is high; the coefficient of variance in mean pEC₅₀ values is generally less than 5%. The high quality of the assay data allowed even subtle SAR trends to be assigned with some confidence. An evaluation of compound toxicity toward ScN2a-c13 cells was carried out using the fluorescent probe calcein-AM (Thompson, M. J. et al., J Med Chem 2009, 52, 7503-7511). Almost without exception we found that 2-aminothiazole analogs are not toxic to ScN2a-c13 cells, indicating that 2-aminothiazoles reduce PrP^{Sc} load in ScN2a-c13 cells by a drug-like mechanism (i.e., not simply by killing cells). Notably, 2-aminothiazoles apparently do not reduce PrP^{Sc} load in non-dividing ScN2a-c13 cells that have been arrested in cell division by treatment with sodium butyrate. It should be noted however that lack of activity in non-dividing cells does not necessarily preclude antiprion efficacy in animals, as we found that hydrazone 2, like 2-aminothiazoles, has no effect on non-dividing ScN2a-c13 cells.

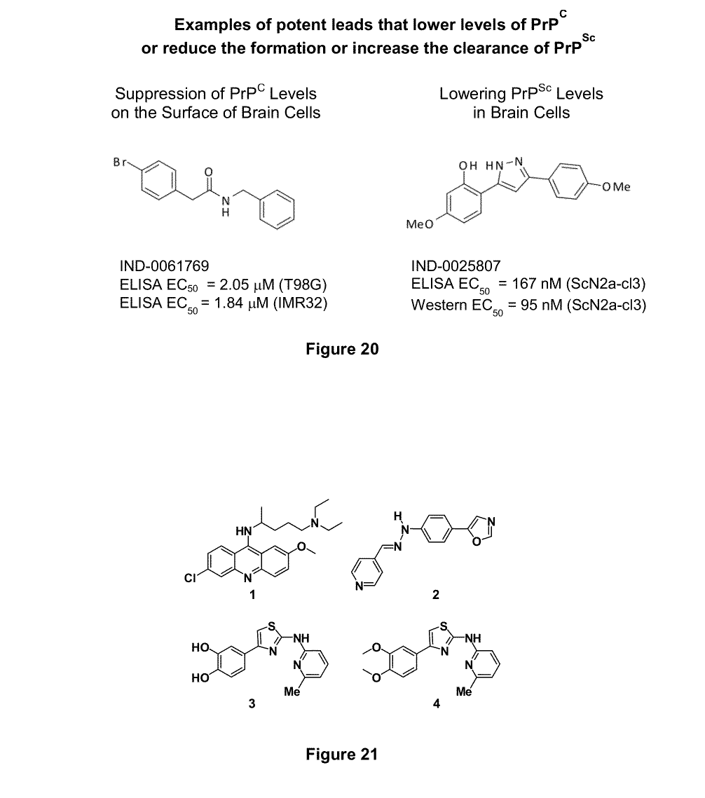

The SAR studies described herein were undertaken with the dual objectives of expanding upon nascent SAR (Ghaemmaghami, S. et al., J Virol 2010, 84, 3408-3412) and identifying improved aminothiazole analogs with a higher likelihood of penetrating the brain in animals. To help achieve the latter objective, we applied recently advanced (Hitchcock, S. A. et al., J Med Chem 2006, 49, 7559-7583) guidelines for assessing the potential CNS activity of small molecules. These “rules of thumb” advise special attention to properties such as molecular weight (<500 Da. preferred), polar surface area (<90 Å² preferred), clogP (2-5 preferred), and the number of hydrogen bond donors (<3 preferred). The synthesis of new 2-aminothiazole analogs was carried out in both serial and parallel formats using the Hantzsch-type condensation of bromomethyl ketones with thioureas (Scheme 2). An early objective of the SAR study was to modify the catechol ring present in early screening hits like 3, since such functionality would likely limit brain exposure in vivo, and might also present metabolic and/or toxicological liabilities. Evaluation of the corresponding dimethoxyphenyl analog 4 (FIG. 21) showed it to be equipotent to 3, thereby alleviating concerns that a catechol A-ring might be required for antiprion activity. With this potential liability eliminated, a systematic exploration of aminothiazole SAR was initiated.

Preliminary SAR (Ghaemmaghami, S. et al., J Virol 2010, 84, 3408-3412) suggested a preference for 2-pyridyl type C-rings over simple aryl congeners. To evaluate more fully the C-ring SAR, a series of analogs were synthesized with alkyl, aryl, or heteroaryl groups at this position (Chart 1). The N-alkyl analog (5) was without significant activity while among regioisomeric pyridyl analogs, 2-pyridyl analog 7 was indeed more potent than the 3-pyridyl or 4-pyridyl congeners (8 and 9). Hence, analog 7 had an EC₅₀ value (defined as the effective concentration for reducing PrP^{Sc} load in ScN2a-c13 cells by 50%) of 1.22 μM, roughly ten-fold lower than the original (Ghaemmaghami, S. et al., J Virol 2010, 84, 3408-3412) screening hits. Replacement of the 2-pyridyl ring in 7 with 2-pyrimidyl or 2-pyrazinyl rings produced analogs of comparable (10) and reduced (11) potency, respectively. Next, we examined ring-substitution effects in the favored 2-pyridyl C-ring type. In general, ring substitution was most favorable in positions distal from the B-C ring connection, as in methyl substituted analogs 14 and 16, and especially in the more extended bicyclic C-ring analogs 17-19 (EC₅₀=0.11 μM for 17). In contrast, analogs substituted proximally to the B-C ring connection (e.g., 15, 20) were notably less potent than their unsubstituted comparators. Electronic effects in the C-ring appear to be of comparably smaller importance; analogs with electron rich (13) or electron deficient (12) 2-pyridyl rings showed similar activities. The most potent analogs identified from this series were those with more extended bicyclic C-ring systems. Hence, quinoline, isoquinoline, and naphthalene analogs (17-19) were approximately ten-fold more potent than comparable monocyclic analogs, and at least 100-fold more potent than the original screening hits.

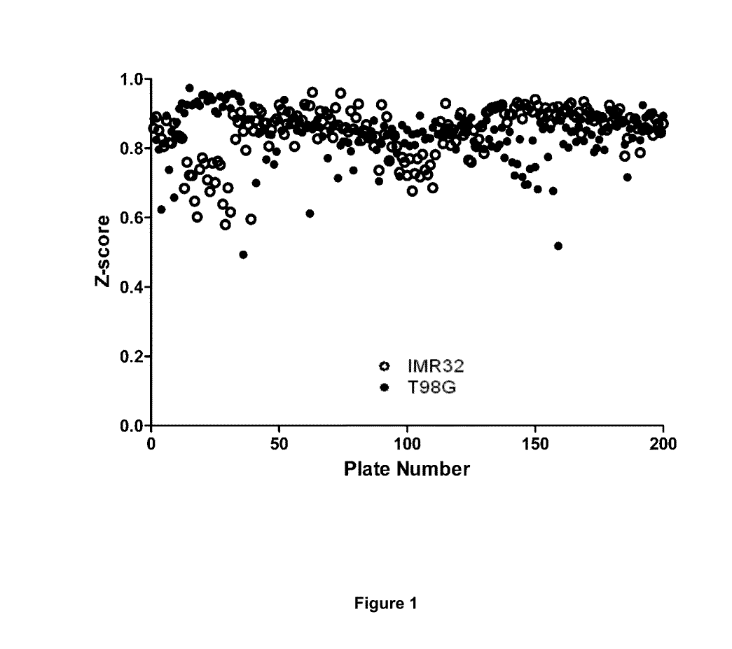

Having identified several viable new C-ring subtypes, we next explored SAR of the ‘A-ring’ positioned at C-4 of the aminothiazole ring (FIG. 2). As noted above, bis-methylation of the catechol function in 3 to afford 4 was a tolerated modification. A more systematic exploration of A-ring preferences was carried out in the context of the favored 2-pyridyl and isoquinoline C-ring types. Various aromatic and heteroaromatic ring systems could be tolerated (Chart 2), but analogs tested bearing small alkyl groups at this position were either inactive (29, 36) or less potent (37). Analogs 25 and 33 bearing unsubstituted phenyl A-rings were between two and ten-fold less potent than pyridyl (23, 24, 32) or para-methoxyphenyl A-ring analogs (21, 30). Whereas para-methoxyphenyl analog 30 was among the most potent analogs examined (EC₅₀=0.34 μM), the analogous trifluoromethoxyphenyl analog 31 was surprisingly inactive in these experiments. Other favorable A-rings conferring low or sub-micromolar potencies included phenyl-substituted isoxazoles as found in analogs 27 and 35, and pyridyl-substituted thiophenes as in analogs 26 and 34.

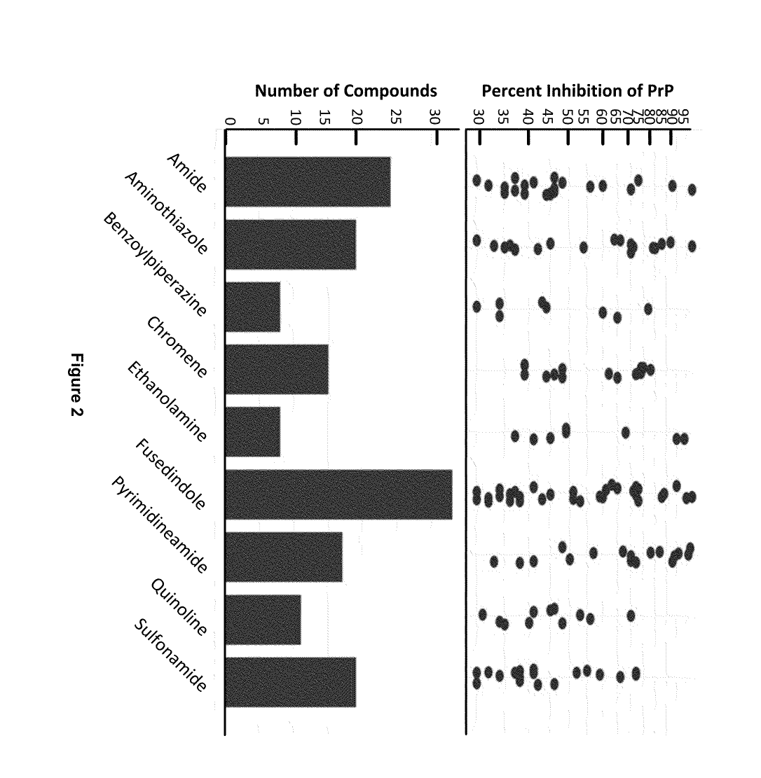

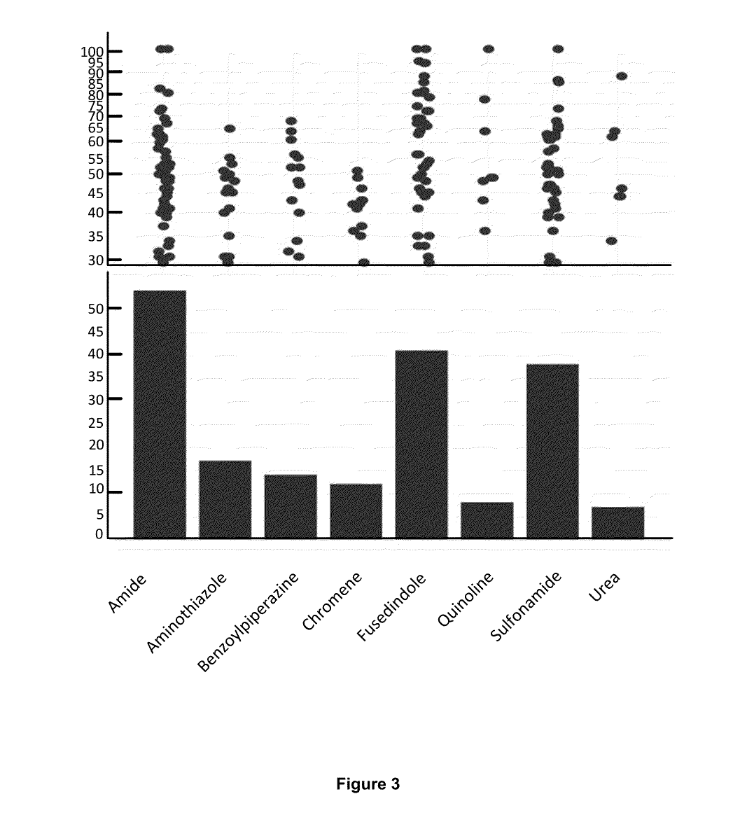

The general tolerance of para or meta substitution on the A-ring can be contrasted with an apparent intolerance for substitution at the ortho position in these experiments. This effect was evident in both six-membered (compare 21 and 22) and five-membered (compare 27 and 28) A-rings and was true regardless of C-ring chemotype. These findings suggest perhaps that a co-planar arrangement of the A- and B-ring is important for activity, the presence of an ortho substituent as in 22 and 28 disfavoring such a conformation. To test this supposition, we prepared fused tricyclic analogs 38 and 39 (FIG. 3) in which a co-planar conformation is enforced by A-B ring fusion. In both cases the A-B ring fusion was tolerated, being somewhat favored in the case of 38 (as compared to 25) and somewhat disfavored in the case of 39 (as compared to 14). This observation that ortho substitution is tolerated only in the context of A-B ring fusion in these experiments supports the notion that a co-planar conformation of these ring systems may be important for activity.

Among the new A-ring variants examined (Chart 2), phenylisoxazole 27 was notable for its sub-micromolar potency and improved stability to rat liver microsomes as compared to phenyl (25) and pyridyl (23) congeners (Table 21). This finding led to a reinvestigation of favored C-ring types in the context of the phenylisoxazole A-ring (Chart 3). Perhaps not surprisingly, the SAR of phenylisoxazole A-ring analogs was not completely reconcilable with SAR in the original dimethoxyphenyl A-ring series. Thus, whereas extended bicyclic C-rings (isoquinoline, quinoline) were optimal in combination with the dimethoxyphenyl A-ring (Chart 1) in these experiments, the most potent phenylisoxazole analogs were those bearing methoxypyridine C-rings in these expimeriments, as in analogs 40 (EC₅₀=0.23 μM) and 41 (EC₅₀=0.25 μM). By comparison, other pyridine (27, 42-45) and quinoline (35) C-ring analogs were between four- and twenty-fold less potent.

We also examined SAR relating to the nature of connection between the A-, B-, and C-rings. For example, insertion of an amide function between the A- and B-ring in analog 14 produced analog 46 of comparable potency (Chart 5). However, the consequent introduction of an additional hydrogen bond donor in 46 was judged undesirable in these experiments with respect to CNS properties and so amide-linked analogs like 46 were not pursued further. With respect to the B-C ring connection, methylation (as in 49) or acylation (as in 50) of the amine linkage in analog 17 was well tolerated. In contrast, replacement of the amine linkage in 17 with amide linkages (as in 47 and 48) led to a ˜100-fold loss of potency in these experiments (Chart 5). Overall, activity data derived from analogs 47-50 suggest that proper spacing of the B- and C-rings is important for antiprion activity, whereas the presence of a hydrogen bond donor in the B-C ring linkage is not. This latter finding is significant since the complete elimination of hydrogen bond donors in analogs like 49 and 50 would predict for better permeability across the BBB in animals.

The SAR studies described above have revealed a number of structural determinants in the antiprion activities of 2-aminothiazoles. Just as importantly, many of the new analogs possess physiochemical properties that predict permeability across the BBB. In fact, most small molecules do not readily traverse the BBB and/or are subject to active efflux mediated by drug resistance transporters (e.g., P-glycoprotein transporter; P-gp) expressed in the endothelial cells that constitute the BBB (Aller, S. G. et al., Science 2009, 323, 1718-1722). Efflux by P-gp is much more difficult to predict than is passive permeation of the BBB. To address the potential for efflux, a subset of aminothiazole analogs were evaluated for permeability in P-gp-expressing Multidrug resistance-1 Madin-Darby canine kidney (MDR¹-MDCK) cell mono-layers, an assay that has been utilized as an in-vitro predictor of in-vivo BBB permeability (Braun, A. et al., Eur J Pharm Sci 2000, 11 Suppl 2, S51-S60). All eight analogs evaluated showed good permeability in this assay, and more importantly, none appeared likely to be P-gp substrates based on absorptive (apical to basalateral) and secretory (basalateral to apical) permeability values (Table 21). In fact, analogs 28, 12, 17, and 18 showed greater permeability in the absorptive direction, indicating net active transport across the BBB. Furthermore, analogs 17 and 18, as well as 12 and 27, displayed excellent stability to rat liver microsomes in vitro. On the basis of antiprion potency, metabolic stability, and permeability, optimized 2-aminothiazole analogs like 27, 13, 17, and 18 were considered as candidates for further study in animals.

Presented herein are results of a representative feeding experiment in which compound 27 was administered at escalating doses (0, 40, 80, 130, or 210 mg/kg/day) to wild-type FVB mice for three days as part of a rodent liquid diet (FIG. 23). This protocol is suitable for subsequent animal efficacy trials, where daily dosing for well over 100 days is required (administration by oral gavage is not practical for such long-term experiments). Brain and plasma concentrations of compound 27 were measured after the 3-day administration period. Increasing doses of 27 resulted in a linear increase in plasma concentrations (FIG. 23). Doses up to 130 mg/kg/day also resulted in linear increases in brain concentrations. While significant variability between animals was seen at the two highest doses, concentrations of 27 in brain generally exceeded those in plasma. Mean brain concentrations of 27 were in excess of the compound's in vitro activity (EC₅₀=0.94 μM), surpassing it by as much as 25-fold at the higher doses. Since the reported concentrations were determined at an arbitrary time point following three days of feeding, they represent pseudo steady-state rather than peak concentrations. Full pharmacokinetic parameters were not determined as part of this study, as the intent was to evaluate drug concentrations in brain and plasma at pseudo steady-state. Differences in feeding behavior among individual animals may partially explain the observed variability within certain animal cohorts. Overall, the excellent brain concentrations achieved in these studies confirm that 2-aminothiazole analogs such as 27 are absorbed following oral administration and achieve and maintain high concentrations in the brains of animals. These results prompted us to select several 2-aminothiazole analogs as candidates for further investigation in mouse models of prion disease.

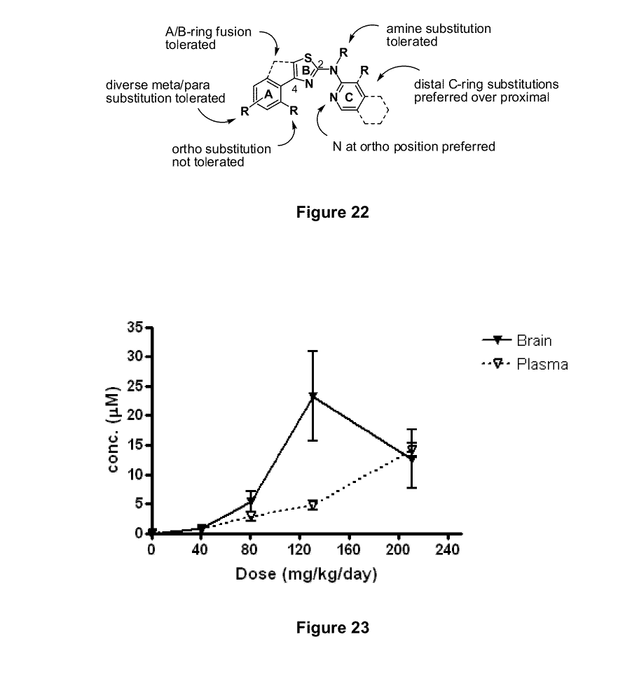

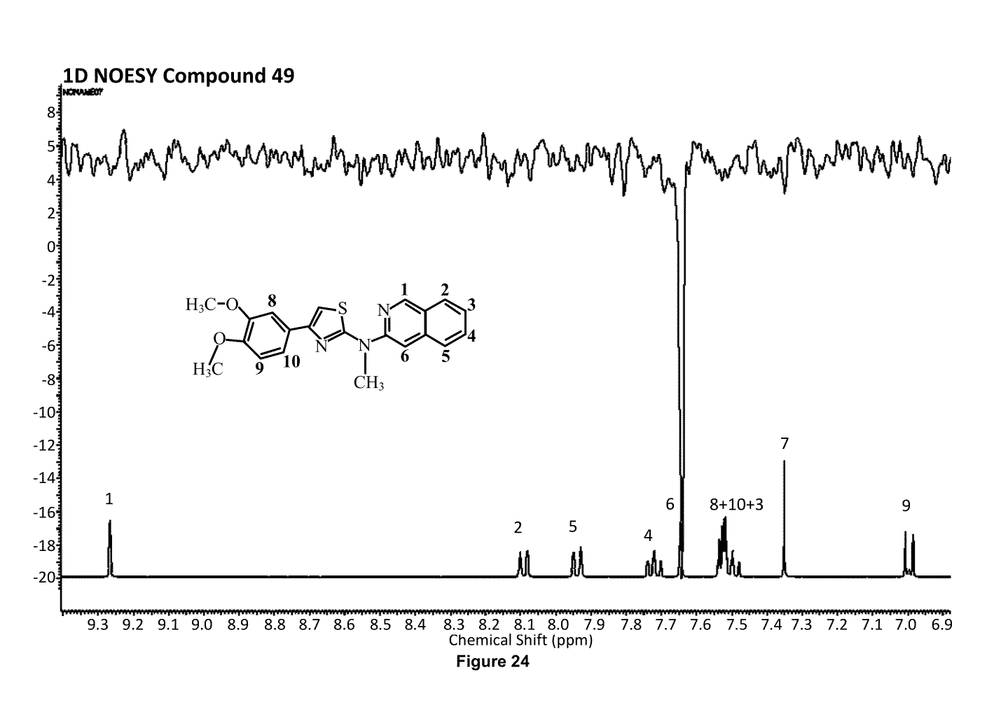

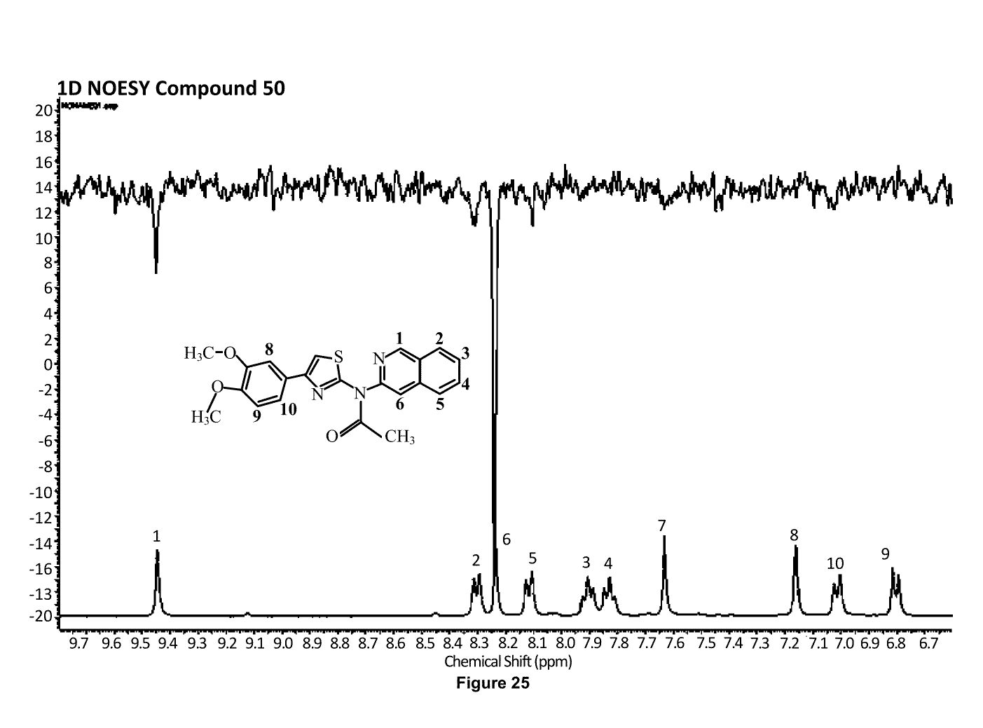

Preliminary mechanistic profiling of aminothiazole analogs indicated that they neither diminish the expression of PrP^{C} nor denature PrP^{Sc}, thus suggesting that a mechanism influencing PrP^{Sc} formation or clearance is more likely (Ghaemmaghami, S. et al., J Virol 2010, 84, 3408-3412). For example, the compounds might inhibit as-yet unidentified auxiliary macromolecules (Perrier, V. et al., Proc Natl Acad Sci USA 2000, 97, 6073-6078) that promote prion replication or enhance the activity of proteins that facilitate the clearance of PrP^{Sc}. The SAR studies reveal those positions in 2-aminothiazole structure that are sensitive or insensitive to modification (FIG. 22).

### C. Overview of IND24 and IND81 Characterization

We have identified improved 2-aminothiazole analogs that possess EC₅₀ values as low as 81 nM in ScN2a-c13 cells. The SAR revealed in this study suggests action at one or more defined molecular targets, the identification of which remains to be established. The physiochemical properties of many 2-aminothiazole analogs are favorable for possible therapeutic use in prion diseases. Preliminary animal studies demonstrate that members of the 2-aminothiazole class are orally absorbed when formulated appropriately in liquid rodent diet and can achieve steady-state brain concentrations well in excess of their in vitro potencies.

Here we report two 2-AMT analogs, IND24 and IND81, selected for preclinical development (from among 235 2-AMT analogs synthesized). Both IND24 and IND81 a) had oral bioavailability of 27-40% in mice; b) had major metabolites involve ring hydroxylation; c) were not substrates for the Mdrl efflux pump transporter (P-glycoprotein); d) achieved brain concentrations >10× their respective EC₅₀ values in cells; and e) had free fractions in plasma and brain of 6-9% in mice. IND81 was metabolized potentially by several major human cytochrome (P450) isoforms, Compound B (hereafter referred to as IND54304) (Kawasaki, Y. et al., J Virol 2007, 81, 12889-12898 #8036; Teruya, K. et al., Infect. Disord. Drug Targets 2009, 9, 15 #9004) was also pharmacokinetically profiled to determine doses as a positive control in future efficacy studies to evaluate IND24 and IND81.

There are no treatments or cures for prion diseases, including CJD, which is a rapidly progressive and fatal neurodegenerative disease. To date, 235 analogs in the 2-AMT series have been synthesized and evaluated for antiprion potency (EC₅₀) using a new ELISA assay for PrP^{Sc}. EC₅₀ values determined by ELISA agreed with those found by Western immunoblotting.

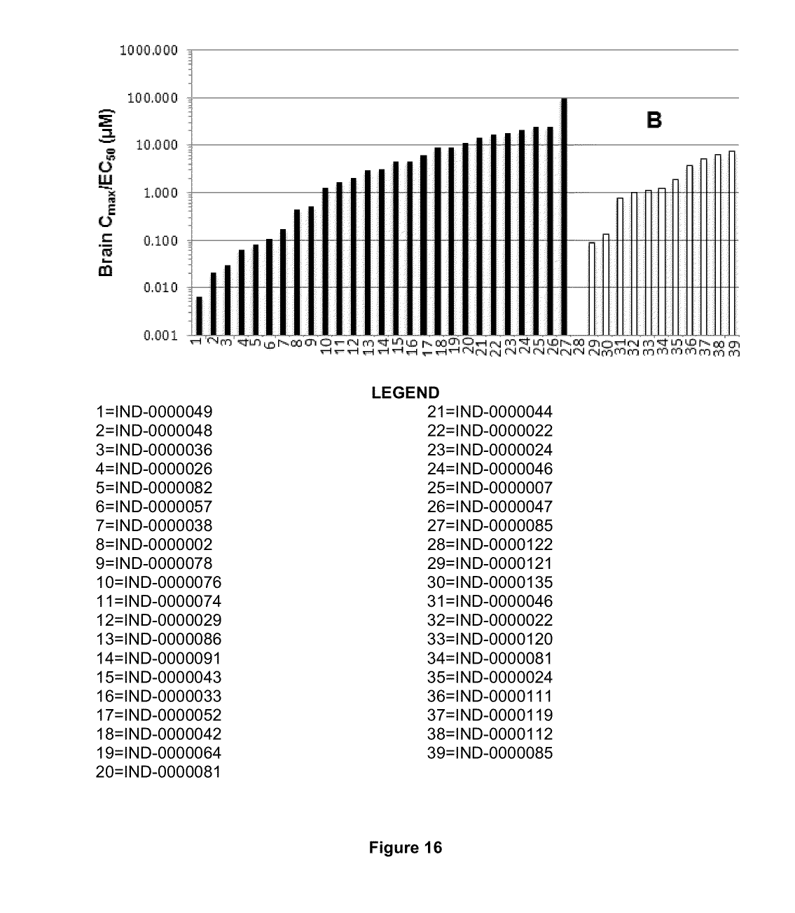

In the present work, we evaluated a subset of the 235 analogs to select at least two compounds for preclinical testing. We aimed to identify leads suitable for proof-of-concept testing in prion-infected mouse models {Korth, 2003}; {Giles, 2010}. Thus far, no drug has been able to extend survival in these models beyond ˜70-150 days, depending on the model. From the first ˜100 analogs made, we evaluated 34 in single-dose pharmacokinetic studies to determine if any would be good preclinical development candidates (Tables 8 and 9). Selection was based on assessing drug-like properties, which included low EC₅₀ values (Tables 8 and 9), good solubility (Table 9), good oral bioavailability, good predicted potential for brain delivery (efflux ratios, Table 10) and the ability to achieve brain concentrations exceeding the EC₅₀ value by ≧10× (FIG. 16). From these, we selected 10 for multiple-dose pharmacokinetic studies focusing on “steady-state” concentrations (Cₛₛ) in brain homogenate, AUC in brain homogenate, and the ability to maintain Cₛₛ values ≧10× the EC₅₀ value over time. Several promising compounds were identified, including IND24 and IND81.

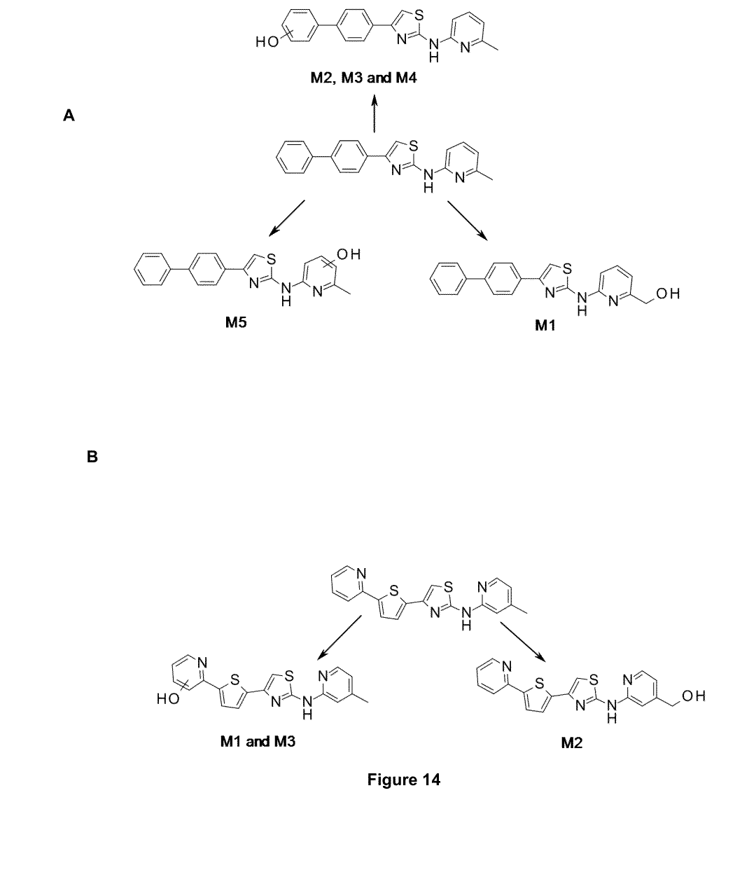

In general, all 34 compounds had lower aqueous solubility at pH 2 or 4, but had higher solubility in FaSSIF and in cell media, which was used as an indirect measure of solubility in the presence of proteins (Table 9). Permeability studies were then performed for 10 selected compounds to get an initial assessment of any potential issues that would suggest that some might be substrates for P-gp. The efflux ratios (Table 10) suggested that none were substrates of P-gp. Metabolic stability was performed on the same 10 selected compounds to evaluate the t_{1/2} in mouse, rat and human microsomes, along with corresponding hepatic extraction ratios. As can be seen in Tables 4 and 5, there was a wide range of stability in human, mice and rat microsomes between compounds. Importantly, metabolic results suggested that IND24 should have excellent stability in humans and mice. While hydroxylated metabolites of IND24 could be identified in human liver microsomes (FIG. 14), the role of any specific P450 isozyme could not be ascertained. Similarly, several hydroxylated metabolites could be identified for IND81 (FIG. 14) with CYP1A2, CYP2D6, CYP3A4, and CYP2C19 to a smaller extent implicated in its metabolism. Binding of IND24 and IND81 was evaluated in plasma and 20% brain homogenate at 1 and 10 μM. While the free fraction in plasma differed somewhat between mouse, rat, dog, and human, they were all in the same general range of 57%. The nonbound free fraction in brain ranged from 7-8% for IND24 and was ˜9% for IND81 (Table 13).

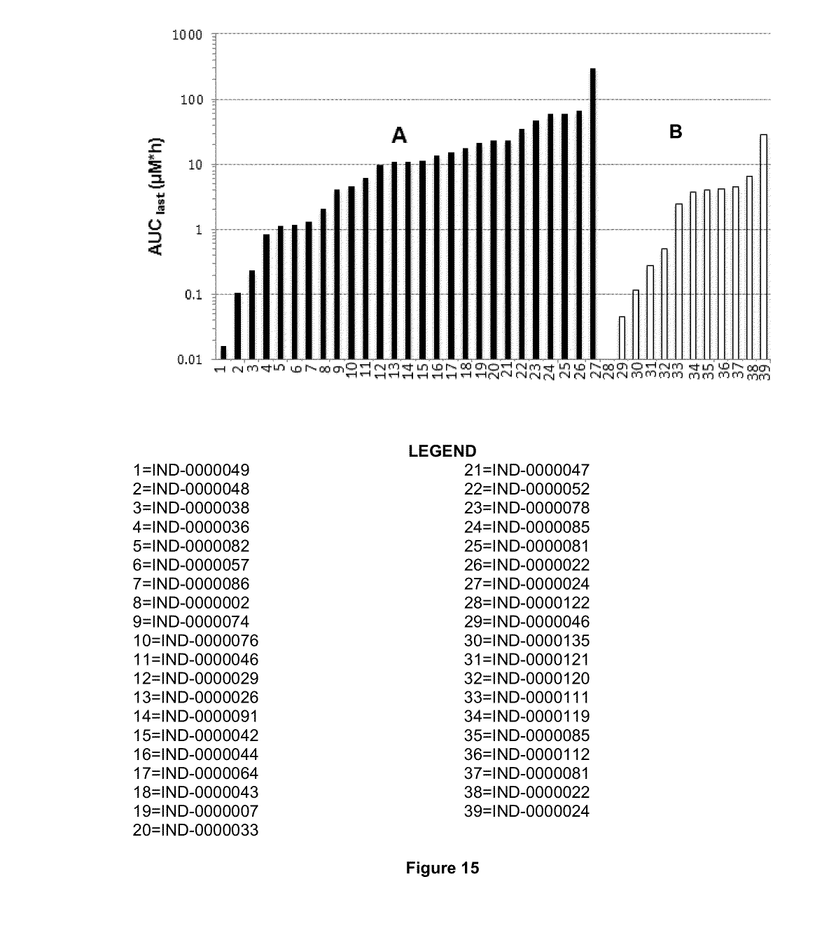

Pharmacokinetic studies were performed for IND24, IND81, and IND54304 following IV (1 mg/kg) and oral (10 mg/kg) doses. The results show that the absolute bioavailability of the two AMT and Compound B ranged from 27-40% in mice, at least under the conditions of the study, which included the use of an excipient. The t_{1/2} was longer for the AMT compounds, especially IND24, following IV dosing (Table 16). AMT were studied in single dose oral pharmacokinetic studies at 40 or 10 mg/kg. Initially, 27 were dosed at 40 mg/kg in order to ensure that measurable concentrations would be above the lower level of quantitation by LC/MS/MS. Ten (including three that were repeated) were studied at 10 mg/kg. AUC_{0->last} ranged from 0.02-500 μM and <0.01-40 μM*h after the 40 and 10 mg/kg doses, respectively (FIG. 15).

IND24 and IND81 showed the highest AUC values in brain of the compounds tested. In addition, the ratio of brain concentrations to EC₅₀ values obtained in neuronal cells after single oral doses of 40 or 10 mg/kg, ranged from 0.008-100 and <0.01-8, respectively (FIG. 16). Because IND24 and IND81 were among the best after the 40 and 10 mg/kg doses, and have good overall drug-like properties, they will be selected to advance to in-vivo animal studies to evaluate their potential to extend survival in the prion-infected models. IND85, an analog most recently identified, actually had a better Cₘₐₓ:EC₅₀ ratio after either dose and will be further evaluated as another potential preclinical development candidate.

Experiments were performed to evaluate pharmacokinetics, including brain delivery, for the two optimized leads over a wide range of doses that could be used for 300 days, or longer. This would require the use of a liquid formulation and diet in order to minimize animal handling during a 300+ day study. To achieve good oral bioavailability and good target drug concentrations in the brain, we showed that it was helpful to add PEG400 to the oral formulation of the drug, which was added to the liquid diet. This enhanced drug dissolution (pH dependent solubility), absorption and oral bioavailability.

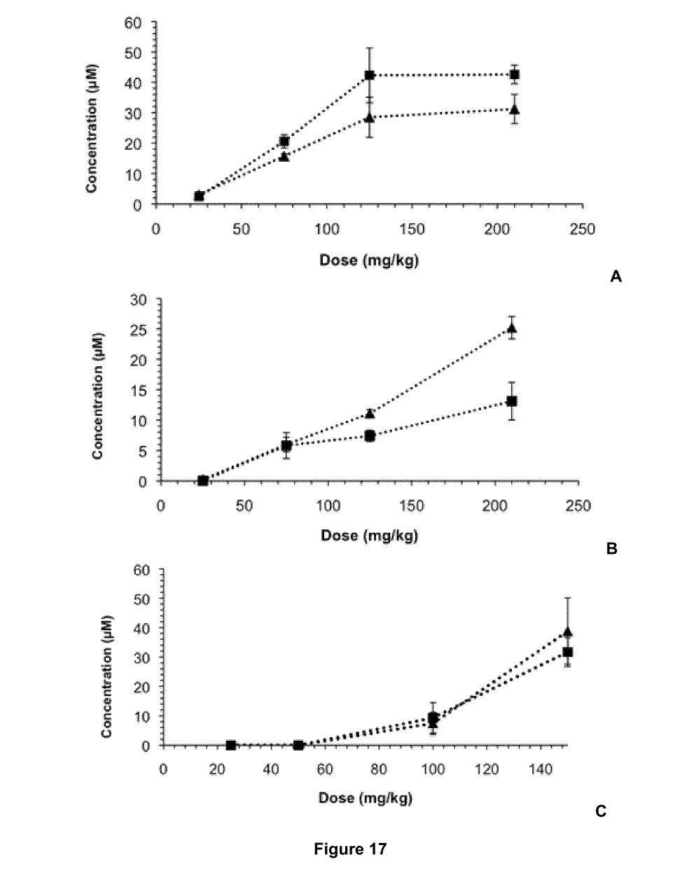

Some important pharmacokinetic studies were those involving three-day dosing to yield Cₛₛ concentrations in brain and plasma. Cₛₛ values well below the EC₅₀ should lead to sub-therapeutic drug concentrations in prion-infected mouse models, while concentrations >10×EC₅₀ should support a proof-of-concept experiment in the planned prion-infected mouse experiments. For both IND24 and IND81, brain concentrations were above 10×EC₅₀ at doses between 50-125 mg/kg/day, where the dose of PEG400 was 0.125% (v/v). Linearity in brain and plasma concentrations was better for IND81 (FIG. 17). Interestingly, concentrations did not increase linearly in brain or plasma after IND54304, suggesting the potential for dose-dependent or Michaelis-Menten kinetics.

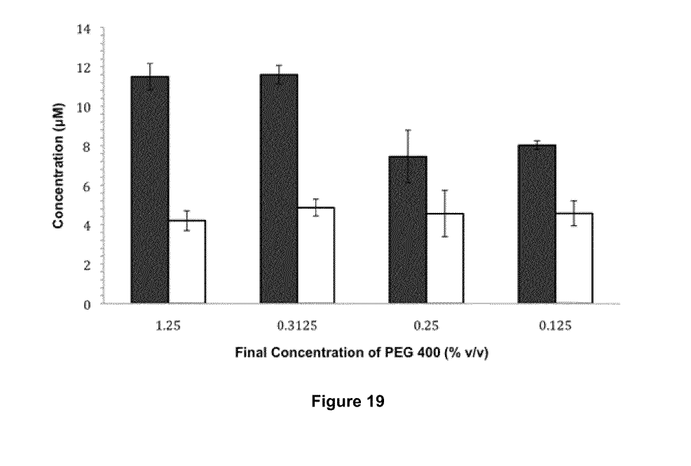

We evaluated a range of PEG400 dose in the diet, seeking to maximize exposure of drug in brain while minimizing the daily load of PEG400. We showed that PEG400 doses as low as 0.125% resulted in similar drug exposure as doses of 1.25% (FIG. 19). We evaluated the linearity in brain and plasma drug exposure for both drugs over a dose range of 1-210 mg/kg/day. We wanted to identify doses that would lead to “steady-state” drug concentrations in the brain that ranged from <EC₅₀ in cells to >10×EC₅₀. We wanted to determine pharmacokinetic-pharmacodynamic relationships in the planned studies in prion-infected mouse models that would evaluate effects on survival and preclinical safety margins. The drug in diet approach is ideal since it simplifies drug administration, achieving and maintaining drug concentrations for long periods each day as mice are nocturnal and feed/drink for approximately 12 h/day. It was also crucial to have a dosing regimen that could be tolerated for up to 300 days or longer without the need to frequently handle mice daily over long periods of drug treatment. We showed that target brain concentrations (10×EC₅₀ values in cells) could be achieved and maintained with doses as low as 50 mg/kg/day with the addition of 0.125% PEG400 in the diet. A range of doses expected to be ineffective as well as effective in extending survival in prion-infected mice have been started to define the doses, exposure, and dose-response projected to ultimately achieve efficacy in patients with CJD.

It was also important to characterize “Compound B” in pharmacokinetic studies to determine doses and formulations to be used as a positive control in upcoming efficacy studies in prion-infected mice. As discussed previously, this compound extended the incubation times in prion-infected mice (Kawasaki, Y. et al., J. Virol. 2007, 81, 12889), but may not be acceptable for use in humans because it contains a hydrazone moiety that is metabolically unstable and would likely lead to a reactive intermediate causing potentially serious adverse effects, drug-drug interactions, or both (Jonen, H. G. et al., J. Biol. Chem. 1982, 257, 4404; Malca-Mor, L.; Stark, A. A. Appl. Environ. Microbiol. 1982, 44, 801). Indeed, our own studies with Compound B (referred to as IND54304) revealed lethal toxicity when administered chronically in vivo for eight days at doses >110 mg/kg/day. We were able to show that doses up to 100 mg/kg/day could be tolerated for at least eight days and resulted in C concentrations in brain that were >>10×EC₅₀ of 0.4 μM.

At least two compounds, IND24 and IND81, have good drug-like properties and dosing regimens, and formulations for each have been identified that will permit proof-of-concept studies in prion-infected mouse models. These compounds are well tolerated in chronic studies in mice over a wide range of doses. Dosing regimens and formulations have also been defined that will permit chronic dosing of IND54304 as a positive control in the efficacy studies evaluating IND24 and IND81.

### D. Example 1

### Chemical Libraries and HTS

One possible therapy for prion diseases, including CJD, is a “cocktail” of drugs that reduces the expression of GPI-anchored PrP^{C} on the surface of neurons and decreases the level of PrP^{Sc} by either slowing formation or increasing clearance. This “cocktail” approach, targeting multiple mechanisms of action in a disease pathway, has been successfully used to treat many complex, chronic, and fatal diseases, including AIDS, hepatitis C virus, and cancers. The first critical requirement is the availability of suitable in-vitro and in-vivo assays to identify and test experimental compounds. Because our goal was to lower both PrP^{C} and PrP^{Sc} in a target-agnostic manner, we used cell-based assays for primary HTS.

Here we report results from HTS of 44,218 diverse chemical compounds in PrP^{C} assays. We identified nine new leads that reduce PrP^{C} levels in human neuroblastoma cells (IMR32) and eight new leads that reduce levels of PrP^{C} in human glioblastoma (T98G) cells. We also report the results from HTS of 51,118 compounds in the PrP^{Sc} assay. We used ELISA assays to identify and confirm hits by single point confirmation (SPC) for the PrP^{C} and PrP^{Sc} assays, where confirmation included calcein assays to assess cell viability. For 682 SPC hits identified in the PrP^{Sc} assays, full dose-titration (EC₅₀) curves were used to evaluate potency by ELISA, calcein, and Western immunoblotting. We discovered 14 and 13 new chemical leads in dividing and non-dividing mouse ScN2a-c13 cells, respectively, that demonstrated good potency and cell viability. From these combined 44 leads, we identified analogs by structure-activity relationship (SAR) for analog-by-catalog purchase and testing.

The 44,218 compounds (as 570 plates) used in HTS in the PrP^{C} assays were two ChemBridge sets: 23,818 from ChB-1 and 20,400 from ChB-2. The ChB-2 set was a custom “CNS Set” obtained directly from ChemBridge. The 51,118 compounds (as ˜650 plates) tested in HTS in the PrP^{Sc} assays were from ChemBridge (21,015; ChB-1) and SPECS (30,103) libraries available at the Small Molecule Discovery Center (SMDC) at the University of California San Francisco. These represent a diversity set from among a larger set curated by the SMDC, where we had access to a total of 3,014 plates in 96-well format (˜150,000 compounds). Primary HTS hits from all libraries were first confirmed by SPC, using the original screening stocks. Further evaluation of confirmed hits using dose-titration curves (EC₅₀) was accomplished using fresh powders purchased from the corresponding vendor. For SAR expansion, analogs of validated lead compounds were acquired from various vendors.

For the chemical analysis, we first analyzed our already screened in-house collection and compared it to each of the candidate libraries. Using a Daylight fingerprint, we computed the nearest neighbor of each compound in the candidate set to the in-house dataset, rejecting compounds that were more than 60% similar. The qualifying compounds were counted as a metric for the additional coverage of chemical space.

We then investigated whether there was any difference between the libraries at the molecular fragment level. We fragmented the molecules in three different ways, using the Molinspiration software package “mib”: by the flag “-r1” ring systems, the flag “-ringSystems”, and Murcko scaffolds using the flag “-scaffold”. In each case, we counted the number of each type of fragment that was present in the new databases but was missing in the previously screened collection as a metric for chemical fragment novelty.

For biological target novelty, we used SEA to predict the target of each ligand in each collection, based on targets represented by ligands in the ChEMBL medicinal chemistry database version O₂. We counted the number of targets that had at least one predicted active and at least 10 predicted actives for the previously screened collection. We then asked whether the candidate libraries provided coverage for any of the ChEMBL targets that did not have predicted ligands in the previously screened collection. We found no difference between the two libraries on this basis. Because we wanted to achieve maximum chemical and target diversity, we chose the larger set of compounds (39,838; ChB-2) for the second-round testing in the PrP^{C} assay in order to identify more hits and leads. In total, we tested 42,159 compounds in the PrP^{C} assays derived from ChB-1 (23,000) and half of the set from ChB-2 (20,000).

To decide which of the 3,014 plates available at the SMDC would be most useful to screen in the PrP^{Sc} assays, we sought to develop a metric that prioritized plates having compounds that were more likely to hit, and that were not narrowly focused in congeneric series. We assessed plates using two metrics: bioactivity and mutual dissimilarity. Plates were assigned one point for each compound that had at least one prediction of bioactivity using SEA based on ChEMBL release 01 and using an E-value cutoff of 10⁻¹⁰. To calculate dissimilarity, we used a modified version of the method of Voigt, Bienfait, Wang and Nicklaus⁴¹. For each plate, we sorted the molecules by ascending molecular weight and scored one point for any molecule that differed from all previously accepted compounds by a Tanimoto coefficient (Tc) of 0.7 or more, based on default Daylight fingerprints. The combined score (bioactivity+dissimilarity) was used to compute a combined figure of merit for each plate, which was used to rank them, and from which we selected 650 plates for HTS analysis.

When we ranked the 3,014 available plates (˜180,000 compounds) by their combined figure of merit including bioactivity and mutual dissimilarity, we found the highest scoring compounds derived from 650 plates primarily from the ChB-1 and SPECS chemical libraries. We therefore screened those 650 plates first in the PrP^{Sc} assays. The ChB-2 library was not available to us when we ran the PrP^{Sc} HTS.

Each compound on each plate was represented as a simplified molecular input line entry specification (SMILES) string. In the case of salts and mixtures, we removed all but the largest organic molecule. We computed the biological targets for each molecule using SEA.

The source and number of compounds screened in each assay are summarized (Table 1). Not all compounds screened in the PrP^{C} assays were screened in the PrP^{Sc} assays.

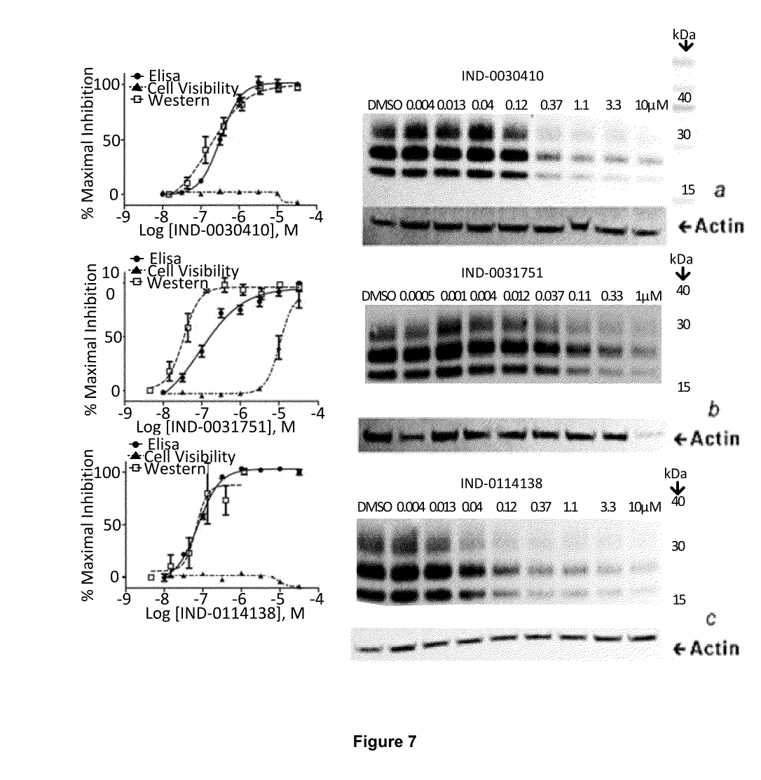

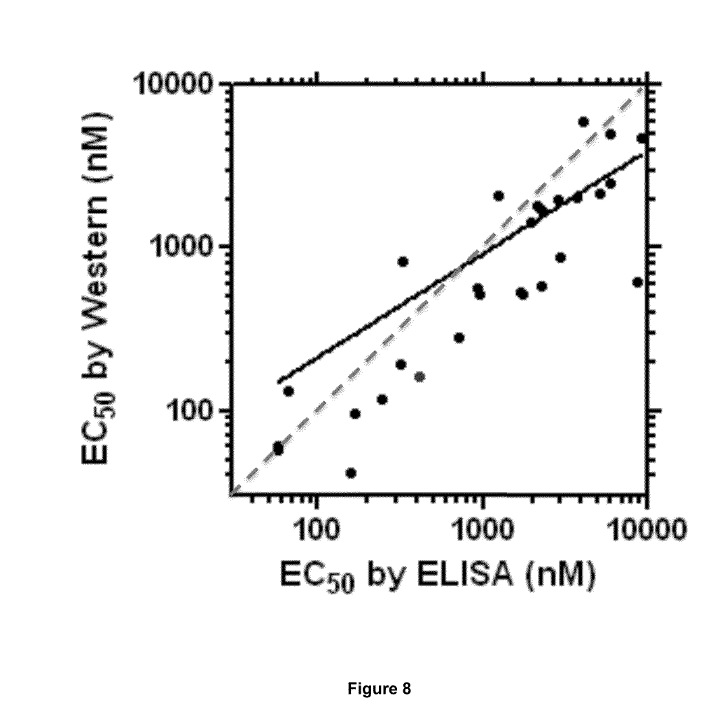

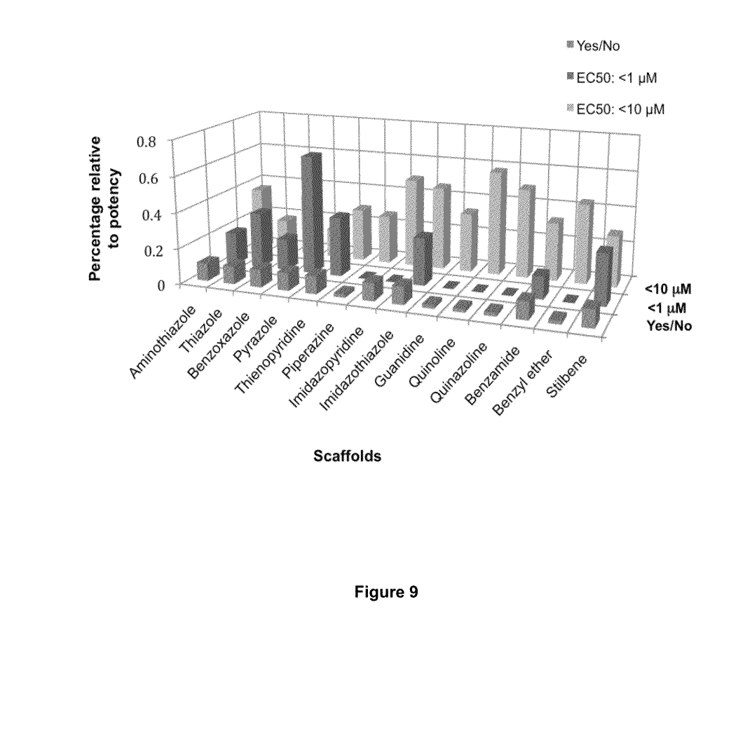

We initially selected 682 compounds and evaluated their antiprion potency and cell viability. About half of these were commercially available analogs, which were selected based on chemical similarity to HTS hits and tested to confirm the validity of the putative leads. Comparison of EC₅₀ (Western blot and ELISA data) and LC₅₀ (calcein AM results) curves are illustrated for three potent leads from the 682 tested (FIG. 7). Twenty-eight potent antiprion compounds representing 14 scaffolds in dividing cells were evaluated by dose-titration for PrP levels in Western immunoblots (FIG. 8). We found a strong correlation (R²=0.75; p<0.001) between ELISA and Western immunoblot data for antiprion potency for the 28 compounds tested (FIG. 8). The EC₅₀ results by scaffold and potency (<1 μM, 1-10 μM) are shown in Table 3. Structures, potency, and physicochemical properties for all 28 compounds are shown in Table 4. Potency by scaffold from Table 3 is graphed in relation to co-planarity and as a percentage of the total number of compounds (FIG. 9). The objective was to use all of the EC₅₀ results to develop SAR for drugs that would lower PrP^{C} and PrP^{Sc}.

### E. Example 2

### PrP^{C}

Seeking to extend our screening collection in the PrP^{C} assay, we evaluated two commercially available (ChemBridge) preplated collections for their ability to complement the ChB-1 library we had already screened. The two libraries were termed the “1.0 μL” set of 14,240 compounds and the “0.5 μL” set of 39,838 compounds. We evaluated the library based on one traditional criterion, the coverage of chemical fragment space, and one non-traditional one, the coverage of predicted biological target space, as predicted by the Similarity Ensemble Approach (SEA).⁴⁰

IMR32 and T98G human neuroblastoma and glioblastoma cells, respectively (ATCC CCL-127 and CRL-1690), were maintained in tissue culture flasks (175 cm²) containing 32 mL of supplemented MEM (without Geneticin). The cells were released from the flasks and seeded onto plates as described below for the ScN2a-c13 cells; 10,000 cells were added to each well of a white, clear-bottom, 96-well plate (Greiner) and allowed to incubate overnight at 37° C. The next day, test compounds (prepared as described below for the PrP^{Sc} ELISA) were added to each well and the plates returned to the incubator. After 2 days, the growth medium was aspirated, and each well washed once with PBS supplemented with 0.25 mg/mL BSA (wash buffer) and aspirated dry. IMR32 cells were fixed by the addition of 50 μL/well of 4% paraformaldehyde (in PBS); T98G cells were used without fixation by paraformaldehyde. After 20 min at room temperature (RT), the paraformaldehyde was removed by three washes of 250 μL/well of PBS and the wells aspirated dry. Horseradish peroxidase (HRP) conjugated anti-human PrP^{C} P antibody³⁹ (100 μL of a 1:1000 dilution in PBS supplemented with 3% w/v nonfat milk) was added to each well and the plate incubated at RT for 1 h. The antibody was removed with 5-6 washes of buffer (300 μL/well/wash), then 50 μL of Supersignal ELISA Pico Chemiluminescent substrate (Pierce Thermo) added to each well and the luminescence at 425 nm read immediately using a Spectramax M5 plate reader.

We screened all 23,858 small molecule compounds in the ChB-1 set first. We then analyzed the 24,000 (1.0 μL set) and 39,840 (0.5 μL set) small molecule compounds in the ChB-2 set for chemical fragment and biological target novelty to determine which would be screened next. We evaluated the 1.0 μL and 0.5 μL collections to select plates for screening. At the whole molecule level, we found that 1,691 and 4,371 of the 1.0 μL and 0.5 μIL collections, respectively, were more than 60% different from their nearest neighbor in the previously screened collection. This demonstrated that the 0.5 μL collection had nearly three times as many significantly new compounds as the 1.0 μL collection, roughly proportional to its three times larger size. At the fragment level, there were three different fragment types: the “-r1” ring systems, Murcko scaffolds, and small ring systems. We found that the 0.5 μL set had 17,698 new fragments, whereas the 1.0 μL collection had only 8,280 new fragments. Considering Murcko scaffolds, the 0.5 μL set had 8,030 new scaffolds, whereas the 1.0 μL set had 3,735 new scaffolds. Finally, considering the smallest ring systems, the 0.5 μL set had 304 new rings, whereas the 1.0 μL set had only 157 new rings. Based on all measures of chemical fragment novelty, the 0.5 μL set provided at least twice the novelty of the 1.0 μL set. Therefore, we used the 0.5 μL set for the second round of screening in the PrP^{C} assays and screened 20,000 compounds. This was called the ChB-2 set of compounds (Table 1). Together, the ChB-1 and ChB-2 sets were made up of 570 plates. Because we lacked sufficient drug, we did not screen the SPECS set in the PrP^{C} assay.

Considering the coverage of biological target space as predicted by SEA, we determined which of the 1,652 ChEMBL targets, for which ligands are known, had no predicted compounds in the ChB-1 and ChB-2 collection. We then did the same calculation against the two ChemBridge sets, to investigate whether they would provide useful additional coverage of biological target space. We found that the ChB-1 and ChB-2 sets covered 1,553 targets in ChEMBL with at least one compound. Including either ChemBridge set added effectively nothing to the biological target space coverage.

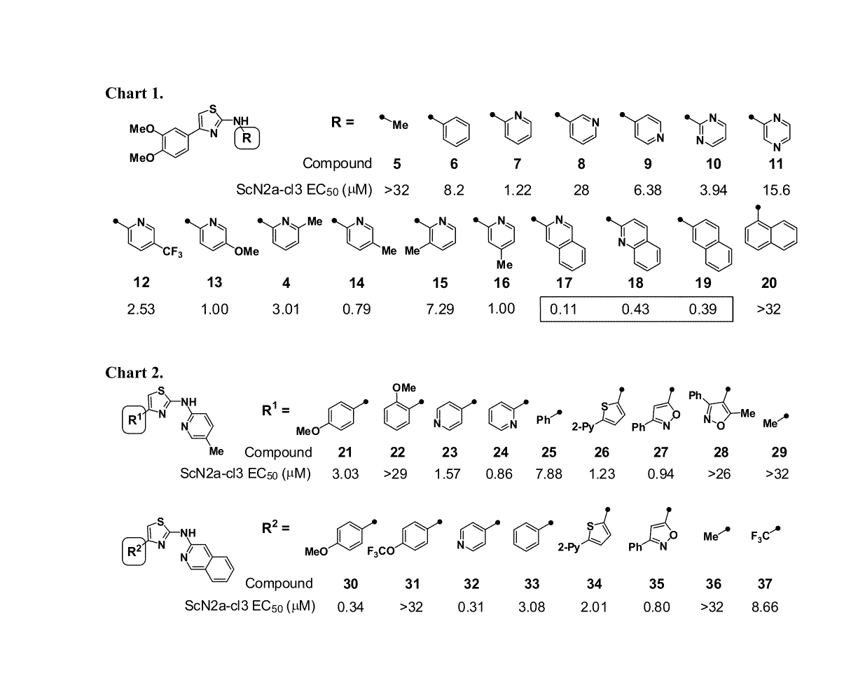

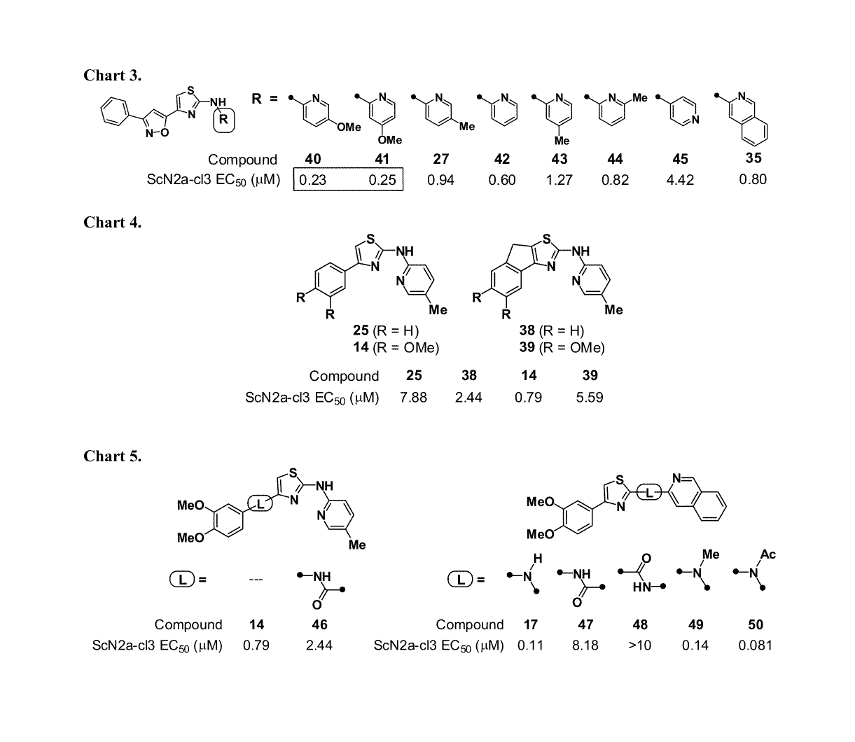

To establish the antiprion activity and general cytotoxicity of the commercial compounds, we used dose-response curves to calculate EC₅₀ and LC₅₀ (50% lethal concentration) values for PrP^{C} in IMR32 neuroblastoma and T98G glioblastoma cells. Z-scores for 190 runs was excellent in both cells lines, ranging from 0.6-0.95 (FIG. 1). For IMR32 and T98G cells, 675 and 579 HTS hits, respectively, were identified. Of 1,051 SPC assays performed, 239 and 277 hits were found for IMR32 and T98G cell lines, respectively. SPC hits led to the identification of 9 chemical scaffolds for IMR32 cells (FIG. 2) and 8 chemical scaffolds for T98G cells (FIG. 3). Seven scaffolds were shared by both cells lines. The distribution of PrP^{C} depletion, by scaffold and number of compounds screened within each scaffold, are shown in FIGS. 2 and 3. Two representative structures for 10 identified scaffolds (7 shared, 3 unique), along with molecular weights, SPC values, and calcein results are shown (Table 2). Confirmation of the SPC hits is currently ongoing; to date, 123 hits have been confirmed. One of the hits (IND-0061769) demonstrated EC₅₀ values of 2.05 μM and 1.84 μM for IMR32 and T98G cells, respectively.

Preliminary SAR analysis of the SPC hits in the PrP^{C} using IMR32 cells identified nine possible chemical classes including scaffolds similar to PrP^{Sc} leads such as AMT (2-aminothiazole), amide and quinoline scaffolds. New scaffolds such as sulfonamide, indole and ethanolamine were also found. From SAR analysis of SPC hits in PrP^{C} assays using T98G cells, eight scaffolds representing amide, sulfonamide, AMT, indole, chromene, quinoline, piperazine, and urea were found. Six scaffolds-amide, sulfonamide, fused indole, chromene, quinoline, and piperazinewere hits for both IMR32 and T98G cells. Further confirmation in the EC₅₀ assay will be conducted using SAR-by-catalog strategy on selected SPC hits from these scaffolds.

### F. Example 3

### PrP^{Sc}

The methods employed to evaluate the effects of compounds on PrP^{Sc} levels and cell viability were similar to previously published protocols²⁰ with the following modifications. ScN2a cells (N2a cells infected with the Rocky Mountain Laboratory prion strain) were seeded into black wall, clear bottom, tissue culture treated plates (Greiner) at either 40,000 cells/well (in 100 μL of assay medium: MEM supplemented with 10% FBS, GlutaMax and 500 μg/mL geneticin) for dividing cell assays or 150,000 cells/well (in assay medium +7 mM sodium butyrate to arrest cell division) for non-dividing cell assays. Compounds were dissolved in 100% DMSO and diluted in assay medium at 2× final concentration before addition to the assay plates (0.5% final DMSO concentration). Compound addition occurred 4 hours (dividing cells) or 24 hours (non-dividing cells) after cell seeding into the assay plates. After 5 days incubation at 37° C. in a humidified and 5% CO₂-enriched environment, lysates were generated as previously described²⁰ and transferred to high binding ELISA plates (Greiner) coated with D18 primary antibody for overnight incubation at 4° C. The next day, the plates were washed 3 times with TBST before addition of 100 μL of a 1:1000 dilution of HRP-conjugated D13 antibody in 1% BSA/PBS for a 1 hour incubation at room temperature. After incubation with the D13 antibody, the plates were washed seven times with TBST, 100 μL of ABTS was added to each well for 10 minutes and absorbance at 405 nm was read using a SpectraMax M5 plate reader (Molecular Devices, Sunnyvale, Calif.). Calcein cell viability assays were run on separately seeded 96 well black wall plates as previously described.¹

Mouse N2a neuroblastoma cells (ATCC) were transfected with full-length mouse PrP and infected with the Rocky Mountain Laboratory strain of mouse-adapted scrapie prions, yielding ScN2a-c13 cells (22). ScN2a-c13 cells were maintained in tissue culture flasks (175 cm²) containing 32 mL of filter-sterilized (0.2 μm) MEM with Earle's salts and L-glutamine, supplemented with 10% FBS, 250 μg/mL Geneticin, 50 I.U./mL penicillin and 50 μg/mL streptomycin (supplemented MEM) in a humidified and CO₂-enriched (5%) environment at 37° C. On day 1, the growth medium (supplemented MEM) was aspirated from the flasks, the cells washed twice with 10 mL of calcium- and magnesium-free Dulbecco's PBS, and then detached by addition of 3 mL of Cell Dissociation Buffer after incubation at RT for 5 min. The dissociation buffer was aspirated and the cells suspended in 10 mL of growth medium before counting using a Cellometer Auto T4 (Nexcelom Biosciences; Lawrence, Mass.). ScN2a-c13 cells were seeded either into new, 175-cm² tissue culture flasks for continued cell culture (9×10⁶ cells into 32 mL growth medium) or into 96-well, tissue-culture-treated, white polystyrene plates (Greiner Bio-One; Monroe, N.C.) for treatment with test compounds (40,000 cells/well in 100 μL of growth medium for dividing ScN2a cells; 150,000 cells/well in 80 μL of growth medium for stationary ScN2a cells). Stationary (nondividing) ScN2a cells were allowed to adhere for 1 h at 37° C. before cell division arrest was induced by addition of 20 μL of 35 mM sodium butyrate in growth medium (7 mM final concentration) and the plates incubated for 24 h prior to compound addition. Dividing ScN2a cells were allowed to adhere for 4 h at 37° C. before compound addition. Test compounds (100 μL) were added to each well to attain a final concentration of 10 μM. Three positive controls were used: simvastatin, quinacrine, and PAMAM-G4. Simvastatin and quinacrine (2 mM in 100% DMSO) were added, then diluted to a final concentration of 20 μM in growth medium (0.2% DMSO, final concentration). PAMAM-G4 was diluted from a 1% stock solution (in MeOH) to achieve a final concentration of 10 μg/mL. As a negative control, 0.2% DMSO in growth medium was used. Media were aspirated on day 5, and cells were washed with PBS (250 μL/well) and aspirated dry. The cells were lysed by addition of 20 μL of lysis buffer (10 mM Tris HCl, 150 mM NaCl, 0.5% sodium deoxycholate, 0.5% NP-40) containing 7.5 U/mL benzonase; plates were placed on a shaker at 37° C. for 1 h. Proteinase K [PK; 5 μL of 125 μg/mL in a Tris buffer (10 mM Tris HCl, 20 mM calcium chloride, 50% glycerol)] was added and incubated at 37° C. for 1 h, with shaking PK digestion was stopped by addition of 5 μL of cold (4° C.) 20 mM PMSF in ethanol. After 10 min at RT, 10 μL of 5 M guanidine isothiocyanate was added at 37° C. for 1 h (with shaking) to denature the protein. The lysate in each well was diluted with 120 μL of PBS, and 150 μL from each well transferred to 96-well polystyrene ELISA plates previously coated with D18 antibody (5 μg/mL/well in 300 μL of acidified PBS overnight at RT in a humidified chamber⁴²), the plates sealed, and incubated overnight at 5° C. The next day, the plates were washed 3 times with TBST buffer (20 mM Tris HCl, 137 mM NaCl, 0.05% Tween-20, pH 7.5), the contents of each well aspirated completely. Then 100 μL of a 1:1000 dilution of HRP-conjugated D13 antibody was added and incubated at 37° C. for 1 h. The plates were washed 4 times with TBST buffer, the contents aspirated completely, and 100 μL of ABTS peroxidase substrate added to each well. After 15 min of development at RT, the enzymatic reaction was stopped by addition of 100 μL of ABTS stop solution and the plates immediately loaded onto a SpectraMax M5 plate reader (Molecular Devices; Sunnyvale, Calif.) for measurement of absorbance at 405 nm.

ScN2a-c13 cells, cultured as described above, were seeded onto 6-well, tissue culture treated dishes at a density of 1.54×10⁶ cells/well in 6.2 mL of supplemented MEM and allowed to adhere for 4 h at 37° C. Test compounds diluted in supplemented MEM (described above) were added to the plate (0.8 mL/well) to attain final concentrations ranging from 1 nM to 32 μM. After 5 days, the media was aspirated from each well and the plates washed one time with PBS (7 mL/well). The cells were lysed by addition of 0.35 mL of lysis buffer (20 mM Tris HCl, pH 8.0; 100 mM NaCl; 0.5% NP-40; 0.5% sodium deoxycholate; and 7.5 U/mL benzonase). The total protein in the lysate was measured using a bicinchoninic protein assay (Pierce). A total of 0.06 mg protein was treated with PK (total protein:enzyme ratio=50:1) in 0.1 mL PBS and the sample incubated at 37° C. for 1 h. The proteolytic digestion was terminated by the addition of PMSF to a final concentration of 3 mM. The samples were centrifuged at 16,000×g for 1 h, the supernatant discarded, and the pellets resuspended in 15 μL of reducing SDS sample buffer. The PrP^{Sc}-containing samples were denatured by heating at 80° C. for 5 min and run in a 4-12% Tris glycine SDS gel (Invitrogen). The gels were transferred to nitrocellulose membrane using a Nupage apparatus (Invitrogen) and the membranes blocked with 5% (w/v) nonfat milk/TBST overnight. The membranes were immersed in a 1:10000 dilution of D13-HRP antibody (1 mg/mL) for 1 h at RT, washed 3 times with TBST buffer before development (1 min) with Enhanced Chemiluminescent Western Blot reagent (GE Healthcare). Imaging and quantification of the blots were done using a Gene Gnome (Syngene) equipped with Gene Tools software.

To quantify actin, 200 μg total protein from lysate was diluted 1:5 with cold ethanol and incubated overnight at −20° C. The next day, the sample was centrifuged for 60 min at 16,000×g, the protein pellet dried and dissolved in Nupage loading buffer. The sample was loaded onto a 4-2% BisTris gel system (Invitrogen), transferred to a nitrocellulose membrane, and blocked with 5% (w/v) nonfat milk/TBST for 30 min at RT. The membranes were immersed in a 1:10,000 dilution of rabbit anti-actin polyclonal antibody (Sigma) in 5% (w/v) nonfat milk/TBST for 1 h, and then washed three times with TBST buffer. The membranes were then incubated with a 1:10,000 dilution of goat anti-rabbit HRP-conjugated secondary antibody (Biorad) in 5% (w/v) nonfat milk/TBST for 1 h, washed three times with TBST buffer, and developed with Enhanced Chemiluminescent Western Blot reagent. Imaging and quantification were performed as described above.

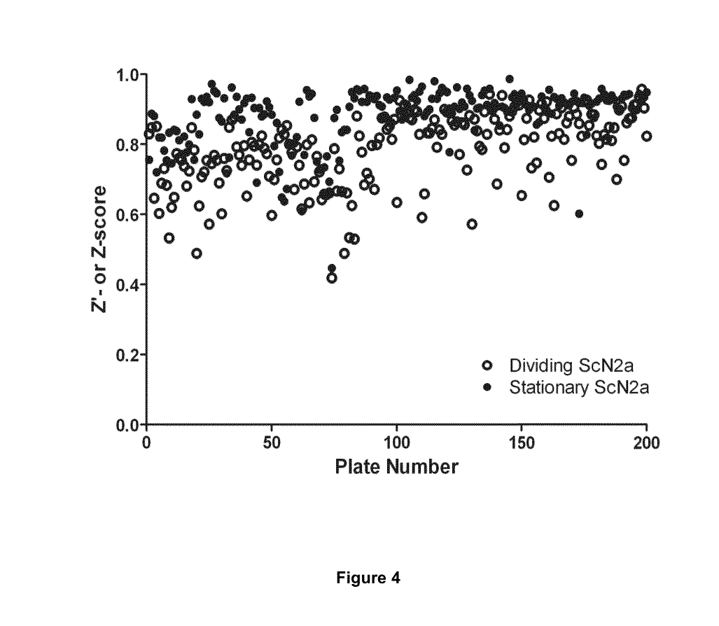

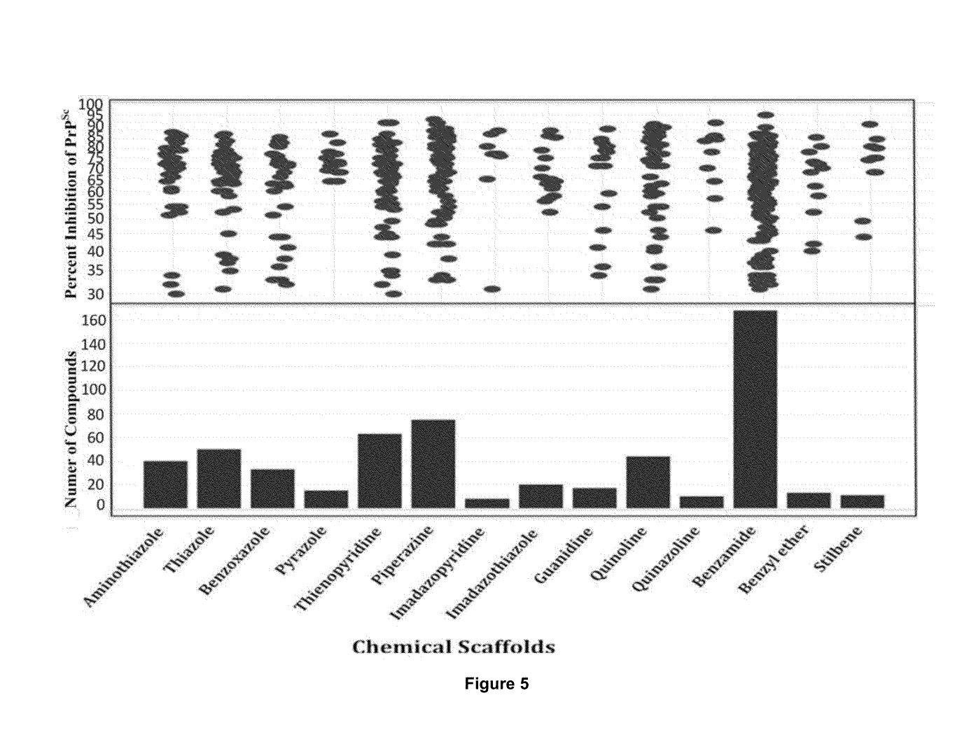

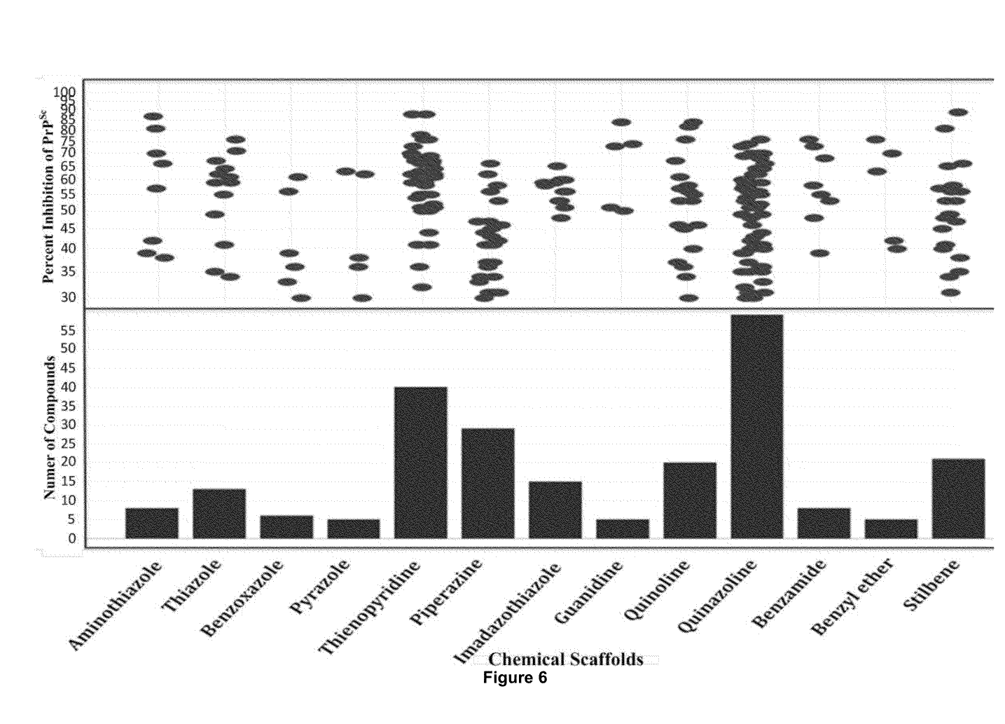

To establish the antiprion activity and general cytotoxicity of the compounds, we used dose-response curves to calculate EC₅₀ and LC₅₀ values in both nondividing and dividing ScN2a-c13 cells. The number of cells remained unchanged (3,500,000) in the presence of 7 mM sodium butyrate (NaB) over the course of 3 days, where percent viability remained unchanged, arguing that cells were not dividing. In contrast, cell numbers at 3 days were significantly higher (10,000,000; p <0.05) when no NaB was added. Z′ and Z scores over 200 runs were excellent for both dividing and nondividing assays, ranging from 0.5-0.9 (FIG. 4). From the 650 plates, 3,100 HTS hits were found for dividing cells and 320 HTS hits were obtained for nondividing cells. Of the 3,100 hits from dividing cells, 2,033 compounds were selected for the SPC assay, which yielded 970 hits and 381 hits in dividing and nondividing cells, respectively. From the 1,351 hits by SPC (dividing and nondividing cells), we identified 14 chemical scaffolds in dividing cells (FIG. 5) 13 chemical scaffolds in nondividing cells (FIG. 6), after eliminating those that demonstrated cytotoxicity. Two hundred twelve compounds were identified in both dividing and nondividing ScN2a-c13 cells. We identified one additional scaffold, imidazopyridine, among the hits in dividing ScN2a-c13 cells. Assays are underway to confirm the remaining 108 molecules from the 320 HTS hits in nondividing cells (less the 212 the overlapped with dividing cells).

Using the 970 SPC hits from dividing ScN2a-c13 cells, we performed SAR analysis and identified 20 chemical scaffolds. From these, we selected 14 scaffolds for further assessment of their antiprion potency and cell viability in dose-response assays. Six scaffolds were excluded because of unacceptable chemical properties or close similarity to other scaffolds. A SAR-by-catalog strategy was initiated; the first set of 467 compounds was obtained and their EC₅₀ values were measured to confirm potency and establish preliminary SAR around these chemical scaffolds. We identified two scaffolds (thienopyridine and benzamide) with many compounds having EC₅₀ values of <1 μM (Table 3 and FIG. 9). Several scaffolds, including piperazine, imidazolopyridine, guanidine, quinoline, quinazoline, and benzyl ether, demonstrated low antiprion potencies. For these less active scaffolds, it is possible that the analogs selected were suboptimal and do not represent the true potential of these scaffolds.

Upon closer examination of the hits across different chemical scaffolds, several trends became evident. A conjugated aromatic or heteroaromatic ring system was prominent in all lead structures. This ring system is comprised of more than two aryl or heteroaryl groups joined in a fused or linear fashion. In the case of linear aromatic systems, two aromatic rings can be linked directly via a carbon-carbon bond or a linker such as an amide or double bond. Further analysis revealed that compounds with better potency (<1 μM) were from scaffolds having core structures possessing a flat coplanar or near coplanar conformation, such as the AMT, thiazole, benzoxazole, pyrazole, thienopyridine, imidazothiazole, benzamide, and stilbene scaffolds. In contrast, scaffolds with rings connected via flexible groups that disrupt coplanarity of the aryl ring system had diminished antiprion activity (>1 μM).

From initial SAR observations, we selected the thienopyridine scaffold for further analysis. We used 80 thienopyridines and determined their EC₅₀ and LC₅₀ values in dividing ScN2a-c13 cells (selected analogs shown in Table 5). For the amide R² group, a phenyl ring with one substituent seemed to be optimal for antiprion activity (IND-0035860 and IND-0037769) (Table 5). In the unknown target, the space around the phenyl group of the amide may be restricted based on the diminished antiprion activity of a larger congener IND-0035833 containing a dioxane ring. A similar trend was also observed with the thiophene series (R¹⁼thiophene, IND-0024576), for which the antiprion potency tended to decrease as the size of the substituent on the amide phenyl ring increased. In support of this trend, the congener IND0042103 bearing the largest R² group was inactive in our assay. Also, aliphatic amides with flexible methylene linkers were less favorable (IND-0040541 and IND-0037771) compared to aryl amides from anilines.

With respect to the R¹ substitution at the C-6 position of the thienopyridine scaffold, a thiophene or phenyl ring may be preferable. It is also clear that the congener with one methoxy group on the phenyl ring (IND-0035860) was more potent than the corresponding congeners with two substituents (IND-0044746 and IND-0042063) (Table 6). The thiophene analog (IND-0024576) was equipotent to the phenyl analog (IND-0035860) (Table 5). However, the electron-deficient pyridyl ring at R¹ position was less tolerated (compare IND-0024575 with IND-0024581) (Table 6).

It is noteworthy that IND-0052025 with a phenyl ring fused to the thienopyridine scaffold had similar potency to the unfused analog IND-0037769; however, the cyclohexyl-fused ring system (IND-0024609) was inactive, suggesting a flat structure is preferred at this position (Table 6).

### G. Example 6

### Cell Viability Assays

Human IMR32 or human T98G, and mouse N2a-c13 cells were seeded into 96-well, black polystyrene plates (Greiner) and treated with compound as described above for the ELISA plates. After 2 (IMR32 and T98G) or 5 days (N2a-c13), the growth media was aspirated, the plates washed once with PBS (250 μL/well), and the plates aspirated dry. Calcein-AM (100 μL/well, 5 μg/mL solution in calcium- and magnesium-free PBS) was added, and the plates were incubated at 37° C. for 45 min. Fluorescent emission intensity was quantified using a Spectramax M5 plate reader, excitation/emission spectra of 485 nm/530 nm.

### H. Example 8

### Data Analysis

Absorbance (PrP^{Sc} ELISA), chemiluminescence (PrP^{C} ELISA), and fluorescence (calcein cell viability) data were exported from the plate readers as text files and normalized either to positive controls (PAMAM-G4 for PrP^{Sc}, quinacrine for calcein) or to background (PrP^{C}). Data were processed and stored in a Collaborative Drug Discovery (CDD) web-based database. Inhibition curves were generated using nonlinear regression employing the Levenberg Marquardt algorithm programmed into the CDD database.⁴³ Results were analyzed using EXPLORE DATA and SEARCH BY PROTOCOLS, where for each protocol, RUN date or a date range and an assay READOUT or READOUT range was defined with or without the limit of a specific READOUT or READOUT range (e.g., EC₅₀ value between 1 to 10 μM). Searches were performed by using a combination of two or more assay protocols based on chemical structures and molecule ID numbers using STRUCTURE EDITOR and MOLECULE KEYWORD EDITOR. Search results were exported to Excel as structure data files (SDF) or comma-separated values (CSV) files for further data manipulation and SAR analysis.

### I. Example 9

### Analyzing Assay Performance

Z′ and Z scores to assess the precision, accuracy, and robustness were calculated using the following equations:⁴⁴

Z′=1−[3(s.d._{pos.control}+s.d_{neg.control})]/|mean_{pos.control}−mean_{neg.control}|

Z=1−[3(s.d._{background}+s.d_{neg.control})]/|mean_{background}−mean_{neg.control}|

Z′=1−[3× standard deviation for the positive control value+the standard deviation for the negative control] divided by the absolute value of the difference between the mean of the positive control−the mean of the negative control.
Z=same as equation above, except replace the positive control value measured in the equation with the value measured for background.

for which background is the chemiluminescence signal in the absence of HRP-conjugated P antibody.

### J. Example 10

### Structure-Activity Relationships

SAR analyses were performed using SARvision (Altoris Inc.), permitting the visualization, mining, and organization of chemical data. Chemical structure and biological assay data were combined in sdf files. SARvision was used to generate a list of scaffold(s) and organize them into hierarchical tree structures using the IDENTIFY SCAFFOLDS feature. Scaffolds were also drawn manually by selecting DRAW SCAFFOLD under the TREE dropdown menu. Additions or deletions by column/row were accomplished by selecting the appropriate function item under the TABLE dropdown menu, specifically to filter data by scaffold type or any associated data, such as HTS results, SPC data, EC₅₀ values and physicochemical information. SARvision was also used to generate R-group tables to better visualize SAR for each chemical scaffold. The sorted data and table were then exported to MS Word, MS Excel, plain text, HTML, and sdf formats.

### K. Example 10

### Physicochemical Parameters

Qikprop (Schrödinger, New York, N.Y.)⁴⁵⁻⁴⁶ was used to estimate a variety of pharmaceutically relevant physiochemical properties, including calculated log of octanol:water partition coefficient (clogP), polar surface area (PSA), log blood-brain barrier (logBBB) permeability, Caco-2 and MDCK cell permeability, and the number of hydrogen bond acceptors (HBA) and donors (HBD).

### L. Example 11

### Compounds Reducing Both PrP^{C} and PrP^{Sc}

Because a potentially ideal treatment for prion diseases would include drugs that lower levels of PrP^{C} and PrP^{Sc} (by decreasing its formation and/or increasing its clearance), we determined if any compounds were identified in more than 1 of our 4 assays (PrP^{C} in IMR32 cells, PrP^{C} in T98G cells, PrP^{Sc} in dividing ScN2a-c13 cells, PrP^{Sc} in nondividing ScN2a-c13 cells) (FIG. 10). Sixteen compounds lowered PrP^{C} (either IMR32 or T98G cells) and reduced PrP^{Sc} in stationary cells, which model nondividing neurons in human adults. One compound [IND-0001270, a tetrahydroquinoline] was identified as active in all 4 assays.

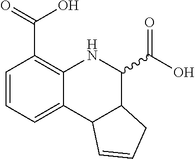 [SMILES: O=C(O)c1cccc2c1NC(C(=O)O)C1CC=CC21]

### 3a, 4, 5,9b-tetrahydro-3H cyclopenta[c]quinoline-4,6-dicarboxylic acid

### M. Example 12

### Materials and Methods for Chemical Ananlysis and In Vitro and In Vivo Studies

General Reagents and solvents were purchased from Aldrich Chemical, Acros Organics, Alfa Aesar, AK Scientific, or TCI America and used as received unless otherwise indicated. Air and/or moisture sensitive reactions were carried out under an argon atmosphere in oven-dried glassware using anhydrous solvents from commercial suppliers. Air and/or moisture sensitive reagents were transferred via syringe or cannula and were introduced into reaction vessels through rubber septa. Solvent removal was accomplished with a rotary evaporator at ca. 10-50 Torr. Automated column chromatography was carried out using a Biotage SP1 system and silica gel cartridges from Biotage. Analytical TLC plates from EM Science (Silica Gel 60 F₂₅₄) were employed for TLC analyses. Melting points were determined with an electrothermal capillary melting point apparatus and are uncorrected. ¹H NMR spectra were recorded on a Varian INOVA-400 400 MHz spectrometer. Chemical shifts are reported in δ units (ppm) relative to TMS as an internal standard. Coupling constants (J) are reported in hertz (Hz). Characterization data are reported as follows: chemical shift, multiplicity (s=singlet, d=doublet, t=triplet, q=quartet, br=broad, m=multiplet), coupling constants, number of protons, mass to charge ratio.

Minimum Essential Medium (MEM), Geneticin, Dulbecco's phosphate-buffered saline (PBS), Tris HCl, proteinase K, glycerol, SDS sample buffer and calcein-AM were purchased from Invitrogen (Carlsbad, Calif.); fetal bovine serum (FBS) from Thermo Scientific Hyclone (Rockford, Ill.); penicillin and streptomycin from Cellgro (Manassas, Va.); Cell Dissociation Buffer from Millipore (Billerica, Mass.); NaCl, ABTS peroxidase substrate and ABTS stop solution from Fisher Chemical (Houston, Tex.); ethyl alcohol from Gold Shield Chemical Co. (Hayward, Calif.); benzonase from EMD chemicals (Gibbstown, N.J.); phenylmethylsulfonyl fluoride (PMSF) from MP Biomedicals (Solon, Ohio); and guanidine isothiocyanate from RPI (Mt. Prospect, Ill.). D18 and D13 antibodies were obtained as previously described.³⁹ All other compounds and reagents were purchased from Sigma (St. Louis, Mich.) unless otherwise specified below.

Dose formulations for in vivo pharmacokinetic studies contained propylene glycol (Sigma-Aldrich, St. Louis, Mo.), absolute ethanol (Fisher Scientific, Pittsburg, Pa.), labrosol (Gattefosse, France), and polyethylene glycol 400 (Hampton Research, Aliso Viejo, Calif.). Brain tissue was homogenized using a Precellys 24 (Bertin Technologies, France) tissue homogenizer. LC/MS/MS analysis was performed using an API 4000 triple quadruple mass spectrometer (Applied Biosystems) with Analyst 1.4.2 software, coupled to a Shimadzu CBM-20A controller, LC20AD pumps, and SIL-5000 auto sampler (Shimadzu Scientific, Columbia, Md.). Compounds were separated on a Gemini C18, 3 μm, 50×2 mm column (Phenomenex, Torrance, Calif.) using a gradient between 0.1% formic acid in water and 0.1% formic acid in acetonitrile.

For in-vivo pharmacokinetic studies, compounds were dissolved in a formulation containing 20% propylene glycol, 5% ethanol, 5% labrosol, and 70% polyethylene glycol 400 (PEG400) and administered by oral gavage to female FVB mice weighing ˜25 g. At specified time points after dosing (0.25, 0.5, 1, 2, 4, 6, and 24 h), 2 animals were euthanized by CO₂, and ˜1 mL blood (by cardiac puncture) and brain samples were collected. The heparinized blood samples were centrifuged to obtain plasma, which was stored at −80° C. until analysis. Brain samples were weighed, diluted 4-fold with water, and then homogenized using a Precellys 24 tissue homogenizer. Brain homogenates (20% wt/vol) were stored at −80° C. until analysis. Pharmacokinetic protocols involving animals were all reviewed and approved by the UCSF institutional animal care and use committee (IACUC).

Plasma and brain homogenate samples were extracted using a protein-precipitation method and analyzed by specific LC/MS/MS methods developed for each compound dosed in vivo. The analytical method accuracy and precision were monitored by analyzing quality control (QC) samples that were prepared and treated using the same methods as calibration standards for the plasma or brain homogenate samples.

The data were used to calculate the area under the concentration-time curve (AUCₗₐₛₜ) by noncompartmental analysis with sparse sampling performed using Phoenix WinNonlin 6.1 software (Pharsight, Mountain View, Calif.).

Thirty-two compounds from scaffolds that showed good potency (EC₅₀<1 μM) in the in-vitro screening assays and predicted to have good blood-brain barrier penetration properties were screened in vivo. Compounds were dosed by oral gavage at 10 mg/kg in female FVB mice, and then brain and plasma concentrations measured at various time points after dosing. Many compounds showed higher concentrations in brain compared to plasma; two compounds representing three scaffolds are shown (as measured by AUCₗₐₛₜ values) (Table 7 and FIG. 11).

All 235 2-AMT analogs were synthesized at small scale (up to 1 g) at the Small Molecule Discovery Center at UCSF. Lead compounds IND24 and IND81 were subsequently synthesized at 100-200 g scales at ChemVeda (Hyderabad, India). One hundred mg of Compound B (IND54304) [(E)-5-(4-(2-(pyridin-4-ylmethylene)hydrazinyl)phenyl)oxazole] along with the synthetic scheme was generously provided by Professor Katsumi Doh-ura (Tohoku University, Sendai, Japan), subsequently synthesized at UCSF, and finally scaled-up to 100-g quantities at ChemPartner (Shanghai, China). Warfarin (positive control for protein binding assay) and chlorowarfarin (internal standard for warfarin) were obtained from Toronto Research Chemicals (Ontario, Canada). Blank sodium heparinized plasma from rat (Sprague-Dawley), dog (beagle), mouse (CD-1) and human was obtained from Bioreclamation (Hicksville, N.Y.), and Dulbecco's phosphate-buffered saline (PBS) from Invitrogen (Carlsbad, Calif.). The rapid equilibrium dialysis (RED) devices and reusable base plate were obtained from Thermo Scientific (Rockford, Ill.).

Pooled male and female Sprague-Dawley, CD-1, and beagle liver microsomes, and pooled human liver microsomes, 0.5 M potassium phosphate pH 7.4, and NADPH Regenerating System Solutions A and B were obtained from BD Biosciences (Bedford, Mass.). Dextromethorphan HBr (positive control for microsomal assay) was obtained from Sigma-Aldrich (St. Louis, Mo.), and d3-dextromethorphan (internal standard for dextromethorphan) was obtained from Toronto Research Chemicals (Ontario, Canada).

FVB mice (bred at the Hunter's Point animal facility at UCSF or purchased from Charles River, Hollister, Calif.) were used for all pharmacokinetic studies. Dose formulations for in vivo pharmacokinetic studies contained DMSO (Thermo Fisher Scientific, Rockford, Ill.), propylene glycol (Sigma-Aldrich, St. Louis, Mo.), absolute ethanol (Fisher Scientific, Pittsburgh, Pa.), labrosol (Gattefosse, France), and polyethylene glycol 400 (Hampton Research, Aliso Viejo, Calif.). Rodent liquid diet was obtained from Bio-Sery (Frenchtown, N.J.). Brain tissue was homogenized using a Precellys 24 (Bertin Technologies, France) tissue homogenizer. LC/MS/MS analysis was performed using an API 4000 triple quadruple mass spectrometer (Applied Biosystems) with Analyst 1.4.2 software, coupled to a Shimadzu CBM-20A controller, LC20AD pumps and SIL-5000 auto sampler (Shimadzu Scientific, Columbia, Md.). Compounds were separated on either a BetaBasic C18 or a BDS Hypersil C8 column (both 3 μm, 50×2 mm; Thermo Scientific, Rockford, Ill.) using a gradient between 0.1% formic acid in water and 0.1% formic acid in acetonitrile (ACN). HPLC-grade ACN and water were obtained from VWR Scientific (Radnor, Pa.).

### N. Example 13

### Chemical Purification and Analysis

All analogs submitted for testing (3-50) were judged to be of 95% or higher purity based on analytical LC/MS analysis. LC/MS analyses were performed on a Waters Micromass ZQ/Waters 2795 Separation Module/Waters 2996 Photodiode Array Detector system controlled by MassLynx 4.0 software. Separations were carried out on an XTerra® MS C₁₈ 5 μm 4.6×50 mm column at ambient temperature using a mobile phase of water-acetonitrile containing 0.05% trifluoroacetic acid. Gradient elution was employed wherein the acetonitrile-water ratio was increased linearly from 5 to 95% acetonitrile over 2.5 minutes, then maintained at 95% acetonitrile for 1.5 min., and then decreased to 5% acetonitrile over 0.5 min, and maintained at 5% acetonitrile for 0.5 min. Compound purity was determined by integrating peak areas of the liquid chromatogram, monitored at 254 nm.

The general procedure for synthesis of the 2-AMTs is depicted in Scheme 1. Details of the synthesis of the 34 2-AMTs are given below.

For LC/MS quantification for all 2-AMTs, samples and their respective internal standards were injected into either a BetaBasic C18 or BDS Hypersil C8 column. The solvent system used for separation was composed of water and ACN containing 1% formic acid. For quantification of IND24 and IND81, samples (along with a proprietary internal standard) were injected onto a BetaBasic C18 column maintained at room temperature. The amount of ACN in the gradient was increased from 75% ACN to 95% ACN over 2.5 min, held for 0.5 min, and then re-equilibrated to 75% ACN over 1.4 min. Data acquisition used MRM in the positive ion mode, and the transitions monitored were m/z 344→226 for IND24; m/z 351→233 for IND81; and m/z 363→245 for internal standard.

For quantification of IND54304, samples (along with a proprietary internal standard) were injected onto a BDS Hypersil C8 column maintained at room temperature. The amount of ACN in the gradient was increased from 25% ACN to 95% ACN over 2.0 min, held for 1.0 min, and then re-equilibrated to 25% ACN over 1.4 min. Data acquisition used MRM in the positive ion mode, and the transitions monitored were m/z 265→160 for IND54304 and m/z 321→253 for internal standard.

For quantification of warfarin, samples (along with chlorowarfarin as internal standard) were injected onto a BetaBasic C18 column maintained at room temperature. The amount of ACN in the gradient was increased from 70% ACN to 95% ACN over 1.8 min, held for 0.5 min, and then re-equilibrated to 70% ACN over 1.4 min. Data acquisition used MRM in the negative ion mode, and the transitions monitored were m/z 307→161 for warfarin and m/z 341→161 for chlorowarfarin.

For quantification of dextromethorphan, samples (along with d3-dextromethorphan as internal standard) were injected onto a BDS Hypersil C8 column maintained at room temperature. The amount of ACN in the gradient was increased from 50% ACN to 95% ACN over 2.5 min, held for 1.5 min, and then re-equilibrated to 50% ACN over 1.0 min. Data acquisition used MRM in the positive ion mode, and the transitions monitored were m/z 272>215 for dextromethorphan and m/z 275→215 for d3-dextropethorphan.

### O. Example 14

### Chemical Synthesis

The following compounds were obtained from commercial suppliers: [4-(4-Methoxy-phenyl)-thiazol-2-yl]-(4-methyl-pyridin-2-yl)-amine (IND2; ChemDiv, Inc., San Diego, Calif.), (6-Methyl-pyridin-2-yl)-(4-pyridin-4-yl-thiazol-2-yl)-amine (IND7) and 4-[2-(4-Methyl-pyridin-2-ylamino)-thiazol-4-yl]-phenol (IND49; ChemBridge, Inc., San Diego, Calif.), and (4-Methyl-pyridin-2-yl)-(4-pyridin-4-yl-thiazol-2-yl)-amine (IND52; ASINEX. Winston-Salem, N.C.).

The synthesis of the precursors (5-Methyl-pyridin-2-yl)-thiourea, (4-Methyl-pyridin-2-yl)-thiourea, Isoquinolin-3-yl-thiourea, (4-Methoxy-pyridin-2-yl)-thiourea, (5-Methoxy-pyridin-2-yl)-thiourea, and (6-Methyl-pyridin-2-yl)-thiourea, and of (5-Methyl-pyridin-2-yl)-(4-pyridin-4-yl-thiazol-2-yl)-amine (IND29), (5-Methyl-pyridin-2-yl)-[4-(3-phenyl-isoxazol-5-yl)-thiazol-2-yl]-amine (IND33), [4-(5-Methyl-3-phenyl-isoxazol-4-yl)-thiazol-2-yl]-(5-methyl-pyridin-2-yl)-amine (IND36), [4-(3,4-Dimethoxy-phenyl)-thiazol-2-yl]-(4-methyl-pyridin-2-yl)-amine (IND42), [4-(3,4-Dimethoxy-phenyl)-thiazol-2-yl]-(5-trifluoromethyl-pyridin-2-yl)-amine (IND43), [4-(3,4-Dimethoxy-phenyl)-thiazol-2-yl]-(5-methoxy-pyridin-2-yl)-amine (IND44), [4-(3,4-Dimethoxy-phenyl)-thiazol-2-yl]-isoquinolin-3-yl-amine (IND46), [4-(3,4-Dimethoxy-phenyl)-thiazol-2-yl]-quinolin-2-yl-amine (IND47), (5-Methyl-pyridin-2-yl)-[4-(5-pyridin-2-yl-thiophen-2-yl)-thiazol-2-yl]-amine (IND76), [4-(3,4-Dimethoxy-phenyl)-thiazol-2-yl]-pyrazin-2-yl-amine (IND82), Isoquinolin-3-yl-(4-pyridin-4-yl-thiazol-2-yl)-amine (IND85), Isoquinolin-3-yl-[4-(4-methoxy-phenyl)-thiazol-2-yl]-amine (IND86), (4-Methoxy-pyridin-2-yl)-[4-(3-phenyl-isoxazol-5-yl)-thiazol-2-yl]-amine (IND112), (5-Methoxy-pyridin-2-yl)-[4-(3-phenyl-isoxazol-5-yl)-thiazol-2-yl]-amine (IND120), and [4-(3,4-Dimethoxy-phenyl)-thiazol-2-yl]-pyridin-2-yl-amine (IND135) is described below.

General Procedure for Preparing Thiourea Intermediates from Amines.

Neat phenyl isothiocyanate (1.1 mmol, 1.1 equiv) is added dropwise to a stirred solution of the aniline, aminopyridine, or other amine building block (1 mmol) in acetone (10 mL) at room temperature. The reaction mixture is heated to reflux for 1-3 hours until judged complete (LC/MS), and then cooled, poured into water-ice, and stirred for an additional 30 min. The benzoyl thiourea precipitate is collected by filtration and washed with more water. This crude material is dissolved in methanol (20 mL) and treated with 5 mL of aqueous 1N NaOH. The reaction mixture is heated to 80° C. until hydrolysis is judged complete (LC/MS). After cooling, the reaction mixture is poured into water-ice and sufficient aqueous 1N HCl is added to produce a neutral (pH˜7) solution. The thiourea intermediate typically precipitates from the neutral solution and is collected by filtration and dried. This two-step procedure provides thiourea intermediates in 50-95% overall yield, with purities generally >90% as determined by ¹H NMR. These intermediates are used in the next step without further purification.

The general procedure for synthesis of the 2-aminothiazoles is depicted in Scheme 1.

General Procedure for Preparing 2-AMT Analogs from Thioureas.

An ethanolic solution of thiourea prepared above (1 mmol) and commercially available bromoacetophenones (1 mmol, 1.1 equiv.) was stirred at room temperature overnight. Once the reaction was done (as monitored by LC/MS) the reaction mixture was poured into water-ice (20 mL) and stirred for another 30 min. A saturated solution of Na₂CO₃ was added to produce a solution of pH ˜8. The aminothiazole product typically precipitated from this solution and was collected by filtration and washed with water. Crude aminothiazoles were purified by column chromatography on silica gel (˜40 to 100% ethyl acetate-hexane). Relevant fractions were collected and concentrated to afford the desired product in 60-95% yields, with purity of >95% as determined by 1H-NMR.

### [4-(3,4-dimethoxyphenyl)-thiazol-2-yl]-(6-methylpyridin-2-yl)-amine (4)

Intermediate 51 was reacted with commercially available 2-bromo-1-(3,4-dimethoxyphenyl)-ethanone according to the general procedure to afford the title compound in 92% yield; mp 264-266° C. ¹H NMR (DMSO-d₆) δ 11.41 (br. s., 1H, NH), 7.63 (t, J=7.78 Hz, 1H), 7.42-7.50 (m, 2H), 7.32 (s, 1H), 7.00 (d, J=8.42 Hz, 1H), 6.91 (d, J=8.24 Hz, 1H), 6.82 (d, J=7.33 Hz, 1H), 3.82 (s, 3H), 3.79 (s, 3H), 2.49 (s, 3H); LCMS (ESI) m/z 328 (MH+)

### 4-(3,4-dimethoxyphenyl)-N-methyl-1,3-thiazol-2-amine (5)

Methyl thiourea (Aldrich Chemical) was reacted with commercially available 2-bromo-1-(3,4-dimethoxyphenyl)-ethanone according to the general procedure to afford the title compound in 60% yield; mp 120-123° C. ¹H NMR (DMSO-d₆) δ 7.53 (d, J=4.76 Hz, 1H), 7.35-7.41 (m, 2H), 6.91-6.97 (m, 2H), 3.79 (s, 3H), 3.76 (s, 2H), 2.86 (d, J=4.76 Hz, 3H); LCMS (ESI) m/z 251 (MH+)

### [4-(3,4-dimethoxyphenyl)-thiazol-2-yl]pyridin-2-yl-amine (6)

Intermediate 62 was reacted with commercially available 2-bromo-1-(3,4-dimethoxyphenyl)-ethanone according to the general procedure to afford the title compound in 62% yield; mp 211-214° C. ¹H NMR (DMSO-d₆) δ 11.37 (s, 1H, NH), 8.27-8.33 (m, 1H), 7.66-7.74 (m, 1H), 7.43-7.50 (m, 2H), 7.31 (s, 1H), 7.09 (d, J=8.24 Hz, 1H), 6.99 (d, J=8.42 Hz, 1H), 6.92 (ddd, J=0.92, 5.08, 7.19 Hz, 1H), 3.82 (s, 3H), 3.78 (s, 3H); LCMS (ESI) m/z 314 (MH+)

### [4-(3,4-dimethoxyphenyl)-thiazol-2-yl]pyridin-3-yl-amine (7)

Intermediate 57 was reacted with commercially available 2-bromo-1-(3,4-dimethoxyphenyl)-ethanone according to the general procedure to afford the title compound in 80% yield; mp 251-254° C. ¹H NMR (DMSO-d₆) δ 11.25 (s, 1H, NH), 9.47 (s, 1H), 8.39-8.55 (m, 2H), 7.95 (s, 1H), 7.48-7.55 (m, 2H), 7.47 (s, 1H), 6.95-7.06 (m, 1H), 3.87 (s, 3H), 3.81 (s, 3H); LCMS (ESI) m/z 314 (MH+)

### [4-(3,4-dimethoxyphenyl)-thiazol-2-yl]pyridin-4-yl-amine (8)

Pyridin-4-yl-thiourea (Alfa Aesar) was reacted with commercially available 2-bromo-1-(3,4-dimethoxyphenyl)-ethanone according to the general procedure to afford the title compound in 56% yield; mp 205-207° C. ¹H NMR (400 MHz, DMSO-d₆) δ 10.71 (s, 1H, NH), 8.41 (d, J=6.23 Hz, 2H), 7.62-7.69 (m, 2H), 7.52 (dd, J=2.01, 8.24 Hz, 1H), 7.48 (d, J=2.01 Hz, 1H), 7.39 (s, 1H), 7.03 (d, J=8.42 Hz, 1H), 3.85 (s, 3H), 3.79 (s, 3H); LCMS (ESI) m/z 314 (MH+)

### [4-(3,4-dimethoxyphenyl)-thiazol-2-yl]-phenyl-amine (9)

N-Phenylthiourea (AK Scientific) was reacted with commercially available 2-bromo-1-(3,4-dimethoxyphenyl)-ethanone according to the general procedure to afford the title compound in 67% yield; mp 170-174° C. ¹H NMR (400 MHz, DMSO-d₆) δ 10.23 (s, 1H, NH), 7.71 (dd, J=0.92, 8.61 Hz, 2H), 7.49-7.53 (m, 1H), 7.47 (t, J=1.92 Hz, 1H), 7.30-7.39 (m, 2H), 7.22 (s, 1H), 6.99-7.05 (m, 1H), 6.93-6.99 (m, 1H), 3.82 (s, 3H), 3.78 (s, 3H); LCMS (ESI) m/z 313 (MH+)

### [4-(3,4-dimethoxyphenyl)-thiazol-2-yl]pyrimidin-2-yl-amine (10)

Intermediate 61 was reacted with commercially available 2-bromo-1-(3,4-dimethoxyphenyl)-ethanone according to the general procedure to afford the title compound in 62% yield; mp 225-228° C. ¹H NMR (DMSO-d₆) δ 11.80 (br. s., 1H, NH), 8.65 (s, 1H), 8.64 (s, 1H), 7.49 (s, 1H), 7.46 (d, J=1.28 Hz, 1H), 7.43 (s, 1H), 7.04 (td, J=0.73, 4.85 Hz, 1H), 7.00 (d, J=8.06 Hz, 1H), 3.82 (s, 3H), 3.78 (s, 3H); LCMS (ESI) m/z 315 (MH+)

### [4-(3,4-dimethoxyphenyl)-thiazol-2-yl]pyrazin-2-yl-amine (11)

Intermediate 58 was reacted with commercially available 2-bromo-1-(3,4-dimethoxyphenyl)-ethanone according to the general procedure to afford the title compound in 66% yield; mp 205-207° C. ¹H NMR (400 MHz, DMSO-d₆) δ 11.83 (s, 1H, NH), 8.50 (d, J=1.46 Hz, 1H), 8.32 (dd, J=1.46, 2.75 Hz, 1H), 8.13 (d, J=2.93 Hz, 1H), 7.47-7.51 (m, 1H), 7.46 (d, J=2.01 Hz, 1H), 7.43 (s, 1H), 7.01 (d, J=8.24 Hz, 1H), 3.83 (s, 3H), 3.79 (s, 3H); LCMS (ESI) m/z 315 (MH+)

### [4-(3,4-dimethoxyphenyl)-thiazol-2-yl]-(5-trifluoromethylpyridin-2-yl)-amine (12)

Intermediate 55 was reacted with commercially available 2-bromo-1-(3,4-dimethoxyphenyl)-ethanone according to the general procedure to afford the title compound in 68% yield; mp 244-247° C. ¹H NMR (DMSO-d₆) δ 11.90 (br. s., 1H, NH), 8.68 (s, 1H), 8.05 (dd, J=2.56, 8.79 Hz, 1H), 7.47-7.51 (m, 1H), 7.46 (d, J=2.01 Hz, 1H), 7.44 (s, 1H), 7.24 (d, J=8.79 Hz, 1H), 7.00 (d, J=8.24 Hz, 1H), 3.82 (s, 3H), 3.79 (s, 3H); LCMS (ESI) m/z 382 (MH+)

### [4-(3,4-dimethoxyphenyl)-thiazol-2-yl]-(5-methoxypyridin-2-yl)-amine (13)

Intermediate 56 was reacted with commercially available 2-bromo-1-(3,4-dimethoxyphenyl)-ethanone according to the general procedure to afford the title compound in 56% yield; mp 222-225° C. ¹FI NMR (400 MHz, DMSO-d₆) δ 11.32 (br. s., 1H, NH), 8.05 (d, J=2.93 Hz, 1H), 7.42-7.49 (m, 3H), 7.27 (s, 1H), 7.11 (d, J=8.97 Hz, 1H), 7.00 (d, J=8.42 Hz, 1H), 3.82 (s, 3H), 3.81 (s, 3H), 3.78 (s, 3H); ¹³C NMR (100 MHz, DMSO-d₆) δ 160.5, 150.8, 149.4, 149.2, 149.0, 146.9, 131.9, 128.7, 126.5, 118.7, 112.5, 112.1, 110.0, 103.8, 56.6, 56.2, 56.1; LCMS (ESI) m/z 344 (MH+)

### [4-(3,4-dimethoxyphenyl)-thiazol-2-yl]-(5-methylpyridin-2-yl)-amine (14)

Intermediate 52 was reacted with commercially available 2-bromo-1-(3,4-dimethoxyphenyl)-ethanone according to the general procedure to afford the title compound in 89% yield; mp 190-192° C. ¹H NMR (DMSO-d₆) δ 11.38 (br. s., 1H, NH), 8.15 (s, 1H), 7.60 (d, J=8.24 Hz, 1H), 7.43-7.49 (m, 2H), 7.30 (s, 1H), 7.05 (d, J=8.24 Hz, 1H), 6.99 (d, J=8.24 Hz, 1H), 3.82 (s, 3H), 3.78 (s, 3H), 2.24 (s, 3H); LCMS (ESI) m/z 328 (MH+)

### [4-(3,4-dimethoxyphenyl)-thiazol-2-yl]-(3-methylpyridin-2-yl)-amine (15)

Intermediate 54 was reacted with commercially available 2-bromo-1-(3,4-dimethoxyphenyl)-ethanone according to the general procedure to afford the title compound in 92% yield; mp 264-266° C. ¹H NMR (DMSO-d₆) δ 10.65 (bs, 1H, NH), 8.21 (d, J=5.0 Hz, 1H), 7.67 (d, J=6.8 Hz, 1H), 7.51 (s, 1H), 7.50 (dd, J=10.4, 1.5, 1H), 7.39 (s, 1H), 7.00 (s, 1H), 6.98 (s, 1H), 3.82 (s, 3H), 3.77 (s, 3H), 2.37 (s, 3H); LCMS (ESI) m/z 328 (MH+)

### [4-(3,4-dimethoxyphenyl)-thiazol-2-yl]-(4-methylpyridin-2-yl)-amine (16)

Intermediate 53 was reacted with commercially available 2-bromo-1-(3,4-dimethoxyphenyl)-ethanone according to the general procedure to afford the title compound in 68% yield; mp 211-213° C. ¹H NMR (DMSO-d₆) δ 11.46 (br. s., 1H, NH), 8.19 (d, J=5.49 Hz, 1H), 7.48 (s, 1H), 7.46 (d, J=2.01 Hz, 1H), 7.33 (s, 1H), 6.99 (d, J=8.24 Hz, 1H), 6.93 (s, 1H), 6.83 (d, J=4.94 Hz, 1H), 3.83 (s, 3H), 3.79 (s, 3H), 2.31 (s, 3H); LCMS (ESI) m/z 328 (MH+)

### [4-(3,4-dimethoxyphenyl)-thiazol-2-yl]isoquinolin-3-yl-amine (17)

Intermediate 59 was reacted with commercially available 2-bromo-1-(3,4-dimethoxyphenyl)-ethanone according to the general procedure to afford the title compound in 78% yield; mp 200-203° C. ¹H NMR (400 MHz, DMSO-d₆) δ 11.40 (s, 1H, NH), 9.19 (s, 1H), 8.03 (d, J=8.24 Hz, 1H), 7.80 (d, J=8.06 Hz, 1H), 7.65 (td, J=1.19, 7.55 Hz, 1H), 7.47-7.55 (m, 3H), 7.39-7.45 (m, 1H), 7.29 (s, 1H), 7.01 (d, J=8.24 Hz, 1H), 3.84 (s, 3H), 3.79 (s, 3H). ¹³C NMR (100 MHz, Chloroform-d) δ 161.4, 150.1, 149.6, 149.0, 148.7, 148.4, 138.3, 130.6, 128.6, 127.8, 125.7, 124.7, 124.3, 118.6, 111.4, 109.6, 103.1, 103.0, 56.0, 56.0; LCMS (ESI) m/z 364 (MH+)

### [4-(3,4-dimethoxyphenyl)-thiazol-2-yl]-quinolin-2-yl-amine (18)

Intermediate 60 was reacted with commercially available 2-bromo-1-(3,4-dimethoxyphenyl)-ethanone according to the general procedure to afford the title compound in 80% yield; mp 257-259° C. ¹H NMR (400 MHz, DMSO-d₆) δ 11.82 (br. s., 1H, NH), 8.23 (d, J=8.97 Hz, 1H), 7.83-7.88 (m, 2H), 7.69 (td, J=1.37, 7.65 Hz, 1H), 7.47-7.53 (m, 2H), 7.44 (s, 1H), 7.39-7.44 (m, 1H), 7.28 (d, J=8.79 Hz, 1H), 7.02 (d, J=8.24 Hz, 1H), 3.84 (s, 3H), 3.79 (s, 3H); ¹³C NMR (100 MHz, DMSO-d₆) δ 159.7, 151.2, 149.5, 149.4, 149.1, 146.6, 138.5, 130.6, 128.6, 128.5, 126.6, 124.7, 124.4, 118.7, 113.5, 112.6, 110.0, 105.8, 56.2, 56.2; LCMS (ESI) m/z 364 (MH+)

### [4-(3,4-dimethoxyphenyl)-thiazol-2-yl]-naphthalen-2-yl-amine (19)

Intermediate 64 was reacted with commercially available 2-bromo-1-(3,4-dimethoxyphenyl)-ethanone according to the general procedure to afford the title compound in 68% yield; mp 221-224° C. ¹H NMR (400 MHz, DMSO-d₆) δ 10.49 (s, 1H, NH), 8.54 (s, 1H), 7.87 (d, J=8.97 Hz, 1H), 7.81 (t, J=9.16 Hz, 2H), 7.53-7.61 (m, 3H), 7.47 (t, J=7.42 Hz, 1H), 7.31-7.38 (m, 1H), 7.29 (s, 1H), 7.05 (d, J=8.79 Hz, 1H), 3.89 (s, 3H), 3.81 (s, 3H); LCMS (ESI) m/z 363 (MH+)

### [4-(3,4-dimethoxyphenyl)-thiazol-2-yl]-naphthalen-1-yl-amine (20)

Naphthalen-1-yl-thiourea (TCI America) was reacted with commercially available 2-bromo-1-(3,4-dimethoxyphenyl)-ethanone according to the general procedure to afford the title compound in 59% yield; mp 244-246° C. ¹H NMR (400 MHz, DMSO-d₆) δ 10.24 (br. s., 1H, NH), 8.27-8.33 (m, 1H), 8.23 (dd, J=2.93, 7.51 Hz, 1H), 7.96 (dt, J=2.38, 4.76 Hz, 1H), 7.70 (d, J=8.06 Hz, 1H), 7.51-7.61 (m, 3H), 7.41-7.48 (m, 2H), 7.21 (s, 1H), 7.01 (d, J=8.97 Hz, 1H), 3.83 (s, 3H), 3.79 (s, 3H); LCMS (ESI) m/z 363 (MH+)

### [4-(4-Methoxyphenyl)-thiazol-2-yl]-(5-methylpyridin-2-yl)-amine (21)

Intermediate 52 was reacted with commercially available 2-bromo-1-(4-methoxyphenyl)-ethanone according to the general procedure to afford the title compound in 73% yield; mp 246-248° C. ¹H NMR (400 MHz, DMSO-d₆) δ 11.60 (br. s., 1H, NH), 8.21 (s, 1H), 7.79-7.94 (m, J=8.61 Hz, 2H), 7.69 (br. s., 1H), 7.32 (br. s., 1H), 7.13 (br. s., 1H), 6.97-7.04 (m, J=7.87 Hz, 2H), 3.81 (s, 3H), 2.28 (s, 3H); LCMS (ESI) m/z 298 (MH+)

### [4-(2-Methoxyphenyl)-thiazol-2-yl]-(5-methylpyridin-2-yl)-amine (22)

Intermediate 52 was reacted with commercially available 2-bromo-1-(2-methoxyphenyl)-ethanone according to the general procedure to afford the title compound in 78% yield; mp 228-230° C. ¹H NMR (400 MHz, methanol-d₄) δ 8.34 (s, 1H), 8.00 (dd, J=1.92, 8.70 Hz, 1H), 7.94 (d, J=7.87 Hz, 1H), 7.62 (s, 1H), 7.40-7.49 (m, 1H), 7.25 (d, J=8.79 Hz, 1H), 7.20 (d, J=8.42 Hz, 1H), 7.08-7.16 (m, 1H), 4.02 (s, 3H), 2.41 (s, 3H); LCMS (ESI) m/z 298 (MH+)

### (5-Methylpyridin-2-yl)[4-pyridin-4-yl)-thiazol-2-yl]-amine (23)

Intermediate 52 was reacted with commercially available 2-bromo-1-pyridin-4-yl-ethanone according to the general procedure to afford the title compound in 72% yield; mp decomposition at 291° C. ¹H NMR (400 MHz, DMSO-d₆) δ 11.58 (br. s., 1H, NH), 8.90-8.95 (m, 2H), 8.42-8.46 (m, 2H), 8.40 (s, 1H), 8.16-8.20 (m, 1H), 7.62 (dd, J=2.38, 8.42 Hz, 1H), 7.07 (d, J=8.24 Hz, 1H), 2.25 (s, 3H); LCMS (ESI) m/z 269 (MH+)

### (5-Methylpyridin-2-yl)-(4-pyridin-2-yl-thiazol-2-yl)-amine (24)

Intermediate 52 was reacted with commercially available 2-bromo-1-pyridin-2-yl-ethanone according to the general procedure to afford the title compound in 62% yield; mp 184-190° C. ¹H NMR (400 MHz, DMSO-d₆) δ 11.31 (s, 1H, NH), 8.59 (dt, J=0.92, 4.76 Hz, 1H), 8.13-8.17 (m, 1H), 7.93-7.98 (m, 1H), 7.87 (td, J=1.28, 7.69 Hz, 1H), 7.61 (s, 1H), 7.56 (dd, J=2.20, 8.42 Hz, 1H), 7.27-7.34 (m, 1H), 7.03 (d, J=8.42 Hz, 1H), 2.23 (s, 3H); LCMS (ESI) m/z 269 (MH+)

### (5-Methylpyridin-2-yl)-(4-phenylthiazol-2-yl)-amine (25)

Intermediate 52 was reacted with commercially available 2-bromo-1-phenyl-ethanone according to the general procedure to afford the title compound in 82% yield; mp 260-264° C. ¹H NMR (400 MHz, methanol-d₄) δ 8.32-8.37 (m, 1H), 8.17 (dd, J=2.01, 8.97 Hz, 1H), 7.93-8.01 (m, 2H), 7.54 (s, 1H), 7.45-7.52 (m, 2H), 7.33-7.45 (m, 2H), 2.43 (s, 3H); LCMS (ESI) m/z 268 (MH+)

### (5-Methylpyridin-2-yl)-[4-(5-pyridin-2-yl-thiophen-2-yl)-thiazol-2-yl]-amine (26)

Intermediate 52 was reacted with commercially available 2-bromo-1-(5-pyridin-2-yl-thiophen-2-yl)-ethanone according to the general procedure to afford the title compound in 73% yield; mp 197-199° C. ¹H NMR (400 MHz, DMSO-d₆) δ 11.37 (s, 1H, NH), 8.52 (d, J=5.86 Hz, 1H), 8.13 (s, 1H), 7.88-7.92 (m, 1H), 7.77-7.86 (m, 1H), 7.73-7.77 (m, 1H), 7.55 (dd, J=2.20, 8.42 Hz, 1H), 7.50 (d, J=3.85 Hz, 1H), 7.33 (s, 1H), 7.25 (dd, J=4.76, 7.33 Hz, 1H), 7.02 (d, J=8.42 Hz, 1H), 2.22 (s, 3H); LCMS (ESI) m/z 351 (MH+)

### (5-Methylpyridin-2-yl)-[4-(3-phenylisoxazol-5-yl)-thiazol-2-yl]-amine (27)

Intermediate 52 was reacted with commercially available 2-bromo-1-(3-phenylisoxazol-5-yl)-ethanone according to the general procedure to afford the title compound in 63% yield; mp 224-227° C. ¹H NMR (400 MHz, DMSO-d₆) δ 11.56 (s, 1H, NH), 8.17 (s, 1H), 7.88-7.99 (m, 2H), 7.65 (s, 1H), 7.51-7.61 (m, 4H), 7.19 (s, 1H), 7.02 (d, J=8.42 Hz, 1H), 2.24 (s, 3H); ¹³C NMR (100 MHz, DMSO-d₆) δ 166.9, 162.9, 161.3, 150.1, 146.4, 139.7, 137.9, 131.0, 129.8 (2), 129.2, 127.4 (2), 125.8, 112.1, 111.2, 99.0, 17.9; LCMS (ESI) m/z 335 (MH+)

### [4-(5-Methyl-3-phenylisoxazol-4-yl)-thiazol-2-yl]-(5-methylpyridin-2-yl)-amine (28)

Intermediate 52 was reacted with commercially available 2-bromo-1-(5-methyl-3-phenylisoxazol-4-yl)-ethanone according to the general procedure to afford the title compound in 54% yield; mp 267-269° C. ¹H NMR (400 MHz, methanol-d₄) δ 8.12 (dd, J=2.11, 8.88 Hz, 1H), 7.71 (s, 1H), 7.51-7.57 (m, 2H), 7.40-7.48 (m, 3H), 7.31 (d, J=8.97 Hz, 1H), 7.22 (s, 1H), 2.64 (s, 3H), 2.37 (s, 3H); LCMS (ESI) m/z 349 (MH+)

### (5-Methylpyridin-2-yl)-(4-methylthiazol-2-yl)-amine (29)

Intermediate 52 was reacted with commercially available 1-bromo-propan-2-one according to the general procedure to afford the title compound in 64% yield; mp 212-214° C. ¹H NMR (400 MHz, DMSO-d₆) δ d 11.01 (br. s., 1H, NH), 8.09 (s, 1H), 7.51 (dd, J=2.01, 8.42 Hz, 1H), 6.94 (d, J=8.42 Hz, 1H), 6.48 (s, 1H), 2.21 (s, 3H), 2.20 (s, 3H); LCMS (ESI) m/z 206 (MH+)

### Isoquinolin-3-yl-[4-(4-methoxyphenyl)-thiazol-2-yl]-amine (30)

Intermediate 59 was reacted with commercially available 2-bromo-1-(4-methoxyphenyl)-ethanone according to the general procedure to afford the title compound in 57% yield; mp 224-228° C. ¹H NMR (400 MHz, DMSO-d₆) δ 11.37 (s, 1H, NH), 9.19 (s, 1H), 8.02 (d, J=8.06 Hz, 1H), 7.84-7.92 (m, 2H), 7.81 (d, J=8.24 Hz, 1H), 7.61-7.71 (m, 1H), 7.54 (br. s., 1H), 7.38-7.47 (m, 1H), 7.19-7.28 (m, 1H), 6.93-7.05 (m, 2H), 3.79 (s, 3H); LCMS (ESI) m/z 334 (MH+)

### Isoquinolin-3-yl-[4-(4-trifluoromethoxyphenyl)-thiazol-2-yl]-amine (31)

Intermediate 59 was reacted with commercially available 2-bromo-1-(4-trifluoromethoxyphenyl)-ethanone according to the general procedure to afford the title compound in 67% yield; mp 269-271° C. ¹H NMR (400 MHz, DMSO-d₆) δ 11.46 (s, 1H), 9.20 (s, 1H), 8.00-8.09 (m, 3H), 7.83 (d, J=8.42 Hz, 1H), 7.66 (t, J=7.60 Hz, 1H), 7.54 (s, 1H), 7.50 (s, 1H), 7.37-7.47 (m, 3H); LCMS (ESI) m/z 388 (MH+)

### Isoquinolin-3-yl-[4-pyridin-4-yl)-thiazol-2-yl]-amine (32)

Intermediate 59 was reacted with commercially available 2-bromo-1-pyridin-4-yl-ethanone according to the general procedure to afford the title compound in 52% yield; mp 259-260° C. ¹H NMR (400 MHz, DMSO-d₆) δ 11.54 (s, 1H, NH), 9.22 (s, 1H), 8.58-8.70 (m, 2H), 8.05 (d, J=8.24 Hz, 1H), 7.87-7.91 (m, 2H), 7.85 (d, J=8.24 Hz, 1H), 7.80 (s, 1H), 7.66 (ddd, J=1.10, 6.91, 8.29 Hz, 1H), 7.55 (s, 1H), 7.41-7.49 (m, 1H); LCMS (ESI) m/z 305 (MH+)

### Isoquinolin-3-yl-(4-phenylthiazol-2-yl)-amine (33)

Intermediate 59 was reacted with commercially available 2-bromo-1-phenyl-ethanone according to the general procedure to afford the title compound in 72% yield; mp 265-267° C. ¹H NMR (400 MHz, DMSO-d₆) δ 11.44 (br. s., 1H, NH), 9.21 (s, 1H), 8.04 (d, J=8.42 Hz, 1H), 7.91-7.97 (m, 2H), 7.83 (d, J=8.61 Hz, 1H), 7.66 (dd, J=7.05, 8.15 Hz, 1H), 7.55 (s, 1H), 7.39-7.48 (m, 4H), 7.28-7.35 (m, 1H); LCMS (ESI) m/z 304 (MH+)

### Isoquinolin-3-yl-[4-(5-pyridin-2-yl-thiophen-2-yl)-thiazol-2-yl]-amine (34)

Intermediate 59 was reacted with commercially available 2-bromo-1-(5-pyridin-2-yl-thiophen-2-yl)-ethanone according to the general procedure to afford the title compound in 63% yield; mp 246-248° C. ¹H NMR (400 MHz, DMSO-d₆) δ 11.54 (br. s., 1H, NH), 9.20 (s, 1H), 8.52-8.57 (m, 1H), 8.03 (d, J=8.24 Hz, 1H), 7.90-7.95 (m, 1H), 7.79-7.87 (m, 2H), 7.78 (d, J=4.03 Hz, 1H), 7.66 (ddd, J=1.10, 6.91, 8.29 Hz, 1H), 7.54 (d, J=3.85 Hz, 1H), 7.49 (s, 1H), 7.43 (ddd, J=0.92, 6.91, 8.10 Hz, 1H), 7.36 (s, 1H), 7.27 (ddd, J=1.10, 4.90, 7.37 Hz, 1H); LCMS (ESI) m/z 387 (MH+)

### Isoquinolin-3-yl-[4-(3-phenylisoxazol-5-yl)-thiazol-2-yl]-amine (35)

Intermediate 59 was reacted with commercially available 2-bromo-1-(3-phenylisoxazol-5-yl)-ethanone according to the general procedure to afford the title compound in 59% yield; mp decomposition 260° C. ¹H NMR (400 MHz, DMSO-d₆) δ 11.68 (s, 1H, NH), 9.21 (s, 1H), 8.04 (d, J=8.42 Hz, 1H), 7.94 (dd, J=1.74, 6.32 Hz, 2H), 7.85 (d, J=8.24 Hz, 1H), 7.62-7.70 (m, 2H), 7.48-7.58 (m, 4H), 7.39-7.48 (m, 1H), 7.24 (s, 1H); LCMS (ESI) m/z 371 (MH+)

### Isoquinolin-3-yl-(4-methylthiazol-2-yl)-amine (36)

Intermediate 59 was reacted with commercially available 1-bromo-propan-2-one according to the general procedure to afford the title compound in 73% yield; mp 212-215° C. ¹H NMR (400 MHz, DMSO-d₆) δ 11.16 (br. s., 1H, NH), 9.15 (s, 1H), 8.00 (d, J=7.87 Hz, 1H), 7.77 (d, J=8.24 Hz, 1H), 7.57-7.70 (m, 1H), 7.47 (s, 1H), 7.34-7.45 (m, 1H), 6.51 (s, 1H), 2.26 (s, 3H); LCMS (ESI) m/z 242 (MH+)

### Isoquinolin-3-yl-(4-trifluoromethylthiazol-2-yl)-amine (37)

Intermediate 59 was reacted with commercially available 3-bromo-1,1,1-trifluoro-propan-2-one according to the general procedure to afford the title compound in 67% yield; mp 200-203° C. ¹H NMR (400 MHz, DMSO-d₆) δ 11.81 (s, 1H), 9.23 (s, 1H), 8.02-8.09 (m, 1H), 7.80-7.87 (m, 1H), 7.64-7.72 (m, 2H), 7.46 (ddd, J=1.01, 6.96, 8.15 Hz, 1H), 7.37 (s, 1H); LCMS (ESI) m/z 296 (MH+)

### (8H-Indeno[1,2-d]thiazol-2-yl)-(5-methylpyridin-2-yl)-amine (38)

A solution of intermediate 52 (1 mmol) and commercially available 2-bromoindan-1-one (1 mmol, 1 eq.) in EtOH (10 mL) was heated to 60° C. for 3 h, after which time the reaction was judged complete. The reaction mixture was then poured into water-ice (20 mL) and stirred for 30 minutes. Aqueous 1N Na₂CO₃ was then added to this solution until a pH 8 was reached. The product precipitated from this solution and was collected by filtration and washed with water. The crude product was purified by column chromatography (40-80% ethyl acetate-hexane). Relevant fractions were collected and evaporated to afford the product in 69% yield; mp decomposition 282° C. ¹H NMR (400 MHz, DMSO-d₆) δ 11.85 (s, 1H), 8.18-8.23 (m, 1H), 7.74 (d, J=8.24 Hz, 1H), 7.63 (d, J=7.33 Hz, 1H), 7.54 (d, J=7.51 Hz, 1H), 7.37 (t, J=7.42 Hz, 1H), 7.20-7.27 (m, 1H), 7.13 (d, J=8.42 Hz, 1H), 3.85 (s, 2H), 2.28 (s, 3H); LCMS (ESI) m/z 280 (MH+)

### (5,6-Dimethoxy-8H-indeno-[1,2-d]thiazol-2-yl)-(5-methylpyridin-2-yl)-amine (39)

A solution of intermediate 52 (1 mmol) and bromo-5,6-dimethoxy-indan-1-one (65, 1 mmol, 1 eq.) in EtOH (10 mL) was heated to 60° C. for 3 h, after which time the reaction was judged complete. The reaction mixture was then poured into water-ice (20 mL) and stirred for 30 minutes. Aqueous 1N Na₂CO₃ was then added to this solution until a pH 8 was reached. The product precipitated from this solution and was collected by filtration and washed with water. The crude product was purified by column chromatography (40-80% ethyl acetate-hexane). Relevant fractions were collected and evaporated to afford the product in 72% yield; mp decomposition 265° C. ¹H NMR (400 MHz, DMSO-d₆) δ 11.60 (s, 1H), 8.18 (s, 1H), 7.68 (d, J=8.06 Hz, 1H), 7.23 (s, 1H), 7.18 (s, 1H), 7.10 (d, J=8.79 Hz, 1H), 3.83 (s, 3H), 3.79 (s, 3H), 3.74 (s, 2H), 2.26 (s, 3H); LCMS (ESI) m/z 340 (MH+)

### (5-Methoxypyridin-2-yl)-[4-(3-phenylisoxazol-5-yl)-thiazol-2-yl]-amine (40)

Intermediate 56 was reacted with commercially available 2-bromo-1-(3-phenylisoxazol-5-yl)-ethanone according to the general procedure to afford the title compound in 53% yield; mp 203-205° C. ¹H NMR (400 MHz, DMSO-d₆) δ 11.49 (s, 1H, NH), 8.06 (d, J=3.11 Hz, 1H), 7.88-7.97 (m, 2H), 7.62 (s, 1H), 7.50-7.59 (m, 3H), 7.47 (dd, J=2.93, 8.97 Hz, 1H), 7.19 (s, 1H), 7.09 (d, J=8.97 Hz, 1H), 3.81 (s, 3H); LCMS (ESI) m/z 351 (MH+)

### (4-Methoxypyridin-2-yl)-[4-(3-phenylisoxazol-5-yl)-thiazol-2-yl]-amine (41)

Intermediate 63 was reacted with commercially available 2-bromo-1-(3-phenylisoxazol-5-yl)-ethanone according to the general procedure to afford the title compound in 58% yield; mp 177-179° C. ¹H NMR (400 MHz, DMSO-d₆) δ 11.54 (s, 1H), 8.16 (d, J=6.23 Hz, 1H), 7.88-8.01 (m, 2H), 7.67 (s, 1H), 7.47-7.59 (m, 3H), 7.20 (s, 1H), 6.57-6.66 (m, 2H), 3.82 (s, 3H); LCMS (ESI) m/z 351 (MH+)

### [4-(3-Phenylisoxazol-5-yl)-thiazol-2-yl]pyridin-2-yl-amine (42)

Intermediate 62 was reacted with commercially available 2-bromo-1-(3-phenylisoxazol-5-yl)-ethanone according to the general procedure to afford the title compound in 52% yield; mp 211-215° C. ¹H NMR (400 MHz, DMSO-d₆) δ 11.68 (s, 1H, NH), 8.34 (dd, J=0.92, 5.13 Hz, 1H), 7.90-7.99 (m, 2H), 7.75 (ddd, J=1.83, 7.05, 8.52 Hz, 1H), 7.69 (s, 1H), 7.51-7.59 (m, 3H), 7.21 (s, 1H), 7.10 (d, J=8.24 Hz, 1H), 6.98 (ddd, J=0.92, 5.63, 6.64 Hz, 1H); LCMS (ESI) m/z 321 (MH+)

### (4-Methylpyridin-2-yl)-[4-(3-phenylisoxazol-5-yl)-thiazol-2-yl]-amine (43)

Intermediate 53 was reacted with commercially available 2-bromo-1-(3-phenylisoxazol-5-yl)-ethanone according to the general procedure to afford the title compound in 62% yield; mp decomposition 296° C. ¹H NMR (400 MHz, DMSO-d₆) δ 11.70 (br. s., 1H, NH), 8.22 (d, J=5.31 Hz, 1H), 7.88-7.98 (m, 2H), 7.70 (s, 1H), 7.49-7.60 (m, 3H), 7.25 (s, 1H), 6.93 (s, 1H), 6.86 (d, J=5.31 Hz, 1H), 2.32 (s, 3H); LCMS (ESI) m/z 335 (MH+)

### (6-Methylpyridin-2-yl)-[4-(3-phenylisoxazol-5-yl)-thiazol-2-yl]-amine (44)

Intermediate 51 was reacted with commercially available 2-bromo-1-(3-phenylisoxazol-5-yl)-ethanone according to the general procedure to afford the title compound in 58% yield; mp 291-293° C. ¹H NMR (400 MHz, methanol-d₄) δ 8.17-8.26 (m, 1H), 7.86-7.95 (m, 3H), 7.48-7.55 (m, 3H), 7.26-7.34 (m, 3H), 2.85 (s, 3H); LCMS (ESI) m/z 335 (MH+)

### [4-(3-Phenylisoxazol-5-yl)-thiazol-2-yl]-pyridin-4-yl-amine (45)

Pyridin-4-yl-thiourea (Alfa Aesar) was reacted with commercially available 2-bromo-1-(3-phenylisoxazol-5-yl)-ethanone according to the general procedure to afford the title compound in 57% yield; mp 269-270° C. ¹H NMR (400 MHz, DMSO-d₆) δ 10.92 (br. s., 1H), 8.39-8.46 (m, 2H), 7.95-8.04 (m, 2H), 7.66-7.73 (m, 3H), 7.51-7.61 (m, 3H), 7.45 (s, 1H); LCMS (ESI) m/z 321 (MH+)

### 2-(5-Methylpyridin-2-ylamino)-thiazole-4-carboxylic acid (3,4-dimethoxyphenyl)-amide (46)

A solution of commercially available 3,4-dimethoxyphenylamine (0.13 mmol, 1 eq.) and triethylamine (0.65 mmol, 5 eq.) in THF (2 mL) and was added to a solution of 67 (0.13 mmol) and HATU (0.13 mmol) in THF (2 mL). The reaction mixture was stirred for 3 h at 60° C. After cooling, the mixture was concentrated and the crude product taken into 100 mL ethyl acetate and washed with water. The organic phase was dried (MgSO₄), filtered, and concentrated. The crude residue was purified by column chromatography on silica gel (30-60% ethyl acetate-hexane) and relevant fractions collected and concentrated to afford the desired product as a white powder in 76% yield; mp 212-214° C. ¹H NMR (400 MHz, DMSO-d₆) δ 11.36 (s, 1H), 9.55 (s, 1H), 8.15-8.17 (m, 1H), 7.69 (s, 1H), 7.58 (dd, J=2.20, 8.61 Hz, 1H), 7.45 (d, J=2.38 Hz, 1H), 7.30 (dd, J=2.38, 8.61 Hz, 1H), 7.05 (d, J=8.42 Hz, 1H), 6.93 (d, J=8.79 Hz, 1H), 3.76 (s, 3H), 3.74 (s, 3H), 2.24 (s, 3H); LCMS (ESI) m/z 371 (MH+)

### Isoquinoline-3-carboxylic acid [4-(3,4-dimethoxyphenyl)-thiazol-2-yl]amide (47)

A solution of 70 (30 mg, 0.13 mmol, 1 eq) and triethylamine (0.65 mmol, 5 eq.) in THF (2 mL) was added to a solution of commercially available isoquinoline-3-carboxylic acid (22 mg, 0.13 mmol) and HATU (50 mg, 0.13 mmol) in THF (2 mL). The reaction was stirred for 3 h at 60° C., then cooled to room temperature and poured into water-ice. The product was extracted with EtOAc, dried (MgSO₄), filtered and concentrated. The crude product was purified by column chromatography on silica gel (ethyl acetate-hexanes) to afford the product as a white powder in 39% yield; mp 228-230° C. ¹H NMR (400 MHz, DMSO-d₆) δ 11.99 (s, 2H), 9.51 (s, 1H), 8.77 (s, 1H), 8.34 (d, J=7.33 Hz, 1H), 8.29 (d, J=8.06 Hz, 1H), 7.87-7.99 (m, 2H), 7.66 (s, 1H), 7.56 (d, J=2.01 Hz, 1H), 7.53 (dd, J=1.92, 8.33 Hz, 1H), 7.03 (d, J=8.42 Hz, 1H), 3.85 (s, 3H), 3.80 (s, 3H); LCMS (ESI) m/z 392 (MH+)

### 4-(3,4-dimethoxyphenyl)-thiazole-2-carboxylic acid isoquinolin-3-ylamide (48)

A solution of commercially available isoquinolin-3-ylamine (0.06 g, 0.19 mmol, 1 eq) and triethylamine (0.95 mmol, 5 eq.) in THF (3 mL) was added to a mixture of 69 (0.10 g, 0.19 mmol) and HATU (0.15 g, 0.19 mmol) in THF (3 mL). The reaction mixture was stirred for 3 h at 60° C., then cooled to room temperature and poured into water-ice. This solution was extracted with EtOAc, and the organic phase dried (MgSO₄), filtered and concentrated. The crude product was purified by column chromatography on silica gel (ethyl acetate-hexanes) to afford the product as a white powder in 55% yield; mp 196-202° C. ¹H NMR (400 MHz, DMSO-d₆) δ 10.54 (s, 1H), 9.27 (s, 1H), 8.59 (s, 1H), 8.46 (s, 1H), 8.14 (d, J=8.06 Hz, 1H), 8.01 (d, J=8.24 Hz, 1H), 7.78 (ddd, J=1.19, 6.91, 8.20 Hz, 1H), 7.68-7.75 (m, 2H), 7.56-7.66 (m, 1H), 7.07 (d, J=8.24 Hz, 1H), 3.90 (s, 3H), 3.83 (s, 3H); LCMS (ESI) m/z 392 (MH+)

### [4-(3,4-dimethoxyphenyl)-thiazol-2-yl]-isoquinolin-3-yl-methyl-amine (49)

To a solution of 17 (50 mg, 0.138 mmol) in THF (3 mL) was added NaH (60% in mineral oil; 8 mg, 0.21 mmol, 1.5 eq) at 0° C. After stirring for 15 min at 0° C., the reaction mixture was treated with methyl iodide (39 mg, 17.2 μL, 0.28 mmol, 2 eq) and stirred for 2 h at room temperature. The reaction was quenched by the addition of aqueous NH₄Cl and the product was extracted with ether (2×50 mL). Combined organic phases were dried (MgSO₄), filtered and concentrated. The crude product was purified by column chromatography (10-50% ethyl acetate-hexane) to provide the product in 77% yield; mp 165-166° C. ¹H NMR (400 MHz, DMSO-d₆) δ 9.27 (s, 1H), 8.09 (d, J=8.06 Hz, 1H), 7.94 (d, J=8.42 Hz, 1H), 7.69-7.78 (m, 1H), 7.65 (s, 1H), 7.43-7.58 (m, 3H), 7.35 (s, 1H), 7.00 (d, J=8.97 Hz, 1H), 3.94 (s, 3H), 3.84 (s, 3H), 3.78 (s, 3H); ¹³C NMR (100 MHz, Chloroform-d) δ 162.9, 150.4, 149.5, 149.2, 148.9, 148.9, 138.5, 131.0, 128.9, 127.9, 126.3, 125.0, 125.0, 118.7, 111.5, 109.7, 105.0, 102.8, 56.2, 56.1, 36.3; LCMS (ESI) m/z 378 (MH+)

### N-[4-(3,4-dimethoxyphenyl)-thiazol-2-yl]-N-isoquinolin-3-yl-acetamide (50)

Acetic anhydride (5 mL) was added to 17 (50 mg, 0.138 mmol) and the reaction mixture heated to 100° C. for 4 hrs. The solution was allowed to cool to room temperature and then poured into water-ice. The product was extracted with EtOAc and the organic phase dried (MgSO₄), filtered and concentrated. The crude product was purified by column chromatography (30-60% ethyl acetate-hexane) to provide the product as a white powder in 81% yield; mp 164-165° C. ¹H NMR (400 MHz, DMSO-d₆) δ 9.46 (s, 1H), 8.32 (d, J=8.24 Hz, 1H), 8.25 (s, 1H), 8.13 (d, J=7.69 Hz, 1H), 7.88-7.95 (m, 1H), 7.84 (ddd, J=1.19, 6.91, 8.19 Hz, 1H), 7.64 (s, 1H), 7.17 (d, J=2.01 Hz, 1H), 7.02 (dd, J=1.92, 8.33 Hz, 1H), 6.82 (d, J=8.61 Hz, 1H), 3.67 (s, 3H), 3.59 (s, 3H), 2.15 (s, 3H); ¹³C NMR (100 MHz, Chloroform-d) δ 170.0, 160.1, 153.2, 149.4, 149.0, 148.9, 147.8, 137.6, 131.4, 128.7, 128.6, 128.0, 127.9, 127.2, 121.0, 118.7, 111.3, 109.6, 107.4, 56.1, 55.7, 24.0; LCMS (ESI) m/z 406 (MH+)

### (6-Methylpyridin-2-yl)-thiourea (51)

Commercially available 6-methylpyridin-2-ylamine was reacted according to the general procedure to afford the product in 58% yield; mp 188-190° C. ¹H NMR (DMSO-d₆) δ10.59 (s, 1H), 10.38 (s, 1H), 8.78 (s, 1H), 7.59 (t, J=8.0 Hz, 1H), 6.90 (d, J=8.3 Hz, 1H), 6.84 (d, J=7.6 Hz, 1H), 2.35 (s, 3H); LCMS (ESI) m/z 168 (MH+)

### (5-Methylpyridin-2-yl)-thiourea (52)

Commercially available 5-methylpyridin-2-ylamine was reacted according to the general procedure to afford the product in 60% yield; mp 178-181° C. ¹H NMR (DMSO-d₆) δ10.46 (s, 1H), 10.38 (s, 1H), 8.73 (s, 1H), 8.00 (d, J=2.2 Hz, 1H), 7.54 (dd, J=8.45, 2.3 Hz, 1H), 7.02 (d, J=8.6 Hz, 1H), 2.17 (s, 3H); LCMS (ESI) m/z 168 (MH+)

### (4-Methylpyridin-2-yl)-thiourea (53)

Commercially available 4-methylpyridin-2-ylamine was reacted according to the general procedure to afford the product in 60% yield; mp 210-212° C. ¹H NMR (DMSO-d₆) δ 10.57 (s, 1H), 10.38 (s, 1H), 8.77 (s, 1H), 8.03 (d, J=5.3 Hz, 1H), 6.91 (s, 1H), 6.83 (d, J=4.75 Hz, 1H), 2.21 (s, 3H); LCMS (ESI) m/z 168 (MH+)

### (3-Methylpyridin-2-yl)-thiourea (54)

Commercially available 3-methylpyridin-2-ylamine was reacted according to the general procedure to afford the product in 52% yield; mp 152-154° C. ¹H NMR (DMSO-d₆) δ 10.33 (s, 1H), 8.87 (s, 1H), 8.73 (s, 1H), 8.08 (d, J=4.9 Hz, 1H), 7.60 (d, J=7.6 Hz, 1H), 7.00 (dd, J=7.5, 5.0 Hz, 1H), 2.25 (s, 3H); LCMS (ESI) m/z 168 (MH+)

### (5-Trifluoromethylpyridin-2-yl)-thiourea (55)

Commercially available 5-trifluoromethylpyridin-2-ylamine was reacted according to the general procedure to afford the product in 62% yield; mp 203-205° C. ¹H NMR (DMSO-d₆) δ 10.88 (s, 1H), 10.36 (s, 1H), 9.16 (s, 1H), 8.60 (s, 1H), 8.11 (dd, J=9.0, 2.4 Hz, 1H), 7.31 (d, J=8.8 Hz, 1H); LCMS (ESI) m/z 222 (MH+)

### (5-Methoxypyridin-2-yl)-thiourea (56)

Commercially available 5-methoxypyridin-2-ylamine was reacted according to the general procedure to afford the product in 58% yield; mp 131-135° C. ¹H NMR (DMSO-d₆) δ 10.40 (s, 1H), 10.29 (s, 1H), 8.68 (s, 1H), 7.94-7.96 (m, 1H), 7.46 (dd, J=3.11, 9.16 Hz, 1H), 7.15 (d, J=8.97 Hz, 1H), 3.79 (s, 3H); LCMS (ESI) m/z 184 (MH+)

### Pyridin-3-yl-thiourea (57)

Commercially available pyridin-3-ylamine was reacted according to the general procedure to afford the product in 64% yield; mp 165-167° C. ¹H NMR (DMSO-d₆) δ 9.77 (s, 1H), 8.55 (d, J=2.56 Hz, 1H), 8.30 (dd, J=1.46, 4.76 Hz, 1H), 7.91-7.99 (m, 2H), 7.31-7.39 (m, 2H); LCMS (ESI) m/z 154 (MH+)

### Pyrazin-2-yl-thiourea (58)

Commercially available pyrazin-2-ylamine was reacted according to the general procedure to afford the product in 64% yield; mp 238-240° C. ¹H NMR (DMSO-d₆) δ10.79 (s, 1H), 9.90 (s, 1H), 9.04 (s, 1H), 8.49 (s, 1H), 8.19 (d, J=0.7 Hz, 2H); LCMS (ESI) m/z 155 (MH+)

### Isoquinolin-3-yl-thiourea (59)

Commercially available isoquinolin-3-ylamine was reacted according to the general procedure to afford the product in 74% yield; mp 225-227° C. ¹H NMR (DMSO-d₆) δ 10.60 (s, 1H), 10.25 (s, 1H), 9.13 (s, 1H), 8.72 (s, 1H), 8.07 (d, J=8.24 Hz, 1H), 7.81 (d, J=8.42 Hz, 1H), 7.70 (ddd, J=1.28, 6.87, 8.33 Hz, 1H), 7.56 (s, 1H), 7.52 (ddd, J=1.10, 6.96, 8.24 Hz, 1H); LCMS (ESI) m/z 204 (MH+)

### Quinolin-2-yl-thiourea (60)

Commercially available quinolin-2-ylamine was reacted according to the general procedure to afford the product in 68% yield; mp 179-180° C. ¹H NMR (DMSO-d₆) δ 11.10 (s, 1H), 10.80 (s, 1H), 9.17 (s, 1H), 8.29 (d, J=8.97 Hz, 1H), 7.85 (dt, J=1.56, 8.06 Hz, 2H), 7.64-7.73 (m, 1H), 7.44-7.52 (m, 1H), 7.34 (d, J=8.79 Hz, 1H); LCMS (ESI) m/z 204 (MH+)

### Pyrimidin-2-yl-thiourea (61)

Commercially available pyrimidin-2-ylamine was reacted according to the general procedure to afford the product in 77% yield; mp 262-264° C. ¹H NMR (DMSO-d₆) δ 10.53 (s, 1H), 10.16 (s, 1H), 9.09 (s, 1H), 8.60 (d, J=5.0 Hz, 2H), 7.11 (t, J=5.0 Hz, 1H); LCMS (ESI) m/z 155 (MH+)

### Pyridin-2-yl-thiourea (62)

Commercially available pyridin-2-ylamine was reacted according to the general procedure to afford the product in 67% yield; mp 144-146° C. ¹H NMR (DMSO-d₆) δ 10.57 (br. s., 1H), 10.52 (s, 1H), 8.87 (br. s., 1H), 8.23 (dd, J=1.28, 5.13 Hz, 1H), 7.69-7.82 (m, 1H), 7.16 (d, J=8.42 Hz, 1H), 7.04 (ddd, J=0.73, 5.17, 7.28 Hz, 1H); LCMS (ESI) m/z 154 (MH+)

### (4-Methoxypyridin-2-yl)-thiourea (63)

Commercially available 4-methoxypyridin-2-ylamine was reacted according to the general procedure to afford the product in 52% yield; mp 214-217° C. ¹H NMR (DMSO-d₆) δ 10.66 (br. s., 1H), 10.34 (s, 1H), 8.83 (br. s., 1H), 8.06 (d, J=6.04 Hz, 1H), 6.75 (d, J=2.20 Hz, 1H), 6.67 (dd, J=2.38, 6.04 Hz, 1H), 3.78 (s, 3H); LCMS (ESI) m/z 184 (MH+)

### Naphthalen-2-yl-thiourea (64)

Commercially available naphthalen-2-ylamine was reacted according to the general procedure to afford the product in 65% yield; mp 190-192° C. ¹H NMR (DMSO-d₆) δ 9.88 (s, 1H), 7.95 (s, 1H), 7.82-7.89 (m, 3H), 7.38-7.56 (m, 4H); LCMS (ESI) m/z 203 (MH+)

### Bromo-5,6-dimethoxy-indan-1-one (65)

A solution of bromine (1.0 g, 330 μL, 6.30 mmol, 1.2 eq.) in Et₂O (5 mL) was added dropwise to a solution of commercially available 5,6-dimethoxy-indan-1-one (1.0 g, 5.2 mmol) in Et₂O (30 mL). The reaction mixture was stirred at room temperature overnight, solvent evaporated, and the resulting crude product re-crystallized from MeOH (10 mL) to afford 0.65 g (46%) of the desired intermediate 65 a yellow solid; mp 162-163° C. ¹H NMR (400 MHz, DMSO-d₆) δ 7.15 (s, 1H), 7.13 (s, 1H), 4.96 (ddd, J=0.92, 2.75, 7.14 Hz, 1H), 3.88-3.90 (m, 3H), 3.81-3.83 (m, 3H), 3.75-3.81 (m, 1H), 3.21 (dd, J=2.75, 17.95 Hz, 1H); LCMS (ESI) m/z 272 (MH+)

### 2-(5-Methylpyridin-2-ylamino)-thiazole-4-carboxylic acid ethyl ester (66)

A solution of ethyl bromopyruvate (0.5 g, 0.32 mL, 2.6 mmol) and intermediate 52 (2.6 mmol, 1 eq.) in EtOH (10 mL) were stirred at 60° C. for 1 hour. The solvent was then evaporated to afford crude 66, which was used directly in the next step; mp 198-200° C. ¹H NMR (400 MHz, DMSO-d₆) δ 8.18 (s, 1H), 7.92 (s, 1H), 7.56 (d, J=8.25 Hz, 1H), 7.17 (d, J=8.25 Hz, 1H), 4.33 (q, J=6.95 Hz, 2H), 2.24 (s, 3H), 1.37 (t, J=6.95 Hz, 3H); LCMS (ESI) m/z 264 (MH+)

### 2-(5-Methylpyridin-2-ylamino)-thiazole-4-carboxylic acid (67)

Crude 66 (0.7 g, 2.66 mmol) and aqueous 5N HCl (3 mL) were heated in a sealed tube in a CEM microwave for 10 min at 130° C. After cooling, the precipitate was filtered and washed with water and acetone to afford 67 as a white powder in 83% yield. This material was used in the next step without further purification; mp decomposition 280° C. ¹H NMR (400 MHz, DMSO-d₆) δ 8.16 (s, 1H), 7.78-7.81 (m, 1H), 7.61-7.68 (m, 1H), 7.02-7.08 (m, 1H), 2.24 (s, 3H); LCMS (ESI) m/z 236 (MH+)

### 4-(3,4-dimethoxyphenyl)-thiazole-2-carboxylic acid ethyl ester (68)

A solution of 2-bromo-1-(3,4-dimethoxyphenyl)-ethanone (0.5 g, 0.96 mmol) and ethyl thiooxamate (0.25 g, 0.96 mmol) in EtOH (3 mL) was stirred at room temperature overnight. The solvent was then evaporated and the crude product purified by column chromatography on silica (ethyl acetate-hexanes) to afford the product as an orange powder in 97% yield; mp 100-102° C. ¹H NMR (400 MHz, DMSO-d₆) δ 8.43 (s, 1H), 7.52-7.58 (m, 2H), 7.05 (d, J=8.42 Hz, 1H), 4.41 (q, J=7.14 Hz, 2H), 3.84 (s, 3H), 3.80 (s, 3H), 1.35 (t, J=7.14 Hz, 3H); LCMS (ESI) m/z 294 (MH+)

### 4-(3,4-dimethoxyphenyl)-thiazole-2-carboxylic acid (69)

To a solution of 68 (0.55 g, 1.87 mmol) in MeOH, was added 5N NaOH (1 mL). The reaction was stirred at room temperature overnight, then the mixture was poured into water-ice and 1N HCl added to until the solution reached pH 2. This solution was extracted with EtOAc, and the organic phase dried (MgSO₄), filtered and concentrated to afford the desired product as an orange solid in 90% yield. This material could be used in the next step without further purification; mp 105-106° C. ¹H NMR (400 MHz, DMSO-d₆) δ 8.38 (s, 1H), 7.57-7.59 (m, 1H), 7.56 (s, 1H), 7.06 (d, J=8.42 Hz, 1H), 3.86 (s, 3H), 3.81 (s, 3H); LCMS (ESI) m/z 266 (MH+)

### 4-(3,4-dimethoxyphenyl)-thiazol-2-ylamine (70)

A solution of 2-bromo-1-(3,4-dimethoxyphenyl)-ethanone (0.10 g, 0.39 mmol) and thiourea (0.03 g, 0.39 mmol) in EtOH was stirred at 80° C. overnight. The solvent was evaporated and the residue was partitioned between EtOAc and sat. NaHCO₃. The organic layer was washed with water, dried (MgSO₄), filtered and concentrated. The solid residue was crystallized from ethyl acetate-hexanes to afford the product as a light yellow powder in 60% yield; mp 198-201° C. ¹H NMR (400 MHz, DMSO-d₆) δ 7.30-7.38 (m, 2H), 7.00 (s, 2H), 6.93 (d, J=8.42 Hz, 1H), 6.87 (s, 1H), 3.78 (s, 3H), 3.76 (s, 3H); LCMS (ESI) m/z 237 (MH+)

### (5-Methyl-pyridin-2-yl)-[4-(4-trifluoromethoxy-phenyl)-thiazol-2-yl]-amine (IND26)

(5-Methyl-pyridin-2-yl)-thiourea was reacted with commercially available 2-bromo-1-(4-trifluoromethoxy-phenyl)-ethanone, according to the general procedure to afford the title compound in 56% yield; mp 205-207° C. 1H NMR (400 MHz, DMSO-d6) δ 11.32 (s, 1H), 8.14 (s, 1H), 8.01 (d, J=8.42 Hz, 2H), 7.56 (dd, J=2.20, 8.42 Hz, 1H), 7.47 (s, 1H), 7.41 (d, J=8.79 Hz, 2H), 7.02 (d, J=8.24 Hz, 1H), 2.23 (s, 3H). LCMS (ESI) m/z 352 (MH+)

### 3-[2-(5-Methyl-pyridin-2-ylamino)-thiazol-4-yl]-benzonitrile (IND38)

(5-Methyl-pyridin-2-yl)-thiourea was reacted with commercially available 3-(2-bromo-acetyl)-benzonitrile, according to the general procedure to afford the title compound in 68% yield; mp 268-272° C. 1H NMR (400 MHz, methanol-d4) δ 8.46 (t, J=1.37 Hz, 1H), 8.35 (dt, J=0.94, 1.97 Hz, 1H), 8.33 (dq, J=1.03, 7.90 Hz, 1H), 8.22 (dd, J=2.20, 8.97 Hz, 1H), 7.73-7.79 (m, 2H), 7.64-7.71 (m, 1H), 7.40 (d, J=8.97 Hz, 1H), 2.46 (s, 3H). LCMS (ESI) m/z 293 (MH+)

### 2-[2-(4-Methyl-pyridin-2-ylamino)-thiazol-4-yl]-phenol (IND48)

(4-Methyl-pyridin-2-yl)-thiourea was reacted with commercially available 2-bromo-1-(2-hydroxy-phenyl)-ethanone, according to the general procedure to afford the title compound in 70% yield; mp 249-250° C. 1H NMR (400 MHz, DMSO-d6) δ 11.74 (br. s., 1H), 8.25 (d, J=5.13 Hz, 1H), 7.88 (d, J=7.69 Hz, 1H), 7.56 (s, 1H), 7.12-7.24 (m, 1H), 6.83-7.01 (m, 4H), 2.35 (s, 3H). LCMS (ESI) m/z 284 (MH+)

### [4-(2,3-Dihydro-benzo[1,4]dioxin-6-yl)-thiazol-2-yl]-(4-methyl-pyridin-2-yl)-amine (IND57)

4-Methyl-pyridin-2-yl)-thiourea was reacted with commercially available 2-bromo-1-(2,3-dihydro-benzo[1,4]dioxin-6-yl)-ethanone, according to the general procedure to afford the title compound in 40% yield; mp 230-240° C. 1H NMR (400 MHz, DMSO-d6) δ 11.64 (br. s., 1H), 8.19 (d, J=5.49 Hz, 1H), 7.34-7.43 (m, 2H), 7.31 (s, 1H), 6.95 (s, 1H), 6.85 (d, J=8.24 Hz, 2H), 4.23 (s, 4H), 2.30 (s, 3H). LCMS (ESI) m/z 326 (MH+)

### {4-[3-(4-Chloro-phenyl)-isoxazol-5-yl]-thiazol-2-yl}-(4-methyl-pyridin-2-yl)-amine (IND64)

(4-Methyl-pyridin-2-yl)-thiourea was reacted with commercially available 2-bromo-1-[3-(4-chloro-phenyl)-isoxazol-5-yl]-ethanone, according to the general procedure to afford the title compound in 57% yield; mp 209-211° C. 1H NMR (400 MHz, DMSO-d6) δ 11.81 (br. s., 1H), 8.25 (d, J=5.31 Hz, 1H), 7.96 (dd, J=2.01, 8.61 Hz, 2H), 7.72 (s, 1H), 7.61 (dd, J=2.20, 8.42 Hz, 3H), 7.33 (br. s., 1H), 6.96 (s, 1H), 6.90 (d, J=4.58 Hz, 1H), 2.34 (s, 3H). LCMS (ESI) m/z 369 (MH+)

### [4-(2,4-Dimethoxy-phenyl)-thiazol-2-yl]-(5-methyl-pyridin-2-yl)-amine (IND74)

(5-Methyl-pyridin-2-yl)-thiourea was reacted with commercially available 2-bromo-1-(2,4-dimethoxy-phenyl)-ethanone, according to the general procedure to afford the title compound in 78% yield; mp 157-158° C. 1H NMR (400 MHz, DMSO-d6) δ 11.75 (br. s., 1H), 8.22 (s, 1H), 7.97 (d, J=8.61 Hz, 1H), 7.72 (d, J=8.06 Hz, 1H), 7.38 (s, 1H), 7.16 (d, J=8.42 Hz, 1H), 6.69 (s, 1H), 6.64 (dt, J=1.21, 7.46 Hz, 1H), 3.92 (s, 3H), 3.82 (d, J=1.10 Hz, 3H), 2.27 (s, 3H). LCMS (ESI) m/z 328 (MH+)

### (4-Methyl-pyridin-2-yl)-[4-(4-trifluoromethoxy-phenyl)-thiazol-2-yl]-amine (IND78)

(4-Methyl-pyridin-2-yl)-thiourea was reacted with commercially available 2-bromo-1-(4-trifluoromethoxy-phenyl)-ethanone, according to the general procedure to afford the title compound in 58% yield; mp 275-282° C. 1H NMR (400 MHz, DMSO-d6) δ 11.50 (br. s., 1H), 8.20 (d, J=5.31 Hz, 1H), 8.01-8.06 (m, 2H), 7.53 (s, 1H), 7.33-7.46 (m, J=8.97 Hz, 2H), 6.93 (s, 1H), 6.84 (d, J=5.49 Hz, 1H), 2.31 (s, 3H). LCMS (ESI) m/z 352 (MH+)

### (4-Benzofuran-2-yl-thiazol-2-yl)-isoquinolin-3-yl-amine (IND91)

Isoquinolin-3-yl-thiourea was reacted with commercially available 1-benzofuran-2-yl-2-bromo-ethanone, according to the general procedure to afford the title compound in 86% yield; mp 248-250° C. 1H NMR (400 MHz, DMSO-d6) δ 11.62 (s, 1H), 9.20 (s, 1H), 8.03 (d, J=8.42 Hz, 1H), 7.84 (d, J=8.61 Hz, 1H), 7.62-7.71 (m, 2H), 7.60 (d, J=7.87 Hz, 1H), 7.50 (s, 1H), 7.38-7.47 (m, 2H), 7.22-7.36 (m, 2H), 7.14 (s, 1H). LCMS (ESI) m/z 344 (MH+)

### (4-Methoxy-pyridin-2-yl)-(4-pyridin-4-yl-thiazol-2-yl)-amine (IND111)

(4-Methoxy-pyridin-2-yl)-thiourea was reacted with commercially available 2-bromo-1-pyridin-4-yl-ethanone, according to the general procedure to afford the title compound in 88% yield; mp 190-192° C. 1H NMR (400 MHz, DMSO-d6) δ 11.38 (br. s., 1H), 8.60 (d, J=5.68 Hz, 2H), 8.14 (d, J=5.86 Hz, 1H), 7.83 (d, J=5.68 Hz, 2H), 7.77 (s, 1H), 6.63 (s, 1H), 6.56-6.61 (m, 1H), 3.82 (s, 3H). LCMS (ESI) m/z 285 (MH+)

(5-Methoxy-pyridin-2-yl)-(4-pyridin-4-yl-thiazol-2-yl)-amine (IND119)

(5-M ethoxy-pyridin-2-yl)-thiourea was reacted with commercially available 2-bromo-1-pyridin-4-yl-ethanone, according to the general procedure to afford the title compound in 64% yield; mp 213-214° C. 1H NMR (400 MHz, DMSO-d6) δ 11.29 (br. s., 1H), 8.52-8.66 (m, 2H), 8.04 (d, J=2.93 Hz, 1H), 7.77-7.90 (m, 2H), 7.71 (s, 1H), 7.44 (dd, J=2.93, 8.97 Hz, 1H), 7.09 (d, J=8.97 Hz, 1H), 3.80 (s, 3H). LCMS (ESI) m/z 285 (MH+)

### (4-Benzofuran-2-yl-thiazol-2-yl)-(5-methoxy-pyridin-2-yl)-amine (IND121)

(5-Methoxy-pyridin-2-yl)-thiourea was reacted with commercially available 1-benzofuran-2-yl-2-bromo-ethanone, according to the general procedure to afford the title compound in 51% yield; mp 185-186° C. 1H NMR (400 MHz, DMSO-d6) δ 11.42 (s, 1H), 8.05 (d, J=2.75 Hz, 1H), 7.67 (d, J=7.32 Hz, 1H), 7.60 (d, J=8.06 Hz, 1H), 7.45 (dd, J=2.93, 8.97 Hz, 1H), 7.39 (s, 1H), 7.21-7.36 (m, 2H), 7.08 (s, 1H), 7.06 (s, 1H), 3.81 (s, 3H). LCMS (ESI) m/z 324 (MH-0

### (4-Biphenyl-4-yl-thiazol-2-yl)-(5-methoxy-pyridin-2-yl)-amine (IND122)

(5-Methoxy-pyridin-2-yl)-thiourea was reacted with commercially available 1-biphenyl-4-yl-2-bromo-ethanone, according to the general procedure to afford the title compound in 68% yield; mp 212-213° C. 1H NMR (400 MHz, DMSO-d6) δ 11.24 (s, 1H), 8.04 (d, J=2.93 Hz, 1H), 7.97-8.02 (m, 2H), 7.69-7.75 (m, 4H), 7.41-7.51 (m, 4H), 7.38 (d, J=7.33 Hz, 1H), 7.08-7.13 (m, 1H), 3.81 (s, 3H). LCMS (ESI) m/z 360 (MH+)

### (4-Benzofuran-2-yl-thiazol-2-yl)-(6-methyl-pyridin-2-yl)-amine (IND22)

(6-Methyl-pyridin-2-yl)-thiourea was reacted with commercially available 1-benzofuran-2-yl-2-bromo-ethanone, according to the general procedure to afford the title compound in 70% yield; mp 199-200° C. 1H NMR (400 MHz, methanol-d6) δ 8.22 (dd, J=7.60, 8.70 Hz, 1H), 7.59-7.71 (m, 2H), 7.52 (dd, J=0.82, 8.15 Hz, 1H), 7.23-7.38 (m, 5H), 2.86 (s, 3H). 13C NMR (100 MHz, DMSO-d6) δ 161.0, 155.8, 154.8, 153.0, 151.5, 140.7, 139.0, 129.2, 125.2, 123.9, 122.0, 115.9, 111.6, 109.2, 108.2, 102.5, 24.0. LCMS (ESI) m/z 308 (MH+)

### (4-Biphenyl-4-yl-thiazol-2-yl)-(6-methyl-pyridin-2-yl)-amine (IND24)

(6-Methyl-pyridin-2-yl)-thiourea was reacted with commercially available 1-biphenyl-4-yl-2-bromo-ethanone, according to the general procedure to afford the title compound in 85% yield; mp 218-222° C. 1H NMR (400 MHz, DMSO-d6) δ 11.35 (s, 1H), 7.96-8.02 (m, 2H), 7.67-7.76 (m, 4H), 7.55-7.63 (m, 1H), 7.42-7.51 (m, 3H), 7.32-7.40 (m, 1H), 6.90 (d, J=8.24 Hz, 1H), 6.79 (d, J=7.14 Hz, 1H), 2.47 (s, 3H). 13C NMR (100 MHz, DMSO-d6) δ 159.7, 155.1, 151.1, 148.1, 139.7, 138.9, 138.1, 134.0, 129.1, 128.9, 128.9, 127.4, 127.0, 126.8, 126.4, 126.1, 114.9, 107.5, 106.2, 23.4. LCMS (ESI) m/z 344 (MH+)

### (4-Methyl-pyridin-2-yl)-[4-(5-pyridin-2-yl-thiophen-2-yl)-thiazol-2-yl]-amine (IND81)

(4-Methyl-pyridin-2-yl)-thiourea was reacted with commercially available 2-bromo-1-(5-pyridin-2-yl-thiophen-2-yl)-ethanone, according to the general procedure to afford the title compound in 66% yield; mp 185-187° C. 1H NMR (400 MHz, DMSO-d6) δ 11.42 (s, 1H), 8.52 (dt, J=0.82, 4.76 Hz, 1H), 8.16 (d, J=5.13 Hz, 1H), 7.90 (d, J=8.06 Hz, 1H), 7.81 (td, J=1.74, 7.74 Hz, 1H), 7.75 (d, J=4.03 Hz, 1H), 7.50 (d, J=3.85 Hz, 1H), 7.35 (s, 1H), 7.25 (ddd, J=0.92, 4.94, 7.33 Hz, 1H), 6.89 (s, 1H), 6.74-6.82 (m, 1H), 2.28 (s, 3H). 13C NMR (100 MHz, DMSO-d6) δ 159.8, 151.9, 151.8, 149.4, 148.6, 146.1, 143.3, 142.8, 140.8, 137.0, 126.1, 124.3, 122.1, 118.5, 117.6, 110.7, 105.6, 20.7. LCMS (ESI) m/z 351 (MH+)

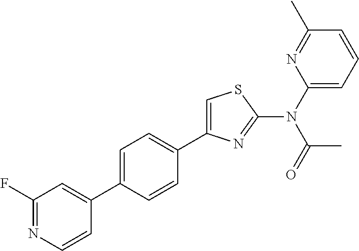 [SMILES: CC(=O)N(c1cccc(C)n1)c1nc(-c2ccc(-c3ccnc(F)c3)cc2)cs1]

N-{4-[4-(2-fluoropyridin-4-yl)phenyl]-1,3-thiazol-2-yl}-N-(6-methylpyridin-2-yl)acetamide N-[4-(4-bromophenyl)-1,3-thiazol-2-yl]-N-(6-methylpyridin-2-ypacetamide (200 mg, 0.515 mmol), (2-fluoropyridin-4-yl)boranediol (105.9 mg, 0.722 mmol), Tris(dibenzylideneacetone)dipalladium(0) (12.4 mg, 0.0135 mmol) and tricyclohexylphosphine (9.2 mg, 0.033 mmol) were dissolved in 1,4 dioxane that had been degassed by bubbling with argon for 2 min. A similarly degassed 1.27 M solution of K₃PO₄ (0.564 ml, 0.716 mmol) was added along with a stirbar. The tube was capped and the contents were further dagassed by 1 min bubbling with argon. The reaction was then subjected to microwave irradiation (150° C. for 0.5 hrs). The reaction residue was partitioned into ethyl acetate, solvent was evaporated and the crude residue was eluted on silica (10 g cartridge) using an automatically generated gradient based of an R_{f} of 0.5 in 1:1 hexane:ethyl acetate. Product, 140 mg (67%, crude yield) was obtained. This could be further purified on silica to give pure product for submission. 1H NMR (400 MHz, CHLOROFORM-d) δ 2.09 (s, 3H) 2.63 (s, 3H) 7.05-7.08 (m, 1H) 7.28-7.36 (m, 3H) 7.51-7.57 (m, 2H) 7.69-7.75 (m, 2H) 7.85 (t, J=7.78 Hz, 1H) 8.20 (d, J=5.13 Hz, 1H); LCMS (ESI) m/z 405 (MH+)

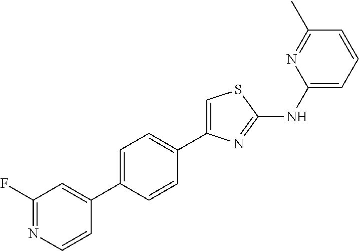 [SMILES: Cc1cccc(Nc2nc(-c3ccc(-c4ccnc(F)c4)cc3)cs2)n1]

4-[4-(2-fluoropyridin-4-yl)phenyl]-N-(6-methylpyridin-2-yl)-1,3-thiazol-2-amine N-{4-[4-(2-fluoropyridin-4-yl)phenyl]-1,3-thiazol-2-yl}-N-(6-methylpyridin-2-yl)acetamide (39.2, 0.092 mmol) was dissolved in 1.00 ml 1,4 dioxane in a microwave vial with a stirbar. Saturated K₂CO₃ (0.3 ml) was added, and the reaction was subjected to microwave irradiation (150° C. for 0.5 hrs). Water (2 ml) and ethyl acetate was added to the reaction mixture, and the product partitioned into the organic layer. The water layer was washed again with ethyl acetate. The combined organics were dried with brine and MgSO₄, filtered and concentrated to afford the product (19 mg, 57%) 1H NMR (400 MHz, DMSO-d6) δ 2.48 (s, 3H) 6.80 (d, J=7.33 Hz, 1H) 6.91 (d, J=8.24 Hz, 1H) 7.57-7.64 (m, 3H) 7.76 (dt, J=5.13, 1.83 Hz, 1H) 7.93-7.98 (m, 2H) 8.04-8.09 (m, 2H) 8.31 (d, J=5.31 Hz, 1H) 11.38 (s, 1H); LCMS (ESI) m/z 363 (MH+)

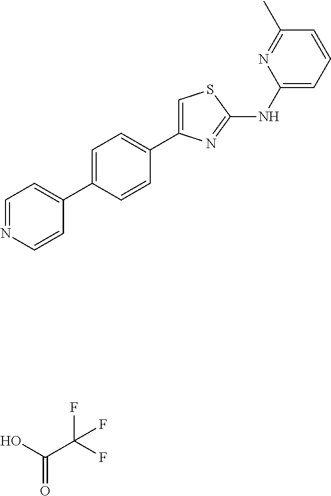 [SMILES: Cc1cccc(Nc2nc(-c3ccc(-c4ccncc4)cc3)cs2)n1 ; O=C(O)C(F)(F)F]

N-(6-methylpyridin-2-yl)-4-[4-(pyridin-4-yl)phenyl]-1,3-thiazol-2-amine trifluoroacetate salt N-[4-(4-bromophenyl)-1,3-thiazol-2-yl]-N-(6-methylpyridin-2-yl)acetamide (162 mg, 0.417 mmol) was reacted with pyridin-4-ylboranediol (76.8 mg, 0.624 mmol) under identical conditions as used for N-{4-[4-(2-fluoropyridin-4-yl)phenyl]-1,3-thiazol-2-yl}-N-(6-methylpyridin-2-yl)acetamide above to afford a precipitate which was filtered and triturated with methanol. This precipitate was dissolved in 3 ml 95% DMSO/water and filtered through a 0.02 micron Anatop filter. The DMSO solution was then eluted on prep HPLC to afford the trifluoroacetate salt (21.2 mg, 11%)

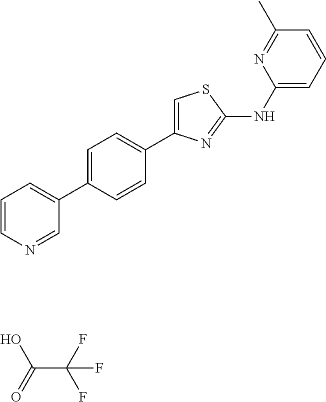 [SMILES: Cc1cccc(Nc2nc(-c3ccc(-c4cccnc4)cc3)cs2)n1 ; O=C(O)C(F)(F)F]

N-(6-methylpyridin-2-yl)-4-[4-(pyridin-3-yl)phenyl]-1,3-thiazol-2-amine, trifluoroacetate salt N-[4-(4-bromophenyl)-1,3-thiazol-2-yl]-N-(6-methylpyridin-2-yl)acetamide (100 mg, 0.258 mmol) was reacted with pyridin-3-ylboranediol (47.5 mg, 0.387 mmol) as above for 6-methyl-N-{4-[4-(pyridin-4-yl)phenyl]-1,3-thiazol-2-yl}pyridin-2-amine. Product was obtained after preparative HPLC as the TFA salt (27 mg, 22%). 1H NMR (400 MHz, DMSO-d6) δ 2.48 (s, 3H) 6.81 (d, J=7.14 Hz, 1H) 6.91 (d, J=8.24 Hz, 1H) 7.52-7.67 (m, 2H) 7.79 (dd, J=8.15, 5.22 Hz, 1H) 7.89 (d, J=8.61 Hz, 2H) 8.08 (d, J=8.42 Hz, 2H) 8.50 (d, J=7.69 Hz, 1H) 8.72 (d, J=4.94 Hz, 1H) 9.12 (br. s., 1H) 1.37 (br. s., 1H); LCMS (ESI) m/z 345 (MH+)

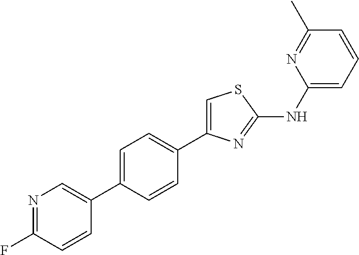 [SMILES: Cc1cccc(Nc2nc(-c3ccc(-c4ccc(F)nc4)cc3)cs2)n1]

### 4-[4-(6-fluoropyridin-3-yl)phenyl]-N-(6-methylpyridin-2-yl)-1,3-thiazol-2-amine

N-[4-(4-bromophenyl)-1,3-thiazol-2-yl]-N-(6-methylpyridin-2-ypacetamide (100 mg, 0.258 mmol) was reacted with (6-fluoropyridin-3-yl)boranediol (54.2 mg, 0.387 mmol) under identical conditions as used for N-{4-[4-(2-fluoropyridin-4-yl)phenyl]-1,3-thiazol-2-yl}-N-(6-methylpyridin-2-yl)acetamide above to afford a precipitate which was filtered and triturated with methanol. This precipitate was dissolved in 3 ml DMSO and filtered through a 0.02 micron Anatop filter. The filtrate was then precipitated from 50 ml ice water to afford product, 21.8 mg (23%). 1H NMR (400 MHz, DMSO-d6) δ 2.48 (s, 3H) 6.80 (d, J=7.51 Hz, 1H) 6.90 (d, J=8.24 Hz, 1H) 7.30 (dd, J=8.42, 2.93 Hz, 1H) 7.53 (s, 1H) 7.57-7.66 (m, 1H) 7.75-7.84 (m, 2H) 7.99-8.08 (m, 2H) 8.34 (td, J=8.24, 2.75 Hz, 1H) 8.61 (d, J=2.56 Hz, 1H) 11.36 (s, 1H); LCMS (ESI) m/z 363 (MH+)

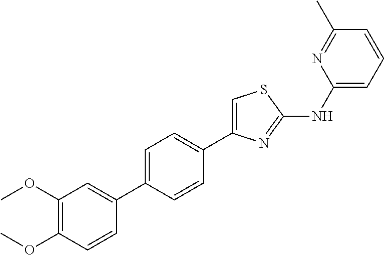 [SMILES: COc1ccc(-c2ccc(-c3csc(Nc4cccc(C)n4)n3)cc2)cc1OC]

### 4-[4-(3,4-dimethoxyphenyl)phenyl]-N-(6-methylpyridin-2-yl)-1,3-thiazol-2-amine

N-[4-(4-bromophenyl)-1,3-thiazol-2-yl]-N-(6-methylpyridin-2-yl)acetamide (100 mg, 0.258 mmol) was reacted with (3,4-dimethoxyphenyl)boranediol (70.3 mg, 0.387 mmol) under identical conditions as used for N-{-4-[4-(2-fluoropyridin-4-yl)phenyl]-1,3-thiazol-2-yl}-N-(6-methylpyridin-2-yl)acetamide above to afford a precipitate and a filtrate, which was concentrated. The precipitate and the concentrated filtrate were found to contain a mixture of both acetylated and unacetylated product. Accordingly, they were hydrolysed by re-suspending in 1,4 dioxane (2 ml), saturated K₂CO₃ (0.5 ml) and subjecting to microwave irradiation (150° C. for 0.5 hrs). After hydrolysis, a precipitate was filtered on paper, washed with water and then diethyl ether. The precipitate was taken up in 3 ml DMSO and filtered through a 0.2 micron Anatop filter then precipitated out from 40 ml ice water. The precipitate was filtered on paper, washed with methanol then diethyl ether to afford final product 28.3 mg (27%). 1H NMR (400 MHz, DMSO-d6) δ 2.48 (s, 3H) 3.80 (s, 3H) 3.86 (s, 3H) 6.79 (d, J=7.33 Hz, 1H) 6.90 (d, J=8.24 Hz, 1H) 7.04 (d, J=8.24 Hz, 1H) 7.21-7.31 (m, 2H) 7.45 (s, 1H) 7.53-7.64 (m, 1H) 7.66-7.77 (m, 2H) 7.89-8.03 (m, 2H) 11.35 (s, 1H); LCMS (ESI) m/z 404 (MH+)

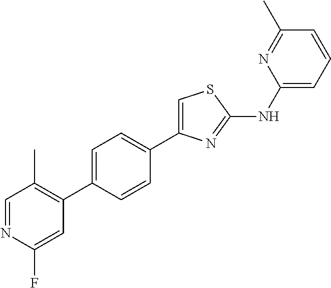 [SMILES: Cc1cccc(Nc2nc(-c3ccc(-c4cc(F)ncc4C)cc3)cs2)n1]

4-[4-(2-fluoro-5-methylpyridin-4-yl)phenyl]-N-(6-methylpyridin-2-yl)-1,3-thiazol-2-amine 4-(4-bromophenyl)-N-(6-methylpyridin-2-yl)-1,3-thiazol-2-amine (50 mg, 0.145 mmol) was reacted with 2-fluoro-5-methyl-4-(4,4,5,5-tetramethyl-1,3,2-dioxaborolan-2-yl)pyridine (51.4 mg, 0.217 mmol) under identical conditions as used for N-{4-[4-(2-fluoropyridin-4-yl)phenyl]-1,3-thiazol-2-yl}-N-(6-methylpyridin-2-yl)acetamide above to afford a precipitate. This was filtered on paper, re-dissolved in 1.4 ml DMSO and gently swirled with 70 mg Quadrapure MPA resin overnight. The resin was then removed by filtration and the DMSO solution was precipitated from ice water to afford product, 3.7 mg (6.7%). 1H NMR (400 MHz, DMSO-d6) δ 2.28 (s, 3H) 2.48 (s, 3H) 6.80 (d, J=7.33 Hz, 1H) 6.91 (d, J=8.24 Hz, 1H) 7.12 (s, 1H) 7.48-7.57 (m, 3H) 7.61 (t, J=7.78 Hz, 1H) 8.04 (d, J=8.06 Hz, 2H) 8.18 (s, 1H) 11.36 (s, 1H); LCMS (ESI) m/z 377 (MH+)

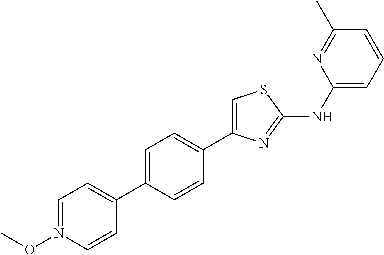

4-[4-(6-methoxypyridin-3-yl)phenyl]-N-(6-methylpyridin-2-yl)-1,3-thiazol-2-amine 4-(4-bromophenyl)-N-(6-methylpyridin-2-yl)-1,3-thiazol-2-amine (50 mg, 0.145 mmol) was reacted with (6-methoxypyridin-3-yl)boranediol (33.2 mg, 0.217 mmol) exactly as above for N-{4-[4-(2-fluoro-5-methylpyridin-4-yl)phenyl]-1,3-thiazol-2-yl}-6-methylpyridin-2-amine to afford product, 15.8 mg (29%). 1H NMR (400 MHz, DMSO-d6) δ 11.35 (s, 1H), 8.48-8.60 (m, 1H), 8.07 (ddd, J=0.82, 2.61, 8.65 Hz, 1H), 7.96-8.02 (m, J=7.87 Hz, 2H), 7.70-7.76 (m, J=8.06 Hz, 2H), 7.57-7.63 (m, 1H), 7.48 (s, 1H), 6.87-6.95 (m, 2H), 6.79 (dd, J=0.55, 7.33 Hz, 1H), 3.91 (s, 3H), 2.48 (s, 3H); LCMS (ESI) m/z 375 (MH+)

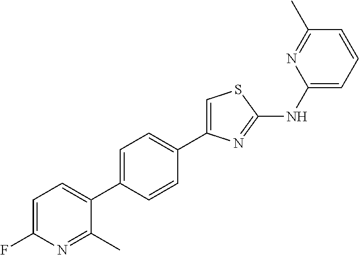 [SMILES: Cc1cccc(Nc2nc(-c3ccc(-c4ccc(F)nc4C)cc3)cs2)n1]

4-[4-(6-fluoro-2-methylpyridin-3-yl)phenyl]-N-(6-methylpyridin-2-yl)-1,3-thiazol-2-amine 4-(4-bromophenyl)-N-(6-methylpyridin-2-yl)-1,3-thiazol-2-amine (50 mg, 0.145 mmol) was reacted with (6-fluoro-2-methylpyridin-3-yl)boranediol (33.6 mg, 0.217 mmol) as above for N-{4-[4-(2-fluoro-5-methylpyridin-4-yl)phenyl]-1,3-thiazol-2-yl}-6-methylpyridin-2-amine to afford a crude product precipitate which was filtered on paper. This was eluted on silica (12 g cartridge) using an automatically generated gradient based of an R_{f} of 0.2 in 85:15 hexane:ethyl acetate, to afford product, 26.2 mg (48%). 1H NMR (400 MHz, CHLOROFORM-d) δ 10.08 (br. s., 1H), 7.97 (d, J=8.06 Hz, 2H), 7.61 (t, J=8.06 Hz, 1H), 7.19-7.45 (m, 4H), 7.03-7.19 (m, 1H), 6.81 (dd, J=3.30, 8.24 Hz, 1H), 6.68 (d, J=7.33 Hz, 1H), 6.33 (d, J=8.06 Hz, 1H), 2.55 (s, 3H), 2.43 (s, 3H); LCMS (ESI) m/z 377 (MH+)

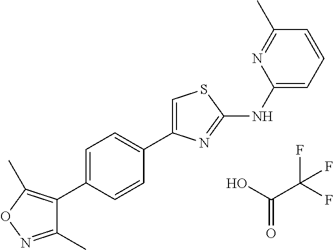 [SMILES: Cc1cccc(Nc2nc(-c3ccc(-c4c(C)noc4C)cc3)cs2)n1 ; O=C(O)C(F)(F)F]

4-[4-(3,5-dimethyl-1,2-oxazol-4-yl)phenyl]-N-(6-methylpyridin-2-yl)-1,3-thiazol-2-amine trifluoroacetate salt 4-(4-bromophenyl)-N-(6-methylpyridin-2-yl)-1,3-thiazol-2-amine (50 mg, 0.145 mmol) was reacted with (3,5-dimethyl-1,2-oxazol-4-yl)boranediol (29.7 mg, 0.211 mmol) as above for N-{4-[4-(2-fluoro-5-methylpyridin-4-yl)phenyl]-1,3-thiazol-2-yl}-6-methylpyridin-2-amine to crude product which was partitioned between ethyl acetate and water. The organic layer was dried and concentrated. Crude product was eluted on silica (12 g cartridge) using an automatically generated gradient based of an R_{f} of 0.2 in 4:1 hexane:ethyl acetate, to afford product, 48.4 mg. This was further purified by preparative HPLC to afford the TFA salt 43.1 mg (62%). 1H NMR (400 MHz, CHLOROFORM-d) δ 7.84-7.90 (m, 2H), 7.70 (t, J=7.87 Hz, 1H), 7.38-7.43 (m, 2H), 7.31 (d, J=7.69 Hz, 1H), 6.96 (t, J=3.66 Hz, 2H), 2.62 (s, 3H), 2.46 (s, 3H), 2.32 (s, 3H); LCMS (ESI) m/z 363 (MH+)

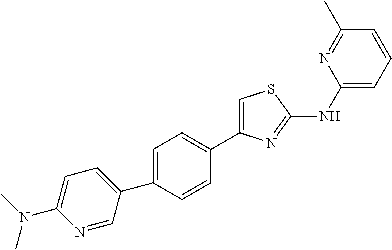 [SMILES: Cc1cccc(Nc2nc(-c3ccc(-c4ccc(N(C)C)nc4)cc3)cs2)n1]

4-{-4-[6-(dimethylamino)pyridin-3-yl]phenyl}-N-(6-methylpyridin-2-yl)-1,3-thiazol-2-amine amine 4-(4-bromophenyl)-N-(6-methylpyridin-2-yl)-1,3-thiazol-2-amine (50 mg, 0.145 mmol) was reacted with [6-(dimethylamino)pyridin-3-yl]boranediol (36.0 mg, 0.217 mmol) under identical conditions as used for 530501 above to afford a precipitate. This was filtered on paper, re-dissolved in 1.8 ml DMSO and gently swirled with 70 mg Quadrapure MPA resin overnight. A precipitate formed with the resin, this was clarified by brief heating (heat gun). The resin was then removed by filtration and the DMSO solution was precipitated from ice water to afford product, 17.5 mg (33%). 1H NMR (400 MHz, DMSO-d6) δ 11.33 (s, 1H), 8.49 (d, J=2.56 Hz, 1H), 7.92-7.97 (m, 2H), 7.88 (dd, J=2.66, 8.88 Hz, 1H), 7.64-7.69 (m, J=8.42 Hz, 2H), 7.57-7.62 (m, 1H), 7.42 (s, 1H), 6.90 (d, J=8.24 Hz, 1H), 6.79 (d, J=7.33 Hz, 1H), 6.73 (d, J=8.97 Hz, 1H), 3.07 (s, 6H), 2.48 (s, 3H); LCMS (ESI) m/z 388 (MH+)

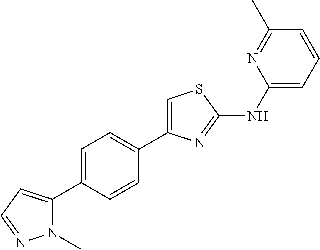 [SMILES: Cc1cccc(Nc2nc(-c3ccc(-c4ccnn4C)cc3)cs2)n1]

4-[4-(1-methyl-1H-pyrazol-5-yl)phenyl]-N-(6-methylpyridin-2-yl)-1,3-thiazol-2-amine 4-(4-bromophenyl)-N-(6-methylpyridin-2-yl)-1,3-thiazol-2-amine (50 mg, 0.145 mmol) was reacted with (1-methyl-1H-pyrazol-5-yl)boranediol (36.5 mg 0.290 mmol) under conditions as used for N-{-4-[4-(2-fluoropyridin-4-yl)phenyl]-1,3-thiazol-2-yl}-N-(6-methylpyridin-2-yl)acetamide except using increased catalyst: tricyclohexyl phosphine (6.0 mg, 0.21 mmol) and tris(dibenzylideneacetone)dipalladium(0) (8.0 mg, 0.087 mmol) and microwave irradiation (150° C. for 1.0 hr). A crude product was partitioned between ethyl acetate and water. The organic layer was dried and concentrated. This was eluted on silica (12 g cartridge) using an automatically generated gradient based of an R_{f} of 0.5 in 1:1 hexane:ethyl acetate, to afford product (31.5 mg, 62%). 1H NMR (400 MHz, CHLOROFORM-d) δ 9.79 (s, 1H), 7.96 (d, J=8.24 Hz, 2H), 7.51 (d, J=1.83 Hz, 1H), 7.42 (d, J=8.42 Hz, 2H), 7.30 (t, J=7.78 Hz, 1H), 7.10 (s, 1H), 6.68 (d, J=7.33 Hz, 1H), 6.35 (d, J=8.24 Hz, 1H), 6.31 (d, J=1.83 Hz, 1H), 3.87 (s, 3H), 2.54 (s, 3H); LCMS (ESI) m/z 348 (MH+)

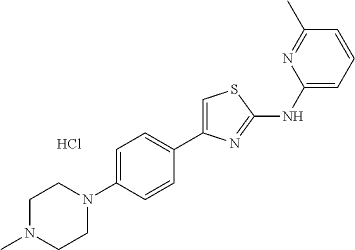 [SMILES: Cc1cccc(Nc2nc(-c3ccc(N4CCN(C)CC4)cc3)cs2)n1 ; Cl]

4-[4-(4-methylpiperazin-1-yl)phenyl]-N-(6-methylpyridin-2-yl)-1,3-thiazol-2-amine hydrochloride 4-(4-bromophenyl)-N-(6-methylpyridin-2-yl)-1,3-thiazol-2-amine (500 mg, 1.45 mmol), N-methyl piperazine (311 mg, 0.345 ml, 3.11 mmol), sodium tert-butoxide (350 mg, 3.60 mmol), Tris(dibenzylideneacetone)dipalladium(0) (63 mg, 0.068 mmol) and (−)BINAP (63 mg, 0.1 mmol) were dissolved in dry dimethylformamide that had been degassed by bubbling with argon for 2 min. The tube was capped and the contents were further degassed by 1 min bubbling with argon. The reaction was then subjected to microwave irradiation (140° C. for 1.0 hr). The reaction residue was diluted with ethyl acetate and shaken with 50% saturated sodium bicarbonate (2×). The combined organic layers were dried with bring and MgSO₄ was evaporated and the crude residue was eluted on silica (40 g cartridge) using an automatically generated gradient based of an R_{f} of 0.15 in 9:1 methylene chloride:methanol. Product was taken up in 8 ml 1:1 dry methanol: dichloromethane, and 1M HCl in ether was added until a precipitate formed. This was filtered on paper to afford the product as the HCl salt (34 mg, 5.4%). 1H NMR (400 MHz, DEUTERIUM OXIDE) δ 8.01 (t, J=8.15 Hz, 1H), 7.33 (d, J=8.24 Hz, 2H), 7.04-7.13 (m, 2H), 6.90 (d, J=8.79 Hz, 1H), 6.82 (d, J=8.42 Hz, 2H), 3.80 (d, J=13.37 Hz, 2H), 3.65 (d, J=12.27 Hz, 2H), 3.18-3.29 (m, 2H), 3.05 (m, 2H), 2.98 (s, 3H), 2.46 (s, 3H); LCMS (ESI) m/z 366 (MH+)

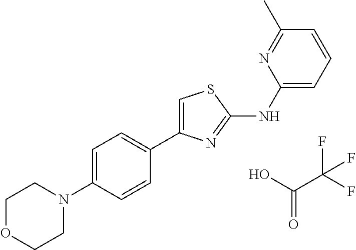 [SMILES: Cc1cccc(Nc2nc(-c3ccc(N4CCOCC4)cc3)cs2)n1 ; O=C(O)C(F)(F)F]

N-(6-methylpyridin-2-yl)-4-[4-(morpholin-4-yl)phenyl]-1,3-thiazol-2-amine 4-(4-bromophenyl)-N-(6-methylpyridin-2-yl)-1,3-thiazol-2-amine (200 mg, 0.580 mmol) and morpholine (126 mg, 0.126 ml, 1.45 mmol), were reacted as above for 6-methyl-N-{4-[4-(4-methylpiperazin-1-yl)phenyl]-1,3-thiazol-2-yl}pyridin-2-amine. The crude reaction residue was eluted on silica (25 g cartridge) using an automatically generated gradient based of an R_{f} of 0.4 in 1:1 hexane:ethyl acetate. The material was further purified by preparative HPLC to afford the trifluoroacetate salt (5.8 mg, 2.1%). 1H NMR (400 MHz, CHLOROFORM-d) δ 7.68-7.72 (m, 2H), 7.63-7.68 (m, 1H), 7.20 (br. s., 1H), 6.96-7.02 (m, 2H), 6.93 (d, J=7.51 Hz, 1H), 6.70 (s, 1H), 3.85-3.91 (m, 4H), 3.22-3.29 (m, 4H), 2.60 (s, 3H); LCMS (ESI) m/z 353 (MH+)

### [4-(4-Bromo-phenyl)-thiazol-2-yl]-(6-methyl-pyridin-2-yl)-amine

An ethanolic solution of thiourea prepared as described (AGG, J.M. Chem. 2011) (1 mmol) and commercially available 2-bromo-1-(4-bromo-phenyl)-ethanone (1 mmol, 1.1 equiv.) was stirred at room temperature overnight. Once the reaction was done (by LC/MS) the reaction mixture was poured into water-ice (20 mL) and stirred for another 30 min. A saturated solution of Na₂CO₃ was added to produce a solution of pH ˜8. The aminothiazole product precipitated from this solution and it was collected by filtration and washed with water. The crude product was purified by column chromatography on silica gel (40 to 100% ethyl acetate-hexane). Relevant fractions were collected and concentrated to afford the desired product in 80% yield, with purity of >95% as determined by 1H-NMR. 1H NMR (400 MHz, DMSO-d6) δ 11.35 (s, 1H), 7.82-7.89 (m, 2H), 7.56-7.64 (m, 3H), 7.49 (s, 1H), 6.88 (d, J=8.24 Hz, 1H), 6.79 (d, J=7.33 Hz, 1H), 2.47 (s, 3H); LCMS (ESI) m/z 347 (MH+)

### N-[4-(4-Bromo-phenyl)-thiazol-2-yl]-N-(6-methyl-pyridin-2-yl)-acetamide

Acetic anhydride (5 mL) was added to [4-(4-Bromo-phenyl)-thiazol-2-yl]-(6-methyl-pyridin-2-yl)-amine (0.8 gr, 2.3 mmol). The reaction was heated to 100° C. for 4 hours and then it was let to cool down to room temperature. Water was added and the product was extracted in EtOAc. The solvent was evaporated and the crude product was purified by column chromatography on silica gel (30 to 60% ethyl acetate-hexane). Relevant fractions were collected and concentrated to afford the desired product in 81% yield. 1H NMR (400 MHz, DMSO-d6) δ 8.01 (t, J=7.78 Hz, 1H), 7.81 (s, 1H), 7.45-7.59 (m, 6H), 2.53 (s, 3H), 1.98 (s, 3H)

### [4-(4-Bromo-phenyl)-thiazol-2-yl]-(6-methyl-pyridin-2-yl)-amine

An ethanolic solution of thiourea prepared as described (AGG, J. M. Chem. 2011) (1 mmol) and commercially available 2-bromo-1-(4-bromo-phenyl)-ethanone (1 mmol, 1.1 equiv.) was stirred at room temperature overnight. Once the reaction was done (by LC/MS) the reaction mixture was poured into water-ice (20 mL) and stirred for another 30 min. A saturated solution of Na₂CO₃ was added to produce a solution of pH 8. The aminothiazole product precipitated from this solution and it was collected by filtration and washed with water. The crude product was purified by column chromatography on silica gel (˜40 to 100% ethyl acetate-hexane). Relevant fractions were collected and concentrated to afford the desired product in 80% yield, with purity of >95% as determined by 1H-NMR. 1H NMR (400 MHz, DMSO-d6) δ 11.35 (s, 1H), 7.82-7.89 (m, 2H), 7.56-7.64 (m, 3H), 7.49 (s, 1H), 6.88 (d, J=8.24 Hz, 1H), 6.79 (d, J=7.33 Hz, 1H), 2.47 (s, 3H); LCMS (ESI) m/z 347 (MH+)

Representative of “Standard Coupling”

N,N-dimethyl-5-{4-{2-[(6-methylpyridin-2-yl)amino]-1,3-thiazol-4-yl}phenyl)pyridin-2-amine: A solution of 6-methyl-N-{4-[4-(tetramethyl-1,3,2-dioxaborolan-2-yl)phenyl]-1,3-thiazol-2-yl}pyridin-2-amine (300 mg, 0.76 mmol), 2-dimethylamino-5-bromopyridine (199 mg, 0.99 mmol), Pd(dppf)₂Cl₂ (62 mg, 0.08 mmol) and K₂CO3 (316 mg, 2.29 mmol) in dioxane (25 ml) and water (5 ml) was heated to 80° C. under N₂ overnight. The mixture was evaporated to remove solvents and the residue was treated with water and DCM, dried with Na₂SO₄, evaporated and purified by HPLC prep to give N,N-dimethyl-5-(4-{2-[(6-methylpyridin-2-yl)amino]-1,3-thiazol-4-yl}phenyl)pyridin-2-amine.

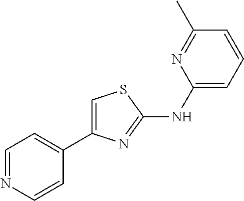 [SMILES: Cc1cccc(Nc2nc(-c3ccncc3)cs2)n1]

### 6-methyl-N-[4-(pyridin-4-yl)-1,3-thiazol-2-yl]pyridin-2-amine

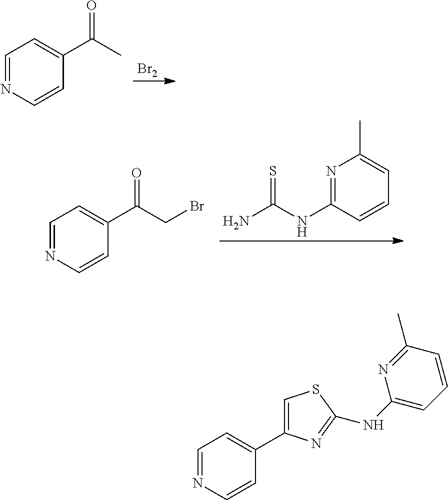

To a solution of starting ketone (1.0 g, 8.3 mmol) and HBr/AcOH (33%, 10 ml) was added Br₂ (1.3 g, 8.3 mmol) dropwise at RT. Then the mixture was stirred at RT overnight. Diluted with Et2O and filtered to give alpha-bromoketone as HBr salt (2.2 g, 95%). A mixture of bromoketone (2.2 g, 7.8 mmol) and thiourea (1.3 g, 7.8 mmol) in ACN (30 mL) was stirred at reflux for 1 h, then concentrated and purified with prep. HPLC to give 6-methyl-N-[4-(pyridin-4-yl)-1,3-thiazol-2-yl]pyridin-2-amine as a yellow solid (76 mg, 4%). ¹H NMR (400 MHz, DMSO-d₆): δ 2.51 (3H, s), 6.81-6.92 (2H, m), 7.61-7.65 (1H, m), 7.81-7.86 (3H, m), 8.61-8.62 (2H, m), 11.47 (1H, s); LCMS (mobile phase: 10%-95% Acetonitrile-Water-0.02% NH4Ac) purity is 97.6%, Rt=3.506 min; MS Calcd.:268; MS Found: 269 (M⁺+1).

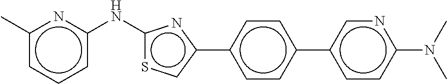

### N,N-dimethyl-5-(4-{2-[(6-methylpyridin-2-yl)amino]-1,3-thiazol-4-yl}phenyl)pyridin-2-amine

To sealed tube was added 5-bromo-2-fluoropyridine (825 mg, 5.1 mmol), dimethylamine/water (7.0 mL, 33%) and THF (3.0 mL), then sealed and heated to 110° C. overnight. The reaction mixture was cooled to RT and extracted with EA, the organic layers were combined and washed with brine, then dried over Na₂SO₄ and concentrated to give compound 2-dimethylamino-5-bromopyridine as yellow oil (880 mg, yield 86%). Coupling under standard conditions gave 127 mg from 300 mg of 6-methyl-N-{4-[4-(tetramethyl-1,3,2-dioxaborolan-2-yl)phenyl]-1,3-thiazol-2-yl}pyridin-2-amine, yellow solid, 43% yield. ¹H NMR (400 MHz, DMSO-d₆): δ 2.47 (3H, s), 3.08 (6H, s), 6.73-6.75 (2H, m), 6.85-6.87 (1H, m), 7.39 (1H, s), 7.55-7.59 (1H, m), 7.65-7.68 (2H, m), 7.88-7.90 (1H, m), 7.92-7.96 (2H, m), 8.49 (1H, s); LCMS (mobile phase: 5%-95% Acetonitrile-Water-0.02% NH4Ac) purity is 96.23%, Rt=2.976 min; MS Calcd.:387; MS Found: 388.1 (M⁺+1).

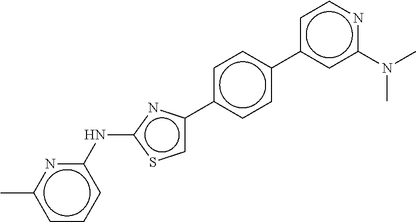

### N,N-dimethyl-4-(4-{2-[(6-methylpyridin-2-yl)amino]-1,3-thiazol-4-yl}phenyl)pyridin-2-amine

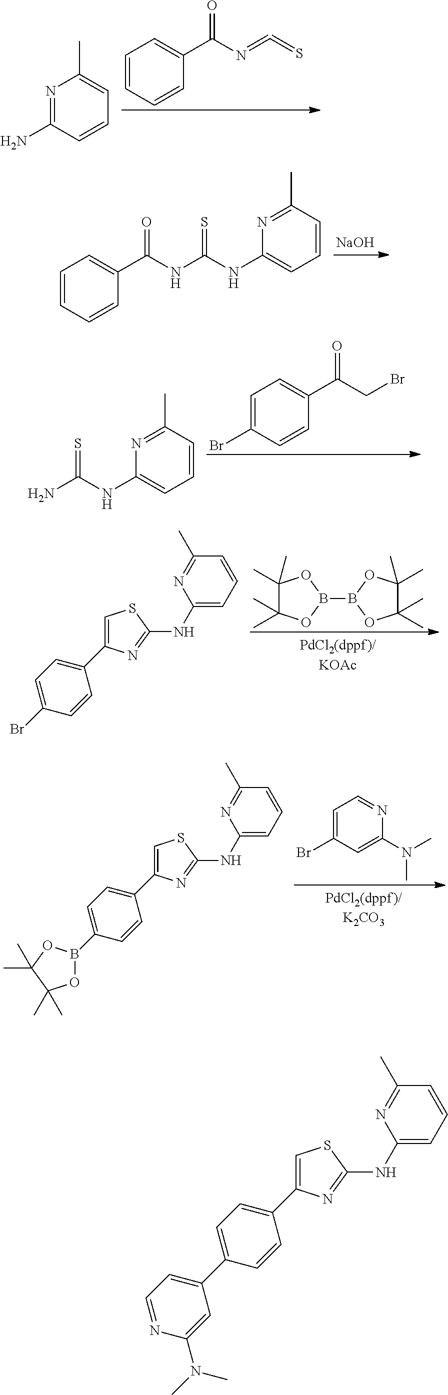

To the solution of benzylisothiocyanate (83.0 g, 509.3 mmol) in acetone (700 mL) was added compound 6-methylpyridin-2-amine (50 g, 463.0 mmol) in acetone (600 ml) dropwise, then the reaction mixture was stirred at reflux for 3 h. The reaction mixture was poured on to crushed ice, then filtered and washed with water, water/MeOH (1:1) and MeOH to give 1-benzoyl-3-(6-methylpyridin-2-yl)thiourea as a yellow solid (100.1 g, yield 80%). To a solution of 1-benzoyl-3-(6-methylpyridin-2-yl)thiourea (60 g, 221.4 mmol) in THF (1000 ml) was added 2N NaOH (243.5 ml), then heated at reflux for 3 h. Cooled to RT and filtered to give (6-methylpyridin-2-yl)thiourea as a white solid (34.1 g, yield 92%). A mixture of (6-methylpyridin-2-yl)thiourea (13.2 g, 79.16 mmol) and 2-bromo-1-(4-bromophenyl)ethan-1-one (22 g, 79.16 mmol) in ethanol (300 mL) was stirred at reflux for 3 h, then concentrated and purified with silica gel column to give N-[4-(4-bromophenyl)-1,3-thiazol-2-yl]-6-methylpyridin-2-amine as a yellow solid (14.3 g, 53%). A solution of N-[4-(4-bromophenyl)-1,3-thiazol-2-yl]-6-methylpyridin-2-amine (5 g. 14.5 mmol), Bis(pinacolato)diboron (4.8 g, 18.8 mmol), Pd(dppf)₂Cl₂ (1.2 mg, 1.5 mmol) and AcOK (4.3 g, 43.3 mmol) in dioxane (100 ml) was heated to 80 under N₂ overnight. The mixture was evaporated to give (6-methyl-N-{4-[4-(tetramethyl-1,3,2-dioxaborolan-2-yl)phenyl]-1,3-thiazol-2-yl}pyridin-2-amine). Coupling (200 mg) with 2-dimethylamino-4-bromopyridine under standard conditions gave 33 mg from 200 mg of, yellow solid, 17% yield.

¹H NMR (400 MHz, DMSO-d₆): δ2.57 (3H, s), 3.16 (6H, s), 6.50-6.52 (1H, m), 6.72-6.74 (2H, m), 6.80 (1H, m), 7.40-7.42 (1H, m), 7.42-7.44 (2H, m), 7.96-7.98 (2H, m), 8.22-8.24 (1H, m), 9.01 (1H, s); LCMS (mobile phase: 5%-95% Acetonitrile-Water-0.02% NH4Ac) purity is 99.45%, Rt=3.101 min; MS Calcd.:387; MS Found: 388.1 (M⁺+1).

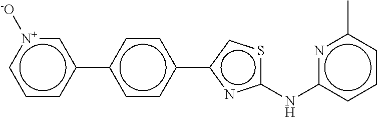

3-(4-{2-[(6-methylpyridin-2-yl)amino]-1,3-thiazol-4-yl}phenyl)pyridin-1-ium-1-olate: Gave 42 mg from 200 mg of (6-methyl-N-{4-[4-(tetramethyl-1,3,2-dioxaborolan-2-yl)phenyl]-1,3-thiazol-2-yl}pyridin-2-amine) coupled in the standard fashion to m-bromopyridine-N-oxide, yellow solid, 23% yield.

¹H NMR (400 MHz, DMSO-d₆): δ2.50 (3H, s), 6.80-6.81 (1H, m), 6.89-6.91 (1H, m), 7.49-7.61 (3H, m), 7.63 (1H, s), 7.71-7.73 (2H, m), 8.03-8.05 (2H, m), 8.21-8.23 (1H, m), 8.64 (1H, s), 11.38 (1H, s)

LCMS (mobile phase:30%-95% Acetonitrile-Water-0.02% NH4Ac) purity is 93.82%, Rt=2.289 min; MS Calcd.:360; MS Found: 361.1 (M⁺+1).

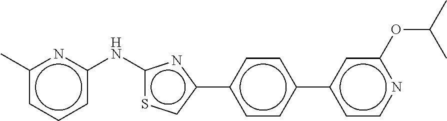

### 6-methyl-N-(4-{4-[2-(propan-2-yloxy)pyridin-4-yl]phenyl}-1,3-thiazol-2-yl)pyridin-2-amine

Coupling of 200 mg of (6-methyl-N-{4-[4-(tetramethyl-1,3,2-dioxaborolan-2-yl)phenyl]-1,3-thiazol-2-yl}pyridin-2-amine) with 4-bromo-2-isopropoxypyridine under standard conditions gave 57 mg yellow solid, 28% yield.

¹H NMR (400 MHz, DMSO-d₆): δ1.35-1.39 (6H, m), 2.55 (3H, s), 5.32-5.39 (1H, m), 6.37-6.39 (1H, m), 6.68-6.70 (1H, m), 6.93 (1H, s), 7.07-7.12 (2H, m), 7.30-7.34 (1H, m), 7.63-7.65 (2H, m), 7.97-7.99 (2H, m), 8.18-8.19 (1H, m), 9.72 (1H, s)

LCMS (mobile phase:60%-95% Acetonitrile-Water-0.02% NH4Ac) purity is 98.98%, Rt=3.738 min; MS Calcd.:402; MS Found: 403.1 (M⁺+1).

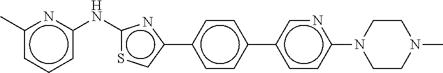

### 6-methyl-N-(4-{4-[6-(4-methylpiperazin-1-yl)pyridin-3-yl]phenyl}-1,3-thiazol-2-yl)pyridin-2-amine

To a solution of 5-bromo-2-fluoropyridine (2.0 g, 11.4 mmol) in acetonitrile (20 mL) was added methylpiperazine (11.4 g, 114 mmol), the resulting mixture was refluxed for 18 h. After concentration, the residue was dissolved in EA (50 mL), washed with water and brine, dried over Na₂SO₄ and concentrated to give 5-Bromo-2-N-Me-piperizinylpyridine product as white solid (2.35 g, yield 81%). Standard coupling gave 128 mg product from 200 mg of (6-methyl-N-{4-[4-(tetramethyl-1,3,2-dioxaborolan-2-yl)phenyl]-1,3-thiazol-2-yl}pyridin-2-amine), yellow solid, 57% yield. 1H NMR (400 MHz, DMSO-d6): δ 2.23 (3H, s), 2.31-2.33 (4H, m), 2.40 (3H,$), 3.52-3.57 (4H, m), 6.76-6.78 (1H, m), 6.86-6.94 (2H, m), 7.42 (1H, s), 7.56-7.59 (1H, m), 7.67-7.69 (2H, m), 7.89-7.97 (3H, m), 8.50-8.52 (1H, m), 11.37 (1H, s).

LCMS (mobile phase: 40%-95% Acetonitrile-Water-0.02% NH4Ac) purity is 98.79%, Rt=3.518 min; MS Calcd.:442; MS Found: 443.2 (M++1)

### 2-N,2-N-dimethyl-6-N-[4-(4-phenylphenyl)-1,3-thiazol-2-yl]pyridine-2,6-diamine

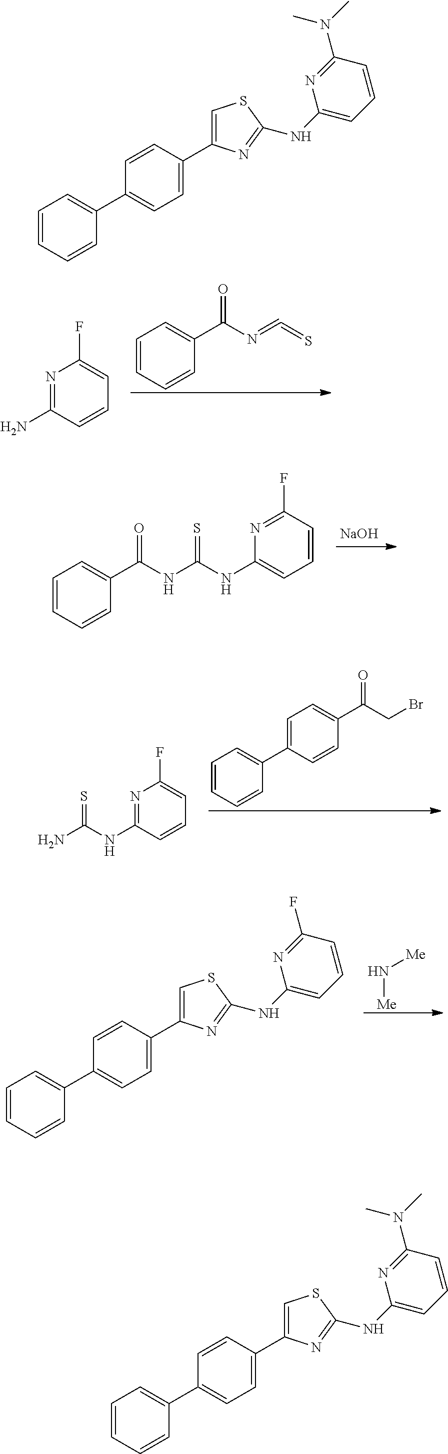

To the solution of acylthiocyanate (3.2 g, 19.6 mmol) in acetone (30 mL) was added 2-fluoro-6-aminopyridine (2.0 g, 17.9 mmol) in acetone (15 ml) dropwise, then the reaction mixture was stirred at reflux for 3 h. The reaction mixture was poured on to crushed ice, then filtered and washed with water to give thiourea as a yellow solid (4.9 g, crude yield 91%). To a solution of thiourea (4.9 g, 17.9 mmol) in THF (72 ml) was added 2N NaOH (18 mL), then heated at reflux for 3 h. Concentrated and dissolved with water, then extracted with DCM, combined and washed with water and brine, then dried over Na2SO4 and concentrated to give the free thiourea as a yellow solid (1.7 g, yield 56%). A mixture of this free thiourea (1.7 g, 10.0 mmol), alphabromoaryl ketone (2.78 g, 10.0 mmol) and NaHCO3 (1.0 g, 12 mmol) in ACN (25 mL) was stirred at reflux for 6 h. Concentrated and dissolved with DCM, filtered and the filtrate was concentrated to give 6-fluoro-N-[4-(4-phenylphenyl)-1,3-thiazol-2-yl]pyridin-2-amine as a yellow solid (1.4 g, 40%). To sealed tube was added 6-fluoro-N-[4-(4-phenylphenyl)-1,3-thiazol-2-yl]pyridin-2-amine (450 mg, 1.3 mmol) and dimethylamine/water (100 mL, 33%), then sealed and heated to 130° C. overnight. The reaction mixture was cooled to RT and extracted with EA, the organic layers were combined and washed with brine, then dried over Na2SO4 and concentrated to give a gray solid, further purified with prep.HPLC to give 2-N,2-N-dimethyl-6-N-[4-(4-phenylphenyl)-1,3-thiazol-2-yl]pyridine-2,6-diamine as a gray solid (54 mg, yield 11%). ¹H NMR (400 MHz, CDC3): δ 3.20 (6H, s), 5.93 (1H, d, J=8.0 Hz), 6.02 (1H, d, J=8.0 Hz), 7.02 (1H, s), 7.30-7.36 (2H, m), 7.43-7.47 (2H, m), 7.63-7.65 (4H, m), 7.92-7.95 (2H, m), 8.52 (1H, s);

LCMS (mobile phase: 70%-95% Acetonitrile-Water-0.02% NH4Ac) purity is 96.0%, Rt=2.713 min; MS Calcd.:372; MS Found: 373 (M⁺+1).

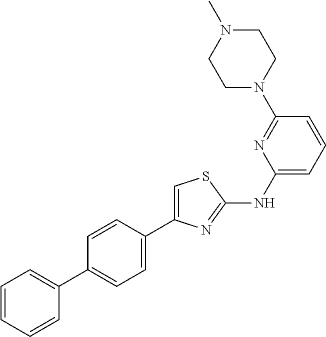 [SMILES: CN1CCN(c2cccc(Nc3nc(-c4ccc(-c5ccccc5)cc4)cs3)n2)CC1]

### 6-(4-methylpiperazin-1-yl)-N-[4-(4-phenylphenyl)-1,3-thiazol-2-yl]pyridin-2-amine

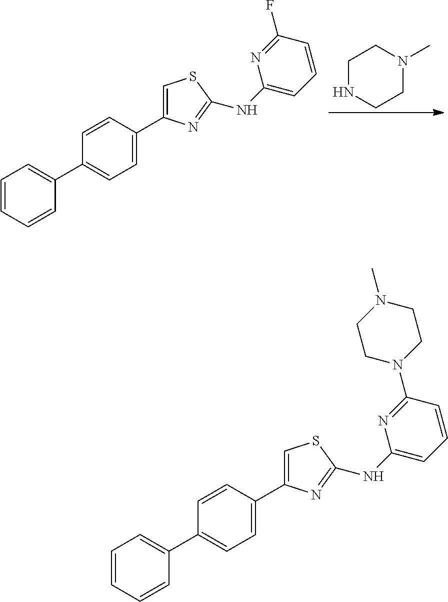

A solution of the previously described 6-fluoro-N-[4-(4-phenylphenyl)-1,3-thiazol-2-yl]pyridin-2-amine (130 mg, 0.4 mmol) in N-methylpiperazine (5 mL) was heated to 120° C. for 4 h. The reaction mixture was added dropwise to water, filtrated to collect the solid. The solid was further purified by prep. HPLC to give 6-(4-methylpiperazin-1-yl)-N-[4-(4-phenylphenyl)-1,3-thiazol-2-yl]pyridin-2-amine as a white solid (69 mg, yield 37%).

¹H NMR (400 MHz, CDCl₃): δ 2.37 (3H, s), 2.56-2.58 (4H, m), 3.69-3.72 (4H, m), 6.07-6.09 (1H, m), 6.19-6.21 (1H, m), 7.02 (1H, s), 7.33-7.46 (4H, m), 7.63-7.65 (4H, m), 7.91-7.93 (2H, m), 8.26-8.32 (1H, s);

LCMS (mobile phase: 20%-95% Acetonitrile-Water-0.02% NH4Ac) purity is 95.6%, Rt=3.293 min; MS Calcd.:427 MS Found: 428.1 (M⁺+1).

### P. Example 15

### Ntiprion Potency and Calculated Physicochemical Parameters

In Vitro ADME Studies.

Permeability (MDR¹-MDCK) and microsome stability data was generated at ADMETRx, Inc. (Kalamazoo, Mich.) as described below.

In Vivo Studies.

Aminothiazole analog (e.g., 4.5 g of 27) was added to 15 mL of pure PEG400, vortexed and sonicated to ensure dissolution, then stored at −4° C. until needed. This highly concentrated PEG400 solution was subsequently diluted to final dosing concentrations, where the volume added was composed of homogenized solid rodent feed, cocoa (taste masking agent), and water. Wild-type FVB mice weigh ˜25 gm and typically drink 20 mL of liquid diet per day, allowing an estimate of daily drug consumption. A single dosing cohort consisted of three mice in a shared cage and liquid diet was provided at the start of the study in sufficient volume to last for the entire three-day trial (˜200 mL). At the end of the three-day dosing period, animals were euthanized by CO₂, followed by collection of plasma (cardiac puncture) and removal of whole brain. Heparinized blood was centrifuged to separate plasma and both plasma and brain samples were stored at −80° C. prior to analysis. Plasma samples were prepared for analysis by precipitating proteins and reconstituting the remaining fraction with HPLC mobile phase. Brain samples were typically prepared by four-fold dilution with water after weighing, then homogenized using bead-beater or Polytron™ resulting in a highly concentrated solution that was further diluted with mobile phase as appropriate in preparation for bioanalytical analysis using LC-MS. Analysis was by LC-MS (Shimadzu dual-HPLC pumps, C18 analytical column, with detection using an Applied Biosystems API-4000 triple quadrupole mass spectrometer). Specific LC-MS methods were developed for each compound analyzed and the stability of the compounds in brain and plasma were demonstrated for the time period of sample handling, workup, and LC-MS analysis.

The antiprion potency (as EC₅₀ values) of AMT compounds was determined as described above. Calculated physicochemical parameters, including tPSA (A), xlogP, and number of hydrogen bond donors and acceptors were determined using SARvision

From the EC₅₀ values and physicochemical parameters for the 34 2-AMT compounds (Tables 1 and 2), we selected 10 analogs for further characterization in vitro. Calculated parameters included total polar surface area (tPSA), xlogP, and number of hydrogen bond donors and acceptors. The calculated values, such as xlogP and PSA, were generally within the range of acceptable values for CNS drugs.

### Q. Example 16

### Solubility

Fasted-State Simulated Intestinal Fluid Solubility of AMTs.

Intestinal solubility was estimated by fasted-state simulated intestinal-fluid solubility (FaSSIF). Aliquots of the 10 mM DMSO stocks were transferred to pH 2 (HCl) buffer, pH 7.4 (phosphate) buffer with and without 0.05% polysorbate 80 (PS80) or FaSSIF to give a target concentration of 250 μM in solute and 2.5% DMSO. After equilibration at room temperature overnight, the solutions were filtered and solute concentration determined by fast gradient HPLC with UV/VIS/MS detection with reference to 1, 5, 10, 50, 100 and 250 μM analytical standards. These analytical standards were prepared from a 500-μM intermediate stock solution made by diluting 10 μl of the 10-mM sample stocks into 190 μA of 50:50 (v/v) ACN/water. Aliquots of 0.4, 2, 4, 20, 40, and 100 μA were transferred in duplicate into a 96-well plate and the volumes were made up to 200 μA per well with ACN/water. The plate was then heat-sealed with a foil sheet. Prior to sample filtration, filters were primed with 600 μA of sample to resolve potential adsorption problems. Both sample replicates were collected through the primed filter. Duplicate determinations were made in all cases.

Determinations of solubility at pH 2 and pH 7.4, and in FaSSIF and media were performed for 10 selected 2-AMT compounds (Table 3). Nine of the 2-AMTs exhibited poor solubility at pH 7.4, but good solubility in the media. IND44 showed lower solubility at pH 7.4 and in the media.

IND22 had higher solubility at both pH 2.0 and in FaSSIF. Among the others tested, IND44, IND46, and IND47 had lower solubility at both pH 2.0 and in FaSSIF. Some, like IND81 and IND85, had higher solubility at pH 2.0 but lower solubility in FaSSIF, whereas others, like IND24 and IND33, had higher solubility in FaSSIF but lower at pH 2.0. The pH-dependent solubility is most likely related to the pKa values for each compound.

Media Solubility

To evaluate media solubility, two protocols were followed. In the first, solutions were prepared as described above. After overnight equilibration, samples were centrifuged for 30 min, without filtration, and supernatant analyzed by HPLC/UV/VIS with reference to three standards (5, 100, 250 μM). In the second protocol, turbidimetric analysis, dilutions of the compound of interest were prepared in media with target concentrations of 5, 10, 20, 50, 100, 200 and 300 μM. Media without solute was included for background. These solutions were evaluated using a light scattering technique and a Nepheloskan instrument. A reading that was greater than or equal to 3 times background was considered the limit of solubility.

### R. Example 17

### Bidirectional MDCK-MDR¹ Cell Permeability

MDCK-MDR¹ cells were grown to confluence for 5-10 days on 1-μm filters in 24-well plates {Hilgers, 1990}. Aliquots of DMSO stocks were diluted into Hank's balanced salt solution (HBSS), pH 7.4, containing 25 mM +0.05% PS80 to give 10-μM solute concentration. The solute containing donor solutions were transferred to either the apical or basolateral chamber of the permeability diffusion apparatus. Receiver solutions consisted of HBSS (pH 7.4) containing 25 mM HEPES+0.05% PS80. Sequential samples of transported solute were taken at 20-min intervals using an automated liquid handling platform. The concentration of transported solute during each sampling interval was determined by HPLC/UV/MS. Permeability coefficients were calculated for each sampling interval. The average and standard deviation from the intervals are reported. Mass balance in the system was ascertained by comparing the sum of total transported solute and remaining donor solute with the starting mass of solute and is expressed as a percentage of donor solute at time zero. Significant deviations from 100% (generally less than 70%) suggest solute adsorption to the apparatus or monolayer, or chemical or metabolic instability during the course of the experiment. In the event of mass balance less than 70%, the cell monolayers were extracted with ACN and analyzed for the solute of interest. Determinations were conducted in duplicate.

Next, we evaluated whether any of the 2-AMTs are substrates for P-glycoprotein (P-gp), which is an efflux transporter in the blood-brain barrier. Permeability of the 10 select 2-AMTs was evaluated in MDCK-MDR¹ cells (Table 3). All 10 compounds tested exhibited acceptable permeability, with apparent permeability (Pₐₚₚ) values ranging from 3-50×10⁻⁶ cm/sec. Efflux ratios of the compounds tested ranged from 0.8 to 1.5, indicating that none are strong substrates for P-gp.

### S. Example 18

### Stability of 2-AMT in Liver Microsomes and Calculated Hepatic Extraction Ratio

Hepatic Microsome Stability.

Aliquots of DMSO solute stock were diluted into acetonitrile and then into assay buffer. Assay buffer was pH 7.4 phosphate buffered saline (PBS). Final experimental solute concentrations were 1 μM (0.6% acetonitrile, 0.01% DMSO). Commercially available rat human hepatic microsomes (approx 0.3 mg/ml final concentration) and NADPH (1 mM) with 4 mM UDPGA or PBS were added. The resulting mixture was incubated at 37° C. with aliquots removed at 0, 1, 10, 30 and 60 minutes and quenched with acetonitrile containing 2 μM carbamazepine (internal standard), centrifuged and supernatant analyzed by LCMS for remaining starting material. Duplicate incubations were run at each timepoint. Control incubations were conducted with Midazolam (T_{1/2}=11 min) and 4-Nitrophenol (T_{1/2}=20 min).

Hepatic microsomal stability assays were performed in two stages. In the first set of experiments, aliquots of the DMSO solute stocks were diluted into ACN and then into assay buffer (PBS, pH 7.4, ±0.05% PS80). Final experimental solute concentrations were 1 μM (0.6% ACN, 0.01% DMSO). Commercially available mouse, rat, or human hepatic microsomes (˜0.5 mg/ml final concentration), and NADPH (1 mM) or PBS were added. The resulting mixture was incubated at 37° C., and aliquots removed at 0, 1, 10, 30 and 60 min, then quenched with ACN containing 2 μM carbamazepine (internal standard). After centrifugation at 12,000×g for 10 min, the supernatant was analyzed by LC/MS for remaining starting material. Duplicate incubations were run for each time-point. The percentage of solute remaining at the end of the incubation was used to determine solute half-life.

A second set of experiments was conducted to include the positive control (IND54304), and test IND24 and IND81 in mouse, rat, dog (male and female) and human (mixed) hepatic microsomes to determine if there were gender effects. Stock solutions of 0.5 mM IND24, IND81, and IND54304 were prepared in DMSO. These were diluted 500-fold into 1 mL of microsomal incubation mixture to yield a final concentration of 1 μM. The incubation mixture was composed of 100 mM phosphate buffer, pH 7.4, and NADPH regenerating system (BD Biosciences NADPH Regenerating System Solutions A & B). This mixture was preincubated at 37° C. for 5 min in an Eppendorf Thermo mixer, and the reaction initiated by addition of 0.5 mg (25 μl of a 20 mg/mL solution) of liver microsomes. Aliquots (100 μl) were withdrawn at 0, 5, 15, 30, and 60 min, and added to 100 μl ACN containing internal standard. After centrifugation at 12,000×g for 10 min, the supernatants were analyzed by LC/MS/MS. The percentage of solute remaining at the end of the incubation was used to determine species-specific values of scaled intrinsic clearance, hepatic clearance, and predicted hepatic extraction based upon the calculations below {Obach, 1999 #9097}.

Initially, 10 2-AMT analogs were evaluated for hepatic microsomal stability using mouse, rat, and human liver microsomes. As can be seen from Table 4, the extraction ratios (ER) varied widely between the 2-AMT tested. IND24 appeared to be relatively stable in all 3 species tested, with a t_{1/2} of >60 min. A few of the 2-AMTs, like IND33, IND46, and IND47, had lower to moderate hepatic ER, whereas others like IND85 and IND52 exhibited higher hepatic ER in all species tested.

Preliminary results from in-vivo experiments in mice suggested that brain and plasma concentrations might differ for these compounds as a function of gender. Therefore, we evaluated hepatic microsomal stability in male and female mouse, rat, dog and human tissues for IND24, IND81, and IND54304. There appeared to be a gender difference in the metabolic stability of IND24 in dog microsomes and of IND81 in rat microsomes (Table 5). As observed previously, IND24 appeared to be stable in rat, mouse, and human liver microsomes, but exhibited a moderate hepatic ER in dog microsomes. The hepatic ER for IND81 was low in rat microsomes, moderate in human microsomes, and high in dog and mouse microsomes. Similarly, IND54304 exhibited a wide range of hepatic ER: low in human and rat, moderate in mouse, and high in dog.

### Intrinsic Clearance

$${Cl}_{int , microsomal} = \frac{0.693}{In vitro {T}_{1 \text{/} 2}} * \frac{ml incubation}{mg microsomes}$$

$$Scaled intrinsic clearance \text{:}$$

$${Cl}_{int , scaled} = \frac{0.693}{In vitro {T}_{1 \text{/} 2}} * \frac{ml incubation}{mg microsomes} * \frac{mg microsomes}{gm liver} * \frac{gm liver}{kg body weight}$$

for which mg microsomal protein/gm liver was 45 for all species, and gm liver/kg body weight were 32 for dog, 88 for mouse, 40 for rat and 25 for human.

### Hepatic Clearance

Clₕ=(Q*Cl_{int,scaled})/(Q+Cl_{int,scaled})

for which Q was hepatic blood flow; values of 90 ml/min/kg for mouse, 66 ml/min/kg for rat, and 21 ml/min/kg for human were used.

### Hepatic Extraction Ratio (ER)

ER=Clₕ/Q

for which ER estimates the amount of compound cleared during a single pass through the liver (Low ER<0.3, moderate ER 0.3-0.7, high ER>0.7).

### T. Example 19

### Plasma Protein and Brain Tissue Binding

Stock solutions of 1 mM and 0.1 mM of IND24, IND81 and warfarin (positive control) were prepared in DMSO and diluted 100-fold in dog, rat, mice and human plasma or in mouse brain homogenate (prepared by diluting brain samples from untreated FVB mice 5-fold with water and homogenizing using a Precellys 24 tissue homogenizer) to yield concentrations of 10 and 1 μM, respectively. The final DMSO concentration in plasma was 1%. In-vitro plasma protein binding was determined by using a rapid equilibrium dialysis (RED) device containing a dialysis membrane with a molecular weight cut-off of 8000 daltons. The experiments were run in duplicate (warfarin) or triplicate (IND24 and IND81). A 200-μl aliquot of the spiked plasma sample was placed in the sample chamber and 350 μl of Dulbecco's PBS, pH 7.4, was placed in the buffer chamber of the insert. The unit was covered with a sealing tape and incubated at 37° C. with shaking at 100 rpm for 4 h. Preliminary experiments were conducted with incubations up to 6 h to determine the time required to reach equilibrium. At the end of the incubation period, 50-μl aliquots each from the sample and buffer chambers were pipetted into separate microcentrifuge tubes. The buffer sample was diluted with 50 μl of appropriate blank plasma, and an equal volume of PBS, pH 7.4, was added to the plasma samples. For analysis, 150 μl of acetonitrile containing internal standard was added to 50-μl aliquots of the samples and centrifuged at 12,000×g for 10 min. The supernatants were analyzed by LC/MS/MS. Plasma protein binding was calculated as follows:

% Free=(Buffer concentration/Plasma concentration)×100%

% Bound=100%−% Free

The fraction unbound value in the diluted brain tissue (fuₘₑₐₛ) was calculated as above. This was converted to the undiluted fu value (fu_{brain}) using the following equation {Kalvass, 2002}.

$$fubrain = \frac{\frac{1}{d}}{( ( \frac{1}{fumeas} ) - 1 ) * \frac{1}{D}}$$

where D=dilution factor of the brain tissue.

Based on in-vivo results demonstrating that IND24 and IND81 had the best overall drug-like properties, we decided to advance these further and evaluated their binding in plasma and brain homogenates (Table 6). Binding was evaluated in dog, mouse, rat and human plasma, and mouse brain homogenates at concentrations of 1 and 10 μM using rapid equilibrium dialysis. Plasma protein binding for both compounds in plasma of all species tested ranged from 93-96%. Plasma protein binding values for warfarin, a control, were in good agreement with the published literature. Brain tissue binding was ˜90% for IND81 and 92-93% for IND24. These results indicate that IND81 and IND24 bound efficiently to plasma and brain tissue.

### U. Example 20

### Identification of Metabolites of IND24 and IND81

Preliminary Metabolite Detection

For metabolite detection, dog, rat, mouse, and human microsomal incubation samples with 1 μM IND24 or IND82 were scanned by multiple reaction monitoring (MRM) for potential hydroxylated metabolites. Samples were injected onto a BetaBasic C18 column and eluted with a gradient elution of mobile phase B (0.1% formic acid in ACN) from 5% to 95% over 30 min. For IND24-containing samples, the transitions monitored were 344→226 (parent); 360→226 and 360→242 (monohydroxylation); and 376→242 and 376→258 (dihydroxylation). For IND81-containing samples, the transitions monitored were 350→233 (parent); 366→233 and 366→249 (monohydroxylation); and 382→249 and 382→265 (dihydroxylation). Plasma and brain homogenate samples from FVB mice in single-dose (10 mg/kg) pharmacokinetic studies were similarly scanned to detect potential hydroxylated metabolites.

Metabolite Identification

Metabolites were separated on a Kinetex 100-Å column (2.6 μm, 100×2.1 mm, Phenomenex, Torrance, Calif.) at ambient temperature. The mobile phase consisted of 0.1% formic acid (Solvent A) and acetonitrile (Solvent B), and was delivered at 0.200 mL/min for 50 min. The initial composition of solvent B was maintained at 1% for 5 min and then increased in a linear manner as follows: 30% at 20 min; 50% at 25 min, maintained at 50% B for 3 min; and then increased to 90% at 40 min. Solvent B was maintained at 90% for up to 45 min and then decreased to 1% in the next 2 min. The column was allowed to equilibrate at 1% solvent B for 5 min prior to the next injection. The HPLC effluent going to the mass spectrometer was directed to waste through a divert valve for the initial 5 min after sample injection. Mass spectrometric analyses were performed on a ThermoFinnigan LTQ Orbitrap mass spectrometer (Thermo Scientific; Waltham Mass.), which was interfaced to an Agilent HP-1100 HPLC system (Agilent Technologies, Palo Alto, Calif.) and equipped with an electrospray ionization source (ESI). The parameters for the ESI source were: capillary temperature 325° C.; source voltage 3.5 kV; source current 100 μA; capillary voltage 33.0 V. The mass spectrometer was operated in a positive ion mode with data-dependent scanning

The Orbitrap mass analyzer was calibrated according to the manufacturer's directions using a mixture of caffeine, Met-Arg-Phe-Ala (MRFA) peptide and Ultramark 1621. The parent compounds and their metabolites were detected by full scan mass analysis from m/z 150-1000 at a resolving power of 60,000 [at m/z 400, full width at half maximum (FWHM); 1-s acquisition] with data-dependent MS/MS analyses triggered by the most abundant ion. This was followed by MS³ of the most abundant product ion. The resolving power used for multiple stage mass analysis was the same as the full-scan mass analysis. The CID was conducted with an isolation width of 3 Da, normalized collision energy of 35 for MS/MS and MS³, activation q of 0.25 and an activation time of 30 ms. Default automatic gain control (AGC) target ion values were used for MS, MS/MS and MS³ analyses. The data obtained was analyzed using Xcalibur v2.1 software (ThermoScientific; Waltham Mass.). Four-decimal monoisotopic masses of the parent compounds and their oxidative metabolites calculated using ChemBioDraw Ultra software version 11.0 (CambridgeSoft; Cambridge, Mass.) were used to interpret further the fragment ions.

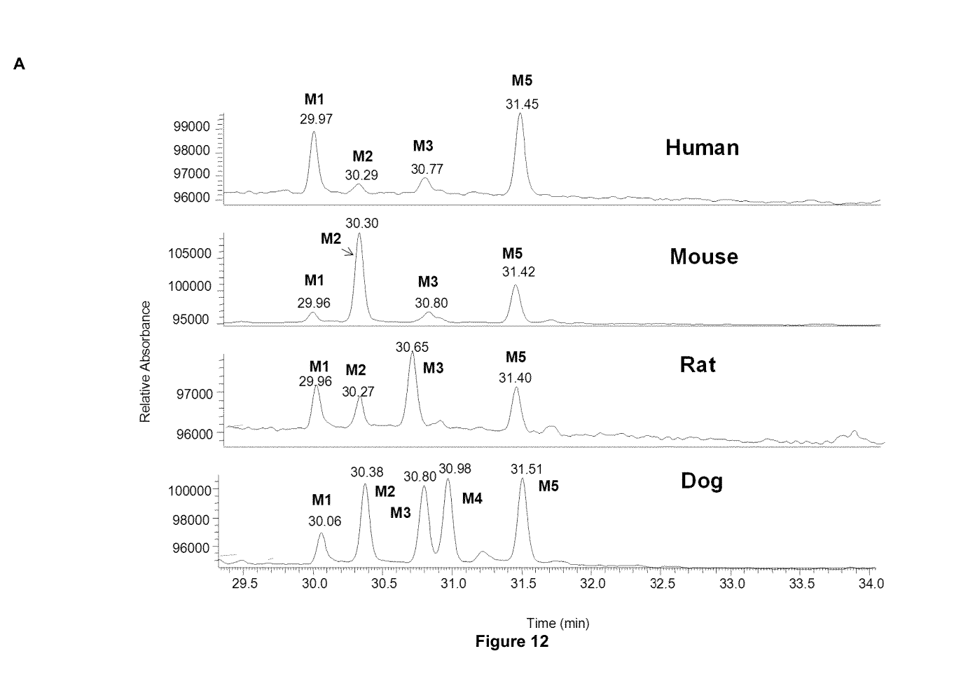

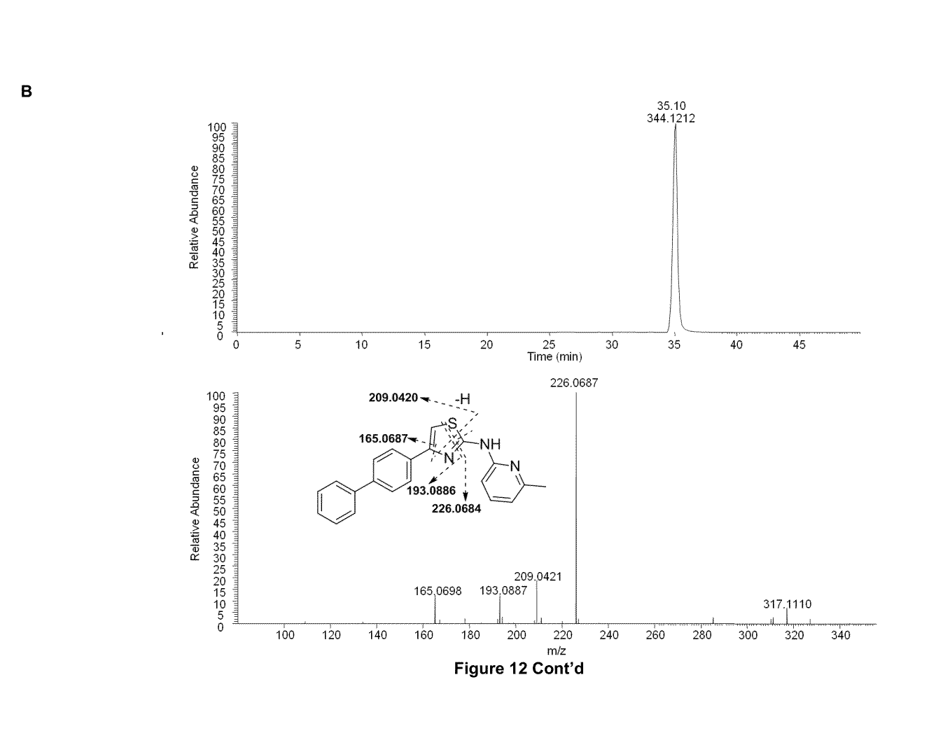

In preliminary metabolite detection using dog, rat, mouse, and human microsomes, and 1 μM of IND24 and IND81, we observed several mono- and di-hydroxylated metabolites in all samples. These IND24 and IND81 metabolites were also found in plasma and brain samples from FVB mice after a single oral dose of 10 mg/kg. To identify the metabolic profile for IND24 and IND81, we incubated the compounds (10 μM) with human, mouse, rat, and dog liver microsomes, then scanned samples by LC/MS/MS. The structures of the metabolites were characterized using an Orbitrap mass spectrometer and by comparison with the mass spectrum of the parent compound. For IND24, metabolites M1-M3 and M5 were observed in all of the microsomal assays while M4 was only observed in the dog liver microsomal incubation (FIG. 12A). Unchanged compound IND24 eluted at 35.1 min and gave a protonated molecular ion (MH⁺) at m/z 344.1212 (FIG. 12B). The mass spectrum of MH⁺ ion at m/z 344.1212 gave fragment ions at m/z 226.0687, 209.0421, 193.0886 and 165.0698 (FIG. 12B, Table 7). These fragment ions were most likely formed via cleavage of the thiazole moiety because their masses were similar to the calculated masses (Table 14 and FIG. 12B). The peaks at 30 (M1), 30.3 (M2), 30.8 (M3), 31 (M4) and 31.5 (M5) min gave a MH⁺ ion at m/z 360, an addition of 16 amu to 344, suggesting hydroxylation of the parent. The mass spectrum of M1 at m/z 360.1162 showed a major fragment ion at m/z 342.1060 in the MS² mass spectrum (Table 14). Further fragmentation of the ion m/z 342.1060 in a data-dependent manner (MS³) resulted in major ions at m/z 315.0951, 309.1262 and 161.0167. The ion at m/z 342.1060 resulted from loss of a water molecule from m/z 360.1162; a loss of a water molecule suggests that either the sulfur atom of the thiazole moiety or the methyl group on the pyridine ring was hydroxylated. The exact masses of the fragment ions at m/z 315, 309 and 161 were also in good agreement with the calculated exact masses (Table 14). It is well known that the S-oxides formed via oxidation of thiophene rings are generally unstable {Ha-Duong, 2001}. The same probably applies to the thiazolyl S-oxides. Hence, the site of hydroxylation in metabolite M1 is proposed to be the methyl group on the pyridine ring (FIG. 14).

For M2, M3, and M4 of IND24, the mass spectrum of MH⁺ at m/z 360.1158 resulted in fragment ions at m/z 242.0634 and 225.0369, and m/z 209.0835 and 181.0647 in the MS² and MS³ spectra, respectively. An addition of 16 amu indicated an insertion of oxygen into the molecule, but the lack of an ion resulting from loss of a water molecule in the mass spectra suggested that either the biphenyl ring or the pyridine ring of the 2-methylaminopyridine moiety was modified. Modification of the methylpyridine group was ruled out by the ions at m/z 242, 225, 209 and 181, which showed an addition of 16 amu to the masses of the fragment ions observed in the mass spectrum of the parent compound. The observed exact masses of the ions were also in good agreement with the calculated exact masses for the projected fragment ions (Table 14). This observation suggests modification of the biphenyl ring in all metabolites (FIG. 14). Although the spectra of the metabolites indicated modification of the biphenyl ring, the exact position of hydroxylation on this moiety could not be determined. Because the mass spectra of M2, M3, and M4 were similar, the metabolites are assumed to be regioisomers. The mass spectrum of M5 at m/z 360.1160 gave fragment ions at m/z 226.0686, 209.0421, 193.0886 and 165.0698 that were consistent with those observed in the mass spectrum of the parent compound. Similarity between the fragment ions from the spectra of M5 and the parent IND24, and the lack of loss of a water molecule for M5, suggest hydroxylation of the pyridine ring of the methylpyridine moiety (FIG. 14).

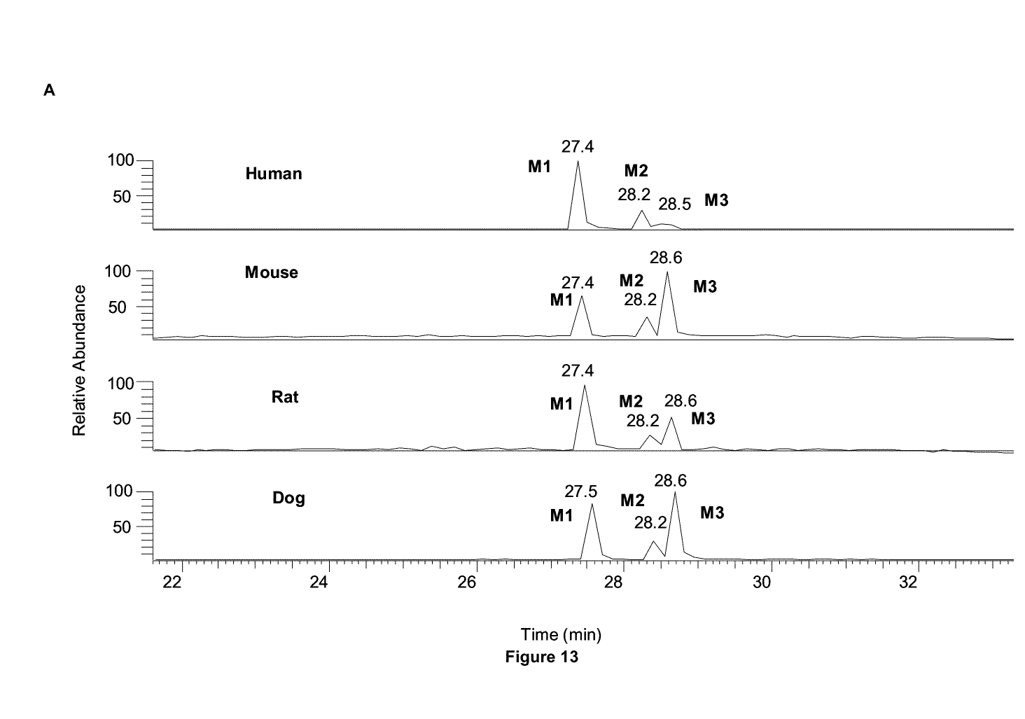

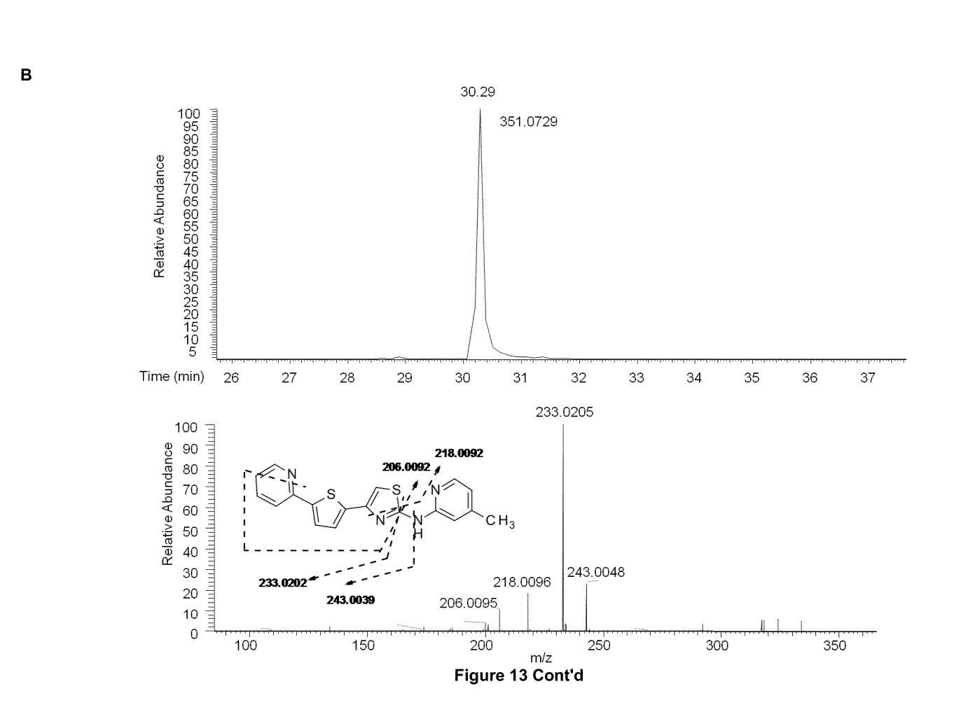

For IND81, metabolites M1-M3 were observed in microsomes of all species evaluated (FIG. 2A). Unchanged compound IND81 eluted at 30.3 min and gave a MH⁺ ion at m/z 351.0729 (FIG. 13B). The mass spectrum of MH⁺ ion at m/z 351.0729 gave fragment ions at m/z 243.0048, 233.0205, 218.0096 and 206.0095 (FIG. 13B, Table 14). The ion at m/z 243.0048 was a result of the cleavage of the bond between the amino group of the aminomethylpyridine ring and the thiazole ring (FIG. 14B, fragment ion a, Table 14). Other fragment ions were most likely generated via cleavage of the thiazole moiety, as their observed masses were similar to the calculated masses of these fragment ions. The peaks at 27.4 (M1), 28.2 (M2), and 28.5 (M3) min gave a molecular ion at m/z 367, an addition of 16 amu to m/z 351, suggesting hydroxylation of the parent IND81. The mass spectra of M1 and M3 at m/z 367.0680 were similar and showed fragment ions at m/z 258.9997, 249.0154, 234.0042 and 222.0041 (Table 14). All fragment ions indicated an addition of 16 amu to the masses of the fragment ions observed in the mass spectrum of the parent compound. The observed exact masses of the ions were also in good agreement with the calculated exact masses for the projected fragment ions (Table 14). This similarity suggests that the thienylpyridine ring of IND81 is the site of modification (FIG. 14). However, the exact site of oxidation could not be elucidated from this spectral information. Because the mass spectra of M1 and M3 were similar, the metabolites are assumed to be regioisomers. The mass spectrum of M2 at m/z 367.0679 gave fragment ions at m/z 243.0048, 233.0204, 218.0095, 206.0094, and 349.0576 (Table 14). While the first four ions were consistent with those observed in the mass spectrum of the parent compound, the ion at m/z 349 indicated a loss of water from the molecule. These data suggest that of the parent suggested that the methyl group on the pyridine ring of IND81 is the site of oxidation (FIG. 3).

### V. Example 21

### Human P450 Isoforms Involved in Metabolism of IND24 and IND81

Cytochrome P450 phenotyping

IND24 and IND81 were incubated in pooled human liver microsomes (HLMs; 0.5 mg/mL microsomal protein) in the presence or absence of selective inhibitors of P450s, specifically 10 μM furafylline with 15-min preincubation (CYP1A2), 5 μM sulfaphenazole (CYP2C9), 5 μM (+)-N-3-benzylnirvanol (CYP2C19), 1 μM quinidine (CYP2D6), and 1 μM ketoconazole (CYP3A4). IND24 and IND81 concentration used was 1 μM and the final organic solvent of the incubation mixture was less than 0.1%. Duplicate incubations were performed and the reaction stopped after 60 min by the addition of 0.1% formic acid in ACN containing internal standard. After vortexing and centrifugation at 12,000×g for 10 min, the supernatant was analyzed by LC/MS/MS.

Involvement in the metabolism of a specific CYP enzyme was estimated based on the following equations:

% Disappearance=[(TC-NADPH TC+NADPH)/TC-NADPH]×100

Δ Disappearance=(% Disappearance of TC in the presence of NADPH−% Disappearance of TC in the presence of NADPH and chemical inhibitor)

for which TC is the test compound.

To determine whether the P450 isoform is involved in the metabolism of IND24 and IND81, different P450 chemical inhibitors were coincubated with the 2-AMTs in HLMs. As has been observed previously, there was very little metabolism of IND24 (1 μM) in the HLM incubations, either in the presence or absence of any CYP inhibitors (Table 15). Therefore, the role of any specific P450 isozymes in the metabolism of IND24 could not be determined. In contrast, IND81 was metabolized in HLM with relatively high turnover (Table 15). Based on the change in percentage disappearance, the P450 isozymes most likely to be involved in the metabolism of IND81 are CYP1A2, CYP2D6 and CYP3A4; CYP2C19 might be involved to a smaller extent and CYP2C9 is unlikely to be involved.

### W. Example 22

### In-Vivo Studies

Single-Dose Pharmacokinetic Studies

Two-hundred thirty-five 2-AMTs were synthesized and tested for antiprion potency in dose-titration EC₅₀ studies using an ELISA-based assay. Of these, 34 2-AMT compounds were selected for testing in single-dose oral pharmacokinetic (PK) studies, based on potency, structural diversity and physicochemical parameters, including solubility, in some cases, stability in (mouse, rat, dog, and human) liver microsomes, and permeability. Twenty-seven compounds were evaluated at a single oral dose of 40 mg/kg; seven 2-AMTs were administered at 10 mg/kg. Six 2-AMTs were evaluated at both doses. The lower dose was chosen in the second round to provide a linear range.

Pharmacokinetic protocols employing mice were reviewed and approved by the UCSF Institutional Animal Care and Use Committee (IACUC). Female FVB mice, weighing approximately 25 g, were used for all in vivo pharmacokinetic studies. Mice were housed with free access to food and water, and were maintained on 12-h light/dark cycles for 1 week before dosing studies were initiated.

For the single-dose of 40 mg/kg, compounds were dissolved in a formulation containing 5% propylene glycol, 35% α-tocopheryl polyethylene glycol 1000 succinate (TPGS) and 70% polyethylene glycol 400 (PEG400), and administered by oral gavage. Two animals per time point were used. At specified time points after dosing (0.5, 1, 2, 4, 6, and 24 h), animals were euthanized by CO₂, and blood for plasma (by cardiac puncture) and brain samples were collected. The heparinized blood samples were centrifuged to obtain plasma. Brain samples were weighed, diluted 10-fold with water, and then homogenized using a Precellys 24 tissue homogenizer. The brain and plasma samples were flash-frozen on dry ice and then stored at −80° C. until analysis. For the single-dose administration at 10 mg/kg, compounds were dissolved in a formulation containing 20% propylene glycol, 5% ethanol, 5% labrosol, and 70% PEG400, and administered by oral gavage. Two animals per time point were used. At specified time points after dosing (0.25, 0.5, 1, 2, 4, 6, and 24 h), animals were euthanized by CO₂, and blood for plasma (by cardiac puncture) and brain samples were collected. The heparinized blood samples were centrifuged to obtain plasma. Brain samples were weighed, diluted five-fold with water, and then homogenized using a Precellys 24 tissue homogenizer. The brain and plasma samples were flash-frozen on dry ice and then stored at −80° C. until analysis.

IND24, IND81, and IND54304 were evaluated by single-dose pharmacokinetic studies administered at 1 mg/kg by intravenous (IV) injection. IND 81 and IND54304 were dissolved in either 10% DMSO in PEG 400/water (1:1) or 10% DMSO; IND24 was dissolved in 10% ethanol in PEG 400/water (1:1). Compounds were administered through tail vein injection. Two animals per time point were used. At specified time points after dosing (5 min, 0.25, 0.5, 1, 2, 4, and 6 h), animals were euthanized by CO₂. Blood and brain samples were obtained and processed as described above for the oral dosing at 40 mg/kg.

To determine the pharmacokinetics of the 2-AMTs, we administered all 34 2-AMTs in single oral doses to FVB mice. Doses were 40 mg/kg for 27 compounds and 10 mg/kg for 12 compounds; IND22, IND24, IND46, IND81, and IND85 were administered in both dosages (FIGS. 15 and 16). Brain exposure (AUCᵢₐₛₜ values) spanned four orders of magnitude across the 27 2-AMT analogs examined at 40 mg/kg (FIG. 15A). IND48 and IND49 had the lowest AUCₗₐₛₜ values; this observation is not surprising because they were the only two compounds examined that had more than one hydrogen bond donor. For the 12 analogs at 10 mg/kg doses, the range of AUCₗₐₛₜ values was less broad (FIG. 15B). Both IND24 and IND81 showed very good brain exposures, with brain:plasma AUCᵢₐₛₜ ratios of 2.6 and 5.5, respectively, compared to a ratio of 0.5 for IND54304 (Table 16). Comparison of the calculated PSA values (Tables 8 and 9) to the observed AUCₗₐₛₜ values (FIG. 15) showed only a modest correlation. We found no apparent correlation between molecular weight and brain exposure.

At both doses studied, the majority of 2-AMT analogs had maximal brain concentrations (Cₘₐₓ values) that exceed their in-vitro EC₅₀ values (ratios >1; FIG. 16). For 8 analogs at the 40 mg/kg dose, including IND24 and IND81, the Cₘₐₓ/EC₅₀ ratio exceeded 10 (FIG. 16A).

In addition to the oral doses, IND24, IND81 and IND54304 were administered at 1 mg/kg to female FVB mice by the IV route. The half-life of IND24 was 2 h, which was 2× and 10× longer than that of IND81 and IND54304, respectively (Table 16). IND24 also had higher oral bioavailability (40%) compared to 25-27% for the other 2 compounds.

### X. Example 23

### Multidose Pharmacokinetic Studies

Ten compounds were selected from among the 34 2-AMTs for three-day multiple-dose PK studies at PO doses ranging from 40-210 mg/kg. Selection was based on diversity in potency, chemical structure, and a range of physicochemical properties. We used a liquid diet to facilitate easy drug administration, as well as serve as the daily source of all water and food. This approach was chosen because the mouse bioassay studies that would be used to assess drug effects on survival would be expected to run for 110 to 400 days, or more; gavage dosing for such a long period is not feasible and more stressful to mice. For the multiple-dose PK studies, 4.5 g of the 2-AMT analogs was added to 15 mL of pure PEG400, vortexed and sonicated to ensure dissolution, then stored at −4° C. until needed. This highly concentrated PEG400 solution was subsequently diluted to final dosing concentrations and added to the rodent liquid diet, cocoa (taste-masking agent), and water.

Wild-type FVB mice weigh ˜25 g and typically drink 20 mL of liquid diet per day, allowing an estimate of daily drug consumption. A single dosing cohort consisted of three mice in a shared cage and liquid diet was provided at the start of the study in sufficient volume (˜200 mL) to last for the entire three-day period. At the end of the dosing period, animals were euthanized by CO₂, followed by collection of plasma (cardiac puncture) and removal of whole brain. Samples were processed and stored as described above for the 10 mg/kg single-dose studies. IND24, IND81, and IND22 were also dosed at 210 mg/kg/day for 14 days to evaluate drug tolerance; methods of administration and sample collection were as described above.

Plasma and brain homogenate samples were extracted using a protein-precipitation method and analyzed by specific LC/MS/MS methods developed for each compound dosed in vivo. The analytical method accuracy and precision were monitored by analyzing quality control (QC) samples that were prepared by the same methods as the plasma or brain homogenate samples. The brain and plasma concentration data were used to calculate the maximal concentration (Cₘₐₓ), area under the concentration-time curve (AUCₗₐₛₜ), and absolute bioavailability (% F) by noncompartmental analysis with sparse sampling performed using Phoenix WinNonlin 6.1 software (Pharsight, Mountain View, Calif.).

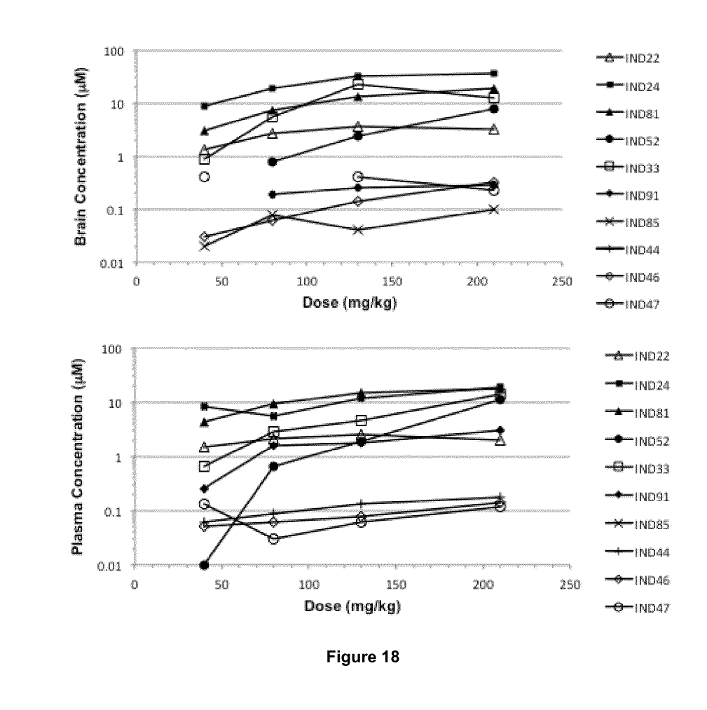

From the single-dose studies, 10 2-AMT analogs were selected for multidose pharmacokinetic studies. Compounds were administered at different doses (40, 80, 130, and 210 mg/kg/day) to FVB mice for 3 days in a liquid diet, then brain and plasma concentrations measured (FIG. 17). IND24 and IND81 were also administered at doses of 25, 75 and 125 mg/kg (FIG. 18). IND24, IND81 and IND33 achieved the highest concentrations in both brain and plasma for doses >40 mg/kg. For IND24, both brain and plasma concentrations appeared to reach a plateau at 125-130 mg/kg, with not much increase when the dose was raised to 210 mg/kg. IND81 showed linear increases in brain and plasma concentrations with increasing doses. IND33 showed a dose-dependent increase in plasma, but peaked at ˜30 μM with the 130 mg/kg dose in brain. IND52 had no measurable brain concentrations at a dose of 40 mg/kg, while exposure was comparable at all other doses. IND22 showed comparable brain and plasma concentrations at all doses. The brain and plasma concentrations of 5 analogs (IND85, IND44, IND46, IND47, IND91) were lower (≦1 μM).

Additionally, the pharmacokinetics of IND54304 was evaluated to determine the suitable dose for use as a positive control in future in-vivo drug efficacy studies. Doses of 25, 50, 100, and 150 mg/kg were administered to FVB mice for 3 days in a liquid rodent diet, then brain and plasma concentrations measured (FIG. 17C). All doses were tolerated, with no animals showing lethal toxicity. Doses of ≧150 mg/kg/day for 8 days resulted in 2/4 animals dying (data not shown). A dose of 100 mg/kg/day resulted in brain concentrations ˜10 μM (FIG. 17C), which is ˜20× the EC₅₀ value of 0.4 μM in ScN2a-c13 cells.

All of the multiple-day dosing studies previously described used a formulation that contained 1.25% (v/v) PEG400 in liquid diet. Male FVB did not appear to tolerate this amount of PEG400 in the formulation after long periods of dosing. To evaluate whether the amount of PEG400 in the final formulation could be reduced while maintaining high levels of IND24 in the brain, female FVB mice were dosed with 75 mg/kg IND24 in formulations containing 0.125-1.25% (v/v) PEG400. Brain concentrations of IND24 remained high (>8 μM, >6× the EC₅₀ value), even when the amount of PEG400 in the formulation was reduced 10-fold to 0.125% v/v (FIG. 19).

TABLE 1
Source and number of compounds tested in each HTS assay.

| Assay | Chembridge-1^{a} | Chembridge-2^{b} | SPECS^{c} | Total |
|---|---|---|---|---|
| PrP^{Sc} (dividing) | 22,703 | —^{d} | 30,127 | 52,830 |
| PrP^{Sc} (non-dividing) | 19,327 | —^{d} | 30,103 | 49,430 |
| PrP^{C} (IMR32) | 23,858 | 20,000^{e} | —^{d} | 43,858 |
| PrP^{C} (T98G) | 23,778 | 20,800^{e} | —^{d} | 44,578 |

^{a}Chembridge-1 (ChB-1) total library = 23,861 compounds
^{b}Chembridge-2 (ChB-2) total library = 39,840 compounds
^{c}SPECS total library = 30,256 compounds
^{d}None tested because compounds not available
^{e}Only half of the ChB-2 library was chosen for inclusion in the PrP^{C} assays

TABLE 2
Representative structures for 10 scaffolds identified in PrP^{C} assays.
Seven scaffolds were found for both IMR32 and T98G cells; three scaffolds were unique.

| | Murcko Fragment | | |
|---|---|---|---|
| | | IND-0082865 | |
| Amide | 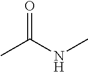 | MW: 316.4 % Inhibition: SPC (IMR32): 60 Calcein: 37 SPC (T98G): 51 Calcein: 17 | 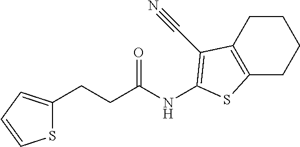 [SMILES: N#Cc1c(NC(=O)CCc2cccs2)sc2c1CCCC2] |
| | | IND-0017998 | |
| Fused Indole | 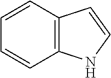 | MW: 324.8 % Inhibition: SPC (IMR32): 73 Calcein: 7 SPC (T98G): 74 Calcein: −19 | 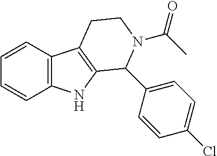 [SMILES: CC(=O)N1CCc2c([nH]c3ccccc23)C1c1ccc(Cl)cc1] |
| | | IND-0022317 | |
| AMT | 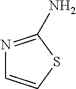 | MW: 267.4 % Inhibition: SPC (IMR32): 87 Calcein: 35 SPC (T98G): 65 Calcein: 5 | 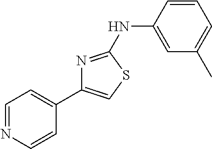 [SMILES: Cc1cccc(Nc2nc(-c3ccncc3)cs2)c1] |
| | | IND-0021750 | |
| Benzoylpiperazine | 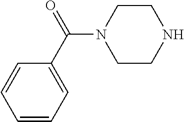 [SMILES: O=C(c1ccccc1)N1CCNCC1] | MW: 330.8 % Inhibition: SPC (IMR32): 44 Calcein: 25 SPC (T98G): 52 Calcein: 21 | 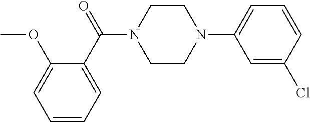 [SMILES: COc1ccccc1C(=O)N1CCN(c2cccc(Cl)c2)CC1] |
| | | IND-0020681 | |
| Chromene | 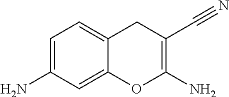 [SMILES: N#CC1=C(N)Oc2cc(N)ccc2C1] | MW: 319.4 % Inhibition: SPC (IMR32): 48 Calcein: 39 SPC (T98G): 43 Calcein: 21 | 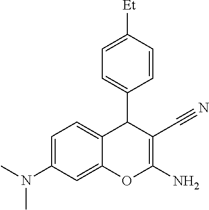 [SMILES: CCc1ccc(C2C(C#N)=C(N)Oc3cc(N(C)C)ccc32)cc1] |
| | | IND-0058521 | |
| Quinoline | 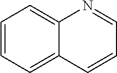 | MW: 322.4 % Inhibition: SPC (IMR32): 56 Calcein: 17 SPC (T98G): 49 Calcein: 3 | 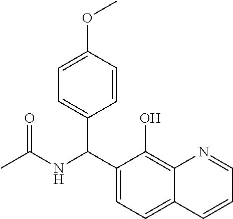 [SMILES: COc1ccc(C(NC(C)=O)c2ccc3cccnc3c2O)cc1] |
| | | IND-0014135 | |
| Sulfonamide | 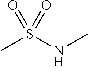 | MW: 289.4 % Inhibition SPC (IMR32): 55 Calcein: 4 SPC (T98G): 39 Calcein: 9 | 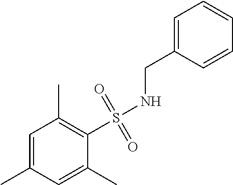 [SMILES: Cc1cc(C)c(S(=O)(=O)NCc2ccccc2)c(C)c1] |
| | | IND-0003070 | |
| Ethanolamine | 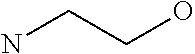 | MW: 285.4 % Inhibition: SPC (IMR32): 94 Calcein: 34 SPC (T98G): −55 Calcein: −13 | 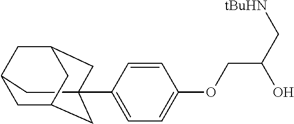 |
| | | IND-0081396 | |
| PyrimidineAmide | 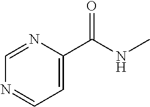 | MW: 339.8 % Inhibition: SPC (IMR32): 91 Calcein: 15 SPC (T98G): −5 Calcein: −8 | 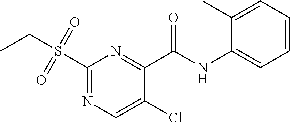 [SMILES: CCS(=O)(=O)c1ncc(Cl)c(C(=O)Nc2ccccc2C)n1] |
| | | IND-0002925 | |
| Urea | 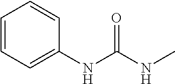 | MW: 342.3 % Inhibition: SPC (IMR32): 22 Calcein: 13 SPC (T98G): 62 Calcein: −10 | 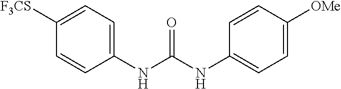 |
| | Murcko Fragment | | |
| | | IND-0061769 | |
| Amide | 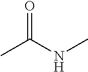 | MW: 304.2 % Inhibition: SPC (IMR32): 46 Calcein: 21 SPC (T98G): 63 Calcein: 38 | 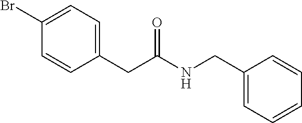 [SMILES: O=C(Cc1ccc(Br)cc1)NCc1ccccc1] |
| | | IND-0010878 | |
| Fused Indole | 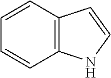 | MW: 318.4 % Inhibition: SPC (IMR32): 34 Calcein: 19 SPC (T98G): 64 Calcein: 25 | 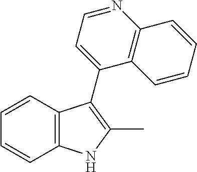 [SMILES: Cc1[nH]c2ccccc2c1-c1ccnc2ccccc12] |
| | | IND-0009688 | |
| AMT | 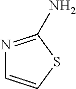 | MW: 288.8 % Inhibition: SPC (IMR32): 66 Calcein: 38 SPC (T98G): 31 Calcein: 8 | 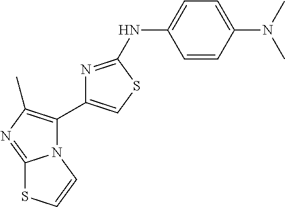 [SMILES: Cc1nc2sccn2c1-c1csc(Nc2ccc(N(C)C)cc2)n1] |
| | | IND-0023025 | |
| Benzoylpiperazine | 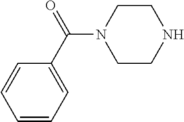 [SMILES: O=C(c1ccccc1)N1CCNCC1] | MW: 338.4 % Inhibition: SPC (IMR32): 34 Calcein: 15 SPC (T98G): 34 Calcein: 26 | 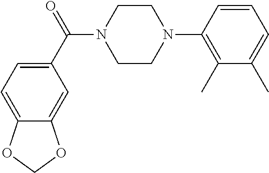 [SMILES: Cc1cccc(N2CCN(C(=O)c3ccc4c(c3)OCO4)CC2)c1C] |
| | | IND-0030883 | |
| Chromene | 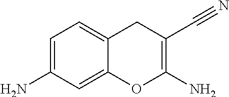 [SMILES: N#CC1=C(N)Oc2cc(N)ccc2C1] | MW: 331.3 % Inhibition: SPC (IMR32): 48 Calcein: 27 SPC (T98G): 42 Calcein: 15 | 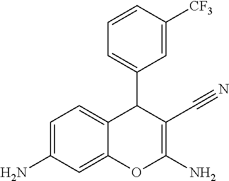 [SMILES: N#CC1=C(N)Oc2cc(N)ccc2C1c1cccc(C(F)(F)F)c1] |
| | | IND-0086056 | |
| Quinoline | 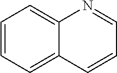 | MW: 306.4 % Inhibition: SPC (IMR32): 45 Calcein: 18 SPC (T98G): 49 Calcein: 20 | 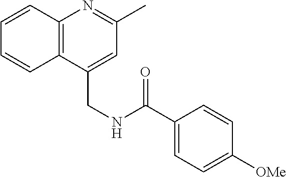 [SMILES: COc1ccc(C(=O)NCc2cc(C)nc3ccccc23)cc1] |
| | | IND-0007902 | |
| Sulfonamide | 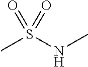 | MW: 291.4 % Inhibition: SPC (IMR32): 38 Calcein: 36 SPC (T98G): 51 Calcein: 19 | 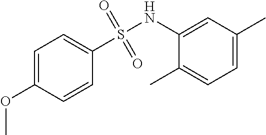 [SMILES: COc1ccc(S(=O)(=O)Nc2cc(C)ccc2C)cc1] |
| | | IND-0015066 | |
| Ethanolamine | 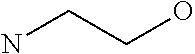 | MW: 309.2 % Inhibition: SPC (IMR32): 45 Calcein: 2 SPC (T98G): −11 Calcein: −27 | 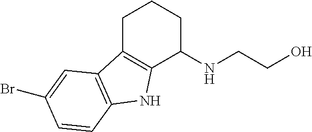 [SMILES: OCCNC1CCCc2c1[nH]c1ccc(Br)cc21] |
| | | IND-0081737 | |
| PyrimidineAmide | 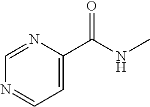 | MW: 343.8 % Inhibition: SPC (IMR32): 41 Calcein: 18 SPC (T98G): −10 Calcein: −4 | 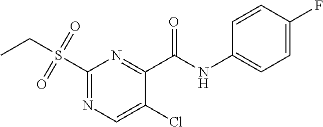 [SMILES: CCS(=O)(=O)c1ncc(Cl)c(C(=O)Nc2ccc(F)cc2)n1] |
| | | IND-0088907 | |
| Urea | 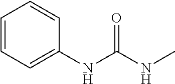 | MW: 256.3 % Inhibition: SPC (IMR32): 4 Calcein: 9 SPC (T98G): 46 Calcein: 27 | 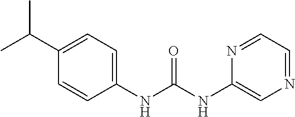 [SMILES: CC(C)c1ccc(NC(=O)Nc2cnccn2)cc1] |

TABLE 3
Distribution of EC₅₀ values for the 14 chemical scaffolds identified in dividing ScN2a-cl3 cells.

| | Murcko | Compounds | Compounds with | Compounds with |
|---|---|---|---|---|
| Scaffold Name | Fragment | tested (n) | EC₅₀: <1 μM (n) | EC₅₀: 1-10 μM (n) |
| Aminothiazole | 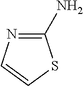 | 11 | 2 | 4 |
| Thiazole | 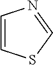 | 16 | 5 | 3 |
| Benzoxazole | 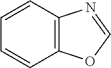 | 17 | 3 | 6 |
| Pyrazole | 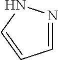 | 6 | 4 | 1 |
| Thienopyridine | 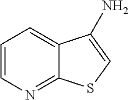 | 54 | 18 | 16 |
| Piperazine | 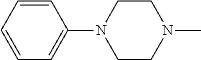 | 11 | 0 | 3 |
| Imidazopyridine | 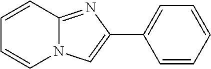 [SMILES: c1ccc(-c2cn3ccccc3n2)cc1] | 4 | 0 | 2 |
| Imidazothiazole | 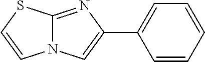 [SMILES: c1ccc(-c2cn3ccsc3n2)cc1] | 15 | 4 | 7 |
| Guanidine | 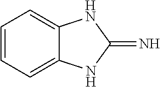 | 15 | 0 | 5 |
| Quinoline | 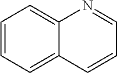 | 24 | 0 | 14 |
| Quinazoline | 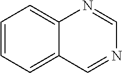 | 8 | 0 | 4 |
| Benzamide | 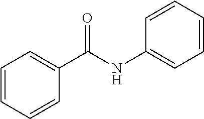 [SMILES: O=C(Nc1ccccc1)c1ccccc1] | 95 | 12 | 31 |
| Benzyl ether | 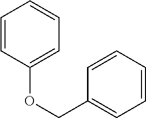 [SMILES: c1ccc(COc2ccccc2)cc1] | 9 | 0 | 4 |
| Stilbene | 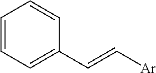 | 7 | 2 | 2 |

TABLE 4
The specific structures for each of the 14 scaffolds identified in dividing ScN2a-cl3 cells, with corresponding
EC₅₀ results from ELISA and Western immunoblots, LC₅₀ values from calcein assays, and Qikprop predictions of physicochemical properties.

| | Murcko Fragment | | | |
|---|---|---|---|---|
| | | IND-0030410 | | |
| AMT | 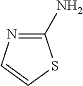 | MW: 320.4 EC50 (nM): ELISA: 317 Calcein: >10,000 Western: 194 | 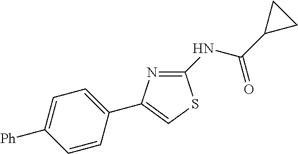 [SMILES: O=C(Nc1nc(-c2ccc(-c3ccccc3)cc2)cs1)C1CC1] | ClogP: 4.45 PSA: 47.91 HBD/HBA: 1/4 QPCaco-2: 2730.9 QPlogBBB: −0.122 |
| | | IND-0024350 | | |
| Thiazole | 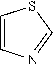 | MW: 277.3 EC50 (nM): ELISA: 321 Calcein: >10,000 Western: 828 | 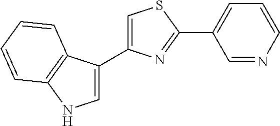 [SMILES: c1cncc(-c2nc(-c3c[nH]c4ccccc34)cs2)c1] | ClogP: 3.75 PSA: 37.5 HBD/HBA: 1/3 QPCaco-2: 2469.0 QPlogBBB: 0.081 |
| | | IND-0114202 | | |
| Benzoxazole | 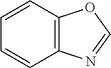 | MW: 450.5 EC50 (nM): ELISA: 57 Calcein: >10,000 Western: 2565 | 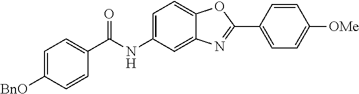 [SMILES: COc1ccc(-c2nc3cc(NC(=O)c4ccc(OCc5ccccc5)cc4)ccc3o2)cc1] | ClogP: 6.13 PSA: 71.7 HBD/HBA: 1/6 QPCaco-2: 2564.3 QPlogBBB: −0.538 |
| | | IND-0025807 | | |
| Pyrazole | 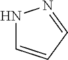 | MW: 296.3 EC50 (nM): ELISA: 167 Calcein: >10,000 Western: 95 | 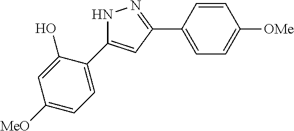 [SMILES: COc1ccc(-c2cc(-c3ccc(OC)cc3O)[nH]n2)cc1] | ClogP: 3.47 PSA: 63.8 HBD/HBA: 2/3 QPCaco-2: 1586.2 QPlogBBB: −0.415 |
| | | IND-0052025 | | |
| Thienopyridine | 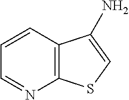 | MW: 391.4 EC50 (nM): ELISA: 57 Calcein: >10,000 Western: 61 | 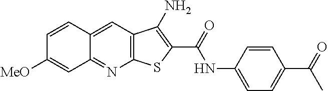 [SMILES: COc1ccc2cc3c(N)c(C(=O)Nc4ccc(C(C)=O)cc4)sc3nc2c1] | ClogP: 2.80 PSA: 109.9 HBD/HBA: 2/6 QPCaco-2: 72.0 QPlogBBB: −0.950 |
| | | IND-0018629 | | |
| Piperazine | 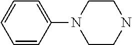 [SMILES: c1ccc(N2CCNCC2)cc1] | MW: 295.4 EC50 (nM): ELISA: 2943 Calcein: >10,000 Western: 886 | 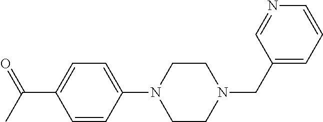 [SMILES: CC(=O)c1ccc(N2CCN(Cc3cccnc3)CC2)cc1] | ClogP: 2.09 PSA: 49.6 HBD/HBA: 0/7 QPCaco-2: 425.6 QPlogBBB: 0.014 |
| | | IND-0009419 | | |
| Imidazopyridine | 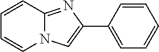 [SMILES: c1ccc(-c2cn3ccccc3n2)cc1] | MW: 236.3 EC50 (nM): ELISA: 709 Calcein: >10,000 Western: 265 | 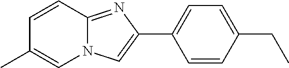 [SMILES: CCc1ccc(-c2cn3cc(C)ccc3n2)cc1] | ClogP: 4.53 PSA: 14.7 HBD/HBA: 0/2 QPCaco-2: 6882.3 QPlogBBB: 0.346 |
| | | IND-0009416 | | |
| Imidazothiazole | 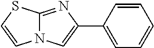 [SMILES: c1ccc(-c2cn3ccsc3n2)cc1] | MW: 242.3 EC50 (nM): ELISA: 1776 Calcein: >10,000 Western: 517 | 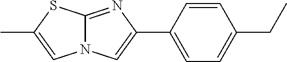 [SMILES: CCc1ccc(-c2cn3cc(C)sc3n2)cc1] | ClogP: 4.43 PSA: 14.7 HBD/HBA: 0/2 QPCaco-2: 6469.8 QPlogBBB: 0.438 |
| | | IND-0026326 | | |
| Guanidine | 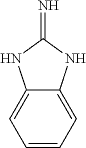 | MW: 299.8 EC50 (nM): ELISA: 5900 Calcein: >10,000 Western: 4967 | 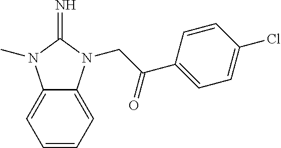 [SMILES: Cn1c(=N)n(CC(=O)c2ccc(Cl)cc2)c2ccccc21] | ClogP: 4.07 PSA: 53.8 HBD/HBA: 1/3 QPCaco-2: 1928.0 QPlogBBB: −0.21 |
| | | IND-0045193 | | |
| Quinoline | 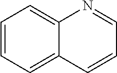 | MW: 352.9 EC50 (nM): ELISA: 1220 Calcein: >10,000 Western: 2073 | 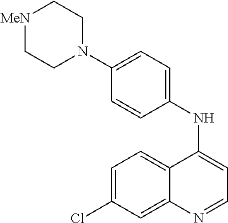 [SMILES: CN1CCN(c2ccc(Nc3ccnc4cc(Cl)ccc34)cc2)CC1] | ClogP: 4.36 PSA: 30.2 HBD/HBA: 1/5 QPCaco-2: 1187.2 QPlogBBB: 0.677 |
| | | IND-0034921 | | |
| Quinazoline | 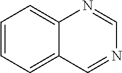 | MW: 326.8 EC50 (nM): ELISA: 3693 Calcein: >10,000 Western: 2009 | 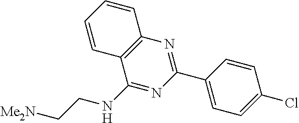 [SMILES: CN(C)CCNc1nc(-c2ccc(Cl)cc2)nc2ccccc12] | ClogP: 4.00 PSA: 33.4 HBD/HBA: 1/5 QPCaco-2: 1436.2 QPlogBBB: 0.595 |
| | | IND-0033145 | | |
| Benzamide | 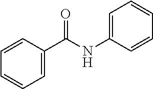 [SMILES: O=C(Nc1ccccc1)c1ccccc1] | MW: 324.4 EC50 (nM): ELISA: 412 Calcein: >10,000 Western: 161 | 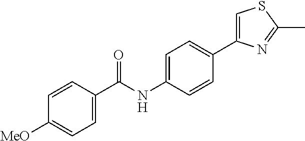 [SMILES: COc1ccc(C(=O)Nc2ccc(-c3csc(C)n3)cc2)cc1] | ClogP: 4.09 PSA: 54.9 HBD/HBA: 1/5 QPCaco-2: 2923.7 QPlogBBB: −0.063 |
| | | IND-0046380 | | |
| Benzyl ether | 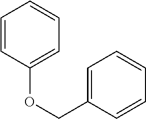 [SMILES: c1ccc(COc2ccccc2)cc1] | MW: 400.7 EC50 (nM): ELISA: 5080 Calcein: >10,000 Western: 2148 | 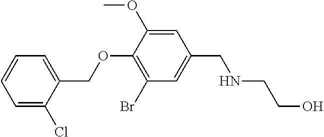 [SMILES: COc1cc(CNCCO)cc(Br)c1OCc1ccccc1Cl] | ClogP: 3.95 PSA: 49.7 HBD/HBA: 2/5 QPCaco-2: 557.1 QPlogBBB: −0.004 |
| | | IND-0047979 | | |
| Stilbene | 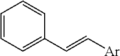 | MW: 223.3 EC50 (nM): ELISA: 1700 Calcein: >10,000 Western: 528 | 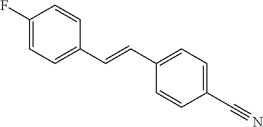 [SMILES: N#Cc1ccc(/C=C/c2ccc(F)cc2)cc1] | ClogP: 3.77 PSA: 25.8 HBD/HBA: 0/2 QPCaco-2: 2055.6 QPlogBBB: −0.229 |
| | Murcko Fragment | | | |
| | | IND-0030883 | | |
| AMT | 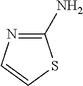 | MW: 294.4 EC50 (nM): ELISA: 913 Calcein: >10,000 Western: 570 | 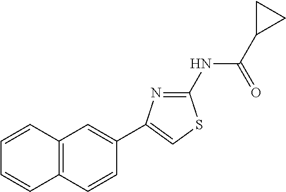 [SMILES: O=C(Nc1nc(-c2ccc3ccccc3c2)cs1)C1CC1] | ClogP: 3.79 PSA: 47.9 HBD/HBA: 1/4 QPCaco-2: 2728.5 QPlogBBB: −0.036 |
| | | IND-0024352 | | |
| Thiazole | 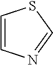 | MW: 291.4 EC50 (nM): ELISA: 2267 Calcein: >10,000 Western: 1698 | 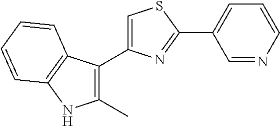 [SMILES: Cc1[nH]c2ccccc2c1-c1csc(-c2cccnc2)n1] | ClogP: 4.06 PSA: 35.8 HBD/HBA: 1/3 QPCaco-2: 2996.6 QPlogBBB: 0.163 |
| | | IND-0114138 | | |
| Benzoxazole | 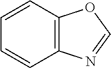 | MW: 388.4 EC50 (nM): ELISA: 65 Calcein: >10,000 Western: 134 | 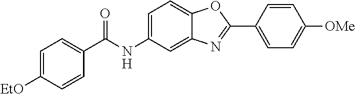 [SMILES: CCOc1ccc(C(=O)Nc2ccc3oc(-c4ccc(OC)cc4)nc3c2)cc1] | ClogP: 4.69 PSA: 72.4 HBD/HBA: 1/6 QPCaco-2: 2447.5 QPlogBBB: −0.456 |
| | | IND-0028801 | | |
| Pyrazole | 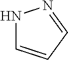 | MW: 280.3 EC50 (nM): ELISA: 245 Calcein: >10,000 Western: 117 | 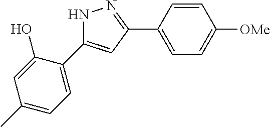 [SMILES: COc1ccc(-c2cc(-c3ccc(C)cc3O)[nH]n2)cc1] | ClogP: 3.71 PSA: 55.5 HBD/HBA: 2/3 QPCaco-2: 1586.8 QPlogBBB: −0.357 |
| | | IND-0052851 | | |
| Thienopyridine | 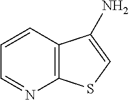 | MW: 295.3 EC50 (nM): ELISA: 962 Calcein: >10,000 Western: 518 | 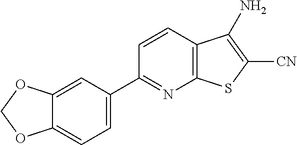 [SMILES: N#Cc1sc2nc(-c3ccc4c(c3)OCO4)ccc2c1N] | ClogP: 1.48 PSA: 81.6 HBD/HBA: 1.5/5 QPCaco-2: 131.5 QPlogBBB: −0.276 |
| | | IND-0019136 | | |
| Piperazine | 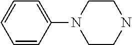 [SMILES: c1ccc(N2CCNCC2)cc1] | MW: 294.4 EC50 (nM): ELISA: 8681 Calcein: >10,000 Western: 615 | 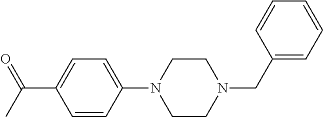 [SMILES: CC(=O)c1ccc(N2CCN(Cc3ccccc3)CC2)cc1] | ClogP: 3.16 PSA: 36.8 HBD/HBA: 0/5 QPCaco-2: 784.9 QPlogBBB: 0.269 |
| | | IND-0009417 | | |
| Imidazopyridine | 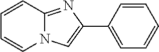 [SMILES: c1ccc(-c2cn3ccccc3n2)cc1] | MW: 222.3 EC50 (nM): ELISA: 9158 Calcein: >10,000 Western: 4715 | 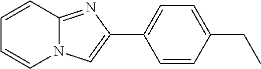 [SMILES: CCc1ccc(-c2cn3ccccc3n2)cc1] | ClogP: 4.26 PSA: 14.7 HBD/HBA: 0/2 QPCaco-2: 6882.6 QPlogBBB: 0.351 |
| | | IND-009413 | | |
| Imidazothiazole | 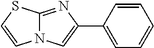 [SMILES: c1ccc(-c2cn3ccsc3n2)cc1] | MW: 250.2 EC50 (nM): ELISA: 6015 Calcein: >10,000 Western: 2490 | 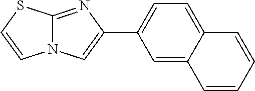 [SMILES: c1ccc2cc(-c3cn4ccsc4n3)ccc2c1] | ClogP: 4.28 PSA: 14.7 HBD/HBA: 0/2 QPCacQ-2: 6473.8 QPlogBBB: 0.523 |
| | | IND-0044723 | | |
| Guanidine | 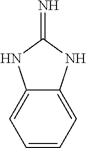 | MW: 344.2 EC50 (nM): ELISA: 1983 Calcein: >10,000 Western: 1453 | 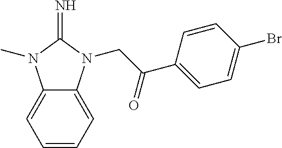 [SMILES: Cn1c(=N)n(CC(=O)c2ccc(Br)cc2)c2ccccc21] | ClogP: 4.14 PSA: 53.8 HBD/HBA: 1/3 QPCaco-2: 1928.1 QPlogBBB: −0.20 |
| | | IND-0044355 | | |
| Quinoline | 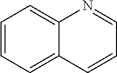 | MW: 340.4 EC50 (nM): ELISA: 2143 Calcein: >10,000 Western: 1833 | 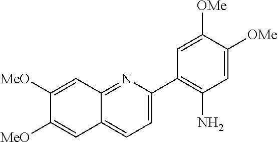 [SMILES: COc1cc(N)c(-c2ccc3cc(OC)c(OC)cc3n2)cc1OC] | Clog P: 3.72 PSA: 64.1 HBD/HBA: 1.5/5 QPCaco-2: 2957.7 QPlogBBB: −0.339 |
| | | IND-0035916 | | |
| Quinazoline | 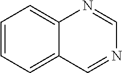 | MW: 266.3 EC50 (nM): ELISA: 4023 Calcein: >10,000 Western: 5987 | 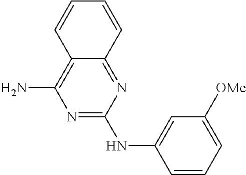 [SMILES: COc1cccc(Nc2nc(N)c3ccccc3n2)c1] | CIogP: 2.60 PSA: 63.9 HBD/HBA: 3/4 QPCaco-2: 1447.2 QPlogBBB: −0.499 |
| | | IND-0031751 | | |
| Benzamide | 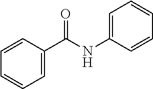 [SMILES: O=C(Nc1ccccc1)c1ccccc1] | MW: 307.3 EC50 (nM): ELISA: 156 Calcein: >10,000 Western: 42 | 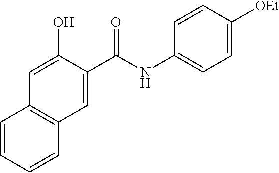 [SMILES: CCOc1ccc(NC(=O)c2cc3ccccc3cc2O)cc1] | ClogP: 4.38 PSA: 63.0 HBD/HBA: 1/3 QPCaco-2: 1544.2 QPlogBBB: −0.607 |
| | | IND-0033452 | | |
| Benzyl ether | 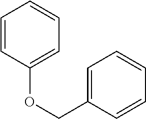 [SMILES: c1ccc(COc2ccccc2)cc1] | MW: 405.1 EC50 (nM): ELISA: 2850 Calcein: 27% Western: 1993 | 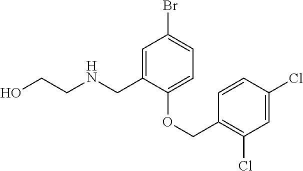 [SMILES: OCCNCc1cc(Br)ccc1OCc1ccc(Cl)cc1Cl] | ClogP: 4.24 PSA: 41.7 HBD/HBA: 2/4 QPCaco-2: 581.2 QPlogBBB: 0.3 |
| | | IND-0047971 | | |
| Stilbene | 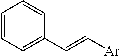 | MW: 228.2 EC50 (nM): ELISA: 2260 Calcein: >10,000 Western: 584 | 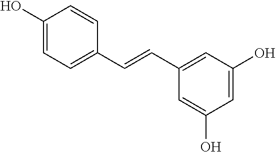 [SMILES: Oc1ccc(/C=C/c2cc(O)cc(O)c2)cc1] | ClogP: 2.01 PSA: 67.6 HBD/HBA: 3/2 QPCaco-2: 277.0 QPlogBBB: −0.129 |

(QPCaco-2 is in units of 10⁻⁶ cm/s and PSA is in units of angstroms.)

TABLE 5
Antiprion activity (EC₅₀) and
cytotoxicity (LC₅₀) for selected thienopyridine analogs.

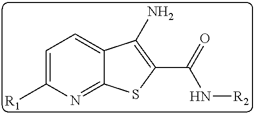

| | | | |
|---|---|---|---|
| Compound | R2 | EC₅₀ μM | LC₅₀ μM |

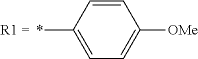

| | | | |
|---|---|---|---|
| IND-0035860 | 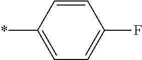 [SMILES: *c1ccc(F)cc1] | 0.057 | >10 |
| IND-0037769 | 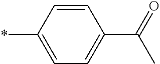 [SMILES: *c1ccc(C(C)=O)cc1] | 0.097 | >10 |
| IND-0035833 | 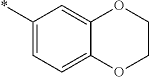 [SMILES: *c1ccc2c(c1)OCCO2] | >10 | >10 |
| IND-0040541 | 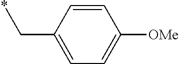 [SMILES: *Cc1ccc(OC)cc1] | >10 | >10 |
| IND-0037771 | 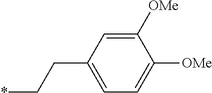 [SMILES: *CCc1ccc(OC)c(OC)c1] | 0.419 | >10 |

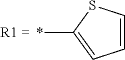

| | | | |
|---|---|---|---|
| IND-0024576 | 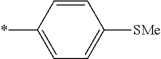 [SMILES: *c1ccc(SC)cc1] | 0.077 | >10 |
| IND-0024573 | 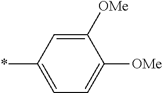 [SMILES: *c1ccc(OC)c(OC)c1] | 0.396 | >10 |
| IND-0024581 | 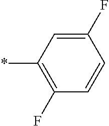 [SMILES: *c1cc(F)ccc1F] | 2.06 | >10 |
| IND-0042103 | 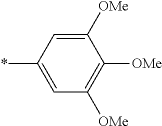 [SMILES: *c1cc(OC)c(OC)c(OC)c1] | >10 | >10 |

TABLE 6
Antiprion activity (EC₅₀) and cytotoxicity (LC₅₀) for selected thienopyridine analogs.

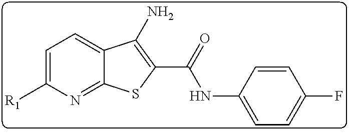

| Compound | R1 | EC₅₀ μM | LC₅₀ μM |
|---|---|---|---|
| IND-0035860 | 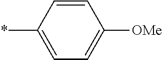 [SMILES: *c1ccc(OC)cc1] | 0.059 | >10 |
| IND-0044746 | 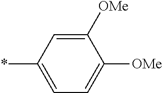 [SMILES: *c1ccc(OC)c(OC)c1] | 0.227 | >10 |
| IND-0042063 | 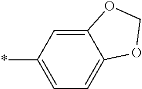 [SMILES: *c1ccc2c(c1)OCO2] | 0.613 | >10 |

| Compound | | EC₅₀ μM |
|---|---|---|
| IND-0024575 | 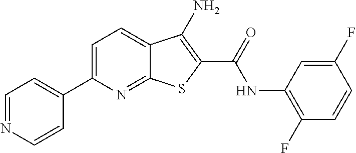 [SMILES: Nc1c(C(=O)Nc2cc(F)ccc2F)sc2nc(-c3ccncc3)ccc12] | 3.49 |
| IND-0052025 | 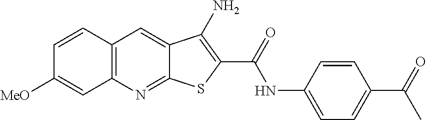 [SMILES: COc1ccc2cc3c(N)c(C(=O)Nc4ccc(C(C)=O)cc4)sc3nc2c1] | 0.074 |
| IND-0024609 | 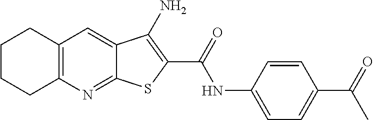 [SMILES: CC(=O)c1ccc(NC(=O)c2sc3nc4c(cc3c2N)CCCC4)cc1] | >10 |

TABLE 7
Mean ± SD of brain and plasma concentrations [area
under the curve values from first to last measureable time points
(AUCₗₐₛₜ)] for IND-0045193 and IND-0052851 after oral
administration of a single dose (10 mg/kg) of each to two
FVB mice. Chemical structures shown in Table 4.

| | | | Brain/Plasma AUCₗₐₛₜ |
|---|---|---|---|
| Compound | Matrix | AUCₗₐₛₜ ± SD (μM*h) | Ratio |
| IND-0045193 | Brain | 7.36 ± 0.78 | 4.72 |
| | Plasma | 1.56 ± 0.31 | |
| IND-0052851 | Brain | 2.12 ± 0.93 | 2.59 |
| | Plasma | 0.82 ± 0.31 | |

TABLE 8
AMT analogs synthesized and tested for potency, with calculated parameters.

| Compound | Structure | EC₅₀ (μM) | MW | tPSA | xlogP | HBA/HBD |
|---|---|---|---|---|---|---|
| IND7 | 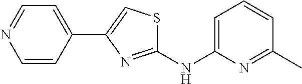 [SMILES: Cc1cccc(Nc2nc(-c3ccncc3)cs2)n1] | 1.00 | 268.3 | 50.7 | 3.05 | 4/1 |
| IND22 | 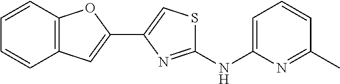 [SMILES: Cc1cccc(Nc2nc(-c3cc4ccccc4o3)cs2)n1] | 1.46 | 307.4 | 51.3 | 5.17 | 4/1 |
| IND24 | 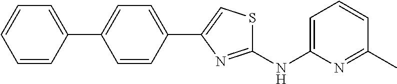 [SMILES: Cc1cccc(Nc2nc(-c3ccc(-c4ccccc4)cc3)cs2)n1] | 1.27 | 343.4 | 37.8 | 6.27 | 3/1 |
| IND29 | 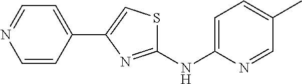 [SMILES: Cc1ccc(Nc2nc(-c3ccncc3)cs2)nc1] | 1.57 | 268.3 | 50.7 | 3.13 | 4/1 |
| IND26 | 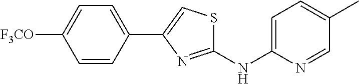 [SMILES: Cc1ccc(Nc2nc(-c3ccc(OC(F)(F)F)cc3)cs2)nc1] | >10.0 | 351.4 | 47.0 | 5.81 | 4/1 |
| IND33 | 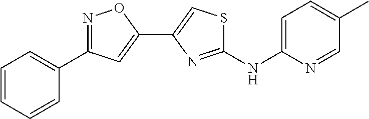 [SMILES: Cc1ccc(Nc2nc(-c3cc(-c4ccccc4)no3)cs2)nc1] | 0.94 | 334.4 | 64.1 | 4.29 | 5/1 |
| IND36 | 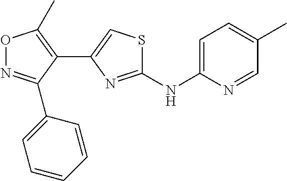 [SMILES: Cc1ccc(Nc2nc(-c3c(-c4ccccc4)noc3C)cs2)nc1] | >32.0 | 348.4 | 64.1 | 4.57 | 5/1 |
| IND38 | 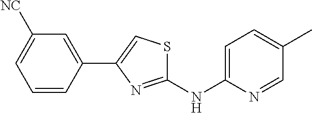 [SMILES: Cc1ccc(Nc2nc(-c3cccc(C#N)c3)cs2)nc1] | 2.29 | 292.4 | 61.6 | 4.16 | 4/1 |
| IND74 | 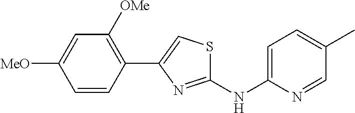 [SMILES: COc1ccc(-c2csc(Nc3ccc(C)cn3)n2)c(OC)c1] | 3.13 | 327.4 | 56.3 | 4.37 | 5/1 |
| IND76 | 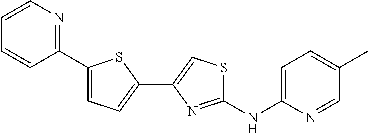 [SMILES: Cc1ccc(Nc2nc(-c3ccc(-c4ccccn4)s3)cs2)nc1] | 1.23 | 350.5 | 50.7 | 4.83 | 4/1 |
| IND2 | 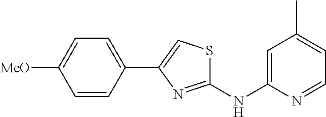 [SMILES: COc1ccc(-c2csc(Nc3cc(C)ccn3)n2)cc1] | 1.66 | 297.4 | 47.0 | 4.41 | 4/1 |
| IND78 | 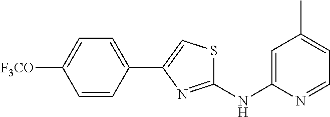 [SMILES: Cc1ccnc(Nc2nc(-c3ccc(OC(F)(F)F)cc3)cs2)c1] | >32.0 | 351.4 | 47.0 | 5.81 | 4/1 |
| IND48 | 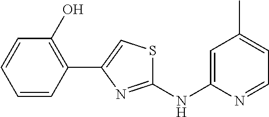 [SMILES: Cc1ccnc(Nc2nc(-c3ccccc3O)cs2)c1] | >10.0 | 283.4 | 58.0 | 4.07 | 4/2 |
| IND49 | 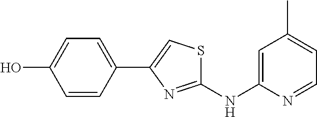 [SMILES: Cc1ccnc(Nc2nc(-c3ccc(O)cc3)cs2)c1] | >10.0 | 283.4 | 58.0 | 4.07 | 4/2 |
| IND52 | 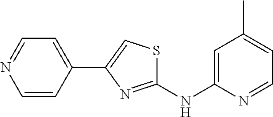 [SMILES: Cc1ccnc(Nc2nc(-c3ccncc3)cs2)c1] | 4.95 | 268.3 | 50.7 | 3.13 | 4/1 |
| IND57 | 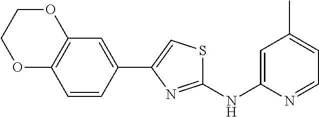 [SMILES: Cc1ccnc(Nc2nc(-c3ccc4c(c3)OCCO4)cs2)c1] | >10.0 | 325.4 | 56.3 | 3.95 | 5/1 |
| IND64 | 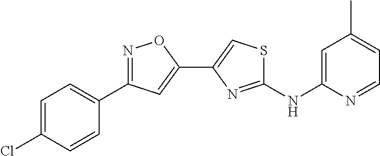 [SMILES: Cc1ccnc(Nc2nc(-c3cc(-c4ccc(Cl)cc4)no3)cs2)c1] | 0.85 | 368.8 | 64.1 | 4.91 | 5/1 |
| IND81 | 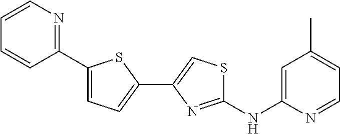 [SMILES: Cc1ccnc(Nc2nc(-c3ccc(-c4ccccn4)s3)cs2)c1] | 1.28 | 350.5 | 50.7 | 4.83 | 4/1 |

HBA = H-bond acceptor.
HBD = H-bond donor.

TABLE 9
AMT analogs synthesized and tested for potency, with calculated parameters.

| Compound | Structure | EC₅₀ (μM) | MW | tPSA | xlogP | HBA/HBD |
|---|---|---|---|---|---|---|
| IND42 | 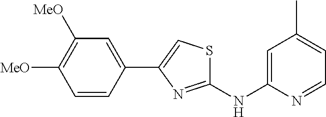 [SMILES: COc1ccc(-c2csc(Nc3cc(C)ccn3)n2)cc1OC] | 1.00 | 327.4 | 56.3 | 4.37 | 5/1 |
| IND43 | 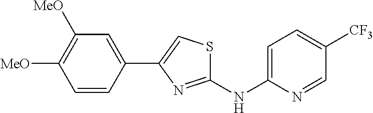 [SMILES: COc1ccc(-c2csc(Nc3ccc(C(F)(F)F)cn3)n2)cc1OC] | 2.53 | 381.4 | 56.3 | 5.08 | 5/1 |
| IND44 | 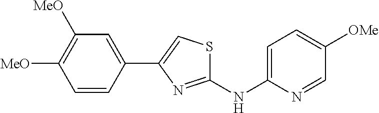 [SMILES: COc1ccc(Nc2nc(-c3ccc(OC)c(OC)c3)cs2)nc1] | 0.99 | 343.4 | 65.5 | 4.05 | 6/1 |
| IND135 | 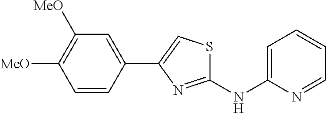 [SMILES: COc1ccc(-c2csc(Nc3ccccn3)n2)cc1OC] | 1.23 | 313.4 | 56.3 | 4.09 | 5/1 |
| IND82 | 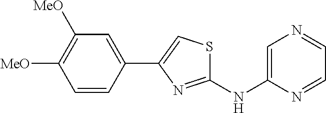 [SMILES: COc1ccc(-c2csc(Nc3cnccn3)n2)cc1OC] | 15.6 | 314.4 | 69.2 | 2.78 | 6/1 |
| IND47 | 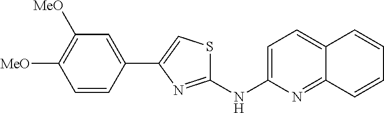 [SMILES: COc1ccc(-c2csc(Nc3ccc4ccccc4n3)n2)cc1OC] | 0.426 | 363.4 | 56.3 | 5.51 | 5/1 |
| IND46 | 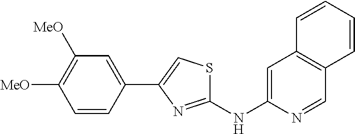 [SMILES: COc1ccc(-c2csc(Nc3cc4ccccc4cn3)n2)cc1OC] | 0.109 | 363.4 | 56.3 | 5.38 | 5/1 |
| IND85 | 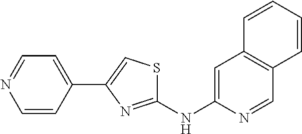 [SMILES: c1ccc2cc(Nc3nc(-c4ccncc4)cs3)ncc2c1] | 0.307 | 304.4 | 50.7 | 4.14 | 4/1 |
| IND86 | 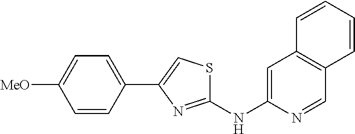 [SMILES: COc1ccc(-c2csc(Nc3cc4ccccc4cn3)n2)cc1] | 0.342 | 333.4 | 47.0 | 5.41 | 4/1 |
| IND91 | 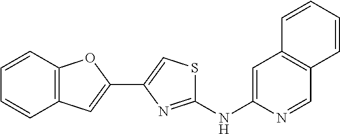 [SMILES: c1ccc2cc(Nc3nc(-c4cc5ccccc5o4)cs3)ncc2c1] | 0.923 | 343.4 | 51.3 | 6.25 | 4/1 |
| IND111 | 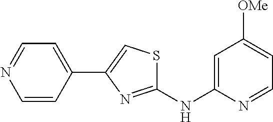 [SMILES: COc1ccnc(Nc2nc(-c3ccncc3)cs2)c1] | 0.605 | 284.3 | 59.9 | 2.82 | 5/1 |
| IND119 | 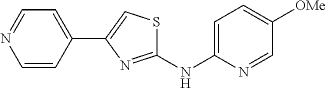 [SMILES: COc1ccc(Nc2nc(-c3ccncc3)cs2)nc1] | 1.32 | 284.2 | 59.9 | 2.82 | 5/1 |
| IND121 | 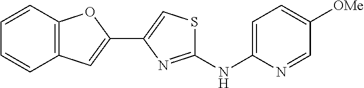 [SMILES: COc1ccc(Nc2nc(-c3cc4ccccc4o3)cs2)nc1] | 1.74 | 323.4 | 60.5 | 4.93 | 5/1 |
| IND120 | 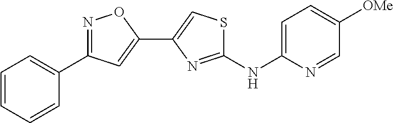 [SMILES: COc1ccc(Nc2nc(-c3cc(-c4ccccc4)no3)cs2)nc1] | 0.229 | 350.4 | 73.4 | 3.97 | 6/1 |
| IND112 | 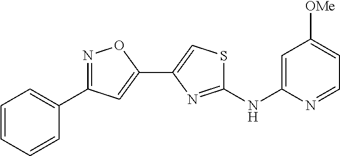 [SMILES: COc1ccnc(Nc2nc(-c3cc(-c4ccccc4)no3)cs2)c1] | 0.251 | 350.4 | 73.4 | 3.97 | 6/1 |
| IND122 | 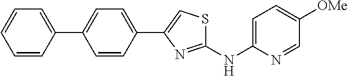 [SMILES: COc1ccc(Nc2nc(-c3ccc(-c4ccccc4)cc3)cs2)nc1] | 0.432 | 359.4 | 47.0 | 6.04 | 4/1 |

HBA = H-bond acceptor.
HBD = H-bond donor.

TABLE 10
Solubility and permeability in MDCK-MDR1 cells of 10 selected aminothiazole compounds.

| | Solubility (μM) | | | | Permeability in MDCK-MDR1 Cells | | |
|---|---|---|---|---|---|---|---|
| | under Various Conditions | | | | Pₐₚₚ (A-B) × | Pₐₚₚ (A-B) × | |
| Compound | pH 2.0 | pH 7.4 | FaSSIF | Media | 10⁻⁶ cm/sec | 10⁻⁶ cm/sec | Efflux Ratio |
| IND22 | 12 | <1 | 59 | 20 | 9.5 | 8.4 | 0.9 |
| IND24 | 1 | <1 | 118 | >250 | 5.6 | 7.3 | 1.3 |
| IND33 | 1 | <1 | 70 | 225 | 6.8 | 8.3 | 1.2 |
| IND44 | 1 | <1 | <1 | 3 | 20.1 | 24.4 | 1.2 |
| IND46 | <1 | <1 | 5 | 174 | 13.2 | 10.5 | 0.8 |
| IND47 | <1 | <1 | 5 | 52 | 9.2 | 8.9 | 1 |
| IND52 | 215 | 1 | 2 | 76 | 36.2 | 48.7 | 1.3 |
| IND81 | 78 | <1 | 3 | 207 | 16 | 20.9 | 1.3 |
| IND85 | 196 | <1 | 3 | 116 | 3.03 | 4.44 | 1.5 |
| IND91 | na | na | na | 151 | na | na | na |

FaSSIF = Fasted state simulated intestinal fluid.
Media = Media used in the cell-based assays.
na = not analyzed.

TABLE 11
Metabolic stability of 10 selected aminothiazole drug candidates.

| | t_{1/2} (min) | | | Hepatic Extraction Ratio | | |
|---|---|---|---|---|---|---|
| Compound | Mouse | Rat | Human | Mouse | Rat | Human |
| IND22 | 49 | 106* | >60** | 0.7 | 0.4 | nd |
| IND24 | >60* | >60* | >60* | nd | nd | nd |
| IND33 | 99* | 151* | 54 | 0.5 | 0.3 | 0.7 |
| IND44 | 29 | 45 | 58 | 0.8 | 0.6 | 0.7 |
| IND46 | 75* | 83* | >60** | 0.6 | 0.4 | nd |
| IND47 | 72* | 69* | >60** | 0.6 | 0.5 | nd |
| IND52 | 6 | 16 | 36 | 0.95 | 0.8 | 0.8 |
| IND81 | 19 | 58 | 52 | 0.8 | 0.5 | 0.7 |
| IND85 | 15 | 18 | 53 | 0.9 | 0.8 | 0.7 |
| IND91 | na | na | na | na | na | na |

na = not analyzed;
nd = not determinable,
*= extrapolated.
**= little or no slope for extra polation.

TABLE 12
Metabolic stability and hepatic extraction ratio in animal (by gender) and human microsomes.

| | t_{1/2} (min) | | | | | | | Hepatic Extraction Ratio | | | | | | |
|---|---|---|---|---|---|---|---|---|---|---|---|---|---|---|
| | Dog | | Rat | | Mouse | | | Dog | | Rat | | Mouse | | |
| Compound | Female | Male | Female | Male | Female | Male | Human | Female | Male | Female | Male | Female | Male | Human |
| IND24 | 51 | 140* | 770* | 430* | 290* | 190* | 1400* | 0.56 | 0.32 | 0.04 | 0.08 | 0.17 | 0.24 | 0.05 |
| IND81 | 26 | 23 | 290* | 70 | 26 | 23 | 40 | 0.71 | 0.74 | 0.11 | 0.34 | 0.70 | 0.73 | 0.63 |
| IND54304 | 16 | 20 | 96 | 96 | 34 | 25 | 130* | 0.80 | 0.79 | 0.27 | 0.27 | 0.64 | 0.71 | 0.34 |

*Extrapolated.

TABLE 13
Percentage binding of IND24 and IND81 in mouse brain homogenate
and mouse, rat, dog, and human plasma at 1 and 10 μM. Mean
percentage ± SD (N = 3 except for warfarin where N = 2).

| Compound | Species | 1 μM | 10 μM |
|---|---|---|---|
| IND24 | Dog | 95.1 ± 1.38 | 95.0 ± 0.32 |
| | Rat | 93.4 ± 2.13 | 94.0 ± 1.04 |
| | Mouse | 93.4 ± 1.55 | 95.8 ± 0.14 |
| | Mouse (Brain) | 91.8 ± 0.35 | 93.1 ± 1.69 |
| | Human | 95.1 ± 1.03 | 95.4 ± 1.17 |
| IND81 | Dog | 94.4 ± 1.88 | 94.7 ± 1.03 |
| | Rat | 94.2 ± 0.48 | 95.5 ± 0.66 |
| | Mouse | 93.5 ± 1.10 | 96.0 ± 0.04 |
| | Mouse (Brain) | 90.6 ± 1.49 | 90.7 ± 1.43 |
| | Human | 94.2 ± 3.21 | 95.2 ± 0.86 |
| Warfarin | Dog | 96.2 ± 0.0017 | 97.2 ± 0.0002 |
| | Rat | 99.4 ± 0.0001 | 99.0 ± 0.0027 |
| | Mouse | 89.2 ± 0.0044 | 89.5 ± 0.0060 |
| | Human | 99.3 ± 0.0023 | 99.4 ± 0.0004 |

TABLE 14
Observed and calculated molecular ions and mass spectral fragment ions of IND24 and its metabolites (top panel),
and IND81 and its metabolites (bottom panel) following incubation with human, mouse, rat and dog liver microsomes.

| | | | Molecular | | | | |
|---|---|---|---|---|---|---|---|
| Metab- | | | Ions | Fragment Ions | | | |
| olite | Structure | | MH⁺ | a | b | c | d |
| Parent IND24 | 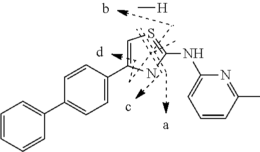 | Ob- served Calcu- lated | 344.1212 344.1216 | 226.0687 226.0684 | 209.0421 209.0420 | 193.0887 193.0886 | 165.0698 165.0687 |
| M1 | 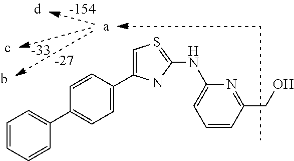 | Ob- served Calcu- lated | 360.1162 360.1165 | 342.1060 342.1059 | 315.0951 315.0950 | 309.1262 309.1260 | 161.0167 161.0167 |
| M2/ M3/ M4 | 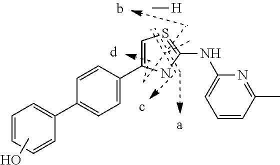 | Ob- served Calcu- lated | 360.1158 360.1165 | 242.0634 242.0634 | 225.0369 225.0363 | 209.0835 209.0835 | 181.0647 181.0647 |
| M5 | 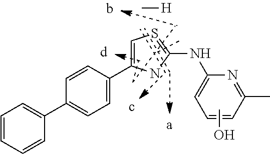 | Ob- served Calcu- lated | 360.1160 360.1165 | 226.0686 226.0684 | 209.0421 209.0420 | 193.0886 193.0886 | 165.0698 165.0687 |

| | | | Molecular | | | | | |
|---|---|---|---|---|---|---|---|---|
| Metab- | | | Ions | Fragment Ions | | | | |
| olite | Structure | | MH⁺ | a | b | c | d | e |
| Parent IND81 | 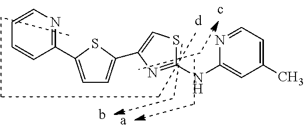 [SMILES: Cc1ccnc(Nc2nc(-c3ccc(-c4ccccn4)s3)cs2)c1] | Ob- served Calcu- lated | 351.0729 351.0732 | 243.0048 243.0039 | 233.0205 233.0202 | 218.0096 218.0092 | 206.0095 206.0092 | |
| M1/M3 | 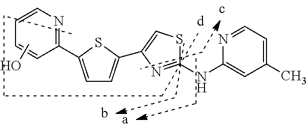 | Ob- served Calcu- lated | 367.0680 367.0681 | 258.9997 258.9988 | 249.0154 249.0150 | 234.0045 234.0042 | 222.0045 222.0041 | |
| M2 | 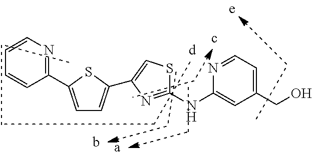 [SMILES: OCc1ccnc(Nc2nc(-c3ccc(-c4ccccn4)s3)cs2)c1] | Ob- served Calcu- lated | 367.0679 351.0732 | 243.0048 243.0039 | 233.0204 233.0202 | 218.0095 218.0092 | 206.0094 206.0092 | 349.0576 349.0579 |

TABLE 15
CYP phenotyping of IND24 and IND81 in 1 μM human liver microsomes.

| | | IND24 | | | | IND81 | | | |
|---|---|---|---|---|---|---|---|---|---|
| | | Measured | | | | Measured | | | CYP |
| | | Conc. | % | Δ % | CYP | Conc. | % | Δ % | In- |
| Enzyme | Treatment | (uM) | Disappearance | Disappearrance | Involvement | (uM) | Disappearance | Disappearance | volvement |

Irreversible Incubation Condition

| | | | | | | | | | |
|---|---|---|---|---|---|---|---|---|---|
| Negative | No NADPH/No | 0.622 | | | | 0.899 | 0 | | |
| Control | Inhibitor | | | | | | | | |
| Maximum | NADPH/No | 0.733 | 0 (−18) | 0 | | 0.143 | 84 | 0 | |
| Metabolism | Inhibitor | | | | | | | | |
| CYP1A2 | NADPH + | 0.766 | 0 (−23) | 0 | Unlikely | 0.534 | 41 | 43 | Unlikely |
| | Furafylline | | | | | | | | |

Reversible Incubation Condition

| | | | | | | | | | |
|---|---|---|---|---|---|---|---|---|---|
| Negative | No NADPH/No | 0.654 | 0 | | | 0.664 | 0 | | |
| Control | Inhibitor | | | | | | | | |
| Maximum | NADPH/No | 0.655 | 0 | 0 | | 0.226 | 66 | 0 | |
| Metabolism | Inhibitor | | | | | | | | |
| CYP2C9 | NADPH + | 0.566 | 14 | 0 (−14) | Unlikely | 0.238 | 64 | 1.8 | Unlikely |
| | Sulphaphenazol | | | | | | | | |
| CYP2C19 | NADPH + N-3-B | 0.537 | 18 | 0 (−18) | Unlikely | 0.283 | 57 | 8.5 | Possible |
| CYP2D6 | NADPH + | 0.555 | 15 | 0 (−15) | Unlikely | 0.300 | 55 | 11 | Likely |
| | Quinidine | | | | | | | | |
| CYP3A4 | NADPH + | 0.557 | 15 | 0 (−15) | Unlikely | 0.324 | 51 | 15 | Likely |
| | Ketoconazole | | | | | | | | |

For Δ % Disappearance, the involvement of the CYP considered unlikely for values <5; possible for values 5-10; and likely for values >10.

TABLE 16
Single-dose plasma pharmacokinetics of IND24, IND81 and IND54304 in female FVB
mice. Intravenous (IV) dose was 1 mg/kg; dose by oral gavage (PO) was 40 mg/kg.
Mean ± SD shown for Cₘₐₓ and AUC values (N = 2).

| | | | Cₘₐₓ | | AUCₗₐₛₜ | | |
|---|---|---|---|---|---|---|---|
| | Route/ | t_{1/2} | [(ng/mL) or | tₘₐₓ | [(ng/mL * h) or | | Brain/Plasma |
| Compound | Matrix | (h) | (ng/g)] | (h) | (ng/g * h)] | % F | AUCₗₐₛₜ Ratio |
| IND24 | IV/Plasma | 2.15 | 856 ± 908 | 0.083 | 953 ± 374 | | |
| | PO/Plasma | 4.65 | 569 ± 38.1 | 2 | 3830 ± 450 | 40.2 | |
| | PO/Brain | nd | 840 ± 252 | 4 | 9998 ± 417 | | 2.61 |
| IND81 | IV/Plasma | 1.17 | 183 ± 96.6 | 0.083 | 106 ± 12.4 | | |
| | PO/Plasma | 0.97 | 152 ± 10.6 | 0.5 | 288 ± 136 | 27.2 | |
| | PO/Brain | nd | 568 ± 186 | 0.5 | 1592 ± 794 | | 5.53 |
| IND54304 | IV/Plasma | 0.21 | 466 ± 37.4 | 0.083 | 219 ± 33.0 | | |
| | PO/Plasma | 1.00 | 220 ± 99.4 | 0.5 | 544 ± 16.8 | 24.8 | |
| | PO/Brain | nd | 123 ± 42.7 | 2 | 282 ± 27.2 | | 0.52 |

Cₘₐₓ values in ng/mL for plasma and ng/g for brain.
AUCₗₐₛₜ values in ng/mL * h for plasma and ng/g * h for brain.

TABLE 17
“Steady-state” concentrations (Cₛₛ) of 10 2-AMT drug candidates in brain and plasma
following three-day dosing in liquid diet. Mean ± SD values shown (N = 2).

| | Brain Concentration (μM) | | | | Plasma Concentration (μM) | | | |
|---|---|---|---|---|---|---|---|---|
| Compound | 40 mg/kg | 80 mg/kg | 130 mg/kg | 210 mg/kg | 40 mg/kg | 80 mg/kg | 130 mg/kg | 210 mg/kg |
| IND22 | 1.31 ± 0.39 | 2.70 ± 0.52 | 3.74 ± 0.41 | 3.23 ± 2.62 | 1.49 ± 0.15 | 2.14 ± 0.58 | 2.50 ± 0.67 | 1.98 ± 1.24 |
| IND24 | 8.70 ± 1.46 | 19.3 ± 2.28 | 31.8 ± 5.46 | 37.4 ± 9.06 | 8.19 ± 8.09 | 5.55 ± 1.18 | 11.7 ± 4.85 | 18.9 ± 20.5 |
| IND81 | 3.00 ± 0.52 | 7.45 ± 1.00 | 13.0 ± 2.81 | 19.3 ± 3.24 | 4.34 ± 1.19 | 9.15 ± 0.67 | 15.1 ± 5.20 | 17.9 ± 2.45 |
| IND52 | n.d. | 0.76 ± 1.09 | 2.34 ± 2.13 | 7.65 ± 5.57 | 0.01 ± 0.01 | 0.67 ± 1.00 | 1.93 ± 1.58 | 10.9 ± 8.44 |
| IND33 | 0.88 ± 0.68 | 5.39 ± 2.93 | 23.3 ± 13.2 | 12.6 ± 8.62 | 0.65 ± 0.38 | 2.85 ± 1.30 | 4.74 ± 1.31 | 14.2 ± 2.09 |
| IND91 | n.d. | 0.19 ± 0.09 | 0.26 ± 0.12 | 0.28 ± 0.04 | 0.26 ± 0.13 | 1.55 ± 0.36 | 1.74 ± 0.31 | 3.06 ± 0.56 |
| IND85 | 0.02 ± 0.03 | 0.08 ± 0.03 | 0.04 ± 0.03 | 0.10 ± 0.07 | n.d. | n.d. | n.d. | n.d. |
| IND44 | n.d. | n.d. | n.d. | n.d. | 0.06 ± 0.01 | 0.09 ± 0.04 | 0.13 ± 0.08 | 0.18 ± 0.09 |
| IND46 | 0.03 ± 0.03 | 0.06 ± 0.07 | 0.14 ± 0.04 | 0.32 ± 0.11 | 0.05 ± 0.02 | 0.06 ± 0.03 | 0.08 ± 0.04 | 0.14 ± 0.03 |
| IND47 | 0.40 ± 0.28 | n.d. | 0.40 ± 0.51 | 0.23 ± 0.26 | 0.13 ± 0.10 | 0.03 ± 0.02 | 0.06 ± 0.02 | 0.12 ± 0.05 |

n.d., not determined because concentrations were below the lower level for quantification.

TABLE 18
AMT compounds

| | | | | | | Analiza | |
|---|---|---|---|---|---|---|---|
| | | | | | | solubility | |
| | | | | | | (uM) | |
| | | | | | | pH | pH |
| | IND# | Structure | Compound | Batch | Quantity_(mg) | 1.2 | 7.4 |
| 1 | IND- 0000024 | 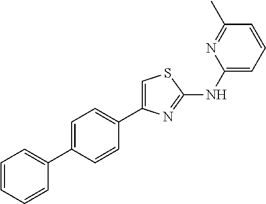 [SMILES: Cc1cccc(Nc2nc(-c3ccc(-c4ccccc4)cc3)cs2)n1] | | 052410 | enough | <0.7 | 3.2 |
| 2 | IND- 0000081 | 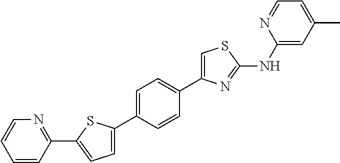 [SMILES: Cc1ccnc(Nc2nc(-c3ccc(-c4ccc(-c5ccccn5)s4)cc3)cs2)c1] | | | enough | | |
| 3 | IND- 0116138 | 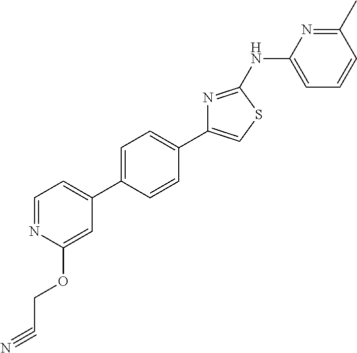 [SMILES: Cc1cccc(Nc2nc(-c3ccc(-c4ccnc(OCC#N)c4)cc3)cs2)n1] | IND-0116138 (AMT082_3O_10) | E207- 3548-006 | 136 | >150 | <0.4 |
| 4 | IND- 0116096 | 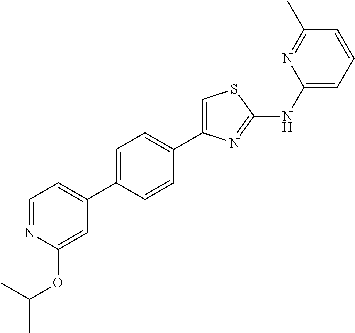 [SMILES: Cc1cccc(Nc2nc(-c3ccc(-c4ccnc(OC(C)C)c4)cc3)cs2)n1] | AMT082_3O_9 | E823- 3083-009 | 57 | >150 | 3.1 |
| 5 | IND- 0116103 | 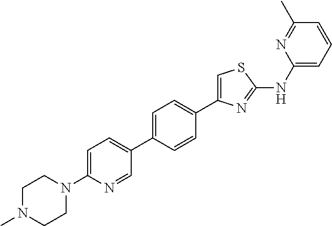 [SMILES: Cc1cccc(Nc2nc(-c3ccc(-c4ccc(N5CCN(C)CC5)nc4)cc3)cs2)n1] | AMT082_4N_3 | E207- 3095-019 | 128 | 37.1 | 4.4 |
| 6 | IND- 0116099 | 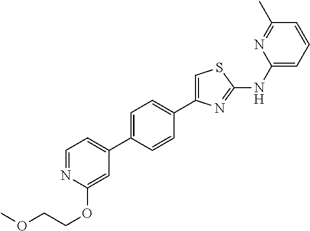 [SMILES: COCCOc1cc(-c2ccc(-c3csc(Nc4cccc(C)n4)n3)cc2)ccn1] | AMT082_3O_7 | E823- 3083-010 | 74 | 125.5 | <0.5 |
| 7 | IND- 0116134 | 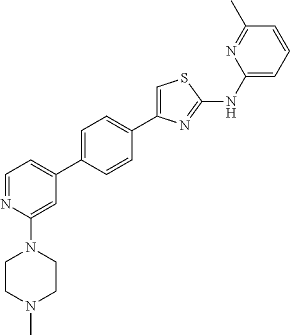 [SMILES: Cc1cccc(Nc2nc(-c3ccc(-c4ccnc(N5CCN(C)CC5)c4)cc3)cs2)n1] | AMT082_3N_8 | E207- 3095-022 | 110 | 81.8 | 40.2 |
| 8 | IND- 0116094 | 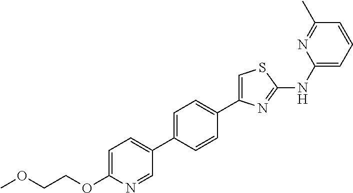 [SMILES: COCCOc1ccc(-c2ccc(-c3csc(Nc4cccc(C)n4)n3)cc2)cn1] | IND-0116094 (AMT082_4O_3) | E823- 3083-050 | 206 | 62.8 | 2.2 |
| 9 | IND- 0116044 | 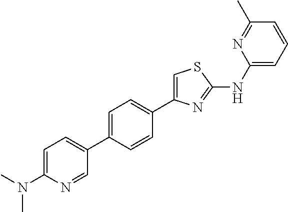 [SMILES: Cc1cccc(Nc2nc(-c3ccc(-c4ccc(N(C)C)nc4)cc3)cs2)n1] | AMT082_4N_1 | E207- 3095-015 | 127 | 77.1 | 14.3 |
| 10 | IND- 0116095 | 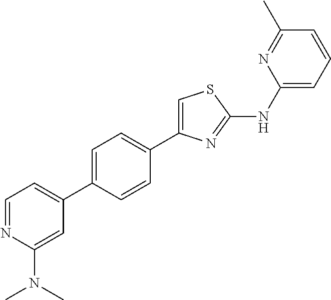 [SMILES: Cc1cccc(Nc2nc(-c3ccc(-c4ccnc(N(C)C)c4)cc3)cs2)n1] | AMT082_3N_6 | E207- 3095-010 | 33 | >150 | 1.6 |
| 11 | IND- 0116097 | 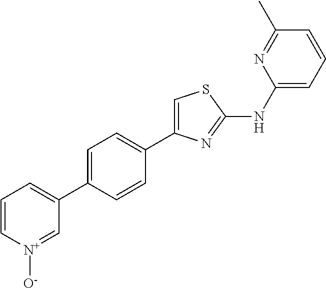 [SMILES: Cc1cccc(Nc2nc(-c3ccc(-c4ccc[n+]([O-])c4)cc3)cs2)n1] | IND-0116097 (AMT082_3Noxide_15) | E823- 3083-049 | 188 | 49 | 5.1 |
| 12 | IND- 0116165 | 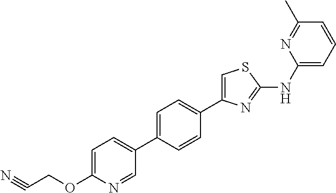 [SMILES: Cc1cccc(Nc2nc(-c3ccc(-c4ccc(OCC#N)nc4)cc3)cs2)n1] | AMT082_4O_5 | E207- 3095-028 | 23 | 2.2 | <0.4 |

TABLE 19
AMT compounds

| | | | | | | Analiza | |
|---|---|---|---|---|---|---|---|
| | | | | | | solubility | |
| | | | | | | (uM) | |
| | | | | | | pH | pH |
| | IND# | Structure | Compound | Batch | Quantity_(mg) | 1.2 | 7.4 |
| 1 | IND- 0000024 | 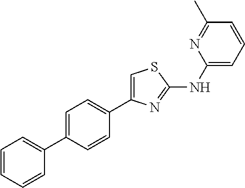 [SMILES: Cc1cccc(Nc2nc(-c3ccc(-c4ccccc4)cc3)cs2)n1] | | 052410 | enough | <0.7 | 3.2 |
| 2 | IND- 0030410 | 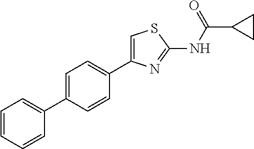 [SMILES: O=C(Nc1nc(-c2ccc(-c3ccccc3)cc2)cs1)C1CC1] | | 012711 | enough | 17.7 | 12.7 |
| 3a | IND- 0116138 | 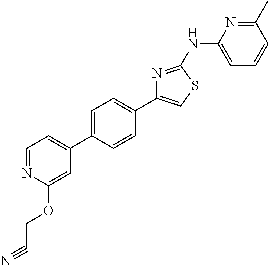 [SMILES: Cc1cccc(Nc2nc(-c3ccc(-c4ccnc(OCC#N)c4)cc3)cs2)n1] | IND-0116138 (AMT082_3O_10) | E207- 3548- 006 | 136 | >150 | <0.4 |
| 3b | IND- 0116138 | 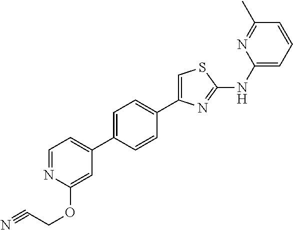 [SMILES: Cc1cccc(Nc2nc(-c3ccc(-c4ccnc(OCC#N)c4)cc3)cs2)n1] | AMT082_3O_10 | E207- 3095- 021 | 87 | | |
| 4 | IND- 0116096 | 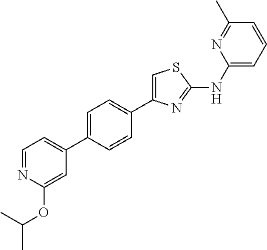 [SMILES: Cc1cccc(Nc2nc(-c3ccc(-c4ccnc(OC(C)C)c4)cc3)cs2)n1] | AMT082_3O_9 | E823- 3083- 009 | 57 | >150 | 3.1 |
| 5 | IND- 0116102 | 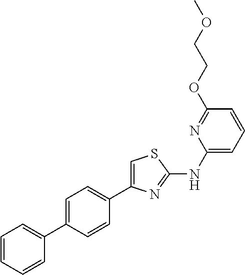 [SMILES: COCCOc1cccc(Nc2nc(-c3ccc(-c4ccccc4)cc3)cs2)n1] | AMT082_deMe_12 | E765- 3082- 030 | 49 | 2.1 | 2.7 |
| 6 | IND- 0116103 | 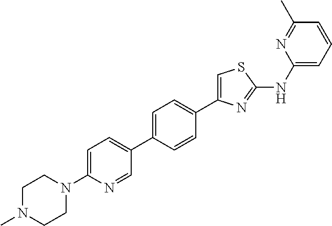 [SMILES: Cc1cccc(Nc2nc(-c3ccc(-c4ccc(N5CCN(C)CC5)nc4)cc3)cs2)n1] | AMT082_4N_3 | E207- 3095- 019 | 128 | 37.1 | 4.4 |
| 7 | IND- 0126462 | 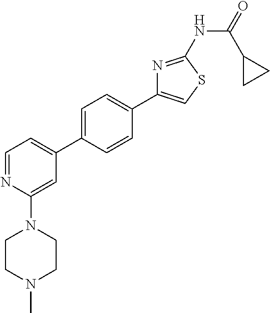 [SMILES: CN1CCN(c2cc(-c3ccc(-c4csc(NC(=O)C5CC5)n4)cc3)ccn2)CC1] | AMT410_3N-3 | E882- 3459- 014 | 27 | | |
| 8 | IND- 0126480 | 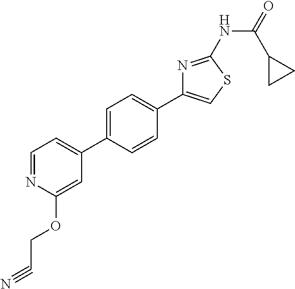 [SMILES: N#CCOc1cc(-c2ccc(-c3csc(NC(=O)C4CC4)n3)cc2)ccn1] | AMT410_3O-1 | E207- 3548- 005 | 42 | | |
| 9 | IND- 0116099 | 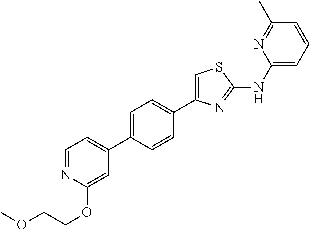 [SMILES: COCCOc1cc(-c2ccc(-c3csc(Nc4cccc(C)n4)n3)cc2)ccn1] | AMT082_3O_7 | E823- 3083- 010 | 74 | 125.5 | <0.5 |
| 10 | IND- 0116134 | 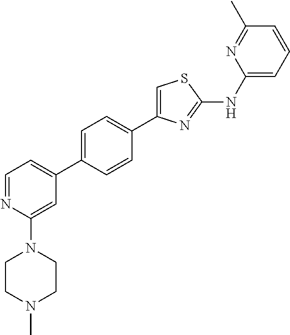 [SMILES: Cc1cccc(Nc2nc(-c3ccc(-c4ccnc(N5CCN(C)CC5)c4)cc3)cs2)n1] | AMT082_3N_8 | E207- 3095- 022 | 110 | 81.8 | 40.2 |
| 11a | IND- 0116094 | 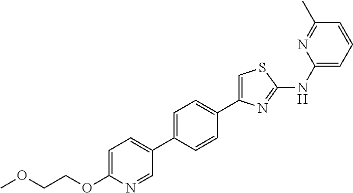 [SMILES: COCCOc1ccc(-c2ccc(-c3csc(Nc4cccc(C)n4)n3)cc2)cn1] | IND-0116094 (AMT082_4O_3) | E823- 3083- 050 | 206 | 62.8 | 2.2 |
| 11b | IND- 0116094 | 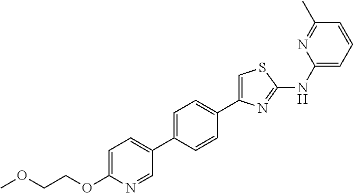 [SMILES: COCCOc1ccc(-c2ccc(-c3csc(Nc4cccc(C)n4)n3)cc2)cn1] | AMT082_4O_2 | E207- 3095- 005 | 123 | | |
| 12 | IND- 0116044 | 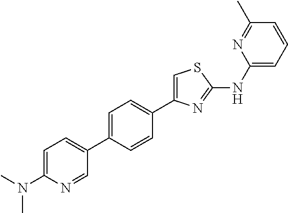 [SMILES: Cc1cccc(Nc2nc(-c3ccc(-c4ccc(N(C)C)nc4)cc3)cs2)n1] | AMT082_4N_1 | E207- 3095- 015 | 127 | 77.1 | 14.3 |
| 13 | IND- 116123 | 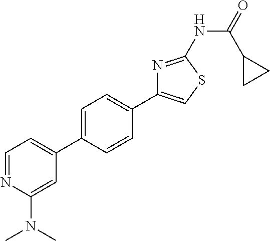 [SMILES: CN(C)c1cc(-c2ccc(-c3csc(NC(=O)C4CC4)n3)cc2)ccn1] | AMT410_3N-4 | E207- 3548- 002 | 26 | >150 | <0.5 |
| 14 | IND- 0116095 | 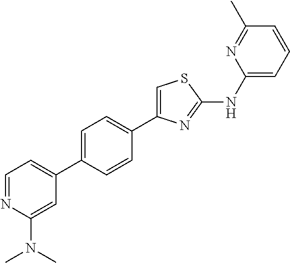 [SMILES: Cc1cccc(Nc2nc(-c3ccc(-c4ccnc(N(C)C)c4)cc3)cs2)n1] | AMT082_3N_6 | E207- 3095- 010 | 33 | >150 | 1.6 |
| 15 | IND- 0126437 | 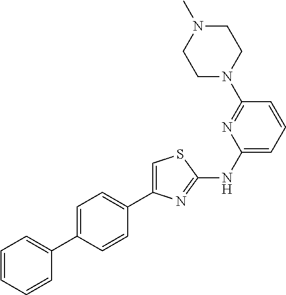 [SMILES: CN1CCN(c2cccc(Nc3nc(-c4ccc(-c5ccccc5)cc4)cs3)n2)CC1] | AMT082_deMe_1 | E1000- 3264- 033 | 39 | | |
| 16 | IND- 0126446 | 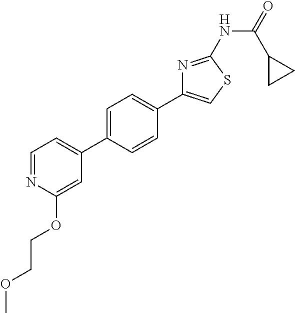 [SMILES: COCCOc1cc(-c2ccc(-c3csc(NC(=O)C4CC4)n3)cc2)ccn1] | AMT410_3O-2 | E882- 3459- 008 | 106 | | |
| 17a | IND- 0116097 | 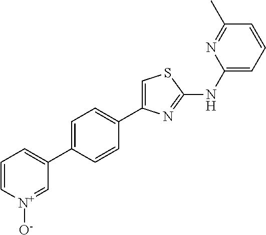 [SMILES: Cc1cccc(Nc2nc(-c3ccc(-c4ccc[n+]([O-])c4)cc3)cs2)n1] | IND-0116097 (AMT082_3Noxide_15) | E823- 3083- 049 | 188 | 49 | 5.1 |
| 17b | IND- 0116097 | 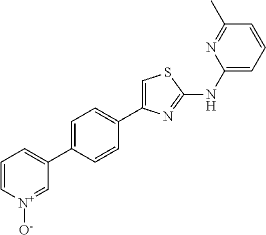 [SMILES: Cc1cccc(Nc2nc(-c3ccc(-c4ccc[n+]([O-])c4)cc3)cs2)n1] | AMT082_3Noxide_15 | E207- 3095- 009 | 42 | | |
| 18 | IND- 0116165 | 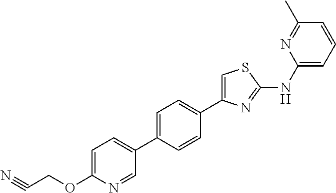 [SMILES: Cc1cccc(Nc2nc(-c3ccc(-c4ccc(OCC#N)nc4)cc3)cs2)n1] | AMT082_4O_5 | E207- 3095- 028 | 23 | 2.2 | <0.4 |
| 19 | IND- 0126461 | 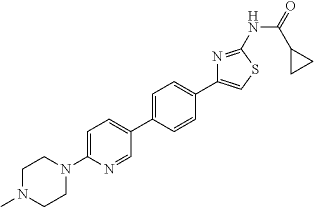 [SMILES: CN1CCN(c2ccc(-c3ccc(-c4csc(NC(=O)C5CC5)n4)cc3)cn2)CC1] | AMT410_4N-3 | E882- 3459- 013 | 17 | | |
| 20 | IND- 0116091 | 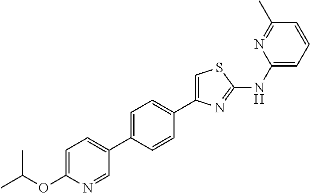 [SMILES: Cc1cccc(Nc2nc(-c3ccc(-c4ccc(OC(C)C)nc4)cc3)cs2)n1] | AMT082_4O_4 | E207- 3095- 001 | 42 | 13.1 | 3.2 |
| 21 | 1ND- 0126443 | 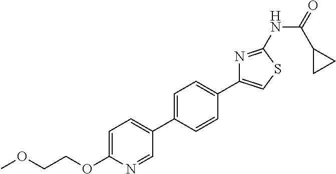 [SMILES: COCCOc1ccc(-c2ccc(-c3csc(NC(=O)C4CC4)n3)cc2)cn1] | AMT410_4O-2 | E882- 3459- 006 | 28 | | |
| 22 | IND- 0116092 | 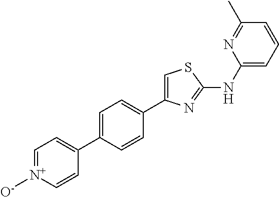 [SMILES: Cc1cccc(Nc2nc(-c3ccc(-c4cc[n+]([O-])cc4)cc3)cs2)n1] | AMT082_4Noxide_14 | E207- 3095- 016 | 100 | 17.6 | 15.2 |
| 23 | IND- 0126464 | 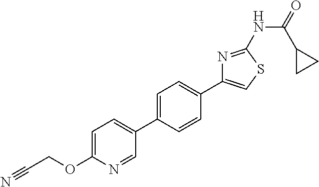 [SMILES: N#CCOc1ccc(-c2ccc(-c3csc(NC(=O)C4CC4)n3)cc2)cn1] | AMT410_4O-1 | E207- 3548- 004 | 26 | | |
| 24 | IND- 0116142 | 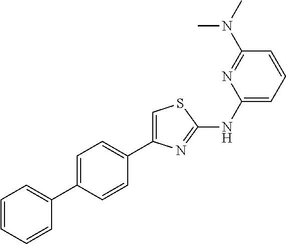 [SMILES: CN(C)c1cccc(Nc2nc(-c3ccc(-c4ccccc4)cc3)cs2)n1] | AMT082_deMe_11 | E765- 3082- 036 | 54 | 16.1 | <0.5 |
| 25 | IND- 0126465 | 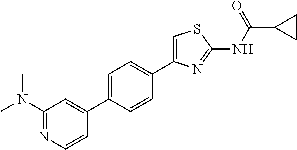 [SMILES: CN(C)c1cc(-c2ccc(-c3csc(NC(=O)C4CC4)n3)cc2)ccn1] | | | | | |
| 26 | IND- 0114338 | 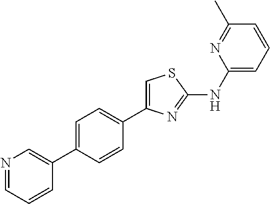 [SMILES: Cc1cccc(Nc2nc(-c3ccc(-c4cccnc4)cc3)cs2)n1] | SMDC | | | | |
| 27 | IND- 0114337 | 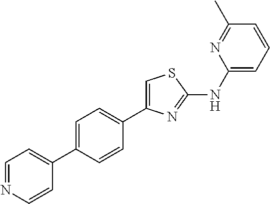 [SMILES: Cc1cccc(Nc2nc(-c3ccc(-c4ccncc4)cc3)cs2)n1] | SMDC | | | | |

TABLE 20
AMT compounds

| | | | | Analiza | Analiza |
|---|---|---|---|---|---|
| | | | | solubility | solubility |
| | | | | (μM) pH | (μM) pH |
| Compound structure | name | MW | SMILES | 1.2 | 7.4 |
| 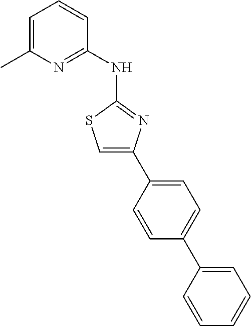 [SMILES: Cc1cccc(Nc2nc(-c3ccc(-c4ccccc4)cc3)cs2)n1] | IND- 0000024 | 343.44692 | CC4═CC═CC(═N4) NC3═NC(═CS3)C2═CC═C (C═C2)C1═CC═CC═C1 | <0.7 | 3.2 |
| 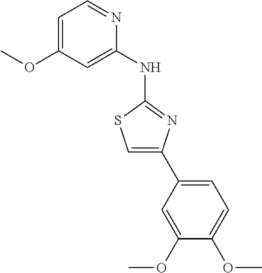 | IND- 0000115 | 343.40232 | COC3═CC═NC(═C3) NC2═NC(═CS2)C1═CC═C (OC)C(═C1)OC | | |
| 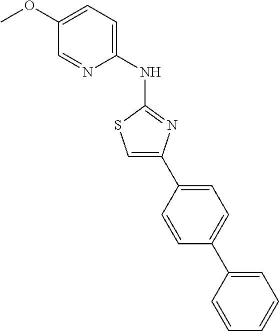 [SMILES: COc1ccc(Nc2nc(-c3ccc(-c4ccccc4)cc3)cs2)nc1] | IND- 0000122 | 359.44632 | COC4═CC═C (NC3═NC(═CS3) C2═CC═C (C═C2) C1═CC═CC═C1) N═C4 | | |
| 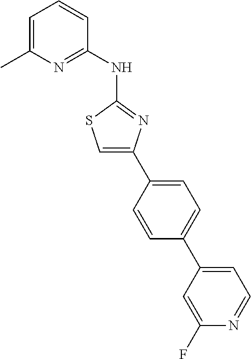 [SMILES: Cc1cccc(Nc2nc(-c3ccc(-c4ccnc(F)c4)cc3)cs2)n1] | IND- 0114335 | 362.42536 | CC4═CC═CC(═N4) NC3═NC (═CS3)C2═CC═C (C═C2) C1═CC═NC(═C1)F | | |
| 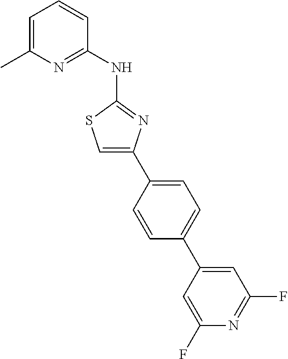 [SMILES: Cc1cccc(Nc2nc(-c3ccc(-c4cc(F)nc(F)c4)cc3)cs2)n1] | IND- 0114336 | 380.41576 | CC4═CC═CC(═N4) NC3═NC (═CS3)C2═CC═C (C═C2) C1═CC (═NC(═C1)F)F | | |
| 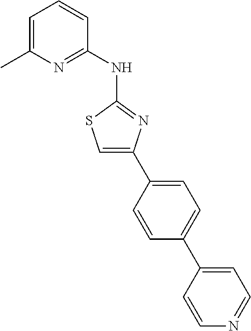 [SMILES: Cc1cccc(Nc2nc(-c3ccc(-c4ccncc4)cc3)cs2)n1] | IND- 0114337 | 344.43496 | CC4═CC═CC(═N4) NC3═NC (═CS3)C2═CC═C (C═C2) C1═CC═NC═C1 | | |
| 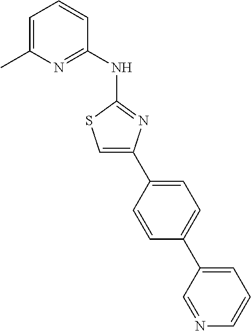 [SMILES: Cc1cccc(Nc2nc(-c3ccc(-c4cccnc4)cc3)cs2)n1] | IND- 0114338 | 344.43496 | CC4═CC═CC(═N4) NC3═NC (═CS3)C2═CC═C (C═C2) C1═CC═CN═C1 | | |
| 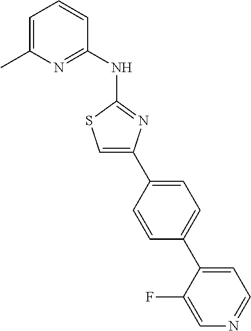 [SMILES: Cc1cccc(Nc2nc(-c3ccc(-c4ccncc4F)cc3)cs2)n1] | IND- 0114339 | 362.42536 | CC4═CC═CC(═N4) NC3═NC (═CS3)C2═CC═C (C═C2) C1═CC═NC═C1F | | |
| 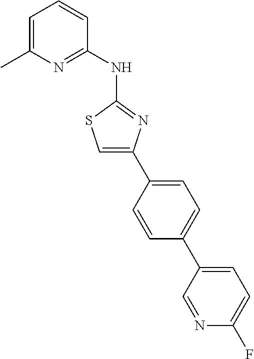 [SMILES: Cc1cccc(Nc2nc(-c3ccc(-c4ccc(F)nc4)cc3)cs2)n1] | IND- 0114340 | 362.42536 | CC4═CC═CC(═N4) NC3═NC (═CS3)C2═CC═C (C═C2) C1═CC═C(F)N═C1 | | |
| 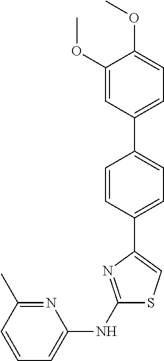 [SMILES: COc1ccc(-c2ccc(-c3csc(Nc4cccc(C)n4)n3)cc2)cc1OC] | IND- 0114341 | 403.49912 | COC4═CC═C(C═C4OC) C3═CC═C (C═C3)C2═CSC (═N2)NC1═CC═CC (═N1)C | | |
| 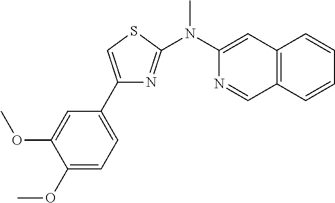 [SMILES: COc1ccc(-c2csc(N(C)c3cc4ccccc4cn3)n2)cc1OC] | IND- 0114342 | 391.48842 | CCN(C2═NC(═CS2) C1═CC═C(OC) C(═C1)OC) C4═CC3═C (C═CC═C3)C═N4 | | |
| 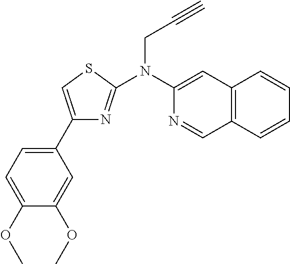 [SMILES: C#CCN(c1cc2ccccc2cn1)c1nc(-c2ccc(OC)c(OC)c2)cs1] | IND- 0114344 | 401.48312 | COC4═CC═C(C═C4OC) C3═CSC(═N3) N(CC#C) C2═CC1═C (C═CC═C1)C═N2 | | |
| 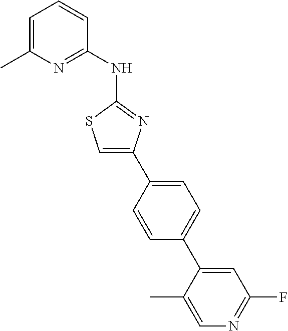 [SMILES: Cc1cccc(Nc2nc(-c3ccc(-c4cc(F)ncc4C)cc3)cs2)n1] | IND- 0116039 | 376.45206 | CC4═CC═CC (═N4)NC3═NC(═CS3) C2═CC═C(C═C2) C1═CC(═NC═C1C)F | | |
| 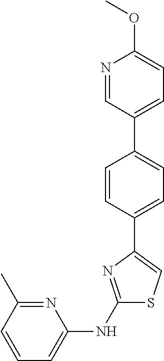 [SMILES: COc1ccc(-c2ccc(-c3csc(Nc4cccc(C)n4)n3)cc2)cn1] | IND- 0116040 | 374.46106 | COC4═CC═C (C═N4)C3═CC═C (C═C3)C2═CSC (═N2)NC1═CC═CC (═N1)C | | |
| 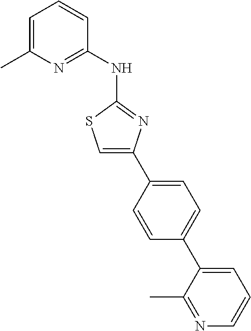 [SMILES: Cc1cccc(Nc2nc(-c3ccc(-c4cccnc4C)cc3)cs2)n1] | IND- 0116041 | 358.46166 | CC4═CC═CC (═N4)NC3═NC (═CS3)C2═CC═C (C═C2) C1═CC═CN═C1C | | |
| 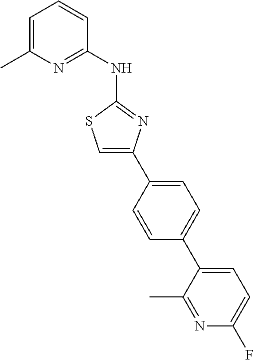 [SMILES: Cc1cccc(Nc2nc(-c3ccc(-c4ccc(F)nc4C)cc3)cs2)n1] | IND- 0116042 | 376.45206 | CC4═CC═CC (═N4)NC3═NC (═CS3)C2═CC═C (C═C2) C1═CC═C(F)N═C1C | | |
| 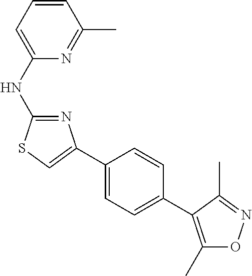 [SMILES: Cc1cccc(Nc2nc(-c3ccc(-c4c(C)noc4C)cc3)cs2)n1] | IND- 0116043 | 362.45036 | CC4═NOC (═C4C3═CC═C (C═C3)C2═CSC (═N2)NC1═CC═CC (═N1)C)C | | |
| 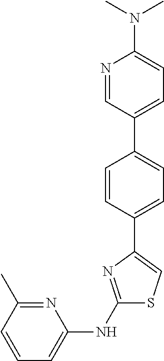 [SMILES: Cc1cccc(Nc2nc(-c3ccc(-c4ccc(N(C)C)nc4)cc3)cs2)n1] | IND- 0116044 | 387.5031 | CN(C)C4═CC═C (C═N4)C3═CC═C (C═C3)C2═CSC (═N2)NC1═CC═CC (═N1)C | 77.1 | 14.3 |
| 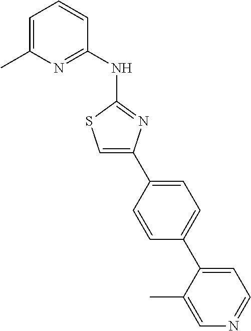 [SMILES: Cc1cccc(Nc2nc(-c3ccc(-c4ccncc4C)cc3)cs2)n1] | IND- 0116045 | 358.46166 | CC4═CC═CC (═N4)NC3═NC(═CS3) C2═CC═C (C═C2) C1═CC═NC═C1C | | |
| 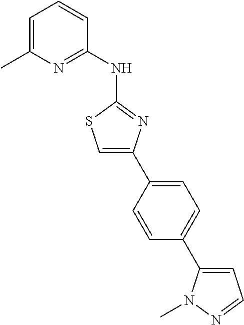 [SMILES: Cc1cccc(Nc2nc(-c3ccc(-c4ccnn4C)cc3)cs2)n1] | IND- 0116046 | 347.439 | CC4═CC═CC (═N4)NC3═NC(═CS3) C2═CC═C (C═C2) C1═CC═NN1C | | |
| 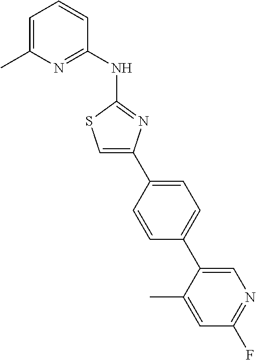 [SMILES: Cc1cccc(Nc2nc(-c3ccc(-c4cnc(F)cc4C)cc3)cs2)n1] | IND- 0116047 | 376.45206 | CC4═CC═CC (═N4)NC3═NC(═CS3) C2═CC═C (C═C2) C1═CN═C(F)C═C1C | | |
| 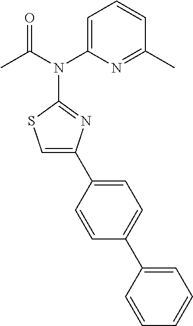 [SMILES: CC(=O)N(c1cccc(C)n1)c1nc(-c2ccc(-c3ccccc3)cc2)cs1] | IND- 0116048 | 385.48372 | CC(═O)N(C3═NC (═CS3)C2═CC═C (C═C2) C1═CC═CC═C1) C4═CC═CC (═N4)C | | |
| 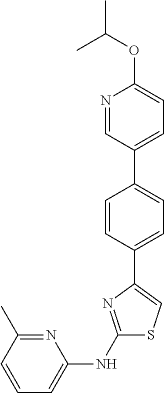 [SMILES: Cc1cccc(Nc2nc(-c3ccc(-c4ccc(OC(C)C)nc4)cc3)cs2)n1] | IND- 0116091 | 402.51446 | CC(C)OC4═CC═C (C═N4)C3═CC═C (C═C3) C2═CSC(═N2) NC1═CC═CC (═N1)C | 13.1 | 3.2 |
| 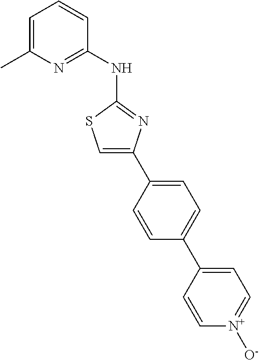 [SMILES: Cc1cccc(Nc2nc(-c3ccc(-c4cc[n+]([O-])cc4)cc3)cs2)n1] | IND- 0116092 | 360.43436 | CC4═CC═CC (═N4)NC3═NC(═CS3) C2═CC═C(C═C2) C1═CC═[N+]([O−]) C═C1 | 17.6 | 15.2 |
| 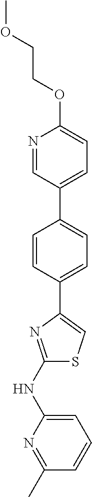 [SMILES: COCCOc1ccc(-c2ccc(-c3csc(Nc4cccc(C)n4)n3)cc2)cn1] | IND- 0116094 | 418.51386 | COCCOC4═CC═C (C═N4)C3═CC═C (C═C3)C2═CSC (═N2)NC1═CC═CC (═N1)C | 62.8 | 2.2 |
| 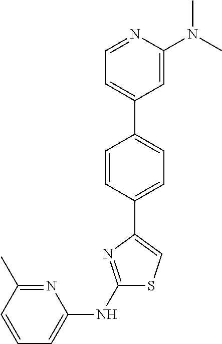 [SMILES: Cc1cccc(Nc2nc(-c3ccc(-c4ccnc(N(C)C)c4)cc3)cs2)n1] | IND- 0116095 | 387.5031 | CN(C)C4═CC (═CC═N4) C3═CC═C (C═C3)C2═CSC (═N2)NC1═CC═CC (═N1)C | >150 | 1.6 |
| 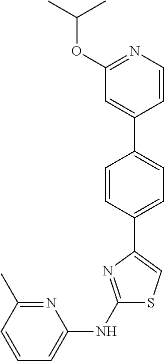 [SMILES: Cc1cccc(Nc2nc(-c3ccc(-c4ccnc(OC(C)C)c4)cc3)cs2)n1] | IND- 0116096 | 402.51446 | CC(C)OC4═NC═CC (═C4)C3═CC═C (C═C3)C2═CSC (═N2)NC1═CC═CC (═N1)C | >150 | 3.1 |
| 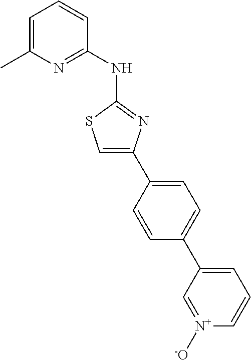 | IND- 0116097 | 360.43436 | CC4═CC═CC (═N4)NC3═NC(═CS3) C2═CC═C (C═C2)C1═CC═C [N+](═C1)[O−] | 49 | 5.1 |
| 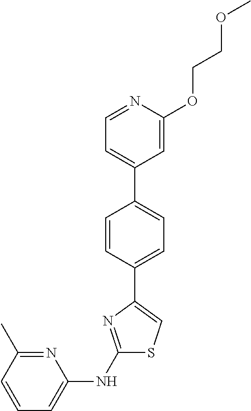 [SMILES: COCCOc1cc(-c2ccc(-c3csc(Nc4cccc(C)n4)n3)cc2)ccn1] | IND- 0116099 | 418.51386 | COCCOC4═CC (═CC═N4)C3═CC═C (C═C3)C2═CSC (═N2)NC1═CC═CC (═N1)C | 125.5 | <0.5 |
| 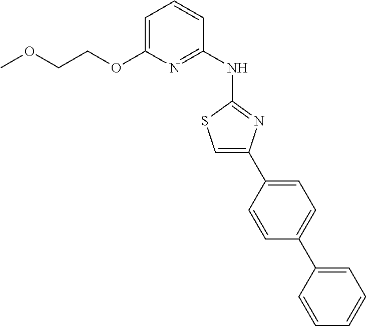 [SMILES: COCCOc1cccc(Nc2nc(-c3ccc(-c4ccccc4)cc3)cs2)n1] | IND- 0116102 | 403.49912 | COCCOC4═CC═CC (═N4)NC3═NC (═CS3)C2═CC═C (C═C2) C1═CC═CC═C1 | 2.1 | 2.7 |
| 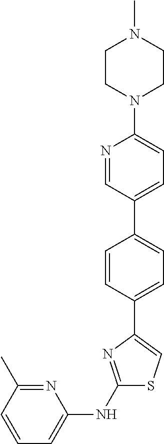 [SMILES: Cc1cccc(Nc2nc(-c3ccc(-c4ccc(N5CCN(C)CC5)nc4)cc3)cs2)n1] | IND- 0116103 | 442.58194 | CN5CCN (CC5)C4═CC═C (C═N4)C3═CC═C (C═C3)C2═CS C(═N2) NC1═CC═CC (═N1)C | 37.1 | 4.4 |
| 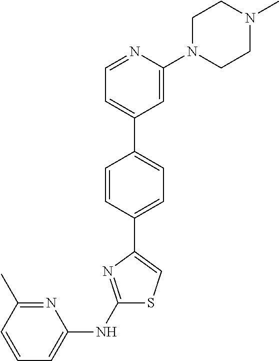 [SMILES: Cc1cccc(Nc2nc(-c3ccc(-c4ccnc(N5CCN(C)CC5)c4)cc3)cs2)n1] | IND- 0116134 | 442.58194 | CN5CCN (CC5)C4═CC (═CC═N4) C3═CC═C (C═C3)C2═CSC (═N2) NC1═CC═CC (═N1)C | 81.8 | 40.2 |
| 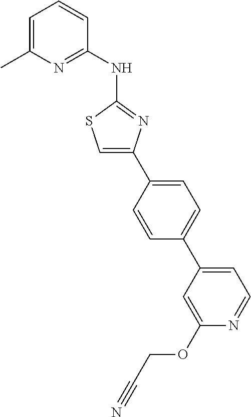 [SMILES: Cc1cccc(Nc2nc(-c3ccc(-c4ccnc(OCC#N)c4)cc3)cs2)n1] | IND- 0116138 | 399.4705 | CC4═CC═CC (═N4) NC3═NC(═CS3) C2═CC═C (C═C2) C1═CC═NC (═C1)OCC#N | >150 | <0.4 |
| 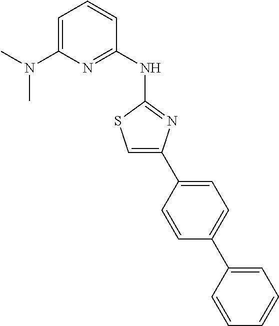 [SMILES: CN(C)c1cccc(Nc2nc(-c3ccc(-c4ccccc4)cc3)cs2)n1] | IND- 0116142 | 372.48836 | CN(C)C4═CC═CC (═N4) NC3═NC(═CS3) C2═CC═C (C═C2) C1═CC═CC═C1 | 16.1 | <0.5 |
| 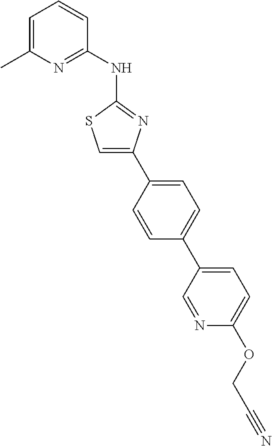 [SMILES: Cc1cccc(Nc2nc(-c3ccc(-c4ccc(OCC#N)nc4)cc3)cs2)n1] | IND- 0116165 | 399.4705 | CC4═CC═CC (═N4) NC3═NC(═CS3) C2═CC═C (C═C2) C1═CC═C (OCC#N)N═C1 | 2.2 | <0.4 |
| 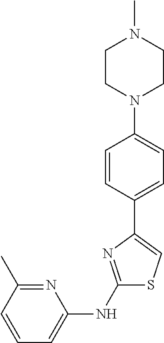 [SMILES: Cc1cccc(Nc2nc(-c3ccc(N4CCN(C)CC4)cc3)cs2)n1] | IND- 0126259 | 365.4977 | CN4CCN(CC4) C3═CC═C(C═C3) C2═CSC(═N2) NC1═CC═CC (═N1)C | | |
| 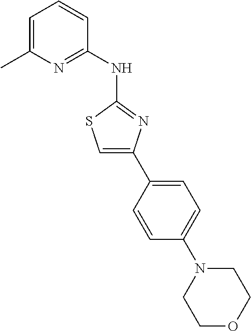 [SMILES: Cc1cccc(Nc2nc(-c3ccc(N4CCOCC4)cc3)cs2)n1] | IND- 0126260 | 352.45566 | CC4═CC═CC (═N4) NC3═NC(═CS3) C2═CC═C(C═C2) N1C COCC1 | | |
| 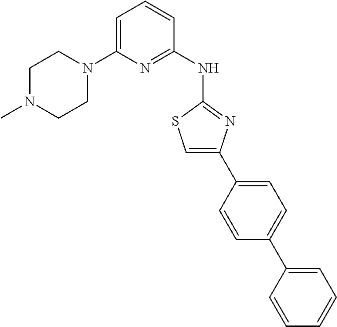 [SMILES: CN1CCN(c2cccc(Nc3nc(-c4ccc(-c5ccccc5)cc4)cs3)n2)CC1] | IND- 0126437 | 427.5672 | CN5CCN(CC5) C4═CC═CC (═N4) NC3═NC(═CS3) C2═CC═C(C═C2) C1═CC═CC═C1 | | |

TABLE 21
In vitro microsome stability, permeability data, and calculated physiochemical
properties for select 2-aminothiazole analogs.

| | Antiprion | | | | | | | |
|---|---|---|---|---|---|---|---|---|
| | activity | Microsome | Permeability^{b} | | | | | |
| | ScN2a-cl3 | stability^{a} | (10⁻⁶ cm/sec) | | Calculated properties^{c} | | | |
| Compound | EC₅₀ (μM) | T_{1/2} (min) | P_{A}_{_{→}}_{B} | P_{B}_{_{→}}_{A} | MW | PSA (Å²) | ClogP | HBD |
| 25 | 7.88 | 10 | n.d | n.d | 267.4 | 66.0 | 4.5 | 1 |
| 23 | 1.57 | 20 | 32.6 | 42.3 | 268.3 | 78.9 | 3.2 | 1 |
| 27 | 0.94 | 151 | 6.8 | 8.3 | 334.4 | 92.1 | 4.9 | 1 |
| 28 | >26 | 19 | 11.2 | 8.6 | 348.4 | 92.1 | 5.1 | 1 |
| 12 | 2.53 | 150 | 9.4 | 8.7 | 381.4 | 84.5 | 4.4 | 1 |
| 13 | 1.00 | 45 | 20.1 | 24.4 | 343.4 | 93.7 | 3.3 | 1 |
| 17 | 0.11 | 83 | 13.2 | 10.5 | 363.4 | 84.5 | 4.5 | 1 |
| 18 | 0.43 | 69 | 9.2 | 8.9 | 363.4 | 84.5 | 4.9 | 1 |

^{a}rat liver microsomes.
^{b}Permeability across MDR1-MDCK cell monolayers in the apical to basal (absorptive) and basal to apical (secretory) directions.
^{c}Molecular Weight (MW), Polar Surface Area (PSA) and calculated n-octanol water partition coefficient (ClogP) values calculated using MarvinSketch 4.1.8.
HBD = number of hydrogen bond donors.
n.d. = not determined.

TABLE 22
Table of EC₅₀ and pEC₅₀ values (three separate determinations and corresponding means)
for compounds 3-50 in the ELISA ScN2a-cl3 dividing cell assay; standard deviation and coefficient
of variance values calculated from pEC₅₀ values. Table of EC₅₀ values (means of three
determinations) for compounds 3-50 in the ELISA ScN2a-cl3 non-dividing cell assay
and the calcein-AM assay (cell viability assay).

| | Dividing | | | | Dividing | | | | | |
|---|---|---|---|---|---|---|---|---|---|---|
| | ELISA EC₅₀ (μM) | | | | ELISA pEC₅₀ (μM) | | | | | |
| Compound | 1st Expt. | 2nd Expt. | 3rd Expt. | Mean | 1st Expt. | 2nd Expt. | 3rd Expt. | Mean | SD | CV (%) |
| 3 | 1.4 | 2.774 | 9.249 | 4.474 | 5.85 | 5.56 | 5.03 | 5.48 | 0.42 | 8 |
| 4 | 5.618 | 1.194 | 2.218 | 3.010 | 5.25 | 5.92 | 5.65 | 5.61 | 0.34 | 6 |
| 5 | >32 | >32 | >32 | >32 | >4.5 | >4.5 | >4.5 | | | |
| 6 | 11.8 | 6.474 | 6.384 | 8.219 | 4.93 | 5.19 | 5.19 | 5.10 | 0.15 | 3 |
| 7 | 1.413 | 1.159 | 1.097 | 1.223 | 5.85 | 5.94 | 5.96 | 5.92 | 0.06 | 1 |
| 8 | >32 | 25.2 | 26.9 | >28 | >4.5 | 4.60 | 4.57 | | | |
| 9 | 7.402 | 7.295 | 4.445 | 6.381 | 5.13 | 5.14 | 5.35 | 5.21 | 0.13 | 2 |
| 10 | 3.254 | 6.706 | 1.85 | 3.937 | 5.49 | 5.17 | 5.73 | 5.46 | 0.28 | 5 |
| 11 | 26 | 9.233 | 11.7 | 15.644 | 4.59 | 5.03 | 4.93 | 4.85 | 0.24 | 5 |
| 12 | 2.93 | 2.048 | 2.617 | 2.532 | 5.53 | 5.69 | 5.58 | 5.60 | 0.08 | 1 |
| 13 | 1.073 | 1.004 | 0.917 | 0.998 | 5.97 | 6.00 | 6.04 | 6.00 | 0.03 | 1 |
| 14 | 0.974 | 0.577 | 0.815 | 0.789 | 6.01 | 6.24 | 6.09 | 6.11 | 0.12 | 2 |
| 15 | 13 | 2.067 | 6.809 | 7.292 | 4.89 | 5.68 | 5.17 | 5.25 | 0.41 | 8 |
| 16 | 1.134 | 0.736 | 1.131 | 1.000 | 5.95 | 6.13 | 5.95 | 6.01 | 0.11 | 2 |
| 17 | 0.101 | 0.117 | 0.108 | 0.109 | 7.00 | 6.93 | 6.97 | 6.96 | 0.03 | 0 |
| 18 | 0.486 | 0.34 | 0.453 | 0.426 | 6.31 | 6.47 | 6.34 | 6.38 | 0.08 | 1 |
| 19 | 0.436 | 0.317 | 0.413 | 0.389 | 6.36 | 6.50 | 6.38 | 6.41 | 0.07 | 1 |
| 20 | >32 | >32 | >32 | >32 | >4.5 | >4.5 | >4.5 | | | |
| 21 | 5.016 | 1.889 | 2.187 | 3.031 | 5.30 | 5.72 | 5.66 | 5.56 | 0.23 | 4 |
| 22 | >32 | 26.1 | 30.2 | >29 | >4.5 | 4.58 | 4.52 | | | |
| 23 | 1.119 | 1.746 | 1.85 | 1.572 | 5.95 | 5.76 | 5.73 | 5.81 | 0.12 | 2 |
| 24 | 1.624 | 0.507 | 0.449 | 0.860 | 5.79 | 6.29 | 6.35 | 6.14 | 0.31 | 5 |
| 25 | 13 | 4.9 | 5.735 | 7.878 | 4.89 | 5.31 | 5.24 | 5.15 | 0.23 | 4 |
| 26 | 1.578 | 0.879 | 1.22 | 1.226 | 5.80 | 6.06 | 5.91 | 5.92 | 0.13 | 2 |
| 27 | 1.093 | 0.747 | 0.994 | 0.945 | 5.96 | 6.13 | 6.00 | 6.03 | 0.09 | 1 |
| 28 | 30 | 15.2 | >32 | >26 | 4.52 | 4.82 | >4.5 | | | |
| 29 | >32 | >32 | >32 | >32 | >4.5 | >4.5 | >4.5 | | | |
| 30 | 0.226 | 0.385 | 0.414 | 0.342 | 6.65 | 6.41 | 6.38 | 6.48 | 0.14 | 2 |
| 31 | >32 | >32 | >32 | >32 | >4.5 | >4.5 | >4.5 | | | |
| 32 | 0.261 | 0.277 | 0.383 | 0.307 | 6.58 | 6.56 | 6.42 | 6.52 | 0.09 | 1 |
| 33 | 2.062 | 2.849 | 4.315 | 3.075 | 5.69 | 5.55 | 5.37 | 5.53 | 0.16 | 3 |
| 34 | 2.248 | 1.609 | 2.16 | 2.006 | 5.65 | 5.79 | 5.67 | 5.70 | 0.08 | 1 |
| 35 | 1.162 | 0.573 | 0.663 | 0.799 | 5.93 | 6.24 | 6.18 | 6.12 | 0.16 | 3 |
| 36 | >32 | >32 | >32 | >32 | >4.5 | >4.5 | >4.5 | | | |
| 37 | 8.999 | 7.257 | 9.711 | 8.656 | 5.05 | 5.14 | 5.01 | 5.07 | 0.07 | 1 |
| 38 | 1.989 | 2.993 | 2.339 | 2.440 | 5.70 | 5.52 | 5.63 | 5.62 | 0.09 | 2 |
| 39 | 3.454 | 5.926 | 7.382 | 5.587 | 5.46 | 5.23 | 5.13 | 5.27 | 0.17 | 3 |
| 40 | 0.395 | 0.11 | 0.183 | 0.229 | 6.40 | 6.96 | 6.74 | 6.70 | 0.28 | 4 |
| 41 | 0.369 | 0.166 | 0.219 | 0.251 | 6.43 | 6.78 | 6.66 | 6.62 | 0.18 | 3 |
| 42 | 0.629 | 0.599 | 0.585 | 0.604 | 6.20 | 6.22 | 6.23 | 6.22 | 0.02 | 0 |
| 43 | 1.054 | 1.1 | 1.671 | 1.275 | 5.98 | 5.96 | 5.78 | 5.90 | 0.11 | 2 |
| 44 | 0.926 | 0.733 | 0.794 | 0.818 | 6.03 | 6.13 | 6.10 | 6.09 | 0.05 | 1 |
| 45 | 3.161 | 3.254 | 6.833 | 4.416 | 5.50 | 5.49 | 5.17 | 5.38 | 0.19 | 4 |
| 46 | 3.194 | 1.927 | 2.205 | 2.442 | 5.50 | 5.72 | 5.66 | 5.62 | 0.11 | 2 |
| 47 | 9.812 | 3.932 | 10.8 | 8.181 | 5.01 | 5.41 | 4.97 | 5.13 | 0.24 | 5 |
| 48 | >10 | >10 | >10 | >10 | >4.5 | >4.5 | >4.5 | | | |
| 49 | 0.152 | 0.196 | 0.083 | 0.144 | 6.82 | 6.71 | 7.08 | 6.87 | 0.19 | 3 |
| 50 | 0.091 | 0.089 | 0.065 | 0.082 | 7.04 | 7.05 | 7.19 | 7.09 | 0.08 | 1 |

TABLE 23
Table of EC₅₀ and pEC₅₀ values (three separate determinations and
corresponding means) for compounds 3-50 in the ELISA ScN2a-c13
dividing cell assay; standard deviation and coefficient of variance
values calculated from pEC₅₀ values. Table of EC₅₀ values (means of
three determinations) for compounds 3-50 in the ELISA ScN2a-c13
non-dividing cell assay and the calcein-AM assay (cell viability assay).

| | Dividing | Non-Dividing |
|---|---|---|
| | Calcein EC₅₀ (μM) | ELISA EC₅₀ (μM) |
| Compound | Mean (n = 3) | Mean (n = 3) |
| 3 | >10 | >10 |
| 4 | >32 | >32 |
| 5 | >32 | >32 |
| 6 | >32 | >32 |
| 7 | >32 | >32 |
| 8 | >32 | >32 |
| 9 | >32 | >32 |
| 10 | >32 | >32 |
| 11 | >32 | >32 |
| 12 | >32 | >32 |
| 13 | >32 | >32 |
| 14 | >32 | >32 |
| 15 | >32 | >32 |
| 16 | >32 | >32 |
| 17 | >32 | >32 |
| 18 | >32 | >32 |
| 19 | >32 | >32 |
| 20 | >32 | >32 |
| 21 | >32 | >32 |
| 22 | >32 | >32 |
| 23 | >32 | >32 |
| 24 | 0.389 | >32 |
| 25 | >32 | >32 |
| 26 | >32 | >32 |
| 27 | >32 | >32 |
| 28 | >32 | >32 |
| 29 | >32 | >32 |
| 30 | >32 | >32 |
| 31 | >32 | >32 |
| 32 | >32 | >32 |
| 33 | >32 | >32 |
| 34 | >32 | >32 |
| 35 | >32 | >32 |
| 36 | >32 | >32 |
| 37 | >32 | >32 |
| 38 | >32 | >32 |
| 39 | >32 | >32 |
| 40 | >32 | >32 |
| 41 | >32 | >32 |
| 42 | >32 | >32 |
| 43 | >32 | >32 |
| 44 | >32 | >32 |
| 45 | >32 | >32 |
| 46 | >32 | >32 |
| 47 | >10 | >10 |
| 48 | >10 | >10 |
| 49 | >10 | >10 |
| 50 | >10 | >10 |

### Y. Antiprion Activity and Cell Viability Effects

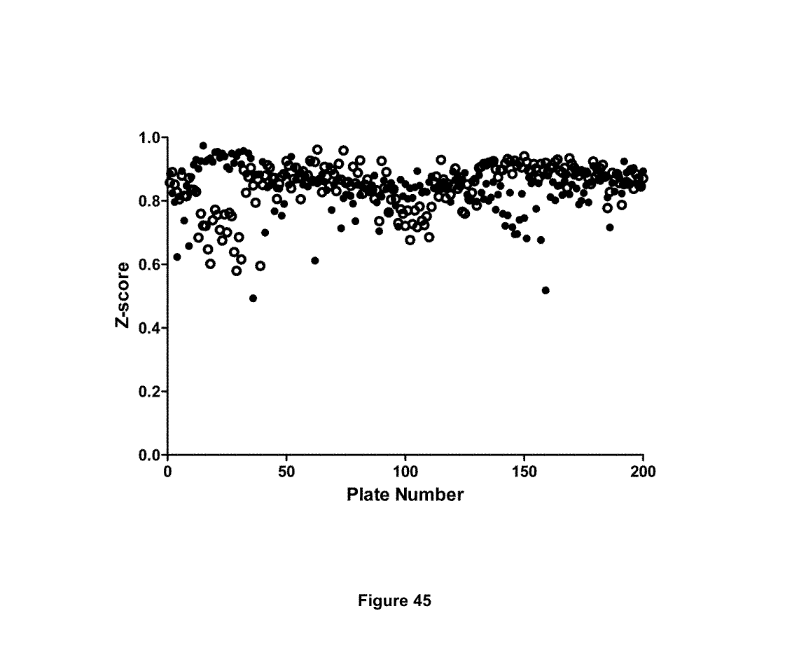

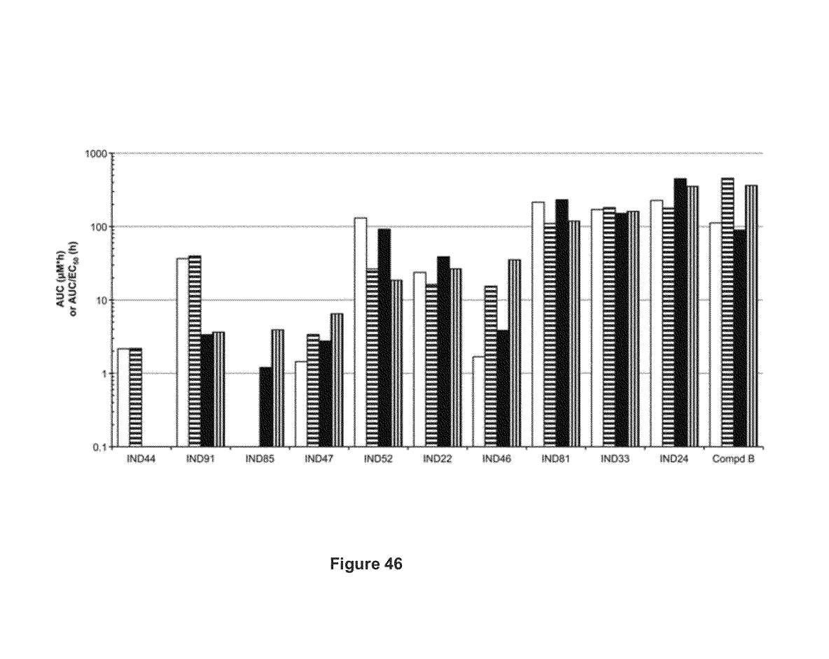

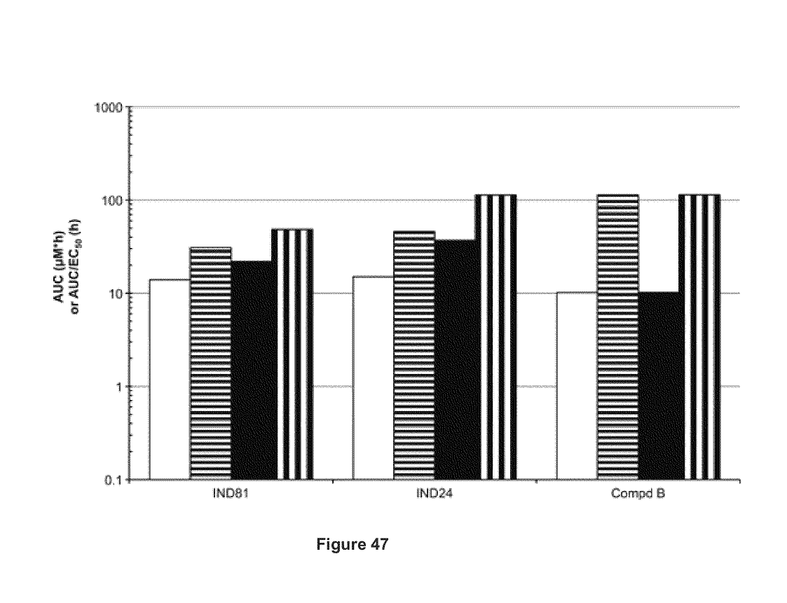

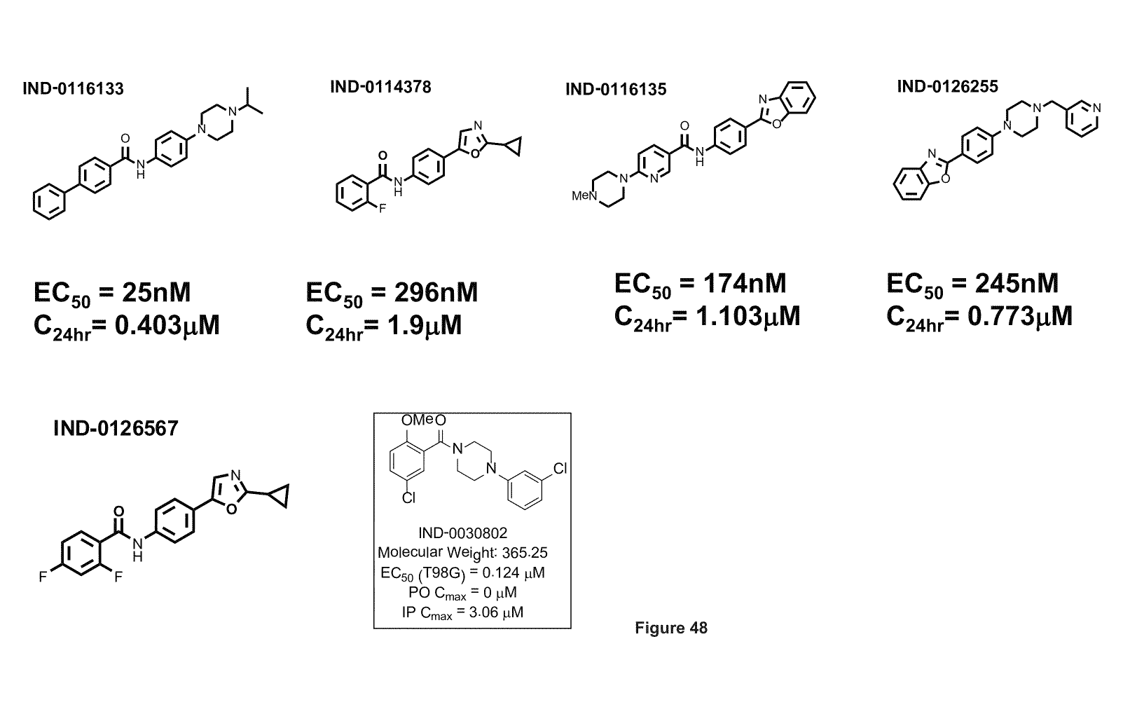

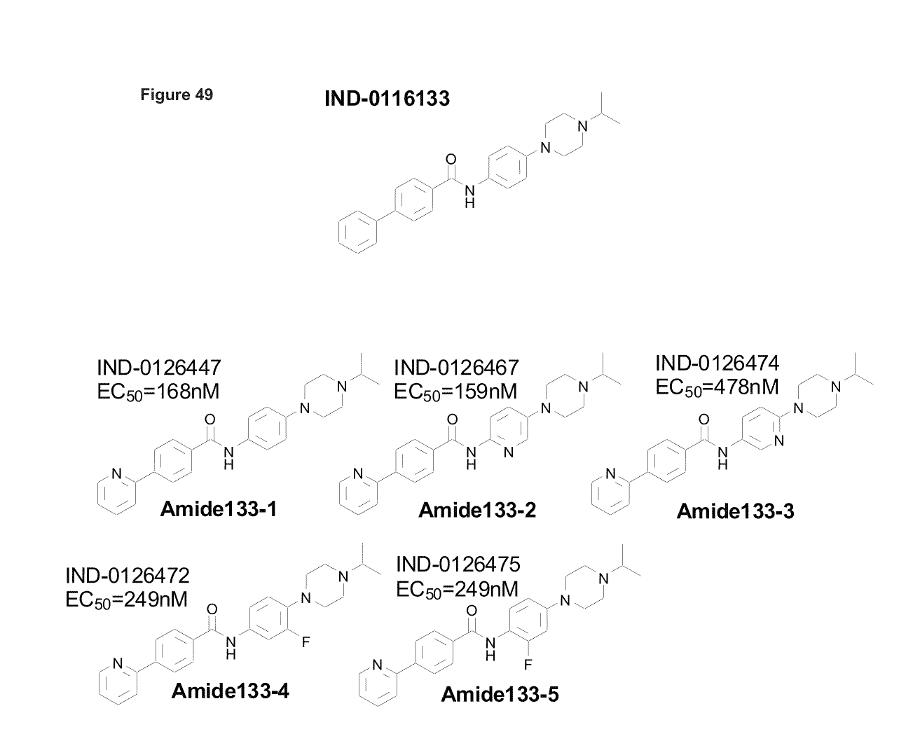

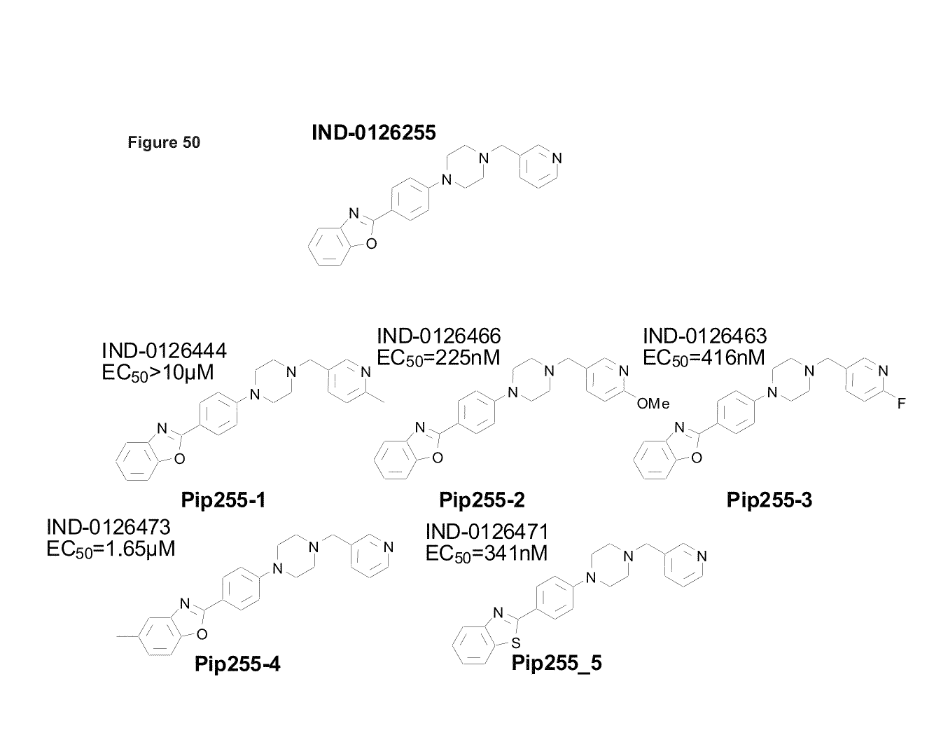

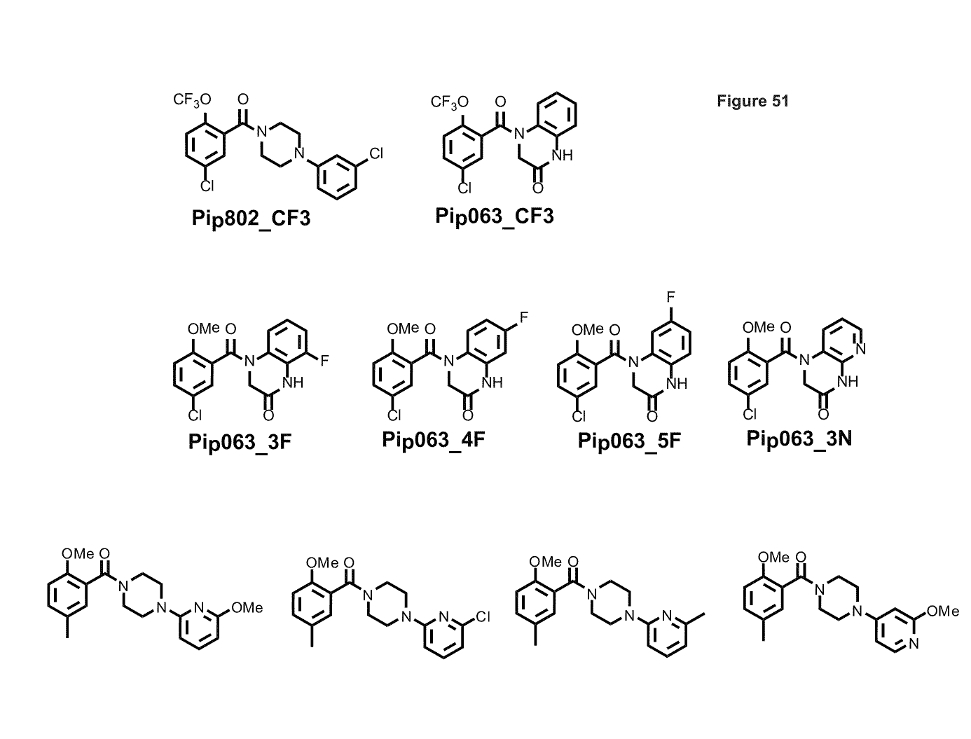

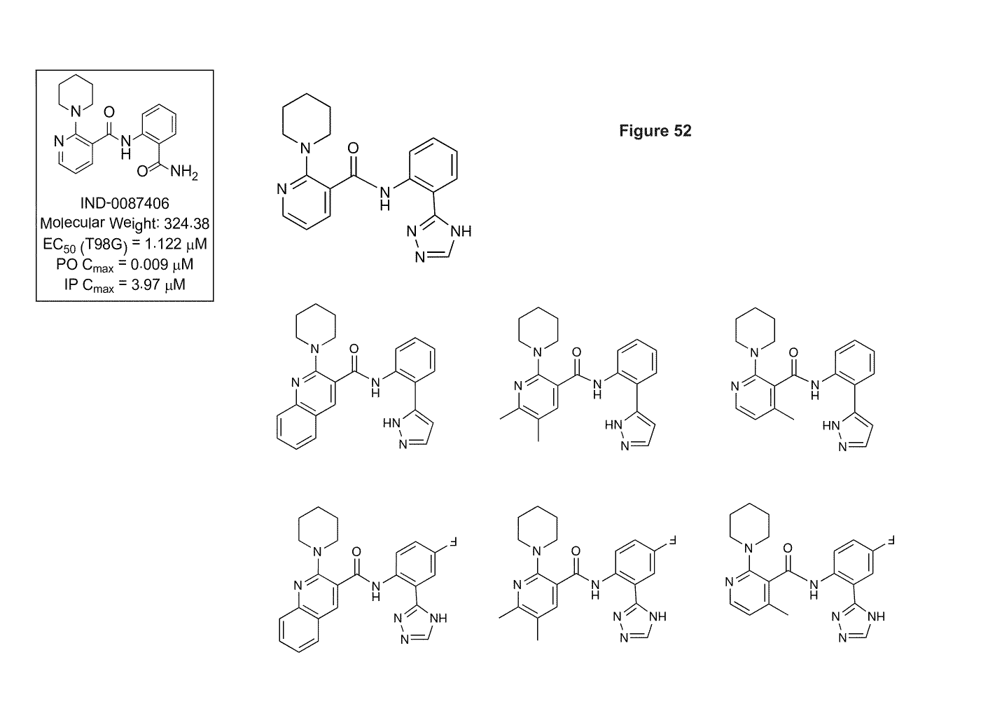

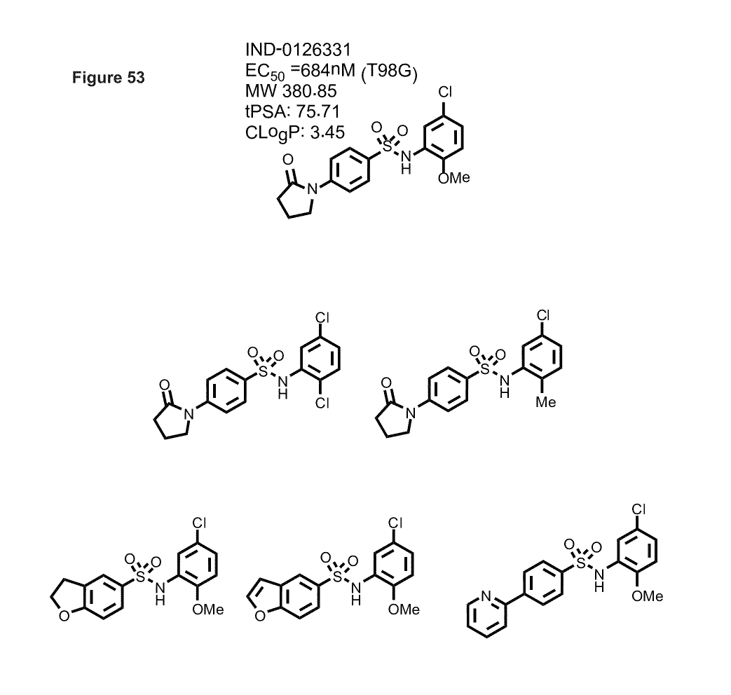

To establish the antiprion activity and cell viability effects of the commercial compounds, dose-response curves were used to calculate EC₅₀ in ELISA and cell viability assays in human T98G glioblastoma and human IMR32 neuroblastoma cells. Z-scores for 190 runs was excellent in both cells lines, ranging from 0.6-0.95 (FIG. 45).

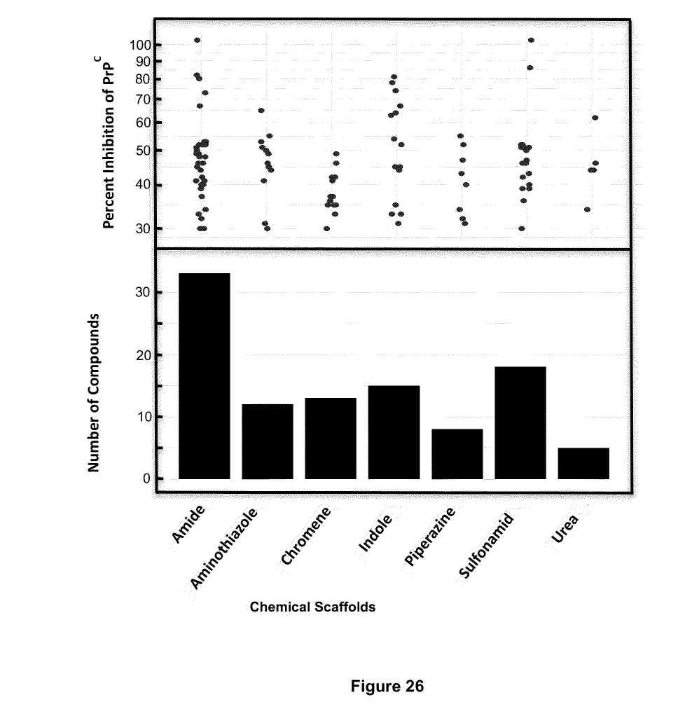

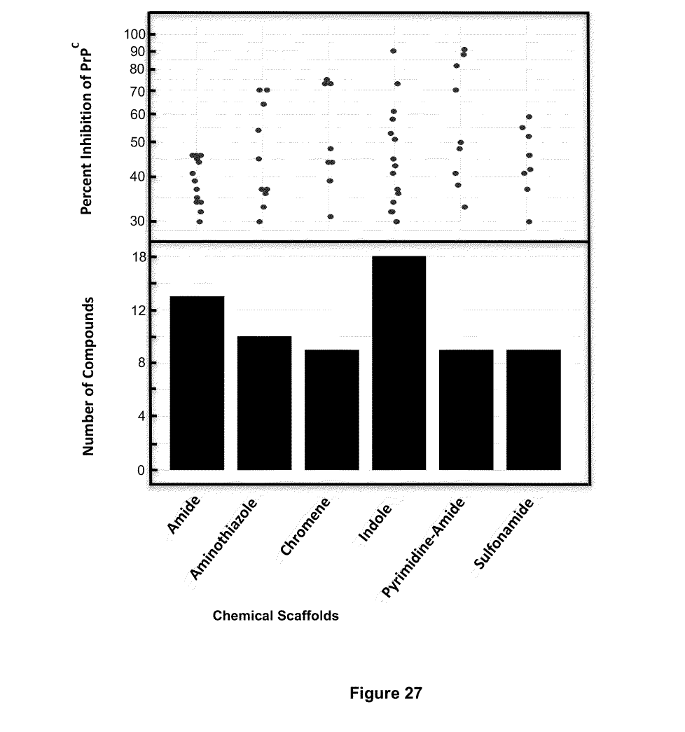

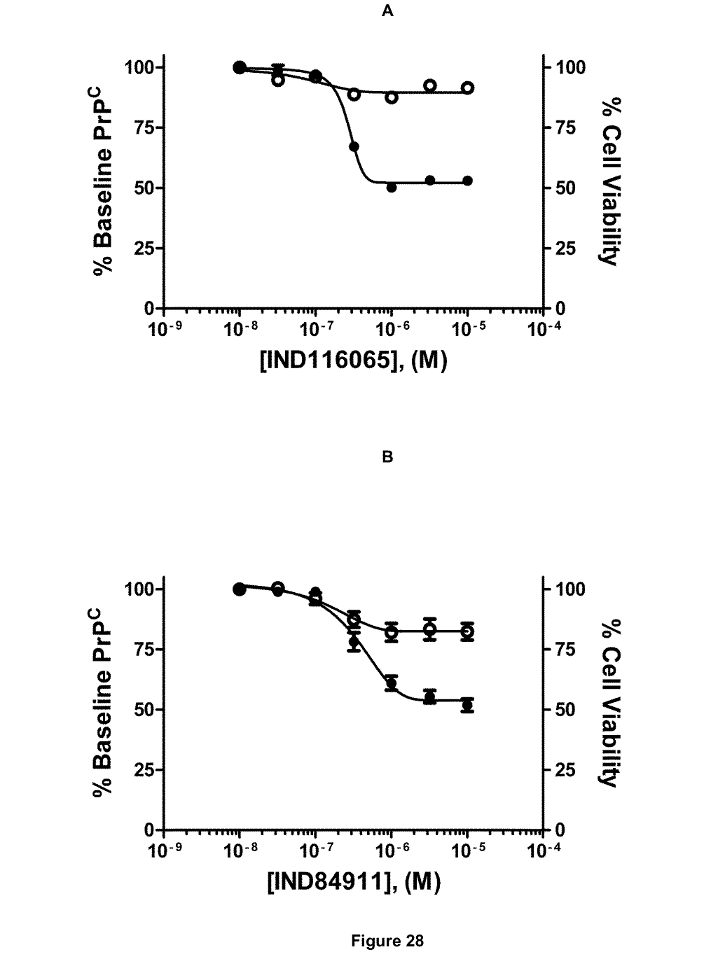

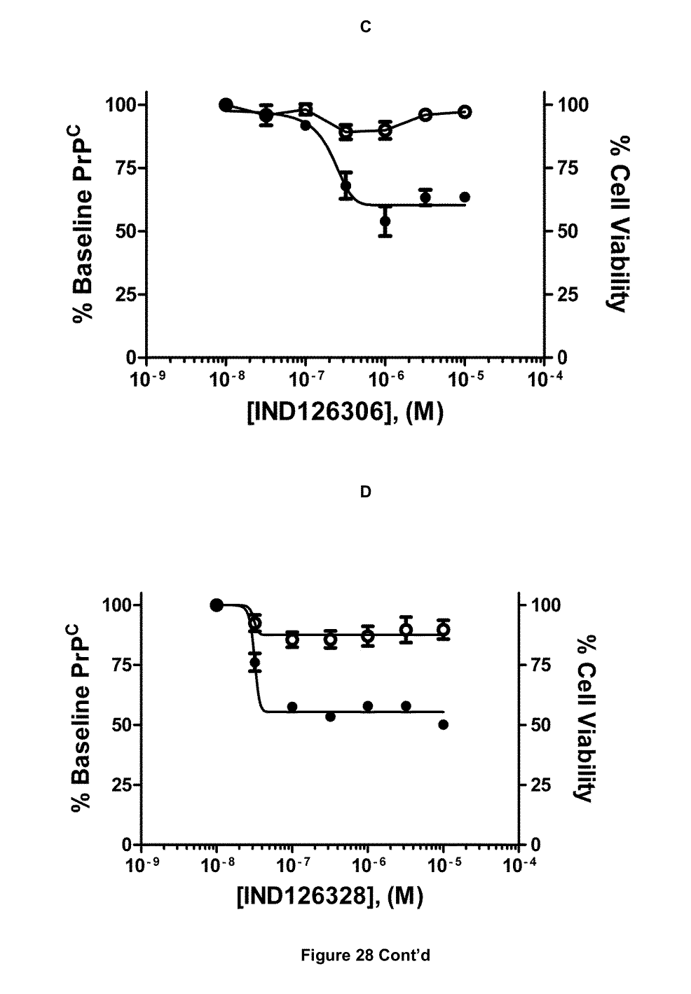

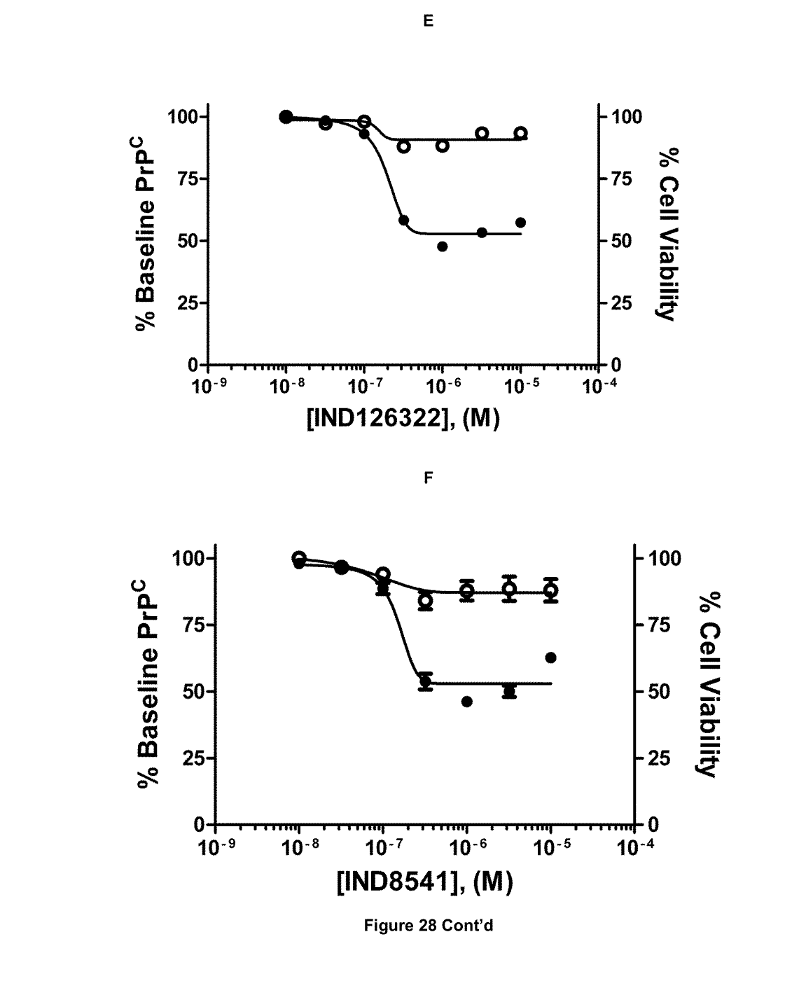

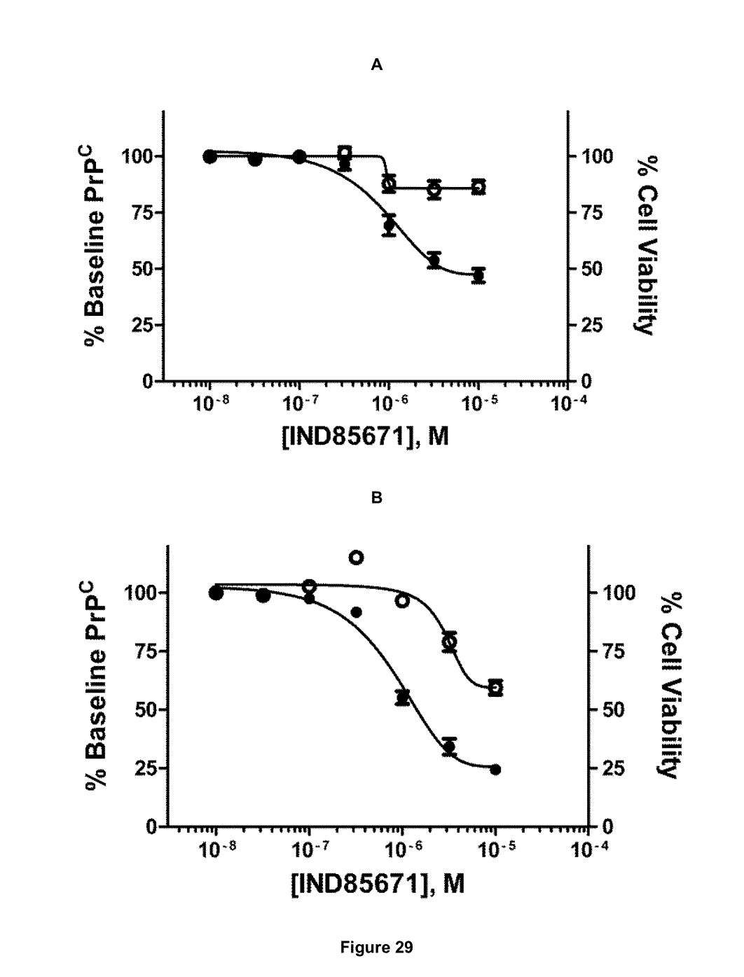

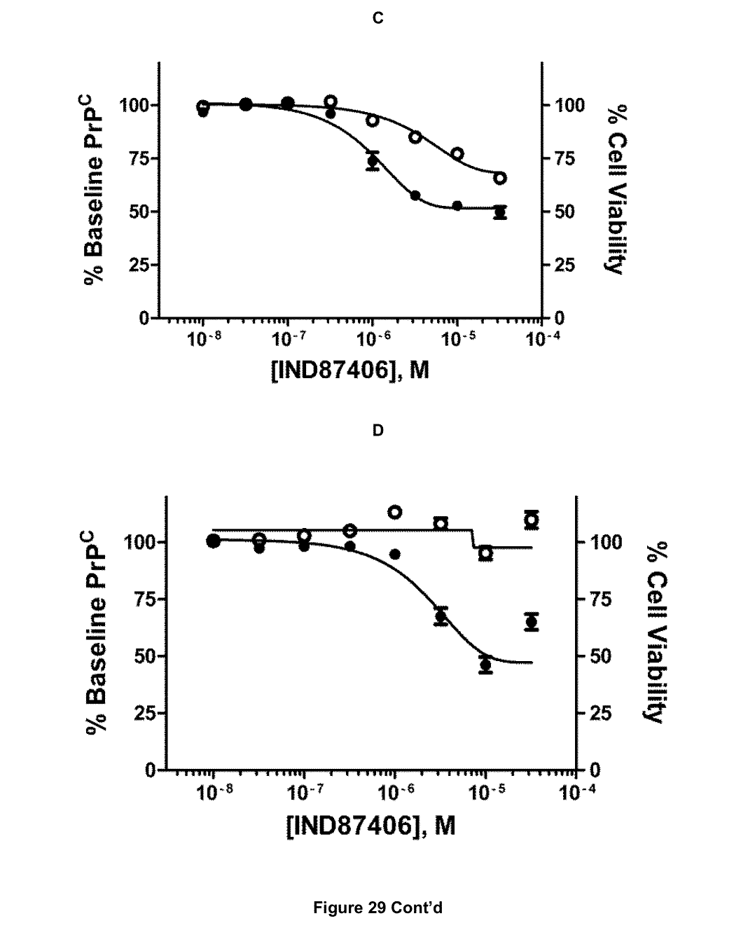

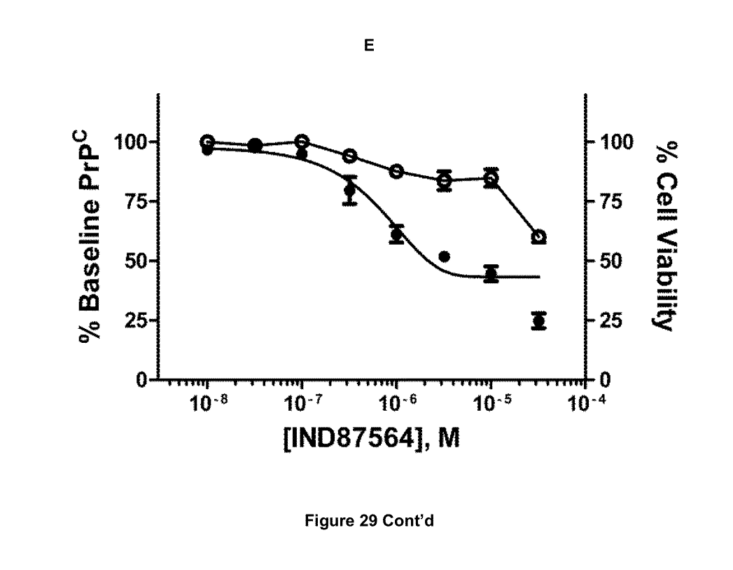

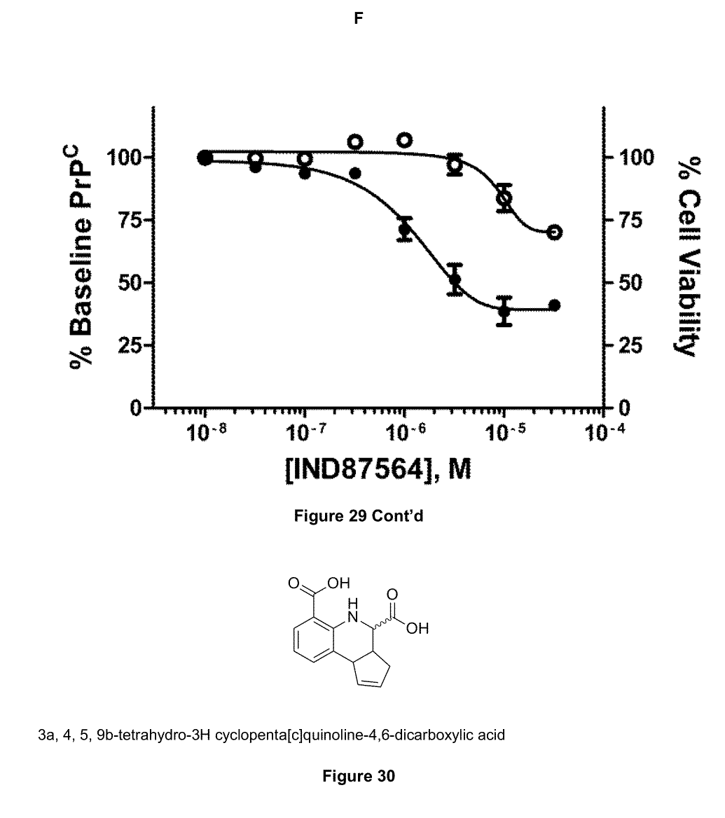

For T98G and IMR32 cells, 579 and 675 HTS hits, respectively, were identified. SPC assays were performed on these HTS hits, and 138 and 114 confirmed SPC hits were found for T98G and IMR32 cells, respectively. Confirmed SPC hits led to the initial identification of 7 chemical scaffolds for T98G cells (FIG. 26) and 6 chemical scaffolds for IMR32 cells (FIG. 27), 5 of which were active in both cell lines. Over 300 confirmed SPC hits and related analogs were tested in T98G and IMR32 cells for potency (EC₅₀) in ELISA and calcein (cell viability) assays by generating dose-titration curves in both cell lines. The distribution of potency for the six scaffolds resulting from T98G cells is shown (Table 24). The potency (EC₅₀) values in the PrP^{C} ELISA and calcein assays in T98G cells along with calculated physicochemical properties are provided for the 32 confirmed SPC hits, representing the six chemical scaffolds (Table 25). These showed potency (EC₅₀=0.065 4.1 μM) and none affected cell viability (EC₅₀>10 μM). EC₅₀ ELISA and calcein curves for two of the potent confirmed hits from three representative scaffolds are shown (FIG. 28). Twenty-nine of the 32 that had good to excellent potency in T98G cells were also tested in N2a-c13 cells. Three showed potency and no effect on cell viability (FIG. 29). All confirmed SPC hits tested for potency in IMR32 cells were either inactive (EC₅₀>10 μM) or active (EC₅₀<1 or between 1-10 μM) at a concentration that also affected cell viability (data not shown) and were not followed up further. Additionally, 29 of the 32 confirmed SPC hits (Table 25) were tested in mouse N2a-c13 cells and three of these compounds showed significant reduction of PrP^{C} with little or no effect on cell viability (FIG. 29).

To seed plates for compound treatment, the growth medium (supplemented MEM) was aspirated from the flasks, the cells washed twice with 10 mL of calcium- and magnesium-free Dulbecco's PBS, and then detached by addition of 3 mL of Cell Dissociation Buffer after incubation at RT for 5 min. The dissociation buffer was aspirated and the cells suspended in 10 mL of growth medium before counting using a Cellometer Auto T4 (Nexcelom Biosciences; Lawrence, Mass.). Each well of a white, clear-bottom, 96-well plate (Greiner) was seeded with 10,000 cells using a Matrix Wellmate dispenser and allowed to incubate overnight at 37° C. The next day, test compounds were added to each well and the plates returned to the incubator. After 2 days, the growth medium was aspirated, and each well washed once with PBS supplemented with 0.25 mg/mL BSA (wash buffer) and aspirated dry. IMR32 and N2a cells were fixed by the addition of 50 μL/well of 4% paraformaldehyde (in PBS); T98G cells were used without fixation by paraformaldehyde. After 20 min at room temperature (RT), the paraformaldehyde was removed by three washes of 250 μL/well of PBS and the wells aspirated dry. Horseradish peroxidase (HRP) conjugated anti-human PrP^{C} P antibody (Williamson et al., 1998) (100 μL of a 1:1000 dilution in PBS supplemented with 3% w/v nonfat milk) was added to each well and the plate incubated at RT for 1 h. The antibody was removed with 5-6 washes of buffer (300 μL/well/wash), then 50 μL of Supersignal ELISA Pico Chemiluminescent substrate (Pierce Thermo) added to each well and the luminescence at 425 nm read immediately using a Spectramax M5 plate reader.

### Hepatic Microsomal Stability

Stock solutions of the compounds (0.5 mM) were prepared in DMSO. These were diluted 500-fold into 1 mL of microsomal incubation mixture to yield a final concentration of 1 μM. The incubation mixture was composed of 100 mM phosphate buffer, pH 7.4, and NADPH regenerating system (BD Biosciences NADPH Regenerating System Solutions A & B). This mixture was preincubated at 37° C. for 5 min in an Eppendorf Thermo mixer, and the reaction initiated by addition of 0.5 mg (25 μl of a 20 mg/mL solution) of liver microsomes. Aliquots (50 μl) were withdrawn at 0, 5, 15, 30, and 60 min, and added to 100 μl acetonitrile containing internal standard.

After centrifugation at ˜12,000×g for 10 min, the supernatants were analyzed by LC/MS/MS. The percentage of solute remaining at the end of the incubation was used to determine species-specific values of scaled intrinsic clearance, hepatic clearance, and predicted hepatic extraction based upon the calculations below (Obach, 1999).

For LC/MS/MS quantification, samples and their respective internal standards were injected onto a BetaBasic C18 column maintained at room temperature and separated using a gradient between 0.1% formic acid in water and 0.1% formic acid in acetonitrile. Data acquisition used MRM in the positive ion mode using appropriate MRM transitions for each compound.

TABLE 24
Distribution of EC₅₀ values for the final six chemical scaffolds confirmed in
T98G cells, based on potency testing of confirmed SPC hits and related analogs.

| | | Compounds | Compounds with | Compounds with |
|---|---|---|---|---|
| Scaffold | Murcko Fragment | tested (N) | EC₅₀ <1 μM (N) | EC₅₀ 1-10 μM (N) |
| Amide | 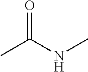 | 159 | 5 | 8 |
| Aminothiazole | 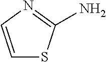 | 26 | 5 | 3 |
| Chromene | 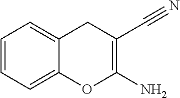 [SMILES: N#CC1=C(N)Oc2ccccc2C1] | 5 | 5 | 0 |
| Piperazine | 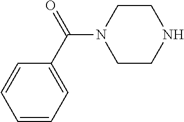 [SMILES: O=C(c1ccccc1)N1CCNCC1] | 21 | 3 | 1 |
| Fused Pyrrole | 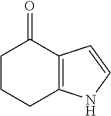 | 14 | 6 | 3 |
| Sulfonamide | 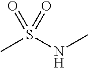 | 60 | 4 | 4 |

TABLE 25
Scaffold, compound, ELISA potency (EC₅₀), and calculated physicochemical parameters^{a} for the 31 confirmed PrP^{C}
SPC hits in T98G cells.

| | Com- | | | EC₅₀ | | | HBA/ |
|---|---|---|---|---|---|---|---|
| Scaffold | pound | Structure | MW | (μM)^{b} | tPSA | xlogP | HBD |
| Amide | IND82865 | 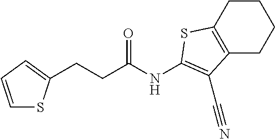 [SMILES: N#Cc1c(NC(=O)CCc2cccs2)sc2c1CCCC2] | 316.4 | 0.242* | 52.9 | 4.54 | 3/1 |
| | IND84911 | 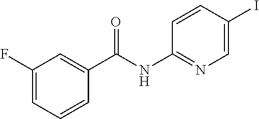 [SMILES: O=C(Nc1ccc(I)cn1)c1cccc(F)c1] | 342.1 | 0.55 | 42.0 | 3.22 | 3/1 |
| | IND116063 | 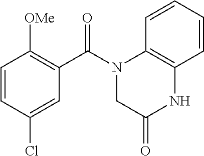 [SMILES: COc1ccc(Cl)cc1C(=O)N1CC(=O)Nc2ccccc21] | 316.7 | 0.647 | 58.6 | 2.88 | 5/1 |
| | IND85671 | 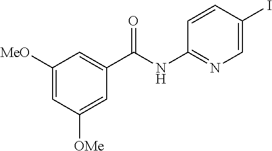 [SMILES: COc1cc(OC)cc(C(=O)Nc2ccc(I)cn2)c1] | 384.2 | 1.102 (0.863) | 60.4 | 3.04 | 5/1 |
| | IND85692 | 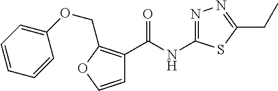 [SMILES: CCc1nnc(NC(=O)c2ccoc2COc2ccccc2)s1] | 329.4 | 2.28 | 77.6 | 4.44 | 6/1 |
| | IND87406 | 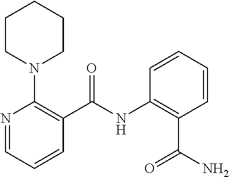 [SMILES: NC(=O)c1ccccc1NC(=O)c1cccnc1N1CCCCC1] | 324.4 | 1.122 (3.114) | 88.3 | 1.98 | 3/1 |
| | IND116065 | 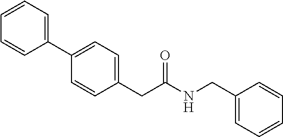 [SMILES: O=C(Cc1ccc(-c2ccccc2)cc1)NCc1ccccc1] | 301.4 | 0.233 | 29.1 | 5.05 | 2/1 |
| Amino- thiazole | IND126306 | 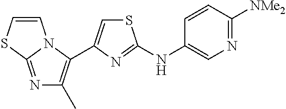 [SMILES: Cc1nc2sccn2c1-c1csc(Nc2ccc(N(C)C)nc2)n1] | 356.5 | 0.217 | 58.3 | 4.28 | 6/1 |
| | IND126328 | 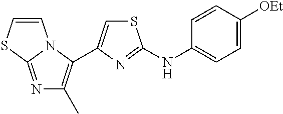 [SMILES: CCOc1ccc(Nc2nc(-c3c(C)nc4sccn34)cs2)cc1] | 356.5 | 0.065 | 51.4 | 5.59 | 5/1 |
| | IND9756 | 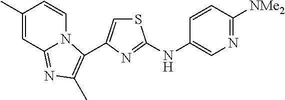 [SMILES: Cc1ccn2c(-c3csc(Nc4ccc(N(C)C)nc4)n3)c(C)nc2c1] | 363.5 | 0.18 | 45.5 | 5.29 | 5/1 |
| | IND115948 | 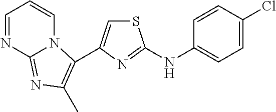 [SMILES: Cc1nc2ncccn2c1-c1csc(Nc2ccc(Cl)cc2)n1] | 341.8 | 1.85 | 55.1 | 4.49 | 5/1 |
| Chromene | IND8541 | 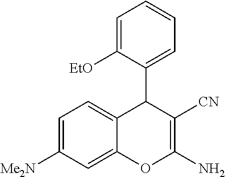 [SMILES: CCOc1ccccc1C1C(C#N)=C(N)Oc2cc(N(C)C)ccc21] | 335.4 | 0.155* | 71.5 | 3.37 | 5/1 |
| | IND17990 | 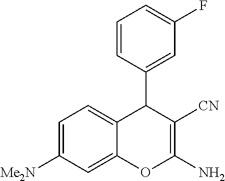 [SMILES: CN(C)c1ccc2c(c1)OC(N)=C(C#N)C2c1cccc(F)c1] | 309.3 | 0.031 | 62.3 | 3.1 | 4/1 |
| | IND126322 | 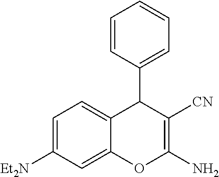 | 319.4 | 0.174 | 62.3 | 3.82 | 4/1 |
| | IND126361 | 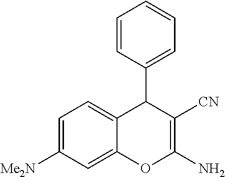 [SMILES: CN(C)c1ccc2c(c1)OC(N)=C(C#N)C2c1ccccc1] | 291.4 | 0.124 | 62.3 | 3 | 4/1 |
| | IND23846 | 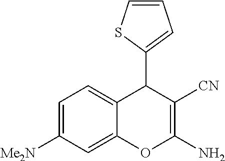 [SMILES: CN(C)c1ccc2c(c1)OC(N)=C(C#N)C2c1cccs1] | 297.4 | 0.210 | 62.3 | 2.95 | 4/1 |
| Fused pyrrole | IND73602 | 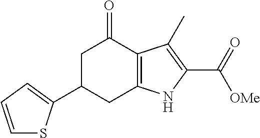 [SMILES: COC(=O)c1[nH]c2c(c1C)C(=O)CC(c1cccs1)C2] | 289.4 | 0.521* | 59.2 | 3.09 | 4/1 |
| | IND116088 | 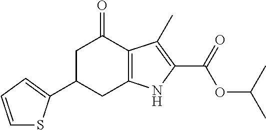 [SMILES: Cc1c(C(=O)OC(C)C)[nH]c2c1C(=O)CC(c1cccs1)C2] | 317.4 | 0.303* | 59.2 | 4.01 | 4/1 |
| | IND126429 | 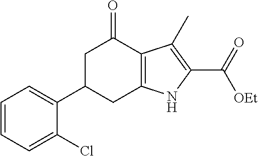 [SMILES: CCOC(=O)c1[nH]c2c(c1C)C(=O)CC(c1ccccc1Cl)C2] | 331.8 | 0.497* | 59.2 | 4.18 | 4/1 |
| | IND126432 | 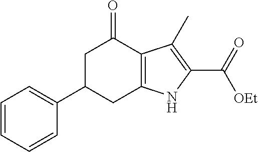 [SMILES: CCOC(=O)c1[nH]c2c(c1C)C(=O)CC(c1ccccc1)C2] | 297.4 | 0.144* | 59.2 | 3.56 | 4/1 |
| | IND87564 | 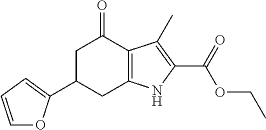 [SMILES: CCOC(=O)c1[nH]c2c(c1C)C(=O)CC(c1ccco1)C2] | 287.3 | 0.250 (1.125) | 83.96 | 2.80 | 4.5/1 |
| Piperazine | IND30802 | 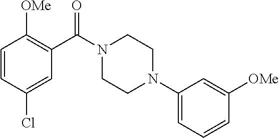 [SMILES: COc1cccc(N2CCN(C(=O)c3cc(Cl)ccc3OC)CC2)c1] | 365.3 | 0.124* | 32.8 | 4.17 | 4/0 |
| | IND116050 | 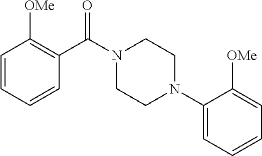 [SMILES: COc1ccccc1C(=O)N1CCN(c2ccccc2OC)CC1] | 326.4 | 0.887* | 42 | 2.89 | 5/0 |
| | IND126339 | 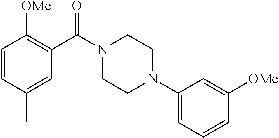 [SMILES: COc1cccc(N2CCN(C(=O)c3cc(C)ccc3OC)CC2)c1] | 340.4 | 0.248 | 42 | 3.17 | 5/0 |
| Sulfonamide | IND5418 | 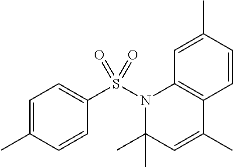 | 341.5 | 0.649* | 37.4 | 4.87 | 3/0 |
| | IND16365 | 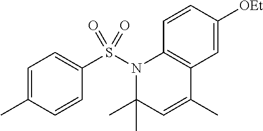 [SMILES: CCOc1ccc2c(c1)C(C)=CC(C)(C)N2S(=O)(=O)c1ccc(C)cc1] | 371.5 | 0.304 | 46.6 | 4.96 | 4/0 |
| | IND18762 | 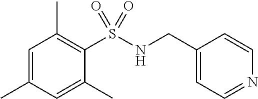 [SMILES: Cc1cc(C)c(S(=O)(=O)NCc2ccncc2)c(C)c1] | 290.4 | 4.128 | 59.1 | 2.11 | 4/1 |
| | IND86287 | 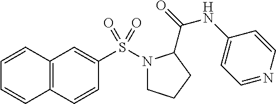 [SMILES: O=C(Nc1ccncc1)C1CCCN1S(=O)(=O)c1ccc2ccccc2c1] | 381.5 | 2.65* | 79.4 | 2.35 | 6/1 |
| | IND116071 | 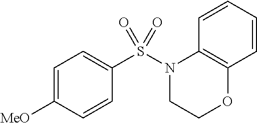 [SMILES: COc1ccc(S(=O)(=O)N2CCOc3ccccc32)cc1] | 305.4 | 1.71* | 55.8 | 2.15 | 5/0 |
| | IND126331 | 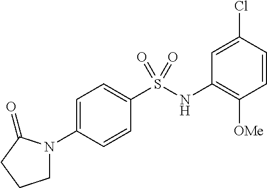 [SMILES: COc1ccc(Cl)cc1NS(=O)(=O)c1ccc(N2CCCC2=O)cc1] | 380.9 | 0.684* | 75.7 | 3.33 | 6/1 |
| Other | IND1323 | 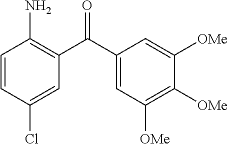 [SMILES: COc1cc(C(=O)c2cc(Cl)ccc2N)cc(OC)c1OC] | 321.8 | 0.085 | 70.8 | 3.21 | 5/1 |
| | IND5672 | 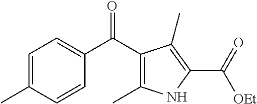 [SMILES: CCOC(=O)c1[nH]c(C)c(C(=O)c2ccc(C)cc2)c1C] | 285.3 | 0.593 | 59.2 | 3.68 | 3/1 |

^{a}Physicochemical parameters included MW = molecular weight; tPSA = total polar surface area relating to N and O atoms (TPSA_NO); xlogP = lipophilicity coefficient; HBA = H-bond acceptor and HBD = H-bond donor, and were calculated in Vortex v2011, Dotmatics Limited.
^{b}Mean ELISA EC₅₀, based on N = 3, except those noted (*), where N = 2. All EC₅₀ and cell viability values were >10 μM, all in T98G cells. Twenty-nine of these compounds were also tested in ELISA and cell viability assays in N2a-cl3 cells, with good activity seen in three, noted in (parentheses).

### Z. Scaffolds from PrP^{C} Assays

From SAR analysis of SPC hits in PrP^{C} assays using T98G cells, seven scaffolds representing amides, sulfonamides, AMT, indoles or fused indoles, chromenes, piperazines, and ureas were initially found. Six scaffolds-amide, sulfonamide, AMT, indole or fused indole, pyrimidine amide and chromene—were hits for both IMR32 and T98G cells. For the T98G cell line, active analogs were identified and confirmed from the amide, AMT, piperazine, chromene and sulfonamide scaffolds. In addition, several actives were found and confirmed from a fused pyrrole scaffold, which has some structural resemblance to the fused indole scaffold. Preliminary SAR trends become apparent for some of the scaffolds, such as the benzyl amide series where several analogs were purchased and tested along with the original SPC hit IND61769 (Table 26). The substitution required at the para-position of the benzene ring was probed; it was found that a bigger phenyl group (IND116065) is preferred. This compound is 10 times more potent than the bromo analog (IND61769). When replacing the bromine atom with a smaller chlorine atom, the compound (IND 126416) was found inactive at concentrations up to 10 μM. The methoxy analog (IND6612), which has a similar size to the bromo atom, has equal potency to the bromo analog in reducing PrP^{C} level in T98G cells.

TABLE 26
Preliminary SAR evaluating EC₅₀ values by ELISA
and calcein for selected amide analogs that lower
levels of PrP^{C} in T98G cells.

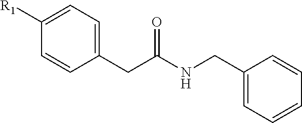

| | | EC₅₀ (μM) | |
|---|---|---|---|
| Compound | R₁ | ELISA | Calcein |
| IND61769 | 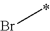 | 2.33 | >10 |
| IND6612 | 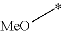 | 3.36 | >10 |
| IND126416 | 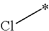 | >10 | >10 |
| IND116065 | 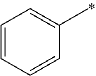 | 0.267 | >10 |

For IMR32 cells, in the follow up SAR-by-catalog efforts, none of the six potential scaffolds were confirmed; the confirmed SPC hits were found to be either inactive (EC₅₀>10 μM) or active at a concentration that also affected cell viability.

AA. Compounds that Lower Levels of PrP^{C} and PrP^{Sc}

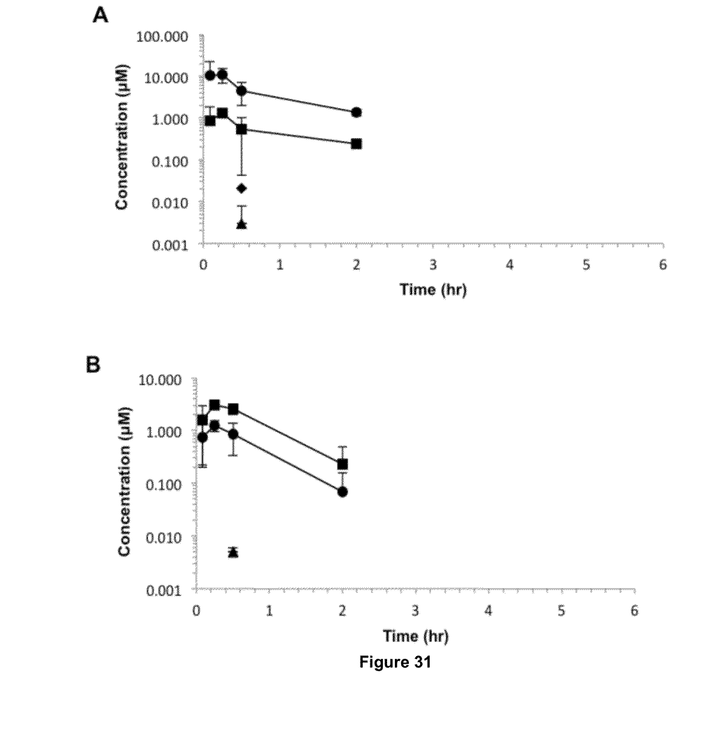

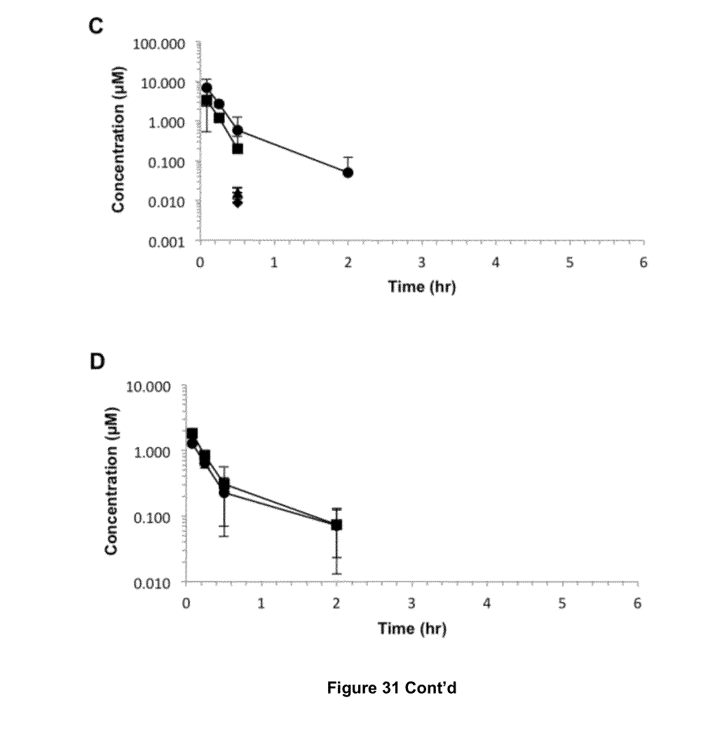

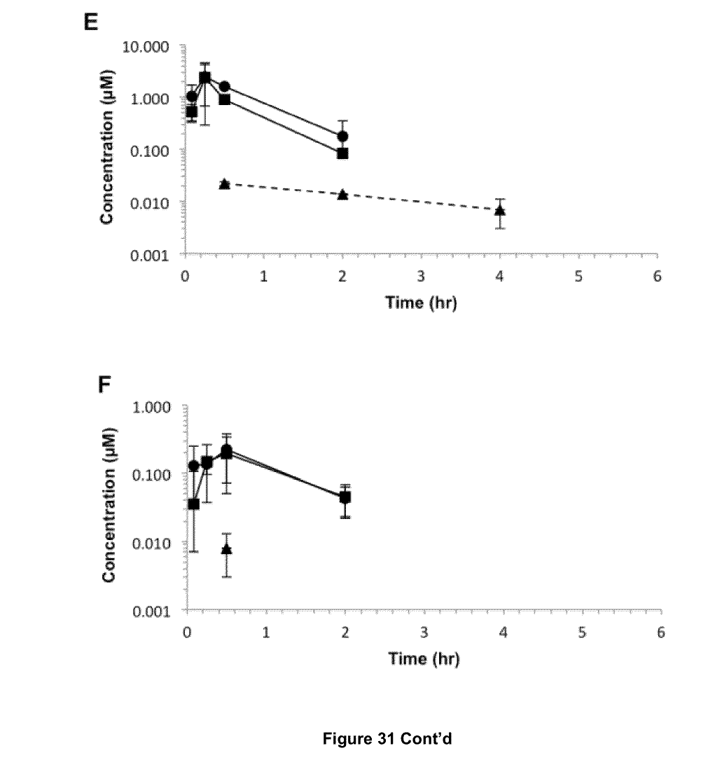

Because an ideal treatment for prion diseases would include drugs that lower levels of PrP^{C} and PrP^{Sc} (by decreasing its formation and/or increasing its clearance), it was determined if any compounds were identified as confirmed SPC hits in more than one of the four assays (PrP^{C} in T98G cells, PrP^{C} in IMR32 cells, PrP^{Sc} in dividing ScN2a-c13 cells, PrP^{Sc} in nondividing ScN2a-c13 cells), where PrP^{Sc} results were previously described (Silber et al., 2012b). There were 29 compounds that overlapped as confirmed SPC hits in the PrP^{C} assay in T98G and IMR32 cells, 36 compounds that overlapped as confirmed SPC hits in dividing and nondividing ScN2a-c13 cells, and one compound that overlapped as a confirmed SPC hit in the T98G and IMR32 PrP^{C} assays PrP^{Sc} assay in dividing cells; compound IND1270, a tetrahydroquinoline (FIG. 31).

BB. Stability

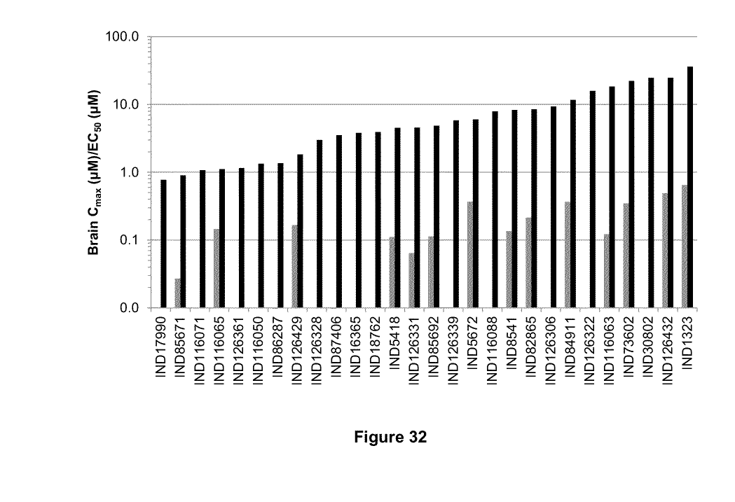

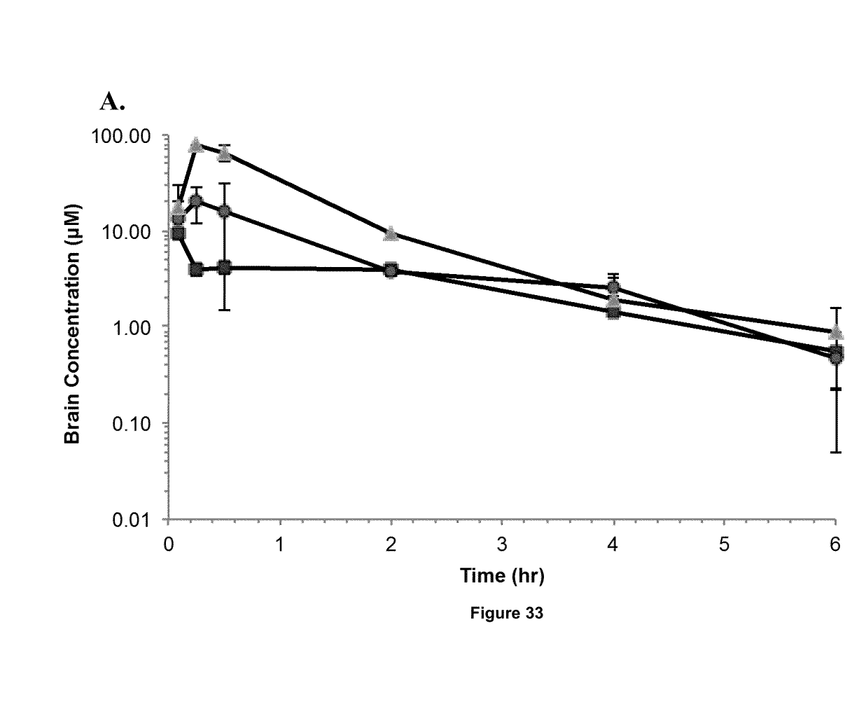

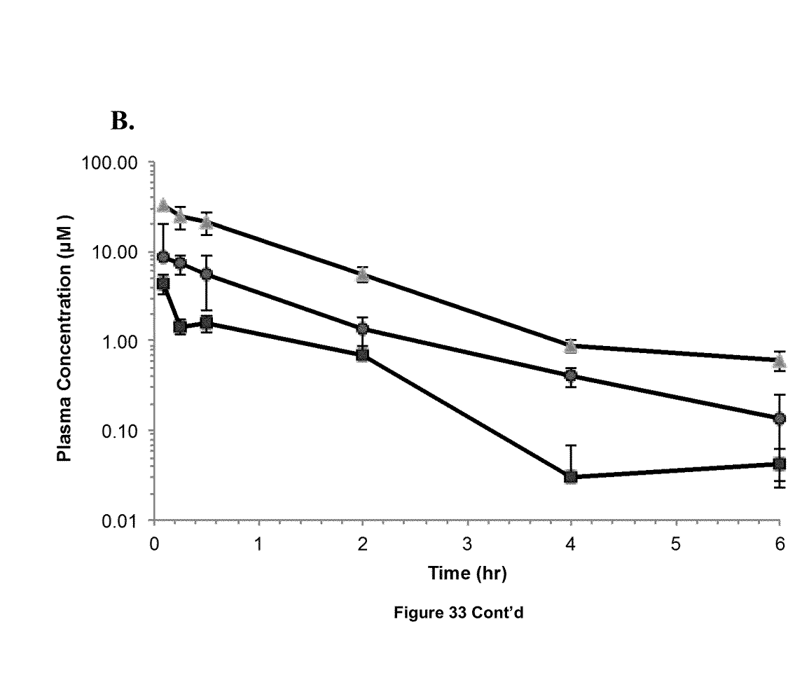

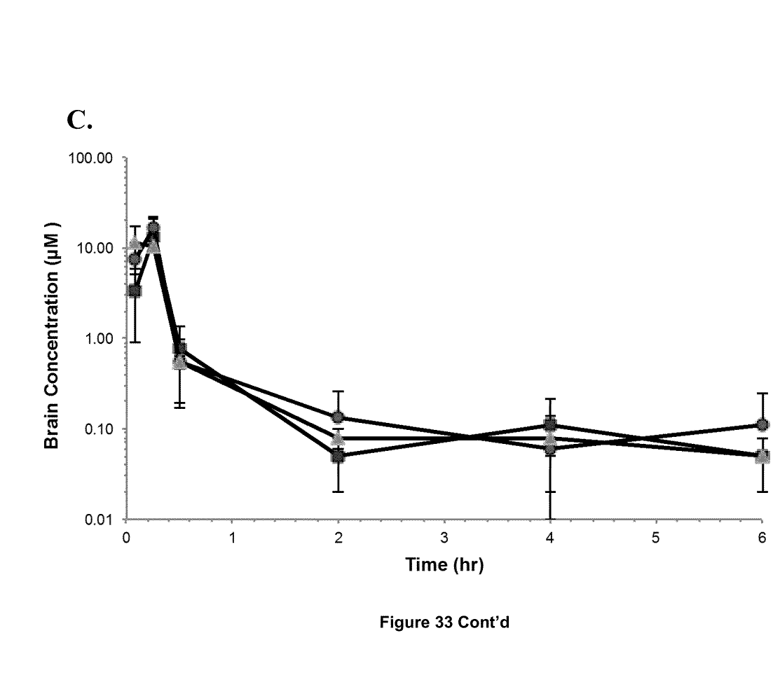

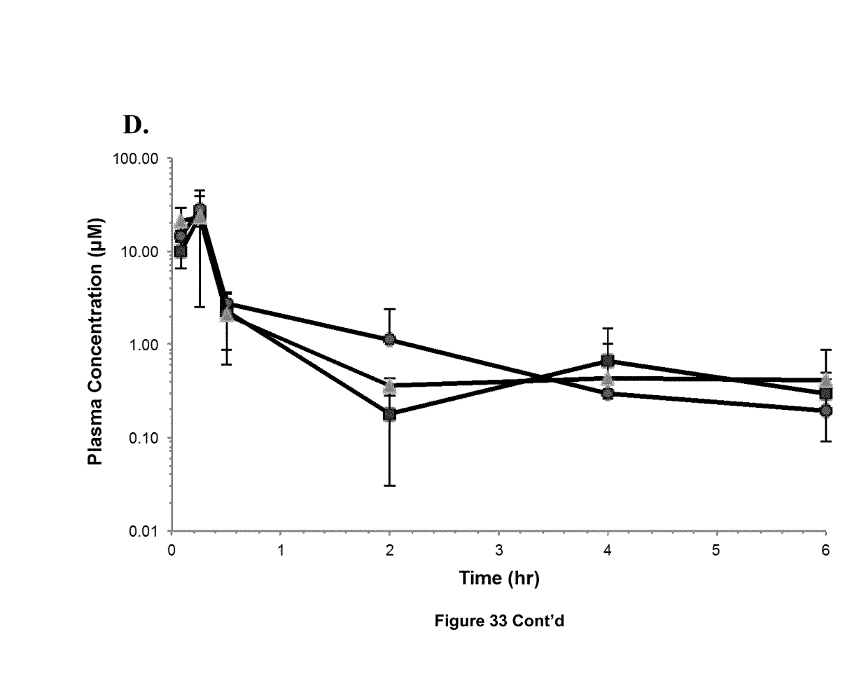

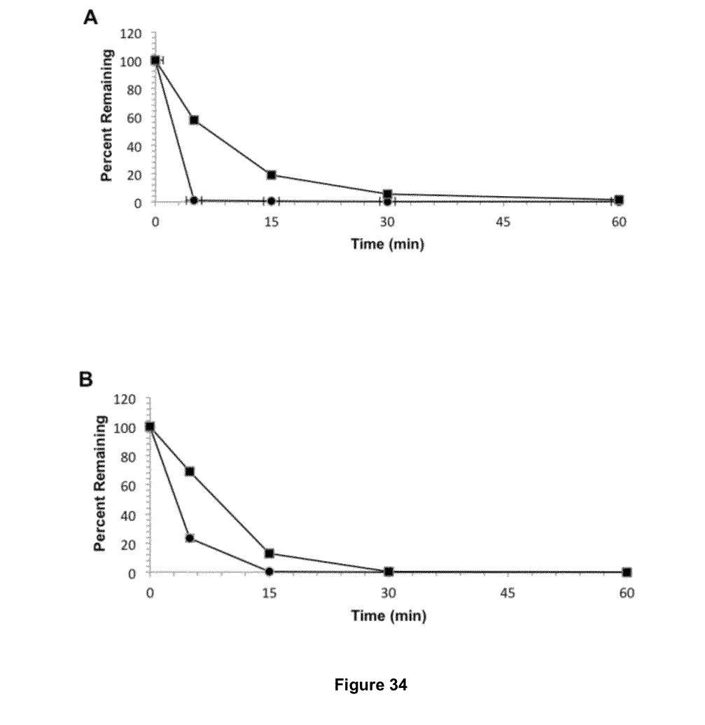

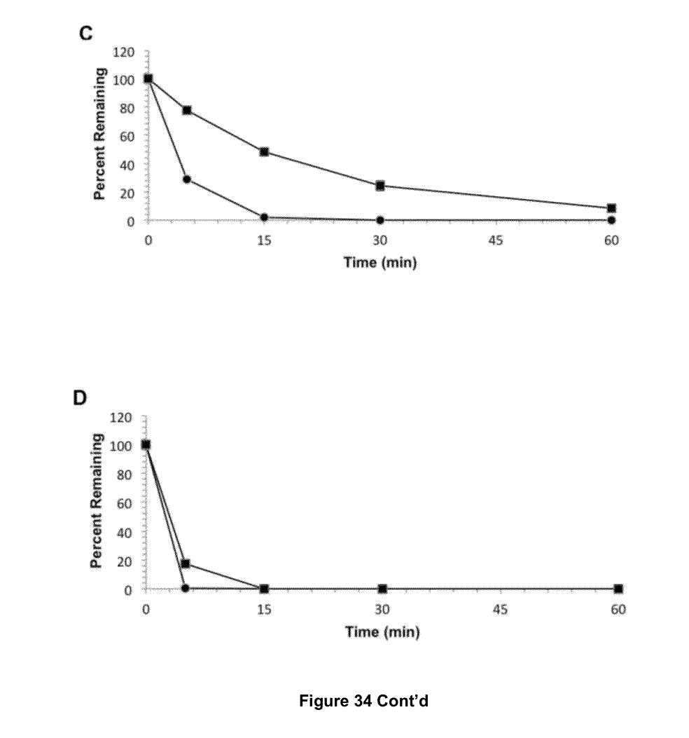

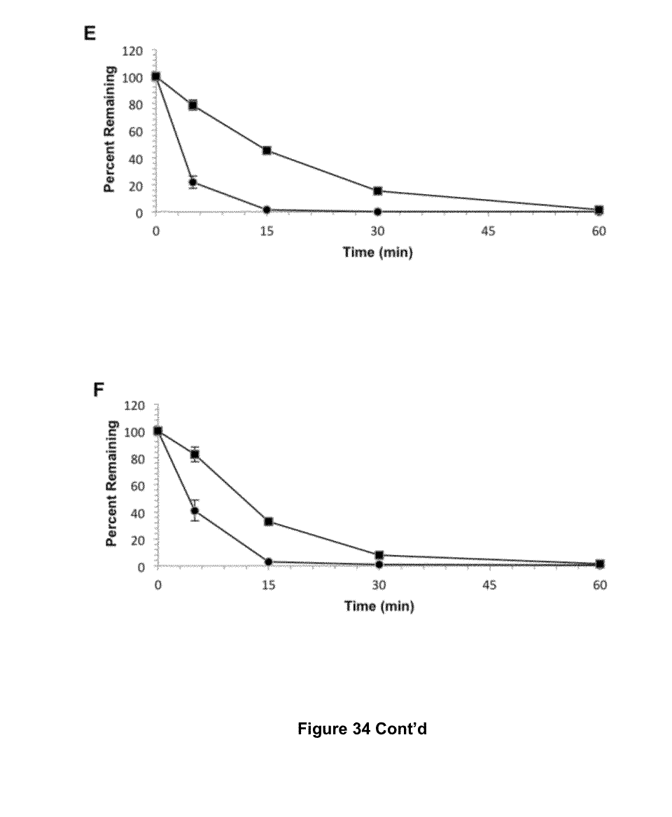

Twenty-eight of the 31 compounds that showed potency in ELISA (EC50=0.065 4.1 μM) and no effect on cell viability (EC50 >10 μM) in T98G cells were able to be purchased for pharmacokinetic testing in vivo. These were predicted to have good blood-brain barrier penetration properties. All 28 were dosed by oral gavage (PO) and by intraperitoneal (IP) injection at 10 mg/kg in female CD-1 mice, and then brain and plasma concentrations measured at various time points after dosing. Results showed that plasma and brain concentrations were barely or not measurable (<<0.1 μM) by liquid chromatography-mass spectrometry (LC/MS) after PO dosing, but were high (Cₘₐₓ up to 16.2 μM) after IP dosing. Brain and plasma concentration time curves are shown for six compounds representing six scaffolds are shown (FIG. 32). The ratio of the Cₘₐₓ in brain after single PO and IP dose to EC50 (Cₘₐₓ:EC50) is shown for all 28 (FIG. 33). Brain and plasma concentrations were also determined for two potent compounds after 20, 50, and 100 mg/kg/day once a day (QD) IP dosing to ensure that exposure could be maintained after repeated dosing. Both compounds were well tolerated and one resulted in high concentrations in the brain for at least six hours (10×EC50) (FIG. 34) at the doses studied.

Eight of 28 compounds were also tested for stability in mouse and human hepatic microsomes (Table 27) with decay curves shown for one representative compound from each of the six chemical scaffolds (FIG. 34). Results show that many are moderately or rapidly cleared in mouse and to a lesser extent in human microsomes.

TABLE 27
Stability of 8 of 31 confirmed SPC hits tested
in EC₅₀ assays, representing six scaffolds,
in mouse and human liver microsomes.

| | |
|---|---|
| t_{1/2} (min) | Hepatic Extraction Ratio |

| Compound | Mouse | Human | Mouse | Human |
|---|---|---|---|---|
| IND8541 | 1.71 | 20.3 | 0.97 | 0.77 |
| IND30802 | 6.23 | 13.0 | 0.91 | 0.84 |
| IND87406 | 6.35 | 37.1 | 0.91 | 0.65 |
| IND116065 | 12.3 | 11.1 | 0.83 | 0.86 |
| IND116071 | 1.53 | 4.53 | 0.98 | 0.94 |
| IND116088 | 6.88 | 23.9 | 0.90 | 0.74 |
| IND126328 | 9.64 | 21.9 | 0.86 | 0.76 |
| IND126339 | 3.20 | 10.3 | 0.95 | 0.87 |

CC. Characterization

From more than 1,000 HTS hits in T98G and IMR32 cells, we eventually tested more than 300 in both cell lines in potency assays. Seven and six scaffolds were identified in T98G and IMR32 cells, respectively, and 32 leads were ultimately confirmed with good to excellent potency and no effects on cell viability in T98G cells. These represented six chemical scaffolds. These were considered for further development as part of a lead identification and optimization process.

Twenty-eight of the 32 could be purchased and were tested in vivo in pharmacokinetic studies to evaluate their drug-like properties, especially their ability to achieve high concentrations in the brain. None yielded significant drug concentrations in plasma or brain after PO dosing. However, they resulted in very high concentrations after IP dosing, where the ratio of C_{maxIP}:C_{maxPO} for some compounds studied was 150. For many of these compounds, concentrations in brain even after a single IP dose of 10 mg/kg was higher than that targeted (10×EC₅₀) for proof-of-concept (POC) studies to determine which can drug lower levels of PrP^{C} in the brain. Preliminary results from the first two compounds studied by IP dosing QD for three days suggested that they were well tolerated and at least one of them may be able to achieve acceptable target concentrations in the brain with a QD or BID dosing regimen, especially at higher IP doses. Results from the microsomal incubation studies showed that the compounds were likely substrates for P450 mediated oxidative metabolism, where drug clearance was faster in mouse versus human microsomes, and where the rate of turnover varied significantly between compounds.

Confirmed SPC hits were identified that were overlapping in two and three assays, including the two PrP^{C} assays reported in this work and the PrP^{Sc} assay in dividing cells previously reported (Silber et al.). It is not surprising the PrP^{C} and PrP^{Sc} assays yield different confirmed SPC hits. This may be due to the fact that different cell lines and different assay conditions were used in each. Drugs may be found to lower levels of PrP^{C} and PrP^{Sc} as part of a cocktail of drugs to treat CJD.

DD. Scaffolds

52,830 diverse small molecules in dividing cells and 49,430 in nondividing cells were tested. This led to 3,100 hits and 970 single point confirmed (SPC) hits and 331 hits and 55 confirmed SPC hits in dividing and nondividing cells, respectively. Identified from HTS of 49,430 compounds in the nondividing cell assay were 331 HTS hits (0.65% hit rate). Fifty-five of 321 were confirmed as SPC hits in nondividing cells. Ten of 331 could not be re-tested because of inadequate supplies. Thirteen nondividing scaffolds were initially identified from the 331 hits, but only three were confirmed based on the 55 confirmed SPC hits, including 11 piperazines, six indoles and six ureas, with the remaining 32 being singletons or duplicates. Results for these 23 are summarized (Table 28). Five of the 55 confirmed SPC hits tested for potency (between 3.2 nM-10 μM) were shown to be inactive (EC₅₀>10 μM). The percent inhibition for these (at 10 μM) generally ranged from 30-50%. In the present work, using novel, reliable and robust HTS assays testing more than 50,000 diverse chemical compounds, we discovered 3,100 hits and 970 confirmed hits, (1.85% confirmed hit rate) in dividing cells. The confirmed hits generally derived from 14 distinct chemical scaffolds and produced 50 compounds (either from the original screen or related analogs) with submicromolar potency (PrP^{Sc} ELISA EC₅₀<1 μM) and little or no effect on cell viability (EC₅₀>10 μM). In contrast to the 3,100 HTS and 970 confirmed SPC hits in dividing cells, we discovered only 331 HTS and 55 confirmed SPC hits in nondividing cells (0.11% SPC hit rate). The 55 confirmed hits represented three chemical lead series. The 55 confirmed SPC hits included 23 analogs from the three chemical leads. Of these, one indole analog (IND23308) had a large effect in HTS (70% inhibition) and SPC assays (94% inhibition) and had low micromolar potency in both stationary-phase and dividing cells (EC50=7.5 and 1.58 μM, respectively) with no effect on cell viability. Twenty-three of the 55 were comprised of 11 piperazines, 6 indoles and 6 ureas, while 32 represented singletons or duplicates. Thus far, five of 55 tested in concentration-effect assays showed poor potency in the PrP^{Sc} ELISA (EC₅₀>10 μM), significant effects on cell viability, or both. An SAR-by-catalog approach has begun to test related analogs of the 23 to identify those with increased potency, in order to drive an SAR by synthesis program. It may also be necessary to screen another 50,000 or more compounds in HTS to identify more hits and leads.

TABLE 28
Twenty-three confirmed SPC hits in nondividing cells representing three scaffolds
(from a total of 55 confirmed SPC hits), many overlapping as confirmed SPC hits in dividing cells, along with
calculated physicochemical parameters; 32 other confirmed SPC hits in nondividing cells represented miscellaneous scaffolds.

| | | | | Inhibition of Baseline | | | | | | | | |
|---|---|---|---|---|---|---|---|---|---|---|---|---|
| | | | | PrP^{5c} (%) | | | | Calculated Physicochemical | | | | |
| | | | | | | Non- | | Parameters^{a} | | | | |
| Scaffold | | | | Dividing | | dividing | | | | | QP- | |
| (Murcko | Com- | | | Cells | | Cells | | | | HBD/ | Caco- | QPlog- |
| Fragment) | pound | Structure | MW | ELISA^{b} | V^{c} | ELISA | V | ClogP | PSA | HBA | 2 | BBB |
| Piperazine 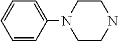 [SMILES: c1ccc(N2CCNCC2)cc1] | IND- 7190 | 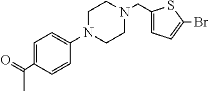 [SMILES: CC(=O)c1ccc(N2CCN(Cc3ccc(Br)s3)CC2)cc1] | 379.3 | 97 | 8 | 34 | 12 | 3.64 | 37.1 | 0/5 | 768.1 | 0.542 |
| | IND- 7486 | 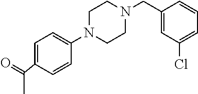 [SMILES: CC(=O)c1ccc(N2CCN(Cc3cccc(Cl)c3)CC2)cc1] | 328.8 | 84 | 6 | 31 | 12 | 3.67 | 36.8 | 0/5 | 785.8 | 0.435 |
| | IND- 17848 | 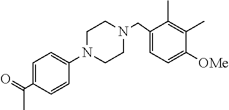 [SMILES: COc1ccc(CN2CCN(c3ccc(C(C)=O)cc3)CC2)c(C)c1C] | 352.5 | 95 | 1 | 41 | 24 | 3.81 | 42.3 | 0/5.8 | 767.9 | 0.167 |
| | IND- 18785 | 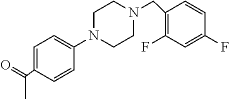 [SMILES: CC(=O)c1ccc(N2CCN(Cc3ccc(F)cc3F)CC2)cc1] | 330.4 | 85 | 30 | 30 | 20 | 3.56 | 36.8 | 0/5 | 785.8 | 0.475 |
| | IND- 19130 | 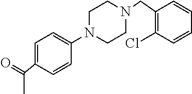 [SMILES: CC(=O)c1ccc(N2CCN(Cc3ccccc3Cl)CC2)cc1] | 328.8 | 86 | 5 | 41 | 2 | 3.54 | 37.0 | 0/5 | 776.9 | 0.393 |
| | IND- 18721 | 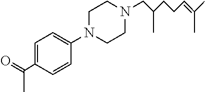 [SMILES: CC(=O)c1ccc(N2CCN(CC(C)CCC=C(C)C)CC2)cc1] | 328.5 | 78 | −6 | 55 | 10 | 4.24 | 36.2 | 0/5 | 765.6 | 0.033 |
| | IND- 19672 | 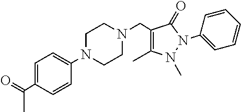 [SMILES: CC(=O)c1ccc(N2CCN(Cc3c(C)n(C)n(-c4ccccc4)c3=O)CC2)cc1] | 405 | 64 | −7 | 35 | −0.1 | 2.91 | 69.3 | 0/9 | 396.7 | −0.106 |
| | IND- 2118 | 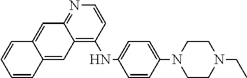 [SMILES: CCN1CCN(c2ccc(Nc3ccnc4cc5ccccc5cc34)cc2)CC1] | 382.5 | 76 | 25 | 51 | 24 | 5.19 | 29.6 | 1/4.5 | 1300.5 | 0.468 |
| | IND- 15461 | 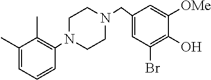 [SMILES: COc1cc(CN2CCN(c3cccc(C)c3C)CC2)cc(Br)c1O] | 405.3 | 22 | −4 | 48 | 3 | 4.46 | 34.1 | 1/4.5 | 1214.7 | 0.579 |
| | IND- 19847 | 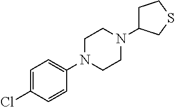 [SMILES: Clc1ccc(N2CCN(C3CCSC3)CC2)cc1] | 282.8 | 42 | −6 | 51 | 3 | 3.64 | 7.9 | 0/3.5 | 2396.0 | 1.253 |
| | IND- 7046 | 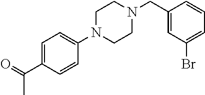 [SMILES: CC(=O)c1ccc(N2CCN(Cc3cccc(Br)c3)CC2)cc1] | 373.3 | 67 | 14 | 39 | 2 | 3.74 | 36.8 | 0/5 | 785.8 | 0.447 |
| Urea 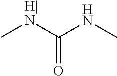 | IND- 5942 | 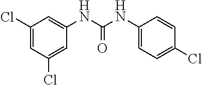 [SMILES: O=C(Nc1ccc(Cl)cc1)Nc1cc(Cl)cc(Cl)c1] | 315.6 | 91 | 3 | 51 | 8 | 3.90 | 46.7 | 2/2 | 1717.3 | 0.327 |
| | IND- 8255 | 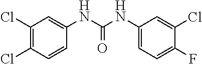 [SMILES: O=C(Nc1ccc(F)c(Cl)c1)Nc1ccc(Cl)c(Cl)c1] | 333.6 | 92 | 2 | 61 | 11 | 4.04 | 46.8 | 2/2 | 1720.5 | 0.388 |
| | IND- 8314 | 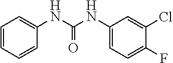 [SMILES: O=C(Nc1ccccc1)Nc1ccc(F)c(Cl)c1] | 264.7 | 87 | 7 | 56 | 17 | 3.11 | 46.7 | 2/2 | 1717.7 | 0.094 |
| | IND- 10851 | 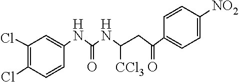 | 499.6 | 83 | 15 | 61 | 19 | 5.19 | 114.4 | 1/4 | 155.5 | −1.048 |
| | IND- 19237 | 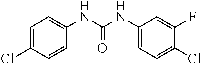 [SMILES: O=C(Nc1ccc(Cl)cc1)Nc1ccc(Cl)c(F)c1] | 299.1 | 25 | 2 | 66 | 26 | 3.60 | 46.7 | 2/2 | 171.6 | 0.256 |
| | IND- 19238 | 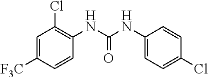 [SMILES: O=C(Nc1ccc(Cl)cc1)Nc1ccc(C(F)(F)F)cc1Cl] | 349.1 | 17 | −3 | 63 | 9 | 4.35 | 46.0 | 2/2 | 2005.2 | 0.485 |
| Indole 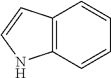 | IND- 5381 | 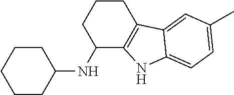 [SMILES: Cc1ccc2[nH]c3c(c2c1)CCCC3NC1CCCCC1] | 282.4 | 27 | 12 | 48 | 16 | 4.32 | 24.5 | 2/1.5 | 1527.6 | 0.630 |
| | IND- 15474 | 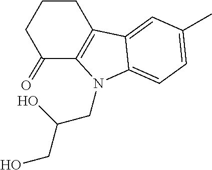 [SMILES: Cc1ccc2c(c1)c1c(n2CC(O)CO)C(=O)CCC1] | 273.3 | 14 | 11 | 39 | 9 | 2.01 | 70.6 | 2/5.4 | 776.4 | −0.795 |
| | IND- 10094 | 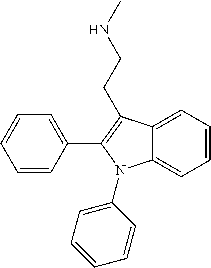 [SMILES: CNCCc1c(-c2ccccc2)n(-c2ccccc2)c2ccccc12] | 326.4 | 81 | 19 | 46 | 21 | 5.60 | 16.9 | 1/1.5 | 1471.9 | 0.551 |
| | IND- 4176 | 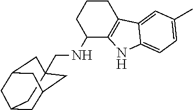 [SMILES: Cc1ccc2[nH]c3c(c2c1)CCCC3NCC12CC3CC(CC(C3)C1)C2] | 348.5 | 98 | 9 | 53 | −2 | 5.43 | 23.0 | 2/1.5 | 1696.6 | 0.610 |
| | IND- 23308 | 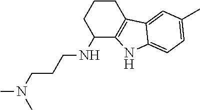 | 285.4 | 88 | 30 | 94^{d} | 12 | 3.45 | 31.7 | 2/3.5 | 289.4 | 0.684 |
| | IND- 22103 | 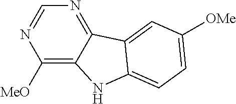 [SMILES: COc1ccc2[nH]c3c(OC)ncnc3c2c1] | 229.2 | 81 | −3 | 37 | −21 | 2.53 | 53.4 | 1/2.8 | 2945.9 | −0.017 |

^{a}Physicochemical parameters calculated using Qikprop (Schrödinger, New York, NY) {Barreiro, 2007 #9027; Dzierba, 2007 #9028}.
^{b}ELISA values from SPC assays: compounds reducing PrPSc levels by ≧30% are considered to have good antiprion potency.
^{c}V = cell viability percentages from SPC assays; compounds reducing cells ≦30% are considered to have a safe effect.
^{d}ELISA EC50 = 7.5 and 1.58 μM in stationary-phase and dividing cells, respectively, and for both, potency in cell viability was >10 μM.

### Overlapping Compounds Reducing PrP^{5c} in Dividing and Nondividing Cells

Three hundred thirty-one compounds were initially identified in HTS that reduced PrP^{Sc} in nondividing cells, but only 55 were subsequently confirmed as overlapping SPC hits in the 321 that could be re-tested. Thirty-six of the confirmed SPC hits in nondividing cells were also confirmed SPC hits in dividing cells (Table 28)

EE. Experimental Protocols

The methods employed to identify and confirm hits that lower levels of PrP^{Sc}, run concentration-effect (EC50) curves by ELISA and Western immunoblotting, and effects on cell viability in ScN2a-c13 cells are described below. Mouse N2a neuroblastoma cells (ATCC) were transfected with full-length mouse PrP and infected with the Rocky Mountain Laboratory strain of mouse-adapted scrapie prions, yielding dividing ScN2a-c13 cells (Ghaemmaghami et al., 2010b). ScN2a-c13 cells were seeded into black wall, clear bottom, tissue culture treated plates (Greiner) with 40000 cells/well (in 100 μL of assay medium: minimum essential medium (MEM) supplemented with 10% FBS, GlutaMax and 500 μg/mL Geneticin). Compounds were dissolved in 100% DMSO and diluted in assay medium at 20 μM final concentration (2× to achieve a test concentration of 10 μM) before addition to the assay plates (0.5% final DMSO concentration). Compound addition occurred 4 h after cell seeding into the assay plates. After 5 days incubation at 37° C. in a humidified and 5% CO2-enriched environment, lysates were generated as previously described and transferred to high binding ELISA plates (Greiner) coated with D18 primary antibody for overnight incubation at 4° C. The next day, the plates were washed three times with Tris-buffered saline Tween-20 (TBST) before addition of 100 μL of a 1:1000 dilution of HRP-conjugated D13 antibody in 1% BSA/PBS for a 1 h incubation at room temperature. After incubation with the D13 antibody, the plates were washed seven times with TBST, 100 μL of 2,2′-azino-bis(3-ethylbenzothiazoline-6-sulphonic acid (ABTS) was added to each well for 10 min, and absorbance at 405 nm was read using a SpectraMax M5 plate reader (Molecular Devices, Sunnyvale, Calif.). Calcein cell viability assays were run on separately seeded 96-well black wall plates. To evaluate cell viability (Ghaemmaghami et al., 2010b), mouse N2a-c13 cells were seeded into 96-well, black polystyrene plates (Greiner) and treated with compound as described above for the ELISA plates. After 5 days, the growth media was aspirated, the plates washed once with PBS (250 μL/well), and the plates aspirated dry. Calcein-AM (100 μL/well, 5 μg/mL solution in calcium- and magnesium-free PBS) was added, and the plates were incubated at 37° C. for 45 min. Fluorescent emission intensity was quantified using a Spectramax M5 plate reader, excitation/emission spectra of 485 nm/530 nm. To perform Western immunoblotting, ScN2a-c13 cells, cultured as described above, were seeded onto 6-well, tissue culturetreated dishes at a density of 1.54×10⁶ cells/well in 6.2 mL of supplemented MEM and allowed to adhere for 4 h at 37° C. Test compounds diluted in supplemented MEM (described above) were added to the plate (0.8 mL/well) to attain final concentrations ranging from 1 nM to 32 μM. After 5 days, the media was aspirated from each well and the plates washed one time with PBS (7 mL/well). The cells were lysed by addition of 0.35 mL of lysis buffer (20 mM Tris HCl, pH 8.0; 100 mM NaCl; 0.5% NP-40; 0.5% sodium deoxycholate; and 7.5 U/mL benzonase). The total protein in the lysate was measured using a bicinchoninic protein assay (Pierce). A total of 0.06 mg protein was treated with proteinase K (PK) (total protein:enzyme ratio=50:1) in 0.1 mL PBS and the sample incubated at 37° C. for 1 h. The proteolytic digestion was terminated by the addition of PMSF to a final concentration of 3 mM. The samples were centrifuged at 16,000×g for 1 h, the supernatant discarded, and the pellets resuspended in 15 μL of reducing SDS sample buffer. The PrP^{Sc}-containing samples were denatured by heating at 80° C. for 5 min and run in a 4-12% Tris glycine SDS gel (Invitrogen). The gels were transferred to nitrocellulose membrane using a Nupage apparatus (Invitrogen) and the membranes blocked with 5% (w/v) nonfat milk/TBST overnight. The membranes were immersed in a 1:10000 dilution of D13-HRP antibody (1 mg/mL) for 1 h at RT, washed 3 times with TBST buffer before development (1 min) with Enhanced Chemiluminescent Western Blot reagent (GE Healthcare). Imaging and quantification of the blots were done using a Gene Gnome (Syngene) equipped with Gene Tools software.

FF. Compound Characterization

Another set of experiments was conducted to include the positive control (Compd B), and test IND24 and IND81 in mouse, rat, dog (male and female) and human (mixed) hepatic microsomes to determine if there were gender effects. Stock solutions of 0.5 mM IND24, IND81, and Compd B were prepared in DMSO. These were diluted 500-fold into 1 mL of microsomal incubation mixture to yield a final concentration of 1 μM. The incubation mixture was composed of 100 mM phosphate buffer, pH 7.4, and NADPH regenerating system (BD Biosciences NADPH Regenerating System Solutions A & B). This mixture was preincubated at 37° C. for 5 min in an Eppendorf Thermo mixer, and the reaction initiated by addition of 0.5 mg (25 μl of a 20 mg/mL solution) of liver microsomes. Aliquots (100 μl) were withdrawn at 0, 5, 15, 30, and 60 min, and added to 100 μA ACN containing internal standard. After centrifugation at 12,000×g for 10 min, the supernatants were analyzed by LC/MS/MS. The percentage of solute remaining at the end of the incubation was used to determine species-specific values of scaled intrinsic clearance, hepatic clearance, and predicted hepatic extraction based upon the calculations below (Obach, 1999).

### Intrinsic Clearance:

$${Cl}_{int , microsomal} = \frac{0.693}{In vitro {T}_{1 \text{/} 2}} * \frac{ml incubation}{mg microsomes}$$

### Scaled Intrinsic Clearance:

$${Cl}_{int , scaled} = \frac{0.693}{In vitro {T}_{1 \text{/} 2}} * \frac{ml incubation}{mg microsomes} * \frac{mg microsomes}{gm liver} * \frac{gm liver}{kg body weight}$$

for which mg microsomal protein/gm liver was 45 for all species, and gm liver/kg body weight were 32 for dog, 88 for mouse, 40 for rat and 25 for human.

Hepatic Clearance:

Clₕ=(Q*Cl_{int,scaled})/(Q+Cl_{int,scaled})

for which Q was hepatic blood flow; values of 90 ml/min/kg for mouse, 66 ml/min/kg for rat, and 21 ml/min/kg for human were used.

### Hepatic Extraction Ratio (ER):

ER=Clₕ/Q

for which ER estimates the amount of compound cleared during a single pass through the liver (Low ER <0.3, moderate ER 0.3-0.7, high ER>0.7).

GG. Single Dose Pharmacokinetic Studies

Two hundred thirty-five 2-AMTs were synthesized and tested for antiprion potency in dose-titration EC₅₀ studies using an ELISA-based assay (Silber et al.). Of these, 34 2-AMT compounds were selected for testing in single-dose oral pharmacokinetic (PK) studies, based on potency, structural diversity and physicochemical parameters, including solubility, in some cases, stability in (mouse, rat, dog, and human) liver microsomes, and permeability. Twenty-seven compounds were evaluated at a single oral dose of 40 mg/kg; seven 2-AMTs were administered at 10 mg/kg. Six 2-AMTs were evaluated at both doses. The lower dose was chosen in the second round to provide a linear range.

Pharmacokinetic protocols employing mice were reviewed and approved by the UCSF Institutional Animal Care and Use Committee (IACUC). Female FVB mice, weighing approximately 25 g, were used for all in vivo pharmacokinetic studies. Mice were housed with free access to food and water, and were maintained on 12-h light/dark cycles for 1 week before dosing studies were initiated.

For the single-dose of 40 mg/kg, compounds were dissolved in a formulation containing 5% propylene glycol, 35% α-tocopheryl polyethylene glycol 1000 succinate (TPGS) and 70% polyethylene glycol 400 (PEG400), and administered by oral gavage. Two animals per time point were used. At specified time points after dosing (0.5, 1, 2, 4, 6, and 24 h), animals were euthanized by CO₂, and blood for plasma (by cardiac puncture) and brain samples were collected. The heparinized blood samples were centrifuged to obtain plasma. Brain samples were weighed, diluted 10-fold with water, and then homogenized using a Precellys 24 tissue homogenizer. The brain and plasma samples were flash-frozen on dry ice and then stored at −80° C. until analysis. For the single-dose administration at 10 mg/kg, compounds were dissolved in a formulation containing 20% propylene glycol, 5% ethanol, 5% labrosol, and 70% PEG400, and administered by oral gavage. Two animals per time point were used. At specified time points after dosing (0.25, 0.5, 1, 2, 4, 6, and 24 h), animals were euthanized by CO₂, and blood for plasma (by cardiac puncture) and brain samples were collected. The heparinized blood samples were centrifuged to obtain plasma. Brain samples were weighed, diluted five-fold with water, and then homogenized using a Precellys 24 tissue homogenizer. The brain and plasma samples were flash-frozen on dry ice and then stored at −80° C. until analysis.

IND24, IND81, IND22 and Compd B were evaluated by single-dose pharmacokinetic studies administered at 1 mg/kg by intravenous (IV) injection. IND81 and Compd B were dissolved in either 10% DMSO in PEG 400/water (1:1) or 10% DMSO; IND24 and IND22 were dissolved in 10% ethanol in PEG 400/water (1:1). Compounds were administered through tail vein injection. Two animals per time point were used. At specified time points after dosing (5 min, 0.25, 0.5, 1, 2, 4, and 6 h), animals were euthanized by CO₂. Blood and brain samples were obtained and processed as described above for the oral dosing at 40 mg/kg.

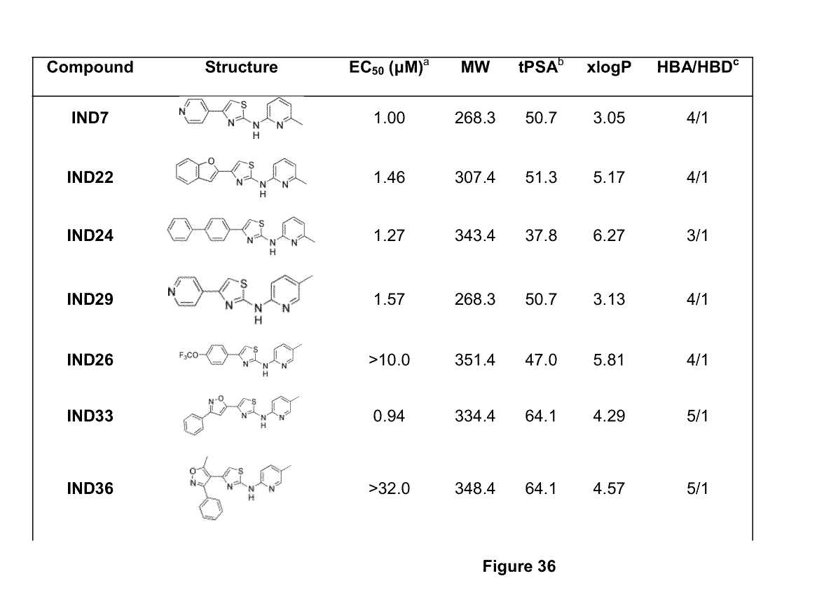

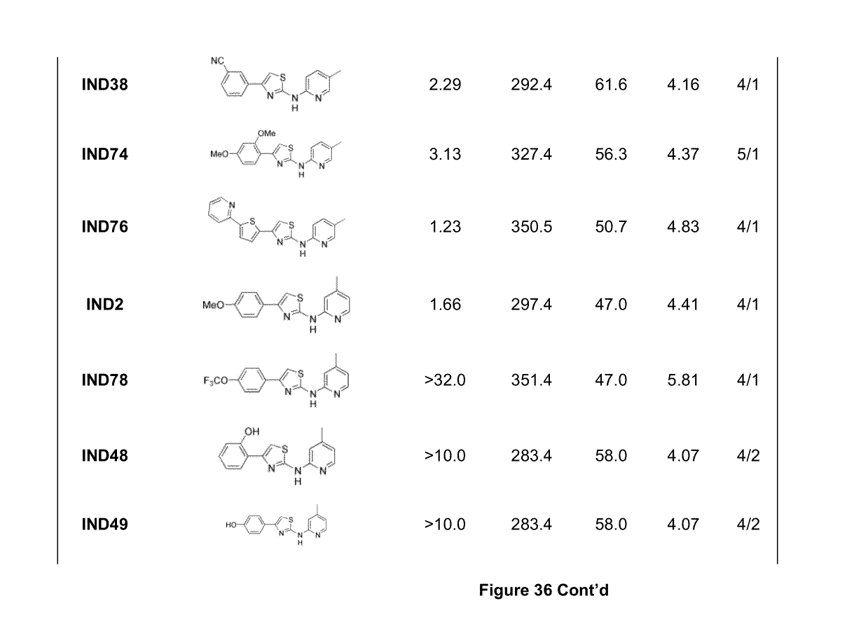

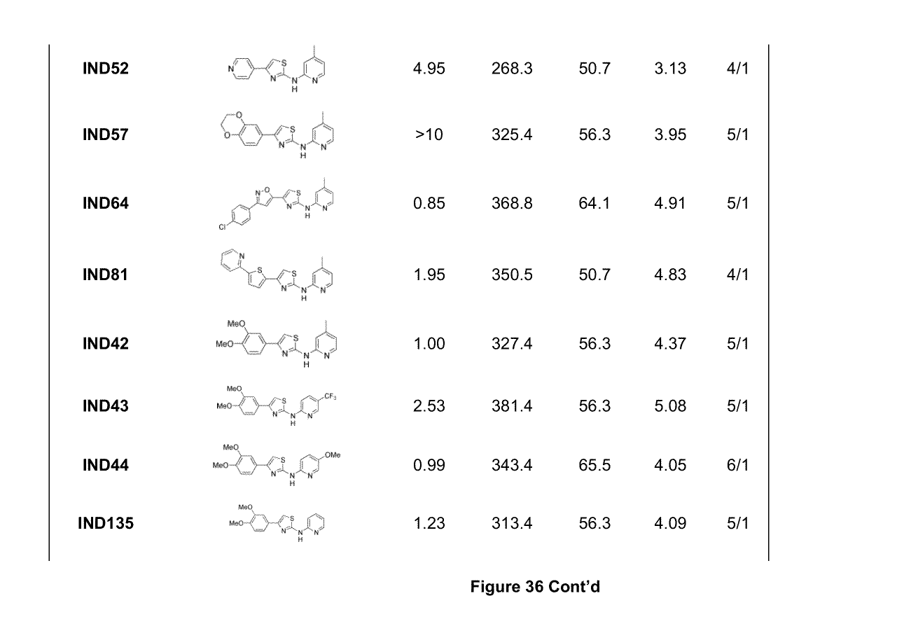

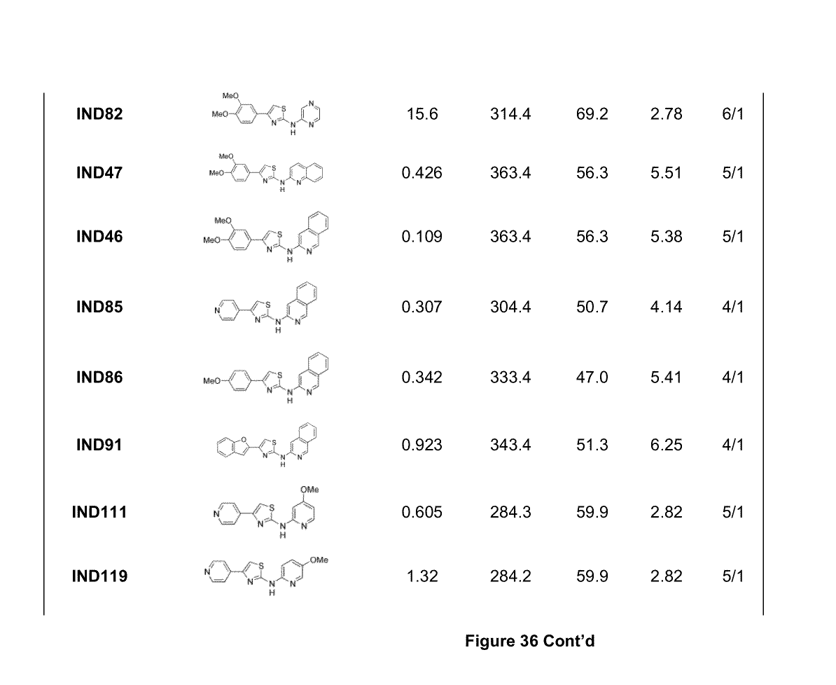

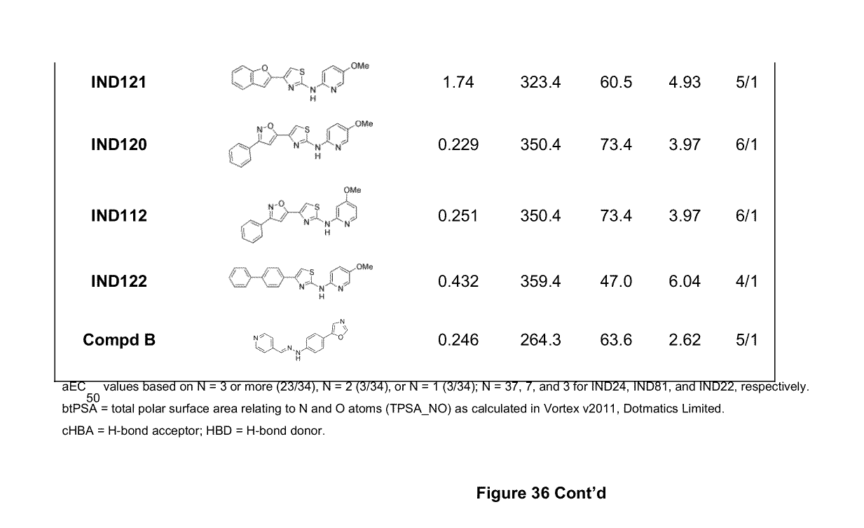

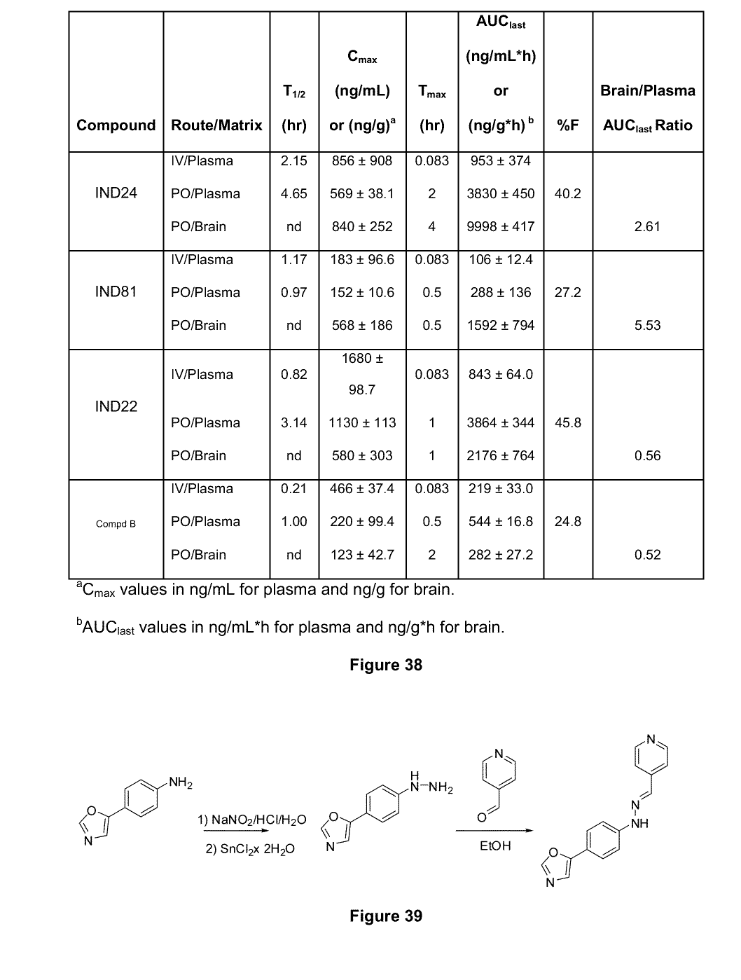

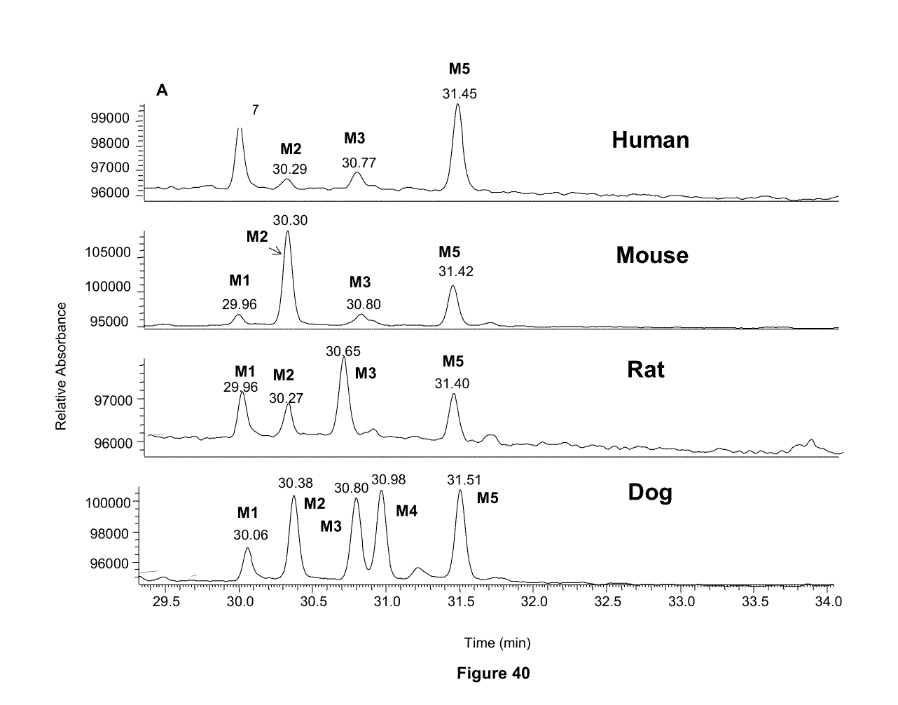

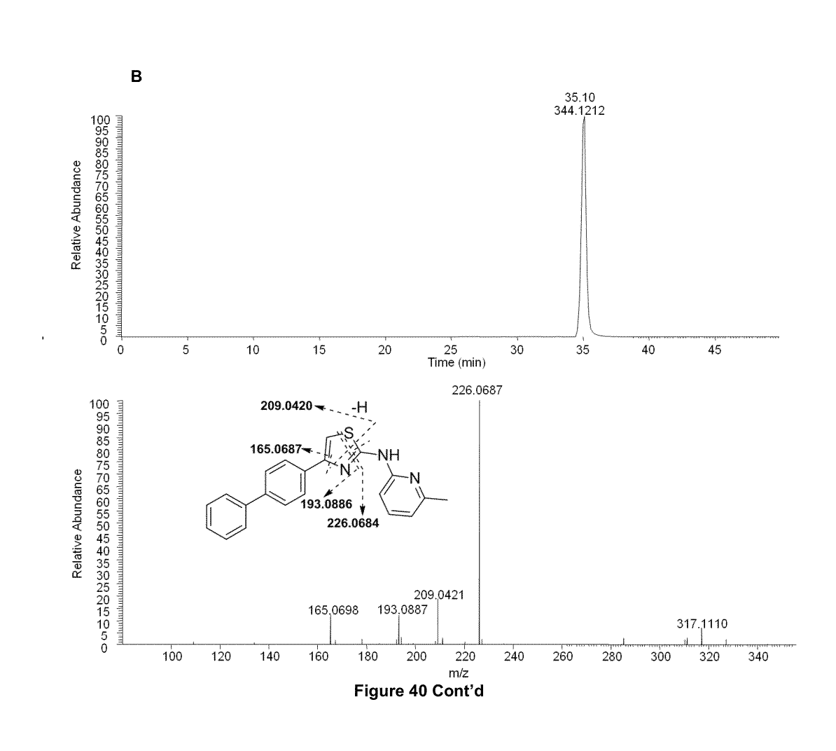

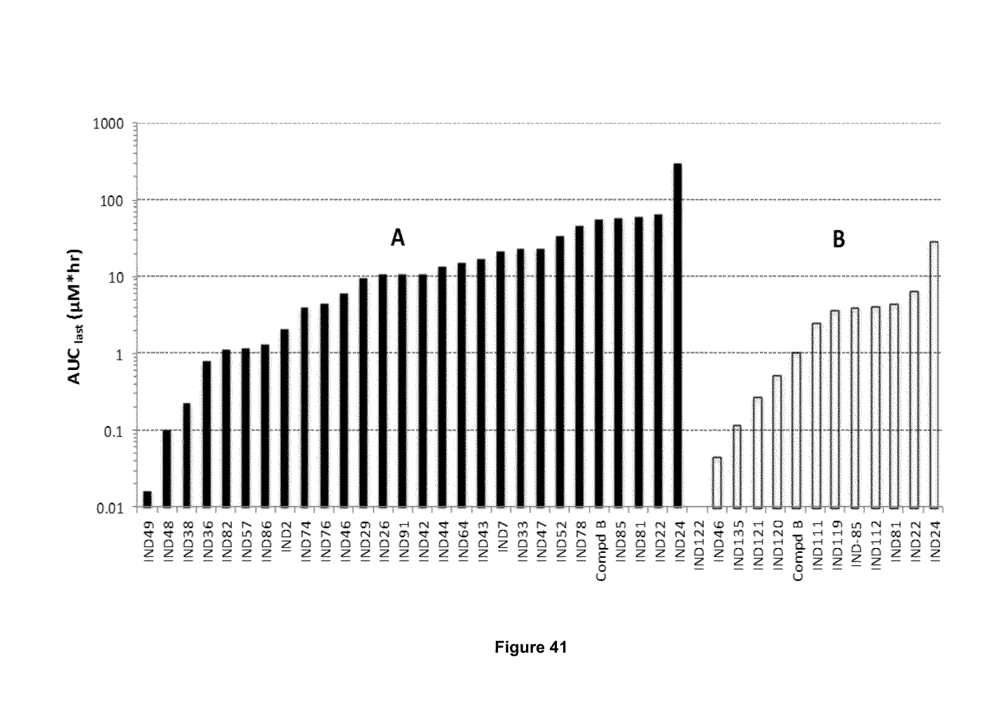

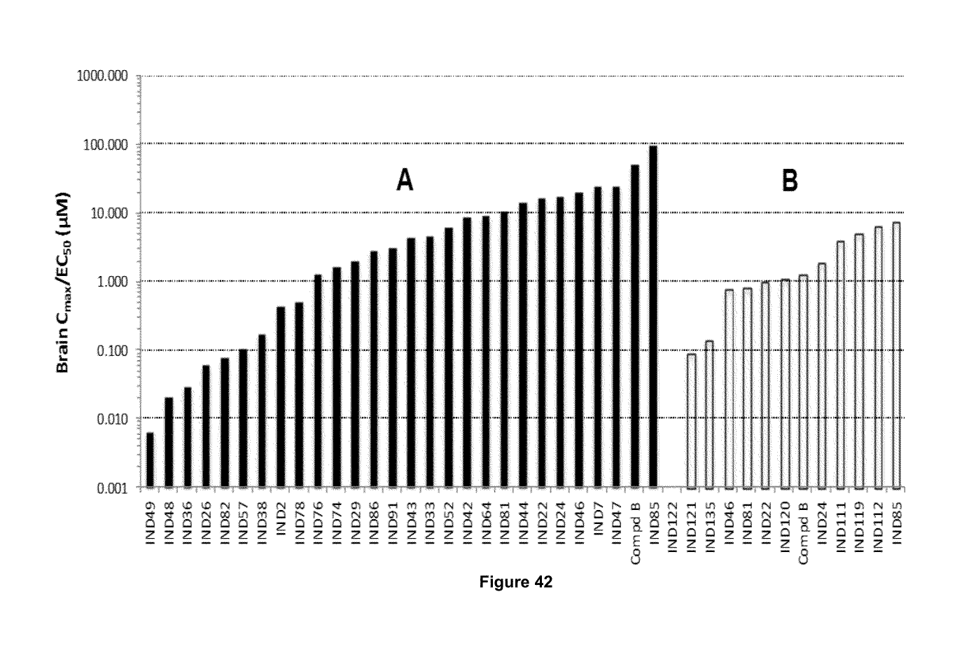

To determine the pharmacokinetics of the 2-AMTs, we administered all 34 2-AMTs and Compd B in single oral doses to FVB mice. Doses were 40 mg/kg for 28 compounds and 10 mg/kg for 13 compounds; IND22, IND24, IND46, IND81, IND85, and Compd B were administered at both dosages (FIG. 41 and FIG. 42). Brain exposure (AUCᵢₐₛₜ values) spanned four orders of magnitude across the 27 2-AMT analogs and Compd B examined at 40 mg/kg (FIG. 41A). IND48 and IND49 had the lowest AUCₗₐₛₜ values; this observation is not surprising because they were the only two compounds examined that had more than one hydrogen bond donor. The range of AUCₗₐₛₜ values was less broad for the 12 analogs at 10 mg/kg doses (FIG. 41B). Both IND24 and IND81 showed very good brain exposures, with brain:plasma AUCₗₐₛₜ ratios of 2.6 and 5.5, respectively, compared to a ratio of 0.5 for both IND22 and Compd B (FIG. 38). Comparison of the calculated PSA values (FIG. 36) to the observed AUCₗₐₛₜ values (FIG. 41) showed only a modest correlation. We found no apparent correlation between molecular weight and brain exposure.

At both doses studied, the majority of 2-AMT analogs had maximal brain concentrations (Cₘₐₓ values) that exceeded their in-vitro EC₅₀ values (ratios >1; FIG. 42). For 8 analogs at the 40 mg/kg dose, including IND24 and IND81, the Cₘₐₓ/EC₅₀ ratio exceeded 10 (FIG. 42A).

In addition to the oral doses, IND24, IND81, IND22 and Compd B were administered at 1 mg/kg to female FVB mice by the IV route. The half-life of IND24 was 2 h, which was 2× and 10× longer than that of IND81 and Compd B, respectively (FIG. 38). IND24 also had higher oral bioavailability (40%) compared to IND81 and Compd B (25-27%), while that for IND22 was slightly higher (46%).

### Multidose Pharmacokinetics

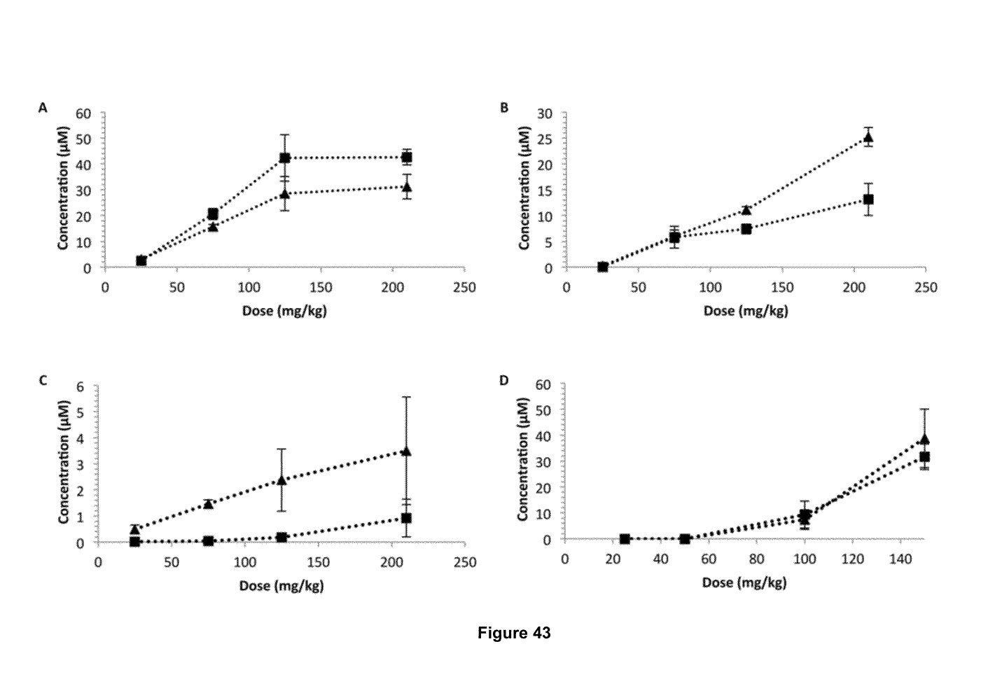

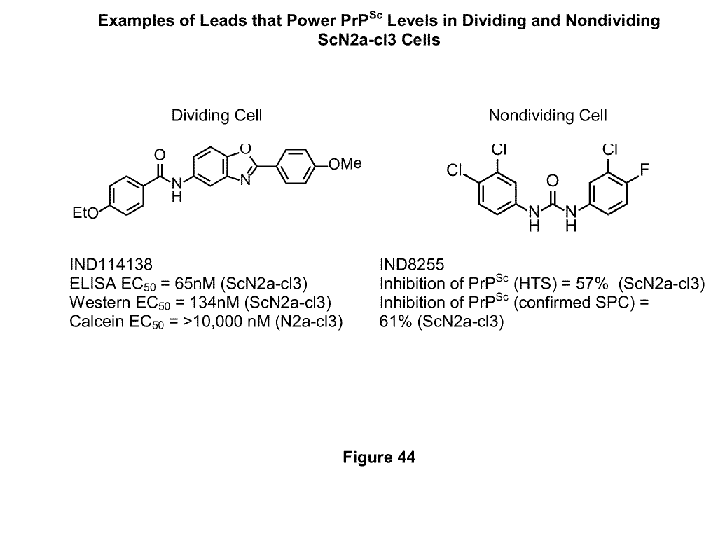

From the single-dose studies, 10 2-AMT analogs were ultimately selected for multidose pharmacokinetic studies. IND24, IND81 and IND22 were first administered at 25, 50, 125, and 210 mg/kg/day, while Compd B was dosed at 25, 50, 100, and 150 mg/kg/day to FVB mice for 3 days in a liquid diet, then brain and plasma concentrations measured (FIG. 43). The 10 AMT compounds were also dosed at 40, 80, 130 and 210 mg/kg/day for 3 days as above. IND24, IND81 and IND33 achieved the highest concentrations in both brain and plasma for doses >40 mg/kg. For IND24, both brain and plasma concentrations appeared to reach a plateau at 125-130 mg/kg, with not much increase when the dose was raised to 210 mg/kg. IND81 showed linear increases in brain and plasma concentrations with increasing doses. IND33 showed a dose-dependent increase in plasma, but peaked at 30 μM with the 130 mg/kg dose in brain. IND52 had no measurable brain concentrations at a dose of 40 mg/kg, while exposure was comparable at all other doses. IND22 showed comparable brain and plasma concentrations at all doses. The brain and plasma concentrations of 5 analogs (IND85, IND44, IND46, IND47, IND91) were too low (≦1 μM) to be of therapeutic value. A toleration study, where mice were dosed with IND24, IND81, and IND22 at 210 mg/kg/day for 14 days, indicated that these compounds were well tolerated.

Additionally, the pharmacokinetics of Compd B was evaluated to determine the suitable dose for use as a positive control in future in-vivo drug efficacy studies. Doses of 25, 50, 100, and 150 mg/kg were administered to FVB mice for 3 days in a liquid rodent diet, and then brain and plasma concentrations measured (FIG. 43D). All doses were tolerated, with no animals showing lethal toxicity. Doses of ≧150 mg/kg/day for 8 days resulted in 2/4 animals dying (data not shown). A dose of 100 mg/kg/day resulted in brain concentrations ˜10 μM (FIG. 43D), which is 25× the EC₅₀ value of 0.4 μM in ScN2a-c13 cells (data not shown).

HH. Compound Characterization

For LC/MS quantification for all 2-AMTs, samples and their respective internal standards were injected into either a BetaBasic C18 or BDS Hypersil C8 column. The solvent system used for separation was composed of water and ACN containing 1% formic acid. For quantification of IND24, IND22, and IND81, samples (along with a proprietary internal standard) were injected onto a BetaBasic C18 column maintained at room temperature. The amount of ACN in the gradient was increased from 75% ACN to 95% ACN over 2.5 min, held for 0.5 min, and then re-equilibrated to 75% ACN over 1.4 min. Data acquisition used MRM in the positive ion mode, and the transitions monitored were m/z 344→226 for IND24; m/z 351→233 for IND81; and m/z 363→245 for internal standard.

For quantification of Compd B, samples (along with a proprietary internal standard) were injected onto a BDS Hypersil C8 column maintained at room temperature. The amount of ACN in the gradient was increased from 25% ACN to 95% ACN over 2.0 min, held for 1.0 min, and then re-equilibrated to 25% ACN over 1.4 min. Data acquisition used MRM in the positive ion mode, and the transitions monitored were m/z 265→160 for Compd B and m/z 321→253 for internal standard.

For quantification of warfarin, samples (along with chlorowarfarin as internal standard) were injected onto a BetaBasic C18 column maintained at room temperature. The amount of ACN in the gradient was increased from 70% ACN to 95% ACN over 1.8 min, held for 0.5 min, and then re-equilibrated to 70% ACN over 1.4 min. Data acquisition used MRM in the negative ion mode, and the transitions monitored were m/z 307→161 for warfarin and m/z 341→161 for chlorowarfarin.

For quantification of dextromethorphan, samples (along with d3-dextromethorphan as internal standard) were injected onto a BDS Hypersil C8 column maintained at room temperature. The amount of ACN in the gradient was increased from 50% ACN to 95% ACN over 2.5 min, held for 1.5 min, and then re-equilibrated to 50% ACN over 1.0 min. Data acquisition used MRM in the positive ion mode, and the transitions monitored were m/z 272→215 for dextromethorphan and m/z 275→215 for d3-dextropethorphan.

II. Antiprion Potency and Calculated Physicochemical Parameters

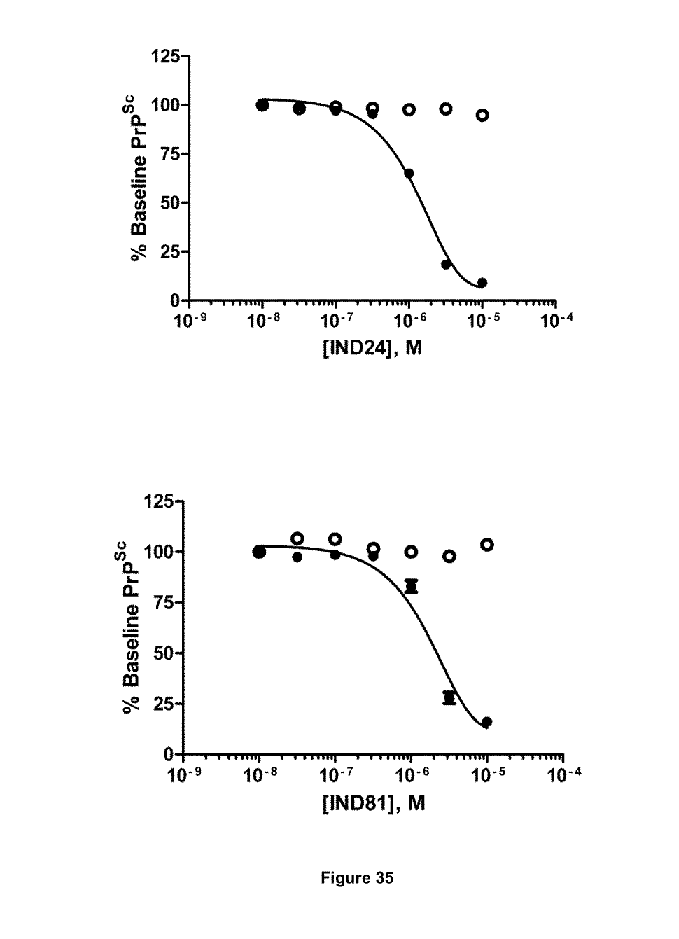

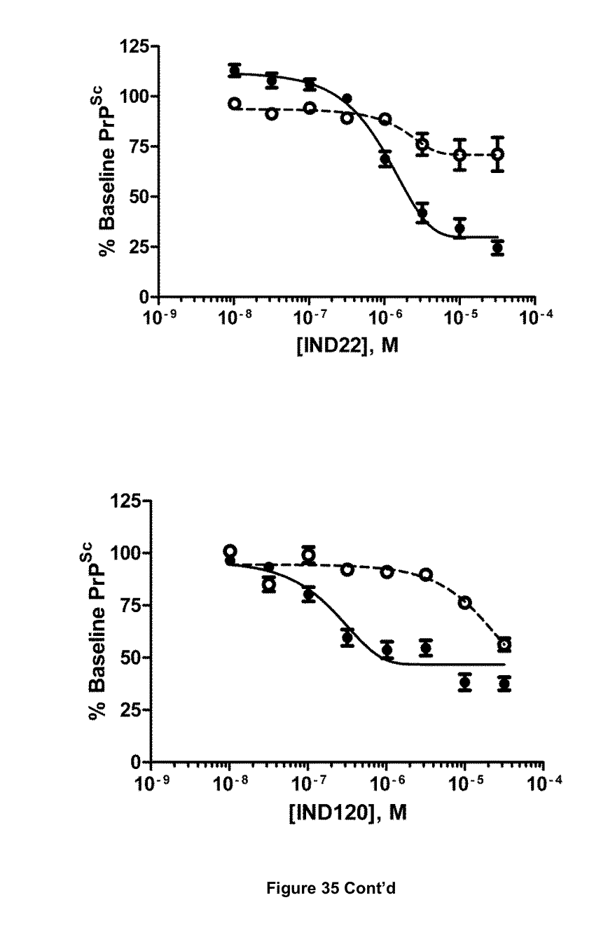

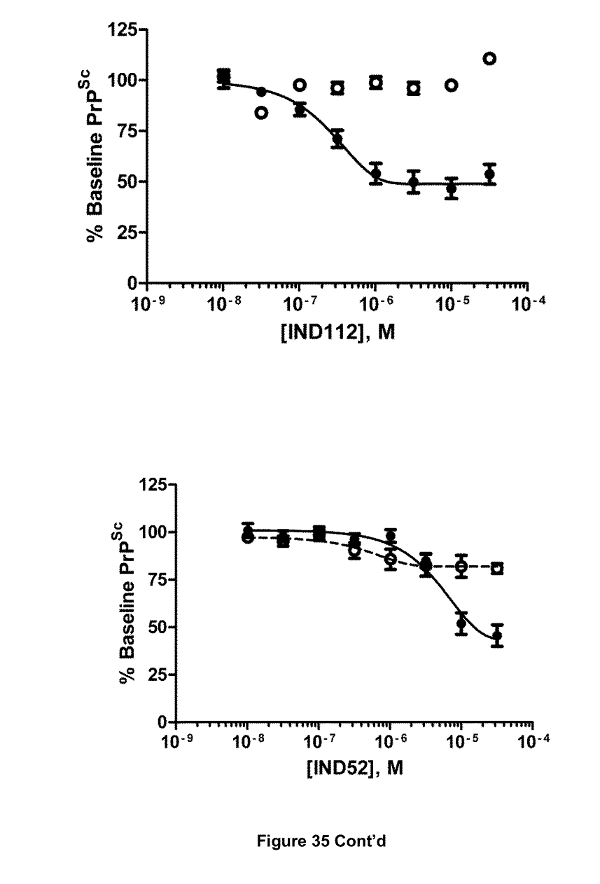

Potency was defined as the EC₅₀ value, calculated as the concentration at which there was a 50% reduction in PrP^{Sc} levels from control (DMSO treated cells). Potent effects were observed in the absence of effects on cell viability (EC₅₀>10 μM). FIG. 35 depicts EC₅₀ curves for six 2-AMTs, including the three leads. Concentration-effect relationships (EC₅₀) were obtained using eight concentrations per curve ranging from 10 nM to 32 μM, escalating by half-log increments. From the EC₅₀ values and physicochemical parameters for the 34 2-AMT compounds and Compd B (FIG. 36), we selected 10 analogs for further characterization in vitro. We previously showed that EC₅₀ values by ELISA were strongly correlated with those by Western immunoblotting, where R²=0.75; p<0.001 (Silber et al., submitted for publication). Calculated parameters included total polar surface area (tPSA), xlogP, and number of hydrogen bond donors and acceptors. The calculated values, such as xlogP and PSA, were generally within the range of acceptable values for CNS drugs.

JJ. Compound Characterization

A subset of the 235 analogs was evaluated to select at least two compounds for preclinical testing. Thus far, except for Compd B, no drug has been able to extend survival in these models beyond ˜70-150 days, depending on the model. From the first ˜100 analogs made, 34 were evaluated in single-dose pharmacokinetic studies to determine if any would be good preclinical development candidates (FIG. 36). Selection was based on assessing drug-like properties, which included low EC₅₀ values (FIG. 36), good solubility, good oral bioavailability (FIG. 38), good predicted potential for brain delivery, and the ability to achieve brain concentrations exceeding the EC₅₀ value by ≧10× (FIG. 42). From these, 10 were selected for multiple-dose pharmacokinetic studies focusing on “steady-state” concentrations (Cₛₛ) in brain homogenate, AUC in brain homogenate, and the ability to maintain Cₛₛ values ≧10× the EC₅₀ value over time. Several promising compounds were identified, including IND24, IND81, and IND22.

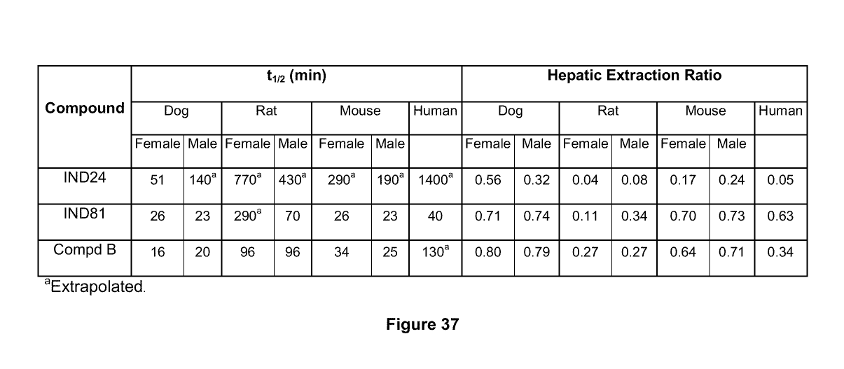

In general, all 10 compounds had low aqueous solubility at pH 2 or 4, but had good solubility in FaSSIF and in cell media, which was used as an indirect measure of solubility in the presence of proteins. Permeability studies were then performed for 10 selected compounds to get an initial assessment of any potential issues that would suggest that some might be substrates for P-gp. The efflux ratios suggested that none were substrates of P-gp. Metabolic stability was performed on the same 10 selected compounds to evaluate the t_{1/2} in mouse, rat and human microsomes, along with corresponding hepatic extraction ratios. As can be seen in FIG. 37, there was a wide range of stabilities in human, mice and rat microsomes among compounds. While dosing of IND24 and IND81 were well tolerated for over 200 days, we saw deaths in mice treated with IND22 as early as 59 days. Metabolic results suggested that IND24 should have excellent stability in humans and mice. While hydroxylated metabolites of IND24 could be identified in human liver microsomes, the role of any specific P450 isozyme could not be ascertained. Similarly, several hydroxylated metabolites could be identified for IND81 with CYP1A2, CYP2D6, CYP3A4, and CYP2C19 to a smaller extent implicated in its metabolism. Binding of IND24 and IND81 was evaluated in plasma and 20% brain homogenate at 1 and 10 μM. While the free fraction in plasma differed somewhat among mouse, rat, dog, and human, they were all in the same general range of 57%. The nonbound free fraction in brain ranged from 7-8% for IND24 and was 9% for IND81.

Pharmacokinetic studies were performed for IND24, IND81, IND22 and Compd B following IV (1 mg/kg) and oral (10 mg/kg) doses. The results show that the bioavailability of the three AMT and Compd B ranged from 25-46% in mice. The t_{1/2} was longer for the AMT compounds, especially IND24, following IV dosing (FIG. 38). AMT were studied in single dose oral pharmacokinetic studies at 40 or 10 mg/kg. Initially, 27 were dosed at 40 mg/kg in order to ensure that measurable concentrations would be above the lower level of quantitation by LC/MS/MS. Ten (including three that were repeated) were studied at 10 mg/kg. AUC_{0->last} ranged from 0.02-500 μM*h and <0.01-40 μM*h after the 40 and 10 mg/kg doses, respectively (FIG. 41).

IND24, IND81, and IND22 showed the highest AUC values in brain. In addition, the ratio of brain concentrations to EC₅₀ values obtained in neuronal cells after single oral doses of 40 or 10 mg/kg for all 34 analogs, ranged from 0.008-100 and <0.01-8, respectively (FIG. 42). Because IND24 and IND81 were among the best after the 40 and 10 mg/kg doses, and have good overall drug-like properties, including toleration, they were selected to advance to in-vivo animal studies to evaluate their potential to extend survival in the prion-infected models. IND85, a 2-AMT analog recently identified, actually had a better Cₘₐₓ:EC₅₀ ratio at either dose.

Experiments were performed to evaluate pharmacokinetics, including brain delivery, for the two optimized leads over a wide range of doses that could be used for 300 days, or longer. This would require the use of a liquid formulation and diet in order to minimize animal handling during a 300+ day study. To achieve good oral bioavailability and good target drug concentrations in the brain, we showed that it was necessary to add PEG400 to the oral formulation of the drug, which was added to the liquid diet. This enhanced drug dissolution (pH dependent solubility), absorption and oral bioavailability.

The most important pharmacokinetic studies were those involving three-day dosing to yield C concentrations in brain and plasma. Cₛₛ values well below the EC₅₀ will likely lead to sub-therapeutic drug concentrations in prion-infected mouse models, while concentrations >10×EC₅₀ would support a proof-of-concept experiment in the planned prion-infected mouse experiments. For IND24, IND81, and IND22 brain concentrations were above 10×EC₅₀ at doses between 50-125 mg/kg/day, where the dose of PEG400 was 0.125% (v/v) (FIG. 43). Linearity in brain and plasma concentrations was better for IND81. Interestingly, concentrations of Compd B did not increase linearly in brain or plasma suggesting that the metabolism of this drug is saturable.

KK. Compounds

### (4-Oxazol-5-yl-phenyl)-hydrazine

4-Oxazol-5-yl-phenylamine (100 mg, 0.62 mmol) in HC137% (3 mL) was cooled and stirred in an ice/salt bath (−20° C.). A pre-chilled solution of NaNO₂ (43 mg, 0.62 mmol) in H₂O (1 mL) was added dropwise in a period of 10 min. After the addition was completed a pre-cooled (−20° C.) solution of 5 nCl₂xH₂O (280 mg, 1.24 mmol, 2 eq) in HC137% (2 mL) was added dropwise to the solution. The reaction was stirred at −20° C. for 30 min. The solid was filtered and dissolved in water and basified with 5N NaOH and extracted with EtOAc (3×30 mL). The organic phase was dried (MgSO₄) and evaporated to afford 90 mg (90%) of the crude product that was used in the next step without further purification. ESI-MS m/z: 176 (M+H)⁺. ¹H-NMR (400 MHz, DMSO-d₆) δ: 4.10 (2H, br s), 6.82 (2H, d), 7.05 (1H, s), 7.30 (1H, s), 7.43 (2H, d), 8.25 (1H, s).

### N-(4-Oxazol-5-yl-phenyl)-N′-pyridin-4-ylmethylene-hydrazine (Compd B)

Pyridine-4-carbaldehyde (55 mg, 0.51 mmol) was added to an ethanol (3 mL) solution of (4-oxazol-5-yl-phenyl)-hydrazine (90 mg, 0.51 mmol). The reaction mixture was heated at 60° C. for 3 h. The solvent was evaporated and the solid was dissolved in ethyl acetate, cooled, and the precipitate filtered and washed with Et₂O to give 63 mg (76%) of the desired product as a yellow powder. ESI-MS m/z: 265 (M+H)⁺. ¹H-NMR (400 MHz, CDCl₃) δ: 7.20 (2H, d), 7.24 (1H, s), 7.52 (2H), 7.60 (2H, d), 7.63 (1H, s), 7.87 (1H, s), 8.06 (1H, br s), 8.60 (2H, d).

LL. Anti-Prion Drugs in Dividing and Nondividing Cells

The results indicate that fewer compounds are capable of clearing prions in nondividing cells in comparison to dividing cells. The previous study on the effect of cell division on prion accumulation provides a possible explanation for this effect.⁵⁰ In prion-infected cells, three competing processes influence prion accumulation: PrP^{C} to PrP^{Sc} conversion, PrP^{Sc} degradation and cell division. It had previously shown that for infected N2a cells the process of cell division plays an important role in establishing the steady state level of intracellular PrP^{Sc}. As an infected cell divides, the accumulated PrP^{Sc} is divided amongst the resulting daughter cells. Thus, cell division contributes to the apparent rate of prion clearance in a continuously dividing cell line. A compound that inhibits prion formation without affecting its clearance will appear more efficacious in dividing cell lines, as the steady state concentration of PrP^{Sc} is decreased during the process of cellular propagation. Conversely, in non-dividing cells, PrP^{Sc} catabolism is the sole route of prion clearance. In these cells, prion clearance due to the inhibition of prion formation will occur at a slower rate, reflecting the natural degradation rate of PrP^{Sc}.

The differential ability of anti-prion drugs to clear prions in dividing and nondividing cells had previously been demonstrated for quinacrine.³⁸ Whereas quinacrine rapidly clears dividing cells of PrP^{Sc}, it is relatively ineffective in nondividing prion-infected cells. The results suggested that the reduced rate of clearance in nondividing cells provide an opportunity for the formation of drug-resistant strains upon continuous exposure to quinacrine.

TABLE 29
2-AMT analogs synthesized and tested for potency with calculated parameters.

| Compound | Structure | EC₅₀ (μM)^{a} | MW | tPSA^{b} | xlogP | HBA/HBD^{c} |
|---|---|---|---|---|---|---|
| IND2 | 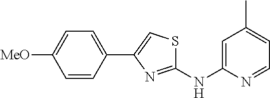 [SMILES: COc1ccc(-c2csc(Nc3cc(C)ccn3)n2)cc1] | 1.66 | 297.4 | 47.0 | 4.41 | 4/1 |
| IND7 | 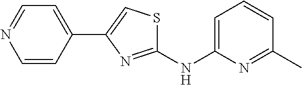 [SMILES: Cc1cccc(Nc2nc(-c3ccncc3)cs2)n1] | 1.00 | 268.3 | 50.7 | 3.05 | 4/1 |
| IND22 | 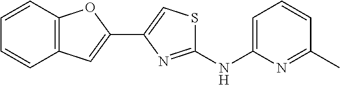 [SMILES: Cc1cccc(Nc2nc(-c3cc4ccccc4o3)cs2)n1] | 1.46 | 307.4 | 51.3 | 5.17 | 4/1 |
| IND24 | 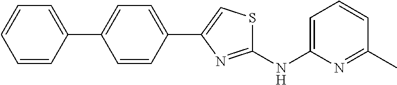 [SMILES: Cc1cccc(Nc2nc(-c3ccc(-c4ccccc4)cc3)cs2)n1] | 1.27 | 343.4 | 37.8 | 6.27 | 3/1 |
| IND26 | 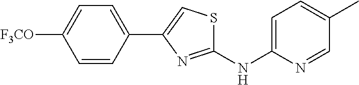 [SMILES: Cc1ccc(Nc2nc(-c3ccc(OC(F)(F)F)cc3)cs2)nc1] | >10.0 | 351.4 | 47.0 | 5.81 | 4/1 |
| IND29 | 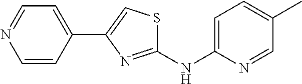 [SMILES: Cc1ccc(Nc2nc(-c3ccncc3)cs2)nc1] | 1.57 | 268.3 | 50.7 | 3.13 | 4/1 |
| IND33 | 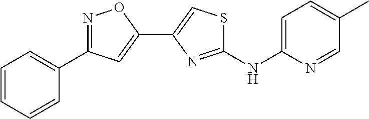 [SMILES: Cc1ccc(Nc2nc(-c3cc(-c4ccccc4)no3)cs2)nc1] | 0.94 | 334.4 | 64.1 | 4.29 | 5/1 |
| IND36 | 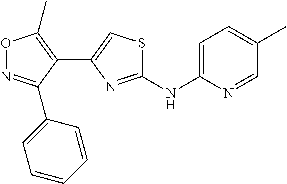 [SMILES: Cc1ccc(Nc2nc(-c3c(-c4ccccc4)noc3C)cs2)nc1] | >32.0 | 348.4 | 64.1 | 4.57 | 5/1 |
| IND38 | 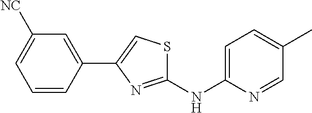 [SMILES: Cc1ccc(Nc2nc(-c3cccc(C#N)c3)cs2)nc1] | 2.29 | 292.4 | 61.6 | 4.16 | 4/1 |
| IND42 | 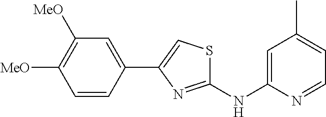 [SMILES: COc1ccc(-c2csc(Nc3cc(C)ccn3)n2)cc1OC] | 1.00 | 327.4 | 56.3 | 4.37 | 5/1 |
| IND43 | 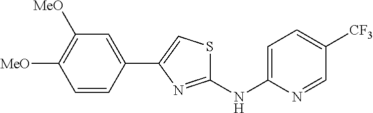 [SMILES: COc1ccc(-c2csc(Nc3ccc(C(F)(F)F)cn3)n2)cc1OC] | 2.53 | 381.4 | 56.3 | 5.08 | 5/1 |
| IND44 | 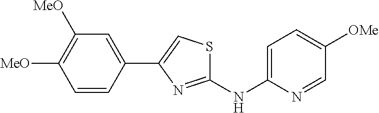 [SMILES: COc1ccc(Nc2nc(-c3ccc(OC)c(OC)c3)cs2)nc1] | 0.99 | 343.4 | 65.5 | 4.05 | 6/1 |
| IND46 | 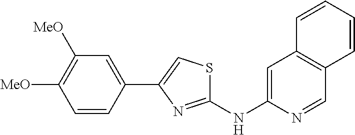 [SMILES: COc1ccc(-c2csc(Nc3cc4ccccc4cn3)n2)cc1OC] | 0.109 | 363.4 | 56.3 | 5.38 | 5/1 |
| IND47 | 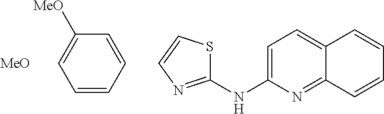 | 0.426 | 363.4 | 56.3 | 5.51 | 5/1 |
| IND48 | 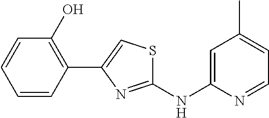 [SMILES: Cc1ccnc(Nc2nc(-c3ccccc3O)cs2)c1] | >10.0 | 283.4 | 58.0 | 4.07 | 4/2 |
| IND49 | 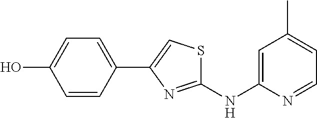 [SMILES: Cc1ccnc(Nc2nc(-c3ccc(O)cc3)cs2)c1] | >10.0 | 283.4 | 58.0 | 4.07 | 4/2 |
| IND52 | 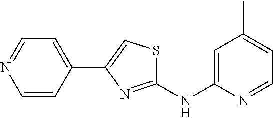 [SMILES: Cc1ccnc(Nc2nc(-c3ccncc3)cs2)c1] | 4.95 | 268.3 | 50.7 | 3.13 | 4/1 |
| IND57 | 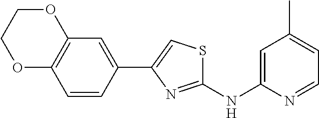 [SMILES: Cc1ccnc(Nc2nc(-c3ccc4c(c3)OCCO4)cs2)c1] | >10 | 325.4 | 56.3 | 3.95 | 5/1 |
| IND64 | 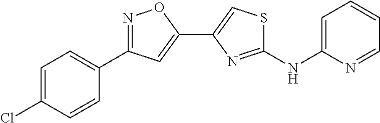 [SMILES: Clc1ccc(-c2cc(-c3csc(Nc4ccccn4)n3)on2)cc1] | 0.85 | 368.8 | 64.1 | 4.91 | 5/1 |
| IND74 | 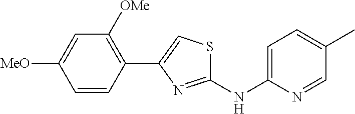 [SMILES: COc1ccc(-c2csc(Nc3ccc(C)cn3)n2)c(OC)c1] | 3.13 | 327.4 | 56.3 | 4.37 | 5/1 |
| IND76 | 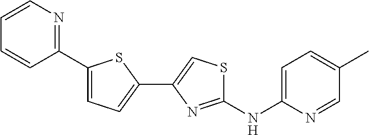 [SMILES: Cc1ccc(Nc2nc(-c3ccc(-c4ccccn4)s3)cs2)nc1] | 1.23 | 350.5 | 50.7 | 4.83 | 4/1 |
| IND78 | 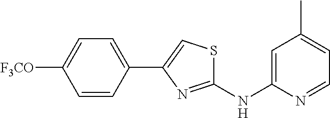 [SMILES: Cc1ccnc(Nc2nc(-c3ccc(OC(F)(F)F)cc3)cs2)c1] | >32.0 | 351.4 | 47.0 | 5.81 | 4/1 |
| IND81 | 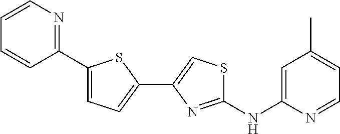 [SMILES: Cc1ccnc(Nc2nc(-c3ccc(-c4ccccn4)s3)cs2)c1] | 1.95 | 350.5 | 50.7 | 4.83 | 4/1 |
| IND82 | 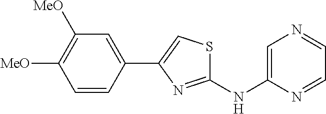 [SMILES: COc1ccc(-c2csc(Nc3cnccn3)n2)cc1OC] | 15.6 | 314.4 | 69.2 | 2.78 | 6/1 |
| IND85 | 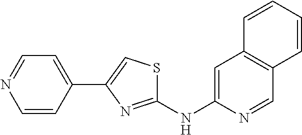 [SMILES: c1ccc2cc(Nc3nc(-c4ccncc4)cs3)ncc2c1] | 0.307 | 304.4 | 50.7 | 4.14 | 4/1 |
| IND86 | 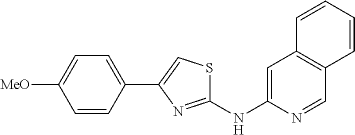 [SMILES: COc1ccc(-c2csc(Nc3cc4ccccc4cn3)n2)cc1] | 0.342 | 333.4 | 47.0 | 5.41 | 4/1 |
| IND91 | 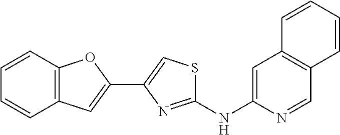 [SMILES: c1ccc2cc(Nc3nc(-c4cc5ccccc5o4)cs3)ncc2c1] | 0.923 | 343.4 | 51.3 | 6.25 | 4/1 |
| Compd B | 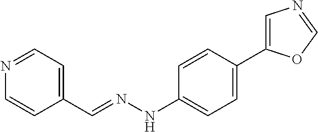 [SMILES: C(=N\Nc1ccc(-c2cnco2)cc1)/c1ccncc1] | 0.246 | 264.3 | 63.6 | 2.62 | 5/1 |

^{a}EC₅₀ values usually based on n = 3 or more.
^{b}tPSA = total polar surface area relating to N and O atoms (TPSA_NO) as calculated in Vortex v2011, Dotmatics Limited.
^{c}HBA = H-bond acceptor; HBD = H-bond donor.

TABLE 30
Metabolic stability of 9 selected 2-AMT compounds and Compd B.

| | t_{1/2} in min | | | | | |
|---|---|---|---|---|---|---|
| | (% remaining after | | | | | |
| Com- | a 60-min incubation) | | | Hepatic Extraction Ratio | | |
| pound | Mouse | Rat | Human | Mouse | Rat | Human |
| IND22 | 49 (40) | >60 (64) | >60 (99) | 0.7 | ND^{a} | ND |
| IND24 | >60 (89) | >60 (90) | >60 (105) | ND | ND | ND |
| IND33 | >60 (111) | >60 (73) | 54 (42) | ND | ND | 0.7 |
| IND44 | 29 (24) | 45 (39) | 58 (48) | 0.8 | 0.6 | 0.7 |
| IND46 | >60 (58) | >60 (61) | >60 (83) | ND | ND | ND |
| IND47 | >60 (56) | >60 (50) | >60 (113) | ND | ND | ND |
| IND52 | 6 (0) | 16 (8) | 36 (30) | 0.95 | 0.8 | 0.8 |
| IND81 | 19 (10) | 58 (48) | 52 (48) | 0.8 | 0.5 | 0.7 |
| IND85 | 15 (6) | 18 (32) | 53 (66) | 0.9 | 0.8 | 0.7 |
| Compd B | 30 (29) | >60 (77) | >60 (68) | 0.8 | ND | ND |

^{a}ND = Could not be accurately determined.

TABLE 31
Fraction unbound of IND24, IND81, and Compd B in mouse
brain homogenate, mouse and human plasma, and cell-
culture media at 1 μM. Mean percentage ± SD
based on n = 3, except for warfarin where n = 2.

| Compound | Sample | Unbound (%) |
|---|---|---|
| IND24 | Mouse Plasma | 6.63 ± 1.55 |
| | Mouse Brain Homogenate | 8.20 ± 0.35 |
| | Human Plasma | 4.91 ± 1.03 |
| | Cell-culture Media | 25.7 ± 4.93 |
| IND81 | Mouse Plasma | 6.48 ± 1.10 |
| | Mouse Brain Homogenate | 9.43 ± 1.49 |
| | Human Plasma | 5.78 ± 3.21 |
| | Cell-culture Media | 23.2 ± 2.68 |
| Compd B | Mouse Plasma | 9.06 ± 2.31 |
| | Mouse Brain Homogenate | 11.4 ± 2.89 |
| | Human Plasma | 2.10 ± 1.11 |
| | Cell-culture Media | 36.5 ± 3.30 |
| Warfarin | Mouse Plasma | 10.8 ± 0.0044 |
| | Human Plasma | 0.68 ± 0.0023 |

TABLE 32
Source and number of compounds tested in each HTS assay.

| Assay | Chembridge^{a} | SPECS^{b} | Total | FDA-Drugs |
|---|---|---|---|---|
| PrP^{Sc} | 22,703 | 30,127 | 52,830 | 1,420 |
| (dividing cells) | | | | |
| PrP^{Sc} | 19,327 | 30,103 | 49,430 | 1,420 |
| (stationary-phase cells) | | | | |

^{a}Chembridge library = 23,861 compounds
^{b}SPECS library = 30,256 compounds

TABLE 33
The specific structures for the 14 leads identified in dividing ScN2a-cl3 cells in the PrP^{Sc} assay, with corresponding EC₅₀
results from ELISA and Western immunoblots, LC₅₀ values from calcein assays, and Qikprop predictions of
physicochemical properties. (QPCaco-2 values expressed as 10⁻⁶ cm/s and PSA in angstroms.)

| | | | | | | | Calculated Physicochemical | | | | |
|---|---|---|---|---|---|---|---|---|---|---|---|
| Lead | | | Potency (nM) | | | | Parameters^{a} | | | | |
| (Murcko | Com- | | | | Wes- | | | | HBD/ | QP | QPlog |
| Fragment) | pound | Structure | MW | ELISA | tern | Calcein | ClogP | PSA | HBA | Caco-2 | BBB |
| Aminothiazole 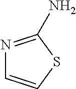 | IND- 30410 | 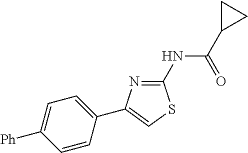 [SMILES: O=C(Nc1nc(-c2ccc(-c3ccccc3)cc2)cs1)C1CC1] | 320.4 | 317 | 194 | >10,000 | 4.45 | 47.9 | 1/4 | 2730.9 | −0.122 |
| | IND- 30883 | 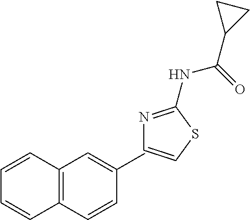 | 294.4 | 913 | 570 | >10,000 | 3.79 | 47.9 | 1/4 | 2728.5 | −0.036 |
| Thiazole 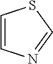 | IND- 24350 | 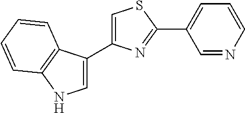 [SMILES: c1cncc(-c2nc(-c3c[nH]c4ccccc34)cs2)c1] | 277.3 | 321 | 828 | >10,000 | 3.75 | 37.5 | 1/3 | 2469.0 | 0.081 |
| | IND- 24352 | 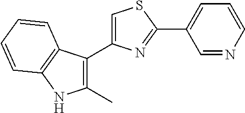 [SMILES: Cc1[nH]c2ccccc2c1-c1csc(-c2cccnc2)n1] | 291.4 | 2267 | 1698 | >10,000 | 4.06 | 35.8 | 1/3 | 2996.6 | 0.163 |
| Benzoxazole 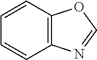 | IND- 114202 | 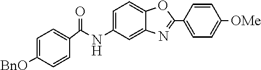 [SMILES: COc1ccc(-c2nc3cc(NC(=O)c4ccc(OCc5ccccc5)cc4)ccc3o2)cc1] | 450.5 | 57 | 2565 | >10,000 | 6.13 | 71.7 | 1/6 | 2564.3 | −0.538 |
| | IND- 114138 | 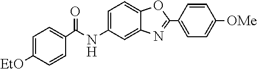 [SMILES: CCOc1ccc(C(=O)Nc2ccc3oc(-c4ccc(OC)cc4)nc3c2)cc1] | 388.4 | 65 | 134 | >10,000 | 4.69 | 72.4 | 1/6 | 2447.5 | −0.456 |
| Pyrazole 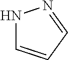 | IND- 25807 | 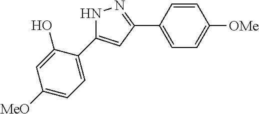 [SMILES: COc1ccc(-c2cc(-c3ccc(OC)cc3O)[nH]n2)cc1] | 296.3 | 167 | 95 | >10,000 | 3.47 | 63.8 | 2/3 | 1586.2 | −0.415 |
| | IND- 28801 | 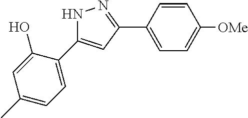 [SMILES: COc1ccc(-c2cc(-c3ccc(C)cc3O)[nH]n2)cc1] | 280.3 | 245 | 117 | >10,000 | 3.71 | 55.5 | 2/3 | 1586.8 | −0.357 |
| Thienopyridine 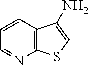 | IND- 52025 | 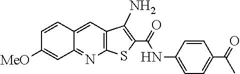 [SMILES: COc1ccc2cc3c(N)c(C(=O)Nc4ccc(C(C)=O)cc4)sc3nc2c1] | 391.4 | 57 | 61 | >10,000 | 2.80 | 109.9 | 2/6 | 72.0 | −0.950 |
| | IND- 52851 | 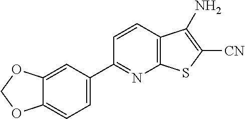 [SMILES: N#Cc1sc2nc(-c3ccc4c(c3)OCO4)ccc2c1N] | 295.3 | 962 | 518 | >10,000 | 1.48 | 81.6 | 1.5/5 | 131.5 | −0.276 |
| Piperazine 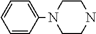 [SMILES: c1ccc(N2CCNCC2)cc1] | IND- 18629 | 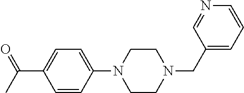 [SMILES: CC(=O)c1ccc(N2CCN(Cc3cccnc3)CC2)cc1] | 295.4 | 294.3 | 886 | >10,000 | 2.09 | 49.6 | 0/7 | 425.6 | 0.014 |
| | IND- 19136 | 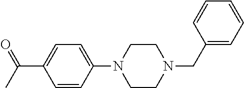 [SMILES: CC(=O)c1ccc(N2CCN(Cc3ccccc3)CC2)cc1] | 294.4 | 8681 | 615 | >10,000 | 3.16 | 36.8 | 0/5 | 784.9 | 0.269 |
| Imidazo- pyridine 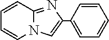 [SMILES: c1ccc(-c2cn3ccccc3n2)cc1] | IND- 9419 | 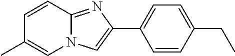 [SMILES: CCc1ccc(-c2cn3cc(C)ccc3n2)cc1] | 236.3 | 709 | 285 | >10,000 | 4.53 | 14.7 | 0/2 | 6882.3 | 0.346 |
| | IND- 9417 | 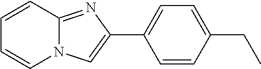 [SMILES: CCc1ccc(-c2cn3ccccc3n2)cc1] | 222.3 | 9158 | 4715 | >10,000 | 4.26 | 14.7 | 0/2 | 6882.6 | 0.351 |
| Imidazo- thiazole 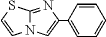 [SMILES: c1ccc(-c2cn3ccsc3n2)cc1] | IND- 9416 | 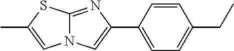 [SMILES: CCc1ccc(-c2cn3cc(C)sc3n2)cc1] | 242.3 | 1776 | 517 | >10,000 | 4.43 | 14.7 | 0/2 | 6469.8 | 0.438 |
| | IND- 9413 | 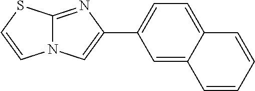 [SMILES: c1ccc2cc(-c3cn4ccsc4n3)ccc2c1] | 250.2 | 6015 | 2490 | >10,000 | 4.28 | 14.7 | 0/2 | 6473.8 | 0.523 |
| Guanidine 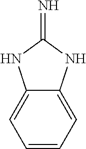 | IND- 26326 | 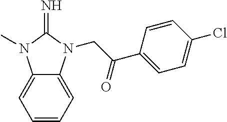 [SMILES: Cn1c(=N)n(CC(=O)c2ccc(Cl)cc2)c2ccccc21] | 299.8 | 5900 | 4967 | >10,000 | 4.07 | 53.8 | 1/3 | 1928.0 | −0.21 |
| | IND- 44723 | 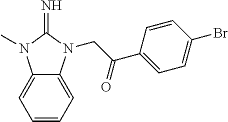 [SMILES: Cn1c(=N)n(CC(=O)c2ccc(Br)cc2)c2ccccc21] | 344.2 | 1983 | 1453 | >10,000 | 4.14 | 53.8 | 1/3 | 1928.1 | −0.20 |
| Quinoline 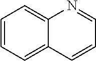 | IND- 45193 | 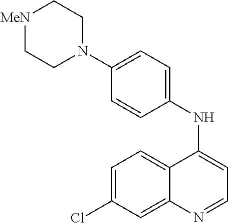 [SMILES: CN1CCN(c2ccc(Nc3ccnc4cc(Cl)ccc34)cc2)CC1] | 352.9 | 1220 | 2073 | >10,000 | 4.36 | 30.2 | 1/5 | 1187.2 | 0.677 |
| | IND- 44355 | 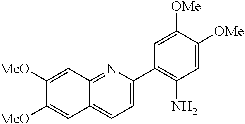 [SMILES: COc1cc(N)c(-c2ccc3cc(OC)c(OC)cc3n2)cc1OC] | 340.4 | 2143 | 1833 | >10,000 | 3.72 | 64.1 | 1.5/5 | 2957.7 | −0.339 |
| Quinazoline 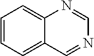 | IND- 34921 | 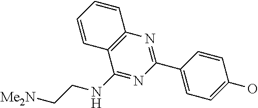 [SMILES: CN(C)CCNc1nc(-c2ccc(O)cc2)nc2ccccc12] | 326.8 | 3693 | 2009 | >10,000 | 4.00 | 33.4 | 1/5 | 1436.2 | 0.595 |
| | IND- 35916 | 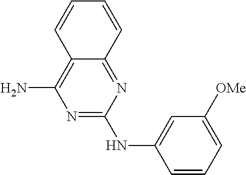 [SMILES: COc1cccc(Nc2nc(N)c3ccccc3n2)c1] | 266.3 | 4023 | 5987 | >10,000 | 2.60 | 63.9 | 3/4 | 1447.2 | −0.499 |
| Benzamide 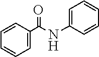 [SMILES: O=C(Nc1ccccc1)c1ccccc1] | IND- 33145 | 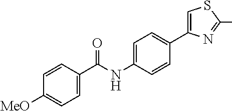 [SMILES: COc1ccc(C(=O)Nc2ccc(-c3csc(C)n3)cc2)cc1] | 324.4 | 412 | 161 | >10,000 | 4.09 | 54.9 | 1/5 | 2923.7 | −0.063 |
| | IND- 31751 | 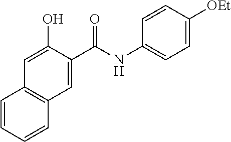 [SMILES: CCOc1ccc(NC(=O)c2cc3ccccc3cc2O)cc1] | 307.3 | 156 | 42 | >10,000 | 4.38 | 63.0 | 1/3 | 1544.2 | −0.607 |
| Benzyl ether 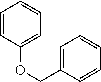 [SMILES: c1ccc(COc2ccccc2)cc1] | IND- 46380 | 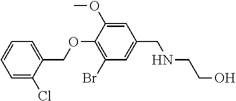 [SMILES: COc1cc(CNCCO)cc(Br)c1OCc1ccccc1Cl] | 400.7 | 5080 | 2148 | >10,000 | 3.95 | 49.7 | 2/5 | 557.1 | −0.004 |
| | IND- 34452 | 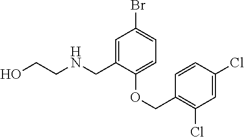 [SMILES: OCCNCc1cc(Br)ccc1OCc1ccc(Cl)cc1Cl] | 405.1 | 2850 | 1993 | 27% | 4.24 | 41.7 | 2/4 | 581.2 | 0.3 |
| Stilbene 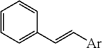 | IND- 47979 | 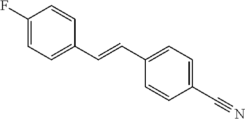 [SMILES: N#Cc1ccc(/C=C/c2ccc(F)cc2)cc1] | 223.3 | 1700 | 528 | >10,000 | 3.77 | 25.8 | 0/2 | 2055.6 | −0.229 |
| | IND- 47971 | 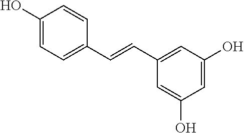 [SMILES: Oc1ccc(/C=C/c2cc(O)cc(O)c2)cc1] | 228.2 | 2260 | 584 | >10,000 | 2.01 | 67.6 | 3/2 | 277.0 | −0.129 |

^{a}Physicochemical parameters calculated using Qikprop (Schrödinger, New York, NY) {Barreiro, 2007 #9027; Dzierba, 2007 #9028}.

TABLE 34
One hundred-thirty and six hits identified in dividing and stationary-
phase cells, respectively, 15 confirmed hits in SPC hits in dividing
cells (0 in stationary-phase cells), and 15 tested in EC⁵⁰ for
potency from 1,420 FDA-approved drugs tested in the ScN2a-c13
assays in dividing and stationary-phase cells.

| | Potency (μM)^{a} | | |
|---|---|---|---|
| | | Cell | Calcein/ |
| FDA-Approved Drug | ELISA | Viability | ELISA Ratio |
| Dextran | 0.401 (mg/L) | >10 (mg/L) | >24.9 |
| Congo Red | 0.452 | >10 | >22 |
| Carvedilol | 2.282 | 6.096 | 2.67 |
| Tetrandine | 2.145 | 5.009 | 2.33 |
| Ethoxazine | 4.759 | >10 | >2.1 |
| Dihydroergotamine | 6.435 | >10 | >1.55 |
| Acepromazine | 6.928 | >10 | >1.44 |
| Amlodipine | 8.216 | >10 | >1.22 |
| Fendiline | 4.346 | 4.330 | 0.99 |
| Tamoxifen | 4.389 | 4.885 | 1.11 |
| Desloratidine | 6.658 | 9.561 | 1.44 |
| Apomorphine | >10 | >10 | nd^{b} |
| Amiodarone | >10 | >10 | nd |
| Hexadimethrine | >10 | >10 | nd |
| Enoxaparin | >10 (mg/L) | >10 (mg/L) | nd |

^{a}Units are all (μM, except those indicated by mg/L, since drugs are a mixture of molecular weights.
^{b}Not determinable.

P04156 (Homo sapien Prion Protein, PrP)

| | |
|---|---|
| 1 | manlgcwmlv lfvatwsdlg lckkrpkpgg wntggsrypg |
| | qgspggnryp pqggggwgqp |
| 61 | hgggwgqphg ggwgqphggg wgqphgggwg qgggthsqwn |
| | kpskpktnmk hmagaaaaga |
| 121 | vvgglggyml gsamsrpiih fgsdyedryy renmhrypnq |
| | vyyrpmdeys nqnnfvhdcv |
| 181 | nitikqhtvt tttkgenfte tdvkmmervv eqmcitqyer |
| | esqayyqrgs smvlfssppv |
| 241 | illisflifl ivg |

P04925 (Mouse Prion Protein)

| | |
|---|---|
| 1 | manlgywlla lfvtmwtdvg lckkrpkpgg wntggsrypg |
| | qgspggnryp pqggtwgqph |
| 61 | gggwgqphgg swgqphggsw gqphgggwgq gggthnqwnk |
| | pskpktnlkh vagaaaagav |
| 121 | vgglggymlg samsrpmihf gndwedryyr enmyrypnqv |
| | yyrpvdqysn qnnfvhdcvn |
| 181 | itikqhtvtt ttkgenftet dvkmmervve qmcvtqyqke |
| | sqayydgrrs sstvlfsspp |
| 241 | villisflif livg |

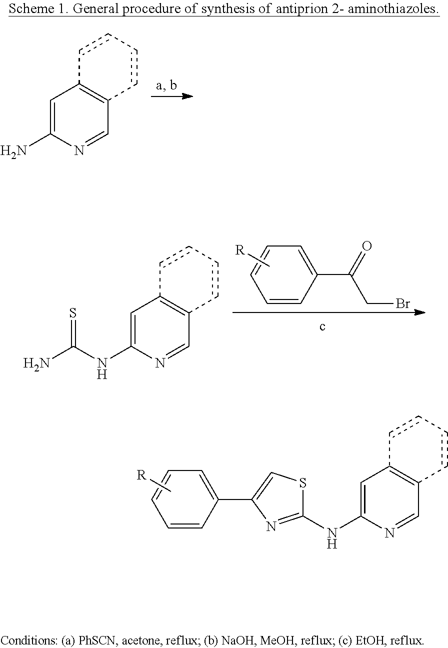

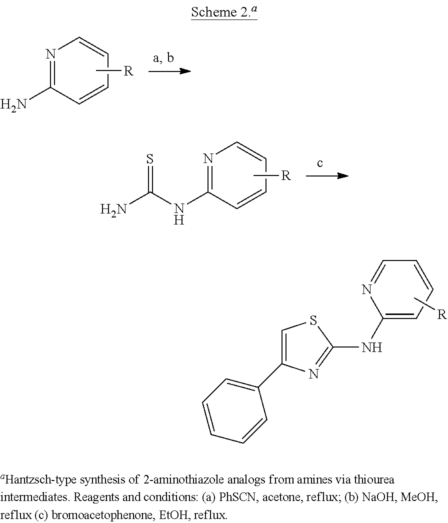

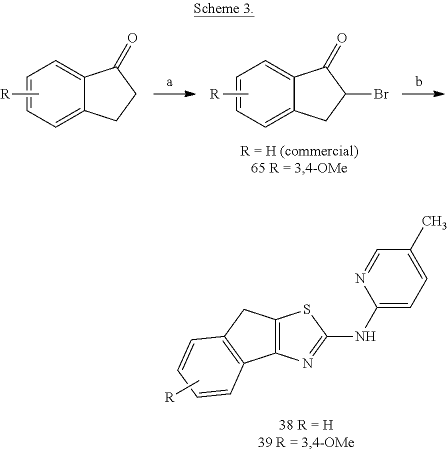

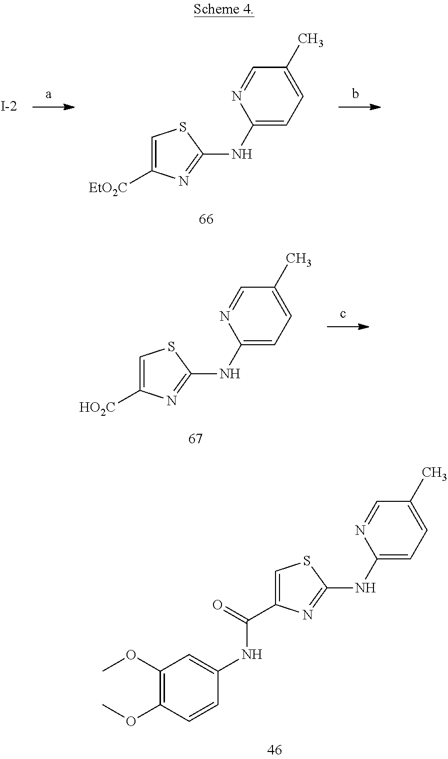

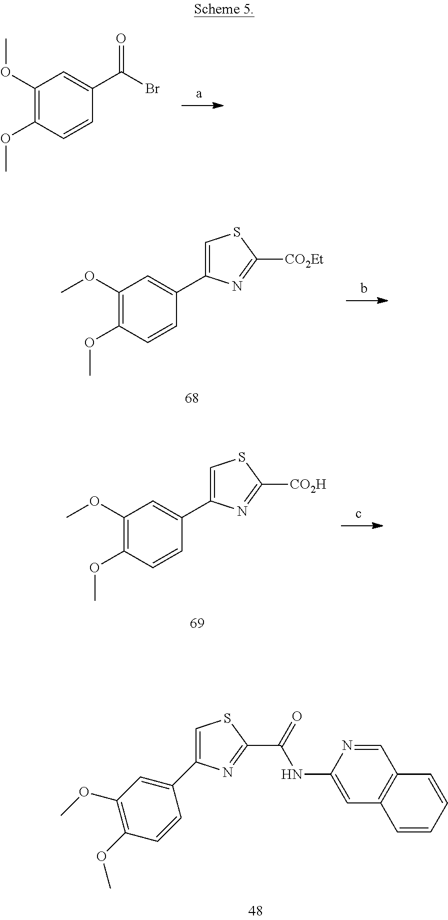 [SMILES: COc1ccc(C(=O)Br)cc1OC ; CCOC(=O)c1nc(-c2ccc(OC)c(OC)c2)cs1 ; COc1ccc(-c2csc(C(=O)O)n2)cc1OC ; COc1ccc(-c2csc(C(=O)Nc3cc4ccccc4cn3)n2)cc1OC]

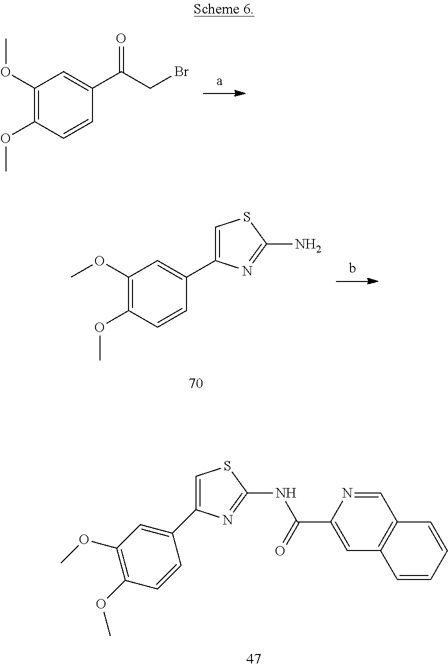 [SMILES: COc1ccc(C(=O)CBr)cc1OC ; COc1ccc(-c2csc(N)n2)cc1OC ; COc1ccc(-c2csc(NC(=O)c3cc4ccccc4cn3)n2)cc1OC]

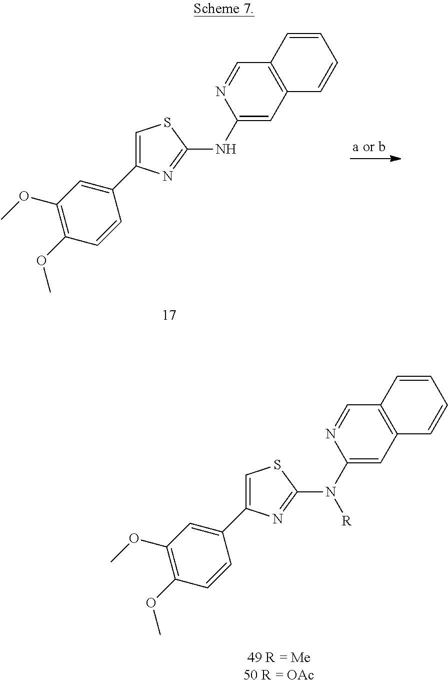

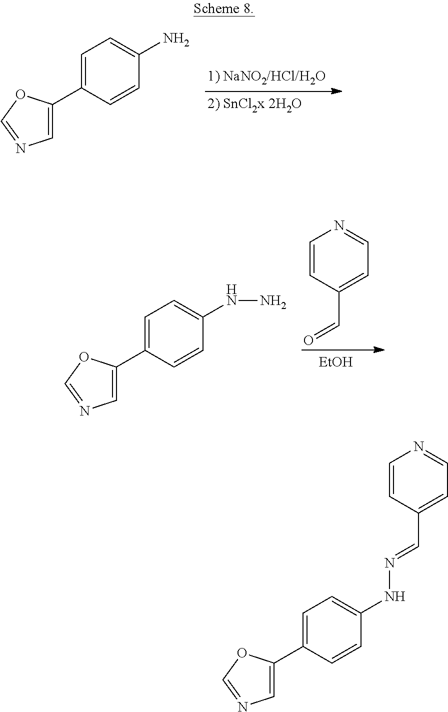 [SMILES: "EtOH" ; Nc1ccc(-c2cnco2)cc1 ; NNc1ccc(-c2cnco2)cc1 ; O=Cc1ccncc1 ; C(=N\Nc1ccc(-c2cnco2)cc1)/c1ccncc1]

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound having the formula: 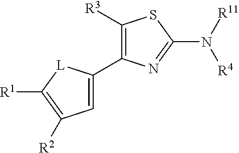
wherein,
L is —CR⁶═CH—, —S—, or —O—; R¹ is substituted or unsubstituted cycloalkyl, substituted or unsubstituted heterocycloalkyl, substituted or unsubstituted aryl, or substituted or unsubstituted heteroaryl;
R², R³ and R⁶ are independently hydrogen,
halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, —C(O)CH₃, substituted or unsubstituted (C₁-C₄)alkyl, substituted or unsubstituted (C₃-C₆)cycloalkyl, or aryl;
R⁷, R⁸, R⁹, and R¹⁰ are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ is substituted or unsubstituted heteroaryl or —C(O)R¹²;
R¹² is substituted or unsubstituted cycloalkyl;
v is independently 1 or 2;
m is independently an integer from 1 to 2;
n is independently an integer from 0 to 4;
X is independently —Cl, —Br, —I, or —F.

2. (canceled)

3. (canceled)

4. The compound of claim 1, wherein R¹¹ is substituted or unsubstituted heteroaryl.

5. The compound of claim 1 having the formula: 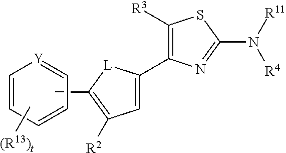
wherein,
R¹³ is independently hydrogen,
halogen, —CX3, —CN, —SO₂Cl, —SOᵣR¹⁷, —SOₚNR¹⁴R¹⁵, —NHNH₂, —ONR¹⁴R¹⁵, —NHC═(O)NHNH₂, —N HC═(O)NR¹⁴R¹⁵, —N(O)_{q}, —NR¹⁴R¹⁵, —C(O)R¹⁶, —C(O)—OR¹⁶, —C(O)NR¹⁴R¹⁵, —OR¹⁷, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
two adjacent R¹³ substituents may optionally be joined to form a substituted or unsubstituted cycloalkyl, substituted or unsubstituted heterocycloalkyl, substituted or unsubstituted aryl, or substituted or unsubstituted heteroaryl;
R¹⁴, R¹⁵, R¹⁶, and R¹⁷ are independently hydrogen,
halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂— substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
t is independently an integer from 0 to 5;
p is independently 1 or 2;
q is independently an integer from 1 to 2;
r is independently an integer from 0 to 4;
X is independently —Cl, —Br, —I, or F;
Y is independently —N═ or —N⁺(O—)—.

6. The compound of claim 1 having the formula: 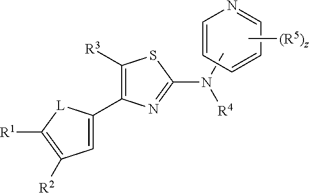
wherein,
R⁵ is independently hydrogen, halogen, —CX^{b}₃, —CN, —SO₂Cl, —SOₙ₁R^{10b}, —SOᵥ₁NR^{7b}R^{8b}, —NHNH₂, —ONR^{7b}R^{8b}, —NHC═(O)NHNH₂, —NHC═(O)NR^{7b}R^{8b}, —N(O)ₘ₁, —NR^{7b}R^{8b}, —C(O)—R^{9b}, —C(O)—OR^{9b}, —C(O)NR^{7b}R^{8b}, —OR^{10b} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R^{7b}, R^{8b}, R^{9b}, and R^{10b} are independently hydrogen,
halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z is independently an integer from 0 to 4;
v1 is independently 1 or 2;
m1 is independently an integer from 1 to 2;
n1 is independently an integer from 0 to 4;
X^{b} is independently —Cl, —Br, —I, or —F.

7.-11. (canceled)

12. The compound of claim 6, wherein R² is hydrogen.

13. The compound of claim 6, wherein R¹ is substituted or unsubstituted aryl or substituted or unsubstituted heteroaryl.

14.-25. (canceled)

26. The compound of claim 6, having the formula:

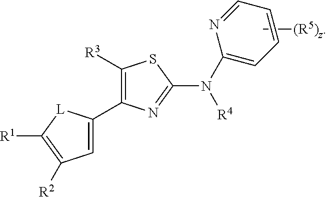

27. The compound of claim 26, wherein R⁵ is unsubstituted alkyl or substituted or unsubstituted heteroalkyl.

28.-33. (canceled)

34. The compound of claim 26, wherein z is 1.

35. The compound of claim 6, having the formula:

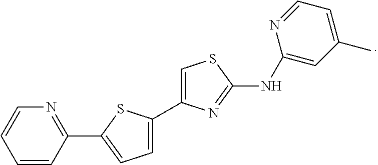 [SMILES: Cc1ccnc(Nc2nc(-c3ccc(-c4ccccn4)s3)cs2)c1]

36. The compound of claim 6, having the formula:

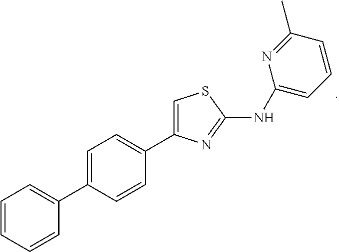 [SMILES: Cc1cccc(Nc2nc(-c3ccc(-c4ccccc4)cc3)cs2)n1]

37. A pharmaceutical composition comprising a pharmaceutically acceptable excipient and a compound of claim 1.

38. A method of treating a disease in a patient in need of such treatment, said method comprising administering a therapeutically effective amount of a compound of claim 1.

39. The method of claim 38, wherein the disease is a neurodegenerative disease.

40. The method of claim 39, wherein the disease is a prion disease.

41. (canceled)

42. The method of claim 39, wherein the disease is Alzheimer's disease, Amyotrophic lateral sclerosis, Huntington's disease, or Parkinson's disease.

43.-108. (canceled)
